(19) United States
(12) Patent Application Publication
(10) Pub. No.: US 2022/0098329 A1
(43) Pub. Date: Mar. 31, 2022

(54) HETERODIMERIC TETRAVALENCY AND SPECIFICITY ANTIBODY COMPOSITIONS AND USES THEREOF

(71) Applicant: Memorial Sloan Kettering Cancer Center, New York, NY (US)

(72) Inventors: Brian Santich, New York, NY (US); Nai-Kong V. Cheung, New York, NY (US); Morgan Huse, New York, NY (US)

(73) Assignee: Memorial Sloan Kettering Cancer Center, New York, NY (US)

(21) Appl. No.: 17/298,008

(22) PCT Filed: Nov. 29, 2019
(86) PCT No.: PCT/US2019/063854

§ 371 Date: May 27, 2021

Related U.S. Application Data
(60) Provisional application No. 62/774,111, filed on Nov. 30, 2018.
(60) Provisional application No. 62/794,523, filed on Jan. 18, 2019.

(51) Int. Cl. C07K 16/46; C07K 16/28; C07K 16/30; C07K 16/32; A61P 35/00; A61K 39/395

(52) U.S. Cl. CPC C07K 16/468; C07K 16/2809; A61K 39/39558; C07K 16/32; A61P 35/00; C07K 16/3084

(57) ABSTRACT

The present disclosure relates generally to immunoglobulin-related compositions (e.g., heterodimeric trivalent/tetravalent multispecific antibodies) that specifically bind to three or four distinct target antigens. The immunoglobulin-related compositions described herein are useful in methods for detecting and treating cancer in a subject in need thereof.

### CROSS-REFERENCE TO RELATED PATENT APPLICATIONS

This application is a U.S. National Stage Application under 35 U.S.C. § 371 of International Patent Application No. PCT/US2019/063854, filed on Nov. 29, 2019, which claims the benefit of and priority to US Provisional Appl. Nos. 62/774,111, filed Nov. 30, 2018, and 62/794,523, filed Jan. 18, 2019, the disclosure of each of which are incorporated by reference herein in its entirety.

### SEQUENCE LISTING

The instant application contains a Sequence Listing which has been submitted electronically in ASCII format and is hereby incorporated by reference in its entirety. Said ASCII copy, created on Dec. 23, 2019, is named 115872-0497 SL.txt and is 1,200,059 bytes in size.

### TECHNICAL FIELD

The present technology relates generally to the preparation of heterodimeric trivalent/tetravalent multispecific antibodies that specifically bind three or four distinct target antigens, and their uses. The heterodimeric trivalent/tetravalent multispecific antibodies described herein are useful in methods for detecting and treating cancer in a subject in need thereof.

### BACKGROUND

The following description of the background of the present technology is provided simply as an aid in understanding the present technology and is not admitted to describe or constitute prior art to the present technology.

Many antibody platforms exist, including heterodimeric IgG and BiTE. See Spiess et al., Mol Immunol 67:95-106 (2015); Shima et al., N Engl J Med 374:2044-2053 (2016); Topp et al., Lancet Oncol 16:57-66 (2015). However, no single antibody platform to date has shown a clear and significant functional advantage over others within the clinic.

In the case of multispecific antibodies that engage immune cells, such as BiTEs, the ideal structure that maximizes anti-tumor activity has not been defined, and likely varies based on the target antigens or the parental antibodies (Wu & Cheung, Pharmacology & Therapeutics 182:161-175 (2018). Important properties may include antigen size and proximity to the cell membrane as well as serum half-life. See Bluemel et al., Cancer Immunol Immunother 59:1197-1209 (2010); Suzuki et al., J Immunol 184:1968-1976 (2010); Yang et al., Cancer Res 64:6673-6678 (2004). Even less is understood about the spatial orientation imparted by the antibody on the cell-to-cell interface, the strength of each individual specificity interaction, or the number of interactions. Moreover, the size of the antibody format, the flexibility of each binding domain, and their relative orientations to one another may influence the capacity to properly or effectively engage multiple antigens at once. Given these different complexities, it is of paramount importance to understand if a given platform design is properly optimized for therapeutic function.

### Summary of the Present Technology

In one aspect, the present disclosure provides a heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein: (a) the first polypeptide chain comprises in the N-terminal to C-terminal direction: (i) a light chain variable domain of a first immunoglobulin (VL-1) that is capable of specifically binding to a first epitope; (ii) a light chain constant domain of the first immunoglobulin (CL-1); (iii) a flexible peptide linker comprising the amino acid sequence (GGGGS)₃ (SEQ ID NO: 2506); and (iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ (SEQ ID NO: 2507) to form a single-chain variable fragment; (b) the second polypeptide comprises in the N-terminal to C-terminal direction: (i) a heavy chain variable domain of the first immunoglobulin (VH-1) that is capable of specifically binding to the first epitope; (ii) a first CH1 domain of the first immunoglobulin (CH1-1); and (iii) a first heterodimerization domain of the first immunoglobulin, wherein the first heterodimerization domain is incapable of forming a stable homodimer with another first heterodimerization domain; (c) the third polypeptide comprises in the N-terminal to C-terminal direction: (i) a heavy chain variable domain of a third immunoglobulin (VH-3) that is capable of specifically binding to a third epitope; (ii) a second CH1 domain of the third immunoglobulin (CH1-3); and (iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin; (d) the fourth polypeptide comprises in the N-terminal to C-terminal direction: (i) a light chain variable domain of the third immunoglobulin (VL-3) that is capable of specifically binding to the third epitope; (ii) a light chain constant domain of the third immunoglobulin (CL-3); (iii) a flexible peptide linker comprising the amino acid sequence (GGGGS)₃ (SEQ ID NO: 2506); and (iv) a light chain variable domain of a fourth immunoglobulin (VL-4) that is linked to a complementary heavy chain variable domain of the fourth immunoglobulin (VH-4), or a heavy chain variable domain of a fourth immunoglobulin (VH-4) that is linked to a complementary light chain variable domain of the fourth immunoglobulin (VL-4), wherein VL-4 and VH-4 are capable of specifically binding to the second epitope, and are linked together via a flexible peptide linker comprising the amino acid sequence (GGGGS)₆ (SEQ ID NO: 2507) to form a single-chain variable fragment, and wherein each of VL-1 and VL-3 independently comprises the CDR1 sequence, the CDR2 sequence and the CDR3 sequence of a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345; and/or wherein each of VH-1 and VH-3 independently comprises the CDR1 sequence, the CDR2 sequence and the CDR3 sequence of a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349.

In one aspect, the present disclosure provides a heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein: (a) the first polypeptide chain comprises in the N-terminal to C-terminal direction: (i) a light chain variable domain of a first immunoglobulin (VL-1) that is capable of specifically binding to a first epitope; (ii) a light chain constant domain of the first immunoglobulin (CL-1); (iii) a flexible peptide linker comprising the amino acid sequence (GGGGS)₃ (SEQ ID NO: 2506); and (iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein the VL-2 and VH-2 are capable of specifically binding to a second epitope, and are linked together via a flexible peptide linker comprising the amino acid sequence (GGGGS)₆ (SEQ ID NO: 2507) to form a single-chain variable fragment; (b) the second polypeptide comprises in the N-terminal to C-terminal direction: (i) a heavy chain variable domain of the first immunoglobulin (VH-1) that is capable of specifically binding to the first epitope; (ii) a first CH1 domain of the first immunoglobulin (CH1-1); and (iii) a first heterodimerization domain of the first immunoglobulin, wherein the first heterodimerization domain is incapable of forming a stable homodimer with another first heterodimerization domain; (c) the third polypeptide comprises in the N-terminal to C-terminal direction: (i) a heavy chain variable domain of a third immunoglobulin (VH-3) that is capable of specifically binding to the first epitope; (ii) a second CH1 domain of the third immunoglobulin (CH1-3); and (iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin; (d) the fourth polypeptide comprises in the N-terminal to C-terminal direction: (i) a light chain variable domain of the third immunoglobulin (VL-3) that is capable of specifically binding to the first epitope; (ii) a light chain constant domain of the third immunoglobulin (CL-3); (iii) a flexible peptide linker comprising the amino acid sequence (GGGGS)₃ (SEQ ID NO: 2506); and (iv) a light chain variable domain of a fourth immunoglobulin (VL-4) that is linked to a complementary heavy chain variable domain of the fourth immunoglobulin (VH-4), or a heavy chain variable domain of a fourth immunoglobulin (VH-4) that is linked to a complementary light chain variable domain of the fourth immunoglobulin (VL-4), wherein the VL-4 and VH-4 are capable of specifically binding to a third epitope, and are linked together via a flexible peptide linker comprising the amino acid sequence (GGGGS)₆ (SEQ ID NO: 2507) to form a single-chain variable fragment, and wherein each of VL-2 and VL-4 independently comprises the CDR1 sequence, the CDR2 sequence and the CDR3 sequence of a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345; and/or wherein each of VH-2 and VH-4 independently comprises the CDR1 sequence, the CDR2 sequence and the CDR3 sequence of a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349.

In another aspect, the present disclosure provides a heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein: (a) the first polypeptide chain comprises in the N-terminal to C-terminal direction: (i) a light chain variable domain of a first immunoglobulin (VL-1) that is capable of specifically binding to a first epitope; (ii) a light chain constant domain of the first immunoglobulin (CL-1); (iii) a flexible peptide linker comprising the amino acid sequence (GGGGS)₃ (SEQ ID NO: 2506); and (iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ (SEQ ID NO: 2507) to form a single-chain variable fragment; (b) the second polypeptide comprises in the N-terminal to C-terminal direction: (i) a heavy chain variable domain of the first immunoglobulin (VH-1) that is capable of specifically binding to the first epitope; (ii) a first CH1 domain of the first immunoglobulin (CH1-1); and (iii) a first heterodimerization domain of the first immunoglobulin, wherein the first heterodimerization domain is incapable of forming a stable homodimer with another first heterodimerization domain; (c) the third polypeptide comprises in the N-terminal to C-terminal direction: (i) a heavy chain variable domain of a third immunoglobulin (VH-3) that is capable of specifically binding to a third epitope; (ii) a second CH1 domain of the third immunoglobulin (CH1-3); and (iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin; (d) the fourth polypeptide comprises in the N-terminal to C-terminal direction: (i) a light chain variable domain of the third immunoglobulin (VL-3) that is capable of specifically binding to the third epitope; (ii) a light chain constant domain of the third immunoglobulin (CL-3); (iii) a flexible peptide linker comprising the amino acid sequence (GGGGS)₃ (SEQ ID NO: 2506); and (iv) a light chain variable domain of a fourth immunoglobulin (VL-4) that is linked to a complementary heavy chain variable domain of the fourth immunoglobulin (VH-4), or a heavy chain variable domain of a fourth immunoglobulin (VH-4) that is linked to a complementary light chain variable domain of the fourth immunoglobulin (VL-4), wherein VL-4 and VH-4 are capable of specifically binding to the fourth epitope, and are linked together via a flexible peptide linker comprising the amino acid sequence (GGGGS)₆ (SEQ ID NO: 2507) to form a single-chain variable fragment; and wherein each of VL-1 and VL-3 independently comprises the CDR1 sequence, the CDR2 sequence and the CDR3 sequence of a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345; and/or wherein each of VH-1 and VH-3 independently comprises the CDR1 sequence, the CDR2 sequence and the CDR3 sequence of a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349; and/or wherein each of VL-2 and VL-4 independently comprises the CDR1 sequence, the CDR2 sequence and the CDR3 sequence of a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345; and/or wherein each of VH-2 and VH-4 independently comprises the CDR1 sequence, the CDR2 sequence and the CDR3 sequence of a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349.

In another aspect, the present disclosure provides a heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein: (a) the first polypeptide chain comprises in the N-terminal to C-terminal direction: (i) a light chain variable domain of a first immunoglobulin (VL-1) that is capable of specifically binding to a first epitope; (ii) a light chain constant domain of the first immunoglobulin (CL-1); (iii) a flexible peptide linker comprising the amino acid sequence (GGGGS)₃ (SEQ ID NO: 2506); and (iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ (SEQ ID NO: 2507) to form a single-chain variable fragment; (b) the second polypeptide comprises in the N-terminal to C-terminal direction: (i) a heavy chain variable domain of the first immunoglobulin (VH-1) that is capable of specifically binding to the first epitope; (ii) a first CH1 domain of the first immunoglobulin (CH1-1); and (iii) a first heterodimerization domain of the first immunoglobulin, wherein the first heterodimerization domain is incapable of forming a stable homodimer with another first heterodimerization domain; (c) the third polypeptide comprises in the N-terminal to C-terminal direction: (i) a heavy chain variable domain of a third immunoglobulin (VH-3) that is capable of specifically binding to the first epitope; (ii) a second CH1 domain of the third immunoglobulin (CH1-3); and (iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin; (d) the fourth polypeptide comprises in the N-terminal to C-terminal direction: (i) a light chain variable domain of the third immunoglobulin (VL-3) that is capable of specifically binding to the first epitope; and (ii) a light chain constant domain of the third immunoglobulin (CL-3); and wherein VL-2 comprises the CDR1 sequence, the CDR2 sequence and the CDR3 sequence of a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345; and/or wherein VH-2 comprises the CDR1 sequence, the CDR2 sequence and the CDR3 sequence of a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349. In some embodiments, both VH-1 and VH-3 comprise the CDR1 sequence, the CDR2 sequence and the CDR3 sequence of a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349; and/or both VL-1 and VL-3 comprise the CDR1 sequence, the CDR2 sequence and the CDR3 sequence of a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345.

In yet another aspect, the present disclosure provides a heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein: (a) the first polypeptide chain comprises in the N-terminal to C-terminal direction: (i) a light chain variable domain of a first immunoglobulin (VL-1) that is capable of specifically binding to a first epitope; (ii) a light chain constant domain of the first immunoglobulin (CL-1); (iii) a flexible peptide linker comprising the amino acid sequence (GGGGS)₃ (SEQ ID NO: 2506); and (iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ (SEQ ID NO: 2507) to form a single-chain variable fragment; (b) the second polypeptide comprises in the N-terminal to C-terminal direction: (i) a heavy chain variable domain of the first immunoglobulin (VH-1) that is capable of specifically binding to the first epitope; (ii) a first CH1 domain of the first immunoglobulin (CH1-1); and (iii) a first heterodimerization domain of the first immunoglobulin, wherein the first heterodimerization domain is incapable of forming a stable homodimer with another first heterodimerization domain; (c) the third polypeptide comprises in the N-terminal to C-terminal direction: (i) a heavy chain variable domain of a third immunoglobulin (VH-3) that is capable of specifically binding to a third epitope; (ii) a second CH1 domain of the third immunoglobulin (CH1-3); and (iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin; (d) the fourth polypeptide comprises in the N-terminal to C-terminal direction: (i) a light chain variable domain of the third immunoglobulin (VL-3) that is capable of specifically binding to the third epitope; and (ii) a light chain constant domain of the third immunoglobulin (CL-3); and wherein each of VL-1 and VL-3 independently comprises the CDR1 sequence, the CDR2 sequence and the CDR3 sequence of a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345; and/or wherein each of VH-1 and VH-3 independently comprises the CDR1 sequence, the CDR2 sequence and the CDR3 sequence of a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349; and/or wherein VL-2 comprises the CDR1 sequence, the CDR2 sequence and the CDR3 sequence of a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345; and/or wherein VH-2 comprises the CDR1 sequence, the CDR2 sequence and the CDR3 sequence of a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349.

Additionally or alternatively, in some embodiments of the heterodimeric multispecific antibodies disclosed herein, VH-1 or VH-3 comprises an amino acid sequence that is at least 80%, at least 85%, at least 90%, at least 95%, at least 99% or 100% identical to a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349; and/or the VL-1 or VL-3 comprises an amino acid sequence that is at least 80%, at least 85%, at least 90%, at least 95%, at least 99% or 100% identical to a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345.

Additionally or alternatively, in some embodiments of the heterodimeric multispecific antibodies disclosed herein, VH-2 or VH-4 comprises an amino acid sequence that is at least 80%, at least 85%, at least 90%, at least 95%, at least 99% or 100% identical to a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349; and/or VL-2 or VL-4 comprises an amino acid sequence that is at least 80%, at least 85%, at least 90%, at least 95%, at least 99%, or 100% identical to a VL amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345.

Additionally or alternatively, in some embodiments of the heterodimeric multispecific antibodies disclosed herein, each of VL-1 and VH-1 comprise a V_{L} amino acid sequence and a V_{H} amino acid sequence selected from the group consisting of SEQ ID NOs: 1 and 5 respectively; SEQ ID NOs: 9 and 13 respectively; SEQ ID NOs: 17 and 21 respectively; SEQ ID NOs: 25 and 29 respectively; SEQ ID NOs: 33 and 37 respectively; SEQ ID NOs: 41 and 45 respectively; SEQ ID NOs: 49 and 53 respectively; SEQ ID NOs: 57 and 61 respectively; SEQ ID NOs: 73 and 77 respectively; SEQ ID NOs: 89 and 93 respectively; SEQ ID NOs: 97 and 101 respectively; SEQ ID NOs: 105 and 109 respectively; SEQ ID NOs: 113 and 117 respectively; SEQ ID NOs: 121 and 125 respectively; SEQ ID NOs: 129 and 133 respectively; SEQ ID NOs: 145 and 149 respectively; SEQ ID NOs: 161 and 165 respectively; SEQ ID NOs: 169 and 173 respectively; SEQ ID NOs: 177 and 181 respectively; SEQ ID NOs: 193 and 197 respectively; SEQ ID NOs: 201 and 205 respectively; SEQ ID NOs: 233 and 237 respectively; SEQ ID NOs: 241 and 245 respectively; SEQ ID NOs: 257 and 261 respectively; SEQ ID NOs: 273 and 277 respectively; SEQ ID NOs: 281 and 285 respectively; SEQ ID NOs: 289 and 293 respectively; SEQ ID NOs: 297 and 301 respectively; SEQ ID NOs: 305 and 309 respectively; SEQ ID NOs: 313 and 317 respectively; SEQ ID NOs: 321 and 325 respectively; SEQ ID NOs: 329 and 333 respectively; SEQ ID NOs: 337 and 341 respectively; SEQ ID NOs: 345 and 349 respectively; SEQ ID NOs: 353 and 357 respectively; SEQ ID NOs: 361 and 365 respectively; SEQ ID NOs: 369 and 373 respectively; SEQ ID NOs: 377 and 381 respectively; SEQ ID NOs: 385 and 389 respectively; SEQ ID NOs: 393 and 397 respectively; SEQ ID NOs: 401 and 405 respectively; SEQ ID NOs: 409 and 413 respectively; SEQ ID NOs: 417 and 421 respectively; SEQ ID NOs: 425 and 429 respectively; SEQ ID NOs: 433 and 437 respectively; SEQ ID NOs: 441 and 445 respectively; SEQ ID NOs: 449 and 453 respectively; SEQ ID NOs: 457 and 461 respectively; SEQ ID NOs: 465 and 469 respectively; SEQ ID NOs: 481 and 485 respectively; SEQ ID NOs: 489 and 493 respectively; SEQ ID NOs: 497 and 501 respectively; SEQ ID NOs: 521 and 525 respectively; SEQ ID NOs: 529 and 533 respectively; SEQ ID NOs: 537 and 541 respectively; SEQ ID NOs: 545 and 549 respectively; SEQ ID NOs: 553 and 557 respectively; SEQ ID NOs: 561 and 565 respectively; SEQ ID NOs: 609 and 613 respectively; SEQ ID NOs: 617 and 621 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 729 and 733 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 769 and 773 respectively; SEQ ID NOs: 785 and 789 respectively; SEQ ID NOs: 793 and 797 respectively; SEQ ID NOs: 801 and 805 respectively; SEQ ID NOs: 809 and 813 respectively; SEQ ID NOs: 817 and 821 respectively; SEQ ID NOs: 825 and 829 respectively; SEQ ID NOs: 833 and 837 respectively; SEQ ID NOs: 841 and 845 respectively; SEQ ID NOs: 849 and 853 respectively; SEQ ID NOs: 857 and 861 respectively; SEQ ID NOs: 865 and 869 respectively; SEQ ID NOs: 873 and 877 respectively; SEQ ID NOs: 881 and 885 respectively; SEQ ID NOs: 889 and 893 respectively; SEQ ID NOs: 945 and 949 respectively; SEQ ID NOs: 953 and 957 respectively; SEQ ID NOs: 961 and 965 respectively; SEQ ID NOs: 977 and 981 respectively; SEQ ID NOs: 985 and 989 respectively; SEQ ID NOs: 993 and 997 respectively; SEQ ID NOs: 1001 and 1005 respectively; SEQ ID NOs: 1009 and 1013 respectively; SEQ ID NOs: 1017 and 1021 respectively; SEQ ID NOs: 1025 and 1029 respectively; SEQ ID NOs: 1033 and 1037 respectively; SEQ ID NOs: 1041 and 1045 respectively; SEQ ID NOs: 1065 and 1069 respectively; SEQ ID NOs: 1073 and 1077 respectively; SEQ ID NOs: 1081 and 1085 respectively; SEQ ID NOs: 1089 and 1093 respectively; SEQ ID NOs: 1097 and 1101 respectively; SEQ ID NOs: 1113 and 1117 respectively; SEQ ID NOs: 1121 and 1125 respectively; SEQ ID NOs: 1129 and 1133 respectively; SEQ ID NOs: 1137 and 1141 respectively; SEQ ID NOs: 1145 and 1149 respectively; SEQ ID NOs: 1153 and 1157 respectively; SEQ ID NOs: 1161 and 1165 respectively; SEQ ID NOs: 1169 and 1173 respectively; SEQ ID NOs: 1185 and 1189 respectively; SEQ ID NOs: 1193 and 1197 respectively; SEQ ID NOs: 1201 and 1205 respectively; SEQ ID NOs: 1209 and 1213 respectively; SEQ ID NOs: 1217 and 1221 respectively; SEQ ID NOs: 1225 and 1229 respectively; SEQ ID NOs: 1233 and 1237 respectively; SEQ ID NOs: 1241 and 1245 respectively; SEQ ID NOs: 1249 and 1253 respectively; SEQ ID NOs: 1257 and 1261 respectively; SEQ ID NOs: 1265 and 1269 respectively; SEQ ID NOs: 1273 and 1277 respectively; SEQ ID NOs: 1281 and 1285 respectively; SEQ ID NOs: 1289 and 1293 respectively; SEQ ID NOs: 1297 and 1301 respectively; SEQ ID NOs: 1305 and 1309 respectively; SEQ ID NOs: 1313 and 1317 respectively; SEQ ID NOs: 1321 and 1325 respectively; SEQ ID NOs: 1329 and 1333 respectively; SEQ ID NOs: 1337 and 1341 respectively; SEQ ID NOs: 1345 and 1349 respectively; SEQ ID NOs: 1353 and 1357 respectively; SEQ ID NOs: 1361 and 1365 respectively; SEQ ID NOs: 1369 and 1373 respectively; SEQ ID NOs: 1377 and 1381 respectively; SEQ ID NOs: 1385 and 1389 respectively; SEQ ID NOs: 1393 and 1397 respectively; SEQ ID NOs: 1401 and 1405 respectively; SEQ ID NOs: 1409 and 1413 respectively; SEQ ID NOs: 1417 and 1421 respectively; SEQ ID NOs: 1433 and 1437 respectively; SEQ ID NOs: 1441 and 1445 respectively; SEQ ID NOs: 1457 and 1461 respectively; SEQ ID NOs: 1465 and 1469 respectively; SEQ ID NOs: 1473 and 1477 respectively; SEQ ID NOs: 1481 and 1485 respectively; SEQ ID NOs: 1489 and 1493 respectively; SEQ ID NOs: 1497 and 1501 respectively; SEQ ID NOs: 1505 and 1509 respectively; SEQ ID NOs: 1513 and 1517 respectively; SEQ ID NOs: 1521 and 1525 respectively; SEQ ID NOs: 1529 and 1533 respectively; SEQ ID NOs: 1545 and 1549 respectively; SEQ ID NOs: 1553 and 1557 respectively; SEQ ID NOs: 1561 and 1565 respectively; SEQ ID NOs: 1569 and 1573 respectively; SEQ ID NOs: 1577 and 1581 respectively; SEQ ID NOs: 1585 and 1589 respectively; SEQ ID NOs: 1593 and 1597 respectively; SEQ ID NOs: 1601 and 1605 respectively; SEQ ID NOs: 1609 and 1613 respectively; SEQ ID NOs: 1617 and 1621 respectively; SEQ ID NOs: 1625 and 1629 respectively; SEQ ID NOs: 1633 and 1637 respectively; SEQ ID NOs: 1649 and 1653 respectively; SEQ ID NOs: 1657 and 1661 respectively; SEQ ID NOs: 1673 and 1677 respectively; SEQ ID NOs: 1681 and 1685 respectively; SEQ ID NOs: 1689 and 1693 respectively; SEQ ID NOs: 1697 and 1701 respectively; SEQ ID NOs: 1705 and 1709 respectively; SEQ ID NOs: 1713 and 1717 respectively; SEQ ID NOs: 1721 and 1725 respectively; SEQ ID NOs: 1729 and 1733 respectively; SEQ ID NOs: 1737 and 1741 respectively; SEQ ID NOs: 1745 and 1749 respectively; SEQ ID NOs: 1753 and 1757 respectively; SEQ ID NOs: 1761 and 1765 respectively; SEQ ID NOs: 1769 and 1773 respectively; SEQ ID NOs: 1777 and 1781 respectively; SEQ ID NOs: 1785 and 1789 respectively; SEQ ID NOs: 1793 and 1797 respectively; SEQ ID NOs: 1801 and 1805 respectively; SEQ ID NOs: 1809 and 1813 respectively; SEQ ID NOs: 1817 and 1821 respectively; SEQ ID NOs: 1833 and 1837 respectively; SEQ ID NOs: 1841 and 1845 respectively; SEQ ID NOs: 1849 and 1853 respectively; SEQ ID NOs: 1857 and 1861 respectively; SEQ ID NOs: 1865 and 1869 respectively; SEQ ID NOs: 1873 and 1877 respectively; SEQ ID NOs: 1881 and 1885 respectively; SEQ ID NOs: 1889 and 1893 respectively; SEQ ID NOs: 1913 and 1917 respectively; SEQ ID NOs: 1937 and 1941 respectively; SEQ ID NOs: 1945 and 1949 respectively; SEQ ID NOs: 1953 and 1957 respectively; SEQ ID NOs: 1961 and 1965 respectively; SEQ ID NOs: 1969 and 1973 respectively; SEQ ID NOs: 1977 and 1981 respectively; SEQ ID NOs: 1985 and 1989 respectively; SEQ ID NOs: 1993 and 1997 respectively; SEQ ID NOs: 2001 and 2005 respectively; SEQ ID NOs: 2009 and 2013 respectively; SEQ ID NOs: 2017 and 2021 respectively; SEQ ID NOs: 2025 and 2029 respectively; SEQ ID NOs: 2033 and 2037 respectively; SEQ ID NOs: 2041 and 2045 respectively; SEQ ID NOs: 2049 and 2053 respectively; SEQ ID NOs: 2057 and 2061 respectively; SEQ ID NOs: 2065 and 2069 respectively; SEQ ID NOs: 2073 and 2077 respectively; SEQ ID NOs: 2081 and 2085 respectively; SEQ ID NOs: 2089 and 2093 respectively; SEQ ID NOs: 2097 and 2101 respectively; SEQ ID NOs: 2105 and 2109 respectively; SEQ ID NOs: 2113 and 2117 respectively; SEQ ID NOs: 2121 and 2125 respectively; SEQ ID NOs: 2129 and 2133 respectively; SEQ ID NOs: 2137 and 2141 respectively; SEQ ID NOs: 2145 and 2149 respectively; SEQ ID NOs: 2153 and 2157 respectively; SEQ ID NOs: 2161 and 2165 respectively; SEQ ID NOs: 2169 and 2173 respectively; SEQ ID NOs: 2177 and 2181 respectively; SEQ ID NOs: 2185 and 2189 respectively; SEQ ID NOs: 2193 and 2197 respectively; SEQ ID NOs: 2201 and 2205 respectively; SEQ ID NOs: 2209 and 2213 respectively; SEQ ID NOs: 2217 and 2221 respectively; SEQ ID NOs: 2225 and 2229 respectively; SEQ ID NOs: 2233 and 2237 respectively; SEQ ID NOs: 2241 and 2245 respectively; SEQ ID NOs: 2249 and 2253 respectively; SEQ ID NOs: 2257 and 2261 respectively; SEQ ID NOs: 2273 and 2277 respectively; SEQ ID NOs: 2281 and 2285 respectively; SEQ ID NOs: 2305 and 2309 respectively; SEQ ID NOs: 2313 and 2317 respectively; SEQ ID NOs: 2321 and 2325 respectively; SEQ ID NOs: 2329 and 2333 respectively; SEQ ID NOs: 2337 and 2341 respectively; and SEQ ID NOs: 2345 and 2349 respectively.

Additionally or alternatively, in some embodiments of the heterodimeric multispecific antibodies disclosed herein, each of VL-3 and VH-3 comprise a V_{L} amino acid sequence and a V_{H} amino acid sequence selected from the group consisting of SEQ ID NOs: 1 and 5 respectively; SEQ ID NOs: 9 and 13 respectively; SEQ ID NOs: 17 and 21 respectively; SEQ ID NOs: 25 and 29 respectively; SEQ ID NOs: 33 and 37 respectively; SEQ ID NOs: 41 and 45 respectively; SEQ ID NOs: 49 and 53 respectively; SEQ ID NOs: 57 and 61 respectively; SEQ ID NOs: 73 and 77 respectively; SEQ ID NOs: 89 and 93 respectively; SEQ ID NOs: 97 and 101 respectively; SEQ ID NOs: 105 and 109 respectively; SEQ ID NOs: 113 and 117 respectively; SEQ ID NOs: 121 and 125 respectively; SEQ ID NOs: 129 and 133 respectively; SEQ ID NOs: 145 and 149 respectively; SEQ ID NOs: 161 and 165 respectively; SEQ ID NOs: 169 and 173 respectively; SEQ ID NOs: 177 and 181 respectively; SEQ ID NOs: 193 and 197 respectively; SEQ ID NOs: 201 and 205 respectively; SEQ ID NOs: 233 and 237 respectively; SEQ ID NOs: 241 and 245 respectively; SEQ ID NOs: 257 and 261 respectively; SEQ ID NOs: 273 and 277 respectively; SEQ ID NOs: 281 and 285 respectively; SEQ ID NOs: 289 and 293 respectively; SEQ ID NOs: 297 and 301 respectively; SEQ ID NOs: 305 and 309 respectively; SEQ ID NOs: 313 and 317 respectively; SEQ ID NOs: 321 and 325 respectively; SEQ ID NOs: 329 and 333 respectively; SEQ ID NOs: 337 and 341 respectively; SEQ ID NOs: 345 and 349 respectively; SEQ ID NOs: 353 and 357 respectively; SEQ ID NOs: 361 and 365 respectively; SEQ ID NOs: 369 and 373 respectively; SEQ ID NOs: 377 and 381 respectively; SEQ ID NOs: 385 and 389 respectively; SEQ ID NOs: 393 and 397 respectively; SEQ ID NOs: 401 and 405 respectively; SEQ ID NOs: 409 and 413 respectively; SEQ ID NOs: 417 and 421 respectively; SEQ ID NOs: 425 and 429 respectively; SEQ ID NOs: 433 and 437 respectively; SEQ ID NOs: 441 and 445 respectively; SEQ ID NOs: 449 and 453 respectively; SEQ ID NOs: 457 and 461 respectively; SEQ ID NOs: 465 and 469 respectively; SEQ ID NOs: 481 and 485 respectively; SEQ ID NOs: 489 and 493 respectively; SEQ ID NOs: 497 and 501 respectively; SEQ ID NOs: 521 and 525 respectively; SEQ ID NOs: 529 and 533 respectively; SEQ ID NOs: 537 and 541 respectively; SEQ ID NOs: 545 and 549 respectively; SEQ ID NOs: 553 and 557 respectively; SEQ ID NOs: 561 and 565 respectively; SEQ ID NOs: 609 and 613 respectively; SEQ ID NOs: 617 and 621 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 729 and 733 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 769 and 773 respectively; SEQ ID NOs: 785 and 789 respectively; SEQ ID NOs: 793 and 797 respectively; SEQ ID NOs: 801 and 805 respectively; SEQ ID NOs: 809 and 813 respectively; SEQ ID NOs: 817 and 821 respectively; SEQ ID NOs: 825 and 829 respectively; SEQ ID NOs: 833 and 837 respectively; SEQ ID NOs: 841 and 845 respectively; SEQ ID NOs: 849 and 853 respectively; SEQ ID NOs: 857 and 861 respectively; SEQ ID NOs: 865 and 869 respectively; SEQ ID NOs: 873 and 877 respectively; SEQ ID NOs: 881 and 885 respectively; SEQ ID NOs: 889 and 893 respectively; SEQ ID NOs: 945 and 949 respectively; SEQ ID NOs: 953 and 957 respectively; SEQ ID NOs: 961 and 965 respectively; SEQ ID NOs: 977 and 981 respectively; SEQ ID NOs: 985 and 989 respectively; SEQ ID NOs: 993 and 997 respectively; SEQ ID NOs: 1001 and 1005 respectively; SEQ ID NOs: 1009 and 1013 respectively; SEQ ID NOs: 1017 and 1021 respectively; SEQ ID NOs: 1025 and 1029 respectively; SEQ ID NOs: 1033 and 1037 respectively; SEQ ID NOs: 1041 and 1045 respectively; SEQ ID NOs: 1065 and 1069 respectively; SEQ ID NOs: 1073 and 1077 respectively; SEQ ID NOs: 1081 and 1085 respectively; SEQ ID NOs: 1089 and 1093 respectively; SEQ ID NOs: 1097 and 1101 respectively; SEQ ID NOs: 1113 and 1117 respectively; SEQ ID NOs: 1121 and 1125 respectively; SEQ ID NOs: 1129 and 1133 respectively; SEQ ID NOs: 1137 and 1141 respectively; SEQ ID NOs: 1145 and 1149 respectively; SEQ ID NOs: 1153 and 1157 respectively; SEQ ID NOs: 1161 and 1165 respectively; SEQ ID NOs: 1169 and 1173 respectively; SEQ ID NOs: 1185 and 1189 respectively; SEQ ID NOs: 1193 and 1197 respectively; SEQ ID NOs: 1201 and 1205 respectively; SEQ ID NOs: 1209 and 1213 respectively; SEQ ID NOs: 1217 and 1221 respectively; SEQ ID NOs: 1225 and 1229 respectively; SEQ ID NOs: 1233 and 1237 respectively; SEQ ID NOs: 1241 and 1245 respectively; SEQ ID NOs: 1249 and 1253 respectively; SEQ ID NOs: 1257 and 1261 respectively; SEQ ID NOs: 1265 and 1269 respectively; SEQ ID NOs: 1273 and 1277 respectively; SEQ ID NOs: 1281 and 1285 respectively; SEQ ID NOs: 1289 and 1293 respectively; SEQ ID NOs: 1297 and 1301 respectively; SEQ ID NOs: 1305 and 1309 respectively; SEQ ID NOs: 1313 and 1317 respectively; SEQ ID NOs: 1321 and 1325 respectively; SEQ ID NOs: 1329 and 1333 respectively; SEQ ID NOs: 1337 and 1341 respectively; SEQ ID NOs: 1345 and 1349 respectively; SEQ ID NOs: 1353 and 1357 respectively; SEQ ID NOs: 1361 and 1365 respectively; SEQ ID NOs: 1369 and 1373 respectively; SEQ ID NOs: 1377 and 1381 respectively; SEQ ID NOs: 1385 and 1389 respectively; SEQ ID NOs: 1393 and 1397 respectively; SEQ ID NOs: 1401 and 1405 respectively; SEQ ID NOs: 1409 and 1413 respectively; SEQ ID NOs: 1417 and 1421 respectively; SEQ ID NOs: 1433 and 1437 respectively; SEQ ID NOs: 1441 and 1445 respectively; SEQ ID NOs: 1457 and 1461 respectively; SEQ ID NOs: 1465 and 1469 respectively; SEQ ID NOs: 1473 and 1477 respectively; SEQ ID NOs: 1481 and 1485 respectively; SEQ ID NOs: 1489 and 1493 respectively; SEQ ID NOs: 1497 and 1501 respectively; SEQ ID NOs: 1505 and 1509 respectively; SEQ ID NOs: 1513 and 1517 respectively; SEQ ID NOs: 1521 and 1525 respectively; SEQ ID NOs: 1529 and 1533 respectively; SEQ ID NOs: 1545 and 1549 respectively; SEQ ID NOs: 1553 and 1557 respectively; SEQ ID NOs: 1561 and 1565 respectively; SEQ ID NOs: 1569 and 1573 respectively; SEQ ID NOs: 1577 and 1581 respectively; SEQ ID NOs: 1585 and 1589 respectively; SEQ ID NOs: 1593 and 1597 respectively; SEQ ID NOs: 1601 and 1605 respectively; SEQ ID NOs: 1609 and 1613 respectively; SEQ ID NOs: 1617 and 1621 respectively; SEQ ID NOs: 1625 and 1629 respectively; SEQ ID NOs: 1633 and 1637 respectively; SEQ ID NOs: 1649 and 1653 respectively; SEQ ID NOs: 1657 and 1661 respectively; SEQ ID NOs: 1673 and 1677 respectively; SEQ ID NOs: 1681 and 1685 respectively; SEQ ID NOs: 1689 and 1693 respectively; SEQ ID NOs: 1697 and 1701 respectively; SEQ ID NOs: 1705 and 1709 respectively; SEQ ID NOs: 1713 and 1717 respectively; SEQ ID NOs: 1721 and 1725 respectively; SEQ ID NOs: 1729 and 1733 respectively; SEQ ID NOs: 1737 and 1741 respectively; SEQ ID NOs: 1745 and 1749 respectively; SEQ ID NOs: 1753 and 1757 respectively; SEQ ID NOs: 1761 and 1765 respectively; SEQ ID NOs: 1769 and 1773 respectively; SEQ ID NOs: 1777 and 1781 respectively; SEQ ID NOs: 1785 and 1789 respectively; SEQ ID NOs: 1793 and 1797 respectively; SEQ ID NOs: 1801 and 1805 respectively; SEQ ID NOs: 1809 and 1813 respectively; SEQ ID NOs: 1817 and 1821 respectively; SEQ ID NOs: 1833 and 1837 respectively; SEQ ID NOs: 1841 and 1845 respectively; SEQ ID NOs: 1849 and 1853 respectively; SEQ ID NOs: 1857 and 1861 respectively; SEQ ID NOs: 1865 and 1869 respectively; SEQ ID NOs: 1873 and 1877 respectively; SEQ ID NOs: 1881 and 1885 respectively; SEQ ID NOs: 1889 and 1893 respectively; SEQ ID NOs: 1913 and 1917 respectively; SEQ ID NOs: 1937 and 1941 respectively; SEQ ID NOs: 1945 and 1949 respectively; SEQ ID NOs: 1953 and 1957 respectively; SEQ ID NOs: 1961 and 1965 respectively; SEQ ID NOs: 1969 and 1973 respectively; SEQ ID NOs: 1977 and 1981 respectively; SEQ ID NOs: 1985 and 1989 respectively; SEQ ID NOs: 1993 and 1997 respectively; SEQ ID NOs: 2001 and 2005 respectively; SEQ ID NOs: 2009 and 2013 respectively; SEQ ID NOs: 2017 and 2021 respectively; SEQ ID NOs: 2025 and 2029 respectively; SEQ ID NOs: 2033 and 2037 respectively; SEQ ID NOs: 2041 and 2045 respectively; SEQ ID NOs: 2049 and 2053 respectively; SEQ ID NOs: 2057 and 2061 respectively; SEQ ID NOs: 2065 and 2069 respectively; SEQ ID NOs: 2073 and 2077 respectively; SEQ ID NOs: 2081 and 2085 respectively; SEQ ID NOs: 2089 and 2093 respectively; SEQ ID NOs: 2097 and 2101 respectively; SEQ ID NOs: 2105 and 2109 respectively; SEQ ID NOs: 2113 and 2117 respectively; SEQ ID NOs: 2121 and 2125 respectively; SEQ ID NOs: 2129 and 2133 respectively; SEQ ID NOs: 2137 and 2141 respectively; SEQ ID NOs: 2145 and 2149 respectively; SEQ ID NOs: 2153 and 2157 respectively; SEQ ID NOs: 2161 and 2165 respectively; SEQ ID NOs: 2169 and 2173 respectively; SEQ ID NOs: 2177 and 2181 respectively; SEQ ID NOs: 2185 and 2189 respectively; SEQ ID NOs: 2193 and 2197 respectively; SEQ ID NOs: 2201 and 2205 respectively; SEQ ID NOs: 2209 and 2213 respectively; SEQ ID NOs: 2217 and 2221 respectively; SEQ ID NOs: 2225 and 2229 respectively; SEQ ID NOs: 2233 and 2237 respectively; SEQ ID NOs: 2241 and 2245 respectively; SEQ ID NOs: 2249 and 2253 respectively; SEQ ID NOs: 2257 and 2261 respectively; SEQ ID NOs: 2273 and 2277 respectively; SEQ ID NOs: 2281 and 2285 respectively; SEQ ID NOs: 2305 and 2309 respectively; SEQ ID NOs: 2313 and 2317 respectively; SEQ ID NOs: 2321 and 2325 respectively; SEQ ID NOs: 2329 and 2333 respectively; SEQ ID NOs: 2337 and 2341 respectively; and SEQ ID NOs: 2345 and 2349 respectively.

Additionally or alternatively, in some embodiments of the heterodimeric multispecific antibodies disclosed herein, each of VL-1 and VH-1 comprise a V_{L} amino acid sequence and a V_{H} amino acid sequence selected from the group consisting of SEQ ID NOs: 9 and 13 respectively; SEQ ID NOs: 49 and 53 respectively; SEQ ID NOs: 57 and 61 respectively; SEQ ID NOs: 65 and 69 respectively; SEQ ID NOs: 81 and 85 respectively; SEQ ID NOs: 153 and 157 respectively; SEQ ID NOs: 161 and 165 respectively; SEQ ID NOs: 193 and 197 respectively; SEQ ID NOs: 201 and 205 respectively; SEQ ID NOs: 273 and 277 respectively; SEQ ID NOs: 281 and 285 respectively; SEQ ID NOs: 289 and 293 respectively; SEQ ID NOs: 297 and 301 respectively; SEQ ID NOs: 305 and 309 respectively; SEQ ID NOs: 313 and 317 respectively; SEQ ID NOs: 361 and 365 respectively; SEQ ID NOs: 377 and 381 respectively; SEQ ID NOs: 393 and 397 respectively; SEQ ID NOs: 401 and 405 respectively; SEQ ID NOs: 409 and 413 respectively; SEQ ID NOs: 417 and 421 respectively; SEQ ID NOs: 425 and 429 respectively; SEQ ID NOs: 433 and 437 respectively; SEQ ID NOs: 441 and 445 respectively; SEQ ID NOs: 449 and 453 respectively; SEQ ID NOs: 457 and 461 respectively; SEQ ID NOs: 465 and 469 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 777 and 781 respectively; SEQ ID NOs: 825 and 829 respectively; SEQ ID NOs: 833 and 837 respectively; SEQ ID NOs: 841 and 845 respectively; SEQ ID NOs: 953 and 957 respectively; SEQ ID NOs: 961 and 965 respectively; SEQ ID NOs: 977 and 981 respectively; SEQ ID NOs: 993 and 997 respectively; SEQ ID NOs: 1001 and 1005 respectively; SEQ ID NOs: 1009 and 1013 respectively; SEQ ID NOs: 1017 and 1021 respectively; SEQ ID NOs: 1033 and 1037 respectively; SEQ ID NOs: 1049 and 1053 respectively; SEQ ID NOs: 1073 and 1077 respectively; SEQ ID NOs: 1081 and 1085 respectively; SEQ ID NOs: 1089 and 1093 respectively; SEQ ID NOs: 1105 and 1109 respectively; SEQ ID NOs: 1129 and 1133 respectively; SEQ ID NOs: 1137 and 1141 respectively; SEQ ID NOs: 1153 and 1157 respectively; SEQ ID NOs: 1161 and 1165 respectively; SEQ ID NOs: 1177 and 1181 respectively; SEQ ID NOs: 1225 and 1229 respectively; SEQ ID NOs: 1241 and 1245 respectively; SEQ ID NOs: 1257 and 1261 respectively; SEQ ID NOs: 1265 and 1269 respectively; SEQ ID NOs: 1297 and 1301 respectively; SEQ ID NOs: 1393 and 1397 respectively; SEQ ID NOs: 1409 and 1413 respectively; SEQ ID NOs: 1425 and 1429 respectively; SEQ ID NOs: 1441 and 1445 respectively; SEQ ID NOs: 1449 and 1453 respectively; SEQ ID NOs: 1457 and 1461 respectively; SEQ ID NOs: 1465 and 1469 respectively; SEQ ID NOs: 1473 and 1477 respectively; SEQ ID NOs: 1481 and 1485 respectively; SEQ ID NOs: 1497 and 1501 respectively; SEQ ID NOs: 1505 and 1509 respectively; SEQ ID NOs: 1513 and 1517 respectively; SEQ ID NOs: 1521 and 1525 respectively; SEQ ID NOs: 1529 and 1533 respectively; SEQ ID NOs: 1545 and 1549 respectively; SEQ ID NOs: 1553 and 1557 respectively; SEQ ID NOs: 1561 and 1565 respectively; SEQ ID NOs: 1569 and 1573 respectively; SEQ ID NOs: 1577 and 1581 respectively; SEQ ID NOs: 1585 and 1589 respectively; SEQ ID NOs: 1609 and 1613 respectively; SEQ ID NOs: 1617 and 1621 respectively; SEQ ID NOs: 1649 and 1653 respectively; SEQ ID NOs: 1657 and 1661 respectively; SEQ ID NOs: 1673 and 1677 respectively; SEQ ID NOs: 1689 and 1693 respectively; SEQ ID NOs: 1697 and 1701 respectively; SEQ ID NOs: 1705 and 1709 respectively; SEQ ID NOs: 1713 and 1717 respectively; SEQ ID NOs: 1721 and 1725 respectively; SEQ ID NOs: 1729 and 1733 respectively; SEQ ID NOs: 1745 and 1749 respectively; SEQ ID NOs: 1753 and 1757 respectively; SEQ ID NOs: 1761 and 1765 respectively; SEQ ID NOs: 1769 and 1773 respectively; SEQ ID NOs: 1777 and 1781 respectively; SEQ ID NOs: 1785 and 1789 respectively; SEQ ID NOs: 1793 and 1797 respectively; SEQ ID NOs: 1817 and 1821 respectively; SEQ ID NOs: 1833 and 1837 respectively; SEQ ID NOs: 1841 and 1845 respectively; SEQ ID NOs: 1849 and 1853 respectively; SEQ ID NOs: 1857 and 1861 respectively; SEQ ID NOs: 1865 and 1869 respectively; SEQ ID NOs: 1889 and 1893 respectively; SEQ ID NOs: 2257 and 2261 respectively; SEQ ID NOs: 2265 and 2269 respectively; SEQ ID NOs: 2281 and 2285 respectively; SEQ ID NOs: 2297 and 2301 respectively; SEQ ID NOs: 2305 and 2309 respectively; SEQ ID NOs: 2313 and 2317 respectively; SEQ ID NOs: 2321 and 2325 respectively; SEQ ID NOs: 2329 and 2333 respectively; SEQ ID NOs: 2337 and 2341 respectively; and SEQ ID NOs: 2345 and 2349 respectively.

Additionally or alternatively, in some embodiments of the heterodimeric multispecific antibodies disclosed herein, each of VL-3 and VH-3 comprise a V_{L} amino acid sequence and a V_{H} amino acid sequence selected from the group consisting of SEQ ID NOs: 9 and 13 respectively; SEQ ID NOs: 49 and 53 respectively; SEQ ID NOs: 57 and 61 respectively; SEQ ID NOs: 65 and 69 respectively; SEQ ID NOs: 81 and 85 respectively; SEQ ID NOs: 153 and 157 respectively; SEQ ID NOs: 161 and 165 respectively; SEQ ID NOs: 193 and 197 respectively; SEQ ID NOs: 201 and 205 respectively; SEQ ID NOs: 273 and 277 respectively; SEQ ID NOs: 281 and 285 respectively; SEQ ID NOs: 289 and 293 respectively; SEQ ID NOs: 297 and 301 respectively; SEQ ID NOs: 305 and 309 respectively; SEQ ID NOs: 313 and 317 respectively; SEQ ID NOs: 361 and 365 respectively; SEQ ID NOs: 377 and 381 respectively; SEQ ID NOs: 393 and 397 respectively; SEQ ID NOs: 401 and 405 respectively; SEQ ID NOs: 409 and 413 respectively; SEQ ID NOs: 417 and 421 respectively; SEQ ID NOs: 425 and 429 respectively; SEQ ID NOs: 433 and 437 respectively; SEQ ID NOs: 441 and 445 respectively; SEQ ID NOs: 449 and 453 respectively; SEQ ID NOs: 457 and 461 respectively; SEQ ID NOs: 465 and 469 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 777 and 781 respectively; SEQ ID NOs: 825 and 829 respectively; SEQ ID NOs: 833 and 837 respectively; SEQ ID NOs: 841 and 845 respectively; SEQ ID NOs: 953 and 957 respectively; SEQ ID NOs: 961 and 965 respectively; SEQ ID NOs: 977 and 981 respectively; SEQ ID NOs: 993 and 997 respectively; SEQ ID NOs: 1001 and 1005 respectively; SEQ ID NOs: 1009 and 1013 respectively; SEQ ID NOs: 1017 and 1021 respectively; SEQ ID NOs: 1033 and 1037 respectively; SEQ ID NOs: 1049 and 1053 respectively; SEQ ID NOs: 1073 and 1077 respectively; SEQ ID NOs: 1081 and 1085 respectively; SEQ ID NOs: 1089 and 1093 respectively; SEQ ID NOs: 1105 and 1109 respectively; SEQ ID NOs: 1129 and 1133 respectively; SEQ ID NOs: 1137 and 1141 respectively; SEQ ID NOs: 1153 and 1157 respectively; SEQ ID NOs: 1161 and 1165 respectively; SEQ ID NOs: 1177 and 1181 respectively; SEQ ID NOs: 1225 and 1229 respectively; SEQ ID NOs: 1241 and 1245 respectively; SEQ ID NOs: 1257 and 1261 respectively; SEQ ID NOs: 1265 and 1269 respectively; SEQ ID NOs: 1297 and 1301 respectively; SEQ ID NOs: 1393 and 1397 respectively; SEQ ID NOs: 1409 and 1413 respectively; SEQ ID NOs: 1425 and 1429 respectively; SEQ ID NOs: 1441 and 1445 respectively; SEQ ID NOs: 1449 and 1453 respectively; SEQ ID NOs: 1457 and 1461 respectively; SEQ ID NOs: 1465 and 1469 respectively; SEQ ID NOs: 1473 and 1477 respectively; SEQ ID NOs: 1481 and 1485 respectively; SEQ ID NOs: 1497 and 1501 respectively; SEQ ID NOs: 1505 and 1509 respectively; SEQ ID NOs: 1513 and 1517 respectively; SEQ ID NOs: 1521 and 1525 respectively; SEQ ID NOs: 1529 and 1533 respectively; SEQ ID NOs: 1545 and 1549 respectively; SEQ ID NOs: 1553 and 1557 respectively; SEQ ID NOs: 1561 and 1565 respectively; SEQ ID NOs: 1569 and 1573 respectively; SEQ ID NOs: 1577 and 1581 respectively; SEQ ID NOs: 1585 and 1589 respectively; SEQ ID NOs: 1609 and 1613 respectively; SEQ ID NOs: 1617 and 1621 respectively; SEQ ID NOs: 1649 and 1653 respectively; SEQ ID NOs: 1657 and 1661 respectively; SEQ ID NOs: 1673 and 1677 respectively; SEQ ID NOs: 1689 and 1693 respectively; SEQ ID NOs: 1697 and 1701 respectively; SEQ ID NOs: 1705 and 1709 respectively; SEQ ID NOs: 1713 and 1717 respectively; SEQ ID NOs: 1721 and 1725 respectively; SEQ ID NOs: 1729 and 1733 respectively; SEQ ID NOs: 1745 and 1749 respectively; SEQ ID NOs: 1753 and 1757 respectively; SEQ ID NOs: 1761 and 1765 respectively; SEQ ID NOs: 1769 and 1773 respectively; SEQ ID NOs: 1777 and 1781 respectively; SEQ ID NOs: 1785 and 1789 respectively; SEQ ID NOs: 1793 and 1797 respectively; SEQ ID NOs: 1817 and 1821 respectively; SEQ ID NOs: 1833 and 1837 respectively; SEQ ID NOs: 1841 and 1845 respectively; SEQ ID NOs: 1849 and 1853 respectively; SEQ ID NOs: 1857 and 1861 respectively; SEQ ID NOs: 1865 and 1869 respectively; SEQ ID NOs: 1889 and 1893 respectively; SEQ ID NOs: 2257 and 2261 respectively; SEQ ID NOs: 2265 and 2269 respectively; SEQ ID NOs: 2281 and 2285 respectively; SEQ ID NOs: 2297 and 2301 respectively; SEQ ID NOs: 2305 and 2309 respectively; SEQ ID NOs: 2313 and 2317 respectively; SEQ ID NOs: 2321 and 2325 respectively; SEQ ID NOs: 2329 and 2333 respectively; SEQ ID NOs: 2337 and 2341 respectively; and SEQ ID NOs: 2345 and 2349 respectively.

Additionally or alternatively, in some embodiments of the heterodimeric multispecific antibodies disclosed herein, each of VL-2 and VH-2 comprise a V_{L} amino acid sequence and a V_{H} amino acid sequence selected from the group consisting of SEQ ID NOs: 17 and 21 respectively; SEQ ID NOs: 25 and 29 respectively; SEQ ID NOs: 33 and 37 respectively; SEQ ID NOs: 41 and 45 respectively; SEQ ID NOs: 121 and 125 respectively; SEQ ID NOs: 137 and 141 respectively; SEQ ID NOs: 169 and 173 respectively; SEQ ID NOs: 177 and 181 respectively; SEQ ID NOs: 185 and 189 respectively; SEQ ID NOs: 193 and 197 respectively; SEQ ID NOs: 201 and 205 respectively; SEQ ID NOs: 209 and 213 respectively; SEQ ID NOs: 217 and 221 respectively; SEQ ID NOs: 225 and 229 respectively; SEQ ID NOs: 233 and 237 respectively; SEQ ID NOs: 241 and 245 respectively; SEQ ID NOs: 249 and 253 respectively; SEQ ID NOs: 257 and 261 respectively; SEQ ID NOs: 265 and 269 respectively; SEQ ID NOs: 321 and 325 respectively; SEQ ID NOs: 329 and 333 respectively; SEQ ID NOs: 337 and 341 respectively; SEQ ID NOs: 393 and 397 respectively; SEQ ID NOs: 401 and 405 respectively; SEQ ID NOs: 409 and 413 respectively; SEQ ID NOs: 473 and 477 respectively; SEQ ID NOs: 481 and 485 respectively; SEQ ID NOs: 489 and 493 respectively; SEQ ID NOs: 497 and 501 respectively; SEQ ID NOs: 505 and 509 respectively; SEQ ID NOs: 513 and 517 respectively; SEQ ID NOs: 545 and 549 respectively; SEQ ID NOs: 553 and 557 respectively; SEQ ID NOs: 561 and 565 respectively; SEQ ID NOs: 569 and 573 respectively; SEQ ID NOs: 577 and 581 respectively; SEQ ID NOs: 585 and 589 respectively; SEQ ID NOs: 593 and 597 respectively; SEQ ID NOs: 601 and 605 respectively; SEQ ID NOs: 625 and 629 respectively; SEQ ID NOs: 633 and 637 respectively; SEQ ID NOs: 641 and 645 respectively; SEQ ID NOs: 649 and 653 respectively; SEQ ID NOs: 657 and 661 respectively; SEQ ID NOs: 665 and 669 respectively; SEQ ID NOs: 673 and 677 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 729 and 733 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 769 and 773 respectively; SEQ ID NOs: 785 and 789 respectively; SEQ ID NOs: 793 and 797 respectively; SEQ ID NOs: 801 and 805 respectively; SEQ ID NOs: 809 and 813 respectively; SEQ ID NOs: 817 and 821 respectively; SEQ ID NOs: 849 and 853 respectively; SEQ ID NOs: 857 and 861 respectively; SEQ ID NOs: 865 and 869 respectively; SEQ ID NOs: 873 and 877 respectively; SEQ ID NOs: 881 and 885 respectively; SEQ ID NOs: 889 and 893 respectively; SEQ ID NOs: 897 and 901 respectively; SEQ ID NOs: 905 and 909 respectively; SEQ ID NOs: 913 and 917 respectively; SEQ ID NOs: 921 and 925 respectively; SEQ ID NOs: 929 and 933 respectively; SEQ ID NOs: 937 and 941 respectively; SEQ ID NOs: 945 and 949 respectively; SEQ ID NOs: 969 and 973 respectively; SEQ ID NOs: 977 and 981 respectively; SEQ ID NOs: 1009 and 1013 respectively; SEQ ID NOs: 1057 and 1061 respectively; SEQ ID NOs: 1537 and 1541 respectively; SEQ ID NOs: 1569 and 1573 respectively; SEQ ID NOs: 1601 and 1605 respectively; SEQ ID NOs: 1641 and 1645 respectively; SEQ ID NOs: 1665 and 1669 respectively; SEQ ID NOs: 1825 and 1829 respectively; SEQ ID NOs: 1865 and 1869 respectively; SEQ ID NOs: 1897 and 1901 respectively; SEQ ID NOs: 1905 and 1909 respectively; SEQ ID NOs: 1913 and 1917 respectively; SEQ ID NOs: 1921 and 1925 respectively; SEQ ID NOs: 1929 and 1933 respectively; SEQ ID NOs: 2265 and 2269 respectively; SEQ ID NOs: 2281 and 2285 respectively; 2289 and 2293 respectively; 2329 and 2333 respectively; and SEQ ID NOs: 2345 and 2349, respectively.

Additionally or alternatively, in some embodiments of the heterodimeric multispecific antibodies disclosed herein, each of VL-4 and VH-4 comprise a V_{L} amino acid sequence and a V_{H} amino acid sequence selected from the group consisting of SEQ ID NOs: 17 and 21 respectively; SEQ ID NOs: 25 and 29 respectively; SEQ ID NOs: 33 and 37 respectively; SEQ ID NOs: 41 and 45 respectively; SEQ ID NOs: 121 and 125 respectively; SEQ ID NOs: 137 and 141 respectively; SEQ ID NOs: 169 and 173 respectively; SEQ ID NOs: 177 and 181 respectively; SEQ ID NOs: 185 and 189 respectively; SEQ ID NOs: 193 and 197 respectively; SEQ ID NOs: 201 and 205 respectively; SEQ ID NOs: 209 and 213 respectively; SEQ ID NOs: 217 and 221 respectively; SEQ ID NOs: 225 and 229 respectively; SEQ ID NOs: 233 and 237 respectively; SEQ ID NOs: 241 and 245 respectively; SEQ ID NOs: 249 and 253 respectively; SEQ ID NOs: 257 and 261 respectively; SEQ ID NOs: 265 and 269 respectively; SEQ ID NOs: 321 and 325 respectively; SEQ ID NOs: 329 and 333 respectively; SEQ ID NOs: 337 and 341 respectively; SEQ ID NOs: 393 and 397 respectively; SEQ ID NOs: 401 and 405 respectively; SEQ ID NOs: 409 and 413 respectively; SEQ ID NOs: 473 and 477 respectively; SEQ ID NOs: 481 and 485 respectively; SEQ ID NOs: 489 and 493 respectively; SEQ ID NOs: 497 and 501 respectively; SEQ ID NOs: 505 and 509 respectively; SEQ ID NOs: 513 and 517 respectively; SEQ ID NOs: 545 and 549 respectively; SEQ ID NOs: 553 and 557 respectively; SEQ ID NOs: 561 and 565 respectively; SEQ ID NOs: 569 and 573 respectively; SEQ ID NOs: 577 and 581 respectively; SEQ ID NOs: 585 and 589 respectively; SEQ ID NOs: 593 and 597 respectively; SEQ ID NOs: 601 and 605 respectively; SEQ ID NOs: 625 and 629 respectively; SEQ ID NOs: 633 and 637 respectively; SEQ ID NOs: 641 and 645 respectively; SEQ ID NOs: 649 and 653 respectively; SEQ ID NOs: 657 and 661 respectively; SEQ ID NOs: 665 and 669 respectively; SEQ ID NOs: 673 and 677 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 729 and 733 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 769 and 773 respectively; SEQ ID NOs: 785 and 789 respectively; SEQ ID NOs: 793 and 797 respectively; SEQ ID NOs: 801 and 805 respectively; SEQ ID NOs: 809 and 813 respectively; SEQ ID NOs: 817 and 821 respectively; SEQ ID NOs: 849 and 853 respectively; SEQ ID NOs: 857 and 861 respectively; SEQ ID NOs: 865 and 869 respectively; SEQ ID NOs: 873 and 877 respectively; SEQ ID NOs: 881 and 885 respectively; SEQ ID NOs: 889 and 893 respectively; SEQ ID NOs: 897 and 901 respectively; SEQ ID NOs: 905 and 909 respectively; SEQ ID NOs: 913 and 917 respectively; SEQ ID NOs: 921 and 925 respectively; SEQ ID NOs: 929 and 933 respectively; SEQ ID NOs: 937 and 941 respectively; SEQ ID NOs: 945 and 949 respectively; SEQ ID NOs: 969 and 973 respectively; SEQ ID NOs: 977 and 981 respectively; SEQ ID NOs: 1009 and 1013 respectively; SEQ ID NOs: 1057 and 1061 respectively; SEQ ID NOs: 1537 and 1541 respectively; SEQ ID NOs: 1569 and 1573 respectively; SEQ ID NOs: 1601 and 1605 respectively; SEQ ID NOs: 1641 and 1645 respectively; SEQ ID NOs: 1665 and 1669 respectively; SEQ ID NOs: 1825 and 1829 respectively; SEQ ID NOs: 1865 and 1869 respectively; SEQ ID NOs: 1897 and 1901 respectively; SEQ ID NOs: 1905 and 1909 respectively; SEQ ID NOs: 1913 and 1917 respectively; SEQ ID NOs: 1921 and 1925 respectively; SEQ ID NOs: 1929 and 1933 respectively; SEQ ID NOs: 2265 and 2269 respectively; SEQ ID NOs: 2281 and 2285 respectively; 2289 and 2293 respectively; 2329 and 2333 respectively; and SEQ ID NOs: 2345 and 2349, respectively.

Additionally or alternatively, in some embodiments of the heterodimeric multispecific antibodies disclosed herein, the first immunoglobulin or the third immunoglobulin binds to a cell surface antigen selected from the group consisting of a2b b3 (Glycoprotein IIb/IIIa), a4, a4b7, a4b7+aEb7, a5, Activin receptor type-2B, ALK1, Alpha-synuclein, amyloid beta, APP, AXL, Blood Group A, CAIX, CCL-2, CD105 (endoglin), CD115 (CSF1R), CD116a (CSF2Ra), CD123, CD152 (CTLA4), CD184 (CXCR4), CD19, CD192 (CCR2), CD194 (CCR4), CD195 (CCR5), CD20, CD200, CD22, CD221 (IGF1R), CD248, CD25, CD257 (BAFF), CD26, CD262 (DR5), CD276 (B7H3), CD3, CD30 (TNFRSF8), CD319 (SLAMF7), CD33, CD332 (FGFR2), CD350 (FZD10), CD37, CD371 (CLEC12A), CD38, CD4, CD49b (a2), CD51 (a5), CD52, CD56, CD61 (a4b3), CD70, CD73 (NTSE), CD74, CEA, Claudin-18.2, cMET, CRLR, DLL3, DLL4, DNA/histone (H1) complex, EGFR, EpCAM, EGFR-HER3, EGFRvIII, EphA3, ERGT(GalNAc) Tn Antigen, FLT1, FOLR1, frizzled family receptor (FZD), Lewis Y, Lewis X, GCGR, GD2, GD2 α-acetyl, GD3, GM1, GM1 fucosyl, GM2, GPA33, GPNMB, GUCY2C, HER2, HER3, HGFR (cMET), IgHe, IGLF2, Kallikreins, LINGO1, LOXL2, Ly6/PLAUR domain-containing protein 3, MADCAM1, MAG, Mesothelin, MT1-MMP (MMP14), MUC1, Mucin SAC, NaPi2b, NeuGc-GM3, notch, NOTCH2/NOTCH3 receptors, oxLDL, P-selectin, PCSK9, PDGFRA, PDGFRa, phosphatidylserine, polysialic acid, PSMA, PVRL4, RGMA, CD240D Blood group D antigen, root plate-specific spondin 3, serum amyloid P component, STEAP-1, TACSTD2, TGFb, TWEAKR, TYRP1, VEGFR2, VSIR, CD171 (L1CAM), CD19, CD47, pMHC[NY-ESO1], pMHC[MART1], pMHC[MAGEA1], pMHC[Tyrosinase], pMHC[gp100], pMHC[MUC1], pMHC[tax], pMHC[WT-1], pMHC[EBNA-1], pMHC[LMP2], pMHC[hTERT], GPC3, CD80, CD23, and fibronectin extra domain-B. The first immunoglobulin and the third immunoglobulin may bind to the same epitope on a target cell or two different epitopes on a target cell. In some embodiments, the target cell is a cancer cell.

Additionally or alternatively, in some embodiments of the heterodimeric multispecific antibodies disclosed herein, the second immunoglobulin or the fourth immunoglobulin bind to an epitope on a white blood cell, a monocyte, a lymphocyte, a granulocyte, a macrophage, a T cell, a NK cell, a B cell, a NKT cell, an ILC, or neutrophil.

In any of the above embodiments of the heterodimeric multispecific antibodies disclosed herein, the second immunoglobulin or the fourth immunoglobulin bind to an antigen selected from the group consisting of Dabigatran, a4, a4b7, a4b7+aEb7, a5, AXL, BnDOTA, CD11a (LFA-1), CD3, CD4, CD8, CD16, CD19, CD22, CD23, CD25, CD28, CD30 (TNFRSF8), CD33, CD38, CD40, CD40L, CD47, CD49b (a2), CD54 (ICAM-1), CD56, CD74, CD80, CD115 (CSF1R), CD116a (CSF2Ra), CD123, CD134 (OX40), CD137 (41BB), CD152 (CTLA4), CD184 (CXCR4), CD192 (CCR2), CD194 (CCR4), CD195 (CCR5), CD223 (LAG-3), CD252 (OX40L), CD254 (RANKL), CD262 (DR5), CD27, CD200, CD221 (IGF1R), CD248, CD274 (PD-L1), CD275 (ICOS-L), CD278 (ICOS), CD279 (PD-1), CD319 (SLAMF7), CD371 (CLEC12A), MADCAM1, MT1-MMP (MMP14), NKG2A, NRP1,TIGIT, VSIR, KIRDL1/2/3, and KIR2DL2. The second immunoglobulin and the fourth immunoglobulin may bind to the same epitope or different epitopes on a white blood cell, a monocyte, a lymphocyte, a granulocyte, a macrophage, a T cell, a NK cell, a B cell, a NKT cell, an ILC, or neutrophil. In some embodiments, the second immunoglobulin binds CD3 and the fourth immunoglobulin binds an immune cell receptor selected from the group consisting of CD4, CD8, CD25, CD28, CTLA4, OX40, ICOS, PD-1, PD-L1, 41BB, CD2, CD69, and CD45. In other embodiments, the second immunoglobulin binds CD16 and the fourth immunoglobulin binds an immune cell receptor selected from the group consisting of CD56, NKG2D, and KIRDL1/2/3. In certain embodiments, the fourth immunoglobulin binds to an agent selected from the group consisting of a cytokine, a nucleic acid, a hapten, a small molecule, a radionuclide, an immunotoxin, a vitamin, a peptide, a lipid, a carbohydrate, biotin, digoxin, or any conjugated variants thereof.

Additionally or alternatively, in some embodiments of the heterodimeric multispecific antibodies disclosed herein, the first immunoglobulin and the third immunoglobulin bind to their respective epitopes with a monovalent affinity or an effective affinity between about 100 nM to about 100 pM. In certain embodiments, the first immunoglobulin and the third immunoglobulin bind to cell surface epitopes that are between 60 and 120 angstroms apart.

Additionally or alternatively, in some embodiments of the heterodimeric multispecific antibodies disclosed herein, the first immunoglobulin and the third immunoglobulin bind to their respective epitopes with a monovalent affinity or an effective affinity that is less than 100 pM. In certain embodiments, the first immunoglobulin and the third immunoglobulin bind to cell surface epitopes that are up to 180 angstroms apart.

Additionally or alternatively, in some embodiments of the heterodimeric multispecific antibodies disclosed herein, the first heterodimerization domain of the first immunoglobulin and/or the second heterodimerization domain of the third immunoglobulin is a CH2-CH3 domain and has an isotype selected from the group consisting of IgG1, IgG2, IgG3, IgG4, IgA1, IgA2, IgM, IgD, and IgE.

Additionally or alternatively, in some embodiments of the heterodimeric multispecific antibodies disclosed herein, the first heterodimerization domain of the first immunoglobulin and/or the second heterodimerization domain of the third immunoglobulin comprises an IgG1 constant region comprising one or more amino acid substitutions selected from the group consisting of N297A and K322A. Additionally or alternatively, in some embodiments of the heterodimeric multispecific antibodies disclosed herein, the first heterodimerization domain of the first immunoglobulin is a CH2-CH3 domain comprising a K409R mutation and the second heterodimerization domain of the third immunoglobulin is a CH2-CH3 domain comprising a F405L mutation.

Also disclosed herein are recombinant nucleic acid sequences encoding any of the antibodies described herein. In another aspect, the present technology provides a host cell or vector expressing any nucleic acid sequence encoding any of the antibodies described herein.

In any of the above embodiments of the immunoglobulin-related compositions of the present technology, the HDTVS antibody may be optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof.

In one aspect, the present disclosure provides a method for treating cancer in a subject in need thereof, comprising administering to the subject an effective amount of a heterodimeric multispecific antibody disclosed herein. The cancer may be lung cancer, colorectal cancer, skin cancer, breast cancer, ovarian cancer, leukemia, pancreatic cancer, or gastric cancer. Additionally or alternatively, in some embodiments, the heterodimeric multispecific antibody is administered to the subject separately, sequentially or simultaneously with an additional therapeutic agent.

Also disclosed herein are kits for detection and/or treatment of a disease (e.g., cancers), comprising at least one heterodimeric trivalent/tetravalent multispecific antibody of the present technology and instructions for use.

### BRIEF DESCRIPTION OF THE DRAWINGS

FIG. 1a shows the basic design strategy of each HeteroDimeric TetraValency and Specificity (HDTVS) variant compared with the parental 2+2 IgG-[L]-scFv. The 5 heterodimeric IgG-L-scFv designs display novel biological activities. Each construct uses heterodimerization to achieve tri- or tetraspecificity.

FIG. 1b shows a schematic of the 1+1+2 Low affinity design and how it can be used to distinguish single-antigen positive healthy cells from dual-antigen positive target cells. Single antigen positivity would result in inferior immune cell activation over dual antigen positivity.

FIG. 1c shows a schematic of the 1+1+2 High affinity design and how it can be used to target either (or both) of two different cellular antigens.

FIG. 1d shows a schematic of the 2+1+1 design and how it can be used to improve immune cell activation. Targeting of two different immune cell receptors can be used to more specifically recruit an immune cell population or provide greater immune cell activation or inhibition through cross linking of multiple receptors.

FIG. 1e shows a schematic of the 2+1+1 design and how it can be used to broaden immune cell recruitment or combine payload delivery with immunotherapy. Each HDTVS antibody needs only one immune cell receptor for recruitment and activation. The additional domain can then be used to bind payloads (for diagnostics, therapy, recruitment, etc.) or additional effector cells.

FIG. 1f shows a schematic of the 1+1+1+1 design and how it can be used to combine the benefits of 1+1+2 with 2+1+1. In this embodiment, tetraspecificity can bring better specificity or a broader range of targets, as well and improved immune cell activation or payload delivery.

FIG. 2a shows the superior cytotoxicity, binding and in vivo potency of the IgG-[L]-scFv design over the IgG-Het and BiTE formats. A 4 hr Cr⁵¹′ release assay was used to evaluate cytotoxicity of activated T-cells against M14 melanoma tumor cells. Flow cytometry was used to evaluate differences in antigen binding of each bispecific antibody to huCD3 or GD2 on activated T cells or M14 melanoma tumor cells, respectively. Affinities were measured using SPR on GD2 coated streptavidin chips. Two mouse models were used for assessing in vivo potency, a syngeneic transgenic model which has huCD3 expressing murine T cells, and a humanized xenograft model using activated human T-cells engrafted into immunodeficient IL2-re^{-/-} Rag2^{-/-} BALB/c mice. Mice were implanted subcutaneously with GD2(+) tumors and treated intravenously with a particular test bispecific antibody.

FIG. 2b shows the superior cytotoxicity of the IgG-[L]-scFv design over the IgG-het using two additional anti-GD2 sequences.

FIG. 3 shows the schematics of 4 IgG-[L]-scFv heterodimeric variants along with the parental format and the IgG-Het format. Designs are ranked by their relative potency.

FIG. 4 shows the in vitro binding activity of the various IgG-[L]-scFv variants. GD2 and CD3 affinities were measured using SPR with GD2 or huCD3de coated chips, respectively. Cell binding was assayed by flow cytometry using activated human T cells or M14 melanoma cells. T-cell: tumor cell conjugate formation was measured by flow cytometry using differentially labeled activated human T cells and M14 melanoma tumor cells.

FIG. 5 shows the in vitro cytotoxicity of each IgG-[L]-scFv variant against two cell lines: M14 melanoma and IMR32 neuroblastoma. Cytotoxicity was measured using a 4 hr Cr⁵¹ release assay and activated human T-cells.

FIG. 6 shows the in vitro immune cell activation of each IgG-[L]-scFv variant. Activation was measured by flow cytometry. Naïve purified T cells and M14 melanoma cells were co-cultured for 24 or 96 hrs, harvested and stained for CD69 or CD25, respectively. T cells for the 96 hr time points were also labeled with Cell Trace Violet (CTV). Culture supernatant was also collected at the 24 hr time point for cytokine measurements.

FIG. 7 shows the in vivo activity of each IgG-[L]-scFv variant. Two mouse models were used for assessing in vivo potency, a syngeneic transgenic model which has huCD3 expressing murine T cells, and a humanized xenograft model using activated human T-cells engrafted into immunodeficient IL2-rg^{-/-} Rag2^{-/-} BALB/c mice. Mice were implanted subcutaneously with GD2(+) tumors and treated intravenously with a particular test bispecific antibody.

FIG. 8 shows various dual bivalent bispecific antibody formats compared to the IgG-[L]-scFv design. Cytotoxicity was evaluated using a 4 hr Cr⁵¹ release assay using activated human T cells and M14 melanoma cells. Conjugation activity was measured using flow cytometry. Cell binding was evaluated by flow cytometry using activated human T cells and M14 melanoma cells.

FIG. 9 shows IgG-[L]-scFv variants which bind CD33 or HER2. Cell binding activities were measured by flow cytometry using Molm13, SKMEL28, or MCF7 cells. Cytotoxicity was assessed using Molm13 cells and activated human T cells in a 4 hr Cr⁵¹ release assay.

FIG. 10a shows two 1+1+2 designs (high and low affinity variants). Cell binding and cytotoxicity assays used the GD2(+)HER2(+) cell line U2OS. Cytotoxicity was measured using 4 hr Cr⁵¹ release, and cell binding was evaluated using flow cytometry.

FIG. 10b shows two 1+1+2 designs (high and low affinity variants). Cell binding and cytotoxicity assays used the GD2(+) IMR32 neuroblastoma cells or HER2(+) HCC1954 breast cancer cells. Cytotoxicity was measured using 4 hr Cr⁵¹ release, and cell binding was evaluated using flow cytometry.

FIGS. 11a-11e show exemplary Fc variants that are capable of heterodimerization.

FIG. 12a shows various dual bivalent bispecific antibody formats compared in vivo to the IgG-[L]-scFv design. Schematics show all four dual bivalent bispecific antibodies expressed.

FIG. 12b shows the mean tumor growth for in vivo huDKO arming model. Tumor responses were evaluated using a T-cell arming model, where T-cells were preincubated with each BsAb for 20 min at a concentration to achieve equal anti-GD2 binding domains (as verified by flow cytometry). These prelabeled or “armed” T-cells were injected intravenously into tumor bearing DKO mice. Each line represents one BsAb. Solid black triangles represent a dose of BsAb armed human activated T-cells (huATC) and IL-2. The dotted black line represents no measurable tumor and the star represents the tumor implantation. Error bars represent standard deviation.

FIG. 12c shows tumor growth from individual mice. Each figure represents one treatment group, with schematics (see above) for reference. Each solid line represents a single mouse, and the dotted lines represents the group average.

FIG. 13 demonstrates the combined binding effect of L1CAM/GD2 1+1+2 Lo, a heterodimeric 1+1+2Lo format antibody that can bind ganglioside GD2 and adhesion protein L1CAM simultaneously. Design of the 1+1+2 Lo format antibody is shown on the left side. Homodimeric formats against GD2 and L1CAM were included for reference. For this binding assay, Neuroblastoma cells (IMR32) were incubated with each antibody for 30 minutes at 4° C., washed and incubated with a fluorescent anti-human secondary antibody. After the final wash, the cells were analyzed using flow cytometry. In this example, the binding of the low affinity 1+1+2 HDTVS antibody was stronger than the anti-L1CAM homodimeric antibody, but weaker than the anti-GD2 homodimeric antibody, thus showing improved targeting specificity for tumors expressing both GD2 and L1CAM.

FIG. 14 demonstrates the combined binding effect of HER2/EGFR 1+1+2 Hi, a heterodimeric 1+1+2Hi format antibody that can bind both HER2 and EGFR, either simultaneously or separately. Design of the 1+1+2 Hi format antibody is shown on the right side. Homodimeric formats against HER2 and EGFR were included for reference. For this binding assay, Desmoplastic Small Cell Round Tumor cells (JN-DSRCT1) were incubated with each antibody for 30 minutes at 4° C., washed and incubated with a fluorescent anti-human secondary antibody. After the final wash, the cells were analyzed using flow cytometry. In this example, the binding of the high affinity 1+1+2 HDTVS antibody was stronger than that of either anti-HER2 or anti-EGFR homodimeric antibodies, while maintaining specificity for both antigens, demonstrating cooperative binding.

FIG. 15 demonstrates the combined binding effect of GD2/B7H3 1+1+2 Lo, a heterodimeric 1+1+2Lo format antibody that can bind both GD2 and B7H3 simultaneously. Design of the 1+1+2 Lo format antibody is shown on the left hand side. Homodimeric formats against GD2 and B7H3, and monovalent control antibodies against GD2 or B7H3 (GD2 or B7H3 ctrl, respectively) were included for reference. For this binding assay, Osteosarcoma cells (U2OS) were incubated with each antibody for 30 minutes at 4° C., washed and incubated with a fluorescent anti-human secondary antibody. After the final wash, the cells were analyzed using flow cytometry. In this example, the binding of the low affinity 1+1+2 HDTVS antibody was similar to the anti-B7H3 homodimeric antibody, but weaker than the anti-GD2 homodimeric antibody. Importantly, GD2/B7H3 1+1+2 Lo also showed improved binding over monovalent control antibodies, demonstrating cooperative binding.

FIG. 16 demonstrates the cytotoxic selectivity of HER2/GD2 1+1+2 Lo, a heterodimeric 1+1+2Lo format that can bind both GD2 and HER2 simultaneously. In this format, a low affinity HER2 sequence was used. Design of the 1+1+2 Lo format antibody is shown below the line graph. Homodimeric formats against GD2 and HER2, and monovalent control antibodies against GD2 or HER2 (GD2 and HER2 ctrl, respectively) were included for reference. For this cytotoxicity assay, Osteosarcoma cells (U2OS) were first incubated with ⁵¹Cr for one hour. After the incubation, the ⁵¹Cr labeled target cells were mixed with serial dilutions of the indicated antibody and activated human T-cells for four hours at 37° C. After four hours, supernatant was harvested and analyzed on a gamma counter to quantify the released ⁵¹Cr. Cytotoxicity was measured as the % of released ⁵¹Cr from maximum release. In this example, the low affinity 1+1+2 heterodimer antibody killed the target cells as effectively as the anti-GD2 and anti-HER2 homodimeric antibodies yet showing clear superiority over the monovalent control formats. This demonstrates the selectivity possible with the 1+1+2Lo design: target cells expressing each individual antigen will be targeted with 10-100-fold lower cytotoxic potency than targets expressing both antigens simultaneously. Using a homodimeric design for either GD2 or HER2 would lose such selectivity.

FIG. 17a demonstrates the cytotoxic dual specificity of HER2/GPA33 1+1+2 Hi, a heterodimeric 1+1+2Hi format that can bind both GPA33 and HER2 simultaneously. Design of the 1+1+2 Hi format antibody is shown below the line graph. Homodimeric formats against GPA33 and HER2, and monovalent control antibodies against GPA33 or HER2 were included for reference. For this cytotoxicity assay, Colon cancer cells (Colo205) were first incubated with ⁵¹Cr for one hour. After the incubation, the ⁵¹Cr labeled target cells were mixed with serial dilutions of the indicated antibody and activated human T-cells for four hours at 37° C. After four hours, the supernatant was harvested and read on a gamma counter to quantify the released ⁵¹Cr. Cytotoxicity was measured as the % of released ⁵¹Cr from maximum release. In this example, the high affinity 1+1+2 heterodimer antibody killed target cells as effectively as the anti-GPA33 homodimeric antibody, but with greater potency than the anti-HER2 homodimeric antibody and monovalent control antibodies. These data demonstrate functional cooperativity between the HER2 and GPA33 antigen-binding domains and illustrate that the dual specificity of a 1+1+2Hi format does not significantly compromise its cytotoxicity against either antigen individually.

FIG. 17b demonstrates the combined binding effect of HER2/GPA33 1+1+2 Hi, a heterodimeric 1+1+2Hi format that can bind both HER2 and GPA33, either simultaneously or separately. Design of the 1+1+2 Hi format antibody is shown on the right hand side. For this binding assay, Colon cancer cells (Colo205) were incubated with each antibody for 30 minutes at 4° C., washed and incubated with a fluorescent anti-human secondary antibody. After the final wash, the cells were analyzed using flow cytometry. In this example, the affinity binding of the 1+1+2 heterodimer antibody was stronger than either anti-HER2 or anti-GPA33 homodimeric and monovalent control antibodies, while maintaining specificity for both antigens, demonstrating cooperative binding.

FIG. 18 demonstrates the utility of CD3/CD28 2+1+1, a heterodimeric 2+1+1 design that can bind both CD3 and CD28 on T-cells. Design of the heterodimeric 1+1+2 format antibody is shown below the line graph. Homodimeric formats against CD3 and CD28 were included for reference. For this cytokine assay, naïve human T-cells and Melanoma tumor cells (M14) were co-cultured along with the indicated BsAb for 20 hours before culture supernatants were harvested and analyzed for secreted cytokine IL-2 by flow cytometry. Data was normalized to T-cell cytokine release after 20 hours without target cells or antibody. The CD3/CD28 2+1+1 design showed clearly more potent cytokine release activity than either CD3 or CD28 engagement alone, illustrating cooperative activity from dual CD3/CD28 engagement.

FIG. 19 demonstrates the combined binding effect of CD3/CD4 2+1+1, a heterodimeric 2+1+1 format antibody that can bind both CD3 and CD4 simultaneously. Design of the heterodimeric 2+1+1 format antibody is shown on the right side. For this binding assay, active human T cells were incubated with each antibody for 30 minutes at 4° C., washed and incubated with a fluorescent anti-human secondary antibody. After the final wash, the cells were analyzed using flow cytometry. In this example, the 2+1+1 heterodimer shows enhanced binding compared to the bivalent CD4 and monomeric CD3 binder (2+1) demonstrating cooperative binding.

FIG. 20 demonstrates the combined binding effect of CD3/PD-1 2+1+1, a heterodimeric 2+1+1 format antibody that can bind both CD3 and PD-1 simultaneously. Design of the heterodimeric 2+1+1 format antibody is shown on the right side. For this binding assay active human T cells were incubated with each antibody for 30 minutes at 4° C., washed and incubated with a fluorescent anti-human secondary antibody. After the final wash, the cells were analyzed using flow cytometry. In this example, the binding of the 2+1+1 heterodimer was better than either anti-PD-1 homodimeric or anti-CD3 monomeric (2+1) binder, demonstrating cooperative binding.

FIGS. 21a-21c show the unique characteristics of the IgG-L-scFv design, compared to two other common dual bivalent design strategies: the BiTE-Fc and the IgG-H-scFv. FIG. 21a demonstrates the potent T-cell functional activity of the IgG-L-scFv design compared to other dual bivalent T-cell bispecific antibody formats. Designs of the IgG-L-scFv, BiTE-Fc and the IgG-H-scFv format antibodies are shown below the line graph. For this cytokine assay, naïve T-cells and melanoma tumor cells (M14) were co-cultured along with each BsAb for 20 hours before culture supernatants were harvested and analyzed for secreted cytokine IL-2 by flow cytometry. Data were normalized to T-cell cytokine release after 20 hours without target cells or antibody. In contrast to the IgG-H-scFv (2+2HC) and the BiTE-Fc (2+2B) designs, the IgG-L-scFv format (2+2) demonstrated significant cytokine IL-2 responses in vitro, which correlated with stronger in vivo activity (shown in FIG. 21c). FIG. 21b illustrates the unusually weak T-cell binding activity of the IgG-L-scFv design compared to other dual bivalent T-cell bispecific antibody formats. For this binding assay, T-cells and melanoma tumor cells (M14) were separately incubated with each antibody for 30 minutes at 4° C., washed and incubated with a fluorescent anti-human secondary antibody. After the final wash, the cells were analyzed using flow cytometry. Shown is CD3-specific (FIG. 21b, upper panel), and GD2-specific binding (FIG. 21b, middle panel). Designs of the IgG-L-scFv, BiTE-Fc and the IgG-H-scFv format antibodies are shown in FIG. 21b (lower panel). In contrast to their GD2 binding activity, each BsAb demonstrated quite different T-cell binding activities. These data demonstrated how the IgG-L-scFv design is uniquely different than other dual-bivalent designs, with each scFv showing incomplete bivalent binding. Although the inclusion of two scFv domains in the IgG-L-scFv does show improvement over monovalent designs, it still does not compare to the binding activity of the 2+2HC or 2+2B designs, illustrating the sterically hindered binding of this format. FIG. 21c illustrates the in vivo superiority of the IgG-L-scFv design. In contrast to other dual bivalent designs, the IgG-L-scFv format was the only one capable of controlling tumor growth in mice. Here, immunodeficient mice (Balb/c IL-2Rgc−/−, Rag2−/−) were implanted with neuroblastoma cells (IMR32) subcutaneously, before being treated with intravenous activated T-cells and antibody (2-times per week). Tumors sizes were measured by caliper.

FIG. 22 demonstrates the in vitro properties and design of anti-CD33/CD3 IgG-[L]-scFv panel. The in vitro cytotoxicity EC₅₀, fold-difference in EC₅₀, antigen valency, heterodimer design and protein purity by SEC-HPLC for anti-CD33/CD3 IgG-[L]-scFv panel are summarized. Fold change is based on the EC₅₀ of 2+2. Purity was calculated as the fraction of protein at correct elution time out of the total protein by area under the curve of the SEC-HPLC chromatogram. For the cytotoxicity assays, CD33-transfected cells (Nalm6) were first incubated with ⁵¹Cr for one hour. Afterwards, ⁵¹Cr labeled target cells were mixed with serial titrations of the indicated antibody and activated human T-cells for four hours at 37° C. The supernatant was harvested and analyzed on a gamma counter to quantify the released ⁵¹Cr. Cytotoxicity was measured as the % of released ⁵¹Cr from maximum release. These results confirm the relative importance of Cis-oriented binding domains in an additional antigen system (CD33) which is much more membrane distal than GD2 (see FIG. 5).

FIG. 23 provides a summary of the various HDTVS antibodies tested in the Examples disclosed herein. The table summarizes all successfully produced HDTVS formatted multi-specific antibodies across a variety of antigen models. All clones were expressed in Expi293 cells and heterodimerized using the controlled Fab Arm Exchange method. HDTVS type displays the category of each clone. Fab 1 and scFv 1 (and corresponding Ag1 and Ag3) are attached in a cis-orientation on one heavy chain (linked by the light chain of Fab) while Fab 2 and scFv 2 (and corresponding Ag2 and Ag4) are on a separate heavy chain molecule in a cis-orientation (linked by the light chain of Fab).

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Advances in protein engineering can enhance the functional output of proteins by linking different peptides in sequences, or by arranging them in complexes that do not exist naturally. Antibodies have served as a platform for such enhancements, where antigen binding can be modulated through antigen affinity maturation (Boder et al., Proc Natl Acad Sci USA 97:10701-10705 (2000)) or increases in valency (Cuesta et al., Trends Biotechnol 28:355-362 (2010)). Fc receptor binding can be modulated through point mutations (Leabman et al., MAbs 5:896-903 (2013)) or changes in glycosylation (Xu et al., Cancer Immun Res 4: 631-638 (2016)) whereas pharmacokinetics can be influenced through ablation of FcR(n) binding (Suzuki et al., J Immunol 184:1968-1976 (2010)) or removal of entire antibody domains. However, no single antibody platform to date has shown a clear and significant functional advantage over others within the clinic.

The present disclosure provides an antibody platform in which up to 4 different antigen binding domains can be used to simultaneously engage up to 4 different cellular targets, thereby increasing avidity and modulating specificity of the therapeutic antibodies. This platform is based on the heterodimerization of two IgG half molecules, in which each IgG half molecule comprises a heavy chain and a light chain, wherein a scFv is linked to the C-terminus of at least one light chain (i.e., IgG-[L]-scFv platform). The resulting heterodimers are both trivalent/tetravalent and multispecific and are collectively referred to as HDTVS antibodies.

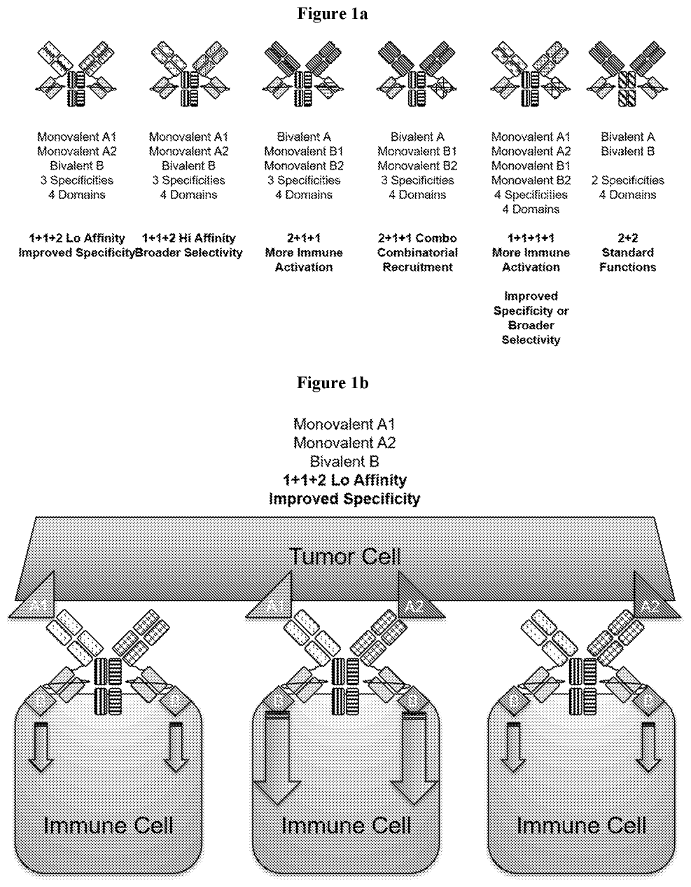

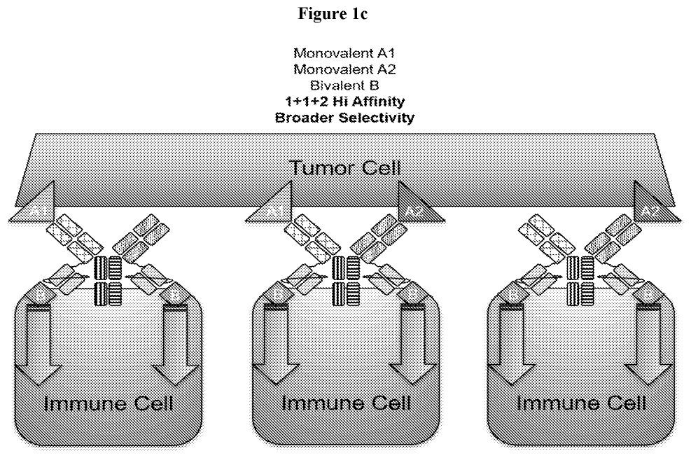

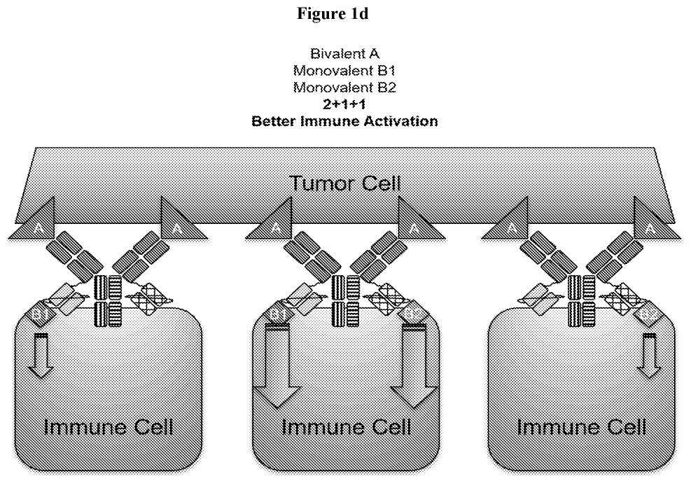

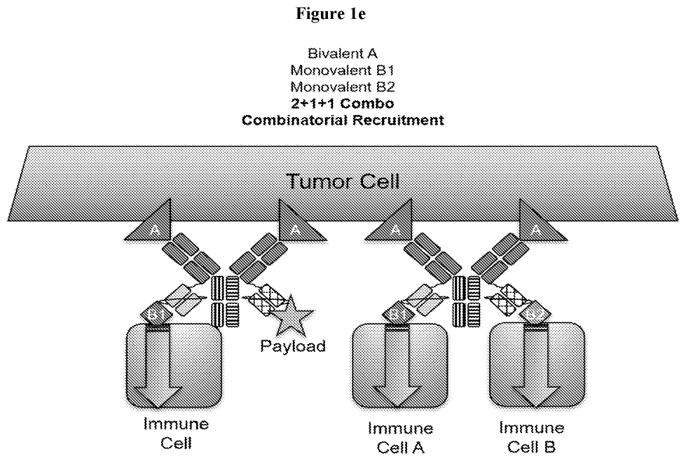

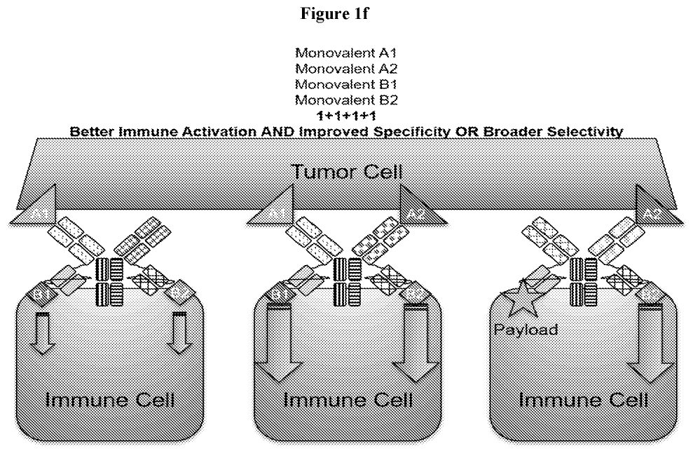

The native form of the IgG-[L]-scFv platform has bivalent binding to two different targets (2+2) (each integer represents a different specificity, while its value represents the valency). The present disclosure provides 5 HDTVS platform variants which vary the 4 functional domains (2 Fabs and 2 scFv) in the IgG(L)-scFv format: (1) the Lo1+1+2 HDTVS variant to achieve improved tumor cell specificity, (2) the Hi1+1+2 HDTVS variant to achieve broader tumor cell selectivity, (3) the 2+1+1 HDTVS variant to achieve improved immune cell activation, (4) the 2+1+1 HDTVS variant which allows recruitment of different cells and/or payloads and (5) the 1+1+1+1 HDTVS variant which combines designs from (1) or (2) with (3) or (4) to achieve more effective immune activation or payload delivery with finer specificity or broader selectivity. (FIGS. 1a-1f). In order to test the functional output of these HDTVS variants, one of the 2 Fab domains can be neutralized by using an irrelevant Fab that has no binding to either tumor cells or effector immune cells (e.g., T cells), creating monovalency for tumor. Alternatively, one of the scFv domains can be removed to create monovalency towards effector immune cells (e.g., T cells).

As described herein, the biological potency of each design is dependent on the biophysical characteristics of the antigen binding domains of the HDTVS variants. Unexpectedly, the changes in valency did not entirely correlate with changes in functional output. As shown in Examples described herein, the biological activity of the tri- and tetra-specific variants of the HDTVS platform is dependent on the antigen/epitope combinations, as well as the relative binding affinities to each target antigen (up to 4 targets total). The Lo1+1+2 HDTVS variant requires its Fab domains to bind to two distinct tumor antigens that are within a proximity of 60-120 angstroms from each other (thus allowing simultaneous binding), and (b) have monovalent and/or effective binding affinities (K_{D}) that range from about 100 nM to about 100 pM to reduce bystander reactivity with healthy cells. The Hi1+1+2 HDTVS variant on the other hand exploits the high monovalent and/or effective binding affinity (K_{D}<100 pM) of its Fab domains such that monovalency is nearly as effective as bivalency. Moreover, the 2+1+1 HDTVS variant exhibited in vivo tumor clearance activity that was comparable to that observed with the 2+2 native form of the IgG-[L]-scFv platform. These results were unexpected given that the binding activities of the 2+1+1 HDTVS variant were about 6-fold lower than the 2+2 native form of the IgG-[L]-scFv platform.

Accordingly, biophysical properties such as orientation (cis vs trans), valency (mono- vs bi-valent) and target affinity (K_{D}˜nM or <pM) had an unpredictable impact on the functionality of the HDTVS variants (e.g., log-fold enhancement of therapeutic efficacy). Moreover, the HDTVS antibodies of the present technology show superior therapeutic potency compared to other conventional antibody platforms, such as BiTE or heterodimeric IgG (IgG-Het). These results also demonstrate that different multispecific antibody platforms yield antibodies that possess substantially different biological properties. Without wishing to be bound by theory, it is believed that spatial distances between the antigen binding domains of multispecific antibodies, as well as the relative flexibility and orientation of the individual antigen binding domains may determine their ability to drive cell-to-cell interactions.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, a “2+1+1” design refers to a HDTVS antibody in which the two Fab domains recognize and bind to the same target antigen, and the two scFvs recognize and bind to two distinct target antigens. In some embodiments, the two scFvs of the 2+1+1 HDTVS antibody binds to two distinct target antigens that are up to 180 angstroms apart from each other in order to engage two separate molecules on the same cell.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ greater, at least 10⁴M⁻¹ greater or at least 10⁵ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the VH region and the VL region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds a target antigen will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). “Immunoglobulin-related compositions” as used herein, refers to antibodies (including monoclonal antibodies, polyclonal antibodies, humanized antibodies, chimeric antibodies, recombinant antibodies, multispecific antibodies, bispecific antibodies, etc.,) as well as antibody fragments. An antibody or antigen binding fragment thereof specifically binds to an antigen.

As used herein, the term “antibody-related polypeptide” means antigen binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CH₁, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full-length antibody, generally the antigen binding or variable region thereof. Examples of antibody fragments or antigen binding fragments include Fab, Fab′, F(ab′)₂, and Fv fragments; diabodies; linear antibodies; single-chain antibody molecules; and multispecific antibodies formed from antibody fragments.

“Bispecific antibody” or “BsAb”, as used herein, refers to an antibody that can bind simultaneously to two targets that have a distinct structure, e.g., two different target antigens, two different epitopes on the same target antigen, or a hapten and a target antigen or epitope on a target antigen. A variety of different bispecific antibody structures are known in the art. In some embodiments, each antigen binding moiety in a bispecific antibody includes V_{H} and/or V_{L} regions; in some such embodiments, the V_{H} and/or V_{L} regions are those found in a particular monoclonal antibody. In some embodiments, the bispecific antibody contains two antigen binding moieties, each including VH and/or VL regions from different monoclonal antibodies. In some embodiments, the bispecific antibody contains two antigen binding moieties, wherein one of the two antigen binding moieties includes an immunoglobulin molecule having VH and/or VL regions that contain CDRs from a first monoclonal antibody, and the other antigen binding moiety includes an antibody fragment (e.g., Fab, F(ab′), F(ab′)₂, Fd, Fv, dAB, scFv, etc.) having VH and/or VL regions that contain CDRs from a second monoclonal antibody.

As used herein, the term “diabodies” refers to small antibody fragments with two antigen binding sites, which fragments comprise a heavy-chain variable domain (VH) connected to a light-chain variable domain (VL) in the same polypeptide chain (VH VL). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and 30 Hollinger et al., Proc. Natl. Acad. Sci. USA, 90: 6444-6448 (1993).

As used herein, the terms “single-chain antibodies” or “single-chain Fv (scFv)” refer to an antibody fusion molecule of the two domains of the Fv fragment, VL and VH. Single-chain antibody molecules may comprise a polymer with a number of individual molecules, for example, dimer, trimer or other polymers. Furthermore, although the two domains of the Fᵥ fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fᵥ (scFv)). Bird et al. (1988) Science 242:423-426 and Huston et al. (1988) Proc. Natl. Acad Sci. USA 85:5879-5883. Such single-chain antibodies can be prepared by recombinant techniques or enzymatic or chemical cleavage of intact antibodies.

Any of the above-noted antibody fragments are obtained using conventional techniques known to those of skill in the art, and the fragments are screened for binding specificity and neutralization activity in the same manner as are intact antibodies.

As used herein, an “antigen” refers to a molecule to which an antibody (or antigen binding fragment thereof) can selectively bind. The target antigen may be a protein, carbohydrate, nucleic acid, lipid, hapten, or other naturally occurring or synthetic compound. In some embodiments, the target antigen may be a polypeptide. An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFvFc, Fab, Fab′ and F(ab′)₂, but are not limited thereto.

By “binding affinity” is meant the strength of the total noncovalent interactions between a single binding site of a molecule (e.g., an antibody) and its binding partner (e.g., an antigen). The affinity of a molecule X for its partner Y can generally be represented by the dissociation constant (Ku). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a breast, lung, colon, or prostate tissue sample obtained by needle biopsy.

As used herein, the term “cancer” refers to a neoplasm or tumor resulting from abnormal uncontrolled growth of cells. In some embodiments, cancer refers to a benign tumor or a malignant tumor. In some embodiments, the cancer is associated with a specific cancer antigen.

As used herein, the term “CDR-grafted antibody” means an antibody in which at least one CDR of an “acceptor” antibody is replaced by a CDR “graft” from a “donor” antibody possessing a desirable antigen specificity.

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See generally, Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 0125,023; Better et al., Science 240: 1041-1043, 1988; Liu et al., Proc. Natl. Acad. Sci. USA 84: 3439-3443, 1987; Liu et al., J. Immunol 139: 3521-3526, 1987; Sun et al., Proc. Natl. Acad. Sci. USA 84: 214-218, 1987; Nishimura et al., Cancer Res 47: 999-1005, 1987; Wood et al., Nature 314: 446-449, 1885; and Shaw et al., J. Natl. Cancer Inst. 80: 1553-1559, 1988.

As used herein, the term “consensus FR” means a framework (FR) antibody region in a consensus immunoglobulin sequence. The FR regions of an antibody do not contact the antigen.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective affinity” refers to the binding constant derived from measuring the overall binding kinetics of a compound with two or more simultaneous binding interactions (e.g., with an IgG, IgM, IgA, IgD, or IgE molecule instead of a Fab domain).

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “effector cell” means an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Effector cells express specific Fc receptors and carry out specific immune functions. An effector cell can induce antibody-dependent cell-mediated cytotoxicity (ADCC), e.g., a neutrophil capable of inducing ADCC. For example, monocytes, macrophages, neutrophils, eosinophils, and lymphocytes which express FcaR are involved in specific killing of target cells and presenting antigens to other components of the immune system, or binding to cells that present antigens.

“Effector function” as used herein refers to a biochemical event that results from the interaction of an antibody Fc region with an Fc receptor or an antigen. Effector functions include but are not limited to antibody dependent cell mediated cytotoxicity (ADCC), antibody dependent cell mediated phagocytosis (ADCP), and complement dependent cytotoxicity (CDC). Effector functions include both those that operate after the binding of an antigen and those that operate independent of antigen binding.

As used herein, the term “epitope” means an antigenic determinant (site on an antigen) capable of specific binding to an antibody. Epitopes usually comprise chemically active surface groupings of molecules such as amino acids or sugar side chains and may have specific three-dimensional structural characteristics, as well as specific charge characteristics. Conformational and non-conformational epitopes are distinguished in that the binding to the former but not the latter is lost in the presence of denaturing solvents. Thus, in some embodiments, the heterodimeric trivalent/tetravalent multispecific antibodies disclosed herein may bind a non-conformational epitope and/or a conformational epitope. To screen for antibodies which bind to an epitope, a routine cross-blocking assay such as that described in Antibodies, A Laboratory Manual, Cold Spring Harbor Laboratory, Ed Harlow and David Lane (1988), can be performed. This assay can be used to determine if an antibody binds the same site or epitope as a heterodimeric trivalent/tetravalent multispecific antibody of the present technology. Alternatively, or additionally, epitope mapping can be performed by methods known in the art. For example, the antibody sequence can be mutagenized such as by alanine scanning, to identify contact residues. In a different method, peptides corresponding to different regions of a target protein antigen can be used in competition assays with the test antibodies or with a test antibody and an antibody with a characterized or known epitope.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, a “heterodimerization domain that is incapable of forming a stable homodimer” refers to a member of a pair of distinct but complementary chemical motifs (e.g., amino acids, nucleotides, sugars, lipids, synthetic chemical structures, or any combination thereof) which either exclusively self-assembles as a heterodimer with the second complementary member of the pair, or shows at least a 10⁴ fold preference for assembling into a heterodimer with the second complementary member of the pair, or forms a homodimer with an identical member that is not stable under reducing conditions such as >2 mM 2-MEA at room temperature for 90 minutes (see e.g., Labrijn, A. F. et al., Proc. Natl. Acad. Sci. 110, 5145-50 (2013). Examples of such heterodimerization domains include, but are not limited to CH2-CH3 that include any of the Fc variants/mutations described herein, WinZip-A1B1, a pair of complementary oligonucleotides, and a CH-1 and CL pair.

As used herein, “Hi1+1+2” refers to a heterodimeric tetravalent multispecific antibody in which the Fab domains (a) bind to two distinct target epitopes and (b) have monovalent binding affinities or effective affinities (K_{D}) that are <100 pM.

As used herein, the term “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR is typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992). See e.g., Ahmed & Cheung, FEBS Letters 588(2):288-297 (2014).

As used herein, the term “hypervariable region” refers to the amino acid residues of an antibody which are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

As used herein, the terms “individual”, “patient”, or “subject” can be an individual organism, a vertebrate, a mammal, or a human. In some embodiments, the individual, patient or subject is a human.

As used herein, “Lo1+1+2” refers to a heterodimeric tetravalent multispecific antibody in which the Fab domains (a) bind to two distinct target epitopes that are within a proximity of 60-120 angstroms from each other (thus allowing simultaneous binding), and (b) have monovalent binding affinities or effective affinities (K_{D}) that range from about 100 nM to about 100 pM.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide”, “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. The term “specifically binds” may also refer to binding where a molecule (e.g., an antibody or antigen binding fragment thereof) binds to a particular polypeptide, or an epitope on a particular polypeptide, without substantially binding to any other polypeptide, or polypeptide epitope.

As used herein, the term “simultaneous” therapeutic use refers to the administration of at least two active ingredients by the same route and at the same time or at substantially the same time.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Heterodimeric Trivalent/Tetravalent Multispecific Antibodies of the Present Technology

The heterodimeric trivalent/tetravalent multispecific antibodies of the present technology can bind simultaneously to three or four targets that have a distinct structure, e.g., 3-4 different target antigens, 3-4 different epitopes on the same target antigen, or a combination of haptens and target antigens or epitopes on a target antigen. A variety of HDTVS antibodies can be produced using molecular engineering. For example, the HDTVS antibodies disclosed herein utilize combinations of the full immunoglobulin framework (e.g., IgG), and single chain variable fragments (scFvs).

HDTVS antibodies can be made, for example, by combining and/or engineering heavy chains and/or light chains that recognize different epitopes of the same or different antigen. In some embodiments, the HDTVS protein is trivalent and tri-specific, comprising, for example, an immunoglobulin (e.g., IgG) with a binding site for a first antigen (one V_{H}/V_{L} pair) and a binding site for a second antigen (a different V_{H}/V_{L} pair) and an scFv for a third antigen. In some embodiments, the HDTVS protein is trivalent and bispecific, comprising, for example, an immunoglobulin (e.g., IgG) with two binding sites (two V_{H}/V_{L} pairs) for a first antigen, and a scFv for a second antigen. In some embodiments, the HDTVS protein is tetravalent and tri-specific, comprising, for example, an immunoglobulin (e.g., IgG) with a binding site for a first antigen (one V_{H}/V_{L} pair) and a binding site for a second antigen (a different V_{H}/V_{L} pair) and two identical scFvs for a third antigen. In some embodiments, the HDTVS protein is tetravalent and tri-specific, comprising, for example, an immunoglobulin (e.g., IgG) with two binding sites (two V_{H}/V_{L} pairs) for a first antigen, an scFv for a second antigen and an scFv for a third antigen. In some embodiments, the HDTVS protein is tetravalent and tetra-specific, comprising, for example, an immunoglobulin (e.g., IgG) with a binding site for a first antigen (one V_{H}/V_{L} pair) and a binding site for a second antigen (different V_{H}/V_{L} pair), an scFv for a third antigen and an scFv for a fourth antigen.

In some embodiments, at least one scFv of the HDTVS antibodies of the present technology binds to an antigen or epitope of a B-cell, a T-cell, a myeloid cell, a plasma cell, or a mast-cell. Additionally or alternatively, in certain embodiments, at least one scFv of the HDTVS antibodies of the present technology binds to an antigen selected from the group consisting of Dabigatran, a4, a4b7, a4b7+aEb7, a5, AXL, BnDOTA, CD11a (LFA-1), CD3, CD4, CD8, CD16, CD19, CD22, CD23, CD25, CD28, CD30 (TNFRSF8), CD33, CD38, CD40, CD40L, CD47, CD49b (a2), CD54 (ICAM-1), CD56, CD74, CD80, CD115 (CSF1R), CD116a (CSF2Ra), CD123, CD134 (OX40), CD137 (41BB), CD152 (CTLA4), CD184 (CXCR4), CD192 (CCR2), CD194 (CCR4), CD195 (CCR5), CD223 (LAG-3), CD252 (OX40L), CD254 (RANKL), CD262 (DR5), CD27, CD200, CD221 (IGF1R), CD248, CD274 (PD-L1), CD275 (ICOS-L), CD278 (ICOS), CD279 (PD-1), CD319 (SLAMF7), CD371 (CLEC12A), MADCAM1, MT1-MMP (MMP14), NKG2A, NRP1,TIGIT, VSIR, KIRDL1/2/3, and KIR2DL2.

Additionally or alternatively, in certain embodiments, the HDTVS antibodies disclosed herein are capable of binding to cells (e.g., tumor cells) that express a cell surface antigen selected from the group consisting of a2b b3 (Glycoprotein IIb/IIIa), a4, a4b7, a4b7 +aEb7, a5, Activin receptor type-2B, ALK1, Alpha-synuclein, amyloid beta, APP, AXL, Blood Group A, CAIX, CCL-2, CD105 (endoglin), CD115 (CSF1R), CD116a (CSF2Ra), CD123, CD152 (CTLA4), CD184 (CXCR4), CD19, CD192 (CCR2), CD194 (CCR4), CD195 (CCR5), CD20, CD200, CD22, CD221 (IGF1R), CD248, CD25, CD25? (BAFF), CD26, CD262 (DR5), CD276 (B7H3), CD3, CD30 (TNFRSF8), CD319 (SLAMF7), CD33, CD332 (FGFR2), CD350 (FZD10), CD37, CD371 (CLEC12A), CD38, CD4, CD49b (a2), CD51 (a5), CD52, CD56, CD61 (a4b3), CD70, CD73 (NTSE), CD74, CEA, Claudin-18.2, cMET, CRLR, DLL3, DLL4, DNA/histone (H1) complex, EGFR, EpCAM, EGFR-HER3, EGFRvIII, EphA3, ERGT(GalNAc) Tn Antigen, FLT1, FOLR1, frizzled family receptor (FZD), Lewis Y, Lewis X, GCGR, GD2, GD2 α-acetyl, GD3, GM1, GM1 fucosyl, GM2, GPA33, GPNMB, GUCY2C, HER2, HER3, HGFR (cMET), IgHe, IGLF2, Kallikreins, LINGO1, LOXL2, Ly6/PLAUR domain-containing protein 3, MADCAM1, MAG, Mesothelin, MT1-MMP (MMP14), MUC1, Mucin SAC, NaPi2b, NeuGc-GM3, notch, NOTCH2/NOTCH3 receptors, oxLDL, P-selectin, PCSK9, PDGFRA, PDGFRa, phosphatidylserine, polysialic acid, PSMA, PVRL4, RGMA, CD240D Blood group D antigen, root plate-specific spondin 3, serum amyloid P component, STEAP-1, TACSTD2, TGFb, TWEAKR, TYRP1, VEGFR2, VSIR, CD171 (L1CAM), CD19, CD47, pMHC[NY-ESO1], pMHC[MART1], pMHC[MAGEA1], pMHC[Tyrosinase], pMHC[gp100], pMHC[MUC1], pMHC[tax], pMHC[WT-1], pMHC[EBNA-1], pMHC[LMP2], pMHC[hTERT], GPC3, CD80, CD23, and fibronectin extra domain-B.

Methods for producing the HDTVS antibodies of the present technology include engineered recombinant monoclonal antibodies which have additional cysteine residues so that they crosslink more strongly than the more common immunoglobulin isotypes. See, e.g., FitzGerald et al., Protein Eng. 10(10):1221-1225 (1997). HDTVS recombinant fusion proteins can be engineered by linking two or more different single-chain antibody or antibody fragment segments with the needed dual specificities. See, e.g., Coloma et al., Nature Biotech. 15:159-163 (1997).

Recombinant methods can be used to produce a variety of fusion proteins. In some embodiments, a HDTVS antibody according to the present technology comprises an immunoglobulin, which immunoglobulin comprises two heavy chains and two light chains, and two scFvs, wherein each scFv is linked to the C-terminal end of one of the two light chains of any immunoglobulin disclosed herein. In various embodiments, scFvs are linked to the light chains via a linker sequence. In some embodiments, a linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length.

In some embodiments, a linker is characterized in that it tends not to adopt a rigid three-dimensional structure, but rather provides flexibility to the polypeptide (e.g., first and/or second antigen binding sites). In some embodiments, a linker is employed in a HDTVS antibody described herein based on specific properties imparted to the HDTVS antibody such as, for example, an increase in stability. In some embodiments, a HDTVS antibody of the present technology comprises a G₄S linker (SEQ ID NO: 2508). In certain embodiments, a HDTVS antibody of the present technology comprises a (G₄S)ₙ linker, wherein n is 1, 2, 3, 4, 5, 6, 7, 8, 9, 10, 1 1, 12, 13, 14, 15 or more (SEQ ID NO: 2509).

Exemplary V_{H} and V_{L} amino acid sequences that may be employed in the HDTVS antibodies of the present technology are provided in Table 1.

TABLE 1

| | | SEQ | | SEQ | | SEQ | | SEQ | | SEQ | | SEQ | | SEQ | | SEQ |
|---|---|---|---|---|---|---|---|---|---|---|---|---|---|---|---|---|
| | | ID | V_{L} | ID | V_{L} | ID | V_{L} | ID | | ID | V_{H} | ID | V_{H} | ID | V_{H} | ID |
| Antigen | V_{L} | NO | CDR1 | NO | CDR2 | NO | CDR3 | NO | V_{H} | NO | CDR1 | NO | CDR2 | NO | CDR3 | NO |
| a2b b3 | DILMTQSPSSM | 1 | QGIS | 2 | YGT | 3 | VQY | 4 | EVQLQQSGAELV | 5 | GFNI | 6 | IDPA | 7 | VRPL | 8 |
| (Glyco- | SVSLGDTVSIT | | SN | | | | AQLP | | KPGASVKLSCTA | | KDT | | NGYT | | YDYY | |
| protein | CHASQGISSNI | | | | | | YT | | SGFNIKDTYVHW | | Y | | | | AMDY | |
| IIb/ | GWLQQKPGKS | | | | | | | | VKQRPEQGLEWI | | | | | | | |
| IIIa) | FMGLIYYGTN | | | | | | | | GRIDPANGYTKY | | | | | | | |
| | LVDGVPSRFS | | | | | | | | DPKFQGKATITA | | | | | | | |
| | GSGSGADYSL | | | | | | | | DTSSNTAYLQLS | | | | | | | |
| | TISSLDSEDFA | | | | | | | | SLTSEDTAVYYC | | | | | | | |
| | DYYCVQYAQ | | | | | | | | VRPLYDYYAMD | | | | | | | |
| | LPYTFGGGTK | | | | | | | | YWGQGTSVTVSS | | | | | | | |
| | LEIK | | | | | | | | | | | | | | | |
| a2b b3 | DIQMTQTPSTL | 9 | QDIN | 10 | YTS | 11 | QQG | 12 | QVQLVQSGAEV | 13 | GYA | 14 | IYPG | 15 | ARRD | 16 |
| (Glyco- | SASVGDRVTIS | | NY | | | | NTLP | | KKPGSSVKVSCK | | FTNY | | SGGT | | GNYG | |
| protein | CRASQDINNY | | | | | | WT | | ASGYAFTNYLIE | | L | | | | WFAY | |
| IIb/ | LNWYQQKPG | | | | | | | | WVRQAPGQGLE | | | | | | | |
| IIIa) | KAPKLLIYYTS | | | | | | | | WIGVIYPGSGGT | | | | | | | |
| | TLHSGVPSRFS | | | | | | | | NYNEKFKGRVTL | | | | | | | |
| | GSGSGTDYTL | | | | | | | | TVDESTNTAYME | | | | | | | |
| | TISSLQPDDFA | | | | | | | | LSSLRSEDTAVY | | | | | | | |
| | TYFCQQGNTL | | | | | | | | FCARRDGNYGWF | | | | | | | |
| | PWTFGQGTKV | | | | | | | | AYWGQGTLVTV | | | | | | | |
| | EVK | | | | | | | | SS | | | | | | | |
| a4 | DIQMTQSPSSL | 17 | QDIN | 18 | YTS | 19 | LQY | 20 | QVQLVQSGAEV | 21 | GFNI | 22 | IDPA | 23 | AREG | 24 |
| | SASVGDRVTIT | | KY | | | | DNL | | KKPGASVKVSCK | | KDT | | NGYT | | YYGN | |
| | CKTSQDINKY | | | | | | WT | | ASGFNIKDTYIH | | Y | | | | YGVY | |
| | MAWYQQTPG | | | | | | | | WVRQAPGQRLE | | | | | | AMDY | |
| | KAPRLLIHYTS | | | | | | | | WMGRIDPANGY | | | | | | | |
| | ALQPGIPSRFS | | | | | | | | TKYDPKFQGRVT | | | | | | | |
| | GSGSGRDYTF | | | | | | | | ITADTSASTAYM | | | | | | | |
| | TISSLQPEDIA | | | | | | | | ELSSLRSEDEAV | | | | | | | |
| | TYYCLQYDNL | | | | | | | | YYCAREGYYGN | | | | | | | |
| | WTFGQGTKVE | | | | | | | | YGVYAMDYWG | | | | | | | |
| | IK | | | | | | | | QGTLVTVSS | | | | | | | |
| a4b7 | DVVMTQSPLS | 25 | QSLA | 26 | GIS | 27 | LQGT | 28 | QVQLVQSGAEV | 29 | GYTF | 30 | IDPS | 31 | ARGG | 32 |
| | LPVTPGEPASI | | KSYG | | | | HQP | | KKPGASVKVSCK | | TSY | | ESNT | | YDGW | |
| | SCRSSQSLAKS | | NTY | | | | YT | | GSGYTFTSYWM | | W | | | | DYAI | |
| | YGNTYLSWYL | | | | | | | | HWVRQAPGQRL | | | | | | DY | |
| | QKPGQSPQLLI | | | | | | | | EWIGEIDPSESN | | | | | | | |
| | YGISNRFSGVP | | | | | | | | TNYNQKFKGRVT | | | | | | | |
| | DRFSGSGSGT | | | | | | | | LTVDISASTAYM | | | | | | | |
| | DFTLKISRVEA | | | | | | | | ELSSLRSEDTAV | | | | | | | |
| | EDVGVYYCLQ | | | | | | | | YYCARGGYDGWD | | | | | | | |
| | GTHQPYTFGQ | | | | | | | | YAIDYWGQGTL | | | | | | | |
| | GTKVEIK | | | | | | | | VTVSS | | | | | | | |
| a4b7 + | DIQMTQSPSSL | 33 | ESVD | 34 | YAS | 35 | QQG | 36 | EVQLVESGGGLV | 37 | GFFI | 38 | ISYS | 39 | ARTG | 40 |
| aEb7 | SASVGDRVTIT | | DL | | Q | | NSLP | | QPGGSLRLSCAA | | TNN | | GST | | SSGY | |
| | CRASESVDDL | | | | | | NT | | SGFFITNNYWGW | | Y | | | | FDF | |
| | LHWYQQKPG | | | | | | | | VRQAPGKGLEW | | | | | | | |
| | KAPKLLIKYAS | | | | | | | | VGYISYSGSTSY | | | | | | | |
| | QSISGVPSRFS | | | | | | | | NPSLKSRFTISR | | | | | | | |
| | GSGSGTDFTLT | | | | | | | | DTSKNTFYLQMN | | | | | | | |
| | ISSLQPEDFAT | | | | | | | | SLRAEDTAVYYC | | | | | | | |
| | YYCQQGNSLP | | | | | | | | ARTGSSGYFDFW | | | | | | | |
| | NTFGQGTKVE | | | | | | | | GQGTLVTVSS | | | | | | | |
| | IK | | | | | | | | | | | | | | | |
| a5 | EIVLTQSPATL | 41 | QSVS | 42 | DAS | 43 | QQRS | 44 | QVQLVESGGGV | 45 | GFTF | 46 | ISFD | 47 | AREA | 48 |
| | SLSPGERATLS | | SY | | | | NWP | | VQPGRSRRLSCA | | SRYT | | GSNK | | RGSY | |
| | CRASQSVSSY | | | | | | PFT | | ASGFTFSRYTMH | | | | | | AFDI | |
| | LAWYQQKPG | | | | | | | | WVRQAPGKGLE | | | | | | | |
| | QAPRLLIYDAS | | | | | | | | WVAVISFDGSNK | | | | | | | |
| | NRATGIPARFS | | | | | | | | YYVDSVKGRFTI | | | | | | | |
| | GSGSGTDFTLT | | | | | | | | SRDNSENTLYLQ | | | | | | | |
| | ISSLEPEDFAV | | | | | | | | VNILRAEDTAVY | | | | | | | |
| | YYCQQRSNWP | | | | | | | | YCAREARGSYAF | | | | | | | |
| | PFTFGPGTKV | | | | | | | | DIWGQGTMVTV | | | | | | | |
| | DIK | | | | | | | | SS | | | | | | | |
| Activin | QSALTQPASV | 49 | SSDV | 50 | GVS | 51 | GTFA | 52 | QVQLVQSGAEV | 53 | GYTF | 54 | INPV | 55 | ARGG | 56 |
| recep- | SGSPGQSITIS | | GSYN | | | | GGS | | KKPGASVKVSCK | | TSSY | | SGST | | WFDY | |
| tor | CTGTSSDVGSY | | Y | | | | YYG | | ASGYTFTSSYIN | | | | | | | |
| type-2B | NYVNWYQQH | | | | | | V | | WVRQAPGQGLE | | | | | | | |
| | PGKAPKLMIY | | | | | | | | WMGTINPVSGST | | | | | | | |
| | GVSKRPSGVS | | | | | | | | SYAQKFQGRVT | | | | | | | |
| | NRFSGSKSGN | | | | | | | | MTRDTSISTAYM | | | | | | | |
| | TASLTISGLQA | | | | | | | | ELSRLRSDDTAV | | | | | | | |
| | EDEADYYCGT | | | | | | | | YYCARGGWFDY | | | | | | | |
| | FAGGSYYGVF | | | | | | | | WGQGTLVTVSS | | | | | | | |
| | GGGTKLTVL | | | | | | | | | | | | | | | |
| ALK1 | EIVLTQSPGTL | 57 | QSVS | 58 | GTS | 59 | QQY | 60 | QVQLQESGPGLV | 61 | GGSI | 62 | IYYS | 63 | ARES | 64 |
| | SLSPGERATLS | | SSY | | | | GSSP | | KPSQTLSLTCTV | | SSGE | | GST | | VAGF | |
| | CRASQSVSSSY | | | | | | IT | | SGGSISSGEYYW | | YY | | | | DY | |
| | LAWYQQKPG | | | | | | | | NWIRQHPGKGLE | | | | | | | |
| | QAPRLLIYGTS | | | | | | | | WIGYIYYSGSTY | | | | | | | |
| | SRATGIPDRFS | | | | | | | | YNPSLKSRVTIS | | | | | | | |
| | GSGSGTDFTLT | | | | | | | | VDTSKNQFSLKL | | | | | | | |
| | ISRLEPEDFAV | | | | | | | | SSVTAADTAVYY | | | | | | | |
| | YYCQQYGSSPI | | | | | | | | CARESVAGFDYW | | | | | | | |
| | TFGQGTRLEIK | | | | | | | | GQGTLVTVSS | | | | | | | |
| Alpha- | DIQMTQSPSSL | 65 | QTLL | 66 | WAS | 67 | QQY | 68 | EVQLVESGGGLV | 69 | GFTF | 70 | ISSG | 71 | ARGG | 72 |
| synu- | SASVGDRVTIT | | YSSN | | | | YSYP | | QPGGSLRLSCAA | | SNY | | GGST | | AGID | |
| clein | CKSIQTLLYSS | | QKNY | | | | LT | | SGFTFSNYGMSW | | G | | | | YW | |
| | NQKNYLAWF | | | | | | | | VRQAPGKGLEW | | | | | | | |
| | QQKPGKAPKL | | | | | | | | VASISSGGGSTY | | | | | | | |
| | LIYWASIRKSG | | | | | | | | YPDNVKGRFTIS | | | | | | | |
| | VPSRFSGSGSG | | | | | | | | RDDAKNSLYLQM | | | | | | | |
| | TDFTLTISSLQ | | | | | | | | NSLRAEDTAVYY | | | | | | | |
| | PEDLATYYCQ | | | | | | | | CARGGAGIDYW | | | | | | | |
| | QYYSYPLTFG | | | | | | | | GQGTLVTVSS | | | | | | | |
| | GGTKLEIK | | | | | | | | | | | | | | | |
| amyloid | DVVMTQSPLS | 73 | QSLL | 74 | LYS | 75 | WQG | 76 | EVQLLESGGGLV | 77 | GFTF | 78 | IRSG | 79 | VRYD | 80 |
| beta | LPVTPGEPASI | | DSDG | | | | THFP | | QPGGSLRLSCAA | | SNY | | GGRT | | HYSG | |
| | SCKSSQSLLDS | | KTY | | | | RT | | SGFTFSNYGMSW | | G | | | | SSDY | |
| | DGKTYLNWLL | | | | | | | | VRQAPGKGLEW | | | | | | | |
| | QKPGQSPQRLI | | | | | | | | VASIRSGGGRTY | | | | | | | |
| | YLVSKLDSGV | | | | | | | | YSDNVKGRFTIS | | | | | | | |
| | PDRFSGSGSGT | | | | | | | | RDNSKNTLYLQ | | | | | | | |
| | DFTLKISRVEA | | | | | | | | MNSLRAEDTAV | | | | | | | |
| | EDVGVYYCW | | | | | | | | YYCVRYDHYSGS | | | | | | | |
| | QGTHFPRTFG | | | | | | | | SDYWGQGTLVT | | | | | | | |
| | QGTKVEIK | | | | | | | | VSS | | | | | | | |
| amyloid | DVVMTQSPLS | 81 | qSLI | 82 | KVS | 83 | SQST | 84 | EVQLVESGGGLV | 85 | GFTF | 86 | INSV | 87 | ASGD | 88 |
| beta | LPVTLGQPASI | | YSDG | | | | HVP | | QPGGSLRLSCAA | | SRYS | | GNST | | Y | |
| | SCRSSQSLIYS | | NAY | | | | WT | | SGFTFSRYSMSW | | | | | | | |
| | DGNAYLHWF | | | | | | | | VRQAPGKGLELV | | | | | | | |
| | LQKPGQSPRL | | | | | | | | AQINSVGNSTYY | | | | | | | |
| | LIYKVSNRFSG | | | | | | | | PDTVKGRFTISR | | | | | | | |
| | VPDRFSGSGS | | | | | | | | DNAKNTLYLQMN | | | | | | | |
| | GTDFTLKISRV | | | | | | | | SLRAEDTAVYYC | | | | | | | |
| | EAEDVGVYYC | | | | | | | | ASGDYWGQGTL | | | | | | | |
| | SQSTHVPWTF | | | | | | | | VTVSS | | | | | | | |
| | GQGTKVEIK | | | | | | | | | | | | | | | |
| amyloid | DIVMTQSPLSL | 89 | QSLV | 90 | KVS | 91 | SQST | 92 | EVQLVESGGGLV | 93 | GFTF | 94 | INSN | 95 | ASGD | 96 |
| beta | PVTPGEPASIS | | YSNG | | | | HVP | | QPGGSLRLSCAA | | SSYG | | GGST | | YW | |
| | CRSSQSLVYS | | DTY | | | | WT | | SGFTFSSYGMSW | | | | | | | |
| | NGDTYLHWY | | | | | | | | VRQAPGKGLELV | | | | | | | |
| | LQKPGQSPQL | | | | | | | | ASINSNGGSTYY | | | | | | | |
| | LIYKVSNRFSG | | | | | | | | PDSVKGRFTISR | | | | | | | |
| | VPDRFSGSGS | | | | | | | | DNAKNSLYLQMN | | | | | | | |
| | GTDFTLKISRV | | | | | | | | SLRAEDTAVYYC | | | | | | | |
| | EAEDVGVYYC | | | | | | | | ASGDYWGQGTT | | | | | | | |
| | SQSTHVPWTF | | | | | | | | VTVSS | | | | | | | |
| | GQGTKVEIK | | | | | | | | | | | | | | | |
| amyloid | DVVMTQSPLS | 97 | QSLL | 98 | QIS | 99 | LQGT | 100 | QVQLVQSGAEV | 101 | GYY | 102 | IDPA | 103 | ASLY | 104 |
| beta | LPVTLGQPASI | | YSD | | | | HYP | | KKPGASVKVSCK | | TEA | | TGNT | | SLPV | |
| | SCKSSQSLLYS | | AKTY | | | | VL | | ASGYYTEAYYIH | | YY | | | | Y | |
| | DAKTYLNWF | | | | | | | | WVRQAPGQGLE | | | | | | | |
| | QQRPGQSPRR | | | | | | | | WMGRIDPATGNT | | | | | | | |
| | LIYQISRLDPG | | | | | | | | KYAPRLQDRVT | | | | | | | |
| | VPDRFSGSGS | | | | | | | | MTRDTSTSTVYM | | | | | | | |
| | GTDFTLKISRV | | | | | | | | ELSSLRSEDTAV | | | | | | | |
| | EAEDVGVYYC | | | | | | | | YYCASLYSLPVY | | | | | | | |
| | LQGTHYPVLF | | | | | | | | WGQGTTVTVSS | | | | | | | |
| | GQGTRLEIK | | | | | | | | | | | | | | | |
| amyloid | DIQMTQSPSSL | 105 | QSIS | 106 | AAS | 107 | QQS | 108 | QVQLVESGGGV | 109 | GFAF | 110 | IWFD | 111 | ARDRG | 112 |
| beta | SASVGDRVTIT | | SY | | | | YSTP | | VQPGRSLRLSCA | | SSYG | | GTKK | | IGARR | |
| | CRASQSISSYL | | | | | | LT | | ASGFAFSSYGMH | | | | | | GPYYM | |
| | NWYQQKPGK | | | | | | | | WVRQAPGKGLE | | | | | | DV | |
| | APKLLIYAASS | | | | | | | | WVAVIWFDGTK | | | | | | | |
| | LQSGVPSRFSG | | | | | | | | KYYTDSVKGRFT | | | | | | | |
| | SGSGTDFTLTI | | | | | | | | ISRDNSKNTLYL | | | | | | | |
| | SSLQPEDFATY | | | | | | | | QMNTLRAEDTA | | | | | | | |
| | YCQQSYSTPL | | | | | | | | VYYCARDRGIGA | | | | | | | |
| | TFGGGTKVEI | | | | | | | | RRGPYYMDVWG | | | | | | | |
| | K | | | | | | | | KGTTVTVSS | | | | | | | |
| APP | DIVLTQSPATL | 113 | QSVS | 114 | GAS | 115 | LQIY | 116 | QVELVESGGGLV | 117 | GFTF | 118 | INAS | 119 | ARGKG | 120 |
| | SLSPGERATLS | | SSY | | | | NMPI | | QPGGSLRLSCAA | | SSYA | | TRT | | GNTH | |
| | CRASQSVSSSY | | | | | | T | | SGFTFSSYAMSW | | | | | | KPYG | |
| | LAWYQQKPG | | | | | | | | VRQAPGKGLEW | | | | | | YVRY | |
| | QAPRLLIYGAS | | | | | | | | VSAINASGTRTY | | | | | | FDV | |
| | SRATGVPARF | | | | | | | | YADSVKGRFTIS | | | | | | | |
| | SGSGSGTDFTL | | | | | | | | RDNSKNTLYLQ | | | | | | | |
| | TISSLEPEDFA | | | | | | | | MNSLRAEDTAV | | | | | | | |
| | TYYCLQIYNM | | | | | | | | YYCARGKGNTH | | | | | | | |
| | PITFGQGTKVE | | | | | | | | KPYGYVRYFDV | | | | | | | |
| | IK | | | | | | | | WGQGTLVTVSS | | | | | | | |
| AXL | EIVLTQSPGTL | 121 | QSVS | 122 | GAS | 123 | QQY | 124 | EVQLLESGGGLV | 125 | GFTF | 126 | TSGS | 127 | AKIWI | 128 |
| | SLSPGERATLS | | SSY | | | | GSSP | | QPGGSLRLSCAA | | SSYA | | GAST | | AFDI | |
| | CRASQSVSSSY | | | | | | YT | | SGFTFSSYAMNW | | | | | | | |
| | LAWYQQKPG | | | | | | | | VRQAPGKGLEW | | | | | | | |
| | QAPRLLIYGAS | | | | | | | | VSTTSGSGASTY | | | | | | | |
| | SRATGIPDRFS | | | | | | | | YADSVKGRFTIS | | | | | | | |
| | GSGSGTDFTLT | | | | | | | | RDNSKNTLYLQ | | | | | | | |
| | ISRLEPEDFAV | | | | | | | | MNSLRAEDTAV | | | | | | | |
| | YYCQQYGSSP | | | | | | | | YYCAKIWIAFDI | | | | | | | |
| | YTFGQGTKLEI | | | | | | | | WGQGTMVTVSS | | | | | | | |
| | K | | | | | | | | | | | | | | | |
| Blood | DIQMTQTTSSL | 129 | QDIN | 130 | YTS | 131 | QQG | 132 | QVQLQQPGAELV | 133 | GYN | 134 | IYPG | 135 | AGQY | 136 |
| group A | SASLGDRVTIS | | NY | | | | NTLP | | KPGTSVKLSCKA | | FTSY | | SGIT | | GNLW | |
| | CRASQDINNY | | | | | | WT | | SGYNFTSYWINW | | W | | | | FAY | |
| | LNWYQQKPD | | | | | | | | VKLRPGQGLEWI | | | | | | | |
| | GTVKLLIHYTS | | | | | | | | GDIYPGSGITNY | | | | | | | |
| | RLHSGVPSRFS | | | | | | | | NEKFKSKATLTV | | | | | | | |
| | GSGSGTDYSL | | | | | | | | DTSSSTAYMQLS | | | | | | | |
| | TISNLEQEDIA | | | | | | | | SLASEDSALYYC | | | | | | | |
| | TYFCQQGNTL | | | | | | | | AGQYGNLWFAYW | | | | | | | |
| | PWTFGGGTKL | | | | | | | | GQGTLVTVSS | | | | | | | |
| | EIK | | | | | | | | | | | | | | | |
| BnDOTA | QAVVIQESAL | 137 | TGAV | 138 | GHN | 139 | ALW | 140 | HVKLQESGPGLV | 141 | GFSL | 142 | IWSG | 143 | ARRG | 144 |
| | TTPPGETVTLT | | TASN | | | | YSD | | QPSQSLSLTCTV | | TDY | | GGT | | SYPY | |
| | CGSSTGAVTA | | Y | | | | HWV | | SGFSLTDYGVHW | | G | | | | NYFD | |
| | SNYANWVQE | | | | | | IGGG | | VRQSPGKGLEWL | | | | | | A | |
| | KPDHCFTGLIG | | | | | | | | GVIWSGGGTAYN | | | | | | | |
| | GHNNRPPGVP | | | | | | | | TALISRLNIYRD | | | | | | | |
| | ARFSGSLIGDK | | | | | | | | NSKNQVFLEMNS | | | | | | | |
| | AALTIAGTQTE | | | | | | | | LQAEDTAMYYCA | | | | | | | |
| | DEAIYFCALW | | | | | | | | RRGSYPYNYFDA | | | | | | | |
| | YSDHWVIGGG | | | | | | | | WGCGTTVTVSS | | | | | | | |
| | TRLTVL | | | | | | | | | | | | | | | |
| CAIX | DIVMTQSQRF | 145 | QNVV | 146 | SAS | 147 | QQY | 148 | DVKLVESGGGLV | 149 | GFTF | 150 | INSD | 151 | ARHR | 152 |
| | MSTTVGDRVS | | SA | | | | SNYP | | KLGGSLKLSCAA | | SNY | | GGIT | | SGYF | |
| | ITCKASQNVV | | | | | | WT | | SGFTFSNYYMSW | | Y | | | | SMDY | |
| | SAVAWYQQK | | | | | | | | VRQTPEKRLELV | | | | | | | |
| | PGQSPKLLIYS | | | | | | | | AAINSDGGITYY | | | | | | | |
| | ASNRYTGVPD | | | | | | | | LDTVKGRFTISR | | | | | | | |
| | RFTGSGSGTDF | | | | | | | | DNAKNTLYLQMS | | | | | | | |
| | TLTISNMQSED | | | | | | | | SLKSEDTALFYC | | | | | | | |
| | LADFFCQQYS | | | | | | | | ARHRSGYFSMDY | | | | | | | |
| | NYPWTFGGGT | | | | | | | | WGQGTSVTVSS | | | | | | | |
| | KLEIK | | | | | | | | | | | | | | | |
| CCL-2 | EIVLTQSPATL | 153 | QSVS | 154 | DAS | 155 | HQYI | 156 | QVQLVQSGAEV | 157 | GGTF | 158 | IIPI | 159 | ARYD | 160 |
| | SLSPGERATLS | | DAY | | | | QLHS | | KKPGSSVKVSCK | | SSYG | | FGTA | | GIYG | |
| | CRASQSVSDA | | | | | | FT | | ASGGTFSSYGIS | | | | | | ELDF | |
| | YLAWYQQKP | | | | | | | | WVRQAPGQGLE | | | | | | | |
| | GQAPRLLIYD | | | | | | | | WMGGIIPIFGTA | | | | | | | |
| | ASSRATGVPA | | | | | | | | NYAQKFQGRVTI | | | | | | | |
| | RFSGSGSGTDF | | | | | | | | TADESTSTAYME | | | | | | | |
| | TLTISSLEPED | | | | | | | | LSSLRSEDTAVY | | | | | | | |
| | FAVYYCHQYIQ | | | | | | | | YCARYDGIYGEL | | | | | | | |
| | LHSFTFGQGT | | | | | | | | DFWGQGTLVTVS | | | | | | | |
| | KVEIK | | | | | | | | S | | | | | | | |
| CD105 | QIVLSQSPAIL | 161 | SSVS | 162 | ATS | 163 | QQW | 164 | EVKLEESGGGLV | 165 | GFTF | 166 | IRSK | 167 | TRWR | 168 |
| (endo- | SASPGEKVTMT | | Y | | | | SSNP | | QPGGSMKLSCAA | | SDA | | ASNH | | RFFD | |
| glin) | CRASSSVSYM | | | | | | LT | | SGFTFSDAWMD | | W | | AT | | S | |
| | HWYQQKPGSS | | | | | | | | WVRQSPEKGLE | | | | | | | |
| | PKPWIYATSN | | | | | | | | WVAEIRSKASNH | | | | | | | |
| | LASGVPVRFS | | | | | | | | ATYYAESVKGRF | | | | | | | |
| | GSGSGTSYSLT | | | | | | | | TISRDDSKSSVY | | | | | | | |
| | ISRVEAEDAAT | | | | | | | | LQMNSLRAEDTG | | | | | | | |
| | YYCQQWSSNP | | | | | | | | IYYCTRWRRFFD | | | | | | | |
| | LTFGAGTKLE | | | | | | | | SWGQGTTLTVSS | | | | | | | |
| | LK | | | | | | | | | | | | | | | |
| CD115 | EIVLTQSPATL | 169 | QSVD | 170 | AAS | 171 | HLSN | 172 | QVQLVQSGAEV | 173 | GYTF | 174 | INPY | 175 | ARES | 176 |
| (CSF1R) | SLSPGERATLS | | YDGD | | | | EDLS | | KKPGSSVKVSCK | | TDN | | NGGT | | PYFS | |
| | CKASQSVDYD | | NY | | | | T | | ASGYTFTDNYMI | | Y | | | | NLYV | |
| | GDNYMNWYQ | | | | | | | | WVRQAPGQGLE | | | | | | MDYW | |
| | QKPGQAPRLLI | | | | | | | | WMGDINPYNGG | | | | | | | |
| | YAASNLESGIP | | | | | | | | TTFNQKFKGRVT | | | | | | | |
| | ARFSGSGSGT | | | | | | | | ITADKSTSTAYM | | | | | | | |
| | DFTLTISSLEP | | | | | | | | ELSSLRSEDTAV | | | | | | | |
| | EDFAVYYCHLS | | | | | | | | YYCARESPYFSN | | | | | | | |
| | NEDLSTFGGG | | | | | | | | LYVMDYWGQGT | | | | | | | |
| | TKVEIK | | | | | | | | LVTVSS | | | | | | | |
| CD116a | QSVLTQPPSVS | 177 | GSNI | 178 | HNN | 179 | ATVE | 180 | QVQLVQSGAEV | 181 | GYT | 182 | FDPE | 183 | AIVG | 184 |
| (CSF2 | GAPGQRVTISC | | GAPY | | | | AGLS | | KKPGASVKVSCK | | LTEL | | ENEI | | SFSP | |
| Ra) | TGSGSNIGAPY | | D | | | | GSV | | VSGYTLTELSIH | | S | | | | LTLG | |
| | DVSWYQQLPG | | | | | | | | WVRQAPGKGLE | | | | | | L | |
| | TAPKLLIYHN | | | | | | | | WMGGFDPEENEI | | | | | | | |
| | NKRPSGVPDR | | | | | | | | VYAQRFQGRVT | | | | | | | |
| | FSGSKSGTSAS | | | | | | | | MTEDTSTDTAY | | | | | | | |
| | LAITGLQAEDE | | | | | | | | MELSSLRSEDTA | | | | | | | |
| | ADYYCATVEA | | | | | | | | VYYCAIVGSFSP | | | | | | | |
| | GLSGSVFGGG | | | | | | | | LTLGLWGQGTMV | | | | | | | |
| | TKLTVL | | | | | | | | TVSS | | | | | | | |
| CD11a | DIQMTQSPSSL | 185 | KTISKY | 186 | SGS | 187 | QQH | 188 | EVQLVESGGGLV | 189 | GYSF | 190 | IHPS | 191 | ARGI | 192 |
| (LFA-1) | SASVGDRVTIT | | | | | | NEYP | | QPGGSLRLSCAA | | TGH | | DSET | | YFYG | |
| | CRASKTISKYL | | | | | | LT | | SGYSFTGHWMN | | W | | | | TTYF | |
| | AWYQQKPGK | | | | | | | | WVRQAPGKGLE | | | | | | DYW | |
| | APKLLIYSGST | | | | | | | | WVGMIHPSDSET | | | | | | | |
| | LQSGVPSRFSG | | | | | | | | RYNQKFKDRFTI | | | | | | | |
| | SGSGTDFTLTI | | | | | | | | SVDKSKNTLYLQ | | | | | | | |
| | SSLQPEDFATY | | | | | | | | MNSLRAEDTAV | | | | | | | |
| | YCQQHNEYPL | | | | | | | | YYCARGIYFYGT | | | | | | | |
| | TFGQGTKVEI | | | | | | | | TYFDYWGQGTL | | | | | | | |
| | K | | | | | | | | VTVSS | | | | | | | |
| CD123 | DFVMTQSPSS | 193 | QSLL | 194 | WAS | 195 | QND | 196 | EVQLQQSGPELV | 197 | GYTF | 198 | IIPS | 199 | TRSH | 200 |
| | LTVTAGEKVT | | NSGN | | | | YSYP | | KPGASVKMSCK | | TDY | | N | | LLRA | |
| | MSCKSSQSLL | | QKNY | | | | YT | | ASGYTFTDYYM | | Y | | GAT | | SWFA | |
| | NSGNQKNYLT | | | | | | | | KWVKQSHGKSL | | | | | | Y | |
| | WYLQKPGQPP | | | | | | | | EWIGDIIPSNGA | | | | | | | |
| | KLLIYWASTR | | | | | | | | TFYNQKFKGKAT | | | | | | | |
| | ESGVPDRFTGS | | | | | | | | LTVDRSSSTAYM | | | | | | | |
| | GSGTDFTLTIS | | | | | | | | HLNSLTSEDSAV | | | | | | | |
| | SVQAEDLAVY | | | | | | | | YYCTRSHLLRAS | | | | | | | |
| | YCQNDYSYPY | | | | | | | | WFAYWGQGTLVT | | | | | | | |
| | TFGGGTKLEIK | | | | | | | | VSA | | | | | | | |
| CD123 | QAVVTQEPSL | 201 | TGAV | 202 | GTN | 203 | ALW | 204 | EVQLVESGGGLV | 205 | GFTF | 206 | IRSK | 207 | VRHG | 208 |
| | TVSPGGTVTL | | TTSN | | | | YSNL | | QPGGSLRLSCAA | | STYA | | YNNY | | NFGN | |
| | TCRSSTGAVT | | Y | | | | WV | | SGFTFSTYAMNW | | | | AT | | SYVS | |
| | TSNYANWVQ | | | | | | | | VRQAPGKGLEW | | | | | | WFAY | |
| | QKPGQAPRGL | | | | | | | | VGRIRSKYNNYA | | | | | | | |
| | IGGTNKRAPW | | | | | | | | TYYADSVKDRFT | | | | | | | |
| | TPARFSGSLLG | | | | | | | | ISRDDSKNSLYL | | | | | | | |
| | GKAALTITGA | | | | | | | | QMNSLKTEDTAV | | | | | | | |
| | QAEDEADYYC | | | | | | | | YYCVRHGNFGNS | | | | | | | |
| | ALWYSNLWV | | | | | | | | YVSWFAYWGQGT | | | | | | | |
| | FGGGTKLTVL | | | | | | | | LVTVSS | | | | | | | |
| CD134 | DIQMTQSPSSL | 209 | QDIS | 210 | YTS | 211 | QQG | 212 | EVQLVQSGAEVK | 213 | GYTF | 214 | MYPD | 215 | VLAP | 216 |
| (OX40) | SASVGDRVTIT | | NY | | | | HTLP | | KPGASVKVSCKA | | TDSY | | NGDS | | RWYF | |
| | CRASQDISNYL | | | | | | PT | | SGYTFTDSYMSW | | | | | | SVW | |
| | NWYQQKPGK | | | | | | | | VRQAPGQGLEWI | | | | | | | |
| | APKLLIYYTSR | | | | | | | | GDMYPDNGDSS | | | | | | | |
| | LRSGVPSRFSG | | | | | | | | YNQKFRERVTIT | | | | | | | |
| | SGSGTDFTLTI | | | | | | | | RDTSTSTAYLEL | | | | | | | |
| | SSLQPEDFATY | | | | | | | | SSLRSEDTAVYY | | | | | | | |
| | YCQQGHTLPP | | | | | | | | CVLAPRWYFSVW | | | | | | | |
| | TFGQGTKVEI | | | | | | | | GQGTLVTVSS | | | | | | | |
| | K | | | | | | | | | | | | | | | |
| CD137 | SYELTQPPSVS | 217 | NIGD | 218 | QDK | 219 | ATYT | 220 | EVQLVQSGAEVK | 221 | GYSF | 222 | IYPG | 223 | ARGY | 224 |
| (41BB) | VSPGQTASITC | | QY | | | | GFGS | | KPGESLRISCKG | | STY | | DSYT | | GIFD | |
| | SGDNIGDQYA | | | | | | LAV | | SGYSFSTYWISW | | W | | | | Y | |
| | HWYQQKPGQ | | | | | | | | VRQMPGKGLEWM | | | | | | | |
| | SPVLVIYQDK | | | | | | | | GKIYPGDSYTNY | | | | | | | |
| | NRPSGIPERFS | | | | | | | | SPSFQGQVTISA | | | | | | | |
| | GSNSGNTATL | | | | | | | | DKSISTAYLQWS | | | | | | | |
| | TISGTQAMDE | | | | | | | | SLKASDTAMYYC | | | | | | | |
| | ADYYCATYTG | | | | | | | | ARGYGIFDYWGQ | | | | | | | |
| | FGSLAVFGGG | | | | | | | | GTLVTVSS | | | | | | | |
| | TKLTVL | | | | | | | | | | | | | | | |
| CD137 | EIVLTQSPATL | 225 | QSVS | 226 | DAS | 227 | QQRS | 228 | QVQLQQWGAGL | 229 | GGSF | 230 | INHG | 231 | ARDY | 232 |
| (41BB) | SLSPGERATLS | | SY | | | | NWP | | LKPSETLSLTCA | | SGY | | GYV | | GPGN | |
| | CRASQSVSSY | | | | | | PALT | | VYGGSFSGYYWS | | Y | | | | YDWY | |
| | LAWYQQKPG | | | | | | | | WIRQSPEKGLEW | | | | | | FDL | |
| | QAPRLLIYDAS | | | | | | | | IGEINHGGYVTY | | | | | | | |
| | NRATGIPARFS | | | | | | | | NPSLESRVTISV | | | | | | | |
| | GSGSGTDFTLT | | | | | | | | DTSKNQFSLKLS | | | | | | | |
| | ISSLEPEDFAV | | | | | | | | SVTAADTAVYYC | | | | | | | |
| | YYCQQRSNWP | | | | | | | | ARDYGPGNYDWY | | | | | | | |
| | PALTFCGGTK | | | | | | | | FDLWGRGTLVTV | | | | | | | |
| | VEIK | | | | | | | | SS | | | | | | | |
| CD152 | DIQMTQSPSSL | 233 | QSIN | 234 | AAS | 235 | QQY | 236 | QVQLVESGGGV | 237 | GFTF | 238 | IWYD | 239 | ARDP | 240 |
| (CTLA4) | SASVGDRVTIT | | SY | | | | YSTP | | VQPGRSLRLSCA | | SSYG | | GSNK | | RGAT | |
| | CRASQSINSYL | | | | | | FT | | ASGFTFSSYGMH | | | | | | LYYY | |
| | DWYQQKPGK | | | | | | | | WVRQAPGKGLE | | | | | | YYGM | |
| | APKLLIYAASS | | | | | | | | WVAVIWYDGSN | | | | | | DV | |
| | LQSGVPSRFSG | | | | | | | | KYYADSVKGRFT | | | | | | | |
| | SGSGTDFTLTI | | | | | | | | ISRDNSKNTLYL | | | | | | | |
| | SSLQPEDFATY | | | | | | | | QMNSLRAEDTA | | | | | | | |
| | YCQQYYSTPF | | | | | | | | VYYCARDPRGAT | | | | | | | |
| | TFGPGTKVEIK | | | | | | | | LYYYYYGMDVW | | | | | | | |
| | | | | | | | | | GQGTTVTVSS | | | | | | | |
| CD152 | EIVLTQSPGTL | 241 | QSVGS | 242 | GAF | 243 | QQY | 244 | QVQLVESGGGV | 245 | GFTF | 246 | ISYD | 247 | ARTG | 248 |
| (CTLA4) | SLSPGERATLS | | SY | | | | GSSP | | VQPGRSLRLSCA | | SSYT | | GNNK | | WLGP | |
| | CRASQSVGSS | | | | | | WT | | ASGFTFSSYTMH | | | | | | FDY | |
| | YLAWYQQKP | | | | | | | | WVRQAPGKGLE | | | | | | | |
| | GQAPRLLIYG | | | | | | | | WVTFISYDGNNK | | | | | | | |
| | AFSRATGIPDR | | | | | | | | YYADSVKGRFTI | | | | | | | |
| | FSGSGSGTDFT | | | | | | | | SRDNSKNTLYLQ | | | | | | | |
| | LTISRLEPEDF | | | | | | | | MNSLRAEDTAIY | | | | | | | |
| | AVYYCQQYGS | | | | | | | | YCARTGWLGPFD | | | | | | | |
| | SPWTFGQGTK | | | | | | | | YWGQGTLVTVSS | | | | | | | |
| | VEIK | | | | | | | | | | | | | | | |
| CD16 | DTVLTQSPASL | 249 | QSVD | 250 | TTS | 251 | QQS | 252 | QVTLKESGPGIL | 253 | GFSL | 254 | IWWD | 255 | AQIN | 256 |
| | AVSLGQRATIS | | FDGD | | | | NEDP | | QPSQTLSLTCSF | | RTSG | | DDK | | PAWF | |
| | CKASQSVDFD | | SF | | | | YT | | SGFSLRTSGMGV | | MG | | | | AY | |
| | GDSFMNWYQ | | | | | | | | GWIRQPSGKGLE | | | | | | | |
| | QKPGQPPKLLI | | | | | | | | WLAHIWWDDDKR | | | | | | | |
| | YTTSNLESGIP | | | | | | | | YNPALKSRLTIS | | | | | | | |
| | ARFSASGSGT | | | | | | | | KDTSSNQVFLKI | | | | | | | |
| | DFTLNIHPVEE | | | | | | | | ASVDTADTATYY | | | | | | | |
| | EDTATYYCQQ | | | | | | | | CAQINPAWFAYW | | | | | | | |
| | SNEDPYTFGG | | | | | | | | GQGTLVTVSA | | | | | | | |
| | GTKLEIK | | | | | | | | | | | | | | | |
| CD184 | DIQMTQSPSSL | 257 | QGIS | 258 | AAS | 259 | QQY | 260 | EVQLVESGGGLV | 261 | GFTF | 262 | ISSR | 263 | ARDY | 264 |
| (CXCR4) | SASVGDRVTIT | | SW | | | | NSYP | | QPGGSLRLSCAA | | SSYS | | SRTI | | GGQP | |
| | CRASQGISSW | | | | | | RT | | AGFTFSSYSMNW | | | | | | PYYY | |
| | LAWYQQKPE | | | | | | | | VRQAPGKGLEW | | | | | | YYGM | |
| | KAPKSLIYAAS | | | | | | | | VSYISSRSRTIY | | | | | | DV | |
| | SLQSGVPSRFS | | | | | | | | YADSVKGRFTIS | | | | | | | |
| | GSGSGTDFTLT | | | | | | | | RDNAKNSLYLQM | | | | | | | |
| | ISSLQPEDFVT | | | | | | | | NSLRDEDTAVYY | | | | | | | |
| | YYCQQYNSYP | | | | | | | | CARDYGGQPPYY | | | | | | | |
| | RTFGQGTKVEI | | | | | | | | YYYGMDVWGQ | | | | | | | |
| | K | | | | | | | | GTTVTVSS | | | | | | | |
| CD19 | DIQMTQTTSSL | 265 | QDISK | 266 | HTS | 267 | QQG | 268 | EVKLQESGPGLV | 269 | GVSL | 270 | IWGS | 271 | AKHY | 272 |
| | SASLGDRVTIS | | Y | | | | NTLP | | APSQSLSVTCTV | | PDY | | ETT | | YYGG | |
| | CRASQDISKYL | | | | | | YT | | SGVSLPDYGVSW | | G | | | | SYAM | |
| | NWYQQKPDG | | | | | | | | IRQPPRKGLEWL | | | | | | DY | |
| | TVKLLIYHTSR | | | | | | | | GVIWGSETTYYN | | | | | | | |
| | LHSGVPSRFSG | | | | | | | | SALKSRLTIIKD | | | | | | | |
| | SGSGTDYSLTI | | | | | | | | NSKSQVFLK | | | | | | | |
| | SNLEQE | | | | | | | | MNSLQTDDTAIY | | | | | | | |
| | DIATYFCQQG | | | | | | | | YCAKHYYYGGS | | | | | | | |
| | NTLPYTFGGG | | | | | | | | YAMDYWGQGTS | | | | | | | |
| | TKLEIK | | | | | | | | VTVSS | | | | | | | |
| CD19 | EIVLTQSPDFQ | 273 | ESVDT | 274 | EAS | 275 | QQS | 276 | EVQLVESGGGLV | 277 | GFTF | 278 | IYPG | 279 | ARSG | 280 |
| | SVTPKEKVTIT | | FGISF | | | | KEVP | | QPGGSLRLSCAA | | SSSW | | DGDT | | FITT | |
| | CRASESVDTF | | | | | | FT | | SGFTFSSSWMNW | | | | | | VRDF | |
| | GISFMNWFQQ | | | | | | | | VRQAPGKGLEW | | | | | | DY | |
| | KPDQSPKLLIH | | | | | | | | VGRIYPGDGDTN | | | | | | | |
| | EASNQGSGVP | | | | | | | | YNVKFKGRFTIS | | | | | | | |
| | SRFSGSGSGTD | | | | | | | | RDDSKNSLYLQM | | | | | | | |
| | FTLTINSLEAE | | | | | | | | NSLKTEDTAVYY | | | | | | | |
| | DAATYYCQQS | | | | | | | | CARSGFITTVRD | | | | | | | |
| | KEVPFTFGGG | | | | | | | | FDYWGQGTLVTV | | | | | | | |
| | TKVEIK | | | | | | | | SS | | | | | | | |
| CD19 | DIQMTQSPSSL | 281 | TDIS | 282 | YGS | 283 | GQG | 284 | QVQLQESGPGLV | 285 | GHSI | 286 | ISYS | 287 | ARSL | 288 |
| | SASVGDSVTIT | | SH | | | | NRLP | | KPSETLSLTCAV | | SHD | | GIT | | ARTT | |
| | CQASTDISSHL | | | | | | YT | | SGHSISHDHAWS | | HA | | | | AMDY | |
| | NWYQQKPGK | | | | | | | | WVRQPPGEGLEW | | | | | | | |
| | APELLIYYGSH | | | | | | | | IGFISYSGITNY | | | | | | | |
| | LLSGVPSRFSG | | | | | | | | NPSLQGRVTISR | | | | | | | |
| | SGSGTDFTFTI | | | | | | | | DNSKNTLYLQMN | | | | | | | |
| | SSLEAEDAAT | | | | | | | | SLRAEDTAVYYC | | | | | | | |
| | YYCGQGNRLP | | | | | | | | ARSLARTTAMDY | | | | | | | |
| | YTFGQGTKVE | | | | | | | | WGEGTLVTVSS | | | | | | | |
| | IE | | | | | | | | | | | | | | | |
| CD19 | EIVLTQSPATL | 289 | SSVS | 290 | DTS | 291 | FQGS | 292 | QVQLQESGPGLV | 293 | GGSI | 294 | IWWD | 295 | ARME | 296 |
| | SLSPGERATLS | | Y | | | | VYPF | | KPSQTLSLTCTV | | STSG | | DDK | | LWSY | |
| | CSASSSVSYM | | | | | | T | | SGGSISTSGMGV | | MG | | | | YFDY | |
| | HWYQQKPGQ | | | | | | | | GWIRQHPGKGLE | | | | | | | |
| | APRLLIYDTSK | | | | | | | | WIGHIWWDDDK | | | | | | | |
| | LASGIPARFSG | | | | | | | | RYNPALKSRVTI | | | | | | | |
| | SGSGTDFTLTI | | | | | | | | SVDTSKNQFSLK | | | | | | | |
| | SSLEPEDVAV | | | | | | | | LSSVTAADTAVY | | | | | | | |
| | YYCFQGSVYP | | | | | | | | YCARMELWSYYF | | | | | | | |
| | FTFGQGTKLEI | | | | | | | | DYWGQGTLVTV | | | | | | | |
| | K | | | | | | | | SS | | | | | | | |
| CD19 | DIQLTQSPASL | 297 | QSVD | 298 | DAS | 299 | QQST | 300 | QVQLQQSGAELV | 301 | GYA | 302 | IWPG | 303 | ARRE | 304 |
| | AVSLGQRATIS | | YDGD | | | | EDP | | RPGSSVKISCKA | | FSSY | | DGDT | | TTTV | |
| | CKASQSVDYD | | SY | | | | WT | | SGYAFSSYWMNW | | W | | | | GRYY | |
| | GDSYLNWYQ | | | | | | | | VKQRPGQGLEWI | | | | | | YAMD | |
| | QIPGQPPKLLI | | | | | | | | GQIWPGDGDTNY | | | | | | Y | |
| | YDASNLVSGIP | | | | | | | | NGKFKGKATLTA | | | | | | | |
| | PRFSGSGSGTD | | | | | | | | DESSSTAYMQLS | | | | | | | |
| | FTLNIHPVEKV | | | | | | | | SLASEDSAVYFC | | | | | | | |
| | DAATYHCQQS | | | | | | | | ARRETTTVGRYY | | | | | | | |
| | TEDPWTFGGG | | | | | | | | YAMDYWGQGTT | | | | | | | |
| | TKLEIK | | | | | | | | VTVSS | | | | | | | |
| CD19 | DIVMTQAAPSI | 305 | KSLL | 306 | RMS | 307 | MQH | 308 | QVQLQQSGPELI | 309 | GYTF | 310 | INPY | 311 | ARGT | 312 |
| | PVTPGESVSIS | | NSNG | | | | LEYP | | KPGASVKMSCK | | TSYV | | NDGT | | YYYG | |
| | CRSSKSLLNSN | | NTY | | | | LT | | ASGYTFTSYVMH | | | | | | SRVF | |
| | GNTYLYWFLQ | | | | | | | | WVKQKPGQGLE | | | | | | DY | |
| | RPGQSPQLLIY | | | | | | | | QIGYINPYNDGT | | | | | | | |
| | RMSNLASGVP | | | | | | | | KYNEKFKGKATL | | | | | | | |
| | DRFSGSGSGT | | | | | | | | TSDKSSTAYMEL | | | | | | | |
| | AFTLRISRVEA | | | | | | | | SSLTSEDSAVYY | | | | | | | |
| | EDVGVYYCM | | | | | | | | CARGTYYYGSRV | | | | | | | |
| | QHLEYPLTFG | | | | | | | | FDYWGQGTTLT | | | | | | | |
| | AGTKLEIK | | | | | | | | VTVSS | | | | | | | |
| CD19 | EIVLTQSPAIM | 313 | SGVN | 314 | DTS | 315 | HQR | 316 | QVQLVQPGAEV | 317 | GYTF | 318 | IDPS | 319 | ARGS | 320 |
| | SASPGERVTM | | Y | | | | GSYT | | VKPGASVKLSCK | | TSN | | DSYT | | NPYY | |
| | TCSASSGVNY | | | | | | | | TSGYTFTSNWMH | | W | | | | YAMD | |
| | MHWYQQKPG | | | | | | | | WVKQAPGQGLE | | | | | | Y | |
| | TSPRRWIYDTS | | | | | | | | WIGEIDPSDSYT | | | | | | | |
| | KLASGVPARF | | | | | | | | NYNQNFQGKAKL | | | | | | | |
| | SGSGSGTDYS | | | | | | | | TVDKSTSTAYME | | | | | | | |
| | LTISSMEPEDA | | | | | | | | VSSLRSDDTAVY | | | | | | | |
| | ATYYCHQRGS | | | | | | | | YCARGSNPYYYA | | | | | | | |
| | YTFGGGTKLEI | | | | | | | | MDYWGQGTSVT | | | | | | | |
| | K | | | | | | | | VSS | | | | | | | |
| CD192 | DVVMTQSPLS | 321 | QSLL | 322 | LVS | 323 | WQG | 324 | EVQLVESGGGLV | 325 | GFTF | 326 | IRTK | 327 | TTFY | 328 |
| (CCR2) | LPVTLGQPASI | | DSDG | | | | THFP | | KPGGSLRLSCAA | | SAY | | NNNY | | GNGV | |
| | SCKSSQSLLDS | | KTF | | | | YT | | SGFTFSAYAMN | | A | | AT | | W | |
| | DGKTFLNWFQ | | | | | | | | WVRQAPGKGLE | | | | | | | |
| | QRPGQSPRRLI | | | | | | | | WVGRIRTKNNN | | | | | | | |
| | YLVSKLDSGV | | | | | | | | YATYYADSVKD | | | | | | | |
| | PDRFSGSGSGT | | | | | | | | RFTISRDDSKNT | | | | | | | |
| | DFTLKISRVEA | | | | | | | | LYLQMNSLKTED | | | | | | | |
| | EDVGVYYCW | | | | | | | | TAVYYCTTFYGN | | | | | | | |
| | QGTHFPYTFG | | | | | | | | GVWGQGTLVTVS | | | | | | | |
| | QGTRLEIK | | | | | | | | S | | | | | | | |
| CD194 | DVLMTQSPLS | 329 | RNIV | 330 | KVS | 331 | FQGS | 332 | EVQLVESGGDLV | 333 | GFIF | 334 | ISSA | 335 | GRHS | 336 |
| (CCR4) | LPVTPGEPASI | | HING | | | | LLP | | QPGRSLRLSCAA | | SNY | | STYS | | DGNF | |
| | SCRSSRNIVHI | | DTY | | | | WT | | SGFIFSNYGMSW | | G | | | | AFGY | |
| | NGDTYLEWYL | | | | | | | | VRQAPGKGLEW | | | | | | | |
| | QKPGQSPQLLI | | | | | | | | VATISSASTYSYY | | | | | | | |
| | YKVSNRFSGV | | | | | | | | PDSVKGRFTISRD | | | | | | | |
| | PDRFSGSGSGT | | | | | | | | NAKNSLYLQMN | | | | | | | |
| | DFTLKISRVEA | | | | | | | | SLRVEDTALYYC | | | | | | | |
| | EDVGVYYCFQ | | | | | | | | GRHSDGNFAFGY | | | | | | | |
| | GSLLPWTFGQ | | | | | | | | WGQGTLVTVSS | | | | | | | |
| | GTKVEIK | | | | | | | | | | | | | | | |
| CD195 | DIVMTQSPLSL | 337 | QRLL | 338 | EVS | 339 | SQST | 340 | EVQLVESGGGLV | 341 | GYTF | 342 | IYPG | 343 | GSSF | 344 |
| (CCR5) | PVTPGEPASIS | | SSYG | | | | HVPL | | KPGGSLRLSCAA | | SNY | | GNYI | | GSN | |
| | CRSSQRLLSSY | | HTY | | | | T | | SGYTFSNYWIGW | | W | | | | YVFA | |
| | GHTYLHWYL | | | | | | | | VRQAPGKGLEWI | | | | | | WFTY | |
| | QKPGQSPQLLI | | | | | | | | GDIYPGGNYIRN | | | | | | W | |
| | YEVSNRFSGV | | | | | | | | NEKFKDKTTLSA | | | | | | | |
| | PDRFSGSGSGT | | | | | | | | DTSKNTAYLQM | | | | | | | |
| | DFTLKISRVEA | | | | | | | | NSLKTEDTAVYY | | | | | | | |
| | EDVGVYYCSQ | | | | | | | | CGSSFGSNYVFA | | | | | | | |
| | STHVPLTFGQ | | | | | | | | WFTYWGQGTLV | | | | | | | |
| | GTKVEIK | | | | | | | | TVSS | | | | | | | |
| CD20 | EIVLTQSPATL | 345 | QSVS | 346 | DAS | 347 | QQRS | 348 | EVQLVESGGGLV | 349 | GFTF | 350 | ISWN | 351 | AKDI | 352 |
| | SLSPGERATLS | | SY | | | | NWPI | | QPGRSLRLSCAA | | NDY | | SGSI | | QYGN | |
| | CRASQSVSSY | | | | | | T | | SGFTFNDYAMH | | A | | | | YYYG | |
| | LAWYQQKPG | | | | | | | | WVRQAPGKGLE | | | | | | MDV | |
| | QAPRLLIYDAS | | | | | | | | WVSTISWNSGSI | | | | | | | |
| | NRATGIPARFS | | | | | | | | GYADSVKGRFTI | | | | | | | |
| | GSGSGTDFTLT | | | | | | | | SRDNAKKSLYLQ | | | | | | | |
| | ISSLEPEDFAV | | | | | | | | MNSLRAEDTALY | | | | | | | |
| | YYCQQRSNWP | | | | | | | | YCAKDIQYGNYY | | | | | | | |
| | ITFGQGTRLEI | | | | | | | | YGMDVWGQGTT | | | | | | | |
| | K | | | | | | | | VTVSS | | | | | | | |
| CD20 | QIVLSQSPAILS | 353 | SSVS | 354 | APS | 355 | QQW | 356 | QAYLQQSGAELV | 357 | GYTF | 358 | IYPG | 359 | ARVV | 360 |
| | ASPGEKVTMT | | Y | | | | SFNP | | RPGASVKMSCKA | | TSYN | | NGDT | | YYSN | |
| | CRASSSVSYM | | | | | | PT | | SGYTFTSYNMH | | | | | | SYWY | |
| | HWYQQKPGSS | | | | | | | | WVKQTPRQGLE | | | | | | FDV | |
| | PKPWIYAPSNL | | | | | | | | WIGAIYPGNGDT | | | | | | | |
| | ASGVPARFSG | | | | | | | | SYNQKFKGKATL | | | | | | | |
| | SGSGTSYSLTI | | | | | | | | TVDKSSSTAYMQ | | | | | | | |
| | SRVEAEDAAT | | | | | | | | LSSLTSEDSAVYF | | | | | | | |
| | YYCQQWSFNP | | | | | | | | CARVVYYSNSY | | | | | | | |
| | PTFGAGTKLE | | | | | | | | WYFDVWGTGTT | | | | | | | |
| | LK | | | | | | | | VTVSGPSVFPLAP | | | | | | | |
| | | | | | | | | | SS | | | | | | | |
| CD20 | QIVLSQSPAILS | 361 | SSVS | 362 | ATS | 363 | QQW | 364 | QAYLQQSGAELV | 365 | GYTF | 366 | IYPG | 367 | ARYD | 368 |
| | ASPGEKVTMT | | Y | | | | TFNP | | RPGASVKMSCKA | | TSYN | | NGDT | | YNYA | |
| | CRASSSVSYM | | | | | | PT | | SGYTFTSYNMH | | | | | | MDY | |
| | HWYQQKPGSS | | | | | | | | WVKQTPRQGLE | | | | | | | |
| | PKPWIYATSN | | | | | | | | WIGGIYPGNGDT | | | | | | | |
| | LASGVPARFS | | | | | | | | SYNQKFKGKATL | | | | | | | |
| | GSGSGTSYSFT | | | | | | | | TVGKSSSTAYMQ | | | | | | | |
| | ISRVEAEDAAT | | | | | | | | LSSLTSEDSAVYF | | | | | | | |
| | YYCQQWTFNP | | | | | | | | CARYDYNYAMD | | | | | | | |
| | PTFGGGTRLEI | | | | | | | | YWGQGTSVTVSS | | | | | | | |
| | K | | | | | | | | | | | | | | | |
| CD20 | QIVLSQSPAILS | 369 | SSVS | 370 | ATS | 371 | QQW | 372 | QVQLQQPGAELV | 373 | GYTF | 374 | IYPG | 375 | ARST | 376 |
| | ASPGEKVTMT | | Y | | | | TSNP | | KPGASVKMSCK | | TSYN | | NGDT | | YYGG | |
| | CRASSSVSYIH | | | | | | PT | | ASGYTFTSYNMH | | | | | | DWYF | |
| | WFQQKPGSSP | | | | | | | | WVKQTPGRGLE | | | | | | NV | |
| | KPWIYATSNL | | | | | | | | WIGAIYPGNGDT | | | | | | | |
| | ASGVPVRFSG | | | | | | | | SYNQKFKGKATL | | | | | | | |
| | SGSGTSYSLTI | | | | | | | | TADKSSSTAYMQ | | | | | | | |
| | SRVEAEDAAT | | | | | | | | LSSLTSEDSAVY | | | | | | | |
| | YYCQQWTSNP | | | | | | | | YCARSTYYGGD | | | | | | | |
| | PTFGGGTKLEI | | | | | | | | WYFNVWGAGTT | | | | | | | |
| | K | | | | | | | | VTVSA | | | | | | | |
| CD20 | DIQLTQSPSSL | 377 | SSVS | 378 | ATS | 379 | QQW | 380 | QVQLQQSGAEV | 381 | GYTF | 382 | IYPG | 383 | ARST | 384 |
| | SASVGDRVTM | | Y | | | | TSNP | | KKPGSSVKVSCK | | TSYN | | NGDT | | YYGG | |
| | TCRASSSVSYI | | | | | | PT | | ASGYTFTSYNMH | | | | | | DWYF | |
| | HWFQQKPGK | | | | | | | | WVKQAPGQGLE | | | | | | DV | |
| | APKPWIYATS | | | | | | | | WIGAIYPGNGDT | | | | | | | |
| | NLASGVPVRF | | | | | | | | SYNQKFKGKATL | | | | | | | |
| | SGSGSGTDYT | | | | | | | | TADESTNTAYME | | | | | | | |
| | FTISSLQPEDIA | | | | | | | | LSSLRSEDTAFYY | | | | | | | |
| | TYYCQQWTSN | | | | | | | | CARSTYYGGDW | | | | | | | |
| | PPTFGGGTKLE | | | | | | | | YFDVWGQGTTV | | | | | | | |
| | IK | | | | | | | | TVSS | | | | | | | |
| CD20 | DIVMTQTPLSL | 385 | KSLL | 386 | QMS | 387 | AQN | 388 | QVQLVQSGAEV | 389 | GYA | 390 | IFPG | 391 | ARNV | 392 |
| | PVTPGEPASIS | | HSNG | | | | LELP | | KKPGSSVKVSCK | | FSYS | | DGDT | | FDGY | |
| | CRSSKSLLHSN | | ITY | | | | YT | | ASGYAFSYSWIN | | W | | | | WLVY | |
| | GITYLYWYLQ | | | | | | | | WVRQAPGQGLE | | | | | | | |
| | KPGQSPQLLIY | | | | | | | | WMGRIFPGDGDT | | | | | | | |
| | QMSNLVSGVP | | | | | | | | DYNGKFKGRVTI | | | | | | | |
| | DRFSGSGSGT | | | | | | | | TADKSTSTAYME | | | | | | | |
| | DFTLKISRVEA | | | | | | | | LSSLRSEDTAVY | | | | | | | |
| | EDVGVYYCA | | | | | | | | YCARNVFDGYW | | | | | | | |
| | QNLELPYTFG | | | | | | | | LVYWGQGTLVT | | | | | | | |
| | GGTKVEIK | | | | | | | | VSS | | | | | | | |
| CD200 | DIQMTQSPSSL | 393 | QDIN | 394 | RAN | 395 | LQY | 396 | QVQLQQSGSELK | 397 | GYSF | 398 | IDPY | 399 | GRSK | 400 |
| | SASIGDRVTIT | | SY | | | | DEFP | | KPGASVKISCKA | | TDYI | | YGSS | | RDYF | |
| | CKASQDINSY | | | | | | YT | | SGYSFTDYIILWV | | | | | | DYW | |
| | LSWFQQKPGK | | | | | | | | RQNPGKGLEWIG | | | | | | | |
| | APKLLIYRAN | | | | | | | | HIDPYYGSSNYN | | | | | | | |
| | RLVDGVPSRF | | | | | | | | LKFKGRVTITAD | | | | | | | |
| | SGSGSGTDYT | | | | | | | | QSTTTAYMELSS | | | | | | | |
| | LTISSLQPEDF | | | | | | | | LRSEDTAVYYCG | | | | | | | |
| | AVYYCLQYDE | | | | | | | | RSKRDYFDYWG | | | | | | | |
| | FPYTFGGGTK | | | | | | | | QGTTLTVSS | | | | | | | |
| | LEIK | | | | | | | | | | | | | | | |
| CD22 | DIQLTQSPSSL | 401 | QSVL | 402 | WAS | 403 | HQY | 404 | QVQLQESGAELS | 405 | GYTF | 406 | INPR | 407 | ARRD | 408 |
| | AVSAGENVTM | | YSAN | | | | LSSW | | KPGASVKMSCK | | TSY | | NDYT | | ITTF | |
| | SCKSSQSVLYS | | HKNY | | | | T | | ASGYTFTSYWLH | | W | | | | Y | |
| | ANHKNYLAW | | | | | | | | WIKQRPGQGLE | | | | | | | |
| | YQQKPGQSPK | | | | | | | | WIGYINPRNDYT | | | | | | | |
| | LLIYWASTRES | | | | | | | | EYNQNFKDKATL | | | | | | | |
| | GVPDRFTGSG | | | | | | | | TADKSSSTAYMQ | | | | | | | |
| | SGTDFTLTISR | | | | | | | | LSSLTSEDSAVY | | | | | | | |
| | VQVEDLAIYY | | | | | | | | YCARRDITTFYW | | | | | | | |
| | CHQYLSSWTF | | | | | | | | GQGTTLTVSS | | | | | | | |
| | GGGTKLEIK | | | | | | | | | | | | | | | |
| CD221 | DIQMTQFPSSL | 409 | QGIR | 410 | AAS | 411 | LQH | 412 | EVQLLESGGGLV | 413 | GFTF | 414 | ISGS | 415 | AKDL | 416 |
| (IGF1R) | SASVGDRVTIT | | ND | | | | NSYP | | QPGGSLRLSCTA | | SSYA | | GGTT | | GWSD | |
| | CRASQGIRND | | | | | | CS | | SGFTFSSYAMNW | | | | | | SYYY | |
| | LGWYQQKPG | | | | | | | | VRQAPGKGLEW | | | | | | YYGM | |
| | KAPKRLIYAA | | | | | | | | VSAISGSGGTTFY | | | | | | DV | |
| | SRLHRGVPSRF | | | | | | | | ADSVKGRFTISR | | | | | | | |
| | SGSGSGTEFTL | | | | | | | | DNSRTTLYLQMN | | | | | | | |
| | TISSLQPEDFA | | | | | | | | SLRAEDTAVYYC | | | | | | | |
| | TYYCLQHNSY | | | | | | | | AKDLGWSDSYY | | | | | | | |
| | PCSFGQGTKL | | | | | | | | YYYGMDVWGQ | | | | | | | |
| | EIK | | | | | | | | GTTVTVSS | | | | | | | |
| CD221 | DIQMTQSPSSL | 417 | QGIS | 418 | AKS | 419 | QQY | 420 | EVQLLQSGGGLV | 421 | GFM | 422 | ISGS | 423 | AKDF | 424 |
| (IGF1R) | SASLGDRVTIT | | SY | | | | WTFP | | QPGGSLRLSCAA | | FSRY | | GGAT | | YQIL | |
| | CRASQGISSYL | | | | | | LT | | SGFMFSRYPMH | | P | | | | TGNA | |
| | AWYQQKPGK | | | | | | | | WVRQAPGKGLE | | | | | | FDY | |
| | APKLLIYAKST | | | | | | | | WVGSISGSGGAT | | | | | | | |
| | LQSGVPSRFSG | | | | | | | | PYADSVKGRFTIS | | | | | | | |
| | SGSGTDFTLTI | | | | | | | | RDNSKNTLYLQ | | | | | | | |
| | SSLQPEDSATY | | | | | | | | MNSLRAEDTAV | | | | | | | |
| | YCQQYWTFPL | | | | | | | | YYCAKDFYQILT | | | | | | | |
| | TFGGGTKVEI | | | | | | | | GNAFDYWGQGT | | | | | | | |
| | K | | | | | | | | TVTVSS | | | | | | | |
| CD221 | QIVLTQSPAIM | 425 | SSVS | 426 | GTS | 427 | QQRS | 428 | EVQLQQSGPELV | 429 | GYSF | 430 | RINP | 431 | CAKS | 432 |
| (IGF1R) | SASPGEKVTIT | | Y | | | | SYPF | | KPGSSVKISCKAS | | TAY | | D | | TSYD | |
| | CSASSSVSYIH | | | | | | T | | GYSFTAYYMHW | | Y | | NGG | | YDGY | |
| | WFQQKPGTSP | | | | | | | | VKQSHGKSLEQI | | | | | | WFDV | |
| | KVWIYGTSNL | | | | | | | | SGRINPDNGGNS | | | | | | | |
| | ASGVPARFTG | | | | | | | | YNQFKFGKAILT | | | | | | | |
| | SGSGTSYSLTI | | | | | | | | VDKSSNTAYMEL | | | | | | | |
| | SRMEAEDAAT | | | | | | | | RSLTSEDSAVYY | | | | | | | |
| | YYCQQRSSYP | | | | | | | | CAKSTSYDYDGY | | | | | | | |
| | FTFGSGTKLEI | | | | | | | | WFDVWGAGTTV | | | | | | | |
| | K | | | | | | | | TVSS | | | | | | | |
| CD221 | SSELTQDPAVS | 433 | SLRS | 434 | GEN | 435 | KSRD | 436 | EVQLVQSGAEVK | 437 | GGTF | 438 | IIPI | 439 | ARAP | 440 |
| (IGF1R) | VALGQTVRIT | | YY | | | | GSG | | KPGSSVKVSCKA | | SSYA | | FGTA | | LRFL | |
| | CQGDSLRSYY | | | | | | QHL | | SGGTFSSYAISW | | | | | | EWST | |
| | ATWYQQKPG | | | | | | V | | VRQAPGQGLEW | | | | | | QDHY | |
| | QAPILVIYGEN | | | | | | | | MGGIIPIFGTANY | | | | | | YYYY | |
| | KRPSGIPDRFS | | | | | | | | AQKFQGRVTITA | | | | | | MDV | |
| | GSSSGNTASLT | | | | | | | | DKSTSTAYMELS | | | | | | | |
| | ITGAQAEDEA | | | | | | | | SLRSEDTAVYYC | | | | | | | |
| | DYYCKSRDGS | | | | | | | | ARAPLRFLEWST | | | | | | | |
| | GQHLVFGGGT | | | | | | | | QDHYYYYYMDV | | | | | | | |
| | KLTVL | | | | | | | | WGKGTTVTVSS | | | | | | | |
| CD221 | EIVLTQSPGTL | 441 | QSIG | 442 | YAS | 443 | HQSS | 444 | EVQLVQSGGGLV | 445 | GFTF | 446 | IDTR | 447 | ARLG | 448 |
| (IGF1R) | SVSPGERATLS | | SS | | | | RLPH | | KPGGSLRLSCAA | | SSFA | | GAT | | NFYY | |
| | CRASQSIGSSL | | | | | | T | | SGFTFSSFAMHW | | | | | | GMDV | |
| | HWYQQKPGQ | | | | | | | | VRQAPGKGLEWI | | | | | | | |
| | APRLLIKYASQ | | | | | | | | SVIDTRGATYYA | | | | | | | |
| | SLSGIPDRFSG | | | | | | | | DSVKGRFTISRD | | | | | | | |
| | SGSGTDFTLTI | | | | | | | | NAKNSLYLQMN | | | | | | | |
| | SRLEPEDFAV | | | | | | | | SLRAEDTAVYYC | | | | | | | |
| | YYCHQSSRLP | | | | | | | | ARLGNFYYGMD | | | | | | | |
| | HTFGQGTKVE | | | | | | | | VWGQGTTVTVSS | | | | | | | |
| | IK | | | | | | | | | | | | | | | |
| CD221 | EIVLTQSPATL | 449 | QSVS | 450 | DAS | 451 | QQRS | 452 | QVELVESGGGVV | 453 | GFTF | 454 | IWFD | 455 | AREL | 456 |
| (IGF1R) | SLSPGERATLS | | SY | | | | KWP | | QPGRSQRLSCAA | | SSYG | | GSST | | GRRY | |
| | CRASQSVSSY | | | | | | PWT | | SGFTFSSYGMHW | | | | | | FDL | |
| | LAWYQQKPG | | | | | | | | VRQAPGKGLEW | | | | | | | |
| | QAPRLLIYDAS | | | | | | | | VAIIWFDGSSTYY | | | | | | | |
| | KRATGIPARFS | | | | | | | | ADSVRGRFTISRD | | | | | | | |
| | GSGSGTDFTLT | | | | | | | | NSKNTLYLQMNS | | | | | | | |
| | ISSLEPEDFAV | | | | | | | | LRAEDTAVYFCA | | | | | | | |
| | YYCQQRSKWP | | | | | | | | RELGRRYFDLWG | | | | | | | |
| | PWTFGQGTKV | | | | | | | | RGTLVSVSS | | | | | | | |
| | ESK | | | | | | | | | | | | | | | |
| CD221 | DIVMTQSPLSL | 457 | QSIV | 458 | KVS | 459 | FQGS | 460 | QVQLQESGPGLV | 461 | GYSI | 462 | ISYD | 463 | ARYG | 464 |
| (IGF1R) | PVTPGEPASIS | | HSNG | | | | HVP | | KPSETLSLTCTVS | | TGG | | GTN | | RVFF | |
| | CRSSQSIVHSN | | NTY | | | | WT | | GYSITGGYLWN | | YL | | | | DY | |
| | GNTYLQWYL | | | | | | | | WIRQPPGKGLEW | | | | | | | |
| | QKPGQSPQLLI | | | | | | | | IGYISYDGTNNY | | | | | | | |
| | YKVSNRLYGV | | | | | | | | KPSLKDRVTISRD | | | | | | | |
| | PDRFSGSGSGT | | | | | | | | TSKNQFSLKLSSV | | | | | | | |
| | DFTLKISRVEA | | | | | | | | TAADTAVYYCA | | | | | | | |
| | EDVGVYYCFQ | | | | | | | | RYGRVFFDYWG | | | | | | | |
| | GSHVPWTFGQ | | | | | | | | QGTLVTVSS | | | | | | | |
| | GTKVEIK | | | | | | | | | | | | | | | |
| CD221 | DVVMTQSPLS | 465 | QSLL | 466 | LGS | 467 | MQG | 468 | QVQLQESGPGLV | 469 | GGSI | 470 | IYHS | 471 | ARWT | 472 |
| (IGF1R) | LPVTPGEPASI | | HSNG | | | | THW | | KPSGTLSLTCAVS | | SSSN | | GST | | GRTD | |
| | SCRSSQSLLHS | | YNY | | | | PLT | | GGSISSSNWWSW | | W | | | | AFDI | |
| | NGYNYLDWY | | | | | | | | VRQPPGKGLEWI | | | | | | | |
| | LQKPGQSPQL | | | | | | | | GEIYHSGSTNYN | | | | | | | |
| | LIYLGSNRASG | | | | | | | | PSLKSRVTISVDK | | | | | | | |
| | VPDRFSGSGS | | | | | | | | SKNQFSLKLSSVT | | | | | | | |
| | GTDFTLKISRV | | | | | | | | AADTAVYYCAR | | | | | | | |
| | EAEDVGVYYC | | | | | | | | WTGRTDAFDIW | | | | | | | |
| | MQGTHWPLTF | | | | | | | | GQGTMVTVSS | | | | | | | |
| | GQGTKVEIK | | | | | | | | | | | | | | | |
| CD223 | EIVLTQSPATL | 473 | QSIS | 474 | DAS | 475 | QQRS | 476 | QVQLQQWGAGL | 477 | GGSF | 478 | INHR | 479 | AFGY | 480 |
| (L4G-3) | SLSPGERATLS | | SY | | | | NWP | | LKPSETLSLTCAV | | SDY | | GST | | SDYE | |
| | CRASQSISSYL | | | | | | LT | | YGGSFSDYYWN | | Y | | | | YNWF | |
| | AWYQQKPGQ | | | | | | | | WIRQPPGKGLEW | | | | | | DP | |
| | APRLLIYDASN | | | | | | | | IGEINHRGSTNSN | | | | | | | |
| | RATGIPARFSG | | | | | | | | PSLKSRVTLSLDT | | | | | | | |
| | SGSGTDFTLTI | | | | | | | | SKNQFSLKLRSV | | | | | | | |
| | SSLEPEDFAVY | | | | | | | | TAADTAVYYCAF | | | | | | | |
| | YCQQRSNWPL | | | | | | | | GYSDYEYNWFD | | | | | | | |
| | TFGQGTNLEIK | | | | | | | | PWGQGTLVTVSS | | | | | | | |
| CD248 | DIQMTQSPSSL | 481 | QNVG | 482 | SAS | 483 | QQY | 484 | QVQLQESGPGLV | 485 | GYTF | 486 | INPY | 487 | ARRG | 488 |
| | SASVGDRVTIT | | TA | | | | TNYP | | RPSQTLSLTCTAS | | TDY | | DDDT | | NSYD | |
| | CRASQNVGTA | | | | | | MYT | | GYTFTDYVIHWV | | V | | | | GYFD | |
| | VAWLQQTPG | | | | | | | | KQPPGRGLEWIG | | | | | | YSMD | |
| | KAPKLLIYSAS | | | | | | | | YINPYDDDTTYN | | | | | | Y | |
| | NRYTGVPSRF | | | | | | | | QKFKGRVTMLV | | | | | | | |
| | SGSGSGTDYT | | | | | | | | DTSSNTAYLRLSS | | | | | | | |
| | FTISSLQPEDIA | | | | | | | | VTAEDTAVYYC | | | | | | | |
| | TYYCQQYTNY | | | | | | | | ARRGNSYDGYFD | | | | | | | |
| | PMYTFGQGTK | | | | | | | | YSMDYWGSGTP | | | | | | | |
| | VQIK | | | | | | | | VTVSS | | | | | | | |
| CD25 | QIVSTQSPAIM | 489 | SSRS | 490 | DTS | 491 | HQRS | 492 | QLQQSGTVLARP | 493 | GYSF | 494 | IYPG | 495 | SRDY | 496 |
| | SASPGEKVTM | | Y | | | | SYT | | GASVKMSCKAS | | TRY | | NSDT | | GYYF | |
| | TCSASSSRSY | | | | | | | | GYSFTRYWMHW | | W | | | | DF | |
| | MQWYQQKPG | | | | | | | | IKQRPGQGLEWI | | | | | | | |
| | TSPKRWIYDTS | | | | | | | | GAIYPGNSDTSY | | | | | | | |
| | KLASGVPARF | | | | | | | | NQKFEGKAKLTA | | | | | | | |
| | SGSGSGTSYSL | | | | | | | | VTSASTAYMELS | | | | | | | |
| | TISSMEAEDA | | | | | | | | SLTHEDSAVYYC | | | | | | | |
| | ATYYCHQRSS | | | | | | | | SRDYGYYFDFW | | | | | | | |
| | YTFGGGTKLEI | | | | | | | | GQGTTLTVSS | | | | | | | |
| | K | | | | | | | | | | | | | | | |
| CD25 | DIQMTQSPSTL | 497 | SSIS | 498 | TTS | 499 | HQRS | 500 | QVQLVQSGAEV | 501 | GYTF | 502 | INPS | 503 | ARGG | 504 |
| | SASVGDRVTIT | | Y | | | | TYPL | | KKPGSSVKVSCK | | TSYR | | TGYT | | GVFD | |
| | CSASSSISYMH | | | | | | T | | ASGYTFTSYRMH | | | | | | YW | |
| | WYQQKPGKA | | | | | | | | WVRQAPGQGLE | | | | | | | |
| | PKLLIYTTSNL | | | | | | | | WIGYINPSTGYTE | | | | | | | |
| | ASGVPARFSG | | | | | | | | YNQKFKDKATIT | | | | | | | |
| | SGSGTEFTLTI | | | | | | | | ADESTNTAYMEL | | | | | | | |
| | SSLQPDDFAT | | | | | | | | SSLRSEDTAVYY | | | | | | | |
| | YYCHQRSTYP | | | | | | | | CARGGGVFDYW | | | | | | | |
| | LTFGQGTKVE | | | | | | | | GQGTLVTVSS | | | | | | | |
| | VK | | | | | | | | | | | | | | | |
| CD252 | DIQMTQSPSSL | 505 | QDIS | 506 | YTS | 507 | QQG | 508 | QVQLQESGPGLV | 509 | GGSF | 510 | ISYN | 511 | ARYK | 512 |
| (OX40L) | SASVGDRVTIT | | NY | | | | SALP | | KPSQTLSLTCAV | | SSGY | | GIT | | YDYD | |
| | CRASQDISNYL | | | | | | WT | | YGGSFSSGYWN | | | | | | GGHA | |
| | NWYQQKPGK | | | | | | | | WIRKHPGKGLEY | | | | | | MDY | |
| | APKLLIYYTSK | | | | | | | | IGYISYNGITYHN | | | | | | | |
| | LHSGVPSRFSG | | | | | | | | PSLKSRITINRDTS | | | | | | | |
| | SGSGTDYTLTI | | | | | | | | KNQYSLQLNSVT | | | | | | | |
| | SSLQPEDFATY | | | | | | | | PEDTAVYYCARY | | | | | | | |
| | YCQQGSALPW | | | | | | | | KYDYDGGHAMD | | | | | | | |
| | TFGQGTKVEI | | | | | | | | YWGQGTLVTVSS | | | | | | | |
| | K | | | | | | | | | | | | | | | |
| CD254 | EIVLTQSPGTL | 513 | QSVR | 514 | GAS | 515 | QQY | 516 | EVQLLESGGGLV | 517 | GFTF | 518 | ITGS | 519 | AKDP | 520 |
| (RANKL) | SLSPGERATLS | | GRY | | | | GSSP | | QPGGSLRLSCAA | | SSYA | | GGST | | GTTV | |
| | CRASQSVRGR | | | | | | RT | | SGFTFSSYAMSW | | | | | | IMSW | |
| | YLAWYQQKP | | | | | | | | VRQAPGKGLEW | | | | | | FDP | |
| | GQAPRLLIYG | | | | | | | | VSGITGSGGSTY | | | | | | | |
| | ASSRATGIPDR | | | | | | | | YADSVKGRFTIS | | | | | | | |
| | FSGSGSGTDFT | | | | | | | | RDNSKNTLYLQ | | | | | | | |
| | LTISRLEPEDF | | | | | | | | MNSLRAEDTAV | | | | | | | |
| | AVFYCQQYGS | | | | | | | | YYCAKDPGTTVI | | | | | | | |
| | SPRTFGQGTK | | | | | | | | MSWFDPWGQGT | | | | | | | |
| | VEIK | | | | | | | | LVTVSS | | | | | | | |
| CD257 | EIVLTQSPATL | 521 | QSVS | 522 | DAS | 523 | QQRS | 524 | QVQLQQWGAGL | 525 | GGSF | 526 | INHS | 527 | ARGY | 528 |
| (BAFF) | SLSPGERATLS | | RY | | | | NWP | | LKPSETLSLTCAV | | SGY | | GST | | YDIL | |
| | CRASQSVSRY | | | | | | RT | | YGGSFSGYYWS | | Y | | | | TGYY | |
| | LAWYQQKPG | | | | | | | | WIRQPPGKGLEW | | | | | | YYFD | |
| | QAPRLLIYDAS | | | | | | | | IGEINHSGSTNYN | | | | | | Y | |
| | NRATGIPARFS | | | | | | | | PSLKSRVTISVDT | | | | | | | |
| | GSGSGTDSTLT | | | | | | | | SKNQFSLKLSSVT | | | | | | | |
| | ISSLEPEDFAV | | | | | | | | AADTAVYYCAR | | | | | | | |
| | YYCQQRSNWP | | | | | | | | GYYDILTGYYYY | | | | | | | |
| | RTFGQGTKVEI | | | | | | | | FDYWGQGTLVT | | | | | | | |
| | K | | | | | | | | VSS | | | | | | | |
| CD257 | SSELTQDPAVS | 529 | SLRS | 530 | GKN | 531 | SSRD | 532 | QVQLQQSGAEV | 533 | GGTF | 534 | IIPM | 535 | ARSR | 536 |
| (BAFF) | VALGQTVRVT | | YY | | | | SSGN | | KKPGSSVRVSCK | | NNN | | FGTA | | DLLL | |
| | CQGDSLRSYY | | | | | | HWV | | ASGGTFNNNAIN | | A | | | | FPHH | |
| | ASQYQQKPGQ | | | | | | | | WVRQAPGQGLE | | | | | | ALSP | |
| | APVLVIYGKN | | | | | | | | WMGGIIPMFGTA | | | | | | | |
| | NRPSGIPDRFS | | | | | | | | KYSQNFQGRVAI | | | | | | | |
| | GSSSGNTASLT | | | | | | | | TADESTGTASME | | | | | | | |
| | ITGAQAEDEA | | | | | | | | LSSLRSEDTAVY | | | | | | | |
| | DYYCSSRDSS | | | | | | | | YCARSRDLLLFP | | | | | | | |
| | GNHWVFGGG | | | | | | | | HHALSPWGRGT | | | | | | | |
| | TEL | | | | | | | | MVTVSS | | | | | | | |
| CD26 | QIVLTQSPAIM | 537 | SSVS | 538 | STS | 539 | QQRS | 540 | QVQLQQSGAELV | 541 | GYTF | 542 | IFPG | 543 | ARWT | 544 |
| | SASPGEKVTIT | | Y | | | | SYPN | | KPGASVKLSCKA | | RSY | | DGST | | VVGP | |
| | CSASSSVSYM | | | | | | T | | SGYTFRSYDINW | | D | | | | GYFD | |
| | NWFQQKPGTS | | | | | | | | VRQRPEQGLEWI | | | | | | V | |
| | PKLWIYSTSNL | | | | | | | | GWIFPGDGSTKY | | | | | | | |
| | ASGVPARFSG | | | | | | | | NEKFKGKATLTT | | | | | | | |
| | SGSGTSYSLTI | | | | | | | | DKSSSTAYMQLS | | | | | | | |
| | SRMEAEDAAT | | | | | | | | RLTSEDSAVYFC | | | | | | | |
| | YYCQQRSSYP | | | | | | | | ARWTVVGPGYF | | | | | | | |
| | NTFGGGTKLEI | | | | | | | | DVWGAGTTVTV | | | | | | | |
| | K | | | | | | | | SS | | | | | | | |
| CD262 | DIQMTQSPSSL | 545 | QDVG | 546 | WAS | 547 | QQY | 548 | EVQLVESGGGLV | 549 | GFTF | 550 | ISSG | 551 | ARRG | 552 |
| (DR5) | SASVGDRVTIT | | TA | | | | SSYR | | QPGGSLRLSCAA | | SSYV | | GSYT | | DSMI | |
| | CKASQDVGTA | | | | | | T | | SGFTFSSYVMSW | | | | | | TTDY | |
| | VAWYQQKPG | | | | | | | | VRQAPGKGLEW | | | | | | W | |
| | KAPKLLIYWA | | | | | | | | VATISSGGSYTY | | | | | | | |
| | STRHTGVPSRF | | | | | | | | YPDSVKGRFTISR | | | | | | | |
| | SGSGSGTDFTL | | | | | | | | DNAKNTLYLQM | | | | | | | |
| | TISSLQPEDFA | | | | | | | | NSLRAEDTAVYY | | | | | | | |
| | TYYCQQYSSY | | | | | | | | CARRGDSMITTD | | | | | | | |
| | RTFGQGTKVEI | | | | | | | | YWGQGTLVTVSS | | | | | | | |
| | K | | | | | | | | | | | | | | | |
| CD262 | SSELTQDPAVS | 553 | SLRS | 554 | GKN | 555 | NSRD | 556 | EVQLVQSGGGVE | 557 | GFTF | 558 | INWN | 559 | AKIL | 560 |
| (DR5) | VALGQTVRIT | | YY | | | | SSGN | | RPGGSLRLSCAA | | DDY | | GGST | | GAGR | |
| | CQGDSLRSYY | | | | | | HVV | | SGFTFDDYGMS | | G | | | | GWYF | |
| | ASWYQQKPG | | | | | | | | WVRQAPGKGLE | | | | | | DL | |
| | QAPVLVIYGK | | | | | | | | WVSGINWNGGS | | | | | | | |
| | NNRPSGIPDRF | | | | | | | | TGYADSVKGRVT | | | | | | | |
| | SGSSSGNTASL | | | | | | | | ISRDNAKNSLYL | | | | | | | |
| | TITGAQAEDE | | | | | | | | QMNSLRAEDTA | | | | | | | |
| | ADYYCNSRDS | | | | | | | | VYYCAKILGAGR | | | | | | | |
| | SGNHVVFGGG | | | | | | | | GWYFDLWGKGT | | | | | | | |
| | TKLTVL | | | | | | | | TVTVSS | | | | | | | |
| CD262 | EIVLTQSPGTL | 561 | QGIS | 562 | GAS | 563 | QQF | 564 | QVQLQESGPGLV | 565 | GGSI | 566 | IHNS | 567 | ARDR | 568 |
| (DR5) | SLSPGERATLS | | RSY | | | | GSSP | | KPSQTLSLTCTVS | | SSGD | | GTT | | GGDY | |
| | CRASQGISRSY | | | | | | WT | | GGSISSGDYFWS | | YF | | | | YYGM | |
| | LAWYQQKPG | | | | | | | | WIRQLPGKGLEW | | | | | | DV | |
| | QAPSLLIYGAS | | | | | | | | IGHIHNSGTTYYN | | | | | | | |
| | SRATGIPDRFS | | | | | | | | PSLKSRVTISVDT | | | | | | | |
| | GSGSGTDFTLT | | | | | | | | SKKQFSLRLSSVT | | | | | | | |
| | ISRLEPEDFAV | | | | | | | | AADTAVYYCAR | | | | | | | |
| | YYCQQFGSSP | | | | | | | | DRGGDYYYGMD | | | | | | | |
| | WTFGQGTKVE | | | | | | | | VWGQGTTVTVSS | | | | | | | |
| | IK | | | | | | | | | | | | | | | |
| CD27 | DIQMTQSPSSL | 569 | QGIS | 570 | AAS | 571 | QQY | 572 | QVQLVESGGGV | 573 | GFTF | 574 | IWYD | 575 | ARGSG | 576 |
| | SASVGDRVTIT | | RW | | | | NTYP | | VQPGRSLRLSCA | | SSYD | | GSNK | | NWGFF | |
| | CRASQGISRW | | | | | | RT | | ASGFTFSSYDMH | | | | | | DY | |
| | LAWYQQKPE | | | | | | | | WVRQAPGKGLE | | | | | | | |
| | KAPKSLIYAAS | | | | | | | | WVAVIWYDGSN | | | | | | | |
| | SLQSGVPSRFS | | | | | | | | KYYADSVKGRFT | | | | | | | |
| | GSGSGTDFTLT | | | | | | | | ISRDNSKNTLYL | | | | | | | |
| | ISSLQPEDFAT | | | | | | | | QMNSLRAEDTA | | | | | | | |
| | YYCQQYNTYP | | | | | | | | VYYCARGSGNW | | | | | | | |
| | RTFGQGTKVEI | | | | | | | | GFFDYWGQGTL | | | | | | | |
| | K | | | | | | | | VTVSS | | | | | | | |
| CD274 | QSALTQPASV | 577 | SSDV | 578 | DVS | 579 | SSYT | 580 | EVQLLESGGGLV | 581 | GFTF | 582 | IYPS | 583 | ARIK | 584 |
| (PD-L1) | SGSPGQSITISC | | GGYN | | | | SSST | | QPGGSLRLSCAA | | SSYI | | GGIT | | LGTV | |
| | TGTSSDVGGY | | Y | | | | RV | | SGFTFSSYIMMW | | | | | | TTVD | |
| | NYVSWYQQH | | | | | | | | VRQAPGKGLEW | | | | | | Y | |
| | PGKAPKLMIY | | | | | | | | VSSIYPSGGITFY | | | | | | | |
| | DVSNRPSGVS | | | | | | | | ADTVKGRFTISR | | | | | | | |
| | NRFSGSKSGN | | | | | | | | DNSKNTLYLQM | | | | | | | |
| | TASLTISGLQA | | | | | | | | NSLRAEDTAVYY | | | | | | | |
| | EDEADYYCSS | | | | | | | | CARIKLGTVTTV | | | | | | | |
| | YTSSSTRVFGT | | | | | | | | DYWGQGTLVTV | | | | | | | |
| | GTKVTVL | | | | | | | | SS | | | | | | | |
| CD274 | DIQMTQSPSSL | 585 | QDVST | 586 | SAS | 587 | QQY | 588 | EVQLVESGGGLV | 589 | GFTF | 590 | ISPY | 591 | ARRH | 592 |
| (PD-L1) | SASVGDRVTIT | | A | | | | LYHP | | QPGGSLRLSCAA | | SDS | | GGST | | WPGG | |
| | CRASQDVSTA | | | | | | AT | | SGFTFSDSWIHW | | W | | | | FDY | |
| | VAWYQQKPG | | | | | | | | VRQAPGKGLEW | | | | | | | |
| | KAPKLLIYSAS | | | | | | | | VAWISPYGGSTY | | | | | | | |
| | FLYSGVPSRFS | | | | | | | | YADSVKGRFTIS | | | | | | | |
| | GSGSGTDFTLT | | | | | | | | ADTSKNTAYLQ | | | | | | | |
| | ISSLQPEDFAT | | | | | | | | MNSLRAEDTAV | | | | | | | |
| | YYCQQYLYHP | | | | | | | | YYCARRHWPGG | | | | | | | |
| | ATFGQGTKVE | | | | | | | | FDYWGQGTLVT | | | | | | | |
| | IK | | | | | | | | VSS | | | | | | | |
| CD274 | EIVLTQSPGTL | 593 | QRVS | 594 | DAS | 595 | QQY | 596 | EVQLVESGGGLV | 597 | GFTF | 598 | IKQD | 599 | AREG | 600 |
| (PD-L1) | SLSPGERATLS | | SSY | | | | GSLP | | QPGGSLRLSCAA | | SRY | | GSEK | | GWFG | |
| | CRASQRVSSS | | | | | | WT | | SGFTFSRYWMS | | W | | | | ELAF | |
| | YLAWYQQKP | | | | | | | | WVRQAPGKGLE | | | | | | DY | |
| | GQAPRLLIYD | | | | | | | | WVANIKQDGSEK | | | | | | | |
| | ASSRATGIPDR | | | | | | | | YYVDSVKGRFTI | | | | | | | |
| | FSGSGSGTDFT | | | | | | | | SRDNAKNSLYLQ | | | | | | | |
| | LTISRLEPEDF | | | | | | | | MNSLRAEDTAV | | | | | | | |
| | AVYYCQQYGS | | | | | | | | YYCAREGGWFG | | | | | | | |
| | LPWTFGQGTK | | | | | | | | ELAFDYWGQGT | | | | | | | |
| | VEIK | | | | | | | | LVTVSS | | | | | | | |
| CD275 | DIQMTQSPSSL | 601 | QGIS | 602 | AAS | 603 | QQY | 604 | EVQLVESGGGLV | 605 | GFTF | 606 | IKQD | 607 | AREG | 608 |
| (ICOS-L) | SASVGDRVTIT | | NW | | | | DSYP | | QPGGSLRLSCAA | | SSY | | GNEK | | ILWF | |
| | CRASQGISNW | | | | | | RT | | SGFTFSSYWMSW | | W | | | | GDLP | |
| | LAWYQQKPE | | | | | | | | VRQAPGKGLEW | | | | | | TF | |
| | KAPKSLIYAAS | | | | | | | | VAYIKQDGNEKY | | | | | | | |
| | SLQSGVPSRFS | | | | | | | | YVDSVKGRFTIS | | | | | | | |
| | GSGSGTDFTLT | | | | | | | | RDNAKNSLYLQ | | | | | | | |
| | ISSLQPEDFAT | | | | | | | | MNSLRAEDTAV | | | | | | | |
| | YYCQQYDSYP | | | | | | | | YYCAREGILWFG | | | | | | | |
| | RTFGQGTKVEI | | | | | | | | DLPTFWGQGTLV | | | | | | | |
| | K | | | | | | | | TVSS | | | | | | | |
| CD276 | DIQLTQSPSFL | 609 | QNVD | 610 | SAS | 611 | QQY | 612 | EVQLVESGGGLV | 613 | GFTF | 614 | ISSD | 615 | GRGR | 616 |
| (B7H3) | SASVGDRVTIT | | TN | | | | NNY | | QPGGSLRLSCAA | | SSFG | | SSAI | | ENIY | |
| | CKASQNVDTN | | | | | | PFT | | SGFTFSSFGMHW | | | | | | YGSR | |
| | VAWYQQKPG | | | | | | | | VRQAPGKGLEW | | | | | | LDY | |
| | KAPKALIYSAS | | | | | | | | VAYISSDSSAIYY | | | | | | | |
| | YRYSGVPSRFS | | | | | | | | ADTVKGRFTISR | | | | | | | |
| | GSGSGTDFTLT | | | | | | | | DNAKNSLYLQM | | | | | | | |
| | ISSLQPEDFAT | | | | | | | | NSLRDEDTAVYY | | | | | | | |
| | YYCQQYNNYP | | | | | | | | CGRGRENIYYGS | | | | | | | |
| | FTFGQGTKLEI | | | | | | | | RLDYWGQGTTV | | | | | | | |
| | K | | | | | | | | TVSS | | | | | | | |
| CD276 | DIVMTQSPAT | 617 | QSIS | 618 | YAS | 619 | QNG | 620 | QVQLQQSGAELV | 621 | GYTF | 622 | IFPG | 623 | ARQT | 624 |
| (B7H3) | LSVTPGDRVS | | DY | | | | HSFP | | KPGASVKLSCKA | | TNY | | DGST | | TATW | |
| | LSCRASQSISD | | | | | | LT | | SGYTFTNYDINW | | D | | | | FAY | |
| | YLHWYQQKS | | | | | | | | VRQRPEQGLEWI | | | | | | | |
| | HESPRLLIKYA | | | | | | | | GWIFPGDGSTQY | | | | | | | |
| | SQSISGIPSRFS | | | | | | | | NEKFKGKATLTT | | | | | | | |
| | GSGSGSDFTLS | | | | | | | | DTSSSTAYMQLS | | | | | | | |
| | INSVEPEDVGV | | | | | | | | RLTSEDSAVYFC | | | | | | | |
| | YYCQNGHSFP | | | | | | | | ARQTTATWFAY | | | | | | | |
| | LTFGAGTKLE | | | | | | | | WGQGTLVTVSA | | | | | | | |
| | LK | | | | | | | | | | | | | | | |
| CD279 | DIQMTQSPSSL | 625 | LSIN | 626 | AAS | 627 | QQSS | 628 | EVQLLESGGVLV | 629 | GFTF | 630 | ISGG | 631 | VKWG | 632 |
| (PD-1) | SASVGDSITIT | | TF | | | | NTPF | | QPGGSLRLSCAA | | GSNF | | RDT | | NIYF | |
| | CRASLSINTFL | | | | | | T | | SGFTFSNFGMTW | | G | | DY | | | |
| | NWYQQKPGK | | | | | | | | VRQAPGKGLEW | | | | | | | |
| | APNLLIYAASS | | | | | | | | VSGISGGGRDTY | | | | | | | |
| | LHGGVPSRFS | | | | | | | | FADSVKGRFTISR | | | | | | | |
| | GSGSGTDFTLT | | | | | | | | DNSKNTLYLQM | | | | | | | |
| | IRTLQPEDFAT | | | | | | | | NSLKGEDTAVYY | | | | | | | |
| | YYCQQSSNTP | | | | | | | | CVKWGNIYFDY | | | | | | | |
| | FTFGPGTVVD | | | | | | | | WGQGTLVTVSS | | | | | | | |
| | FR | | | | | | | | | | | | | | | |
| CD279 | DIQMTQSPSSL | 633 | QTIG | 634 | TAT | 635 | QQV | 636 | EVQLVESGGGLV | 637 | GFTF | 638 | ISGG | 639 | ARQL | 640 |
| (PD-1) | SASVGDRVTIT | | TW | | | | YSIP | | QPGGSLRLSCAA | | SSY | | GANT | | YYFD | |
| | CLASQTIGTW | | | | | | WT | | SGFTFSSYMMSW | | M | | | | Y | |
| | LTWYQQKPG | | | | | | | | VRQAPGKGLEW | | | | | | | |
| | KAPKLLIYTAT | | | | | | | | VATISGGGANTY | | | | | | | |
| | SLADGVPSRFS | | | | | | | | YPDSVKGRFTISR | | | | | | | |
| | GSGSGTDFTLT | | | | | | | | DNAKNSLYLQM | | | | | | | |
| | ISSLQPEDFAT | | | | | | | | NSLRAEDTAVYY | | | | | | | |
| | YYCQQVYSIP | | | | | | | | CARQLYYFDYW | | | | | | | |
| | WTFGGGTKVE | | | | | | | | GQGTTVTVSS | | | | | | | |
| | IK | | | | | | | | | | | | | | | |
| CD279 | EIVLTQSPATL | 641 | QSVS | 642 | DAS | 643 | QQSS | 644 | QVQLVESGGGV | 645 | GITF | 646 | IWYD | 647 | ATND | 648 |
| (PD-1) | SLSPGERATLS | | SY | | | | NWP | | VQPGRSLRLDCK | | SNSG | | GSKR | | DY | |
| | CRASQSVSSY | | | | | | RT | | ASGITFSNSGMH | | | | | | | |
| | LAWYQQKPG | | | | | | | | WVRQAPGKGLE | | | | | | | |
| | QAPRLLIYDAS | | | | | | | | WVAVIWYDGSK | | | | | | | |
| | NRATGIPARFS | | | | | | | | RYYADSVKGRFT | | | | | | | |
| | GSGSGTDFTLT | | | | | | | | ISRDNSKNTLFLQ | | | | | | | |
| | ISSLEPEDFAV | | | | | | | | MNSLRAEDTAV | | | | | | | |
| | YYCQQSSNWP | | | | | | | | YYCATNDDYWG | | | | | | | |
| | RTFGQGTKVEI | | | | | | | | QGTLVTVSS | | | | | | | |
| | K | | | | | | | | | | | | | | | |
| CD279 | DIQMTQSPSSV | 649 | QGIS | 650 | AAS | 651 | QQA | 652 | QVQLVQSGAEV | 653 | GGTF | 654 | IIPM | 655 | ARAE | 656 |
| (PD-1) | SASVGDRVTIT | | SW | | | | NHLP | | KKPGSSVKVSCK | | SSYA | | FDTA | | HSST | |
| | CRASQGISSW | | | | | | FT | | ASGGTFSSYAIS | | | | | | GTFD | |
| | LAWYQQKPG | | | | | | | | WVRQAPGQGLE | | | | | | Y | |
| | KAPKLLISAAS | | | | | | | | WMGLIIPMFDTA | | | | | | | |
| | SLQSGVPSRFS | | | | | | | | GYAQKFQGRVAI | | | | | | | |
| | GSGSGTDFTLT | | | | | | | | TVDESTSTAYME | | | | | | | |
| | ISSLQPEDFAT | | | | | | | | LSSLRSEDTAVY | | | | | | | |
| | YYCQQANHLP | | | | | | | | YCARAEHSSTGT | | | | | | | |
| | FTFGGGTKVEI | | | | | | | | FDYWGQGTLVT | | | | | | | |
| | K | | | | | | | | VSS | | | | | | | |
| CD279 | QPVLTQPLSVS | 657 | NIGS | 658 | RDS | 659 | QVW | 660 | QVQLVQSGGGL | 661 | GFTF | 662 | IDTG | 663 | ARDE | 664 |
| (PD-1) | VALGQTARIT | | KN | | | | DSST | | VQPGGSLRLSCA | | SSY | | GGRT | | GGGT | |
| | CGGNNIGSKN | | | | | | AV | | ASGFTFSSYWMY | | W | | | | GWGV | |
| | VHWYQQKPG | | | | | | | | WVRQVPGKGLE | | | | | | LKDW | |
| | QAPVLVIYRD | | | | | | | | WVSAIDTGGGRT | | | | | | PYGL | |
| | SNRPSGIPERF | | | | | | | | YYADSVKGRFAI | | | | | | DA | |
| | SGSNSGNTAT | | | | | | | | SRVNAKNTMYL | | | | | | | |
| | LTISRAQAGDE | | | | | | | | QMNSLRAEDTA | | | | | | | |
| | ADYYCQVWD | | | | | | | | VYYCARDEGGG | | | | | | | |
| | SSTAVFGTGT | | | | | | | | TGWGVLKDWPY | | | | | | | |
| | KLTVL | | | | | | | | GLDAWGQGTLV | | | | | | | |
| | | | | | | | | | TVSS | | | | | | | |
| CD279 | EIVLTQSPATL | 665 | KGVS | 666 | LAS | 667 | QHSR | 668 | QVQLVQSGVEV | 669 | GYTF | 670 | INPS | 671 | ARRD | 672 |
| (PD-1) | SLSPGERATLS | | TSGY | | | | DLPL | | KKPGASVKVSCK | | TNY | | NGGT | | YRFD | |
| | CRASKGVSTS | | SY | | | | T | | ASGYTFTNYYM | | Y | | | | MGFD | |
| | GYSYLHWYQ | | | | | | | | YWVRQAPGQGL | | | | | | YW | |
| | QKPGQAPRLLI | | | | | | | | EWMGGINPSNGG | | | | | | | |
| | YLASYLESGV | | | | | | | | TNFNEKFKNRVT | | | | | | | |
| | PARFSGSGSGT | | | | | | | | LTTDSSTTTAYM | | | | | | | |
| | DFTLTISSLEPE | | | | | | | | ELKSLQFDDTAV | | | | | | | |
| | DFAVYYCQHS | | | | | | | | YYCARRDYRFD | | | | | | | |
| | RDLPLTFGGG | | | | | | | | MGFDYWGQGTT | | | | | | | |
| | TKVEIK | | | | | | | | VTVSS | | | | | | | |
| CD279 | EIVLTQSPATL | 673 | QSVR | 674 | DAS | 675 | QQR | 676 | VQLVQSGAEVK | 677 | GGTF | 678 | IIPI | 679 | ARPG | 680 |
| (PD-1) | SLSPGERATLS | | SY | | | | NYW | | KPGSSVKVSCKA | | SSYA | | FDTA | | LAAA | |
| | CRASQSVRSY | | | | | | PLT | | SGGTFSSYAISW | | | | | | YDTG | |
| | LAWYQQKPG | | | | | | | | VRQAPGQGLEW | | | | | | SLDY | |
| | QAPRLLIYDAS | | | | | | | | MGGIIPIFDTANY | | | | | | | |
| | NRATGIPARFS | | | | | | | | AQKFQGRVTITA | | | | | | | |
| | GSGSGTDFTLT | | | | | | | | DESTSTAYMELS | | | | | | | |
| | ISSLEPEDFAV | | | | | | | | SLRSEDTAVYYC | | | | | | | |
| | YYCQQRNYW | | | | | | | | ARPGLAAAYDTG | | | | | | | |
| | PLTFGQGTKV | | | | | | | | SLDYWGQGTLV | | | | | | | |
| | EIK | | | | | | | | TVSS | | | | | | | |
| CD3 | DIQLTQSPAIM | 681 | SSVS | 682 | DTS | 683 | QQW | 684 | DIKLQQSGAELA | 685 | GYTF | 686 | INPS | 687 | ARYY | 688 |
| | SASPGEKVTM | | Y | | | | SSNP | | RPGASVKMSCKT | | TRYT | | RGYT | | DDHY | |
| | TCRASSSVSY | | | | | | LT | | SGYTFTRYTMH | | | | | | CLDY | |
| | MNWYQQKSG | | | | | | | | WVKQRPGQGLE | | | | | | | |
| | TSPKRWIYDTS | | | | | | | | WIGYINPSRGYT | | | | | | | |
| | KVASGVPYRF | | | | | | | | NYNQKFKDKAT | | | | | | | |
| | SGSGSGTSYSL | | | | | | | | LTTDKSSSTAYM | | | | | | | |
| | TISSMEAEDA | | | | | | | | QLSSLTSEDSAV | | | | | | | |
| | ATYYCQQWSS | | | | | | | | YYCARYYDDHY | | | | | | | |
| | NPLTFGAGTK | | | | | | | | CLDYWGQGTTL | | | | | | | |
| | LELK | | | | | | | | TVSS | | | | | | | |
| CD3 | DIQLTQPNSVS | 689 | SGNI | 690 | DDD | 691 | HSY | 692 | EVQLLESGGGLV | 693 | GFTF | 694 | ISTS | 695 | AKFR | 696 |
| | TSLGSTVKLSC | | ENNY | | | | VSSF | | QPGGSLRLSCAA | | SSFP | | GGRT | | QYSG | |
| | TLSSGNIENNY | | | | | | NV | | SGFTFSSFPMAW | | | | | | GFDY | |
| | VHWYQLYEG | | | | | | | | VRQAPGKGLEW | | | | | | | |
| | RSPTTMIYDD | | | | | | | | VSTISTSGGRTYY | | | | | | | |
| | DKRPDGVPDR | | | | | | | | RDSVKGRFTISRD | | | | | | | |
| | FSGSIDRSSNS | | | | | | | | NSKNTLYLQMNS | | | | | | | |
| | AFLTIHNVAIE | | | | | | | | LRAEDTAVYYCA | | | | | | | |
| | DEAIYFCHSY | | | | | | | | KFRQYSGGFDY | | | | | | | |
| | VSSFNVFGGG | | | | | | | | WGQGTLVTVSS | | | | | | | |
| | TKLTVL | | | | | | | | | | | | | | | |
| CD3 | DIQMTQTTSSL | 697 | QDIR | 698 | YTS | 699 | QQG | 700 | EVQLQQSGPELV | 701 | GYSF | 702 | INPY | 703 | ARSG | 704 |
| | SASLGDRVTIS | | NY | | | | NTLP | | KPGASMKISCKA | | TGY | | KGVS | | YYGD | |
| | CRASQDIRNY | | | | | | WTF | | SGYSFTGYTMN | | T | | | | SDWY | |
| | LNWYQQKPD | | | | | | AGG | | WVKQSHGKNLE | | | | | | FDV | |
| | GTVKLLIYYTS | | | | | | | | WMGLINPYKGVS | | | | | | | |
| | RLHSGVPSKFS | | | | | | | | TYNQKFKDKATL | | | | | | | |
| | GSGSGTDYSL | | | | | | | | TVDKSSSTAYME | | | | | | | |
| | TISNLEQEDIA | | | | | | | | LLSLTSEDSAVY | | | | | | | |
| | TYFCQQGNTL | | | | | | | | YCARSGYYGDSD | | | | | | | |
| | PWTFAGGTKL | | | | | | | | WYFDVWGQGTT | | | | | | | |
| | EIK | | | | | | | | LTVFS | | | | | | | |
| CD3 | QTVVTQEPSL | 705 | TGAVT | 706 | GTK | 707 | VLW | 708 | EVQLVESGGGLV | 709 | GFTF | 710 | IRSK | 711 | VRHG | 712 |
| | TVSPGGTVTL | | SGNY | | | | YSNR | | QPGGSLKLSCAA | | NKY | | YNNY | | NFGN | |
| | TCGSSTGAVT | | | | | | WV | | SGFTFNKYAMN | | A | | AT | | SYIS | |
| | SGNYPNWVQ | | | | | | | | WVRQAPGKGLE | | | | | | YWAY | |
| | QKPGQAPRGL | | | | | | | | WVARIRSKYNNY | | | | | | | |
| | IGGTKFLAPGT | | | | | | | | ATYYADSVKDRF | | | | | | | |
| | PARFSGSLLGG | | | | | | | | TISRDDSKNTAY | | | | | | | |
| | KAALTLSGVQ | | | | | | | | LQMNNLKTEDT | | | | | | | |
| | PEDEAEYYCV | | | | | | | | AVYYCVRHGNF | | | | | | | |
| | LWYSNRWVF | | | | | | | | GNSYISYWAYW | | | | | | | |
| | GGGTKLTVL | | | | | | | | GQGTLVTVSS | | | | | | | |
| CD3 | DFVMTQSPDS | 713 | QSLL | 714 | WAS | 715 | QND | 716 | EVQLVQSGAELK | 717 | GYTF | 718 | IIPS | 719 | ARSH | 720 |
| | LAVSLGERVT | | NSGN | | | | YSYP | | KPGASVKVSCKA | | TDY | | NGAT | | LLRA | |
| | MSCKSSQSLL | | QKNY | | | | YT | | SGYTFTDYYMK | | Y | | | | SWFA | |
| | NSGNQKNYLT | | | | | | | | WVRQAPGQGLE | | | | | | YW | |
| | WYQQKPGQPP | | | | | | | | WIGDIIPSNGATF | | | | | | | |
| | KLLIYWASTR | | | | | | | | YNQKFKGRVTIT | | | | | | | |
| | ESGVPDRFSGS | | | | | | | | VDKSTSTAYMEL | | | | | | | |
| | GSGTDFTLTIS | | | | | | | | SSLRSEDTAVYY | | | | | | | |
| | SLQAEDVAVY | | | | | | | | CARSHLLRASWF | | | | | | | |
| | YCQNDYSYPY | | | | | | | | AYWGQGTLVTV | | | | | | | |
| | TFGQGTKLEIK | | | | | | | | SS | | | | | | | |
| CD3 | QIVLTQSPAIM | 721 | SSVS | 722 | DTS | 723 | QQW | 724 | QVQLQQSGAELA | 725 | GYTF | 726 | INPS | 727 | ARYY | 728 |
| | SASPGEKVTM | | Y | | | | SSNP | | RPGASVKMSCKA | | TRYT | | RGYT | | DDHY | |
| | TCSASSSVSY | | | | | | FT | | SGYTFTRYTMH | | | | | | CLDY | |
| | MNWYQQKSG | | | | | | | | WVKQRPGQGLE | | | | | | | |
| | TSPKRWIYDTS | | | | | | | | WIGYINPSRGYT | | | | | | | |
| | KLASGVPAHF | | | | | | | | NYNQKFKDKAT | | | | | | | |
| | RGSGSGTSYSL | | | | | | | | LTTDKSSSTAYM | | | | | | | |
| | TISGMEAEDA | | | | | | | | QLSSLTSEDSAV | | | | | | | |
| | ATYYCQQWSS | | | | | | | | YYCARYYDDHY | | | | | | | |
| | NPFTFGSGTKL | | | | | | | | CLDYWGQGTTL | | | | | | | |
| | EIN | | | | | | | | TVSS | | | | | | | |
| CD3 | EIVLTQSPATL | 729 | QSVS | 730 | DAS | 731 | QQRS | 732 | QVQLVESGGGV | 733 | GFKF | 734 | IWYD | 735 | ARQM | 736 |
| | SLSPGERATLS | | SY | | | | NWP | | VQPGRSLRLSCA | | SGY | | GSKK | | GYWH | |
| | CRASQSVSSY | | | | | | PLT | | ASGFKFSGYGMH | | G | | | | FDL | |
| | LAWYQQKPG | | | | | | | | WVRQAPGKGLE | | | | | | | |
| | QAPRLLIYDAS | | | | | | | | WVAVIWYDGSK | | | | | | | |
| | NRATGIPARFS | | | | | | | | KYYVDSVKGRFT | | | | | | | |
| | GSGSGTDFTLT | | | | | | | | ISRDNSKNTLYL | | | | | | | |
| | ISSLEPEDFAV | | | | | | | | QMNSLRAEDTA | | | | | | | |
| | YYCQQRSNWP | | | | | | | | VYYCARQMGYW | | | | | | | |
| | PLTFGGGTKV | | | | | | | | HFDLWGRGTLVT | | | | | | | |
| | EIK | | | | | | | | VSS | | | | | | | |
| CD3 | DIQMTQSPSSL | 737 | QSIS | 738 | AAS | 739 | QQS | 740 | QVQLVQSGAEV | 741 | GYTF | 742 | INPS | 743 | AKGT | 744 |
| | SASVGDRVTIT | | SY | | | | YSTP | | KKPGASVKVSCK | | TSYY | | GGST | | TGDW | |
| | CRASQSISSYL | | | | | | PT | | ASGYTFTSYYMH | | | | | | FDY | |
| | NWYQQKPGK | | | | | | | | WVRQAPGQGLE | | | | | | | |
| | APKLLIYAASS | | | | | | | | WMGIINPSGGSTS | | | | | | | |
| | LQSGVPSRFSG | | | | | | | | YAQKFQGRVTM | | | | | | | |
| | SGSGTDFTLTI | | | | | | | | TRDTSTSTVYME | | | | | | | |
| | SSLQPEDFATY | | | | | | | | LSSLRSEDTAVY | | | | | | | |
| | YCQQSYSTPPT | | | | | | | | YCAKGTTGDWF | | | | | | | |
| | FGQGTKVEIK | | | | | | | | DYWGQGTLVTV | | | | | | | |
| | | | | | | | | | SS | | | | | | | |
| CD30 | DIVLTQSPASL | 745 | QSVD | 746 | AAS | 747 | QQS | 748 | QIQLQQSGPEVV | 749 | GYTF | 750 | IYPG | 751 | ANYG | 752 |
| (TNFRS | AVSLGQRATIS | | FDGD | | | | NEDP | | KPGASVKISCKA | | TDY | | SGNT | | NYWF | |
| F8) | CKASQSVDFD | | SY | | | | WT | | SGYTFTDYYITW | | Y | | | | AY | |
| | GDSYMNWYQ | | | | | | | | VKQKPGQGLEWI | | | | | | | |
| | QKPGQPPKVLI | | | | | | | | GWIYPGSGNTKY | | | | | | | |
| | YAASNLESGIP | | | | | | | | NEKFKGKATLTV | | | | | | | |
| | ARFSGSGSGT | | | | | | | | DTSSSTAFMQLSS | | | | | | | |
| | DFTLNIHPVEE | | | | | | | | LTSEDTAVYFCA | | | | | | | |
| | EDAATYYCQQ | | | | | | | | NYGNYWFAYWG | | | | | | | |
| | SNEDPWTFGG | | | | | | | | QGTQVTVSA | | | | | | | |
| | GTKLEIK | | | | | | | | | | | | | | | |
| CD30 | DIQMTQSPTSL | 753 | QGIS | 754 | AAS | 755 | QQY | 756 | QVQLQQWGAGL | 757 | GGSF | 758 | INHG | 759 | ASLT | 760 |
| (TNFRS | SASVGDRVTIT | | SW | | | | DSYP | | LKPSETLSLTCAV | | SAY | | GGT | | AY | |
| F8) | CRASQGISSW | | | | | | IT | | YGGSFSAYYWS | | Y | | | | | |
| | LTWYQQKPEK | | | | | | | | WIRQPPGKGLEW | | | | | | | |
| | APKSLIYAASS | | | | | | | | IGDINHGGGTNY | | | | | | | |
| | LQSGVPSRFSG | | | | | | | | NPSLKSRVTISVD | | | | | | | |
| | SGSGTDFTLTI | | | | | | | | TSKNQFSLKLNS | | | | | | | |
| | SSLQPEDFATY | | | | | | | | VTAADTAVYYC | | | | | | | |
| | YCQQYDSYPI | | | | | | | | ASLTAYWGQGSL | | | | | | | |
| | TFGQGTRLEIK | | | | | | | | VTVSS | | | | | | | |
| CD319 | DIQMTQSPSSL | 761 | QDVG | 762 | WAS | 763 | QQY | 764 | EVQLVESGGGLV | 765 | GFDF | 766 | INPD | 767 | ARPD | 768 |
| (SLAMF | SASVGDRVTIT | | IA | | | | SSYP | | QPGGSLRLSCAA | | SRY | | SSTI | | GNYW | |
| 7) | CKASQDVGIA | | | | | | YT | | SGFDFSRYWMS | | W | | | | YFDV | |
| | VAWYQQKPG | | | | | | | | WVRQAPGKGLE | | | | | | | |
| | KVPKLLIYWA | | | | | | | | WIGEINPDSSTIN | | | | | | | |
| | STRHTGVPDR | | | | | | | | YAPSLKDKFIISR | | | | | | | |
| | FSGSGSGTDFT | | | | | | | | DNAKNSLYLQM | | | | | | | |
| | LTISSLQPEDV | | | | | | | | NSLRAEDTAVYY | | | | | | | |
| | ATYYCQQYSS | | | | | | | | CARPDGNYWYF | | | | | | | |
| | YPYTFGQGTK | | | | | | | | DVWGQGTLVTV | | | | | | | |
| | VEIK | | | | | | | | SS | | | | | | | |
| CD33 | DIVLTQSPTIM | 769 | SSVN | 770 | DTS | 771 | QQW | 772 | EVKLQESGPELV | 773 | GYK | 774 | INPY | 775 | ARDY | 776 |
| | SASPGERVTM | | Y | | | | RSYP | | KPGASVKMSCK | | FTDY | | NDGT | | RYEV | |
| | TCTASSSVNYI | | | | | | LT | | ASGYKFTDYVVH | | V | | | | YGMD | |
| | HWYQQKSGD | | | | | | | | WLKQKPGQGLE | | | | | | Y | |
| | SPLRWIFDTSK | | | | | | | | WIGYINPYNDGT | | | | | | | |
| | VASGVPARFS | | | | | | | | KYNEKFKGKATL | | | | | | | |
| | GSGSGTSYSLT | | | | | | | | TSDKSSSTAYME | | | | | | | |
| | ISTMEAEDAA | | | | | | | | VSSLTSEDSAVY | | | | | | | |
| | TYYCQQWRS | | | | | | | | YCARDYRYEVY | | | | | | | |
| | YPLTFGDGTR | | | | | | | | GMDYWGQGTSV | | | | | | | |
| | LELK | | | | | | | | TVSS | | | | | | | |
| CD33 | DIVMTQSPSSL | 777 | QDIN | 778 | YTS | 779 | LQY | 780 | EVKLQQSGPELV | 781 | GYSF | 782 | IDPY | 783 | AREM | 784 |
| | SASLGGKVTIT | | KY | | | | DNLL | | KPGTSVKVSCKA | | TDY | | KGGT | | ITAY | |
| | CKASQDINKYI | | | | | | T | | SGYSFTDYNMY | | N | | | | YFDY | |
| | AWYQHKPGK | | | | | | | | WVKQSHGKSLE | | | | | | | |
| | GPRLLIHYTST | | | | | | | | WIGYIDPYKGGTI | | | | | | | |
| | LQPGIPSRFSG | | | | | | | | YNQKFKGKATLT | | | | | | | |
| | SGSGRDYSFSI | | | | | | | | VDKSSSTAFMHL | | | | | | | |
| | SNLEPEDIATY | | | | | | | | NSLTSEDSAVYY | | | | | | | |
| | YCLQYDNLLT | | | | | | | | CAREMITAYYFD | | | | | | | |
| | FGAGTKLELK | | | | | | | | YWGQGSSVTVSS | | | | | | | |
| CD33 | DIVLTQSPASL | 785 | ESVD | 786 | AAS | 787 | QQS | 788 | EVQLQQSGPELV | 789 | GYTF | 790 | IYPY | 791 | ARGR | 792 |
| | AVSLGQRATIS | | NYGI | | | | KEVP | | KPGASVKISCKA | | TDY | | NGGT | | PAMD | |
| | CRASESVDNY | | SF | | | | WT | | SGYTFTDYNMH | | N | | | | Y | |
| | GISFMNWFQQ | | | | | | | | WVKQSHGKSLE | | | | | | | |
| | KPGQPPKLLIY | | | | | | | | WIGYIYPYNGGT | | | | | | | |
| | AASNQGSGVP | | | | | | | | GYNQKFKSKATL | | | | | | | |
| | ARFSGSGSGT | | | | | | | | TVDNSSSTAYMD | | | | | | | |
| | DFSLNIHPMEE | | | | | | | | VRSLTSEDSAVY | | | | | | | |
| | DDTAMYFCQ | | | | | | | | YCARGRPAMDY | | | | | | | |
| | QSKEVPWTFG | | | | | | | | WGQGTSVTVSS | | | | | | | |
| | GGTKLEIK | | | | | | | | | | | | | | | |
| CD33 | DIQLTQSPSTL | 793 | ESLD | 794 | AAS | 795 | QQT | 796 | EVQLVQSGAEVK | 797 | GYTI | 798 | IYPYN | 799 | VNGNP | 800 |
| | SASVGDRVTIT | | NYGI | | | | KEVP | | KPGSSVKVSCKA | | TDSN | | GGT | | WLAY | |
| | CRASESLDNY | | RF | | | | WS | | SGYTITDSNIHW | | | | | | | |
| | GIRFLTWFQQ | | | | | | | | VRQAPGQSLEWI | | | | | | | |
| | KPGKAPKLLM | | | | | | | | GYIYPYNGGTDY | | | | | | | |
| | YAASNQGSGV | | | | | | | | NQKFKNRATLTV | | | | | | | |
| | PSRFSGSGSGT | | | | | | | | DNPTNTAYMELS | | | | | | | |
| | EFTLTISSLQP | | | | | | | | SLRSEDTAFYYC | | | | | | | |
| | DDFATYYCQQ | | | | | | | | VNGNPWLAYWG | | | | | | | |
| | TKEVPWSFGQ | | | | | | | | QGTLVTVSS | | | | | | | |
| | GTKVEVK | | | | | | | | | | | | | | | |
| CD33 | NIMLTQSPSSL | 801 | QSVF | 802 | WAS | 803 | HQY | 804 | QVQLQQPGAEV | 805 | GYTF | 806 | IYPG | 807 | AREV | 808 |
| | AVSAGEKVTM | | FSSS | | | | LSSR | | VKPGASVKMSC | | TSYY | | NDDI | | RLRY | |
| | SCKSSQSVFFS | | QKNY | | | | T | | KASGYTFTSYYI | | | | | | FDV | |
| | SSQKNYLAWY | | | | | | | | HWIKQTPGQGLE | | | | | | | |
| | QQIPGQSPKLL | | | | | | | | WVGVIYPGNDDI | | | | | | | |
| | IYWASTRESG | | | | | | | | SYNQKFKGKATL | | | | | | | |
| | VPDRFTGSGS | | | | | | | | TADKSSTTAYMQ | | | | | | | |
| | GTDFTLTISSV | | | | | | | | LSSLTSEDSAVY | | | | | | | |
| | QSEDLAIYYC | | | | | | | | YCAREVRLRYFD | | | | | | | |
| | HQYLSSRTFG | | | | | | | | VWGAGTTVTVSS | | | | | | | |
| | GGTKLEIK | | | | | | | | | | | | | | | |
| CD33 | DIKMTQSPSS | 809 | QDIN | 810 | RAN | 811 | LQY | 812 | QVQLQQSGPELV | 813 | GYTF | 814 | IYPG | 815 | ASGY | 816 |
| | MYASLGERVII | | SY | | | | DEFP | | RPGTFVKISCKAS | | TNY | | DGST | | EDAM | |
| | NCKASQDINS | | | | | | LT | | GYTFTNYDINWV | | D | | | | DY | |
| | YLSWFQQKPG | | | | | | | | NQRPGQGLEWIG | | | | | | | |
| | KSPKTLIYRAN | | | | | | | | WIYPGDGSTKYN | | | | | | | |
| | RLVDGVPSRF | | | | | | | | EKFKAKATLTAD | | | | | | | |
| | SGSGSGQDYS | | | | | | | | KSSSTAYLQLNN | | | | | | | |
| | LTISSLEYEDM | | | | | | | | LTSENSAVYFCA | | | | | | | |
| | GIYYCLQYDE | | | | | | | | SGYEDAMDYWG | | | | | | | |
| | FPLTFGAGTKL | | | | | | | | QGTSVTVSS | | | | | | | |
| | ELK | | | | | | | | | | | | | | | |
| CD33 | DIQMTQSPSSL | 817 | ESVD | 818 | AAS | 819 | QQS | 820 | QVQLVQSGAEV | 821 | GYTF | 822 | IYPY | 823 | ARGR | 824 |
| | SASVGDRVTIT | | NYGI | | | | KEVP | | KKPGSSVKVSCK | | TDY | | NGGT | | PAMD | |
| | CRASESVDNY | | SF | | | | WT | | ASGYTFTDYNM | | N | | | | YW | |
| | GISFMNWFQQ | | | | | | | | HWVRQAPGQGL | | | | | | | |
| | KPGKAPKLLL | | | | | | | | EWLGYIYPYNGG | | | | | | | |
| | YAASNQGSGV | | | | | | | | TGYNQKFKSKAT | | | | | | | |
| | PSRFSGSGSGT | | | | | | | | ITADESTNTAYM | | | | | | | |
| | DFTLTISSLQP | | | | | | | | ELSSLRSEDTAV | | | | | | | |
| | DDFATYYCQQ | | | | | | | | YYCARGRPAMD | | | | | | | |
| | SKEVPWTFGQ | | | | | | | | YWGQGTLVTVSS | | | | | | | |
| | GTKVELK | | | | | | | | | | | | | | | |
| CD332 | QSVLTQPPSAS | 825 | SSNI | 826 | ENY | 827 | SSW | 828 | EVQLLESGGGLV | 829 | GFTF | 830 | ISGS | 831 | ARVR | 832 |
| (FGFR2) | GTPGQRVTISC | | GNNY | | N | | DDSL | | QPGGSLRLSCAA | | SSYA | | GTST | | YNWN | |
| | SGSSSNIGNNY | | | | | | NYW | | SGFTFSSYAMSW | | | | | | HGDW | |
| | VSWYQQLPGT | | | | | | V | | VRQAPGKGLEW | | | | | | FDP | |
| | APKLLIYENY | | | | | | | | VSAISGSGTSTYY | | | | | | | |
| | NRPAGVPDRF | | | | | | | | ADSVKGRFTISR | | | | | | | |
| | SGSKSGTSASL | | | | | | | | DNSKNTLYLQM | | | | | | | |
| | AISGLRSEDEA | | | | | | | | NSLRAEDTAVYY | | | | | | | |
| | DYYCSSWDDS | | | | | | | | CARVRYNWNHG | | | | | | | |
| | LNYWVFGGG | | | | | | | | DWFDPWGQGTL | | | | | | | |
| | TKLTVL | | | | | | | | VTVSS | | | | | | | |
| CD350 | DIQMTQSPASL | 833 | ENIY | 834 | VAT | 835 | QHF | 836 | EVQLQQSGAELV | 837 | GFNI | 838 | IDPA | 839 | ARGA | 840 |
| (FZD | SVSVGETVTIT | | SN | | | | WGT | | KPGASVKLSCTA | | NDT | | NGNT | | RGSR | |
| 10) | CRASENIYSNL | | | | | | PYT | | SGFNINDTYMHW | | Y | | | | FAY | |
| | AWYQQKQGK | | | | | | | | VKQRPEQGLEWI | | | | | | | |
| | SPQLLVYVAT | | | | | | | | GRIDPANGNTKY | | | | | | | |
| | NLADGVPSRF | | | | | | | | DPKFQGKATITA | | | | | | | |
| | SGSGSGTQYS | | | | | | | | DTSSNTAYLQLS | | | | | | | |
| | LKINSLQSEDF | | | | | | | | SLTSEDTAVYYC | | | | | | | |
| | GSYYCQHFW | | | | | | | | ARGARGSRFAY | | | | | | | |
| | GTPYTFGGGT | | | | | | | | WGQGTLVTVSA | | | | | | | |
| | KLEIK | | | | | | | | | | | | | | | |
| CD37 | DIQMTQSPSSL | 841 | ENIR | 842 | VAT | 843 | QHY | 844 | QVQVQESGPGLV | 845 | GFSL | 846 | IWGD | 847 | AKGG | 848 |
| | SVSVGERVTIT | | SN | | | | WGT | | APSQTLSITCTVS | | TTSG | | GST | | YSLA | |
| | CRASENIRSNL | | | | | | TWT | | GFSLTTSGVSWV | | | | | | H | |
| | AWYQQKPGK | | | | | | | | RQPPGKGLEWLG | | | | | | | |
| | SPKLLVNVAT | | | | | | | | VIWGDGSTNYHP | | | | | | | |
| | NLADGVPSRF | | | | | | | | SLKSRLSIKKDHS | | | | | | | |
| | SGSGSGTDYS | | | | | | | | KSQVFLKLNSLT | | | | | | | |
| | LKINSLQPEDF | | | | | | | | AADTATYYCAK | | | | | | | |
| | GTYYCQHYW | | | | | | | | GGYSLAHWGQG | | | | | | | |
| | GTTWTFGQGT | | | | | | | | TLVTVSS | | | | | | | |
| | KLEIK | | | | | | | | | | | | | | | |
| CD371 | DIQMTQSPSSL | 849 | QSIS | 850 | AAS | 851 | QQS | 852 | QVQLVQSGGGV | 853 | GFTF | 854 | IWYN | 855 | TRGT | 856 |
| (CLEC1 | SASVGDRVTIT | | SY | | | | YSTP | | VQPGRSLRLSCV | | SSYG | | ARKQ | | GYNW | |
| 2A) | CRASQSISSYL | | | | | | PT | | ASGFTFSSYGMH | | | | | | FDP | |
| | NWYQQKPGK | | | | | | | | WVRQAPGKGLE | | | | | | | |
| | APKLLIYAASS | | | | | | | | WVAAIWYNARK | | | | | | | |
| | LQSGVPSRFSG | | | | | | | | QDYADSVKGRFT | | | | | | | |
| | SGSGTDFTLTI | | | | | | | | ISRDNSKNTLYL | | | | | | | |
| | SSLQPEDFATY | | | | | | | | QMNSLRAEDTA | | | | | | | |
| | YCQQSYSTPPT | | | | | | | | VYYCTRGTGYN | | | | | | | |
| | FGQGTKVEIK | | | | | | | | WFDPWGQGTLV | | | | | | | |
| | | | | | | | | | TVSS | | | | | | | |
| CD38 | DIVMAQSHKF | 857 | ASQD | 858 | ITSA | 859 | TCQ | 860 | QVKLVESGGGLV | 861 | GFTF | 862 | ISIG | 863 | TRDF | 864 |
| | MSTSVGDRVS | | VS | | | | QHY | | KPGGSLKLSCEA | | SSYT | | GRYT | | NGTS | |
| | ITCKASQDVST | | | | | | SPYT | | SGFTFSSYTLSW | | | | | | DF | |
| | VVAWYQQKP | | | | | | | | VRQTPETRLEWV | | | | | | | |
| | GQSPKRLITSA | | | | | | | | ATISIGGRYTTTP | | | | | | | |
| | SYRYIGVPDRF | | | | | | | | DSVEGRFTISRDN | | | | | | | |
| | TGSGSGTDFTF | | | | | | | | AKNTLYLQMNSL | | | | | | | |
| | TISSVQAEDLA | | | | | | | | KSEDTAMYYCTR | | | | | | | |
| | VTTCQQHYSP | | | | | | | | DFNGTSDFWGQ | | | | | | | |
| | YTFGGGTKLEI | | | | | | | | GTTLTVSS | | | | | | | |
| | K | | | | | | | | | | | | | | | |
| CD38 | EIVLTQSPATL | 865 | QSVS | 866 | DAS | 867 | QQRS | 868 | EVQLLESGGGLV | 869 | GFTF | 870 | ISGS | 871 | AKDK | 872 |
| | SLSPGERATLS | | SY | | | | NWP | | QPGGSLRLSCAV | | NSFA | | GGGT | | ILWF | |
| | CRASQSVSSY | | | | | | PT | | SGFTFNSFAMSW | | | | | | GEPV | |
| | LAWYQQKPG | | | | | | | | VRQAPGKGLEW | | | | | | FDY | |
| | QAPRLLIYDAS | | | | | | | | VSAISGSGGGTY | | | | | | | |
| | NRATGIPARFS | | | | | | | | YADSVKGRFTIS | | | | | | | |
| | GSGSGTDFTLT | | | | | | | | RDNSKNTLYLQ | | | | | | | |
| | ISSLEPEDFAV | | | | | | | | MNSLRAEDTAV | | | | | | | |
| | YYCQQRSNWP | | | | | | | | YFCAKDKILWFG | | | | | | | |
| | PTFGQGTKVEI | | | | | | | | EPVFDYWGQGTL | | | | | | | |
| | K | | | | | | | | VTVSS | | | | | | | |
| CD38 | DIVMTQSHLS | 873 | QDVS | 874 | SAS | 875 | QQH | 876 | QVQLVQSGAEV | 877 | GYTF | 878 | IYPG | 879 | ARGDY | 880 |
| | MSTSLGDPVSI | | TV | | | | YSPP | | AKPGTSVKLSCK | | TDY | | GDT | | DYGS | |
| | TCKASQDVST | | | | | | YT | | ASGYTFTDYWM | | W | | | | NSLD | |
| | VVAWYQQKP | | | | | | | | QWVKQRPGQGL | | | | | | Y | |
| | GQSPRRLIYSA | | | | | | | | EWIGTIYPGDGD | | | | | | | |
| | SYRYIGVPDRF | | | | | | | | TGYAQKFQGKA | | | | | | | |
| | TGSGAGTDFT | | | | | | | | TLTADKSSKTVY | | | | | | | |
| | FTISSVQAEDL | | | | | | | | MHLSSLASEDSA | | | | | | | |
| | AVYYCQQHYS | | | | | | | | VYYCARGDYYG | | | | | | | |
| | PPYTFGGGTK | | | | | | | | SNSLDYWGQGTS | | | | | | | |
| | LEIK | | | | | | | | VTVSS | | | | | | | |
| CD4 | DIVMTQSPDSL | 881 | KSVS | 882 | LAS | 883 | QHSR | 884 | EEQLVESGGGLV | 885 | GFSF | 886 | ISVK | 887 | SASY | 888 |
| | AVSLGERATIN | | TSGY | | | | ELP | | KPGGSLRLSCAA | | SDCR | | SENY | | YRYD | |
| | CRASKSVSTS | | SY | | | | WT | | SGFSFSDCRMYW | | | | GA | | VGAW | |
| | GYSYIYWYQQ | | | | | | | | LRQAPGKGLEWI | | | | | | FAYW | |
| | KPGQPPKLLIY | | | | | | | | GVISVKSENYGA | | | | | | | |
| | LASILESGVPD | | | | | | | | NYAESVRGRFTIS | | | | | | | |
| | RFSGSGSGTDF | | | | | | | | RDDSKNTVYLQ | | | | | | | |
| | TLTISSLQAED | | | | | | | | MNSLKTEDTAVY | | | | | | | |
| | VAVYYCQHSR | | | | | | | | YCSASYYRYDVG | | | | | | | |
| | ELPWTFGQGT | | | | | | | | AWFAYWGQGTL | | | | | | | |
| | KVEIK | | | | | | | | VTVSS | | | | | | | |
| CD4 | DIVMTQSPDSL | 889 | QSLL | 890 | WAS | 891 | QQY | 892 | QVQLQQSGPEVV | 893 | GYTF | 894 | INPY | 895 | AREK | 896 |
| | AVSLGERVTM | | YSTN | | | | YSYR | | KPGASVKMSCK | | TSYV | | NDGT | | DNYA | |
| | NCKSSQSLLYS | | QKNY | | | | T | | ASGYTFTSYVIH | | | | | | TGAW | |
| | TNQKNYLAW | | | | | | | | WVRQKPGQGLD | | | | | | FAY | |
| | YQQKPGQSPK | | | | | | | | WIGYINPYNDGT | | | | | | | |
| | LLIYWASTRES | | | | | | | | DYDEKFKGKATL | | | | | | | |
| | GVPDRFSGSG | | | | | | | | TSDTSTSTAYME | | | | | | | |
| | SGTDFTLTISS | | | | | | | | LSSLRSEDTAVY | | | | | | | |
| | VQAEDVAVY | | | | | | | | YCAREKDNYAT | | | | | | | |
| | YCQQYYSYRT | | | | | | | | GAWFAYWGQGT | | | | | | | |
| | FGGGTKLEIK | | | | | | | | LVTVSS | | | | | | | |
| CD40 | DIQMTQSPSSL | 897 | QSLV | 898 | TVS | 899 | SQTT | 900 | EVQLVESGGGLV | 901 | GYSF | 902 | VIPN | 903 | AREG | 904 |
| | SASVGDRVTIT | | HSNG | | | | HVP | | QPGGSLRLSCAA | | TGY | | AGGT | | IYW | |
| | CRSSQSLVHS | | NTF | | | | WT | | SGYSFTGYYIHW | | Y | | | | | |
| | NGNTFLHWY | | | | | | | | VRQAPGKGLEW | | | | | | | |
| | QQKPGKAPKL | | | | | | | | VARVIPNAGGTS | | | | | | | |
| | LIYTVSNRFSG | | | | | | | | YNQKFKGRFTLS | | | | | | | |
| | VPSRFSGSGSG | | | | | | | | VDNSKNTAYLQ | | | | | | | |
| | TDFTLTISSLQ | | | | | | | | MNSLRAEDTAV | | | | | | | |
| | PEDFATYFCSQ | | | | | | | | YYCAREGIYWW | | | | | | | |
| | TTHVPWTFGQ | | | | | | | | GQGTLVTVSS | | | | | | | |
| | GTKVEIK | | | | | | | | | | | | | | | |
| CD40 | AIQLTQSPSSL | 905 | QGIS | 906 | DAS | 907 | QQF | 908 | QLQLQESGPGLL | 909 | GGSI | 910 | IYKS | 911 | TRPV | 912 |
| | SASVGDRVTIT | | SA | | | | NSYP | | KPSETLSLTCTVS | | SSPG | | GST | | VRYF | |
| | CRASQGISSAL | | | | | | T | | GGSISSPGYYGG | | YY | | | | GWFD | |
| | AWYQQKPGK | | | | | | | | WIRQPPGKGLEW | | | | | | P | |
| | APKLLIYDASN | | | | | | | | IGSIYKSGSTYHN | | | | | | | |
| | LESGVPSRFSG | | | | | | | | PSLKSRVTISVDT | | | | | | | |
| | SGSGTDFTLTI | | | | | | | | SKNQFSLKLSSVT | | | | | | | |
| | SSLQPEDFATY | | | | | | | | AADTAVYYCTRP | | | | | | | |
| | YCQQFNSYPT | | | | | | | | VVRYFGWFDPW | | | | | | | |
| | FGQGTKVEIK | | | | | | | | GQGTLVTVSS | | | | | | | |
| CD40 | DIVMTQSPLSL | 913 | QSLL | 914 | LGS | 915 | MQA | 916 | QVQLVESGGGV | 917 | GFTF | 918 | ISYE | 919 | ARDG | 920 |
| | TVTPGEPASIS | | YSNG | | | | RQTP | | VQPGRSLRLSCA | | SSYG | | ESNR | | GIAA | |
| | CRSSQSLLYSN | | YNY | | | | FT | | ASGFTFSSYGMH | | | | | | PGPD | |
| | GYNYLDWYL | | | | | | | | WVRQAPGKGLE | | | | | | Y | |
| | QKPGQSPQVLI | | | | | | | | WVAVISYEESNR | | | | | | | |
| | SLGSNRASGV | | | | | | | | YHADSVKGRFTI | | | | | | | |
| | PDRFSGSGSGT | | | | | | | | SRDNSKITLYLQ | | | | | | | |
| | DFTLKISRVEA | | | | | | | | MNSLRTEDTAVY | | | | | | | |
| | EDVGVYYCM | | | | | | | | YCARDGGIAAPG | | | | | | | |
| | QARQTPFTFGP | | | | | | | | PDYWGQGTLVT | | | | | | | |
| | GTKVDIR | | | | | | | | VSS | | | | | | | |
| CD40 | DIQMTQSPSSV | 921 | QGIY | 922 | TAS | 923 | QQA | 924 | QVQLVQSGAEV | 925 | GYTF | 926 | INPD | 927 | ARDQ | 928 |
| | SASVGDRVTIT | | SW | | | | NIFP | | KKPGASVKVSCK | | TGY | | SGGT | | PLGY | |
| | CRASQGIYSW | | | | | | LT | | ASGYTFTGYYM | | Y | | | | CTNG | |
| | LAWYQQKPG | | | | | | | | HWVRQAPGQGL | | | | | | VCSY | |
| | KAPNLLIYTAS | | | | | | | | EWMGWINPDSG | | | | | | FDY | |
| | TLQSGVPSRFS | | | | | | | | GTNYAQKFQGR | | | | | | | |
| | GSGSGTDFTLT | | | | | | | | VTMTRDTSISTA | | | | | | | |
| | ISSLQPEDFAT | | | | | | | | YMELNRLRSDDT | | | | | | | |
| | YYCQQANIFP | | | | | | | | AVYYCARDQPL | | | | | | | |
| | LTFGGGTKVEI | | | | | | | | GYCTNGVCSYFD | | | | | | | |
| | K | | | | | | | | YWGQGTLVTVSS | | | | | | | |
| CD40L | DIQMTQSPSSL | 929 | EDLY | 930 | DTY | 931 | QQY | 932 | EVQLVESGGGLV | 933 | GFSS | 934 | IWGD | 935 | ARQL | 936 |
| | SASVGDRVTIT | | YN | | | | YKFP | | QPGGSLRLSCAV | | TNY | | GDT | | THYY | |
| | CRASEDLYYN | | | | | | FT | | SGFSSTNYHVHW | | H | | | | VLAA | |
| | LAWYQRKPG | | | | | | | | VRQAPGKGLEW | | | | | | | |
| | KAPKLLIYDT | | | | | | | | MGVIWGDGDTS | | | | | | | |
| | YRLADGVPSR | | | | | | | | YNSVLKSRFTISR | | | | | | | |
| | FSGSGSGTDY | | | | | | | | DTSKNTVYLQM | | | | | | | |
| | TLTISSLQPED | | | | | | | | NSLRAEDTAVYY | | | | | | | |
| | FASYYCQQYY | | | | | | | | CARQLTHYYVLA | | | | | | | |
| | KFPFTFGQGT | | | | | | | | AWGQGTLVTVSS | | | | | | | |
| | KVEIK | | | | | | | | | | | | | | | |
| CD40L | DIVLTQSPATL | 937 | QRVS | 938 | YAS | 939 | QHS | 940 | QVQLVQSGAEV | 941 | GYIF | 942 | INPS | 943 | TRSD | 944 |
| | SVSPGERATIS | | SSTY | | | | WEIP | | VKPGASVKLSCK | | TSYY | | NGDT | | GRND | |
| | CRASQRVSSST | | SY | | | | PT | | ASGYIFTSYYMY | | | | | | MDS | |
| | YSYMHWYQQ | | | | | | | | WVKQAPGQGLE | | | | | | | |
| | KPGQPPKLLIK | | | | | | | | WIGEINPSNGDT | | | | | | | |
| | YASNLESGVP | | | | | | | | NFNEKFKSKATL | | | | | | | |
| | ARFSGSGSGT | | | | | | | | TVDKSASTAYME | | | | | | | |
| | DFTLTISSVEP | | | | | | | | LSSLRSEDTAVY | | | | | | | |
| | EDFATYYCQH | | | | | | | | YCTRSDGRNDM | | | | | | | |
| | SWEIPPTFGGG | | | | | | | | DSWGQGTLVTVS | | | | | | | |
| | TKLEIK | | | | | | | | S | | | | | | | |
| | DFVMTQSPAF | | | | | | | | | | | | | | | |
| CD49b | LSVTPGEKVTI | 945 | SSVN | 946 | DTS | 947 | QQW | 948 | QVQLQESGPGLV | 949 | GFSL | 950 | IWAR | 951 | ARAN | 952 |
| (a2) | TCSAQSSVNYI | | Y | | | | TTNP | | KPSETLSLTCTVS | | TNY | | GFT | | DGVY | |
| | HWYQQKPDQ | | | | | | LT | | GFSLTNYGIHWIR | | G | | | | YAMD | |
| | APKKLIYDTSK | | | | | | | | QPPGKGLEWLGV | | | | | | Y | |
| | LASGVPSRFSG | | | | | | | | IWARGFTNYNSA | | | | | | | |
| | SGSGTDYTFTI | | | | | | | | LMSRLTISKDNS | | | | | | | |
| | SSLEAEDAAT | | | | | | | | KNQVSLKLSSVT | | | | | | | |
| | YYCQQWTTNP | | | | | | | | AADTAVYYCAR | | | | | | | |
| | LTFGQGTKVEI | | | | | | | | ANDGVYYAMDY | | | | | | | |
| | K | | | | | | | | WGQGTLVTVSS | | | | | | | |
| CD51 | DIQMTQSPSSL | 953 | QDIS | 954 | YTS | 955 | QQG | 956 | QVQLQQSGGELA | 957 | GYTF | 958 | INPRS | 959 | ASFL | 960 |
| (a5) | SASVGDRVTIT | | NY | | | | NTFP | | KPGASVKVSCKA | | SSFW | | GYT | | GRGA | |
| | CRASQDISNYL | | | | | | YT | | SGYTFSSFWMH | | | | | | MDY | |
| | AWYQQKPGK | | | | | | | | WVRQAPGQGLE | | | | | | | |
| | APKLLIYYTSK | | | | | | | | WIGYINPRSGYTE | | | | | | | |
| | IHSGVPSRFSG | | | | | | | | YNEIFRDKATMT | | | | | | | |
| | SGSGTDYTFTI | | | | | | | | TDTSTSTAYMEL | | | | | | | |
| | SSLQPEDIATY | | | | | | | | SSLRSEDTAVYY | | | | | | | |
| | YCQQGNTFPY | | | | | | | | CASFLGRGAMD | | | | | | | |
| | TFGQGTKVEI | | | | | | | | YWGQGTTVTVSS | | | | | | | |
| | K | | | | | | | | | | | | | | | |
| CD52 | DIQMTQSPSSL | 961 | QNID | 962 | NTN | 963 | LQHI | 964 | QVQLQESGPGLV | 965 | GFTF | 966 | IRDK | 967 | AREG | 968 |
| | SASVGDRVTIT | | KY | | | | SRPR | | RPSQTLSLTCTVS | | TDFY | | AKGY | | HTAA | |
| | CKASQNIDKY | | | | | | T | | GFTFTDFYMNW | | | | TT | | PFDY | |
| | LNWYQQKPG | | | | | | | | VRQPPGRGLEWI | | | | | | | |
| | KAPKLLIYNT | | | | | | | | GFIRDKAKGYTT | | | | | | | |
| | NNLQTGVPSR | | | | | | | | EYNPSVKGRVTM | | | | | | | |
| | FSGSGSGTDFT | | | | | | | | LVDTSKNQFSLR | | | | | | | |
| | FTISSLQPEDIA | | | | | | | | LSSVTAADTAVY | | | | | | | |
| | TYYCLQHISRP | | | | | | | | YCAREGHTAAPF | | | | | | | |
| | RTFGQGTKVEI | | | | | | | | DYWGQGSLVTV | | | | | | | |
| | K | | | | | | | | SS | | | | | | | |
| CD54(IC | QSVLTQPPSAS | 969 | SSNI | 970 | DNN | 971 | QSY | 972 | EVQLLESGGGLV | 973 | GFTF | 974 | IWYD | 975 | ARYS | 976 |
| AM-1) | GTPGQRVTISC | | GAGY | | | | DSSL | | QPGGSLRLSCAA | | SNA | | GSNK | | GWYF | |
| | TGSSSNIGAGY | | D | | | | SAW | | SGFTFSNAWMS | | W | | | | Y | |
| | DVHWYQQLP | | | | | | L | | WVRQAPGKGLE | | | | | | | |
| | GTAPKLLIYD | | | | | | | | WVAFIWYDGSN | | | | | | | |
| | NNNRPSGVPD | | | | | | | | KYYADSVKGRFT | | | | | | | |
| | RFSGSKSGTSA | | | | | | | | ISRDNSKNTLYL | | | | | | | |
| | SLAISGLRSED | | | | | | | | QMNSLRAEDTA | | | | | | | |
| | EADYYCQSYD | | | | | | | | VYYCARYSGWY | | | | | | | |
| | SSLSAWLFGG | | | | | | | | FDYWGQGTLVT | | | | | | | |
| | GTKLTVL | | | | | | | | VSS | | | | | | | |
| CD56 | DVVMTQSPLS | 977 | QIII | 978 | KVS | 979 | FQGS | 980 | QVQLVESGGGV | 981 | GFTF | 982 | ISSG | 983 | ARMR | 984 |
| | LPVTLGQPASI | | HSDG | | | | HVP | | VQPGRSLRLSCA | | SSFG | | SFTI | | KGYA | |
| | SCRSSQIIIHSD | | NTY | | | | HT | | ASGFTFSSFGMH | | | | | | MDY | |
| | GNTYLEWFQQ | | | | | | | | WVRQAPGKGLE | | | | | | | |
| | RPGQSPRRLIY | | | | | | | | WVAYISSGSFTIY | | | | | | | |
| | KVSNRFSGVP | | | | | | | | YADSVKGRFTIS | | | | | | | |
| | DRFSGSGSGT | | | | | | | | RDNSKNTLYLQ | | | | | | | |
| | DFTLKISRVEA | | | | | | | | MNSLRAEDTAV | | | | | | | |
| | EDVGVYYCFQ | | | | | | | | YYCARMRKGYA | | | | | | | |
| | GSHVPHTFGQ | | | | | | | | MDYWGQGTLVT | | | | | | | |
| | GTKVEIK | | | | | | | | VSS | | | | | | | |
| CD61 | EIVLTQSPATL | 985 | QSIS | 986 | YRS | 987 | QQS | 988 | QVQLVESGGGV | 989 | GFTF | 990 | VSSG | 991 | ARHL | 992 |
| (a4b3) | SLSPGERATLS | | NF | | | | GSW | | VQPGRSLRLSCA | | SSYD | | GGST | | HGSF | |
| | CQASQSISNFL | | | | | | PLT | | ASGFTFSSYDMS | | | | | | AS | |
| | HWYQQRPGQ | | | | | | | | WVRQAPGKGLE | | | | | | | |
| | APRLLIRYRSQ | | | | | | | | WVAKVSSGGGS | | | | | | | |
| | SISGIPARFSGS | | | | | | | | TYYLDTVQGRFT | | | | | | | |
| | GSGTDFTLTIS | | | | | | | | ISRDNSKNTLYL | | | | | | | |
| | SLEPEDFAVY | | | | | | | | QMNSLRAEDTA | | | | | | | |
| | YCQQSGSWPL | | | | | | | | VYYCARHLHGSF | | | | | | | |
| | TFGGGTKVEI | | | | | | | | ASWGQGTTVTVS | | | | | | | |
| | K | | | | | | | | S | | | | | | | |
| CD70 | QAVVTQEPSL | 993 | SGSV | 994 | NTN | 995 | ALFI | 996 | EVQLVESGGGLV | 997 | GFTF | 998 | INNE | 999 | ARDA | 1000 |
| | TVSPGGTVTL | | TSDN | | | | SNPS | | QPGGSLRLSCAA | | SVY | | GGTT | | GYSN | |
| | TCGLKSGSVT | | F | | | | VEFG | | SGFTFSVYYMN | | Y | | | | HVPI | |
| | SDNFPTWYQQ | | | | | | G | | WVRQAPGKGLE | | | | | | FDS | |
| | TPGQAPRLLIY | | | | | | | | WVSDINNEGGTT | | | | | | | |
| | NTNTRHSGVP | | | | | | | | YYADSVKGRFTI | | | | | | | |
| | DRFSGSILGNK | | | | | | | | SRDNSKNSLYLQ | | | | | | | |
| | AALTITGAQA | | | | | | | | MNSLRAEDTAV | | | | | | | |
| | DDEAEYFCAL | | | | | | | | YYCARDAGYSN | | | | | | | |
| | FISNPSVEFGG | | | | | | | | HVPIFDSWGQGT | | | | | | | |
| | GTQLTVL | | | | | | | | LVTVSS | | | | | | | |
| CD73 | QSVLTQPPSAS | 1001 | LSNI | 1002 | LDN | 1003 | ATW | 1004 | EVQLLESGGGLV | 1005 | GFTF | 1006 | ISGS | 1007 | ARLG | 1008 |
| (NT5E) | GTPGQRVTISC | | GRNP | | | | DDS | | QPGGSLRLSCAA | | SSYA | | GGRT | | YGRV | |
| | SGSLSNIGRNP | | | | | | HPG | | SGFTFSSYAYSW | | | | | | DE | |
| | VNWYQQLPG | | | | | | WT | | VRQAPGKGLEW | | | | | | | |
| | TAPKLLIYLDN | | | | | | | | VSAISGSGGRTY | | | | | | | |
| | LRLSGVPDRFS | | | | | | | | YADSVKGRFTIS | | | | | | | |
| | GSKSGTSASL | | | | | | | | RDNSKNTLYLQ | | | | | | | |
| | AISGLQSEDEA | | | | | | | | MNSLRAEDTAV | | | | | | | |
| | DYYCATWDD | | | | | | | | YYCARLGYGRV | | | | | | | |
| | SHPGWTFGGG | | | | | | | | DEWGRGTLVTVS | | | | | | | |
| | TKLTVL | | | | | | | | S | | | | | | | |
| CD74 | DIQLTQSPLSL | 1009 | QSLV | 1010 | TVS | 1011 | SQSS | 1012 | QVQLQQSGSELK | 1013 | GYTF | 1014 | INPN | 1015 | SRSR | 1016 |
| | PVTLGQPASIS | | HRNG | | | | HVPP | | KPGASVKVSCKA | | TNY | | TGEP | | GKNE | |
| | CRSSQSLVHR | | NTY | | | | T | | SGYTFTNYGVN | | G | | | | AWFA | |
| | NGNTYLHWF | | | | | | | | WIKQAPGQGLQ | | | | | | Y | |
| | QQRPGQSPRL | | | | | | | | WMGWINPNTGE | | | | | | | |
| | LIYTVSNRFSG | | | | | | | | PTFDDDFKGRFA | | | | | | | |
| | VPDRFSGSGS | | | | | | | | FSLDTSVSTAYL | | | | | | | |
| | GTDFTLKISRV | | | | | | | | QISSLKADDTAV | | | | | | | |
| | EAEDVGVYFC | | | | | | | | YFCSRSRGKNEA | | | | | | | |
| | SQSSHVPPTFG | | | | | | | | WFAYWGQGTLV | | | | | | | |
| | AGTRLEIK | | | | | | | | TVSS | | | | | | | |
| CEA | QTVLSQSPAIL | 1017 | SSVT | 1018 | ATS | 1019 | QHW | 1020 | EVKLVESGGGLV | 1021 | GFTF | 1022 | IGNK | 1023 | TRDR | 1024 |
| | SASPGEKVTM | | Y | | | | SSKP | | QPGGSLRLSCAT | | TDY | | ANGY | | GLRF | |
| | TCRASSSVTYI | | | | | | PT | | SGFTFTDYYMN | | Y | | TT | | YFDY | |
| | HWYQQKPGSS | | | | | | | | WVRQPPGKALE | | | | | | | |
| | PKSWIYATSN | | | | | | | | WLGFIGNKANGY | | | | | | | |
| | LASGVPARFS | | | | | | | | TTEYSASVKGRF | | | | | | | |
| | GSGSGTSYSLT | | | | | | | | TISRDKSQSILYL | | | | | | | |
| | ISRVEAEDAAT | | | | | | | | QMNTLRAEDSAT | | | | | | | |
| | YYCQHWSSKP | | | | | | | | YYCTRDRGLRFY | | | | | | | |
| | PTFGGGTKLEI | | | | | | | | FDYWGQGTTLT | | | | | | | |
| | K | | | | | | | | VSS | | | | | | | |
| CEA | DIQLTQSPSSL | 1025 | QDVG | 1026 | WTS | 1027 | QQY | 1028 | EVQLVESGGGVV | 1029 | GFDF | 1030 | IHPD | 1031 | ASLY | 1032 |
| | SASVGDRVTIT | | TS | | | | SLYR | | QPGRSLRLSCSAS | | TTY | | SSTI | | FGFP | |
| | CKASQDVGTS | | | | | | S | | GFDFTTYWMSW | | W | | | | WFAY | |
| | VAWYQQKPG | | | | | | | | VRQAPGKGLEWI | | | | | | | |
| | KAPKLLIYWT | | | | | | | | GEIHPDSSTINYA | | | | | | | |
| | STRHTGVPSRF | | | | | | | | PSLKDRFTISRDN | | | | | | | |
| | SGSGSGTDFTF | | | | | | | | AKNTLFLQMDSL | | | | | | | |
| | TISSLQPEDIAT | | | | | | | | RPEDTGVYFCAS | | | | | | | |
| | YYCQQYSLYR | | | | | | | | LYFGFPWFAYW | | | | | | | |
| | SFGQGTKVEIK | | | | | | | | GQGTPVTVSS | | | | | | | |
| Clau- | DIVMTQSPSSL | 1033 | QSLL | 1034 | WAS | 1035 | QND | 1036 | QVQLQQPGAELV | 1037 | GYTF | 1038 | IYPS | 1039 | TRSW | 1040 |
| din- | TVTAGEKVTM | | NSGN | | | | YSYP | | RPGASVKLSCKA | | TSY | | DSYT | | RGNS | |
| 18.2 | SCKSSQSLLNS | | QKNY | | | | FT | | SGYTFTSYWINW | | | | | | | |
| | GNQKNYLTW | | | | | | | | VKQRPGQGLEWI | | W | | | | FDY | |
| | YQQKPGQPPK | | | | | | | | GNIYPSDSYTNY | | | | | | | |
| | LLIYWASTRES | | | | | | | | NQKFKDKATLTV | | | | | | | |
| | GVPDRFTGSG | | | | | | | | DKSSSTAYMQLS | | | | | | | |
| | SGTDFTLTISS | | | | | | | | SPTSEDSAVYYC | | | | | | | |
| | VQAEDLAVYY | | | | | | | | TRSWRGNSFDY | | | | | | | |
| | CQNDYSYPFT | | | | | | | | WGQGTTLTVSS | | | | | | | |
| | FGSGTKLEIK | | | | | | | | | | | | | | | |
| cMET | DIQMTQSPSSL | 1041 | QSLL | 1042 | WAS | 1043 | QQY | 1044 | EVQLVESGGGLV | 1045 | GYTF | 1046 | IDPS | 1047 | ATYR | 1048 |
| | SASVGDRVTIT | | YTSS | | | | YAY | | QPGGSLRLSCAA | | TSY | | NSDT | | SYVT | |
| | CKSSQSLLYTS | | QKNY | | | | PWT | | SGYTFTSYWLH | | W | | | | PLDY | |
| | SQKNYLAWY | | | | | | | | WVRQAPGKGLE | | | | | | | |
| | QQKPGKAPKL | | | | | | | | WVGMIDPSNSDT | | | | | | | |
| | LIYWASTRES | | | | | | | | RFNPNFKDRFTIS | | | | | | | |
| | GVPSRFSGSGS | | | | | | | | ADTSKNTAYLQ | | | | | | | |
| | GTDFTLTISSL | | | | | | | | MNSLRAEDTAV | | | | | | | |
| | QPEDFATYYC | | | | | | | | YYCATYRSYVTP | | | | | | | |
| | QQYYAYPWTF | | | | | | | | LDYWGQGTLVT | | | | | | | |
| | GQGTKVEIK | | | | | | | | VSS | | | | | | | |
| CRLR | QSVLTQPPSVS | 1049 | SSNI | 1050 | DNN | 1051 | GTW | 1052 | QVQLVESGGGV | 1053 | GFTF | 1054 | ISFD | 1055 | ARDR | 1056 |
| | AAPGQKVTIS | | GNNY | | | | DSRL | | VQPGRSLRLSCA | | SSFG | | GSIK | | LNYY | |
| | CSGSSSNIGNN | | | | | | SAV | | ASGFTFSSFGMH | | | | | | DSSG | |
| | YVSWYQQLPG | | | | | | V | | WVRQAPGKGLE | | | | | | YYHY | |
| | TAPKLLIYDN | | | | | | | | WVAVISFDGSIK | | | | | | KYYG | |
| | NKRPSGIPDRF | | | | | | | | YSVDSVKGRFTIS | | | | | | MAV | |
| | SGSKSGTSTTL | | | | | | | | RDNSKNTLFLQM | | | | | | | |
| | GITGLQTGDE | | | | | | | | NSLRAEDTAVYY | | | | | | | |
| | ADYYCGTWD | | | | | | | | CARDRLNYYDSS | | | | | | | |
| | SRLSAVVFGG | | | | | | | | GYYHYKYYGMA | | | | | | | |
| | GTKLTVL | | | | | | | | VWGQGTTVTVSS | | | | | | | |
| Dabiga- | DVVMTQSPLS | 1057 | QSLL | 1058 | LVS | 1059 | LQST | 1060 | QVQLQESGPGLV | 1061 | GFSL | 1062 | IWAG | 1063 | ASAA | 1064 |
| tran | LPVTLGQPASI | | YTDG | | | | HFPH | | KPSETLSLTCTVS | | TSYI | | GST | | YYSY | |
| | SCKSSQSLLYT | | KTY | | | | T | | GFSLTSYIVDWIR | | | | | | YNYD | |
| | DGKTYLYWFL | | | | | | | | QPPGKGLEWIGV | | | | | | GFAY | |
| | QRPGQSPRRLI | | | | | | | | IWAGGSTGYNSA | | | | | | | |
| | YLVSKLDSGV | | | | | | | | LRSRVSITKDTSK | | | | | | | |
| | PDRFSGSGSGT | | | | | | | | NQFSLKLSSVTA | | | | | | | |
| | DFTLKISRVEA | | | | | | | | ADTAVYYCASA | | | | | | | |
| | EDVGVYYCLQ | | | | | | | | AYYSYYNYDGF | | | | | | | |
| | STHFPHTFGG | | | | | | | | AYWGQGTLVTV | | | | | | | |
| | GTKVEIK | | | | | | | | SS | | | | | | | |
| DLL3 | EIVMTQSPATL | 1065 | QSVS | 1066 | YAS | 1067 | QQD | 1068 | QVQLVQSGAEV | 1069 | GYTF | 1070 | INTY | 1071 | ARIG | 1072 |
| | SVSPGERATLS | | ND | | | | YTSP | | KKPGASVKVSCK | | TNY | | TGEP | | DSSP | |
| | CKASQSVSND | | | | | | WT | | ASGYTFTNYGM | | G | | | | SDY | |
| | VVWYQQKPG | | | | | | | | NWVRQAPGQGL | | | | | | | |
| | QAPRLLIYYAS | | | | | | | | EWMGWINTYTG | | | | | | | |
| | NRYTGIPARFS | | | | | | | | EPTYADDFKGRV | | | | | | | |
| | GSGSGTEFTLT | | | | | | | | TMTTDTSTSTAY | | | | | | | |
| | ISSLQSEDFAV | | | | | | | | MELRSLRSDDTA | | | | | | | |
| | YYCQQDYTSP | | | | | | | | VYYCARIGDSSPS | | | | | | | |
| | WTFGQGTKLE | | | | | | | | DYWGQGTLVTV | | | | | | | |
| | IK | | | | | | | | SS | | | | | | | |
| DLL4 | EIVLTQSPATL | 1073 | QSVS | 1074 | DAS | 1075 | QHRS | 1076 | QVQLVESGGGV | 1077 | GFTF | 1078 | LWYD | 1079 | ARDH | 1080 |
| | SLSPGERATLS | | SY | | | | NWP | | VQPGRSLRLSCA | | SSYG | | GTNK | | DFRS | |
| | CRASQSVSSY | | | | | | PT | | ASGFTFSSYGMH | | | | | | GYEG | |
| | LAWYQQKPG | | | | | | | | WVRQAPGKGLE | | | | | | WFDP | |
| | QAPRLLIYDAS | | | | | | | | WVSFLWYDGTN | | | | | | | |
| | NRATGIPARFS | | | | | | | | KNYVESVKGRFT | | | | | | | |
| | GSGSGTDFTLT | | | | | | | | ISRDNSKNMLYL | | | | | | | |
| | ISSLEPEDFAV | | | | | | | | EMNSLRAEDTAV | | | | | | | |
| | YYCQHRSNWP | | | | | | | | YYCARDHDFRSG | | | | | | | |
| | PTFGGGTKVEI | | | | | | | | YEGWFDPWGQG | | | | | | | |
| | K | | | | | | | | TLVTVSS | | | | | | | |
| DLL4 | DIVMTQSPDSL | 1081 | ESVD | 1082 | AAS | 1083 | QQS | 1084 | QVQLVQSGAEV | 1085 | GYSF | 1086 | ISSY | 1087 | ARDY | 1088 |
| | AVSLGERATIS | | NYGI | | | | KEVP | | KKPGASVKISCK | | TAY | | NGAT | | DYDV | |
| | CRASESVDNY | | SF | | | | WT | | ASGYSFTAYYIH | | Y | | | | GMDY | |
| | GISFMKWFQQ | | | | | | | | WVKQAPGQGLE | | | | | | | |
| | KPGQPPKLLIY | | | | | | | | WIGYISSYNGAT | | | | | | | |
| | AASNQGSGVP | | | | | | | | NYNQKFKGRVTF | | | | | | | |
| | DRFSGSGSGT | | | | | | | | TTDTSTSTAYME | | | | | | | |
| | DFTLTISSLQA | | | | | | | | LRSLRSDDTAVY | | | | | | | |
| | EDVAVYYCQ | | | | | | | | YCARDYDYDVG | | | | | | | |
| | QSKEVPWTFG | | | | | | | | MDYWGQGTLVT | | | | | | | |
| | GGTKVEIK | | | | | | | | VSS | | | | | | | |
| DNA/his | ENVLTQSPAI | 1089 | SSVS | 1090 | STS | 1091 | QQY | 1092 | QVQLKESGPGLV | 1093 | GFSL | 1094 | IWGG | 1095 | AKEK | 1096 |
| tone | MSASPGEKVT | | SSY | | | | SGYP | | APSQSLSITCTVS | | TDY | | GST | | RRGY | |
| (H1) | MTCRASSSVS | | | | | | LT | | GFSLTDYGVRWI | | G | | | | YYAM | |
| complex | SSYLHWYQQK | | | | | | | | RQPPGKGLEWLG | | | | | | DY | |
| | SGASPKLWIYS | | | | | | | | VIWGGGSTYYNS | | | | | | | |
| | TSNLASGVPA | | | | | | | | ALKSRLSISKDNS | | | | | | | |
| | RFSGSGSGTSY | | | | | | | | KSQVFLKMNSLQ | | | | | | | |
| | SLTISSVEAED | | | | | | | | TDDTAMYYCAK | | | | | | | |
| | AATYYCQQYS | | | | | | | | EKRRGYYYAMD | | | | | | | |
| | GYPLTFGGGT | | | | | | | | YWGQGTSVTVSS | | | | | | | |
| | KLEIK | | | | | | | | | | | | | | | |
| EGFR | DILLTQSPVILS | 1097 | QSIG | 1098 | YAS | 1099 | QQN | 1100 | QVQLKQSGPGLV | 1101 | GFSL | 1102 | IWSG | 1103 | ARAL | 1104 |
| | VSPGERVSFSC | | TN | | | | NNW | | QPSQSLSITCTVS | | TNY | | GNT | | TYYD | |
| | RASQSIGTNIH | | | | | | PTT | | GFSLTNYGVHW | | G | | | | YEFA | |
| | WYQQRTNGSP | | | | | | | | VRQSPGKGLEWL | | | | | | Y | |
| | RLLIKYASESIS | | | | | | | | GVIWSGGNTDYN | | | | | | | |
| | GIPSRFSGSGS | | | | | | | | TPFTSRLSINKDN | | | | | | | |
| | GTDFTLSINSV | | | | | | | | SKSQVFFKMNSL | | | | | | | |
| | ESEDIADYYC | | | | | | | | QSNDTAIYYCAR | | | | | | | |
| | QQNNNWPTTF | | | | | | | | ALTYYDYEFAY | | | | | | | |
| | GAGTKLELK | | | | | | | | WGQGTLVTVSA | | | | | | | |
| EGFR | DIQMTQSPSSL | 1105 | QDIS | 1106 | DAS | 1107 | QHF | 1108 | QVQLQESGPGLV | 1109 | GGS | 1110 | IYYS | 1111 | VRDR | 1112 |
| | SASVGDRVTIT | | NY | | | | DHLP | | KPSETLSLTCTVS | | VSSG | | GNT | | VTGA | |
| | CQASQDISNY | | | | | | LA | | GGSVSSGDYYW | | DYY | | | | FDI | |
| | LNWYQQKPG | | | | | | | | TWIRQSPGKGLE | | | | | | | |
| | KAPKLLIYDAS | | | | | | | | WIGHIYYSGNTN | | | | | | | |
| | NLETGVPSRFS | | | | | | | | YNPSLKSRLTISI | | | | | | | |
| | GSGSGTDFTFT | | | | | | | | DTSKTQFSLKLSS | | | | | | | |
| | ISSLQPEDIAT | | | | | | | | VTAADTAIYYCV | | | | | | | |
| | YFCQHFDHLP | | | | | | | | RDRVTGAFDIWG | | | | | | | |
| | LAFGGGTKVE | | | | | | | | QGTMVTVSS | | | | | | | |
| | IK | | | | | | | | | | | | | | | |
| EGFR | EIVMTQSPATL | 1113 | QSVS | 1114 | DAS | 1115 | HQY | 1116 | QVQLQESGPGLV | 1117 | GGSI | 1118 | IYYS | 1119 | ARVS | 1120 |
| | SLSPGERATLS | | SY | | | | GSTP | | KPSQTLSLTCTVS | | SSGD | | GST | | IFGV | |
| | CRASQSVSSY | | | | | | LT | | GGSISSGDYYWS | | YY | | | | GTFD | |
| | LAWYQQKPG | | | | | | | | WIRQPPGKGLEW | | | | | | Y | |
| | QAPRLLIYDAS | | | | | | | | IGYIYYSGSTDYN | | | | | | | |
| | NRATGIPARFS | | | | | | | | PSLKSRVTMSVD | | | | | | | |
| | GSGSGTDFTLT | | | | | | | | TSKNQFSLKVNS | | | | | | | |
| | ISSLEPEDFAV | | | | | | | | VTAADTAVYYC | | | | | | | |
| | YYCHQYGSTP | | | | | | | | ARVSIFGVGTFD | | | | | | | |
| | LTFGGGTKAEI | | | | | | | | YWGQGTLVTVSS | | | | | | | |
| | KR | | | | | | | | | | | | | | | |
| EGFR | DIQMTQSPSSL | 1121 | QNIV | 1122 | KVS | 1123 | FQYS | 1124 | QVQLQQSGAEV | 1125 | GYTF | 1126 | INPT | 1127 | ARQG | 1128 |
| | SASVGDRVTIT | | HSNG | | | | HVP | | KKPGSSVKVSCK | | TNY | | SGGS | | LWFD | |
| | CRSSQNIVHSN | | NTY | | | | WT | | ASGYTFTNYYIY | | Y | | | | SDGR | |
| | GNTYLDWYQ | | | | | | | | WVRQAPGQGLE | | | | | | GFDF | |
| | QTPGKAPKLLI | | | | | | | | WIGGINPTSGGSN | | | | | | W | |
| | YKVSNRFSGV | | | | | | | | FNEKFKTRVTITV | | | | | | | |
| | PSRFSGSGSGT | | | | | | | | DESTNTAYMELS | | | | | | | |
| | DFTFTISSLQPE | | | | | | | | SLRSEDTAFYFC | | | | | | | |
| | DIATYYCFQY | | | | | | | | ARQGLWFDSDG | | | | | | | |
| | SHVPWTFGQG | | | | | | | | RGFDFWGQGSTV | | | | | | | |
| | TKLQIT | | | | | | | | TVSS | | | | | | | |
| EGFR | IQLTQSPSSLS | 1129 | QDIS | 1130 | DAS | 1131 | QQF | 1132 | QVQLVESGGGV | 1133 | GFTF | 1134 | IWDD | 1135 | ARDG | 1136 |
| | ASVGDRVTIT | | SA | | | | NSYP | | VQPGRSLRLSCA | | STYG | | GSYK | | ITMV | |
| | CRASQDISSAL | | | | | | LT | | ASGFTFSTYGMH | | | | | | RGVM | |
| | VWYQQKPGK | | | | | | | | WVRQAPGKGLE | | | | | | KDYF | |
| | APKLLIYDASS | | | | | | | | WVAVIWDDGSY | | | | | | DY | |
| | LESGVPSARFS | | | | | | | | KYYGDSVKGRFT | | | | | | | |
| | GSESGTDFTLT | | | | | | | | ISRDNSKNTLYL | | | | | | | |
| | ISSLQPEDFAT | | | | | | | | QMNSLRAEDTA | | | | | | | |
| | YYCQQFNSYP | | | | | | | | VYYCARDGITMV | | | | | | | |
| | LTFGGGTKVEI | | | | | | | | RGVMKDYFDYW | | | | | | | |
| | K | | | | | | | | GQGTLVTVSS | | | | | | | |
| EGFR | DIQMTQSPSSL | 1137 | QDIN | 1138 | YTS | 1139 | LQY | 1140 | QVQLVQSGAEV | 1141 | GYTF | 1142 | IYPG | 1143 | ARYD | 1144 |
| | SASVGDRVTIT | | NY | | | | DNLL | | AKPGASVKLSCK | | TSY | | DGDT | | APGY | |
| | CRASQDINNY | | | | | | YT | | ASGYTFTSYWM | | W | | | | AMDY | |
| | LAWYQHKPG | | | | | | | | QWVKQRPGQGL | | | | | | | |
| | KGPKLLIHYTS | | | | | | | | ECIGTIYPGDGDT | | | | | | | |
| | TLHPGIPSRFS | | | | | | | | TYTQKFQGKATL | | | | | | | |
| | GSGSGRDYSF | | | | | | | | TADKSSSTAYMQ | | | | | | | |
| | SISSLEPEDIAT | | | | | | | | LSSLRSEDSAVY | | | | | | | |
| | YYCLQYDNLL | | | | | | | | YCARYDAPGYA | | | | | | | |
| | YTFGQGTKLEI | | | | | | | | MDYWGQGTLVT | | | | | | | |
| | K | | | | | | | | VSS | | | | | | | |
| EGFR | DIQMTQSPSSL | 1145 | SSVT | 1146 | DTS | 1147 | QQW | 1148 | QVQLVQSGAEV | 1149 | GYTF | 1150 | FNPS | 1151 | ASRD | 1152 |
| | SASVGDRVTIT | | Y | | | | SSHI | | KKPGASVKVSCK | | TSH | | NGRT | | YDYD | |
| | CSASSSVIYM | | | | | | FT | | ASGYTFTSHWM | | W | | | | GRYF | |
| | YWYQQKPGK | | | | | | | | HWVRQAPGQGL | | | | | | DY | |
| | APKLLIYDTSN | | | | | | | | EWIGEFNPSNGR | | | | | | | |
| | LASGVPSRFSG | | | | | | | | TNYNEKFKSKAT | | | | | | | |
| | SGSGTDYTFTI | | | | | | | | MTVDTSTNTAY | | | | | | | |
| | SSLQPEDIATY | | | | | | | | MELSSLRSEDTA | | | | | | | |
| | YCQQWSSHIF | | | | | | | | VYYCASRDYDY | | | | | | | |
| | TFGQGTKVEI | | | | | | | | DGRYFDYWGQG | | | | | | | |
| | K | | | | | | | | TLVTVSS | | | | | | | |
| EGFR | DIQMTQSPSSL | 1153 | QGIN | 1154 | NTN | 1155 | LQH | 1156 | QVQLVQSGAEV | 1157 | GFTF | 1158 | FNPN | 1159 | ARLS | 1160 |
| | SASVGDRVTIT | | NY | | | | NSFP | | KKPGSSVKVSCK | | TDY | | SGYS | | PGGY | |
| | CRASQGINNY | | | | | | T | | ASGFTFTDYKIH | | K | | | | YVMD | |
| | LNWYQQKPG | | | | | | | | WVRQAPGQGLE | | | | | | AW | |
| | KAPKRLIYNT | | | | | | | | WMGYFNPNSGY | | | | | | | |
| | NNLQTGVPSR | | | | | | | | STYAQKFQGRVT | | | | | | | |
| | FSGSGSGTEFT | | | | | | | | ITADKSTSTAYM | | | | | | | |
| | LTISSLQPEDF | | | | | | | | ELSSLRSEDTAV | | | | | | | |
| | ATYYCLQHNS | | | | | | | | YYCARLSPGGYY | | | | | | | |
| | FPTFGQGTKLE | | | | | | | | VMDAWGQGTTV | | | | | | | |
| | IK | | | | | | | | TVSS | | | | | | | |
| EGFR | DIQMTQSPSSL | 1161 | QNIA | 1162 | SAS | 1163 | QQSE | 1164 | EVQLVESGGGLV | 1165 | GFTL | 1166 | ISAA | 1167 | ARES | 1168 |
| HER3 | SASVGDRVTIT | | TD | | | | PEPY | | QPGGSLRLSCAA | | SGD | | GGYT | | RVSF | |
| | CRASQNIATD | | | | | | T | | SGFTLSGDWIHW | | W | | | | EAAM | |
| | VAWYQQKPG | | | | | | | | VRQAPGKGLEW | | | | | | DY | |
| | KAPKLLIYSAS | | | | | | | | VGEISAAGGYTD | | | | | | | |
| | FLYSGVPSRFS | | | | | | | | YADSVKGRFTIS | | | | | | | |
| | GSGSGTDFTLT | | | | | | | | ADTSKNTAYLQ | | | | | | | |
| | ISSLQPEDFAT | | | | | | | | MNSLRAEDTAV | | | | | | | |
| | YYCQQSEPEP | | | | | | | | YYCARESRVSFE | | | | | | | |
| | YTFGQGTKVE | | | | | | | | AAMDYWGQGTL | | | | | | | |
| | IK | | | | | | | | VTVSS | | | | | | | |
| EGFRvII | DILMTQSPSSM | 1169 | QDIN | 1170 | HGT | 1171 | VQY | 1172 | DVQLQESGPSLV | 1173 | GYSI | 1174 | ISYS | 1175 | VTAG | 1176 |
| I | SVSLGDTVSIT | | SN | | | | AQFP | | KPSQSLSLTCTVT | | TSDF | | GNT | | RGFP | |
| | CHSSQDINSNI | | | | | | WT | | GYSITSDFAWNW | | A | | | | Y | |
| | GWLQQRPGKS | | | | | | | | IRQFPGNKLEWM | | | | | | | |
| | FKGLIYHGTN | | | | | | | | GYISYSGNTRYN | | | | | | | |
| | LDDEVPSRFSG | | | | | | | | PSLKSRISITRDTS | | | | | | | |
| | SGSGADYSLTI | | | | | | | | KNQFFLQLNSVTI | | | | | | | |
| | SSLESEDFADY | | | | | | | | EDTATYYCVTAG | | | | | | | |
| | YCVQYAQFP | | | | | | | | RGFPYWGQGTL | | | | | | | |
| | WTFGGGTKLE | | | | | | | | VTVSS | | | | | | | |
| | IKA | | | | | | | | | | | | | | | |
| EGFRvII | DIQMTQSPSS | 1177 | QDIN | 1178 | HGT | 1179 | VQY | 1180 | QVQLQESGPGLV | 1181 | GYSI | 1182 | ISYS | 1183 | VTAG | 1184 |
| I | MSVSVGDRVT | | SN | | | | AQFP | | KPSQTLSLTCTVS | | SSDF | | GNT | | RGFP | |
| | ITCHSSQDINS | | | | | | WT | | GYSISSDFAWNW | | A | | | | Y | |
| | NIGWLQQKPG | | | | | | | | IRQPPGKGLEWM | | | | | | | |
| | KSFKGLIYHGT | | | | | | | | GYISYSGNTRYQ | | | | | | | |
| | NLDDGVPSRF | | | | | | | | PSLKSRITISRDTS | | | | | | | |
| | SGSGSGTDYT | | | | | | | | KNQFFLKLNSVT | | | | | | | |
| | LTISSLQPEDF | | | | | | | | AADTATYYCVT | | | | | | | |
| | ATYYCVQYA | | | | | | | | AGRGFPYWGQG | | | | | | | |
| | QFPWTFGGGT | | | | | | | | TLVTVSS | | | | | | | |
| | KLEIK | | | | | | | | | | | | | | | |
| EpCAM | ELVMTQSPSSL | 1185 | QSLL | 1186 | WAS | 1187 | QND | 1188 | EVQLLEQSGAEL | 1189 | ASG | 1190 | GDIH | 1191 | FCAR | 1192 |
| | TVTAGEKVTM | | NSGN | | | | YSYP | | VRPGTSVKISCK | | YAFT | | FPGS | | LRNW | |
| | SCKSSQSLLNS | | QKNY | | | | LT | | ASGYAFTNYWL | | N | | G | | DEPM | |
| | GNQKNYLTW | | | | | | | | GWVKQRPGHGL | | | | | | DY | |
| | YQQKPGQPPK | | | | | | | | EWIGDIHFPGSGN | | | | | | | |
| | LLIYWASTRES | | | | | | | | IHYNEKFKGKAT | | | | | | | |
| | GVPDRFTGSG | | | | | | | | LTADKSSSTAYM | | | | | | | |
| | SGTDFTLTISS | | | | | | | | QLSSLTFEDSAV | | | | | | | |
| | VQAEDLAVYY | | | | | | | | YFCARLRNWDEP | | | | | | | |
| | CQNDYSYPLT | | | | | | | | MDYWGQGTTVT | | | | | | | |
| | FGAGTKLEIK | | | | | | | | VSS | | | | | | | |
| EpCAM | ELQMTQSPSSL | 1193 | QSIS | 1194 | WAS | 1195 | QQS | 1196 | EVQLLESGGGVV | 1197 | GFTF | 1198 | ISYD | 1199 | AKDM | 1200 |
| | SASVGDRVTIT | | SY | | | | YDIP | | QPGRSLRLSCAA | | SSYG | | GSNK | | GWGS | |
| | CRTSQSISSYL | | | | | | YT | | SGFTFSSYGMHW | | | | | | GWRP | |
| | NWYQQKPGQ | | | | | | | | VRQAPGKGLEW | | | | | | YYYY | |
| | PPKLLIYWAST | | | | | | | | VAVISYDGSNKY | | | | | | GMDV | |
| | RESGVPDRFS | | | | | | | | YADSVKGRFTIS | | | | | | | |
| | GSGSGTDFTLT | | | | | | | | RDNSKNTLYLQ | | | | | | | |
| | ISSLQPEDSAT | | | | | | | | MNSLRAEDTAV | | | | | | | |
| | YYCQQSYDIP | | | | | | | | YYCAKDMGWGS | | | | | | | |
| | YTFGQGTKLEI | | | | | | | | GWRPYYYYGMD | | | | | | | |
| | K | | | | | | | | VWGQGTTVTVSS | | | | | | | |
| EpCAM | DIVLTQSPFSN | 1201 | KSLL | 1202 | QMS | 1203 | AQN | 1204 | QVKLQQSGPELK | 1205 | GYTF | 1206 | INTY | 1207 | ARFA | 1208 |
| | PVTLGTSASIS | | HSNG | | | | LEIP | | KPGETVKISCKAS | | TNY | | TGES | | IKGD | |
| | CRSTKSLLHSN | | ITY | | | | RT | | GYTFTNYGMNW | | G | | | | Y | |
| | GITYLYWYLQ | | | | | | | | VKQAPGKGLKW | | | | | | | |
| | KPGQSPQLLIY | | | | | | | | MGWINTYTGEST | | | | | | | |
| | QMSNLASGVP | | | | | | | | YADDFKGRFAFS | | | | | | | |
| | DRFSSSGSGTD | | | | | | | | LETSASAAYLQIN | | | | | | | |
| | FTLRISRVEAE | | | | | | | | NLKNEDTATYFC | | | | | | | |
| | DVGVYYCAQ | | | | | | | | ARFAIKGDYWG | | | | | | | |
| | NLEIPRTFGGG | | | | | | | | QGTTVTVSS | | | | | | | |
| | TKLEIK | | | | | | | | | | | | | | | |
| EpCAM | NIVMTQSPKS | 1209 | ENVV | 1210 | GAS | 1211 | GQG | 1212 | QVQLQQSGAELV | 1213 | GYA | 1214 | INPG | 1215 | ARDG | 1216 |
| | MSMSVGERVT | | TY | | | | YSYP | | RPGTSVKVSCKA | | FTNY | | SGGT | | PWFA | |
| | LTCKASENVV | | | | | | YT | | SGYAFTNYLIEW | | L | | | | Y | |
| | TYVSWYQQKP | | | | | | | | VKQRPGQGLEWI | | | | | | | |
| | EQSPKLLIYGA | | | | | | | | GVINPGSGGTNY | | | | | | | |
| | SNRYTGVPDR | | | | | | | | NEKFKGKATLTA | | | | | | | |
| | FTGSGSATDFT | | | | | | | | DKSSSTAYMQLS | | | | | | | |
| | LTISSVQAEDL | | | | | | | | SLTSDDSAVYFC | | | | | | | |
| | ADYHCGQGYS | | | | | | | | ARDGPWFAYWG | | | | | | | |
| | YPYTFGGGTK | | | | | | | | QGTLVTVSA | | | | | | | |
| | LEIK | | | | | | | | | | | | | | | |
| EpCAM | EIVMTQSPATL | 1217 | QSVS | 1218 | GAS | 1219 | QQY | 1220 | QVQLVQSGAEV | 1221 | SGGT | 1222 | GIIP | 1223 | CARG | 1224 |
| | SVSPGERATLS | | SN | | | | NNW | | KKPGSSVKVSCIC | | FSSY | | IFGT | | LLWN | |
| | CRASQSVSSN | | | | | | PPAY | | ASGGTFSSYAIS | | | | | | Y | |
| | LAWYQQKPG | | | | | | T | | WVRQAPGQGLE | | | | | | | |
| | QAPRLITYGAS | | | | | | | | WMGGIIPIFGTAN | | | | | | | |
| | TTASGIPARFS | | | | | | | | YAQKFQGRVTIT | | | | | | | |
| | ASGSGTDFTLT | | | | | | | | ADESTSTAYMEL | | | | | | | |
| | ISSLQSEDFAV | | | | | | | | SSLRSEDTAVYY | | | | | | | |
| | YYCQQYNNW | | | | | | | | CARGLLWNYWG | | | | | | | |
| | PPAYTFGQGT | | | | | | | | QGTLVTVSS | | | | | | | |
| | KLEIK | | | | | | | | | | | | | | | |
| EphA3 | DIQMTQSPSFL | 1225 | QGII | 1226 | AAS | 1227 | GQY | 1228 | QVQLVQSGAEV | 1229 | GYTF | 1230 | IYPG | 1231 | ARGG | 1232 |
| | SASVGDRVTIT | | SY | | | | ANY | | KKPGASVKVSCK | | TGY | | SGNT | | YYED | |
| | CRASQGIISYL | | | | | | PYT | | ASGYTFTGYWM | | W | | | | FDS | |
| | AWYQQKPEK | | | | | | | | NWVRQAPGQGL | | | | | | | |
| | APKRLIYAASS | | | | | | | | EWMGDIYPGSGN | | | | | | | |
| | LQSGVPSRFSG | | | | | | | | TNYDEKFQGRVT | | | | | | | |
| | SGSGTEFTLTI | | | | | | | | MTRDTSISTAYM | | | | | | | |
| | SSLQPEDFATY | | | | | | | | ELSRLRSDDTAV | | | | | | | |
| | YCGQYANYPY | | | | | | | | YYCARGGYYED | | | | | | | |
| | TFGQGTKLEIK | | | | | | | | FDSWGQGTTVTV | | | | | | | |
| | | | | | | | | | SS | | | | | | | |
| ERGT(G | DIQLTQTPLSL | 1233 | QSLV | 1234 | KVS | 1235 | SQST | 1236 | QVQLQQSGGGL | 1237 | GFTF | 1238 | IRNK | 1239 | SGGK | 1240 |
| alNAc) | PVSLGDQASIS | | HSNG | | | | HVPT | | VQPGGSMKIFCA | | SDA | | ANNH | | VRNA | |
| Tn | CRSSQSLVHS | | NTY | | | | | | ASGFTFSDAWM | | W | | ET | | Y | |
| Antigen | NGNTYLHWY | | | | | | | | DWVRQSPEKGLE | | | | | | | |
| | LQKPGQSPKL | | | | | | | | WVAEIRNKANN | | | | | | | |
| | LIYKVSNRFSG | | | | | | | | HETYYAESVKGR | | | | | | | |
| | VPDRFSGSGS | | | | | | | | FTITRDDSKSRMS | | | | | | | |
| | GTDFTLKISSV | | | | | | | | LQMNSLRAEDTG | | | | | | | |
| | EAEDLGVYFC | | | | | | | | IYYCSGGKVRNA | | | | | | | |
| | SQSTHVPTFG | | | | | | | | YWGQGTTVTVSS | | | | | | | |
| | GGTKLEIK | | | | | | | | | | | | | | | |
| FLT1 | EIVLTQSPGTL | 1241 | QSVS | 1242 | GAS | 1243 | QQY | 1244 | QAQVVESGGGV | 1245 | GFAF | 1246 | IWYD | 1247 | ARDH | 1248 |
| | SLSPGERATLS | | SSY | | | | GSSP | | VQSGRSLRLSCA | | SSYG | | GSNK | | YGSG | |
| | CRASQSVSSSY | | | | | | LT | | ASGFAFSSYGMH | | | | | | VHHY | |
| | LAWYQQKPG | | | | | | | | WVRQAPGKGLE | | | | | | FYYG | |
| | QAPRLLIYGAS | | | | | | | | WVAVIWYDGSN | | | | | | LDV | |
| | SRATGIPDRFS | | | | | | | | KYYADSVRGRFT | | | | | | | |
| | GSGSGTDFTLT | | | | | | | | ISRDNSENTLYLQ | | | | | | | |
| | ISRLEPEDFAV | | | | | | | | MNSLRAEDTAV | | | | | | | |
| | YYCQQYGSSP | | | | | | | | YYCARDHYGSG | | | | | | | |
| | LTFGGGTKVEI | | | | | | | | VHHYFYYGLDV | | | | | | | |
| | K | | | | | | | | WGQGTTVTVSS | | | | | | | |
| FOLR1 | DIQLTQSPSSL | 1249 | SSIS | 1250 | GTS | 1251 | QQW | 1252 | EVQLVESGGGVV | 1253 | GFTF | 1254 | ISSG | 1255 | ARHG | 1256 |
| | SASVGDRVTIT | | SNN | | | | SSYP | | QPGRSLRLSCSAS | | SGY | | GSYT | | DDPA | |
| | CSVSSSISSNN | | | | | | YMY | | GFTFSGYGLSWV | | G | | | | WFAY | |
| | LHWYQQKPG | | | | | | T | | RQAPGKGLEWV | | | | | | W | |
| | KAPKPWIYGT | | | | | | | | AMISSGGSYTYY | | | | | | | |
| | SNLASGVPSRF | | | | | | | | ADSVKGRFAISR | | | | | | | |
| | SGSGSGTDYT | | | | | | | | DNAKNTLFLQM | | | | | | | |
| | FTISSLQPEDIA | | | | | | | | DSLRPEDTGVYF | | | | | | | |
| | TYYCQQWSSY | | | | | | | | CARHGDDPAWF | | | | | | | |
| | PYMYTFGQGT | | | | | | | | AYWGQGTPVTV | | | | | | | |
| | KVEIK | | | | | | | | SS | | | | | | | |
| FOLR1 | DIVLTQSPLSL | 1257 | QSVS | 1258 | RAS | 1259 | QQSR | 1260 | QVQLVQSGAEV | 1261 | GYTF | 1262 | IHPY | 1263 | TRYD | 1264 |
| | AVSLGQPAIIS | | FAGT | | | | EYPY | | VKPGASVKISCK | | TGYF | | DGDT | | GSRA | |
| | CKASQSVSFA | | SL | | | | T | | ASGYTFTGYFMN | | | | | | MDY | |
| | GTSLMHWYH | | | | | | | | WVKQSPGQSLE | | | | | | | |
| | QKPGQQPRLLI | | | | | | | | WIGRIHPYDGDT | | | | | | | |
| | YRASNLEAGV | | | | | | | | FYNQKFQGKATL | | | | | | | |
| | PDRFSGSGSKT | | | | | | | | TVDKSSNTAHME | | | | | | | |
| | DFTLTISPVEA | | | | | | | | LLSLTSEDFAVY | | | | | | | |
| | EDAATYYCQQ | | | | | | | | YCTRYDGSRAM | | | | | | | |
| | SREYPYTFGG | | | | | | | | DYWGQGTTVTV | | | | | | | |
| | GTKLEIK | | | | | | | | SS | | | | | | | |
| frizzled | DIELTQPPSVS | 1265 | NIGS | 1266 | DKS | 1267 | QSY | 1268 | EVQLVESGGGLV | 1269 | GFTF | 1270 | ISGD | 1271 | ARNF | 1272 |
| family | VAPGQTARISC | | FY | | | | ANTL | | QPGGSLRLSCAA | | SHYT | | GSYT | | IKYV | |
| receptor | SGDNIGSFYV | | | | | | SLV | | SGFTFSHYTLSW | | | | | | FAN | |
| (FZD) | HWYQQKPGQ | | | | | | | | VRQAPGKGLEW | | | | | | | |
| | APVLVIYDKS | | | | | | | | VSVISGDGSYTY | | | | | | | |
| | NRPSGIPERFS | | | | | | | | YADSVKGRFTISS | | | | | | | |
| | GSNSGNTATL | | | | | | | | DNSKNTLYLQM | | | | | | | |
| | TISGTQAEDEA | | | | | | | | NSLRAEDTAVYY | | | | | | | |
| | DYYCQSYANT | | | | | | | | CARNFIKYVFAN | | | | | | | |
| | LSLVFGGGTK | | | | | | | | WGQGTLVTVSS | | | | | | | |
| | LTVLG | | | | | | | | | | | | | | | |
| Lewis Y | DVLMTQIPVS | 1273 | QIIV | 1274 | KVS | 1275 | FQGS | 1276 | EVNLVESGGGLV | 1277 | GFTF | 1278 | ISQG | 1279 | ARGL | 1280 |
| | LPVSLGDQASI | | HNNG | | | | HVPF | | QPGGSLKVSCVT | | SDY | | GDIT | | DDGA | |
| | SCRSSQIIVHN | | NTY | | | | T | | SGFTFSDYYMY | | Y | | | | WFAY | |
| | NGNTYLEWYL | | | | | | | | WVRQTPEKRLE | | | | | | | |
| | QKPGQSPQLLI | | | | | | | | WVAYISQGGDIT | | | | | | | |
| | YKVSNRFSGV | | | | | | | | DYPDTVKGRFTIS | | | | | | | |
| | PDRFSGSGSGT | | | | | | | | RDNAKNSLYLQ | | | | | | | |
| | DFTLKISRVEA | | | | | | | | MSRLKSEDTAM | | | | | | | |
| | EDLGVYYCFQ | | | | | | | | YYCARGLDDGA | | | | | | | |
| | GSHVPFTFGSG | | | | | | | | WFAYWGQGTLV | | | | | | | |
| | TKLEIK | | | | | | | | TVSV | | | | | | | |
| Lewis Y | DIQMTQSPSSL | 1281 | QRIV | 1282 | KVS | 1283 | FQGS | 1284 | EVQLVESGGGVV | 1285 | GFTF | 1286 | MSNV | 1287 | ARGT | 1288 |
| | SASVGDRVTIT | | HSNG | | | | HVPF | | QPGRSLRLSCSTS | | SDY | | GAIT | | RDGS | |
| | CRSSQRIVHSN | | NTY | | | | T | | GFTFSDYYMYW | | Y | | | | WFAY | |
| | GNTYLEWYQ | | | | | | | | VRQAPGKGLEW | | | | | | | |
| | QTPGKAPKLLI | | | | | | | | VAYMSNVGAITD | | | | | | | |
| | YKVSNRFSGV | | | | | | | | YPDTVKGRFTISR | | | | | | | |
| | PSRFSGSGSGT | | | | | | | | DNSKNTLFLQMD | | | | | | | |
| | DFTFTISSLQPE | | | | | | | | SLRPEDTGVYFC | | | | | | | |
| | DIATYYCFQG | | | | | | | | ARGTRDGSWFA | | | | | | | |
| | SHVPFTFGQG | | | | | | | | YWGQGTPVTVSS | | | | | | | |
| | TKLQIT | | | | | | | | | | | | | | | |
| Lewis X | DIVMTQAAFS | 1289 | KSLL | 1290 | QMS | 1291 | AQN | 1292 | EVKLLESGGGLV | 1293 | SGFD | 1294 | EINP | 1295 | CARE | 1296 |
| | NPVTLGTSASI | | YSNG | | | | LEVP | | QPGGSQKLSCAA | | FSGY | | DSST | | TGTR | |
| | SCRSSKSLLYS | | ITY | | | | WT | | SGFDFSGYWMS | | | | | | FDY | |
| | NGITYLYWYL | | | | | | | | WVRQAPGKGLE | | | | | | | |
| | QKPGQSPQLLI | | | | | | | | WIGEINPDSSTIN | | | | | | | |
| | YQMSNLASGV | | | | | | | | YTPSLKDKFIISR | | | | | | | |
| | PDRFSSSGSGT | | | | | | | | DNAKNTLYLQM | | | | | | | |
| | DFTLRISRVEA | | | | | | | | SKVRSEDTALYY | | | | | | | |
| | EDVGVYYCA | | | | | | | | CARETGTRFDYW | | | | | | | |
| | QNLEVPWTFG | | | | | | | | GQGTTLTVSS | | | | | | | |
| | GGTKLEIK | | | | | | | | | | | | | | | |
| GCGR | DIQMTQSPSSL | 1297 | QGIR | 1298 | AAS | 1299 | LQY | 1300 | EVQLVESGGGLV | 1301 | GFTF | 1302 | IQED | 1303 | AREP | 1304 |
| | SASVGDRVTIT | | ND | | | | NSNP | | QPGGSLRLSCAA | | SNYL | | GIEK | | SHYD | |
| | CRASQGIRND | | | | | | FT | | SGFTFSNYLMNW | | | | | | ILTG | |
| | LGWYQQKPG | | | | | | | | VRQAPGKGLEW | | | | | | YDYY | |
| | KAPKRLIYAA | | | | | | | | LANIQEDGIEKY | | | | | | YGMD | |
| | SSLQSGVPSRF | | | | | | | | YVDSVKGRFTIS | | | | | | V | |
| | SGSGSGTEFIL | | | | | | | | RDNAKNSLYLQ | | | | | | | |
| | TVSSLQPEDFA | | | | | | | | MNSLRAEDTAV | | | | | | | |
| | TYYCLQYNSN | | | | | | | | YYCAREPSHYDI | | | | | | | |
| | PFTFGPGTKV | | | | | | | | LTGYDYYYGMD | | | | | | | |
| | DIK | | | | | | | | VWGQGTTVTVSS | | | | | | | |
| GD2 | EIVMTQSPATL | 1305 | QSLV | 1306 | KVS | 1307 | SQST | 1308 | EVQLLQSGPELE | 1309 | GSSF | 1310 | IDPY | 1311 | VSGM | 1312 |
| | SVSPGERATLS | | HRNG | | | | HVPP | | KPGASVMISCKA | | TGY | | YGGT | | EY | |
| | CRSSQSLVHR | | NTY | | | | LT | | SGSSFTGYNMN | | N | | | | | |
| | NGNTYLHWY | | | | | | | | WVRQNIGKSLE | | | | | | | |
| | LQKPGQSPKL | | | | | | | | WIGAIDPYYGGT | | | | | | | |
| | LIHKVSNRFSG | | | | | | | | SYNQKFKGRATL | | | | | | | |
| | VPDRFSGSGS | | | | | | | | TVDKSSSTAYMH | | | | | | | |
| | GTDFTLKISRV | | | | | | | | LKSLTSEDSAVY | | | | | | | |
| | EAEDLGVYFC | | | | | | | | YCVSGMEYWGQ | | | | | | | |
| | SQSTHVPPLTF | | | | | | | | GTSVTVSS | | | | | | | |
| | GAGTKLELK | | | | | | | | | | | | | | | |
| GD2 | SIVMTQTPKFL | 1313 | QSVS | 1314 | SAS | 1315 | QQD | 1316 | QVQLKESGPGLV | 1317 | GFSV | 1318 | IWAG | 1319 | ASRG | 1320 |
| | LVSAGDRVTIT | | ND | | | | YSS | | APSQSLSITCTVS | | TNY | | GIT | | GHYG | |
| | CKASQSVSND | | | | | | | | GFSVTNYGVHW | | G | | | | YALD | |
| | VTWYQQKAG | | | | | | | | VRQPPGKGLEWL | | | | | | Y | |
| | QSPKLLIYSAS | | | | | | | | GVIWAGGITNYN | | | | | | | |
| | NRYSGVPDRF | | | | | | | | SAFMSRLSISKDN | | | | | | | |
| | TGSGYGTAFT | | | | | | | | SKSQVFLKMNSL | | | | | | | |
| | FTISTVQAEDL | | | | | | | | QIDDTAMYYCAS | | | | | | | |
| | AVYFCQQDYS | | | | | | | | RGGHYGYALDY | | | | | | | |
| | SFGGGTKLEIK | | | | | | | | WGQGTSVTVSS | | | | | | | |
| GD2 | EIVMTQTPATL | 1321 | QSVS | 1322 | SAS | 1323 | QQD | 1324 | QVQLVESGPGVV | 1325 | GFSV | 1326 | IWAG | 1327 | ASRG | 1328 |
| | SVSAGERVTIT | | ND | | | | YSS | | QPGRSLRISCAVS | | TNY | | GIT | | GHYG | |
| | CKASQSVSND | | | | | | | | GFSVTNYGVHW | | G | | | | YALD | |
| | VTWYQQKPG | | | | | | | | VRQPPGKGLEWL | | | | | | Y | |
| | QAPRLLIYSAS | | | | | | | | GVIWAGGITNYN | | | | | | | |
| | NRYSGVPARF | | | | | | | | SAFMSRLTISKDN | | | | | | | |
| | SGSGYGTEFTF | | | | | | | | SKNTVYLQMNSL | | | | | | | |
| | TISSVQSEDFA | | | | | | | | RAEDTAMYYCA | | | | | | | |
| | VYFCQQDYSS | | | | | | | | SRGGHYGYALD | | | | | | | |
| | FGQGTKLEIK | | | | | | | | YWGQGTLVTVSS | | | | | | | |
| GD2 o- | DVVMTQTPLS | 1329 | QSLL | 1330 | KVS | 1331 | SQST | 1332 | EVKLVESGGGLV | 1333 | KFTF | 1334 | IRNR | 1335 | ARVS | 1336 |
| acetyl | LPVSLGDQASI | | KNNG | | | | HIPY | | LPGDSLRLSCATS | | TDY | | ANGY | | NWAF | |
| | SCRSSQSLLKN | | NTF | | | | T | | KFTFTDYYMTW | | Y | | TT | | DY | |
| | NGNTFLHWYL | | | | | | | | VRQPPRKALEQL | | | | | | | |
| | QKSGQSPKLLI | | | | | | | | GFIRNRANGYTT | | | | | | | |
| | YKVSNRLSGV | | | | | | | | EYNPSVKGRFTIS | | | | | | | |
| | PDRFSGSGSGT | | | | | | | | RDNSQSILYLQM | | | | | | | |
| | YFTLKISRVEA | | | | | | | | NTLRTEDSATYY | | | | | | | |
| | EDLGVYFCSQ | | | | | | | | CARVSNWAFDY | | | | | | | |
| | STHIPYTFGGG | | | | | | | | WGQGTTLTVSS | | | | | | | |
| | TKLELK | | | | | | | | | | | | | | | |
| GD3 | DIQMTQITSSL | 1337 | GFTF | 1338 | ISSG | 1339 | TRG | 1340 | DVQLVESGGGLV | 1341 | QDIG | 1342 | YTS | 1343 | QQGK | 1344 |
| | SVSLGDRVIIS | | SNFG | | GSSI | | GTGT | | QPGGSRKLSCAA | | NF | | | | TLP | |
| | CRASQDIGNFL | | | | | | RSLY | | SGFTFSNFGMHW | | | | | | | |
| | NWYQQKPDG | | | | | | YFD | | VRQAPEKGLEW | | | | | | | |
| | SLKLLIYYTSR | | | | | | Y | | VAYISSGGSSINY | | | | | | | |
| | LQSGVPSRFSG | | | | | | | | ADTVKGRFTISR | | | | | | | |
| | WGSGTDYSLT | | | | | | | | DNPKNTLFLQMT | | | | | | | |
| | ISNLEEEDIATF | | | | | | | | SLRSEDTAIYYCT | | | | | | | |
| | FCQQGKTLPY | | | | | | | | RGGTGTRSLYYF | | | | | | | |
| | TFGGGTKLEIK | | | | | | | | DYWGQGATLIVS | | | | | | | |
| | | | | | | | | | S | | | | | | | |
| GD3 | DIQMTQTASS | 1345 | QDIS | 1346 | YSS | 1347 | HQY | 1348 | EVTLVESGGDFV | 1349 | GFAF | 1350 | ISSG | 1351 | TRVK | 1352 |
| | LPASLGDRVTI | | NY | | | | SKLP | | KPGGSLKVSCAA | | SHY | | GSGT | | LGTY | |
| | SCSASQDISNY | | | | | | WT | | SGFAFSHYAMSW | | A | | | | YFDS | |
| | LNWYQQKPD | | | | | | | | VRQTPAKRLEW | | | | | | | |
| | GTVKLLIFYSS | | | | | | | | VAYISSGGSGTY | | | | | | | |
| | NLHSGVPSRFS | | | | | | | | YSDSVKGRFTISR | | | | | | | |
| | GGGSGTDYSL | | | | | | | | DNAKNTLYLQM | | | | | | | |
| | TISNLEPEDIAT | | | | | | | | RSLRSEDSAMYF | | | | | | | |
| | YFCHQYSKLP | | | | | | | | CTRVKLGTYYFD | | | | | | | |
| | WTFGGGTKLE | | | | | | | | SWGQGTTLTVSS | | | | | | | |
| | IK | | | | | | | | | | | | | | | |
| GM1 | DIQMTQSPSSL | 1353 | QGIS | 1354 | AAS | 1355 | QQY | 1356 | EVQLVESGGGLV | 1357 | GFTF | 1358 | ISRS | 1359 | AGTV | 1360 |
| | SASVGDRVTIT | | SW | | | | NSYP | | QPGESLRLSCVA | | SRY | | GRDI | | TTYY | |
| | CRASQGISSW | | | | | | PT | | SGFTFSRYKMNW | | K | | | | YYFG | |
| | LAWYQQKPE | | | | | | | | VRQAPGKGLEW | | | | | | MDV | |
| | KAPKSLIYAAS | | | | | | | | VSYISRSGRDIYY | | | | | | | |
| | SLQSGVPSRFS | | | | | | | | ADSVKGRFTISR | | | | | | | |
| | GSGSGTDFTLT | | | | | | | | DNAKNSLYLQM | | | | | | | |
| | ISSLQPEDFAT | | | | | | | | NSLRDEDTAVYY | | | | | | | |
| | YYCQQYNSYP | | | | | | | | CAGTVTTYYYYF | | | | | | | |
| | PTFGGGTKVEI | | | | | | | | GMDVWGHGTTV | | | | | | | |
| | K | | | | | | | | TVSS | | | | | | | |
| GM1 | DIQMTQSPSSL | 1361 | QGIS | 1362 | AAS | 1363 | QQY | 1364 | EVQLVESGGGLY | 1365 | GFTF | 1366 | YISR | 1367 | CAGT | 1368 |
| fucosyl | SASVGDRVTIT | | W | | | | NSYP | | QPGESLRLSCVA | | SRYK | | SGRD | | VTTY | |
| | CRASQGISWL | | | | | | PT | | SGFTFSRYKMNW | | | | | | YYYF | |
| | AWYQQKPEK | | | | | | | | VRQAPGKGLLE | | | | | | GMDV | |
| | APKSLIYAASS | | | | | | | | WVSYISRSGRDIY | | | | | | | |
| | LQSGVPSRFSG | | | | | | | | YADSVKGRFTIS | | | | | | | |
| | SGSGTDFTLTI | | | | | | | | RDNAKNSLYLQ | | | | | | | |
| | SSLQPEDFATY | | | | | | | | MNSLRDEDTAV | | | | | | | |
| | YCQQYNSYPP | | | | | | | | YYCAGTVTTYY | | | | | | | |
| | TFGGGTKVEI | | | | | | | | YYFGMDVWGHG | | | | | | | |
| | K | | | | | | | | TTVTVSS | | | | | | | |
| GM1 | DIQMTQSPSSL | 1369 | QGIS | 1370 | AAS | 1371 | QQY | 1372 | EVQLVESGGGLV | 1373 | GFTF | 1374 | ISRS | 1375 | AGTV | 1376 |
| fucosyl | ASVGDRVTIT | | SW | | | | NSYP | | QPGESLRLSCVV | | SRY | | GRDI | | TTYY | |
| | CRASQGISSW | | | | | | PT | | SGFTFSRYKMNW | | K | | | | YYFG | |
| | LAWYQQKPE | | | | | | | | VRQAPGKGLEWI | | | | | | MDVW | |
| | KAPKSLIYAAS | | | | | | | | SYISRSGRDIYYA | | | | | | G | |
| | SLQSGVPSRFS | | | | | | | | DSVKGRFTISRD | | | | | | | |
| | GSGSGTDFTLT | | | | | | | | NAKNSLYLQMSS | | | | | | | |
| | ISCLQPEDFAT | | | | | | | | LRDEDTAVYYCA | | | | | | | |
| | YYCQQYNSYP | | | | | | | | GTVTTYYYYFG | | | | | | | |
| | PTFGGGTKVEI | | | | | | | | MDVWGLGITVT | | | | | | | |
| | K | | | | | | | | VSS | | | | | | | |
| GM1 | DIQMTQSPSSL | 1377 | QGIS | 1378 | AA | 1379 | QQY | 1380 | EVQLVESGGGSV | 1381 | GFTF | 1382 | ISRS | 1383 | AGTV | 1384 |
| fucosyl | SASVGDRVTIT | | SW | | | | NSYP | | QPGESLRLSCVA | | SRY | | GRDI | | TTYY | |
| | CRASQGISSW | | | | | | PT | | SGFTFSRYKMNW | | K | | | | YDFG | |
| | LAWYQQKPE | | | | | | | | VRQAPGKGLEW | | | | | | MDV | |
| | KAPKSLIYAAS | | | | | | | | VSYISRSGRDIYY | | | | | | | |
| | LQSGVPSRFSG | | | | | | | | ADSVKGRFTISR | | | | | | | |
| | SGSGTDFTLTI | | | | | | | | DNAKNSLYLQM | | | | | | | |
| | SSLQPEDFATY | | | | | | | | NSLRDEDTAVYY | | | | | | | |
| | YCQQYNSYPP | | | | | | | | CAGTVTTYYYDF | | | | | | | |
| | TFGGGTKVEI | | | | | | | | GMDVWGQGTTV | | | | | | | |
| | K | | | | | | | | TVSS | | | | | | | |
| GM2 | QIVLTQSPAIM | 1385 | SSVS | 1386 | STS | 1387 | QQRS | 1388 | EVQLQQSGPELV | 1389 | GYTF | 1390 | IYPN | 1391 | ATYG | 1392 |
| | SASPGEKVTIT | | Y | | | | SYPY | | KPGASVKISCKA | | TDY | | NGGT | | HYYG | |
| | CSASSSVSYM | | | | | | T | | SGYTFTDYNMD | | N | | | | YMFA | |
| | HWFQQKPGTS | | | | | | | | WVKQSHGKSLE | | | | | | Y | |
| | PKLWIYSTSNL | | | | | | | | WIGYIYPNNGGT | | | | | | | |
| | ASGVPARFSG | | | | | | | | GYNQKFKSKATL | | | | | | | |
| | SGSGTSYSLTI | | | | | | | | TVDKSSSTAYME | | | | | | | |
| | SRMEAEDAAT | | | | | | | | LHSLTSEDSAVY | | | | | | | |
| | YYCQQRSSYP | | | | | | | | YCATYGHYYGY | | | | | | | |
| | YTFGGGTKLEI | | | | | | | | MFAYWGQGTLV | | | | | | | |
| | K | | | | | | | | TVSA | | | | | | | |
| GPA33 | DIVMTQSQKF | 1393 | QNVR | 1394 | LAS | 1395 | LQH | 1396 | EVKLVESGGGLV | 1397 | GFAF | 1398 | ISSG | 1399 | APTT | 1400 |
| | MSTSVGDRVS | | TV | | | | WSY | | KPGGSLKLSCAA | | STYD | | GSYT | | VVPF | |
| | ITCKASQNVR | | | | | | PLT | | SGFAFSTYDMSW | | | | | | AY | |
| | TVVAWYQQK | | | | | | | | VRQTPEKRLEWV | | | | | | | |
| | PGQSPKTLIYL | | | | | | | | ATISSGGSYTYYL | | | | | | | |
| | ASNRHTGVPD | | | | | | | | DSVKGRFTISRDS | | | | | | | |
| | RFTGSGSGTDF | | | | | | | | ARNTLYLQMSSL | | | | | | | |
| | TLTISNVQSED | | | | | | | | RSEDTALYYCAP | | | | | | | |
| | LADYFCLQHW | | | | | | | | TTVVPFAYWGQ | | | | | | | |
| | SYPLTFGSGTK | | | | | | | | GTLVTVSA | | | | | | | |
| | LEVK | | | | | | | | | | | | | | | |
| GPNMB | EIVMTQSPATL | 1401 | QSVD | 1402 | GAS | 1403 | QQY | 1404 | QVQLQESGPGLV | 1405 | GGSI | 1406 | IYYS | 1407 | ARGY | 1408 |
| | SVSPGERATLS | | NN | | | | NNW | | KPSQTLSLTCTVS | | SSFN | | GST | | NWNY | |
| | CRASQSVDNN | | | | | | PPWT | | GGSISSFNYYWS | | YY | | | | FDY | |
| | LVWYQQKPG | | | | | | | | WIRHHPGKGLE | | | | | | | |
| | QAPRLLIYGAS | | | | | | | | WIGYIYYSGSTYS | | | | | | | |
| | TRATGIPARFS | | | | | | | | NPSLKSRVTISVD | | | | | | | |
| | GSGSGTEFTLT | | | | | | | | TSKNQFSLTLSSV | | | | | | | |
| | ISSLQSEDFAV | | | | | | | | TAADTAVYYCA | | | | | | | |
| | YYCQQYNNW | | | | | | | | RGYNWNYFDYW | | | | | | | |
| | PPWTFGQGTK | | | | | | | | GQGTLVTVSS | | | | | | | |
| | VEIK | | | | | | | | | | | | | | | |
| GUCY2 | EIVMTQSPATL | 1409 | QSVS | 1410 | GAS | 1411 | QQY | 1412 | QVQLQQWGAGL | 1413 | GGSF | 1414 | INHR | 1415 | ARER | 1416 |
| C | SVSPGERATLS | | RN | | | | KTW | | LKPSETLSLTCAV | | SGY | | GNT | | GYTY | |
| | CRASQSVSRN | | | | | | PRT | | FGGSFSGYYWS | | Y | | | | GNFD | |
| | LAWYQQKPG | | | | | | | | WIRQPPGKGLEW | | | | | | H | |
| | QAPRLLIYGAS | | | | | | | | IGEINHRGNTND | | | | | | | |
| | TRATGIPARFS | | | | | | | | NPSLKSRVTISVD | | | | | | | |
| | GSGSGTEFTLT | | | | | | | | TSKNQFALKLSS | | | | | | | |
| | IGSLQSEDFAV | | | | | | | | VTAADTAVYYC | | | | | | | |
| | YYCQQYKTW | | | | | | | | ARERGYTYGNFD | | | | | | | |
| | PRTFGQGTNV | | | | | | | | HWGQGTLVTVSS | | | | | | | |
| | EIK | | | | | | | | | | | | | | | |
| HER2 | DIQMTQSPSSL | 1417 | QDVN | 1418 | SAS | 1419 | QQA | 1420 | EVQLVESGGGLV | 1421 | GFNI | 1422 | IYPT | 1423 | SRWG | 1424 |
| | SASVGDRVTIT | | TA | | | | YTTP | | QPGGSLRLSCAA | | KDT | | NGYT | | GDGF | |
| | CRASQDVNTA | | | | | | PT | | SGFNIKDTYIHW | | Y | | | | YAMD | |
| | VAWYQQKPG | | | | | | | | VRQAPGKGLEW | | | | | | Y | |
| | KAPKLLIYSAS | | | | | | | | VARIYPTNGYTR | | | | | | | |
| | FLYSGVPSRFS | | | | | | | | YADSVKGRFTIS | | | | | | | |
| | GSRSGTDFTLT | | | | | | | | ADTSKNTAYLQ | | | | | | | |
| | ISSLQPEDFAT | | | | | | | | MNSLRAEDTAV | | | | | | | |
| | YYCQQAYTTP | | | | | | | | YYCSRWGGDGF | | | | | | | |
| | PTFGQGTKVEI | | | | | | | | YAMDYWGQGTL | | | | | | | |
| | K | | | | | | | | VTVSS | | | | | | | |
| HER2 | DIQMTQSPSSL | 1425 | QDVN | 1426 | SAS | 1427 | QQH | 1428 | EVQLVESGGGLV | 1429 | GFNI | 1430 | IYPT | 1431 | SRWG | 1432 |
| | SASVGDRVTIT | | TA | | | | YTTP | | QPGGSLRLSCAA | KDT | | NGYT | | GDGF | | |
| | CRASQDVNTA | | | | | | PT | | SGFNIKDTYIHW | | Y | | | | YAMD | |
| | VAWYQQKPG | | | | | | | | VRQAPGKGLEW | | | | | | Y | |
| | KAPKLLIYSAS | | | | | | | | VARIYPTNGYTR | | | | | | | |
| | FLYSGVPSRFS | | | | | | | | YADSVKGRFTIS | | | | | | | |
| | GSRSGTDFTLT | | | | | | | | ADTSKNTAYLQ | | | | | | | |
| | ISSLQPEDFAT | | | | | | | | MNSLRAEDTAV | | | | | | | |
| | YYCQQHYTTP | | | | | | | | YYCSRWGGDGF | | | | | | | |
| | PTFGQGTKVEI | | | | | | | | YAMDYWGQGTL | | | | | | | |
| | K | | | | | | | | VTVSS | | | | | | | |
| HER2 | DIQMTQSPSSL | 1433 | QDVS | 1434 | SAS | 1435 | QQY | 1436 | EVQLVESGGGLV | 1437 | GFTF | 1438 | VNPN | 1439 | ARNL | 1440 |
| | SASVGDRVTIT | | IG | | | | YIYP | | QPGGSLRLSCAA | | TDY | | SGGS | | GPSF | |
| | CKASQDVSIG | | | | | | YT | | SGFTFTDYTMDW | | T | | | | YFDY | |
| | VAWYQQKPG | | | | | | | | VRQAPGKGLEW | | | | | | W | |
| | KAPKLLIYSAS | | | | | | | | VADVNPNSGGSI | | | | | | | |
| | YRYTGVPSRF | | | | | | | | YNQRFKGRFTLS | | | | | | | |
| | SGSGSGTDFTL | | | | | | | | VDRSKNTLYLQ | | | | | | | |
| | TISSLQPEDFA | | | | | | | | MNSLRAEDTAV | | | | | | | |
| | TYYCQQYYIY | | | | | | | | YYCARNLGPSFY | | | | | | | |
| | PYTFGQGTKV | | | | | | | | FDYWGQGTLVT | | | | | | | |
| | EIK | | | | | | | | VSS | | | | | | | |
| HER2 | QSVLTQPPSVS | 1441 | SSNI | 1442 | GNT | 1443 | QSY | 1444 | QVQLVESGGGLV | 1445 | GFTF | 1446 | ISGR | 1447 | AKMT | 1448 |
| | GAPGQRVTISC | | GAGY | | | | DSSL | | QPGGSLRLSCAA | | RSY | | GDNT | | SNAF | |
| | TGSSSNIGAGY | | G | | | | SGW | | SGFTFRSYAMSW | | A | | | | AFDY | |
| | GVHWYQQLP | | | | | | V | | VRQAPGKGLEW | | | | | | | |
| | GTAPKLLIYG | | | | | | | | VSAISGRGDNTY | | | | | | | |
| | NTNRPSGVPD | | | | | | | | YADSVKGRFTIS | | | | | | | |
| | RFSGFKSGTSA | | | | | | | | RDNSKNTLYLQ | | | | | | | |
| | SLAITGLQAED | | | | | | | | MNSLRAEDTAV | | | | | | | |
| | EADYYCQSYD | | | | | | | | YYCAKMTSNAF | | | | | | | |
| | SSLSGWVFGG | | | | | | | | AFDYWGQGTLV | | | | | | | |
| | GTKLTVL | | | | | | | | TVSS | | | | | | | |
| HER3 | QSALTQPASV | 1449 | SSDV | 1450 | EVS | 1451 | CSYA | 1452 | EVQLLESGGGLV | 1453 | GFTF | 1454 | ISSS | 1455 | TRGL | 1456 |
| | SGSPGQSITISC | | GSYN | | | | GSSI | | QPGGSLRLSCAA | | SHY | | GGWT | | KMAT | |
| | TGTSSDVGSY | | V | | | | FVI | | SGFTFSHYVMA | | V | | | | IFDY | |
| | NVVSWYQQH | | | | | | | | WVRQAPGKGLE | | | | | | | |
| | PGKAPKLIIYE | | | | | | | | WVSSISSSGGWT | | | | | | | |
| | VSQRPSGVSN | | | | | | | | LYADSVKGRFTIS | | | | | | | |
| | RFSGSKSGNT | | | | | | | | RDNSKNTLYLQ | | | | | | | |
| | ASLTISGLQTE | | | | | | | | MNSLRAEDTAV | | | | | | | |
| | DEADYYCCSY | | | | | | | | YYCTRGLKMATI | | | | | | | |
| | AGSSIFVIFGG | | | | | | | | FDYWGQGTLVT | | | | | | | |
| | GTKVTVL | | | | | | | | VSS | | | | | | | |
| HER3 | DIQMTQSPSSL | 1457 | QGIS | 1458 | GAS | 1459 | QQY | 1460 | EVQLLESGGGLV | 1461 | GFTF | 1462 | INSQ | 1463 | ARWG | 1464 |
| | SASVGDRVTIT | | NW | | | | SSFP | | QPGGSLRLSCAA | | SSYA | | GKST | | DEGF | |
| | CRASQGISNW | | | | | | TT | | SGFTFSSYAMSW | | | | | | DI | |
| | LAWYQQKPG | | | | | | | | VRQAPGKGLEW | | | | | | | |
| | KAPKLLIYGAS | | | | | | | | VSAINSQGKSTY | | | | | | | |
| | SLQSGVPSRFS | | | | | | | | YADSVKGRFTIS | | | | | | | |
| | GSGSGTDFTLT | | | | | | | | RDNSKNTLYLQ | | | | | | | |
| | ISSLQPEDFAT | | | | | | | | MNSLRAEDTAV | | | | | | | |
| | YYCQQYSSFP | | | | | | | | YYCARWGDEGF | | | | | | | |
| | TTFGQGTKVEI | | | | | | | | DIWGQGTLVTVS | | | | | | | |
| | K | | | | | | | | S | | | | | | | |
| HER3 | DIEMTQSPDSL | 1465 | QSVL | 1466 | WAS | 1467 | QQY | 1468 | QVQLQQWGAGL | 1469 | GGSF | 1470 | INHS | 1471 | ARDK | 1472 |
| | AVSLGERATIN | | YSSS | | | | YSTP | | LKPSETLSLTCAV | | SGY | | GST | | WTWY | |
| | CRSSQSVLYSS | | NRNY | | | | RT | | YGGSFSGYYWS | | Y | | | | FDL | |
| | SNRNYLAWY | | | | | | | | WIRQPPGKGLEW | | | | | | | |
| | QQNPGQPPKL | | | | | | | | IGEINHSGSTNYN | | | | | | | |
| | LIYWASTRES | | | | | | | | PSLKSRVTISVET | | | | | | | |
| | GVPDRFSGSG | | | | | | | | SKNQFSLKLSSVT | | | | | | | |
| | SGTDFTLTISS | | | | | | | | AADTAVYYCAR | | | | | | | |
| | LQAEDVAVYY | | | | | | | | DKWTWYFDLWG | | | | | | | |
| | CQQYYSTPRT | | | | | | | | RGTLVTVSS | | | | | | | |
| | FGQGTKVEIK | | | | | | | | | | | | | | | |
| HER3 | DIVMTQSPDSL | 1473 | QSVL | 1474 | WAS | 1475 | QSD | 1476 | QVQLVQSGAEV | 1477 | GYTF | 1478 | IYAG | 1479 | ARHR | 1480 |
| | AVSLGERATIN | | NSGN | | | | YSYP | | KKPGASVKVSCK | | RSSY | | TGSP | | DYYS | |
| | CKSSQSVLNS | | QKNY | | | | YT | | ASGYTFRSSYISW | | | | | | NSLT | |
| | GNQKNYLTW | | | | | | | | VRQAPGQGLEW | | | | | | Y | |
| | YQQKPGQPPK | | | | | | | | MGWIYAGTGSPS | | | | | | | |
| | LLIYWASTRES | | | | | | | | YNQKLQGRVTM | | | | | | | |
| | GVPDRFSGSG | | | | | | | | TTDTSTSTAYME | | | | | | | |
| | SGTDFTLTISS | | | | | | | | LRSLRSDDTAVY | | | | | | | |
| | LQAEDVAVYY | | | | | | | | YCARHRDYYSNS | | | | | | | |
| | CQSDYSYPYT | | | | | | | | LTYWGQGTLVT | | | | | | | |
| | FGQGTKLEIK | | | | | | | | VSS | | | | | | | |
| HER3 | YELTQDPAVS | 1481 | SLRS | 1482 | GKN | 1483 | NSRD | 1484 | QVQLVQSGGGL | 1485 | GFTF | 1486 | ISWD | 1487 | ARDL | 1488 |
| | VALGQTVRIT | | YY | | | | SPGN | | VQPGGSLRLSCA | | DDY | | SGST | | GAYQ | |
| | CQGDSLRSYY | | | | | | QWV | | ASGFTFDDYAMH | | A | | | | WVEG | |
| | ASWYQQKPG | | | | | | | | WVRQAPGKGLE | | | | | | FDY | |
| | QAPVLVIYGK | | | | | | | | WVAGISWDSGST | | | | | | | |
| | NNRPSGIPDRF | | | | | | | | GYADSVKGRFTI | | | | | | | |
| | SGSTSGNSASL | | | | | | | | SRDNAKNSLYLQ | | | | | | | |
| | TITGAQAEDE | | | | | | | | MNSLRAEDTALY | | | | | | | |
| | ADYYCNSRDS | | | | | | | | YCARDLGAYQW | | | | | | | |
| | PGNQWVFGG | | | | | | | | VEGFDYWGQGT | | | | | | | |
| | GTKVTVL | | | | | | | | LVTVSS | | | | | | | |
| HGFR | DIVMTQSPDSL | 1489 | ESVD | 1490 | RAS | 1491 | QQS | 1492 | QVQLVQSGAEV | 1493 | GYIF | 1494 | IKPN | 1495 | ARSE | 1496 |
| (cMET) | AVSLGERATIN | | SYAN | | | | KEDP | | KKPGASVKVSCK | | TAY | | NGLA | | ITTE | |
| | CKSSESVDSY | | SF | | | | LT | | ASGYIFTAYTMH | | T | | | | FDY | |
| | ANSFLHWYQQ | | | | | | | | WVRQAPGQGLE | | | | | | | |
| | KPGQPPKLLIY | | | | | | | | WMGWIKPNNGL | | | | | | | |
| | RASTRESGVP | | | | | | | | ANYAQKFQGRV | | | | | | | |
| | DRFSGSGSGT | | | | | | | | TMTRDTSISTAY | | | | | | | |
| | DFTLTISSLQA | | | | | | | | MELSRLRSDDTA | | | | | | | |
| | EDVAVYYCQ | | | | | | | | VYYCARSEITTEF | | | | | | | |
| | QSKEDPLTFG | | | | | | | | DYWGQGTLVTV | | | | | | | |
| | GGTKVEIK | | | | | | | | SS | | | | | | | |
| HGFR | DIQMTQSPSSL | 1497 | SSVS | 1498 | STS | 1499 | QVY | 1500 | QVQLVQSGAEV | 1501 | GYTF | 1502 | VNPN | 1503 | ARAN | 1504 |
| (cMET) | SASVGDRVTIT | | SIY | | | | SGYP | | KKPGASVKVSCK | | TDY | | RRGT | | WLDY | |
| | CSVSSSVSSIY | | | | | | LT | | ASGYTFTDYYM | | Y | | | | | |
| | LHWYQQKPG | | | | | | | | HWVRQAPGQGL | | | | | | | |
| | KAPKLLIYSTS | | | | | | | | EWMGRVNPNRR | | | | | | | |
| | NLASGVPSRFS | | | | | | | | GTTYNQKFEGRV | | | | | | | |
| | GSGSGTDFTLT | | | | | | | | TMTTDTSTSTAY | | | | | | | |
| | ISSLQPEDFAT | | | | | | | | MELRSLRSDDTA | | | | | | | |
| | YYCQVYSGYP | | | | | | | | VYYCARANWLD | | | | | | | |
| | LTFGGGTKVEI | | | | | | | | YWGQGTTVTVSS | | | | | | | |
| | K | | | | | | | | | | | | | | | |
| IgHe | DIQLTQSPSSL | 1505 | QSVD | 1506 | AAS | 1507 | QQS | 1508 | EVQLVESGGGLV | 1509 | GYSI | 1510 | ITYD | 1511 | ARGS | 1512 |
| | SASVGDRVTIT | | YDGD | | | | HEDP | | QPGGSLRLSCAV | | TSGY | | GST | | HYFG | |
| | CRASQSVDYD | | SY | | | | YT | | SGYSITSGYSWN | | S | | | | HWHF | |
| | GDSYMNWYQ | | | | | | | | WIRQAPGKGLE | | | | | | AV | |
| | QKPGKAPKLLI | | | | | | | | WVASITYDGSTN | | | | | | | |
| | YAASYLESGV | | | | | | | | YNPSVKGRITISR | | | | | | | |
| | PSRFSGSGSGT | | | | | | | | DDSKNTFYLQM | | | | | | | |
| | DFTLTISSLQP | | | | | | | | NSLRAEDTAVYY | | | | | | | |
| | EDFATYYCQQ | | | | | | | | CARGSHYFGHW | | | | | | | |
| | SHEDPYTFGQ | | | | | | | | HFAVWGQGTLV | | | | | | | |
| | GTKVEIK | | | | | | | | TVSS | | | | | | | |
| IgHe | EIVMTQSPATL | 1513 | QSIG | 1514 | YAS | 1515 | QQS | 1516 | QVQLVQSGAEV | 1517 | GYTF | 1518 | IDPG | 1519 | ARFS | 1520 |
| | SVSPGERATLS | | TN | | | | WSW | | MKPGSSVKVSCK | | SWY | | TFTT | | HFSG | |
| | CRASQSIGTNI | | | | | | PTT | | ASGYTFSWYWL | | W | | | | SNYD | |
| | HWYQQKPGQ | | | | | | | | EWVRQAPGHGL | | | | | | YFDY | |
| | APRLLIYYASE | | | | | | | | EWMGEIDPGTFT | | | | | | W | |
| | SISGIPARFSGS | | | | | | | | TNYNEKFKARVT | | | | | | | |
| | GSGTEFTLTIS | | | | | | | | FTADTSTSTAYM | | | | | | | |
| | SLQSEDFAVY | | | | | | | | ELSSLRSEDTAV | | | | | | | |
| | YCQQSWSWPT | | | | | | | | YYCARFSHFSGS | | | | | | | |
| | TFGGGTKVEI | | | | | | | | NYDYFDYWGQG | | | | | | | |
| | K | | | | | | | | TLVTVSS | | | | | | | |
| IGLF2 | QSVLTQPPSVS | 1521 | SSNI | 1522 | DNN | 1523 | ETW | 1524 | QVQLVQSGAEV | 1525 | GYTF | 1526 | MNPN | 1527 | ARDP | 1528 |
| | AAPGQKVTIS | | ENNH | | | | DTSL | | KKPGASVKVSCK | | TSYD | | SGNT | | YYYY | |
| | CSGSSSNIENN | | | | | | SAGR | | ASGYTFTSYDIN | | | | | | YGMD | |
| | HVSWYQQLPG | | | | | | V | | WVRQATGQGLE | | | | | | V | |
| | TAPKLLIYDN | | | | | | | | WMGWMNPNSG | | | | | | | |
| | NKRPSGIPDRF | | | | | | | | NTGYAQKFQGR | | | | | | | |
| | SGSKSGTSATL | | | | | | | | VTMTRNTSISTA | | | | | | | |
| | GITGLQTGDE | | | | | | | | YMELSSLRSEDT | | | | | | | |
| | ADYYCETWD | | | | | | | | AVYYCARDPYY | | | | | | | |
| | TSLSAGRVFG | | | | | | | | YYYGMDVWGQ | | | | | | | |
| | GGTKLTVL | | | | | | | | GTTVTVSS | | | | | | | |
| Kalli- | DIQMTQSPSTL | 1529 | QSIS | 1530 | KAS | 1531 | QQY | 1532 | EVQLLESGGGLV | 1533 | GFTF | 1534 | IYSS | 1535 | AYRR | 1536 |
| kreins | SASVGDRVTIT | | SW | | | | NTY | | QPGGSLRLSCAA | | SHYI | | GGIT | | IGVP | |
| | CRASQSISSWL | | | | | | WT | | SGFTFSHYIMMW | | | | | | RRDE | |
| | AWYQQKPGK | | | | | | | | VRQAPGKGLEW | | | | | | FDI | |
| | APKLLIYKAST | | | | | | | | VSGIYSSGGITVY | | | | | | | |
| | LESGVPSRFSG | | | | | | | | ADSVKGRFTISR | | | | | | | |
| | SGSGTEFTLTI | | | | | | | | DNSKNTLYLQM | | | | | | | |
| | SSLQPDDFAT | | | | | | | | NSLRAEDTAVYY | | | | | | | |
| | YYCQQYNTY | | | | | | | | CAYRRIGVPRRD | | | | | | | |
| | WTFGQGTKVE | | | | | | | | EFDIWGQGTMVT | | | | | | | |
| | IK | | | | | | | | VSS | | | | | | | |
| KIRDL1/ | EIVLTQSPVTL | 1537 | QSVS | 1538 | DAS | 1539 | QQRS | 1540 | QVQLVQSGAEV | 1541 | GGTF | 1542 | FIPI | 1543 | ARIP | 1544 |
| 2/3 | SLSPGERATLS | | SY | | | | NWM | | KKPGSSVKVSCK | | SFYA | | FGAA | | SGSY | |
| | CRASQSVSSY | | | | | | YT | | ASGGTFSFYAIS | | | | | | YYDY | |
| | LAWYQQKPG | | | | | | | | WVRQAPGQGLE | | | | | | DMDV | |
| | QAPRLLIYDAS | | | | | | | | WMGGFIPIFGAA | | | | | | | |
| | NRATGIPARFS | | | | | | | | NYAQKFQGRVTI | | | | | | | |
| | GSGSGTDFTLT | | | | | | | | TADESTSTAYME | | | | | | | |
| | ISSLEPEDFAV | | | | | | | | LSSLRSDDTAVY | | | | | | | |
| | YYCQQRSNW | | | | | | | | YCARIPSGSYYY | | | | | | | |
| | MYTFGQGTKL | | | | | | | | DYDMDVWGQGT | | | | | | | |
| | EIK | | | | | | | | TVTVSS | | | | | | | |
| LINGO1 | DIQMTQSPAT | 1545 | QSVS | 1546 | DAS | 1547 | QQRS | 1548 | EVQLLESGGGLV | 1549 | GFTF | 1550 | IGPS | 1551 | ATEG | 1552 |
| | LSLSPGERATL | | SY | | | | NWP | | QPGGSLRLSCAA | | SAYE | | GGFT | | DNDA | |
| | SCRASQSVSSY | | | | | | MYT | | SGFTFSAYEMKW | | | | | | FDI | |
| | LAWYQQKPG | | | | | | | | VRQAPGKGLEW | | | | | | | |
| | QAPRLLIYDAS | | | | | | | | VSVIGPSGGFTFY | | | | | | | |
| | NRATGIPARFS | | | | | | | | ADSVKGRFTISR | | | | | | | |
| | GSGSGTDFTLT | | | | | | | | DNSKNTLYLQM | | | | | | | |
| | ISSLEPEDFAV | | | | | | | | NSLRAEDTAVYY | | | | | | | |
| | YYCQQRSNWP | | | | | | | | CATEGDNDAFDI | | | | | | | |
| | MYTFGQGTKL | | | | | | | | WGQGTTVTVSS | | | | | | | |
| | EIK | | | | | | | | | | | | | | | |
| LOXL2 | DIVMTQTPLSL | 1553 | KSLL | 1554 | RMS | 1555 | MQH | 1556 | QVQLVQSGAEV | 1557 | GYA | 1558 | INPG | 1559 | ARNW | 1560 |
| | SVTPGQPASIS | | HSNG | | | | LEYP | | KKPGASVKVSCK | | FTYY | | SGGT | | MNFD | |
| | CRSSKSLLHSN | | NTY | | | | YT | | ASGYAFTYYLIE | | L | | | | Y | |
| | GNTYLYWFLQ | | | | | | | | WVRQAPGQGLE | | | | | | | |
| | KPGQSPQFLIY | | | | | | | | WIGVINPGSGGT | | | | | | | |
| | RMSNLASGVP | | | | | | | | NYNEKFKGRATI | | | | | | | |
| | DRFSGSGSGT | | | | | | | | TADKSTSTAYME | | | | | | | |
| | DFTLKISRVEA | | | | | | | | LSSLRSEDTAVYF | | | | | | | |
| | EDVGVYYCM | | | | | | | | CARNWMNFDY | | | | | | | |
| | QHLEYPYTFG | | | | | | | | WGQGTTVTVSS | | | | | | | |
| | GGTKVEIK | | | | | | | | | | | | | | | |
| Ly6/PLA | ESVLTQPPSVS | 1561 | SSNI | 1562 | DNN | 1563 | AAW | 1564 | EVQLLESGGGLV | 1565 | GFTF | 1566 | ISSS | 1567 | AREG | 1568 |
| UR | GAPGQRVTISC | | GAGY | | | | DDR | | QPGGSLRLSCAA | | SNA | | GSTI | | LWAF | |
| domain- | TGSSSNIGAGY | | V | | | | LNGP | | SGFTFSNAWMS | | W | | | | DY | |
| con- | VVHWYQQLP | | | | | | V | | WVRQAPGKGLE | | | | | | | |
| taining | GTAPKLLIYD | | | | | | | | WVSYISSSGSTIY | | | | | | | |
| protein | NNKRPSGVPD | | | | | | | | YADSVKGRFTIS | | | | | | | |
| 3 | RFSGSKSGTSA | | | | | | | | RDNSKNTLYLQ | | | | | | | |
| | SLAISGLRSED | | | | | | | | MNSLRAEDTAV | | | | | | | |
| | EADYYCAAW | | | | | | | | YYCAREGLWAF | | | | | | | |
| | DDRLNGPVFG | | | | | | | | DYWGQGTLVTV | | | | | | | |
| | GGTKLTVL | | | | | | | | SS | | | | | | | |
| MADCA | DIVMTQTPLSL | 1569 | QSLL | 1570 | EVS | 1571 | MQNI | 1572 | QVQLVQSGAEV | 1573 | GYTF | 1574 | ISVY | 1575 | AREG | 1576 |
| M1 | SVTPGQPASIS | | HTDG | | | | QLP | | KKPGASVKVSCK | | TSYG | | SGNT | | SSSS | |
| | CKSSQSLLHT | | TTY | | | | WT | | ASGYTFTSYGIN | | | | | | GDYY | |
| | DGTTYLYWYL | | | | | | | | WVRQAPGQGLE | | | | | | YGMD | |
| | QKPGQPPQLLI | | | | | | | | WMGWISVYSGN | | | | | | V | |
| | YEVSNRFSGV | | | | | | | | TNYAQKVQGRV | | | | | | | |
| | PDRFSGSGSGT | | | | | | | | TMTADTSTSTAY | | | | | | | |
| | DFTLKISRVEA | | | | | | | | MDLRSLRSDDTA | | | | | | | |
| | EDVGIYYCMQ | | | | | | | | VYYCAREGSSSS | | | | | | | |
| | NIQLPWTFGQ | | | | | | | | GDYYYGMDVW | | | | | | | |
| | GTKVEIK | | | | | | | | GQGTTVTVSS | | | | | | | |
| MAG | DIVMTQSPDSL | 1577 | HSVL | 1578 | WAS | 1579 | HQY | 1580 | QVQLVQSGSELK | 1581 | GYTF | 1582 | INTY | 1583 | ARNP | 1584 |
| | AVSLGERATIN | | YSSN | | | | LSSL | | KPGASVKVSCKA | | TNY | | TGEP | | INYY | |
| | CKSSHSVLYSS | | QKNY | | | | T | | SGYTFTNYGMN | | G | | | | GINY | |
| | NQKNYLAWY | | | | | | | | WVRQAPGQGLE | | | | | | EGYV | |
| | QQKPGQPPKL | | | | | | | | WMGWINTYTGE | | | | | | MDY | |
| | LIYWASTRES | | | | | | | | PTYADDFTGRFV | | | | | | | |
| | GVPDRFSGSG | | | | | | | | FSLDTSVSTAYL | | | | | | | |
| | SGTDFTLTISS | | | | | | | | QISSLKAEDTAV | | | | | | | |
| | LQAEDVAVYY | | | | | | | | YYCARNPINYYG | | | | | | | |
| | CHQYLSSLTF | | | | | | | | INYEGYVMDYW | | | | | | | |
| | GQGTKLEIK | | | | | | | | GQGTLVTVSS | | | | | | | |
| Meso- | DIALTQPASVS | 1585 | SSDI | 1586 | GVN | 1587 | SSYD | 1588 | QVELVQSGAEVK | 1589 | GYSF | 1590 | IDPG | 1591 | ARGQ | 1592 |
| thelin | GSPGQSITISCT | | GGYN | | | | IESA | | KPGESLKISCKGS | | TSY | | DSRT | | LYGG | |
| | GTSSDIGGYNS | | S | | | | TPV | | GYSFTSYWIGWV | | W | | | | TYMD | |
| | VSWYQQHPG | | | | | | | | RQAPGKGLEWM | | | | | | G | |
| | KAPKLMIYGV | | | | | | | | GIIDPGDSRTRYS | | | | | | | |
| | NNRPSGVSNR | | | | | | | | PSFQGQVTISADK | | | | | | | |
| | FSGSKSGNTAS | | | | | | | | SISTAYLQWSSLK | | | | | | | |
| | LTISGLQAEDE | | | | | | | | ASDTAMYYCAR | | | | | | | |
| | ADYYCSSYDI | | | | | | | | GQLYGGTYMDG | | | | | | | |
| | ESATPVFGGG | | | | | | | | WGQGTLVTVSS | | | | | | | |
| | TKLTVL | | | | | | | | | | | | | | | |
| Meso- | DIELTQSPAIM | 1593 | SSVS | 1594 | DTS | 1595 | QQW | 1596 | QVQLQQSGPELE | 1597 | GYSF | 1598 | ITPY | 1599 | ARGG | 1600 |
| thelin | SASPGEKVTM | | Y | | | | SKHP | | KPGASVKISCKA | | TGY | | NGAS | | YDGR | |
| | TCSASSSVSY | | | | | | LT | | SGYSFTGYTMN | | T | | | | GFDY | |
| | MHWYQQKSG | | | | | | | | WVKQSHGKSLE | | | | | | | |
| | TSPKRWIYDTS | | | | | | | | WIGLITPYNGASS | | | | | | | |
| | KLASGVPGRF | | | | | | | | YNQKFRGKATLT | | | | | | | |
| | SGSGSGNSYSL | | | | | | | | VDKSSSTAYMDL | | | | | | | |
| | TISSVEAEDDA | | | | | | | | LSLTSEDSAVYFC | | | | | | | |
| | TYYCQQWSK | | | | | | | | ARGGYDGRGFD | | | | | | | |
| | HPLTFGSGTK | | | | | | | | YWGSGTPVTVSS | | | | | | | |
| | VEIK | | | | | | | | | | | | | | | |
| MT1- | DIQMTQSPSSL | 1601 | QDVR | 1602 | SSS | 1603 | QQH | 1604 | QVQLQESGPGLV | 1605 | GFSL | 1606 | IWTG | 1607 | ARYY | 1608 |
| MMP | SASVGDRVTIT | | NT | | | | YITP | | KPSETLSLTCTVS | | LSYG | | GTT | | YGMD | |
| (MMP14) | CKASQDVRNT | | | | | | YT | | GFSLLSYGVHWV | | | | | | Y | |
| | VAWYQQKPG | | | | | | | | RQPPGKGLEWLG | | | | | | | |
| | KAPKLLIYSSS | | | | | | | | VIWTGGTTNYNS | | | | | | | |
| | YRNTGVPDRF | | | | | | | | ALMSRFTISKDDS | | | | | | | |
| | SGSGSGTDFTL | | | | | | | | KNTVYLKMNSL | | | | | | | |
| | TISSLQAEDVA | | | | | | | | KTEDTAIYYCAR | | | | | | | |
| | VYYCQQHYIT | | | | | | | | YYYGMDYWGQ | | | | | | | |
| | PYTFGGGTKV | | | | | | | | GTLVTVSS | | | | | | | |
| | EIK | | | | | | | | | | | | | | | |
| MUC1 | DIQLTQSPSSL | 1609 | SSVS | 1610 | STS | 1611 | HQW | 1612 | QVQLQQSGAEV | 1613 | GYTF | 1614 | INPY | 1615 | ARGF | 1616 |
| | SASVGDRVTM | | SSY | | | | NRYP | | KKPGASVKVSCE | | PSYV | | NDGT | | GGSY | |
| | TCSASSSVSSS | | | | | | YT | | ASGYTFPSYVLH | | | | | | GFAY | |
| | YLYWYQQKP | | | | | | | | WVKQAPGQGLE | | | | | | | |
| | GKAPKLWIYS | | | | | | | | WIGYINPYNDGT | | | | | | | |
| | TSNLASGVPA | | | | | | | | QYNEKFKGKATL | | | | | | | |
| | RFSGSGSGTDF | | | | | | | | TRDTSINTAYME | | | | | | | |
| | TLTISSLQPED | | | | | | | | LSRLRSDDTAVY | | | | | | | |
| | SASYFCHQWN | | | | | | | | YCARGFGGSYGF | | | | | | | |
| | RYPYTFGGGT | | | | | | | | AYWGQGTLVTV | | | | | | | |
| | RLEIK | | | | | | | | SS | | | | | | | |
| Mucin | QVVLTQSPVI | 1617 | SSIS | 1618 | DTS | 1619 | HQR | 1620 | QVQLKESGPDLV | 1621 | GFSL | 1622 | IWGD | 1623 | VKPG | 1624 |
| 5AC | MSASPGEKVT | | Y | | | | DSYP | | APSQSLSITCTVS | | SKFG | | GST | | GDY | |
| | MTCSASSSISY | | | | | | WT | | GFSLSKFGVNWV | | | | | | | |
| | MYWYQQKPG | | | | | | | | RQPPGKGLEWLG | | | | | | | |
| | TSPKRWIYDTS | | | | | | | | VIWGDGSTSYNS | | | | | | | |
| | KLASGVPARF | | | | | | | | GLISRLSISKENS | | | | | | | |
| | SGSGSGTSYSL | | | | | | | | KSQVFLKLNSLQ | | | | | | | |
| | TISNMEAGDA | | | | | | | | ADDTATYYCVKP | | | | | | | |
| | ATYYCHQRDS | | | | | | | | GGDYWGHGTSV | | | | | | | |
| | YPWTFGGGIN | | | | | | | | TVSS | | | | | | | |
| | LEIK | | | | | | | | | | | | | | | |
| NaPi2b | DIQMTQSPSSL | 1625 | ETLV | 1626 | RVS | 1627 | FQGS | 1628 | EVQLVESGGGLV | 1629 | GFSF | 1630 | IGRV | 1631 | ARHR | 1632 |
| | SASVGDRVTIT | | HSSG | | | | FNPL | | QPGGSLRLSCAA | | SDFA | | AFHT | | GFDV | |
| | CRSSETLVHSS | | NTY | | | | T | | SGFSFSDFAMSW | | | | | | GHFD | |
| | GNTYLEWYQ | | | | | | | | VRQAPGKGLEW | | | | | | F | |
| | QKPGKAPKLLI | | | | | | | | VATIGRVAFHTY | | | | | | | |
| | YRVSNRFSGV | | | | | | | | YPDSMKGRFTIS | | | | | | | |
| | PSRFSGSGSGT | | | | | | | | RDNSKNTLYLQ | | | | | | | |
| | DFTLTISSLQP | | | | | | | | MNSLRAEDTAV | | | | | | | |
| | EDFATYYCFQ | | | | | | | | YYCARHRGFDV | | | | | | | |
| | GSFNPLTFGQ | | | | | | | | GHFDFWGQGTL | | | | | | | |
| | GTKVEIK | | | | | | | | VTVSS | | | | | | | |
| NeuGc- | DIVMTQSHKF | 1633 | QDVS | 1634 | SAS | 1635 | QQH | 1636 | EVQLKESGPGLV | 1637 | GFSL | 1638 | IWGG | 1639 | ARSG | 1640 |
| GM3 | MSTSVGDRVS | | TA | | | | YSTP | | APSQSLSITCTVS | | SRYS | | GST | | VREG | |
| | ITCKASQDVST | | | | | | WT | | GFSLSRYSVHWV | | | | | | RAQA | |
| | AVAWYQQKP | | | | | | | | RQPPGKGLEWLG | | | | | | WFAY | |
| | GQSPKLLIYSA | | | | | | | | MIWGGGSTDYNS | | | | | | | |
| | SYRYTGVPDR | | | | | | | | ALKSRLSISKDNS | | | | | | | |
| | FTGSGSGTDFT | | | | | | | | KSQVFLKMNSLQ | | | | | | | |
| | FTISSVQAEDL | | | | | | | | TDDTAMYYCAR | | | | | | | |
| | AVYYCQQHYS | | | | | | | | SGVREGRAQAW | | | | | | | |
| | TPWTFGGGTK | | | | | | | | FAYWGQGTLVT | | | | | | | |
| | LELK | | | | | | | | VSA | | | | | | | |
| NKG2A | DIQMTQSPSSL | 1641 | ENIY | 1642 | NAK | 1643 | QHH | 1644 | QVQLVQSGAEV | 1645 | GYTF | 1646 | IDPY | 1647 | ARGG | 1648 |
| | SASVGDRVTIT | | SY | | | | YGTP | | KKPGASVKVSCK | | TSY | | DSET | | YDFD | |
| | CRASENIYSYL | | | | | | RT | | ASGYTFTSYWM | | W | | | | VGTL | |
| | AWYQQKPGK | | | | | | | | NWVRQAPGQGL | | | | | | YWFF | |
| | APKLLIYNAK | | | | | | | | EWMGRIDPYDSE | | | | | | DV | |
| | TLAEGVPSRFS | | | | | | | | THYAQKLQGRV | | | | | | | |
| | GSGSGTDFTLT | | | | | | | | TMTTDTSTSTAY | | | | | | | |
| | ISSLQPEDFAT | | | | | | | | MELRSLRSDDTA | | | | | | | |
| | YYCQHHYGTP | | | | | | | | VYYCARGGYDF | | | | | | | |
| | RTFGGGTKVEI | | | | | | | | DVGTLYWFFDV | | | | | | | |
| | K | | | | | | | | WGQGTTVTVSS | | | | | | | |
| notch | QAVVTQEPSL | 1649 | TGAV | 1650 | GTN | 1651 | ALW | 1652 | QVQLVQSGAEV | 1653 | GAS | 1654 | ILPG | 1655 | ARFD | 1656 |
| | TVSPGGTVTL | | TTSN | | | | YSN | | KKPGASVKISCK | | VKIS | | TGRT | | GNYG | |
| | TCRSSTGAVT | | Y | | | | HWV | | VSGYTLRGYWIE | | CKV | | | | YYAM | |
| | TSNYANWFQQ | | | | | | | | WVRQAPGKGLE | | S | | | | DYW | |
| | KPGQAPRTLIG | | | | | | | | WIGQILPGTGRT | | | | | | | |
| | GTNNRAPGVP | | | | | | | | NYNEKFKGRVT | | | | | | | |
| | ARFSGSLLGG | | | | | | | | MTADTSTDTAY | | | | | | | |
| | KAALTLSGAQ | | | | | | | | MELSSLRSEDTA | | | | | | | |
| | PEDEAEYYCA | | | | | | | | VYYCARFDGNY | | | | | | | |
| | LWYSNHWVF | | | | | | | | GYYAMDYWGQ | | | | | | | |
| | GGGTKLTV | | | | | | | | GTTVTVSS | | | | | | | |
| NOTCH2/ | DIVLTQSPATL | 1657 | QSVR | 1658 | GAS | 1659 | QQY | 1660 | EVQLVESGGGLV | 1661 | GFTF | 1662 | IASS | 1663 | ARSI | 1664 |
| NOTCH3 | SLSPGERATLS | | SNY | | | | SNFP | | QPGGSLRLSCAA | | SSSG | | GSNT | | FYTT | |
| recep- | CRASQSVRSN | | | | | | IT | | SGFTFSSSGMSW | | | | | | | |
| tors | YLAWYQQKP | | | | | | | | VRQAPGKGLEW | | | | | | | |
| | GQAPRLLIYG | | | | | | | | VSVIASSGSNTYY | | | | | | | |
| | ASSRATGVPA | | | | | | | | ADSVKGRFTISR | | | | | | | |
| | RFSGSGSGTDF | | | | | | | | DNSKNTLYLQM | | | | | | | |
| | TLTISSLEPEDF | | | | | | | | NSLRAEDTAVYY | | | | | | | |
| | AVYYCQQYSN | | | | | | | | CARSIFYTTWGQ | | | | | | | |
| | FPITFGQGTKV | | | | | | | | GTLVTVSS | | | | | | | |
| | EIK | | | | | | | | | | | | | | | |
| NRP1 | DIQMTQSPSSL | 1665 | QYFS | 1666 | GAS | 1667 | QQY | 1668 | EVQLVESGGGLV | 1669 | GFTF | 1670 | ISPA | 1671 | ARGE | 1672 |
| | SASVGDRVTIT | | SY | | | | LGSP | | QPGGSLRLSCAA | | SSYA | | GGYT | | LPYY | |
| | CRASQYFSSY | | | | | | PT | | SGFTFSSYAMSW | | | | | | RMSK | |
| | LAWYQQKPG | | | | | | | | VRQAPGKGLEW | | | | | | VMDV | |
| | KAPKLLIYGAS | | | | | | | | VSQISPAGGYTN | | | | | | | |
| | SRASGVPSRFS | | | | | | | | YADSVKGRFTIS | | | | | | | |
| | GSGSGTDFTLT | | | | | | | | ADTSKNTAYLQ | | | | | | | |
| | ISSLQPEDFAT | | | | | | | | MNSLRAEDTAV | | | | | | | |
| | YYCQQYLGSP | | | | | | | | YYCARGELPYYR | | | | | | | |
| | PTFGQGTKVEI | | | | | | | | MSKVMDVWGQ | | | | | | | |
| | K | | | | | | | | GTLVTVSS | | | | | | | |
| oxLDL | QSVLTQPPSAS | 1673 | NTNI | 1674 | ANS | 1675 | ASW | 1676 | EVQLLESGGGLV | 1677 | GFTF | 1678 | ISVG | 1679 | ARIR | 1680 |
| | GTPGQRVTISC | | GKNY | | | | DASL | | QPGGSLRLSCAA | | SNA | | GHRT | | VGPS | |
| | SGSNTNIGKN | | | | | | NGW | | SGFTFSNAWMS | | W | | | | GGAF | |
| | YVSWYQQLPG | | | | | | V | | WVRQAPGKGLE | | | | | | DY | |
| | TAPKLLIYANS | | | | | | | | WVSSISVGGHRT | | | | | | | |
| | NRPSGVPDRFS | | | | | | | | YYADSVKGRSTI | | | | | | | |
| | GSKSGTSASL | | | | | | | | SRDNSKNTLYLQ | | | | | | | |
| | AISGLRSEDEA | | | | | | | | MNSLRAEDTAV | | | | | | | |
| | DYYCASWDA | | | | | | | | YYCARIRVGPSG | | | | | | | |
| | SLNGWVFGGG | | | | | | | | GAFDYWGQGTL | | | | | | | |
| | TKLTVL | | | | | | | | VTVSS | | | | | | | |
| P- | EIVLTQSPATL | 1681 | QSVS | 1682 | DAS | 1683 | QQRS | 1684 | EVQLVESGGGLV | 1685 | GFTF | 1686 | ITAA | 1687 | ARGR | 1688 |
| selectin | SLSPGERATLS | | SY | | | | NWP | | RPGGSLRLSCAA | | SNY | | GDI | | YSGS | |
| | CRASQSVSSY | | | | | | LT | | SGFTFSNYDMH | | D | | | | GSYY | |
| | LAWYQQKPG | | | | | | | | WVRQATGKGLE | | | | | | NDWF | |
| | QAPRLLIYDAS | | | | | | | | WVSAITAAGDIY | | | | | | DP | |
| | NRATGIPARFS | | | | | | | | YPGSVKGRFTISR | | | | | | | |
| | GSGSGTDFTLT | | | | | | | | ENAKNSLYLQM | | | | | | | |
| | ISSLEPEDFAV | | | | | | | | NSLRAGDTAVYY | | | | | | | |
| | YYCQQRSNWP | | | | | | | | CARGRYSGSGSY | | | | | | | |
| | LTFGGGTKVEI | | | | | | | | YNDWFDPWGQG | | | | | | | |
| | K | | | | | | | | TLVTVSS | | | | | | | |
| PCSK9 | DIVMTQSPDSL | 1689 | QSVL | 1690 | WAS | 1691 | QQY | 1692 | EVQLVESGGGLV | 1693 | GFTF | 1694 | ISGS | 1695 | AKDS | 1696 |
| | AVSLGERATIN | | YRSN | | | | YTTP | | QPGGSLRLSCAA | | NNY | | GGTT | | NWGN | |
| | CKSSQSVLYR | | NRNF | | | | YT | | SGFTFNNYAMN | | A | | | | FDL | |
| | SNNRNFLGWY | | | | | | | | WVRQAPGKGLD | | | | | | | |
| | QQKPGQPPNL | | | | | | | | WVSTISGSGGTT | | | | | | | |
| | LIYWASTRES | | | | | | | | NYADSVKGRFIIS | | | | | | | |
| | GVPDRFSGSG | | | | | | | | RDSSKHTLYLQM | | | | | | | |
| | SGTDFTLTISS | | | | | | | | NSLRAEDTAVYY | | | | | | | |
| | LQAEDVAVYY | | | | | | | | CAKDSNWGNFD | | | | | | | |
| | CQQYYTTPYT | | | | | | | | LWGRGTLVTVSS | | | | | | | |
| | FGQGTKLEIK | | | | | | | | | | | | | | | |
| PCSK9 | ESALTQPASVS | 1697 | SSDV | 1698 | EVS | 1699 | NSYT | 1700 | EVQLVQSGAEVK | 1701 | GYT | 1702 | VSFY | 1703 | ARGY | 1704 |
| | GSPGQSITISCT | | GGYN | | | | STSM | | KPGASVKVSCKA | | LTSY | | NGNT | | GMDV | |
| | GTSSDVGGYN | | S | | | | V | | SGYTLTSYGISW | | G | | | | | |
| | SVSWYQQHPG | | | | | | | | VRQAPGQGLEW | | | | | | | |
| | KAPKLMIYEV | | | | | | | | MGWVSFYNGNT | | | | | | | |
| | SNRPSGVSNRF | | | | | | | | NYAQKLQGRGT | | | | | | | |
| | SGSKSGNTAS | | | | | | | | MTTDPSTSTAYM | | | | | | | |
| | LTISGLQAEDE | | | | | | | | ELRSLRSDDTAV | | | | | | | |
| | ADYYCNSYTS | | | | | | | | YYCARGYGMDV | | | | | | | |
| | TSMVFGGGTK | | | | | | | | WGQGTTVTVSS | | | | | | | |
| | LTVL | | | | | | | | | | | | | | | |
| PCSK9 | DIQMTQSPSSL | 1705 | QGIS | 1706 | SAS | 1707 | QQR | 1708 | QVQLVQSGAEV | 1709 | GYTF | 1710 | ISPF | 1711 | ARER | 1712 |
| | SASVGDRVTIT | | SA | | | | YSL | | KKPGASVKVSCK | | TSYY | | GGRT | | PLYA | |
| | CRASQGISSAL | | | | | | WRT | | ASGYTFTSYYMH | | | | | | SDL | |
| | AWYQQKPGK | | | | | | | | WVRQAPGQGLE | | | | | | | |
| | APKLLIYSASY | | | | | | | | WMGEISPFGGRT | | | | | | | |
| | RYTGVPSRFS | | | | | | | | NYNEKFKSRVTM | | | | | | | |
| | GSGSGTDFTFT | | | | | | | | TRDTSTSTVYME | | | | | | | |
| | ISSLQPEDIAT | | | | | | | | LSSLRSEDTAVY | | | | | | | |
| | YYCQQRYSL | | | | | | | | YCARERPLYASD | | | | | | | |
| | WRTFGQGTKL | | | | | | | | LWGQGTTVTVSS | | | | | | | |
| | EIK | | | | | | | | | | | | | | | |
| PDGFR | EIVLTQSPATL | 1713 | QSVS | 1714 | DAS | 1715 | QQRS | 1716 | QLQLQESGPGLV | 1717 | GGSI | 1718 | FFYT | 1719 | ARQS | 1720 |
| A | SLSPGERATLS | | SY | | | | NWP | | KPSETLSLTCTVS | | NSSS | | GST | | TYYY | |
| | CRASQSVSSY | | | | | | PA | | GGSINSSSYYWG | | YY | | | | GSGN | |
| | LAWYQQKPG | | | | | | | | WLRQSPGKGLE | | | | | | YYGW | |
| | QAPRLLIYDAS | | | | | | | | WIGSFFYTGSTY | | | | | | FDR | |
| | NRATGIPARFS | | | | | | | | YNPSLRSRLTISV | | | | | | | |
| | GSGSGTDFTLT | | | | | | | | DTSKNQFSLMLS | | | | | | | |
| | ISSLEPEDFAV | | | | | | | | SVTAADTAVYYC | | | | | | | |
| | YYCQQRSNWP | | | | | | | | ARQSTYYYGSGN | | | | | | | |
| | PAFGQGTKVEI | | | | | | | | YYGWFDRWDQG | | | | | | | |
| | K | | | | | | | | TLVTVSS | | | | | | | |
| PDGFRa | DIQMTQSPSSL | 1721 | QSFS | 1722 | AAS | 1723 | QQT | 1724 | QVQLVESGGGLV | 1725 | GFTF | 1726 | ISSS | 1727 | AREG | 1728 |
| | SASVGDRVSIT | | RY | | | | YSNP | | KPGGSLRLSCAA | | SDY | | GSII | | RIAA | |
| | CRPSQSFSRYI | | | | | | PIT | | SGFTFSDYYMN | | Y | | | | RGMD | |
| | NWYQQKPGK | | | | | | | | WIRQAPGKGLE | | | | | | V | |
| | APKLLIHAASS | | | | | | | | WVSYISSSGSIIY | | | | | | | |
| | LVGGVPSRFS | | | | | | | | YADSVKGRFTIS | | | | | | | |
| | GSGSGTDFTLT | | | | | | | | RDNAKNSLYLQ | | | | | | | |
| | ISSLQPEDFAT | | | | | | | | MNSLRAEDTAV | | | | | | | |
| | YYCQQTYSNP | | | | | | | | YYCAREGRIAAR | | | | | | | |
| | PITFGQGTRLE | | | | | | | | GMDVWGQGTTV | | | | | | | |
| | MK | | | | | | | | TVSS | | | | | | | |
| phospha- | DIQMTQSPSSL | 1729 | QDIG | 1730 | ATS | 1731 | LQY | 1732 | EVQLQQSGPELE | 1733 | GYSF | 1734 | IDPY 1735 | VKGG | 1736 | |
| tidyl- | SASLGERVSLT | | SS | | | | VSSP | | KPGASVKLSCKA | | TGY | | Y | GDT | | YYGH |
| serine | CRASQDIGSSL | | | | | | PT | | SGYSFTGYNMN | | N | | | | WYFD | |
| | NWLQQGPDG | | | | | | | | WVKQSHGKSLE | | | | | | V | |
| | TIKRLIYATSS | | | | | | | | WIGHIDPYYGDT | | | | | | | |
| | LDSGVPKRFS | | | | | | | | SYNQKFRGKATL | | | | | | | |
| | GSRSGSDYSLT | | | | | | | | TVDKSSSTAYMQ | | | | | | | |
| | ISSLESEDFVD | | | | | | | | LKSLTSEDSAVY | | | | | | | |
| | YYCLQYVSSP | | | | | | | | YCVKGGYYGHW | | | | | | | |
| | PTFGAGTKLE | | | | | | | | YFDVWGAGTTV | | | | | | | |
| | LK | | | | | | | | TVSS | | | | | | | |
| polysi- | DVVMTQTPLS | 1737 | QSLV | 1738 | RVS | 1739 | FQGT | 1740 | QIQLQQSGPELV | 1741 | GYTF | 1742 | IYPG | 1743 | ARGG | 1744 |
| alic | LPVSLGDQASI | | HSNG | | | | HVP | | RPGASVKISCKAS | | TDY | | SGNT | | KFAM | |
| acid | SCRSSQSLVHS | | NTY | | | | YT | | GYTFTDYYIHWV | | Y | | | | DY | |
| | NGNTYLYWY | | | | | | | | KQRPGEGLEWIG | | | | | | | |
| | LQKPGQSPKP | | | | | | | | WIYPGSGNTKYN | | | | | | | |
| | LIYRVSNRFSG | | | | | | | | EKFKGKATLTVD | | | | | | | |
| | VPDRFSGSGS | | | | | | | | TSSSTAYMQLSS | | | | | | | |
| | GTDFTLKISRV | | | | | | | | LTSEDSAVYFCA | | | | | | | |
| | EAEDLGVYFC | | | | | | | | RGGKFAMDYWG | | | | | | | |
| | FQGTHVPYTF | | | | | | | | QGTSVTVSS | | | | | | | |
| | GGGTRLEIK | | | | | | | | | | | | | | | |
| PSMA | DIVMTQSHKF | 1745 | QDVG | 1746 | WAS | 1747 | QQY | 1748 | EVQLQQSGPELV | 1749 | GYTF | 1750 | INPN | 1751 | AAGW | 1752 |
| | MSTSVGDRVS | | TA | | | | NSYP | | KPGTSVRISCKTS | | TEYT | | NGGT | | NFDY | |
| | IICKASQDVGT | | | | | | LTFG | | GYTFTEYTIHWV | | | | | | | |
| | AVDWYQQKP | | | | | | AGT | | KQSHGKSLEWIG | | | | | | | |
| | GQSPKLLIYW | | | | | | M | | NINPNNGGTTYN | | | | | | | |
| | ASTRHTGVPD | | | | | | | | QKFEDKATLTVD | | | | | | | |
| | RFTGSGSGTDF | | | | | | | | KSSSTAYMELRS | | | | | | | |
| | TLTITNVQSED | | | | | | | | LTSEDSAVYYCA | | | | | | | |
| | LADYFCQQYN | | | | | | | | AGWNFDYWGQG | | | | | | | |
| | SYPLTFGAGT | | | | | | | | TTLTVSS | | | | | | | |
| | MLDLK | | | | | | | | | | | | | | | |
| PSMA | DIQMTQSPSSL | 1753 | QNVD | 1754 | SAS | 1755 | QQY | 1756 | QVQLVESGGGLV | 1757 | GFTF | 1758 | ISDG | 1759 | ARGF | 1760 |
| | SASVGDRVTIT | | TN | | | | DSYP | | KPGESLRLSCAA | | SDY | | GYYT | | PLLR | |
| | CKASQNVDTN | | | | | | YT | | SGFTFSDYYMY | | Y | | | | HGAM | |
| | VAWYQQKPG | | | | | | | | WVRQAPGKGLE | | | | | | DY | |
| | QAPKSLIYSAS | | | | | | | | WVAIISDGGYYT | | | | | | | |
| | YRYSDVPSRFS | | | | | | | | YYSDIIKGRFTISR | | | | | | | |
| | GSASGTDFTLT | | | | | | | | DNAKNSLYLQM | | | | | | | |
| | ISSVQSEDFAT | | | | | | | | NSLKAEDTAVYY | | | | | | | |
| | YYCQQYDSYP | | | | | | | | CARGFPLLRHGA | | | | | | | |
| | YTFGGGTKLEI | | | | | | | | MDYWGQGTLVT | | | | | | | |
| | K | | | | | | | | VSS | | | | | | | |
| PVRL4 | DIQMTQSPSSV | 1761 | QGIS | 1762 | AAS | 1763 | QQA | 1764 | EVQLVESGGGLV | 1765 | GFTF | 1766 | ISSS | 1767 | ARAY | 1768 |
| | SASVGDRVTIT | | GW | | | | NSFP | | QPGGSLRLSCAA | | SSYN | | SSTI | | YYGM | |
| | CRASQGISGW | | | | | | PT | | SGFTFSSYNMNW | | | | | | DV | |
| | LAWYQQKPG | | | | | | | | VRQAPGKGLEW | | | | | | | |
| | KAPKFLIYAAS | | | | | | | | VSYISSSSSTIYYA | | | | | | | |
| | TLQSGVPSRFS | | | | | | | | DSVKGRFTISRD | | | | | | | |
| | GSGSGTDFTLT | | | | | | | | NAKNSLSLQMNS | | | | | | | |
| | ISSLQPEDFAT | | | | | | | | LRDEDTAVYYCA | | | | | | | |
| | YYCQQANSFP | | | | | | | | RAYYYGMDVW | | | | | | | |
| | PTFGGGTKVEI | | | | | | | | GQGTTVTVSS | | | | | | | |
| | K | | | | | | | | | | | | | | | |
| RGMA | QSALTQPRSVS | 1769 | SSSV | 1770 | DVT | 1771 | YSY | 1772 | EVQLVQSGAEVK | 1773 | GYTF | 1774 | ISPY | 1775 | ARVG | 1776 |
| | GSPGQSVTISC | | GDSI | | | | AGT | | KPGASVKVSCKA | | TSHG | | SGNT | | SGPY | |
| | TGTSSSVGDSI | | Y | | | | DTL | | SGYTFTSHGISW | | | | | | YYMD | |
| | YVSWYQQHP | | | | | | | | VRQAPGQGLDW | | | | | | V | |
| | GKAPKLMLYD | | | | | | | | MGWISPYSGNTN | | | | | | | |
| | VTKRPSGVPD | | | | | | | | YAQKLQGRVTM | | | | | | | |
| | RFSGSKSGNT | | | | | | | | TTDTSTSTAYME | | | | | | | |
| | ASLTISGLQAE | | | | | | | | LSSLRSEDTAVY | | | | | | | |
| | DEADYYCYSY | | | | | | | | YCARVGSGPYYY | | | | | | | |
| | AGTDTLFGGG | | | | | | | | MDVWGQGTLVT | | | | | | | |
| | TKVTVL | | | | | | | | VSS | | | | | | | |
| CD240D | AIRMTQSPSSF | 1777 | QDIR | 1778 | AAS | 1779 | QQY | 1780 | QVQLVESGGGV | 1781 | GFTF | 1782 | ISYD | 1783 | ARPV | 1784 |
| Blood | SASTGDRVTIT | | NY | | | | YNSP | | VQPGRSLRLSCT | | KNY | | GRNI | | RSRW | |
| group D | CRASQDIRNY | | | | | | PT | | ASGFTFKNYAMEI | | A | | | | LQLG | |
| antigen | VAWYQQKSG | | | | | | | | WVRQAPAKGLE | | | | | | LEDA | |
| | KAPKFLIYAAS | | | | | | | | WVATISYDGRNI | | | | | | FHI | |
| | TLQSGVPSRFS | | | | | | | | QYADSVKGRFTF | | | | | | | |
| | GSGSGTDFTLT | | | | | | | | SRDNSQDTLYLQ | | | | | | | |
| | INSLQSEDFAT | | | | | | | | LNSLRPEDTAVY | | | | | | | |
| | YYCQQYYNSP | | | | | | | | YCARPVRSRWLQ | | | | | | | |
| | PTFGQGTRVEI | | | | | | | | LGLEDAFHIWGQ | | | | | | | |
| | T | | | | | | | | GTMVTVSS | | | | | | | |
| root | DIQMTQSPSSL | 1785 | QSVD | 1786 | AAS | 1787 | QQS | 1788 | QVQLVQSGAEV | 1789 | GYTF | 1790 | IYPS | 1791 | ATYF | 1792 |
| plate- | SASVGDRVTIT | | YDGD | | | | NEDP | | KKPGASVKVSCK | | TDYS | | NGDS | | ANNF | |
| speci- | CKASQSVDYD | | SY | | | | LT | | ASGYTFTDYSIH | | | | | | DYW | |
| fic | GDSYMNWYQ | | | | | | | | WVRQAPGQGLE | | | | | | | |
| spondin | QKPGKAPKLLI | | | | | | | | WIGYIYPSNGDS | | | | | | | |
| 3 | YAASNLESGV | | | | | | | | GYNQKFKNRVT | | | | | | | |
| | PSRFSGSGSGT | | | | | | | | MTRDTSTSTAYM | | | | | | | |
| | DFTLTISPVQA | | | | | | | | ELSRLRSEDTAV | | | | | | | |
| | EDFATYYCQQ | | | | | | | | YYCATYFANNFD | | | | | | | |
| | SNEDPLTFGA | | | | | | | | YWGQGTTLTVSS | | | | | | | |
| | GTKLELK | | | | | | | | | | | | | | | |
| serum | DIQMTQSPSSL | 1793 | ENIY | 1794 | NAK | 1795 | QHH | 1796 | QVQLVQSGAEV | 1797 | GFTF | 1798 | IYPG | 1799 | ARGD | 1800 |
| amyloid | SASVGDRVTIT | | SY | | | | YGA | | KKPGSSVKVSCK | | ATY | | DGNA | | FDYD | |
| P | CRASENIYSYL | | | | | | PLT | | ASGFTFATYNMH | | N | | | | GGYY | |
| com- | AWYQQKPGK | | | | | | | | WVRQAPGQGLE | | | | | | FDS | |
| ponent | APKLLIHNAK | | | | | | | | WMGYIYPGDGN | | | | | | | |
| | TLAEGVPSRFS | | | | | | | | ANYNQQFKGRV | | | | | | | |
| | GSGSGTDFTLT | | | | | | | | TITADKSTSTAY | | | | | | | |
| | ISSLQPEDFAT | | | | | | | | MELSSLRSEDTA | | | | | | | |
| | YYCQHHYGAP | | | | | | | | VYYCARGDFDY | | | | | | | |
| | LTFGQGTKLEI | | | | | | | | DGGYYFDSWGQ | | | | | | | |
| | K | | | | | | | | GTLVTVSS | | | | | | | |
| STEAP- | DIQMTQSPSSL | 1801 | QSLL | 1802 | WAS | 1803 | QQY | 1804 | EVQLVESGGGLV | 1805 | GYSI | 1806 | ISNS | 1807 | ARER | 1808 |
| 1 | SASVGDRVTIT | | YRSN | | | | YNY | | QPGGSLRLSCAV | | TSDY | | GST | | NYDY | |
| | CKSSQSLLYRS | | QKNY | | | | PRT | | SGYSITSDYAWN | | A | | | | DDYY | |
| | NQKNYLAWY | | | | | | | | WVRQAPGKGLE | | | | | | YAMD | |
| | QQKPGKAPKL | | | | | | | | WVGYISNSGSTS | | | | | | Y | |
| | LIYWASTRES | | | | | | | | YNPSLKSRFTISR | | | | | | | |
| | GVPSRFSGSGS | | | | | | | | DTSKNTLYLQMN | | | | | | | |
| | GTDFTLTISSL | | | | | | | | SLRAEDTAVYYC | | | | | | | |
| | QPEDFATYYC | | | | | | | | ARERNYDYDDY | | | | | | | |
| | QQYYNYPRTF | | | | | | | | YYAMDYWGQGT | | | | | | | |
| | GQGTKVEIK | | | | | | | | LVTVSS | | | | | | | |
| TACST | DIQLTQSPSSL | 1809 | QDVS | 1810 | SAS | 1811 | QQH | 1812 | QVQLQQSGSELK | 1813 | GYTF | 1814 | INTY | 1815 | ARGG | 1816 |
| D2 | SASVGDRVSIT | | IA | | | | YITP | | KPGASVKVSCKA | | TNY | | TGEP | | FGSS | |
| | CKASQDVSIA | | | | | | LT | | SGYTFTNYGMN | | G | | | | YWYF | |
| | VAWYQQKPG | | | | | | | | WVKQAPGQGLK | | | | | | DV | |
| | KAPKLLIYSAS | | | | | | | | WMGWINTYTGE | | | | | | | |
| | YRYTGVPDRF | | | | | | | | PTYTDDFKGRFA | | | | | | | |
| | SGSGSGTDFTL | | | | | | | | FSLDTSVSTAYL | | | | | | | |
| | TISSLQPEDFA | | | | | | | | QISSLKADDTAV | | | | | | | |
| | VYYCQQHYIT | | | | | | | | YFCARGGFGSSY | | | | | | | |
| | PLTFGAGTKV | | | | | | | | WYFDVWGQGSL | | | | | | | |
| | EIK | | | | | | | | VTVSS | | | | | | | |
| TGFb | ETVLTQSPGTL | 1817 | QSLG | 1818 | GAS | 1819 | QQY | 1820 | QVQLVQSGAEV | 1821 | GYTF | 1822 | VIPI | 1823 | ASTL | 1824 |
| | SLSPGERATLS | | SSY | | | | ADSP | | KKPGSSVKVSCK | | SSNV | | VDIA | | GLVL | |
| | CRASQSLGSS | | | | | | IT | | ASGYTFSSNVIS | | | | | | DAMD | |
| | YLAWYQQKP | | | | | | | | WVRQAPGQGLE | | | | | | Y | |
| | GQAPRLLIYG | | | | | | | | WMGGVIPIVDIA | | | | | | | |
| | ASSRAPGIPDR | | | | | | | | NYAQRFKGRVTI | | | | | | | |
| | FSGSGSGTDFT | | | | | | | | TADESTSTTYME | | | | | | | |
| | LTISRLEPEDF | | | | | | | | LSSLRSEDTAVY | | | | | | | |
| | AVYYCQQYA | | | | | | | | YCASTLGLVLDA | | | | | | | |
| | DSPITFGQGTR | | | | | | | | MDYWGQGTLVT | | | | | | | |
| | LEIK | | | | | | | | VSS | | | | | | | |
| TIGIT | DIVMTQSPDSL | 1825 | QTVL | 1826 | WAS | 1827 | QQY | 1828 | EVQLQQSGPGLV | 1829 | GDS | 1830 | TYYR | 1831 | TRES | 1832 |
| | AVSLGERATIN | | YSSN | | | | YSTP | | KPSQTLSLTCAIS | | VSSN | | FKWY | | TTYD | |
| | CKSSQTVLYSS | | NKKY | | | | FT | | GDSVSSNSAAWN | | SAA | | S | | LLAG | |
| | NNKKYLAWY | | | | | | | | WIRQSPSRGLEW | | | | | | PFDY | |
| | QQKPGQPPNL | | | | | | | | LGKTYYRFKWY | | | | | | | |
| | LIYWASTRES | | | | | | | | SDYAVSVKGRITI | | | | | | | |
| | GVPDRFSGSG | | | | | | | | NPDTSKNQFSLQ | | | | | | | |
| | SGTDFTLTISS | | | | | | | | LNSVTPEDTAVF | | | | | | | |
| | LQAEDVAVYY | | | | | | | | YCTRESTTYDLL | | | | | | | |
| | CQQYYSTPFTF | | | | | | | | AGPFDYWGQGT | | | | | | | |
| | GPGTKVEIK | | | | | | | | LVTVSS | | | | | | | |
| TWEAK | DIQMTQSPSSL | 1833 | QSVS | 1834 | YAS | 1835 | QHS | 1836 | EVQLVESGGGLV | 1837 | GFTF | 1838 | IRLK | 1839 | TGYY | 1840 |
| R | SASVGDRVTIT | | TSSY | | | | WEIP | | QPGGSLRLSCAA | | SSY | | SDNY | | ADAM | |
| | CRASQSVSTSS | | SY | | | | YT | | SGFTFSSYWMSW | | W | | AT | | DY | |
| | YSYMHWYQQ | | | | | | | | VRQAPGKGLEW | | | | | | | |
| | KPGKAPKLLIK | | | | | | | | VAEIRLKSDNYA | | | | | | | |
| | YASNLESGVP | | | | | | | | THYAESVKGRFT | | | | | | | |
| | SRFSGSGSGTD | | | | | | | | ISRDDSKNSLYLQ | | | | | | | |
| | FTLTISSLQPE | | | | | | | | MNSLRAEDTAV | | | | | | | |
| | DFATYYCQHS | | | | | | | | YYCTGYYADAM | | | | | | | |
| | WEIPYTFGGG | | | | | | | | DYWGQGTLVTV | | | | | | | |
| | TKVEIK | | | | | | | | SS | | | | | | | |
| TYRP1 | EIVLTQSPATL | 1841 | QSVS | 1842 | DAS | 1843 | QQRS | 1844 | QVQLVQSGSELK | 1845 | GYTF | 1846 | INTN | 1847 | APRY | 1848 |
| | SLSPGERATLS | | SY | | | | NWL | | KPGASVKISCKA | | TSYA | | TGNP | | SSSW | |
| | CRASQSVSSY | | | | | | MYT | | SGYTFTSYAMN | | | | | | YLDY | |
| | LAWYQQKPG | | | | | | | | WVRQAPGQGLE | | | | | | | |
| | QAPRLLIYDAS | | | | | | | | SMGWINTNTGNP | | | | | | | |
| | NRATGIPARFS | | | | | | | | TYAQGFTGRFVF | | | | | | | |
| | GSGSGTDFTLT | | | | | | | | SMDTSVSTAYLQ | | | | | | | |
| | ISSLEPEDFAV | | | | | | | | ISSLKAEDTAIYY | | | | | | | |
| | YYCQQRSNW | | | | | | | | CAPRYSSSWYLD | | | | | | | |
| | LMYTFGQGTK | | | | | | | | YWGQGTLVTVSS | | | | | | | |
| | LEIK | | | | | | | | | | | | | | | |
| VEGFR2 | DIQMTQSPSSL | 1849 | QDIA | 1850 | ATS | 1851 | LQY | 1852 | EVQLVESGGGLV | 1853 | GFTF | 1854 | ITSG | 1855 | VRIG | 1856 |
| | SASVGDRVTIT | | GS | | | | GSFP | | QPGGSLRLSCAA | | SSYG | | GSYT | | EDAL | |
| | CRASQDIAGSL | | | | | | PT | | SGFTFSSYGMSW | | | | | | DY | |
| | NWLQQKPGK | | | | | | | | VRQAPGKGLEW | | | | | | | |
| | AIKRLIYATSS | | | | | | | | VATITSGGSYTY | | | | | | | |
| | LDSGVPKRFS | | | | | | | | YVDSVKGRFTIS | | | | | | | |
| | GSRSGSDYTL | | | | | | | | RDNAKNTLYLQ | | | | | | | |
| | TISSLQPEDFA | | | | | | | | MNSLRAEDTAV | | | | | | | |
| | TYYCLQYGSF | | | | | | | | YYCVRIGEDALD | | | | | | | |
| | PPTFGQGTKV | | | | | | | | YWGQGTLVTVSS | | | | | | | |
| | EIK | | | | | | | | | | | | | | | |
| VEGFR2 | DIQMTQSPSSV | 1857 | QGID | 1858 | DAS | 1859 | QQA | 1860 | EVQLVQSGGGLV | 1861 | GFTF | 1862 | ISSS | 1863 | ARVT | 1864 |
| | SASIGDRVTIT | | NW | | | | KAFP | | KPGGSLRLSCAA | | SSYS | | SSYI | | DAFD | |
| | CRASQGIDNW | | | | | | PT | | SGFTFSSYSMNW | | | | | | I | |
| | LGWYQQKPG | | | | | | | | VRQAPGKGLEW | | | | | | | |
| | KAPKLLIYDAS | | | | | | | | VSSISSSSSYIYY | | | | | | | |
| | NLDTGVPSRFS | | | | | | | | ADSVKGRFTISR | | | | | | | |
| | GSGSGTYFTLT | | | | | | | | DNAKNSLYLQM | | | | | | | |
| | ISSLQAEDFAV | | | | | | | | NSLRAEDTAVYY | | | | | | | |
| | YFCQQAKAFP | | | | | | | | CARVTDAFDIWG | | | | | | | |
| | PTFGGGTKVDI | | | | | | | | QGTMVTVSS | | | | | | | |
| | K | | | | | | | | | | | | | | | |
| VSIR | DIQMTQSPSSL | 1865 | QSID | 1866 | SAS | 1867 | QQS | 1868 | QVQLVQSGAEV | 1869 | GGTF | 1870 | IIPI | 1871 | ARSS | 1872 |
| | SASVGDRVTIT | | TR | | | | AYN | | KKPGSSVKVSCK | | SSYA | | FGTA | | YGWS | |
| | CRASQSIDTRL | | | | | | PIT | | ASGGTFSSYAIS | | | | | | YEFD | |
| | NWYQQKPGK | | | | | | | | WVRQAPGQGLE | | | | | | Y | |
| | APKLLIYSASS | | | | | | | | WMGGIIPIFGTAN | | | | | | | |
| | LQSGVPSRFSG | | | | | | | | YAQKFQGRVTIT | | | | | | | |
| | SGSGTDFTLTI | | | | | | | | ADESTSTAYMEL | | | | | | | |
| | SSLQPEDFATY | | | | | | | | SSLRSEDTAVYY | | | | | | | |
| | YCQQSAYNPI | | | | | | | | CARSSYGWSYEF | | | | | | | |
| | TFGQGTKVEI | | | | | | | | DYWGQGTLVTV | | | | | | | |
| | K | | | | | | | | SS | | | | | | | |
| CD171 | DIVMSQSPSSL | 1873 | QSLL | 1874 | WAS | 1875 | QQY | 1876 | QVQLQQPGDELV | 1877 | GYTF | 1878 | INPS | 1879 | ALYD | 1880 |
| (L1CAM) | AVSVGEKVTM | | YSSN | | | | HSYP | | KPGASVKLSCKA | | TSY | | NGRT | | GYYA | |
| | SCKSSQSLLYS | | QKNY | | | | FT | | SGYTFTSYWMQ | | W | | | | MDY | |
| | SNQKNYLAW | | | | | | | | WVKQRPGQGLE | | | | | | | |
| | YQQKPGQSPK | | | | | | | | WIGEINPSNGRTN | | | | | | | |
| | LLIYWASTRES | | | | | | | | YNEMFKSKATLT | | | | | | | |
| | GVPDRFTGSG | | | | | | | | VDKSSSTAYMQL | | | | | | | |
| | SGTDFTLTISS | | | | | | | | SSLTSEDSAVYY | | | | | | | |
| | VKAEDLALYY | | | | | | | | CALYDGYYAMD | | | | | | | |
| | CQQYHSYPFT | | | | | | | | YWGQGTSVTVSS | | | | | | | |
| | FGSGTKLEIK | | | | | | | | | | | | | | | |
| CD171 | DIQMTQSSSSF | 1881 | EDIN | 1882 | GAT | 1883 | QQY | 1884 | QVQLQQPGAELV | 1885 | GYTF | 1886 | INPS | 1887 | ARDY | 1888 |
| (L1CAM) | SVSLGDRVTIT | | NR | | | | WSTP | | KPGASVKLSCKA | | TGY | | NGRT | | YGTS | |
| | CKANEDINNR | | | | | | FT | | SGYTFTGYWMH | | W | | | | YNFD | |
| | LAWYQQTPG | | | | | | | | WVKQRPGHGLE | | | | | | Y | |
| | NSPRLLISGAT | | | | | | | | WIGEINPSNGRTN | | | | | | | |
| | NLVTGVPSRFS | | | | | | | | YNERFKSKATLT | | | | | | | |
| | GSGSGKDYTL | | | | | | | | VDKSSTTAFMQL | | | | | | | |
| | TITSLQAEDFA | | | | | | | | SGLTSEDSAVYF | | | | | | | |
| | TYYCQQYWST | | | | | | | | CARDYYGTSYNF | | | | | | | |
| | PFTFGSGTELE | | | | | | | | DYWGQGTTLTV | | | | | | | |
| | IK | | | | | | | | SS | | | | | | | |
| CD19 | EIVLTQSPAIM | 1889 | SGVN | 1890 | DTS | 1891 | HQR | 1892 | QVQLVQPGAEV | 1893 | GYTF | 1894 | IDPS | 1895 | ARGS | 1896 |
| | SASPGERVTM | | Y | | | | GSYT | | VKPGASVKLSCK | | TSN | | DSYT | | NPYY | |
| | TCSASSGVNY | | | | | | | | TSGYTFTSNWMH | | W | | | | YAMD | |
| | MHWYQQKPG | | | | | | | | WVKQAPGQGLE | | | | | | Y | |
| | TSPRRWIYDTS | | | | | | | | WIGEIDPSDSYTN | | | | | | | |
| | KLASGVPARF | | | | | | | | YNQNFQGKAKL | | | | | | | |
| | SGSGSGTSYSL | | | | | | | | TVDKSTSTAYME | | | | | | | |
| | TISSMEPEDAA | | | | | | | | VSSLRSDDTAVY | | | | | | | |
| | TYYCHQRGSY | | | | | | | | YCARGSNPYYYA | | | | | | | |
| | TFGGGTKLEIK | | | | | | | | MDYWGQGTSVT | | | | | | | |
| | | | | | | | | | VSS | | | | | | | |
| CD28 | DIQMTQSPSSL | 1897 | QNIY | 1898 | KAS | 1899 | QQG | 1900 | QVQLVQSGAEV | 1901 | GYTF | 1902 | IYPG | 1903 | TRSH | 1904 |
| | SASVGDRVTIT | | VW | | | | QTYP | | KKPGASVKVSCK | | TSYY | | NVNT | | YGLD | |
| | CHASQNIYVW | | | | | | YT | | ASGYTFTSYYIH | | | | | | WNFD | |
| | LNWYQQKPG | | | | | | | | WVRQAPGQGLE | | | | | | V | |
| | KAPKLLIYKAS | | | | | | | | WIGCIYPGNVNT | | | | | | | |
| | NLHTGVPSRFS | | | | | | | | NYNEKFKDRATL | | | | | | | |
| | GSGSGTDFTLT | | | | | | | | TVDTSISTAYME | | | | | | | |
| | ISSLQPEDFAT | | | | | | | | LSRLRSDDTAVY | | | | | | | |
| | YYCQQGQTYP | | | | | | | | FCTRSHYGLDWN | | | | | | | |
| | YTFGGGTKVE | | | | | | | | FDVWGQGTTVT | | | | | | | |
| | IK | | | | | | | | VSS | | | | | | | |
| CD4 | DIQMTQSPSSL | 1905 | QDIN | 1906 | HTS | 1907 | IQYN | 1908 | EVILVESGGAIVE | 1909 | GFTF | 1910 | ISDH | 1911 | ARKY | 1912 |
| | SASLGGKVTIA | | NY | | | | DLFL | | PGGSLKLSCSAS | | SNY | | STNT | | GGDY | |
| | CKASQDINNYI | | | | | | TT | | GFTFSNYAMSW | | A | | | | DPED | |
| | AWYQHKPGK | | | | | | | | VRQTPEKRLEWV | | | | | | Y | |
| | GPRLLIYHTST | | | | | | | | AAISDHSTNTYY | | | | | | | |
| | LQPGIPSRFSG | | | | | | | | PDSVKGRFTISRD | | | | | | | |
| | SGSGRDYSFSI | | | | | | | | NAKNTLYLQMN | | | | | | | |
| | SNLEPEDIATY | | | | | | | | SLRSEDTAIYYCA | | | | | | | |
| | YCIQYNDLFLT | | | | | | | | RKYGGDYDPED | | | | | | | |
| | TFGGGTKLEIK | | | | | | | | YWGQGTTLTVSS | | | | | | | |
| CD47 | DVLMTQTPLS | 1913 | QSIV | 1914 | KVS | 1915 | FQGS | 1916 | QVQLQQPGAELV | 1917 | GYTF | 1918 | IYPG | 1919 | ARGG | 1920 |
| | LPVSLGDQASI | | YSNG | | | | HVP | | KPGASVMMSCK | | TNY | | NDDT | | YRAM | |
| | SCRSSQSIVYS | | NTY | | | | YT | | ASGYTFTNYNM | | DYN | | | | | |
| | NGNTYLGWY | | | | | | | | HWVKQTPGQGL | | | | | | | |
| | LQKPGQSPKL | | | | | | | | EWIGTIYPGNDD | | | | | | | |
| | LIYKVSNRFSG | | | | | | | | TSYNQKFKDKAT | | | | | | | |
| | VPDRFSGSGS | | | | | | | | LTADKSSSAAYM | | | | | | | |
| | GTDFTLKISRV | | | | | | | | QLSSLTSEDSAV | | | | | | | |
| | EAEDLGVYHC | | | | | | | | YYCARGGYRAM | | | | | | | |
| | FQGSHVPYTF | | | | | | | | DYWGQGTSVTV | | | | | | | |
| | GGGTKVEIK | | | | | | | | SS | | | | | | | |
| CD8 | DVQINQSPSFL | 1921 | RSIS | 1922 | SGS | 1923 | QQH | 1924 | QVQLQQSGAELV | 1925 | GFNI | 1926 | IDPA | 1927 | GRGY | 1928 |
| | AASPGETITNC | | QY | | | | NENP | | KPGASVKLSCTA | | KDT | | NDNT | | GYYV | |
| | RTSRSISQYLA | | | | | | LT | | SGFNIKDTYIHFV | | Y | | | | FDH | |
| | WYQEKPGKT | | | | | | | | RQRPEQGLEWIG | | | | | | | |
| | NKLLIYSGSTL | | | | | | | | RIDPANDNTLYA | | | | | | | |
| | QSGIPSRFSGS | | | | | | | | SKFQGKATITAD | | | | | | | |
| | GSGTDFTLTIS | | | | | | | | TSSNTAYMHLCS | | | | | | | |
| | GLEPEDFAMY | | | | | | | | LTSGDTAVYYCG | | | | | | | |
| | YCQQHNENPL | | | | | | | | RGYGYYVFDHW | | | | | | | |
| | TFGAGTKLEL | | | | | | | | GQGTTLTVSS | | | | | | | |
| | K | | | | | | | | | | | | | | | |
| KIR2DL | EIVLTQSPVTL | 1929 | QSVS | 1930 | DAS | 1931 | QQRS | 1932 | QVQLVQSGAEV | 1933 | GGTF | 1934 | FIPI | 1935 | ARIP | 1936 |
| 2 | SLSPGERATLS | | SY | | | | NWM | | KKPGSSVKVSCK | | SFYA | | FGAA | | SGSY | |
| | CRASQSVSSY | | | | | | YT | | ASGGTFSFYAIS | | | | | | YYDY | |
| | LAWYQQKPG | | | | | | | | WVRQAPGQGLE | | | | | | DMDV | |
| | QAPRLLIYDAS | | | | | | | | WMGGFIPIFGAA | | | | | | | |
| | NRATGIPARFS | | | | | | | | NYAQKFQGRVTI | | | | | | | |
| | GSGSGTDFTLT | | | | | | | | TADESTSTAYME | | | | | | | |
| | ISSLEPEDFAV | | | | | | | | LSSLRSDDTAVY | | | | | | | |
| | YYCQQRSNW | | | | | | | | YCARIPSGSYYY | | | | | | | |
| | MYTFGQGTKL | | | | | | | | DYDMDVWGQGT | | | | | | | |
| | EIK | | | | | | | | TVTVSS | | | | | | | |
| pMHC | QSELTQPRSVS | 1937 | SRDV | 1938 | DVI | 1939 | WSF | 1940 | EVQLLESGGGLV | 1941 | GFTF | 1942 | IVSS | 1943 | AGEL | 1944 |
| [NY- | GSPGQSVTISC | | GGYN | | | | AGS | | QPGGSLRLSCAA | | STYQ | | GGST | | LPYY | |
| ESO1] | TGTSRDVGGY | | Y | | | | YYV | | SGFTFSTYQMSW | | | | | | GMDV | |
| | NYVSWYQQH | | | | | | | | VRQAPGKGLEW | | | | | | | |
| | PGKAPKLIIHD | | | | | | | | VSGIVSSGGSTAY | | | | | | | |
| | VIERSSGVPDR | | | | | | | | ADSVKGRFTISR | | | | | | | |
| | FSGSKSGNTAS | | | | | | | | DNSKNTLYLQM | | | | | | | |
| | LTISGLQAEDE | | | | | | | | NSLRAEDTAVYY | | | | | | | |
| | ADYYCWSFA | | | | | | | | CAGELLPYYGM | | | | | | | |
| | GSYYVFGTGT | | | | | | | | DVWGQGTTVTV | | | | | | | |
| | DVTVL | | | | | | | | SS | | | | | | | |
| pMHC | QSVLTQPPSVS | 1945 | SSNI | 1946 | GNS | 1947 | QSY | 1948 | EVQLQQSGAEVK | 1949 | GGTF | 1950 | IIPI | 1951 | ARDV | 1952 |
| [MART1] | GAPGQRVTISC | | GAGY | | | | DNSL | | KPGSSVKVSCKA | | SSYA | | LGIA | | GSGS | |
| | TGSSSNIGAGY | | D | | | | SSW | | SGGTFSSYAISW | | | | | | YSLD | |
| | DVHWYQQLP | | | | | | V | | VRQAPGQGLEW | | | | | | Y | |
| | GTAPKLLIYG | | | | | | | | MGRIIPILGIANY | | | | | | | |
| | NSNRPSGVPD | | | | | | | | AQKFQGRVTITA | | | | | | | |
| | RFSGSKSGTSA | | | | | | | | DKSTSAYMELSS | | | | | | | |
| | SLAITGLQAED | | | | | | | | LRSEDTAVYYCA | | | | | | | |
| | EADYYCQSYD | | | | | | | | RDVGSGSYSLDY | | | | | | | |
| | NSLSSWVFGG | | | | | | | | WGQGTLVTVSS | | | | | | | |
| | GTKLTVL | | | | | | | | | | | | | | | |
| pMHC | SYVLTQPPSVS | 1953 | NIGS | 1954 | DDS | 1955 | QVW | 1956 | EVQLVESGGGLV | 1957 | GFTF | 1958 | ISWN | 1959 | ARGR | 1960 |
| [MAGEA1] | VAPGQTARIT | | RS | | | | DSRT | | QPGRSLRLSCAA | | DDY | | SGSI | | GFHY | |
| | CGGNNIGSRS | | | | | | DHW | | SGFTFDDYAMH | | A | | | | YYYG | |
| | VHWYQQKPG | | | | | | V | | WVRQAPGKGLE | | | | | | MDI | |
| | QAPVLVVYDD | | | | | | | | WVSGISWNSGSI | | | | | | | |
| | SDRPSGIPERF | | | | | | | | GYADSVKGRFTI | | | | | | | |
| | SGSNSGNMAT | | | | | | | | SRDNAKNSLYLQ | | | | | | | |
| | LTISRVEAGDE | | | | | | | | MNSLRAEDTAV | | | | | | | |
| | ADYYCQVWD | | | | | | | | YYCARGRGFHY | | | | | | | |
| | SRTDHWVFGG | | | | | | | | YYYGMDIWGQG | | | | | | | |
| | GTDLTVL | | | | | | | | TTVTVSS | | | | | | | |
| pMHC | EVILTQSPLSL | 1961 | QSLL | 1962 | LGS | 1963 | MQA | 1964 | EVQLVESGGGVV | 1965 | GFTF | 1966 | ISYD | 1967 | ARGG | 1968 |
| [Tyrosi- | PVTPGEPASIS | | HSIG | | | | LQTP | | QPGRSLRLSCAA | | RSY | | G | | GYYE | |
| nase] | CRSSQSLLHSI | | YN | | | | LT | | SGFTFRSYGMHW | | G | | SNK | | TSGP | |
| | GYNYLDWYL | | | | | | | | VRQAPGKGLEW | | | | | | DY | |
| | QKPGQSPQLLI | | | | | | | | VAVISYDGSNKY | | | | | | | |
| | YLGSNRASGV | | | | | | | | YTDSVNGRFTISR | | | | | | | |
| | PDRFSGSGSGT | | | | | | | | DNSKNTLYQMN | | | | | | | |
| | DFTLKISRVEA | | | | | | | | SLRAEDTAVYYC | | | | | | | |
| | EDVGVYYCM | | | | | | | | ARGGGYYETSGP | | | | | | | |
| | QALQTPLTFG | | | | | | | | DYWGQGTLVTV | | | | | | | |
| | GGTKVEIK | | | | | | | | SS | | | | | | | |
| pMHC | DVVMTQSPLS | 1969 | QSLL | 1970 | LGS | 1971 | MQT | 1972 | EVQLVETGGGVV | 1973 | GFTF | 1974 | ISYD | 1975 | AKDR | 1976 |
| [Tyrosi- | PVTPGEPASIS | | HSNG | | | | LQTP | | QPGRSLRLSCAA | | SSYG | | GSNK | | YGWG | |
| nase] | CRSSQSLLHSN | | HNY | | | | LT | | SGFTFSSYGMHW | | | | | | SSFG | |
| | GHNYLDWYL | | | | | | | | VRQAPGKGLEW | | | | | | HDY | |
| | QKPGQSQLLIY | | | | | | | | VAVISYDGSNKY | | | | | | | |
| | LGSNRSGVPD | | | | | | | | YADSVKGRFTIS | | | | | | | |
| | RFSGSGSGTDF | | | | | | | | DNSKNTLYLQM | | | | | | | |
| | TLKISRVEAED | | | | | | | | NSLRAEDTAVYY | | | | | | | |
| | VGVYYCMQT | | | | | | | | CAKDRYGWGSS | | | | | | | |
| | LQTPLTFGPGT | | | | | | | | FGHDYWGQGTL | | | | | | | |
| | KVDIK | | | | | | | | TVSS | | | | | | | |
| pMHC | QSVLTQPPSVS | 1977 | SSNI | 1978 | DNN | 1979 | GTW | 1980 | EVQLVQSGAEVK | 1981 | GYTF | 1982 | INPS | 1983 | ARDG | 1984 |
| [gp100] | AAPGQTVTISC | | GRNY | | | | DSTL | | KPGASVKVSCKA | | TSYY | | GGST | | TYGS | |
| | SGSSSNIGRNY | | | | | | DLY | | SGYTFTSYYIHW | | | | | | GSYP | |
| | VSWFQQVPGR | | | | | | V | | VRQAPGQGLEW | | | | | | YYYY | |
| | APKLLIYDNN | | | | | | | | MGAINPSGGSTP | | | | | | YGMD | |
| | QRPSGIPGRFS | | | | | | | | YAQKFQGRVTM | | | | | | V | |
| | ASKSDTSATL | | | | | | | | TRDTSTSTVYME | | | | | | | |
| | DITGLQSGDE | | | | | | | | LSSLRSEDTAVY | | | | | | | |
| | AVYYCGTWD | | | | | | | | YCARDGTYGSGS | | | | | | | |
| | STLDLYVFGG | | | | | | | | YPYYYYYGMDV | | | | | | | |
| | GTHVPVL | | | | | | | | WGQGTTVTVSS | | | | | | | |
| pMHC | ETTLTQSPGTL | 1985 | ASQS | 1986 | IYGA | 1987 | YCQ | 1988 | EVQLVQSGAEVK | 1989 | GGTF | 1990 | IIPI | 1991 | ARGP | 1992 |
| [MUC1] | SLSLSPGERAT | | VSS | | | | QYG | | KPGSSVKVSCKA | | SSYA | | FGTA | | EYCI | |
| | LSCRASQSVSS | | | | | | SSPR | | SGGTFSSYAISW | | | | | | NGVC | |
| | SYLAWYQQKP | | | | | | T | | VRQAPGQGLEW | | | | | | SLDV | |
| | GQAPRLLIYG | | | | | | | | MGGIIPIFGTANY | | | | | | | |
| | ASSRATGIPDR | | | | | | | | AQKFQGRVTITA | | | | | | | |
| | FSGSGSGTDFT | | | | | | | | DESTSTAYMELS | | | | | | | |
| | LTISRLEPEDF | | | | | | | | SLRSEDTAVYYC | | | | | | | |
| | AVYYCQQYGS | | | | | | | | ARGPEYCINGVC | | | | | | | |
| | SPRTFGQQGT | | | | | | | | SLDVWGQGTTV | | | | | | | |
| | KVEIK | | | | | | | | TVSS | | | | | | | |
| pMHC | EIVMTQSPATL | 1993 | QSVS | 1994 | DAS | 1995 | HQY | 1996 | EVQLVQSGAEVK | 1997 | GGTF | 1998 | IIPI | 1999 | AVHY | 2000 |
| [MUC1] | SLSPGERATLS | | SY | | | | GSSP | | KPGSSVKVSCKA | | SSYA | | FGTA | | GDYV | |
| | CRASQSVSSY | | | | | | QT | | SGGTFSSYAISW | | | | | | FSSM | |
| | LAWYQQKPG | | | | | | | | VRQAPGQGLEW | | | | | | DV | |
| | QAPRLLIYDAS | | | | | | | | MGGIIPIFGTANY | | | | | | | |
| | NRATGIPARFS | | | | | | | | AQKFQGRVTITA | | | | | | | |
| | GSGSGTDFTLT | | | | | | | | DESTSTAYMELS | | | | | | | |
| | ISSLEPEDFAV | | | | | | | | SLRSEDTAVYYC | | | | | | | |
| | YYCHQYGSSP | | | | | | | | AVHYGDYVFSS | | | | | | | |
| | QTFGQGTKVE | | | | | | | | MDVWGQGTTVT | | | | | | | |
| | IK | | | | | | | | VSS | | | | | | | |
| pMHC | EIVLTQSPATL | 2001 | QSVG | 2002 | DAS | 2003 | QQRS | 2004 | EVQLVQSGAEVK | 2005 | GGTF | 2006 | IIPI | 2007 | YCAG | 2008 |
| [tax] | SLSPGERATLS | | SY | | | | NWP | | KPGSSVKVSCKA | | SSYT | | FGTA | | DTDS | |
| | CRASQSVGSY | | | | | | PMY | | SGGTFSSYTISWV | | | | | | SGYY | |
| | LAWYQQKPG | | | | | | T | | RQAPGQGLEWM | | | | | | GAVD | |
| | XAPRLLIYDAS | | | | | | | | GGIIPIFGTANYA | | | | | | Y | |
| | HRATGIPARFS | | | | | | | | QKFQGRVTITAD | | | | | | | |
| | GSGSGTDFTLT | | | | | | | | KSTSTSTAYMEL | | | | | | | |
| | ISSLEPEDFAV | | | | | | | | SSLRSEDTAVYY | | | | | | | |
| | YYCQQRSNWP | | | | | | | | CAGDTDSSGYYG | | | | | | | |
| | PMYTFGQGTK | | | | | | | | AVDYWGQGTLV | | | | | | | |
| | LEIK | | | | | | | | TVSS | | | | | | | |
| pMHC | NFMLTQPHSV | 2009 | GGSI | 2010 | EDN | 2011 | QSSD | 2012 | EVQLVQSGGGV | 2013 | GFTF | 2014 | ISYD | 2015 | AKTL | 2016 |
| [gp100] | SESPGKTVTIS | | DNNY | | | | GSK | | VQPGRSLTLSCA | | SSYG | | GSNK | | SAGE | |
| | CTGSGGSIDN | | | | | | VV | | ASGFTFSSYGMH | | | | | | WIGG | |
| | NYVHWYQQR | | | | | | | | WVRQAPGKGLE | | | | | | GAFD | |
| | PGSAPTTVMF | | | | | | | | WVSVISYDGSNK | | | | | | I | |
| | EDNQRPSGVP | | | | | | | | YYADSVKGRFTI | | | | | | | |
| | DRFSGSIDSSS | | | | | | | | SRDNSKNTLYLM | | | | | | | |
| | NSASLVISGLK | | | | | | | | NSLRTEDTAVYY | | | | | | | |
| | TEDEGDYYCQ | | | | | | | | CAKTLSAGEWIG | | | | | | | |
| | SSDGSKVVFG | | | | | | | | GGAFDIWGHGT | | | | | | | |
| | GGTKLTVL | | | | | | | | MVTVSS | | | | | | | |
| pMHC | DIVMTQSPDSL | 2017 | QSLL | 2018 | WAS | 2019 | QQY | 2020 | QVQLQESGPGLV | 2021 | GGSI | 2022 | ISDS | 2023 | ARVR | 2024 |
| [NY- | AVSLGERVTIN | | YTSN | | | | YKSP | | KPSQTLALTCSVI | | SSGD | | GST | | IQGA | |
| ESO1] | CKSSQSLLYTS | | NRNY | | | | L | | GGSISSGDYYWS | | YY | | | | SWGF | |
| | NNRNYLAWY | | | | | | | | WIRQPPGKGLEW | | | | | | FDL | |
| | QLKPGQPPKL | | | | | | | | VGYISDSGSTYN | | | | | | | |
| | LIYWASTRES | | | | | | | | EPSLNSRVTISVD | | | | | | | |
| | GVPDRFSGSG | | | | | | | | TSKNQFSLKLFS | | | | | | | |
| | SGTDFTLTISG | | | | | | | | MTAADTAVYYC | | | | | | | |
| | LQAEDVAVYY | | | | | | | | ARVRIQGASWGF | | | | | | | |
| | CQQYYKSPLF | | | | | | | | FDLWGRGTLVSV | | | | | | | |
| | GQGTKLEIK | | | | | | | | SS | | | | | | | |
| pMHC | EIVMTQSPATL | 2025 | QSFS | 2026 | AAS | 2027 | QQY | 2028 | QVQLVQSGVEV | 2029 | GYTF | 2030 | ISVY | 2031 | AREG | 2032 |
| [NY- | SVSPGERATLS | | DD | | | | NNW | | KKPGASVKVSCK | | ASY | | NGKT | | GFYG | |
| ESO1] | CRASQSFSDD | | | | | | PQT | | ASGYTFASYGIS | | G | | | | SGSH | |
| | LAWYQQKPG | | | | | | | | WVRQAPGQGLE | | | | | | YRYF | |
| | QAPRLLIYAAS | | | | | | | | WMGWISVYNGK | | | | | | AMDV | |
| | TRATGIPARFS | | | | | | | | TNPAERHLGRVT | | | | | | | |
| | GRGSGTEFTLT | | | | | | | | MTTDTSTNTAY | | | | | | | |
| | ISSLQSEDSAV | | | | | | | | MELRNLKSDDTA | | | | | | | |
| | YYCQQYNNW | | | | | | | | VYYCAREGGFY | | | | | | | |
| | PQTFGQGTKV | | | | | | | | GSGSHYRYFAM | | | | | | | |
| | EIK | | | | | | | | DVWGQGTTVIVS | | | | | | | |
| | | | | | | | | | S | | | | | | | |
| pMHC | DIVMTQTPLSL | 2033 | QSLV | 2034 | KVS | 2035 | MQG | 2036 | QVQLVQSGGGV | 2037 | GFSF | 2038 | MNWS | 2039 | ARGE | 2040 |
| [NY- | PVTLGQPASLS | | FTDG | | | | THW | | VRPGGSLRLSCA | | IDYG | | GDKK | | YSNR | |
| ESO1] | CRSSQSLVFTD | | NTY | | | | PPI | | ASGFSFIDYGMS | | | | | | | |
| | GNTYLNWFQ | | | | | | | | WVRQVPGKGLE | | | | | | | |
| | QRPGQSPRRLI | | | | | | | | WVAGMNWSGD | | | | | | | |
| | YKVSSRDPGV | | | | | | | | KKGHAESVKGRF | | | | | | | |
| | PDRFSGTGSGT | | | | | | | | IISRDNAKNTLYL | | | | | | | |
| | DFTLEISRVEA | | | | | | | | EMSSLRVEDTAL | | | | | | | |
| | EDIGVYYCMQ | | | | | | | | YFCARGEYSNRF | | | | | | | |
| | GTHWPPIFGQ | | | | | | | | DPRGRGTLVTVS | | | | | | | |
| | GTKVEIK | | | | | | | | S | | | | | | | |
| pMHC | EIVLTQSPGTL | 2041 | QSVS | 2042 | GAS | 2043 | QHY | 2044 | EVQLQESGPGLV | 2045 | GGSI | 2046 | IYPR | 2047 | AREY | 2048 |
| [NY- | SLSPGERATLS | | SSY | | | | DNSL | | KPSETLSLTCTVS | | SSDY | | GTS | | YYVT | |
| ESO1] | CRASQSVSSSY | | | | | | ITFG | | GGSISSDYWTWI | | | | | | NGYF | |
| | LGWYQQKPG | | | | | | HGT | | RQPAGKGLEWIG | | | | | | SPGF | |
| | QAPRLLIYGAS | | | | | | R | | RIYPRGTSNYNPS | | | | | | DY | |
| | IRATGIPDRFS | | | | | | | | LKSRVTMSVDTS | | | | | | | |
| | GSGSGTDFTLT | | | | | | | | KNQISLRLSSVTA | | | | | | | |
| | ISRLEPDDFAV | | | | | | | | ADTAVYYCARE | | | | | | | |
| | YYCQHYDNSL | | | | | | | | YYYVTNGYFSPG | | | | | | | |
| | ITFGHGTRLDI | | | | | | | | FDYWGQGTLVT | | | | | | | |
| | K | | | | | | | | VSS | | | | | | | |
| pMHC | DIVMTQSPLSL | 2049 | QSLH | 2050 | LVS | 2051 | MQA | 2052 | EVQLVESGGGVV | 2053 | GFIF | 2054 | ISSD | 2055 | GTGH | 2056 |
| [NY- | PVTPGEPASIS | | SNGY | | | | VQTP | | QPGKSLRLSCAA | | SSFA | | GSNE | | STEY | |
| ESO1] | CRSSQSLHSN | | NY | | | | FT | | SGFIFSSFAVHWV | | | | | | YDGL | |
| | GYNYLDWYL | | | | | | | | RQAPGKGLEWV | | | | | | LGV | |
| | QKPGQSPQLLI | | | | | | | | ATISSDGSNEDY | | | | | | | |
| | YLVSNRASGV | | | | | | | | VDSVKGRFIISRD | | | | | | | |
| | PDRFSGTGSGT | | | | | | | | NSKNTLYLQMNS | | | | | | | |
| | DFTLKISRVEA | | | | | | | | LRRDDTAVYYC | | | | | | | |
| | EDVGVYYCM | | | | | | | | GTGHSTEYYDGL | | | | | | | |
| | QAVQTPFTFG | | | | | | | | LGVWGHGTTVS | | | | | | | |
| | PGTKVDIK | | | | | | | | VSS | | | | | | | |
| pMHC | QSVVTQPPSVS | 2057 | SSNI | 2058 | EDD | 2059 | ATW | 2060 | QLQLQESGPGLV | 2061 | GGSI | 2062 | IYYS | 2063 | ARHV | 2064 |
| [MARTI] | AAPGRKVTISC | | GSNY | | | | DRT | | KPSETLSLTCTVS | | SSSS | | GT | | GHEL | |
| | SGSSSNIGSNY | | | | | | VNV | | GGSISSSSYYWG | | YY | | | | DY | |
| | VSWYQQVPGT | | | | | | VR | | WIRQPPGKGLEW | | | | | | | |
| | APKLLIYEDD | | | | | | | | GSIYYSGTYYNPS | | | | | | | |
| | KRPSGIPDRFS | | | | | | | | LKSRVTISVDTSK | | | | | | | |
| | GSKGTSATLGI | | | | | | | | NQFSLKLSSTAA | | | | | | | |
| | TGLQTGDEAD | | | | | | | | DTAVYYCARHV | | | | | | | |
| | YFCATWDRTV | | | | | | | | GHELDYWGQGT | | | | | | | |
| | NVVRFGGGTR | | | | | | | | LVTVSS | | | | | | | |
| | LTV | | | | | | | | | | | | | | | |
| pMHC | DVVMTQSPLS | 2065 | QSLL | 2066 | LGS | 2067 | MQA | 2068 | QLQLQESGPGLV | 2069 | GGSI | 2070 | IYHS | 2071 | VGSP | 2072 |
| [Tyrosi- | LPVTPGEPASI | | HSIG | | | | LQTP | | KPSGTLSLTCAVS | | SSSN | | GST | | YGDY | |
| nase] | SCRSSQSLLHS | | YNY | | | | PT | | GGSISSSNWWSW | | W | | | | VLDY | |
| | IGYNYLHWFL | | | | | | | | VRQPPGKGLEWI | | | | | | | |
| | QKGQSPQLLIY | | | | | | | | GEIYHSGSTNYN | | | | | | | |
| | LGSNRASGVP | | | | | | | | PSLKSRVTISDKS | | | | | | | |
| | DRFSGSGSGT | | | | | | | | KNQFSLKLSSVT | | | | | | | |
| | DFTLKISRVEA | | | | | | | | AADTAVYYCVG | | | | | | | |
| | EDVGVYYCM | | | | | | | | SPYGDYVLDYW | | | | | | | |
| | QALQTPPTFG | | | | | | | | GQGTLVTVSS | | | | | | | |
| | QGTRLEIK | | | | | | | | | | | | | | | |
| pMHC | QAVVTQPPSA | 2073 | SSNI | 2074 | SNN | 2075 | AAW | 2076 | QMQLVQSGAEV | 2077 | GYSF | 2078 | VDPG | 2079 | ARVQ | 2080 |
| [WT-1] | SGTPGQRVTIS | | GSNT | | | | DDSL | | KEPGESLRISCKG | | TNF | | YSYS | | YSGY | |
| | CSGSSSNIGSN | | | | | | NGW | | SGYSFTNFWISW | | W | | | | YDWF | |
| | TVNWYQQVP | | | | | | V | | VRQMPGKGLEW | | | | | | DP | |
| | GTAPKLLIYSN | | | | | | | | MGRVDPGYSYST | | | | | | | |
| | NQRPSGVPDR | | | | | | | | YSPSFQGHVTISA | | | | | | | |
| | FSGSKSGTSAS | | | | | | | | DKSTSTAYLQWN | | | | | | | |
| | LAISGLQSEDE | | | | | | | | SLKASDTAMYYC | | | | | | | |
| | ADYYCAAWD | | | | | | | | ARVQYSGYYDW | | | | | | | |
| | DSLNGWVFGG | | | | | | | | FDPWGQGTLVTV | | | | | | | |
| | GTKLTVL | | | | | | | | SS | | | | | | | |
| pMHC | DIVMTQSQKF | 2081 | QNVH | 2082 | LAS | 2083 | LQH | 2084 | QVQLKESGPGLV | 2085 | GFSL | 2086 | IWGD | 2087 | ARDP | 2088 |
| [EBNA-1] | MSTSVGDRVS | | TA | | | | WNN | | APSQSLSITCTVS | | TGY | | GST | | YGYI | |
| | ITCKASQNVH | | | | | | PLT | | GFSLTGYGVNW | | G | | | | FDY | |
| | TAVAWYQQK | | | | | | | | VRQPPGKGLEWL | | | | | | | |
| | AGQSPKALIYL | | | | | | | | GMIWGDGSTDY | | | | | | | |
| | ASNRHTGVPD | | | | | | | | NSALKSRLSISKD | | | | | | | |
| | RFTGSGSGTDF | | | | | | | | NSKSQVFLKMNS | | | | | | | |
| | TLTISNVQSED | | | | | | | | LQTDDTARYYCA | | | | | | | |
| | LADYFCLQHW | | | | | | | | RDPYGYIFDYWG | | | | | | | |
| | NNPLTFGAGT | | | | | | | | QGTTLTVSS | | | | | | | |
| | KLELK | | | | | | | | | | | | | | | |
| pMHC | DIVMTQSQKF | 2089 | QNVF | 2090 | STS | 2091 | QQYI | 2092 | QVQLKQSGPGLV | 2093 | GFSL | 2094 | IWSG | 2095 | ARNW | 2096 |
| [LMP2] | MSTSVGDRVS | | TN | | | | SYPL | | QPSQSLSITCTVS | | TNY | | GST | | VPYY | |
| | VTCRASQNVF | | | | | | T | | GFSLTNYGVHW | | G | | | | FDY | |
| | TNVAWYQQK | | | | | | | | VRQSPGKGLEwl | | | | | | | |
| | PGQAPKALIYS | | | | | | | | GVIWSGGSTDYN | | | | | | | |
| | TSYRYSGVPD | | | | | | | | AAFISRLSISKDN | | | | | | | |
| | RFTGSGSGTDF | | | | | | | | SKQVFFKMNSLQ | | | | | | | |
| | TLTISNVQSED | | | | | | | | ANDTAIYYCARN | | | | | | | |
| | LAEYFCQQYIS | | | | | | | | WVPYYFDYWGQ | | | | | | | |
| | YPLTFGAGTK | | | | | | | | GTTLTVSS | | | | | | | |
| | LELK | | | | | | | | | | | | | | | |
| pMHC | ETTLTQSPGTL | 2097 | QSVS | 2098 | AAS | 2099 | QQY | 2100 | QVQLQESGGGLV | 2101 | GFTF | 2102 | ISSS | 2103 | VRGD | 2104 |
| [gp100] | SLSPGERATLS | | SNY | | | | GSSR | | KPGGSLRLSCAA | | SSYS | | GSTI | | PYFF | |
| | CRASQSVSSN | | | | | | S | | SGFTFSSYSMNW | | | | | | YYYG | |
| | YLAWYQQKP | | | | | | | | VRQAPGKGLEW | | | | | | MDI | |
| | GQAPRLLIYA | | | | | | | | VSYISSSGSTIYY | | | | | | | |
| | ASSRATGIPDR | | | | | | | | ADSVRGRFTISRD | | | | | | | |
| | FSGSGSGTDFT | | | | | | | | NAKNTLYLQMN | | | | | | | |
| | LTISRLEPEDF | | | | | | | | SLRAEDTAVYYC | | | | | | | |
| | AVYYCQQYGS | | | | | | | | VRGDPYFFYYYG | | | | | | | |
| | SRSFGQGTKL | | | | | | | | MDIWGQGTTVT | | | | | | | |
| | EIK | | | | | | | | VSS | | | | | | | |
| pMHC | DIQLTQSPSSL | 2105 | QSIS | 2106 | SAS | 2107 | QQS | 2108 | QVQLQESGPGLV | 2109 | GGSI | 2110 | IDYS | 2111 | ARES | 2112 |
| [gp100] | SASVGDRVIIT | | TH | | | | YSSP | | KPSETLSLTCTVS | | SSN | | GST | | GSPY | |
| | CRATQSISTHL | | | | | | PIT | | GGSISSNMYYWG | | MYY | | | | YFDY | |
| | NWYQQKPGK | | | | | | | | WVRQPPGKGLE | | | | | | | |
| | APKLLIYSASS | | | | | | | | WIGSIDYSGSTYY | | | | | | | |
| | LQSGVPSRFSG | | | | | | | | NPSLRSRVTMSV | | | | | | | |
| | SGSGSTDFTLT | | | | | | | | DTSKKQFSLKMT | | | | | | | |
| | ISSLQPEDFAT | | | | | | | | SVTAADTAVYYC | | | | | | | |
| | YYCQQSYSSP | | | | | | | | ARESGSPYYFDY | | | | | | | |
| | PITFGQGTRLE | | | | | | | | WGQGTLVTVSS | | | | | | | |
| | IK | | | | | | | | | | | | | | | |
| pMHC | ETTLTQSPGTL | 2113 | QSVS | 2114 | GAS | 2115 | QQY | 2116 | QVQLQESGPGLV | 2117 | GGSI | 2118 | WINH | 2119 | ARVV | 2120 |
| [hTERT] | SLSPGERATLS | | SSY | | | | GTSL | | KPSETLSLTCTVS | | SSSS | | SGST | | AAAG | |
| | CRASQSVSSSY | | | | | | TWY | | GGSISSSSYYWA | | YY | | | | HYYY | |
| | LAWYQQKPG | | | | | | | | WIRQPPGKLEWI | | | | | | YYMD | |
| | QAPRLLIYGAS | | | | | | | | GEWINHSGSTNY | | | | | | V | |
| | TRATGVPDRF | | | | | | | | NPSLKSRVTISVD | | | | | | | |
| | SGSGSGTDFTL | | | | | | | | TSKNQFSLNLNS | | | | | | | |
| | ISRLEPEDFAV | | | | | | | | VTAADTAVYYC | | | | | | | |
| | YYCQQYGTSL | | | | | | | | ARVVAAAGHYY | | | | | | | |
| | TWYFGQGTK | | | | | | | | YYYMDVWGKGT | | | | | | | |
| | VEIK | | | | | | | | TVTVSS | | | | | | | |
| pMHC | ETTLTQSPGTL | 2121 | QSVS | 2122 | GAS | 2123 | QQY | 2124 | QVQLQESGPGLV | 2125 | GGSI | 2126 | IYYS | 2127 | ARSR | 2128 |
| [hTERT] | SLSPGERATLS | | SRY | | | | GSSN | | KPSETLSLTCTVS | | SSSY | | GST | | SGSY | |
| | CRASQSVSSR | | | | | | T | | GGSISSSYYWGW | | Y | | | | LNDA | |
| | YLAWYQQKP | | | | | | | | IRQPPGKGLEWIG | | | | | | FDI | |
| | GQAPRLLIYG | | | | | | | | SIYYSGSTYYNPS | | | | | | | |
| | ASSRATGIPDR | | | | | | | | LKSRVTISVDTSK | | | | | | | |
| | FSGSGSGTDFT | | | | | | | | NQFSLKLSSVTA | | | | | | | |
| | LTISRLEPEDF | | | | | | | | ADTAVYYCARSR | | | | | | | |
| | AVYYCQQYGS | | | | | | | | SGSYLNDAFDIW | | | | | | | |
| | SNTFGQGTKL | | | | | | | | GQGTMVTVSS | | | | | | | |
| | EIK | | | | | | | | | | | | | | | |
| pMHC | ETTLTQSPGTL | 2129 | QSVS | 2130 | GAS | 2131 | QQY | 2132 | QVQLQQSGAEV | 2133 | GGTF | 2134 | IIPI | 2135 | ARGF | 2136 |
| [hTERT] | SLSPGERATLS | | SSY | | | | GSSS | | KKPGSSVKVSCK | | SSYA | | LGIA | | RPYY | |
| | CRASQSVSSSY | | | | | | GT | | ASGGTFSSYAIS | | | | | | YYGM | |
| | LAWYQQKPG | | | | | | | | WVRQAPGQGLE | | | | | | DV | |
| | QAPRLLIYGAS | | | | | | | | WMGRIIPILGIAN | | | | | | | |
| | SRATGIPDRFS | | | | | | | | YAQKFQGRVTIT | | | | | | | |
| | GSGSGTDFTLT | | | | | | | | ADKSTSTAYMEL | | | | | | | |
| | ISRLEPEDFAV | | | | | | | | SSLRSEDTAVYY | | | | | | | |
| | YYCQQYGSSS | | | | | | | | CARGFRPYYYYG | | | | | | | |
| | GTFGQGTKVE | | | | | | | | MDVWGQGTTVT | | | | | | | |
| | IK | | | | | | | | VSS | | | | | | | |
| pMHC | QSVVTQPPSVS | 2137 | SSNI | 2138 | GNS | 2139 | QSY | 2140 | QVQLQQSGPGLV | 2141 | GGSI | 2142 | MYYS | 2143 | ARIP | 2144 |
| [gp100] | GAPGQRVTISC | | GAGY | | | | DSSL | | KPSETLSLTCTVS | | RNY | | GGA | | NYYD | |
| | TGSSSNIGAGY | | D | | | | SAL | | GGSIRNYYWSWI | | Y | | | | RSGY | |
| | DVHWYQQLP | | | | | | | | RQPPGKGLEWIG | | | | | | YPGY | |
| | GTAPKLLIYG | | | | | | | | YMYYSGGANYN | | | | | | WYFD | |
| | NSNRPSGVPD | | | | | | | | PSLNSRVTISLDT | | | | | | L | |
| | RFSGSKSGTSA | | | | | | | | SKNQFSLKLTSV | | | | | | | |
| | SLAITGLQAED | | | | | | | | TAADTAVYYCA | | | | | | | |
| | EADYYCQSYD | | | | | | | | RIPNYYDRSGYY | | | | | | | |
| | SSLSALFGGGT | | | | | | | | PGYWYFDLWGR | | | | | | | |
| | KLTVL | | | | | | | | GTLVTVSS | | | | | | | |
| pMHC | DIQLTQSPSSL | 2145 | QSIS | 2146 | SAS | 2147 | QQS | 2148 | QVQLQQSGPGLV | 2149 | GDSI | 2150 | TYYR | 2151 | ARAS | 2152 |
| [gp100] | SASVGDRVTIT | | TY | | | | DIIP | | KPSQTLSLTCAIS | | SSNS | | SKWY | | FGTS | |
| | CRASQSISTYL | | | | | | LT | | GDSISSNSVVWN | | VV | | N | | GKFD | |
| | NWYQHRPGK | | | | | | | | WIRQSPSRGLEW | | | | | | D | |
| | APKLLIYSASS | | | | | | | | LGRTYYRSKWY | | | | | | | |
| | LQSGVPSRFSG | | | | | | | | NDYAVSVKSRITI | | | | | | | |
| | SGSGTDFTLTI | | | | | | | | NPDTSKNQFSLQ | | | | | | | |
| | SSLQPEDFATY | | | | | | | | LNSVTPDDTALY | | | | | | | |
| | YCQQSDIIPLT | | | | | | | | YCARASFGTSGK | | | | | | | |
| | FGGGTKVEIN | | | | | | | | FDDWGQGTLVT | | | | | | | |
| | | | | | | | | | VSS | | | | | | | |
| pMHC | SYVLTQPPSVS | 2153 | TIGR | 2154 | DDT | 2155 | QVW | 2156 | QVQLQQSGPGLV | 2157 | GDS | 2158 | TYYR | 2159 | CVRG | 2160 |
| [hTERT] | EAPGKTARITC | | KS | | | | DSST | | KPSQTLSLTCAIS | | VSSK | | SKWY | | SIFD | |
| | EGITIGRKSVH | | | | | | DPQ | | GDSVSSKNSSWN | | NSS | | Y | | V | |
| | WYQQKPGQA | | | | | | VV | | WIRQSPSRGLEW | | | | | | | |
| | PVLVVYDDTV | | | | | | | | LGRTYYRSKWY | | | | | | | |
| | RPSGVPERFSG | | | | | | | | YDYAVSVKGRIT | | | | | | | |
| | SNSGNTATLII | | | | | | | | FTFPDTSKNQVSL | | | | | | | |
| | SGVEAGDEAD | | | | | | | | HLNAVTPEDTAM | | | | | | | |
| | YCQVWDSSTD | | | | | | | | YYCVRGSIFDVW | | | | | | | |
| | PQVVFGGGTK | | | | | | | | GQGTMVTVSS | | | | | | | |
| | TVL | | | | | | | | | | | | | | | |
| pMHC | NFMLTQPHSV | 2161 | GGSI | 2162 | EDD | 2163 | QSY | 2164 | QVQLQQWGAGL | 2165 | GGSF | 2166 | INHS | 2167 | ARMV | 2168 |
| [hTERT] | SESPGKTVTIS | | ATNY | | | | DSSN | | LKPSETLSLTCAV | | SGY | | GST | | RYYY | |
| | CTGSGGSIATN | | | | | | QV | | YGGSFSGYYWS | | Y | | | | GMDV | |
| | YVQWYQQRP | | | | | | | | WIRQPPGKGLEW | | | | | | | |
| | GSAPATVIYED | | | | | | | | IGEINHSGSTNYN | | | | | | | |
| | DQRPSGVPDR | | | | | | | | PSLKSRVTISVDT | | | | | | | |
| | FSGSIDSSSNS | | | | | | | | SKNQFSLKLSSVT | | | | | | | |
| | ASLTISGLKTE | | | | | | | | AADTAVYYCAR | | | | | | | |
| | DEADYYCQSY | | | | | | | | MVRYYYGMDV | | | | | | | |
| | DSSNQVFGGG | | | | | | | | WGQGTTVTVSS | | | | | | | |
| | TKLTVL | | | | | | | | | | | | | | | |
| pMHC | ETTLTQSPGTL | 2169 | QSVG | 2170 | GAS | 2171 | QQY | 2172 | QVQLQQWGAGL | 2173 | GGSF | 2174 | INHS | 2175 | ARVA | 2176 |
| [hTERT] | SLSPGERATLS | | SN | | | | GDSP | | LKPSETLSLTCAV | | SGY | | GST | | YYDS | |
| | CRASQSVGSN | | | | | | RLYT | | YGGSFSGYYWS | | Y | | | | SGYY | |
| | LAWYQQRPG | | | | | | | | WIRQPPGKGLEW | | | | | | PYDA | |
| | QAPSLLIYGAS | | | | | | | | IGEINHSGSTNYN | | | | | | FDI | |
| | SRATGVPDRF | | | | | | | | PSLKSRVTISVDT | | | | | | | |
| | SGSGSGTDFTL | | | | | | | | SKNQFSLKLSSVT | | | | | | | |
| | TISRLEPEDFA | | | | | | | | AADTAVYYCAR | | | | | | | |
| | VYYCQQYGDS | | | | | | | | VAYYDSSGYYPY | | | | | | | |
| | PRLYTFGQGT | | | | | | | | DAFDIWGQGTM | | | | | | | |
| | KLEIK | | | | | | | | VTVSS | | | | | | | |
| pMHC | DVVMTQSPGT | 2177 | QLSD | 2178 | SAS | 2179 | HQY | 2180 | QVQLVQSGAEV | 2181 | GYTF | 2182 | ISSS | 2183 | ARYD | 2184 |
| [gp100] | LSVSPGDSATL | | SY | | | | GFLP | | KKPGASVKVSCK | | TRY | | NGYT | | ISGL | |
| | SCWASQLSDS | | | | | | WT | | ASGYTFTRYGIS | | G | | | | DGFD | |
| | YVSWYQQKP | | | | | | | | WVRQAPGQGLE | | | | | | I | |
| | GQAPRLLIHSA | | | | | | | | WMGWISSSNGY | | | | | | | |
| | SIRAPGIPDRFS | | | | | | | | TKYAQNLQGRLT | | | | | | | |
| | GSVSGTEFTLT | | | | | | | | LTTDTSTGTAYM | | | | | | | |
| | ISGLEPEDFAV | | | | | | | | ELRSLRSEDTAL | | | | | | | |
| | YSCHQYGFLP | | | | | | | | YYCARYDISGLD | | | | | | | |
| | WTFGQGTKVE | | | | | | | | GFDIWGQGTMV | | | | | | | |
| | IR | | | | | | | | TVSS | | | | | | | |
| pMHC | ETTLTQSPGTL | 2185 | RYIN | 2186 | DAS | 2187 | QQY | 2188 | QVQLVQSGAEV | 2189 | GGTF | 2190 | IIPI | 2191 | CARD | 2192 |
| [gp100] | SLSPGERATLS | | ANF | | | | GSSP | | KKPGSSVKVSCK | | SSYA | | FGTA | | SSGW | |
| | CRASRYINAN | | | | | | RT | | ASGGTFSSYAIS | | | | | | LYDA | |
| | FLAWYQQKPG | | | | | | | | WVRQAPGQGLE | | | | | | FDI | |
| | QAPRLLIYDAS | | | | | | | | WMGGIIPIFGTAT | | | | | | | |
| | TRATGIPDRFS | | | | | | | | NYAQKFQGRVTI | | | | | | | |
| | GSGSGTDFTLT | | | | | | | | TADESTSTAYME | | | | | | | |
| | ISRLEPEDFAV | | | | | | | | LSSLRSEDTAVY | | | | | | | |
| | YYCQQYGSSP | | | | | | | | YCARDSSGWLY | | | | | | | |
| | RTFGQGTKVEI | | | | | | | | DAFDIWGQGTM | | | | | | | |
| | K | | | | | | | | VTVSS | | | | | | | |
| pMHC | DIQMTQSPSIL | 2193 | QRFG | 2194 | GAS | 2195 | QQA | 2196 | QVQLVQSGAEV | 2197 | GGTF | 2198 | INVG | 2199 | ARDG | 2200 |
| [hTERT] | SASVGDRVTIT | | DY | | | | NSFP | | KKPGSSVKVSCK | | SSYA | | NGNA | | ERAW | |
| | CRASQRFGDY | | | | | | ITFG | | ASGGTFSSYAIS | | | | | | DLDY | |
| | LAWYQQKPG | | | | | | KGT | | WVRQAPGQGLE | | | | | | | |
| | QAPKLLIYGAS | | | | | | R | | WMGWINVGNGN | | | | | | | |
| | TLQSGVPSRFS | | | | | | | | AIYSQKFQGRVTI | | | | | | | |
| | GSGSGTEFTLT | | | | | | | | TRDTSATTAYME | | | | | | | |
| | ISGLQPEDFAT | | | | | | | | LSSLRSEDTAVY | | | | | | | |
| | YYCQQANSFPI | | | | | | | | YCARDGERAWD | | | | | | | |
| | TFGKGTRLDIR | | | | | | | | LDYWGQGTLVT | | | | | | | |
| | | | | | | | | | VSS | | | | | | | |
| pMHC | ETTLTQSPGTL | 2201 | QSVS | 2202 | GAS | 2203 | QQY | 2204 | QVQLVQSGGGV | 2205 | GFTF | 2206 | ISYD | 2207 | AREL | 2208 |
| [hTERT] | SLSPGERATLS | | SSY | | | | GSSP | | VQPGRSLRLSCA | | SSYA | | GSNK | | RFLE | |
| | CRASQSVSSSY | | | | | | YT | | ASGFTFSSYAMH | | | | | | WSSD | |
| | LAWYQQKPG | | | | | | | | WVRQAPGKGLE | | | | | | AFDI | |
| | QAPRLLIYGAS | | | | | | | | WVAVISYDGSNK | | | | | | | |
| | SRATGIPDRFS | | | | | | | | YYADSVKGRFTI | | | | | | | |
| | GSGSGTDFTLT | | | | | | | | SRDNSKNTLYLQ | | | | | | | |
| | ISRLEPEDFAV | | | | | | | | MNSLRAEDTAV | | | | | | | |
| | YYCQQYGSSP | | | | | | | | YYCARELRFLEW | | | | | | | |
| | YTFGQGTKLEI | | | | | | | | SSDAFDIWGQGT | | | | | | | |
| | K | | | | | | | | MVTVSS | | | | | | | |
| pMHC | ETTLTQSPGTL | 2209 | QSVS | 2210 | GAS | 2211 | QQH | 2212 | QVQLVQSGGGV | 2213 | GFTF | 2214 | ISYD | 2215 | AKDS | 2216 |
| [gp100] | SLSPGERATLS | | SSY | | | | DSSP | | VQPGRSLRLSCA | | SSYG | | GSDK | | YYDN | |
| | CRASQSVSSSY | | | | | | RT | | ASGFTFSSYGMH | | | | | | SAFQ | |
| | LAWYQQKPG | | | | | | | | WVRQAPGKGLE | | | | | | AD | |
| | QAPRLLIYGAS | | | | | | | | WVAFISYDGSDK | | | | | | | |
| | SRATGIPDRFS | | | | | | | | NFADSVKGRFTIS | | | | | | | |
| | GSGSGTDFTLT | | | | | | | | RDNSKNTLYLQ | | | | | | | |
| | ISRLEPEDFAV | | | | | | | | MNSLRAEDTAV | | | | | | | |
| | YYCQQHDSSP | | | | | | | | YYCAKDSYYDN | | | | | | | |
| | RTFGQGTKVEI | | | | | | | | SAFQADWGQGT | | | | | | | |
| | K | | | | | | | | LVTVSS | | | | | | | |
| pMHC | EIVLTQSPLSL | 2217 | QSLL | 2218 | LGS | 2219 | MQA | 2220 | QVQLVQSGGGV | 2221 | GFTF | 2222 | ISYD | 2223 | ARDF | 2224 |
| [tax] | PVTPGEPASIS | | HSNG | | | | LQTP | | VQPGRSLRLSCA | | SSYG | | GSNK | | DYGD | |
| | CRSSQSLLHSN | | YNY | | | | RT | | ASGFTFSSYGMH | | | | | | SYYY | |
| | GYNYLDWYL | | | | | | | | WVRQAPGKGLE | | | | | | YGMD | |
| | QKPGQSPQLLI | | | | | | | | WVAVISYDGSNK | | | | | | V | |
| | YLGSNRASGV | | | | | | | | YYADSVKGRFTI | | | | | | | |
| | PDRFSGSGSGT | | | | | | | | SRDNSKNTLYLQ | | | | | | | |
| | DFTLKISRVEA | | | | | | | | MNSLRAEDTAV | | | | | | | |
| | EDVGVYYCM | | | | | | | | YYCARDFDYGDS | | | | | | | |
| | QALQTPRTFG | | | | | | | | YYYYGMDVWG | | | | | | | |
| | QGTKVEIK | | | | | | | | QGTTVTVSS | | | | | | | |
| pMHC | DVMTQSPLSL | 2225 | QSLL | 2226 | FGS | 2227 | MQA | 2228 | QVQLVQSGGGV | 2229 | GFTF | 2230 | ISYD | 2231 | ARDY | 2232 |
| [gp100] | PVTPGEPASIS | | HSNG | | | | THW | | VQPGRSLRLSCA | | SSYG | | GSNK | | YGDY | |
| | CRSSQSLLHSN | | YKY | | | | PYT | | ASGFTFSSYGMH | | | | | | ALLD | |
| | GYKYVNWYL | | | | | | | | WVRQAPGKGLE | | | | | | Y | |
| | QKPGQSPQLLI | | | | | | | | WVAVISYDGSNK | | | | | | | |
| | YFGSYRASGV | | | | | | | | YYADSVKGRFTI | | | | | | | |
| | PDRFSGSGSGT | | | | | | | | SRDNSKNTLYLQ | | | | | | | |
| | DFTLKISRVEA | | | | | | | | MNSLRAEDTAV | | | | | | | |
| | EDVGIYYCMQ | | | | | | | | YYCARDYYGDY | | | | | | | |
| | ATHWPYTFGQ | | | | | | | | ALLDYWGQGTL | | | | | | | |
| | GTRLEIK | | | | | | | | VTVSS | | | | | | | |
| pMHC | EIVLTQSPDTL | 2233 | SQSV | 2234 | YDT | 2235 | CQQ | 2236 | QVQLVQSGGGV | 2237 | GFTF | 2238 | ISYD | 2239 | AKTV | 2240 |
| [gp100] | SLSPGEREATL | | SHS | | | | YVSS | | VQPGRSLRLSCA | | STYG | | GSNK | | GVTF | |
| | SCRASQSVSHS | | | | | | PLT | | ASGFTFSTYGLH | | | | | | VSDA | |
| | YLAQYQQKPG | | | | | | | | WVRQAPGKGLE | | | | | | FDI | |
| | QAPRLLIYDTS | | | | | | | | WVAFISYDGSNK | | | | | | | |
| | SRATDIPDRFS | | | | | | | | YYADSVKGRFTI | | | | | | | |
| | GSGSGTDFTLT | | | | | | | | SRDNSKNTLYLQ | | | | | | | |
| | ISRLEPEDSAV | | | | | | | | MNGLRAEDTAV | | | | | | | |
| | YYCQQYVSSP | | | | | | | | YYCAKTVGVTFV | | | | | | | |
| | LTFGQGTKLEI | | | | | | | | SDAFDIWGQGTM | | | | | | | |
| | K | | | | | | | | VTVSS | | | | | | | |
| pMHC | QSELTQPRSVS | 2241 | SRDV | 2242 | DVI | 2243 | WSF | 2244 | QVQLLESGGGLV | 2245 | GFTF | 2246 | IGSS | 2247 | AGEL | 2248 |
| [NY- | GSPGQSVTISC | | GGYN | | | | AGS | | QPGGSLRLSCAA | | SAY | | GGGT | | LPYY | |
| ESO1] | TGTSRDVGGY | | Y | | | | YYV | | SGFTFSAYGMG | | G | | | | GMDV | |
| | NYVSWYQQH | | | | | | | | WVRQAPGKGLE | | | | | | | |
| | PGKAPKLIIHD | | | | | | | | WVSSIGSSGGGT | | | | | | | |
| | VIIRPSGVPDR | | | | | | | | AYADSVKGRFTI | | | | | | | |
| | FSGSKSGNTAS | | | | | | | | SRDNSKNTLYLQ | | | | | | | |
| | LTISGLQAEDE | | | | | | | | MNSLRAEDTAV | | | | | | | |
| | AHYYCWSFA | | | | | | | | YYCAGELLPYYG | | | | | | | |
| | GSYYVFGTGT | | | | | | | | MDVWGQGTTVT | | | | | | | |
| | DVTVL | | | | | | | | VSS | | | | | | | |
| GD2 | ENVLTQSPAI | 2249 | SSVS | 2250 | STS | 2251 | QQY | 2252 | QVQLKESGPVLV | 2253 | GFSL | 2254 | IWAG | 2255 | AKRS | 2256 |
| | MSASPGEKVT | | SSL | | | | SGYP | | APSQTLSITCTVS | | ASY | | GST | | DDYS | |
| | MTCRASSSVS | | | | | | IT | | GFSLASYNIHWV | | N | | | | WFAY | |
| | SSLYHWYQQK | | | | | | | | RQPPGKGLEWLG | | | | | | | |
| | SGASPKVWIY | | | | | | | | VIWAGGSTNYNS | | | | | | | |
| | STSNLASGVP | | | | | | | | ALMSRLSISKDNS | | | | | | | |
| | GRFSGSGSGTS | | | | | | | | KSQVFLQMNSLQ | | | | | | | |
| | YSLTISSVEAE | | | | | | | | TDDTAMYYCAK | | | | | | | |
| | DAATYYCQQ | | | | | | | | RSDDYSWFAYW | | | | | | | |
| | YSGYPITFGAG | | | | | | | | GCQTLVTVSA | | | | | | | |
| | TKVEVK | | | | | | | | | | | | | | | |
| STEAP_ | DIVMSQSPSSL | 2257 | QSLL | 2258 | WAS | 2259 | QQY | 2260 | DVQVQESGPGLV | 2261 | GYSI | 2262 | ISNS | 2263 | ARER | 2264 |
| 1 | AVSVGEKVTM | | YRSN | | | | YNY | | KPSQSLSLTCTVT | | TSDY | | GST | | NYDY | |
| | SCKSSQSLLYR | | QKNY | | | | PRT | | GYSITSDYAWN | | A | | | | DDYY | |
| | SNQKNYLAW | | | | | | | | WIRQFPGNKLEW | | | | | | YAMD | |
| | YQQKPGQSPK | | | | | | | | MGYISNSGSTSY | | | | | | Y | |
| | LLIYWASTRES | | | | | | | | NPSLKSRISITRDT | | | | | | | |
| | GVPDRFTGSG | | | | | | | | SKNQFFLQLISVT | | | | | | | |
| | SGTDFTLTISS | | | | | | | | TEDTATYYCARE | | | | | | | |
| | VKAEDLAVYY | | | | | | | | RNYDYDDYYYA | | | | | | | |
| | CQQYYNYPRT | | | | | | | | MDYWGQGTTLT | | | | | | | |
| | FGGGTKLEIK | | | | | | | | VSA | | | | | | | |
| a4b7 | DIQMTQSPSSV | 2265 | QGIS | 2266 | GAS | 2267 | QQA | 2268 | QVQLVQSGAEV | 2269 | GYT | 2270 | FDPQ | 2271 | ATGS | 2272 |
| | SASVGDRVTIT | | SW | | | | NSFP | | KKPGASVKVSCK | | LSDL | | DGET | | SSSW | |
| | CRASQGISSW | | | | | | WT | | VSGYTLSDLSIH | | S | | | | FDP | |
| | LAWYQQKPG | | | | | | | | WVRQAPGKGLE | | | | | | | |
| | KAPKLLIYGAS | | | | | | | | WMGGFDPQDGE | | | | | | | |
| | NLESGVPSRFS | | | | | | | | TIYAQKFQGRVT | | | | | | | |
| | GSGSGTDFTLT | | | | | | | | MTEDTSTDTAY | | | | | | | |
| | ISSLQPEDFAN | | | | | | | | MELSSLKSEDTA | | | | | | | |
| | YYCQQANSFP | | | | | | | | VYYCATGSSSSW | | | | | | | |
| | WTFGQGTKVE | | | | | | | | FDPWGQGTLVTV | | | | | | | |
| | IK | | | | | | | | SS | | | | | | | |
| GPC3 | DVVMTQSPLS | 2273 | QSLV | 2274 | KVS | 2275 | SQNT | 2276 | QVQLVQSGAEV | 2277 | GYTF | 2278 | LDPK | 2279 | TRFY | 2280 |
| | LPVTPGEPASI | | HSNR | | | | HVPP | | KKPGASVKVSCK | | TDY | | TGDT | | SYTY | |
| | SCRSSQSLVHS | | NTY | | | | T | | ASGYTFTDYEMH | | E | | | | W | |
| | NRNTYLHWY | | | | | | | | WVRQAPGQGLE | | | | | | | |
| | LQKPGQSPQL | | | | | | | | WMGALDPKTGD | | | | | | | |
| | LIYKVSNRFSG | | | | | | | | TAYSQKFKGRVT | | | | | | | |
| | VPDRFSGSGS | | | | | | | | LTADKSTSTAYM | | | | | | | |
| | GTDFTLKISRV | | | | | | | | ELSSLTSEDTAVY | | | | | | | |
| | EAEDVGVYYC | | | | | | | | YCTRFYSYTYWG | | | | | | | |
| | SQNTHVPPTF | | | | | | | | QGTLVTVSS | | | | | | | |
| | GQGTKLEIK | | | | | | | | | | | | | | | |
| CD262 | SELTQDPAVS | 2281 | SLRS | 2282 | GAN | 2283 | NSA | 2284 | EVQLVQSGGGVE | 2285 | GFTF | 2286 | INWQ | 2287 | AKIL | 2288 |
| (DR5) | VALGQTVRIT | | YY | | | | DSSG | | RPGGSLRLSCAA | | DDY | | GGST | | GAGR | |
| | CSGDSLRSYY | | | | | | NHV | | SGFTFDDYAMS | | A | | | | GWYF | |
| | ASWYQQKPG | | | | | | V | | WVRQAPGKGLE | | | | | | DY | |
| | QAPVLVIYGA | | | | | | | | WVSGINWQGGS | | | | | | | |
| | NNRPSGIPDRF | | | | | | | | TGYADSVKGRVT | | | | | | | |
| | SGSSSGNTASL | | | | | | | | ISRDNAKNSLYL | | | | | | | |
| | TITGAQAEDE | | | | | | | | QMNSLRAEDTA | | | | | | | |
| | ADYYCNSADS | | | | | | | | VYYCAKILGAGR | | | | | | | |
| | SGNHVVFGGG | | | | | | | | GWYFDYWGKGT | | | | | | | |
| | TKLTVL | | | | | | | | TVTVSS | | | | | | | |
| CD80 | ESALTQPPSVS | 2289 | TSNI | 2290 | DIN | 2291 | QSY | 2292 | QVQLQESGPGLV | 2293 | GGSI | 2294 | FYSS | 2295 | VRDR | 2296 |
| | GAPGQKVTIS | | GGYD | | | | DSSL | | KPSETLSLTCAVS | | SGG | | SGNT | | LFSV | |
| | CTGSTSNIGGY | | | | | | NAQ | | GGSISGGYGWG | | YG | | | | VGMV | |
| | DLHWYQQLP | | | | | | VFG | | WIRQPPGKGLEW | | | | | | YNNW | |
| | GTAPKLLIYDI | | | | | | G | | IGSFYSSSGNTYY | | | | | | FDVW | |
| | NKRPSGISDRF | | | | | | | | NPSLKSQVTISTD | | | | | | | |
| | SGSKSGTAAS | | | | | | | | TSKNQFSLKLNS | | | | | | | |
| | LAITGLQTEDE | | | | | | | | MTAADTAVYYC | | | | | | | |
| | ADYYCQSYDS | | | | | | | | VRDRLFSVVGM | | | | | | | |
| | SLNAQVFGGG | | | | | | | | VYNNWFDVWGP | | | | | | | |
| | TRLTVL | | | | | | | | GVLVTVSS | | | | | | | |
| CD22 | DVQVTQSPSS | 2297 | QSLA | 2298 | GIS | 2299 | LQGT | 2300 | EVQLVQSGAEVK | 2301 | GYR | 2302 | INPG | 2303 | TREG | 2304 |
| | LSASVGDRVTI | | NSYG | | | | HQP | | KPGASVKVSCKA | | FTNY | | NNYA | | YGNY | |
| | TCRSSQSLANS | | NTF | | | | YT | | SGYRFTNYWIHW | | W | | | | GAWF | |
| | YGNTFLSWYL | | | | | | | | VRQAPGQGLEWI | | | | | | AY | |
| | HKPGKAPQLLI | | | | | | | | GGINPGNNYATY | | | | | | | |
| | YGISNRFSGVP | | | | | | | | RRKFQGRVTMT | | | | | | | |
| | DRFSGSGSGT | | | | | | | | ADTSTSTVYMEL | | | | | | | |
| | DFTLTISSLQP | | | | | | | | SSLRSEDTAVYY | | | | | | | |
| | EDFATYYCLQ | | | | | | | | CTREGYGNYGA | | | | | | | |
| | GTHQPYTFGQ | | | | | | | | WFAYWGQGTLV | | | | | | | |
| | GTKVEIK | | | | | | | | TVSS | | | | | | | |
| CD23 | DIQMTQSPSSL | 2305 | QDIR | 2306 | VAS | 2307 | LQV | 2308 | EVQLVESGGGLA | 2309 | GFRF | 2310 | ISSS | 2311 | ASLT | 2312 |
| | SASVGDRVTIT | | YY | | | | YSTP | | KPGGSLRLSCAA | | TFNN | | GDPT | | TGSD | |
| | CRASQDIRYY | | | | | | RT | | SGFRFTFNNYYM | | YY | | | | SW | |
| | LNWYQQKPG | | | | | | | | DWVRQAPGQGL | | | | | | | |
| | KAPKLLIYVAS | | | | | | | | EWVSRISSSGDPT | | | | | | | |
| | SLQSGVPSRFS | | | | | | | | WYADSVKGRFTI | | | | | | | |
| | GSGSGTEFTLT | | | | | | | | SRENANNTLFLQ | | | | | | | |
| | VSSLQPEDFAT | | | | | | | | MNSLRAEDTAV | | | | | | | |
| | YYCLQVYSTP | | | | | | | | YYCASLTTGSDS | | | | | | | |
| | RTFGQGTKVEI | | | | | | | | WGQGVLVTVSS | | | | | | | |
| | K | | | | | | | | | | | | | | | |
| CD20 | DIQMTQSPSSL | 2313 | SSVS | 2314 | APS | 2315 | QQW | 2316 | EVQLVESGGGLV | 2317 | GYTF | 2318 | IYPG | 2319 | ARVV | 2320 |
| | SASVGDRVTIT | | Y | | | | SFNP | | QPGGSLRLSCAA | | TSYN | | NGDT | | YYSN | |
| | CRASSSVSYM | | | | | | PT | | SGYTFTSYNMH | | | | | | SYWY | |
| | HWYQQKPGK | | | | | | | | WVRQAPGKGLE | | | | | | FDV | |
| | APKPLIYAPSN | | | | | | | | WVGAIYPGNGDT | | | | | | | |
| | LASGVPSRFSG | | | | | | | | SYNQKFKGRFTIS | | | | | | | |
| | SGSGTDFTLTI | | | | | | | | VDKSKNTLYLQ | | | | | | | |
| | SSLQPEDFATY | | | | | | | | MNSLRAEDTAV | | | | | | | |
| | YCQQWSFNPP | | | | | | | | YYCARVVYYSNS | | | | | | | |
| | TFGQGTKVEI | | | | | | | | YWYFDVWGQGT | | | | | | | |
| | K | | | | | | | | LVTVSS | | | | | | | |
| CD37 | EIVLTQSPATL | 2321 | ENVY | 2322 | FAK | 2323 | QHH | 2324 | EVQLVQSGAEVK | 2325 | GYSF | 2326 | IDPY | 2327 | ARSV | 2328 |
| | SLSPGERATLS | | SY | | | | SDNP | | KPGESLKISCKGS | | TGY | | YGGT | | GPFD | |
| | CRASENVYSY | | | | | | WT | | GYSFTGYNMNW | | N | | | | S | |
| | LAWYQQKPG | | | | | | | | VRQMPGKGLEW | | | | | | | |
| | QAPRLLIYFAK | | | | | | | | MGNIDPYYGGTT | | | | | | | |
| | TLAEGIPARFS | | | | | | | | YNRKFKGQVTIS | | | | | | | |
| | GSGSGTDFTLT | | | | | | | | ADKSISTAYLQW | | | | | | | |
| | ISSLEPEDFAV | | | | | | | | SSLKASDTAMYY | | | | | | | |
| | YYCQHHSDNP | | | | | | | | CARSVGPFDSWG | | | | | | | |
| | WTFGQGTKVE | | | | | | | | QGTLVTVSS | | | | | | | |
| | IK | | | | | | | | | | | | | | | |
| CD22 | DIQMTQSPSSL | 2329 | QSIV | 2330 | KVS | 2331 | FQGS | 2332 | EVQLVESGGGLV | 2333 | GYEF | 2334 | IYPGD | 2335 | ARDGS | 2336 |
| | SASVGDRVTIT | | HSVG | | | | QFPY | | QPGGSLRLSCAA | | SRS | | GDT | | SWDW | |
| | CRSSQSIVHSV | | NTF | | | | T | | SGYEFSRSWMN | | W | | | | YFDV | |
| | GNTFLEWYQQ | | | | | | | | WVRQAPGKGLE | | | | | | | |
| | KPGKAPKLLIY | | | | | | | | WVGRIYPGDGDT | | | | | | | |
| | KVSNRFSGVP | | | | | | | | NYSGKFKGRFTIS | | | | | | | |
| | SRFSGSGSGTD | | | | | | | | ADTSKNTAYLQ | | | | | | | |
| | FTLTISSLQPE | | | | | | | | MNSLRAEDTAV | | | | | | | |
| | DFATYYCFQG | | | | | | | | YYCARDGSSWD | | | | | | | |
| | SQFPYTFGQG | | | | | | | | WYFDVWGQGTL | | | | | | | |
| | TKVEIK | | | | | | | | VTVSS | | | | | | | |
| fibro- | EIVLTQSPGTL | 2337 | QSVS | 2338 | YAS | 2339 | QQT | 2340 | EVQLLESGGGLV | 2341 | GFTF | 2342 | ISGS | 2343 | AKPF | 2344 |
| nectin | SLSPGERATLS | | SSF | | | | GRIP | | QPGGSLRLSCAA | | SSFS | | SGTT | | PYFD | |
| extra | CRASQSVSSSF | | | | | | PT | | SGFTFSSFSMSW | | | | | | Y | |
| domain- | LAWYQQKPG | | | | | | | | VRQAPGKGLEW | | | | | | | |
| B | QAPRLLIYYAS | | | | | | | | VSSISGSSGTTYY | | | | | | | |
| | SRATGIPDRFS | | | | | | | | ADSVKGRFTISR | | | | | | | |
| | GSGSGTDFTLT | | | | | | | | DNSKNTLYLQM | | | | | | | |
| | ISRLEPEDFAV | | | | | | | | NSLRAEDTAVYY | | | | | | | |
| | YYCQQTGRIPP | | | | | | | | CAKPFPYFDYWG | | | | | | | |
| | TFGQGTKVEI | | | | | | | | QGTLVTVSS | | | | | | | |
| | K | | | | | | | | | | | | | | | |
| CD3 | DIQMTQSPSSL | 2345 | SSVS | 2346 | DTS | 2347 | QQW | 2348 | QVQLVQSGAEV | 2349 | GYTF | 2350 | INPR | 2351 | ARSAY | 2352 |
| | SASVGDRVTIT | | Y | | | | SSNP | | KKPGASVKVSCK | | ISYT | | SGYT | | YDYDG | |
| | CSASSSVSYM | | | | | | PT | | ASGYTFISYTMH | | | | | | FAY | |
| | NWYQQKPGK | | | | | | | | WVRQAPGQGLE | | | | | | | |
| | APKRLIYDTSK | | | | | | | | WMGYINPRSGYT | | | | | | | |
| | LASGVPSRFSG | | | | | | | | HYNQKLKDKAT | | | | | | | |
| | SGSGTDFTLTI | | | | | | | | LTADKSASTAYM | | | | | | | |
| | SSLQPEDFATY | | | | | | | | ELSSLRSEDTAV | | | | | | | |
| | YCQQWSSNPP | | | | | | | | YYCARSAYYDY | | | | | | | |
| | TFGGGTKVEI | | | | | | | | DGFAYWGQGTL | | | | | | | |
| | K | | | | | | | | VTVSS | | | | | | | |

*Italics means immune cell target/payload (scFv arm)

In one aspect, the present disclosure provides a heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein: (a) the first polypeptide chain comprises in the N-terminal to C-terminal direction: (i) a light chain variable domain of a first immunoglobulin (VL-1) that is capable of specifically binding to a first epitope; (ii) a light chain constant domain of the first immunoglobulin (CL-1); (iii) a flexible peptide linker comprising the amino acid sequence (GGGGS)₃ (SEQ ID NO: 2506); and (iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ (SEQ ID NO: 2507) to form a single-chain variable fragment; (b) the second polypeptide comprises in the N-terminal to C-terminal direction: (i) a heavy chain variable domain of the first immunoglobulin (VH-1) that is capable of specifically binding to the first epitope; (ii) a first CH1 domain of the first immunoglobulin (CH1-1); and (iii) a first heterodimerization domain of the first immunoglobulin, wherein the first heterodimerization domain is incapable of forming a stable homodimer with another first heterodimerization domain; (c) the third polypeptide comprises in the N-terminal to C-terminal direction: (i) a heavy chain variable domain of a third immunoglobulin (VH-3) that is capable of specifically binding to a third epitope; (ii) a second CH1 domain of the third immunoglobulin (CH1-3); and (iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin; (d) the fourth polypeptide comprises in the N-terminal to C-terminal direction: (i) a light chain variable domain of the third immunoglobulin (VL-3) that is capable of specifically binding to the third epitope; (ii) a light chain constant domain of the third immunoglobulin (CL-3); (iii) a flexible peptide linker comprising the amino acid sequence (GGGGS)₃ (SEQ ID NO: 2506); and (iv) a light chain variable domain of a fourth immunoglobulin (VL-4) that is linked to a complementary heavy chain variable domain of the fourth immunoglobulin (VH-4), or a heavy chain variable domain of a fourth immunoglobulin (VH-4) that is linked to a complementary light chain variable domain of the fourth immunoglobulin (VL-4), wherein VL-4 and VH-4 are capable of specifically binding to the second epitope, and are linked together via a flexible peptide linker comprising the amino acid sequence (GGGGS)₆ (SEQ ID NO: 2507) to form a single-chain variable fragment, and wherein each of VL-1 and VL-3 independently comprises the CDR1 sequence, the CDR2 sequence and the CDR3 sequence of a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345; and/or wherein each of VH-1 and VH-3 independently comprises the CDR1 sequence, the CDR2 sequence and the CDR3 sequence of a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349.

In one aspect, the present disclosure provides a heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein: (a) the first polypeptide chain comprises in the N-terminal to C-terminal direction: (i) a light chain variable domain of a first immunoglobulin (VL-1) that is capable of specifically binding to a first epitope; (ii) a light chain constant domain of the first immunoglobulin (CL-1); (iii) a flexible peptide linker comprising the amino acid sequence (GGGGS)₃ (SEQ ID NO: 2506); and (iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein the VL-2 and VH-2 are capable of specifically binding to a second epitope, and are linked together via a flexible peptide linker comprising the amino acid sequence (GGGGS)₆ (SEQ ID NO: 2507) to form a single-chain variable fragment; (b) the second polypeptide comprises in the N-terminal to C-terminal direction: (i) a heavy chain variable domain of the first immunoglobulin (VH-1) that is capable of specifically binding to the first epitope; (ii) a first CH1 domain of the first immunoglobulin (CH1-1); and (iii) a first heterodimerization domain of the first immunoglobulin, wherein the first heterodimerization domain is incapable of forming a stable homodimer with another first heterodimerization domain; (c) the third polypeptide comprises in the N-terminal to C-terminal direction: (i) a heavy chain variable domain of a third immunoglobulin (VH-3) that is capable of specifically binding to the first epitope; (ii) a second CH1 domain of the third immunoglobulin (CH1-3); and (iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin; (d) the fourth polypeptide comprises in the N-terminal to C-terminal direction: (i) a light chain variable domain of the third immunoglobulin (VL-3) that is capable of specifically binding to the first epitope; (ii) a light chain constant domain of the third immunoglobulin (CL-3); (iii) a flexible peptide linker comprising the amino acid sequence (GGGGS)₃ (SEQ ID NO: 2506); and (iv) a light chain variable domain of a fourth immunoglobulin (VL-4) that is linked to a complementary heavy chain variable domain of the fourth immunoglobulin (VH-4), or a heavy chain variable domain of a fourth immunoglobulin (VH-4) that is linked to a complementary light chain variable domain of the fourth immunoglobulin (VL-4), wherein the VL-4 and VH-4 are capable of specifically binding to a third epitope, and are linked together via a flexible peptide linker comprising the amino acid sequence (GGGGS)₆ (SEQ ID NO: 2507) to form a single-chain variable fragment, and wherein each of VL-2 and VL-4 independently comprises the CDR1 sequence, the CDR2 sequence and the CDR3 sequence of a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345; and/or wherein each of VH-2 and VH-4 independently comprises the CDR1 sequence, the CDR2 sequence and the CDR3 sequence of a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349.

In another aspect, the present disclosure provides a heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein: (a) the first polypeptide chain comprises in the N-terminal to C-terminal direction: (i) a light chain variable domain of a first immunoglobulin (VL-1) that is capable of specifically binding to a first epitope; (ii) a light chain constant domain of the first immunoglobulin (CL-1); (iii) a flexible peptide linker comprising the amino acid sequence (GGGGS)₃ (SEQ ID NO: 2506); and (iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ (SEQ ID NO: 2507) to form a single-chain variable fragment; (b) the second polypeptide comprises in the N-terminal to C-terminal direction: (i) a heavy chain variable domain of the first immunoglobulin (VH-1) that is capable of specifically binding to the first epitope; (ii) a first CH1 domain of the first immunoglobulin (CH1-1); and (iii) a first heterodimerization domain of the first immunoglobulin, wherein the first heterodimerization domain is incapable of forming a stable homodimer with another first heterodimerization domain; (c) the third polypeptide comprises in the N-terminal to C-terminal direction: (i) a heavy chain variable domain of a third immunoglobulin (VH-3) that is capable of specifically binding to a third epitope; (ii) a second CH1 domain of the third immunoglobulin (CH1-3); and (iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin; (d) the fourth polypeptide comprises in the N-terminal to C-terminal direction: (i) a light chain variable domain of the third immunoglobulin (VL-3) that is capable of specifically binding to the third epitope; (ii) a light chain constant domain of the third immunoglobulin (CL-3); (iii) a flexible peptide linker comprising the amino acid sequence (GGGGS)₃ (SEQ ID NO: 2506); and (iv) a light chain variable domain of a fourth immunoglobulin (VL-4) that is linked to a complementary heavy chain variable domain of the fourth immunoglobulin (VH-4), or a heavy chain variable domain of a fourth immunoglobulin (VH-4) that is linked to a complementary light chain variable domain of the fourth immunoglobulin (VL-4), wherein VL-4 and VH-4 are capable of specifically binding to the fourth epitope, and are linked together via a flexible peptide linker comprising the amino acid sequence (GGGGS)₆ (SEQ ID NO: 2507) to form a single-chain variable fragment; and wherein each of VL-1 and VL-3 independently comprises the CDR1 sequence, the CDR2 sequence and the CDR3 sequence of a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345; and/or wherein each of VH-1 and VH-3 independently comprises the CDR1 sequence, the CDR2 sequence and the CDR3 sequence of a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349; and/or wherein each of VL-2 and VL-4 independently comprises the CDR1 sequence, the CDR2 sequence and the CDR3 sequence of a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345; and/or wherein each of VH-2 and VH-4 independently comprises the CDR1 sequence, the CDR2 sequence and the CDR3 sequence of a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349.

In another aspect, the present disclosure provides a heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein: (a) the first polypeptide chain comprises in the N-terminal to C-terminal direction: (i) a light chain variable domain of a first immunoglobulin (VL-1) that is capable of specifically binding to a first epitope; (ii) a light chain constant domain of the first immunoglobulin (CL-1); (iii) a flexible peptide linker comprising the amino acid sequence (GGGGS)₃ (SEQ ID NO: 2506); and (iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ (SEQ ID NO: 2507) to form a single-chain variable fragment; (b) the second polypeptide comprises in the N-terminal to C-terminal direction: (i) a heavy chain variable domain of the first immunoglobulin (VH-1) that is capable of specifically binding to the first epitope; (ii) a first CH1 domain of the first immunoglobulin (CH1-1); and (iii) a first heterodimerization domain of the first immunoglobulin, wherein the first heterodimerization domain is incapable of forming a stable homodimer with another first heterodimerization domain; (c) the third polypeptide comprises in the N-terminal to C-terminal direction: (i) a heavy chain variable domain of a third immunoglobulin (VH-3) that is capable of specifically binding to the first epitope; (ii) a second CH1 domain of the third immunoglobulin (CH1-3); and (iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin; (d) the fourth polypeptide comprises in the N-terminal to C-terminal direction: (i) a light chain variable domain of the third immunoglobulin (VL-3) that is capable of specifically binding to the first epitope; and (ii) a light chain constant domain of the third immunoglobulin (CL-3); and wherein VL-2 comprises the CDR1 sequence, the CDR2 sequence and the CDR3 sequence of a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345; and/or wherein VH-2 comprises the CDR1 sequence, the CDR2 sequence and the CDR3 sequence of a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349. In some embodiments, both VH-1 and VH-3 comprise the CDR1 sequence, the CDR2 sequence and the CDR3 sequence of a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349; and/or both VL-1 and VL-3 comprise the CDR1 sequence, the CDR2 sequence and the CDR3 sequence of a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345.

In yet another aspect, the present disclosure provides a heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein: (a) the first polypeptide chain comprises in the N-terminal to C-terminal direction: (i) a light chain variable domain of a first immunoglobulin (VL-1) that is capable of specifically binding to a first epitope; (ii) a light chain constant domain of the first immunoglobulin (CL-1); (iii) a flexible peptide linker comprising the amino acid sequence (GGGGS)₃ (SEQ ID NO: 2506); and (iv) a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ (SEQ ID NO: 2507) to form a single-chain variable fragment; (b) the second polypeptide comprises in the N-terminal to C-terminal direction: (i) a heavy chain variable domain of the first immunoglobulin (VH-1) that is capable of specifically binding to the first epitope; (ii) a first CH1 domain of the first immunoglobulin (CH1-1); and (iii) a first heterodimerization domain of the first immunoglobulin, wherein the first heterodimerization domain is incapable of forming a stable homodimer with another first heterodimerization domain; (c) the third polypeptide comprises in the N-terminal to C-terminal direction: (i) a heavy chain variable domain of a third immunoglobulin (VH-3) that is capable of specifically binding to a third epitope; (ii) a second CH1 domain of the third immunoglobulin (CH1-3); and (iii) a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin; (d) the fourth polypeptide comprises in the N-terminal to C-terminal direction: (i) a light chain variable domain of the third immunoglobulin (VL-3) that is capable of specifically binding to the third epitope; and (ii) a light chain constant domain of the third immunoglobulin (CL-3); and wherein each of VL-1 and VL-3 independently comprises the CDR1 sequence, the CDR2 sequence and the CDR3 sequence of a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345; and/or wherein each of VH-1 and VH-3 independently comprises the CDR1 sequence, the CDR2 sequence and the CDR3 sequence of a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349; and/or wherein VL-2 comprises the CDR1 sequence, the CDR2 sequence and the CDR3 sequence of a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345; and/or wherein VH-2 comprises the CDR1 sequence, the CDR2 sequence and the CDR3 sequence of a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349.

Additionally or alternatively, in some embodiments of the heterodimeric multispecific antibodies disclosed herein, VH-1 or VH-3 comprises an amino acid sequence that is at least 80%, at least 85%, at least 90%, at least 95%, at least 99% or 100% identical to a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349; and/or the VL-1 or VL-3 comprises an amino acid sequence that is at least 80%, at least 85%, at least 90%, at least 95%, at least 99% or 100% identical to a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345.

Additionally or alternatively, in some embodiments of the heterodimeric multispecific antibodies disclosed herein, VH-2 or VH-4 comprises an amino acid sequence that is at least 80%, at least 85%, at least 90%, at least 95%, at least 99% or 100% identical to a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349; and/or VL-2 or VL-4 comprises an amino acid sequence that is at least 80%, at least 85%, at least 90%, at least 95%, at least 99%, or 100% identical to a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345.

Additionally or alternatively, in some embodiments of the heterodimeric multispecific antibodies disclosed herein, each of VL-1 and VH-1 comprise a V_{L} amino acid sequence and a V_{H} amino acid sequence selected from the group consisting of SEQ ID NOs: 1 and 5 respectively; SEQ ID NOs: 9 and 13 respectively; SEQ ID NOs: 17 and 21 respectively; SEQ ID NOs: 25 and 29 respectively; SEQ ID NOs: 33 and 37 respectively; SEQ ID NOs: 41 and 45 respectively; SEQ ID NOs: 49 and 53 respectively; SEQ ID NOs: 57 and 61 respectively; SEQ ID NOs: 73 and 77 respectively; SEQ ID NOs: 89 and 93 respectively; SEQ ID NOs: 97 and 101 respectively; SEQ ID NOs: 105 and 109 respectively; SEQ ID NOs: 113 and 117 respectively; SEQ ID NOs: 121 and 125 respectively; SEQ ID NOs: 129 and 133 respectively; SEQ ID NOs: 145 and 149 respectively; SEQ ID NOs: 161 and 165 respectively; SEQ ID NOs: 169 and 173 respectively; SEQ ID NOs: 177 and 181 respectively; SEQ ID NOs: 193 and 197 respectively; SEQ ID NOs: 201 and 205 respectively; SEQ ID NOs: 233 and 237 respectively; SEQ ID NOs: 241 and 245 respectively; SEQ ID NOs: 257 and 261 respectively; SEQ ID NOs: 273 and 277 respectively; SEQ ID NOs: 281 and 285 respectively; SEQ ID NOs: 289 and 293 respectively; SEQ ID NOs: 297 and 301 respectively; SEQ ID NOs: 305 and 309 respectively; SEQ ID NOs: 313 and 317 respectively; SEQ ID NOs: 321 and 325 respectively; SEQ ID NOs: 329 and 333 respectively; SEQ ID NOs: 337 and 341 respectively; SEQ ID NOs: 345 and 349 respectively; SEQ ID NOs: 353 and 357 respectively; SEQ ID NOs: 361 and 365 respectively; SEQ ID NOs: 369 and 373 respectively; SEQ ID NOs: 377 and 381 respectively; SEQ ID NOs: 385 and 389 respectively; SEQ ID NOs: 393 and 397 respectively; SEQ ID NOs: 401 and 405 respectively; SEQ ID NOs: 409 and 413 respectively; SEQ ID NOs: 417 and 421 respectively; SEQ ID NOs: 425 and 429 respectively; SEQ ID NOs: 433 and 437 respectively; SEQ ID NOs: 441 and 445 respectively; SEQ ID NOs: 449 and 453 respectively; SEQ ID NOs: 457 and 461 respectively; SEQ ID NOs: 465 and 469 respectively; SEQ ID NOs: 481 and 485 respectively; SEQ ID NOs: 489 and 493 respectively; SEQ ID NOs: 497 and 501 respectively; SEQ ID NOs: 521 and 525 respectively; SEQ ID NOs: 529 and 533 respectively; SEQ ID NOs: 537 and 541 respectively; SEQ ID NOs: 545 and 549 respectively; SEQ ID NOs: 553 and 557 respectively; SEQ ID NOs: 561 and 565 respectively; SEQ ID NOs: 609 and 613 respectively; SEQ ID NOs: 617 and 621 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 729 and 733 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 769 and 773 respectively; SEQ ID NOs: 785 and 789 respectively; SEQ ID NOs: 793 and 797 respectively; SEQ ID NOs: 801 and 805 respectively; SEQ ID NOs: 809 and 813 respectively; SEQ ID NOs: 817 and 821 respectively; SEQ ID NOs: 825 and 829 respectively; SEQ ID NOs: 833 and 837 respectively; SEQ ID NOs: 841 and 845 respectively; SEQ ID NOs: 849 and 853 respectively; SEQ ID NOs: 857 and 861 respectively; SEQ ID NOs: 865 and 869 respectively; SEQ ID NOs: 873 and 877 respectively; SEQ ID NOs: 881 and 885 respectively; SEQ ID NOs: 889 and 893 respectively; SEQ ID NOs: 945 and 949 respectively; SEQ ID NOs: 953 and 957 respectively; SEQ ID NOs: 961 and 965 respectively; SEQ ID NOs: 977 and 981 respectively; SEQ ID NOs: 985 and 989 respectively; SEQ ID NOs: 993 and 997 respectively; SEQ ID NOs: 1001 and 1005 respectively; SEQ ID NOs: 1009 and 1013 respectively; SEQ ID NOs: 1017 and 1021 respectively; SEQ ID NOs: 1025 and 1029 respectively; SEQ ID NOs: 1033 and 1037 respectively; SEQ ID NOs: 1041 and 1045 respectively; SEQ ID NOs: 1065 and 1069 respectively; SEQ ID NOs: 1073 and 1077 respectively; SEQ ID NOs: 1081 and 1085 respectively; SEQ ID NOs: 1089 and 1093 respectively; SEQ ID NOs: 1097 and 1101 respectively; SEQ ID NOs: 1113 and 1117 respectively; SEQ ID NOs: 1121 and 1125 respectively; SEQ ID NOs: 1129 and 1133 respectively; SEQ ID NOs: 1137 and 1141 respectively; SEQ ID NOs: 1145 and 1149 respectively; SEQ ID NOs: 1153 and 1157 respectively; SEQ ID NOs: 1161 and 1165 respectively; SEQ ID NOs: 1169 and 1173 respectively; SEQ ID NOs: 1185 and 1189 respectively; SEQ ID NOs: 1193 and 1197 respectively; SEQ ID NOs: 1201 and 1205 respectively; SEQ ID NOs: 1209 and 1213 respectively; SEQ ID NOs: 1217 and 1221 respectively; SEQ ID NOs: 1225 and 1229 respectively; SEQ ID NOs: 1233 and 1237 respectively; SEQ ID NOs: 1241 and 1245 respectively; SEQ ID NOs: 1249 and 1253 respectively; SEQ ID NOs: 1257 and 1261 respectively; SEQ ID NOs: 1265 and 1269 respectively; SEQ ID NOs: 1273 and 1277 respectively; SEQ ID NOs: 1281 and 1285 respectively; SEQ ID NOs: 1289 and 1293 respectively; SEQ ID NOs: 1297 and 1301 respectively; SEQ ID NOs: 1305 and 1309 respectively; SEQ ID NOs: 1313 and 1317 respectively; SEQ ID NOs: 1321 and 1325 respectively; SEQ ID NOs: 1329 and 1333 respectively; SEQ ID NOs: 1337 and 1341 respectively; SEQ ID NOs: 1345 and 1349 respectively; SEQ ID NOs: 1353 and 1357 respectively; SEQ ID NOs: 1361 and 1365 respectively; SEQ ID NOs: 1369 and 1373 respectively; SEQ ID NOs: 1377 and 1381 respectively; SEQ ID NOs: 1385 and 1389 respectively; SEQ ID NOs: 1393 and 1397 respectively; SEQ ID NOs: 1401 and 1405 respectively; SEQ ID NOs: 1409 and 1413 respectively; SEQ ID NOs: 1417 and 1421 respectively; SEQ ID NOs: 1433 and 1437 respectively; SEQ ID NOs: 1441 and 1445 respectively; SEQ ID NOs: 1457 and 1461 respectively; SEQ ID NOs: 1465 and 1469 respectively; SEQ ID NOs: 1473 and 1477 respectively; SEQ ID NOs: 1481 and 1485 respectively; SEQ ID NOs: 1489 and 1493 respectively; SEQ ID NOs: 1497 and 1501 respectively; SEQ ID NOs: 1505 and 1509 respectively; SEQ ID NOs: 1513 and 1517 respectively; SEQ ID NOs: 1521 and 1525 respectively; SEQ ID NOs: 1529 and 1533 respectively; SEQ ID NOs: 1545 and 1549 respectively; SEQ ID NOs: 1553 and 1557 respectively; SEQ ID NOs: 1561 and 1565 respectively; SEQ ID NOs: 1569 and 1573 respectively; SEQ ID NOs: 1577 and 1581 respectively; SEQ ID NOs: 1585 and 1589 respectively; SEQ ID NOs: 1593 and 1597 respectively; SEQ ID NOs: 1601 and 1605 respectively; SEQ ID NOs: 1609 and 1613 respectively; SEQ ID NOs: 1617 and 1621 respectively; SEQ ID NOs: 1625 and 1629 respectively; SEQ ID NOs: 1633 and 1637 respectively; SEQ ID NOs: 1649 and 1653 respectively; SEQ ID NOs: 1657 and 1661 respectively; SEQ ID NOs: 1673 and 1677 respectively; SEQ ID NOs: 1681 and 1685 respectively; SEQ ID NOs: 1689 and 1693 respectively; SEQ ID NOs: 1697 and 1701 respectively; SEQ ID NOs: 1705 and 1709 respectively; SEQ ID NOs: 1713 and 1717 respectively; SEQ ID NOs: 1721 and 1725 respectively; SEQ ID NOs: 1729 and 1733 respectively; SEQ ID NOs: 1737 and 1741 respectively; SEQ ID NOs: 1745 and 1749 respectively; SEQ ID NOs: 1753 and 1757 respectively; SEQ ID NOs: 1761 and 1765 respectively; SEQ ID NOs: 1769 and 1773 respectively; SEQ ID NOs: 1777 and 1781 respectively; SEQ ID NOs: 1785 and 1789 respectively; SEQ ID NOs: 1793 and 1797 respectively; SEQ ID NOs: 1801 and 1805 respectively; SEQ ID NOs: 1809 and 1813 respectively; SEQ ID NOs: 1817 and 1821 respectively; SEQ ID NOs: 1833 and 1837 respectively; SEQ ID NOs: 1841 and 1845 respectively; SEQ ID NOs: 1849 and 1853 respectively; SEQ ID NOs: 1857 and 1861 respectively; SEQ ID NOs: 1865 and 1869 respectively; SEQ ID NOs: 1873 and 1877 respectively; SEQ ID NOs: 1881 and 1885 respectively; SEQ ID NOs: 1889 and 1893 respectively; SEQ ID NOs: 1913 and 1917 respectively; SEQ ID NOs: 1937 and 1941 respectively; SEQ ID NOs: 1945 and 1949 respectively; SEQ ID NOs: 1953 and 1957 respectively; SEQ ID NOs: 1961 and 1965 respectively; SEQ ID NOs: 1969 and 1973 respectively; SEQ ID NOs: 1977 and 1981 respectively; SEQ ID NOs: 1985 and 1989 respectively; SEQ ID NOs: 1993 and 1997 respectively; SEQ ID NOs: 2001 and 2005 respectively; SEQ ID NOs: 2009 and 2013 respectively; SEQ ID NOs: 2017 and 2021 respectively; SEQ ID NOs: 2025 and 2029 respectively; SEQ ID NOs: 2033 and 2037 respectively; SEQ ID NOs: 2041 and 2045 respectively; SEQ ID NOs: 2049 and 2053 respectively; SEQ ID NOs: 2057 and 2061 respectively; SEQ ID NOs: 2065 and 2069 respectively; SEQ ID NOs: 2073 and 2077 respectively; SEQ ID NOs: 2081 and 2085 respectively; SEQ ID NOs: 2089 and 2093 respectively; SEQ ID NOs: 2097 and 2101 respectively; SEQ ID NOs: 2105 and 2109 respectively; SEQ ID NOs: 2113 and 2117 respectively; SEQ ID NOs: 2121 and 2125 respectively; SEQ ID NOs: 2129 and 2133 respectively; SEQ ID NOs: 2137 and 2141 respectively; SEQ ID NOs: 2145 and 2149 respectively; SEQ ID NOs: 2153 and 2157 respectively; SEQ ID NOs: 2161 and 2165 respectively; SEQ ID NOs: 2169 and 2173 respectively; SEQ ID NOs: 2177 and 2181 respectively; SEQ ID NOs: 2185 and 2189 respectively; SEQ ID NOs: 2193 and 2197 respectively; SEQ ID NOs: 2201 and 2205 respectively; SEQ ID NOs: 2209 and 2213 respectively; SEQ ID NOs: 2217 and 2221 respectively; SEQ ID NOs: 2225 and 2229 respectively; SEQ ID NOs: 2233 and 2237 respectively; SEQ ID NOs: 2241 and 2245 respectively; SEQ ID NOs: 2249 and 2253 respectively; SEQ ID NOs: 2257 and 2261 respectively; SEQ ID NOs: 2273 and 2277 respectively; SEQ ID NOs: 2281 and 2285 respectively; SEQ ID NOs: 2305 and 2309 respectively; SEQ ID NOs: 2313 and 2317 respectively; SEQ ID NOs: 2321 and 2325 respectively; SEQ ID NOs: 2329 and 2333 respectively; SEQ ID NOs: 2337 and 2341 respectively; and SEQ ID NOs: 2345 and 2349 respectively.

Additionally or alternatively, in some embodiments of the heterodimeric multispecific antibodies disclosed herein, each of VL-3 and VH-3 comprise a V_{L} amino acid sequence and a V_{H} amino acid sequence selected from the group consisting of SEQ ID NOs: 1 and 5 respectively; SEQ ID NOs: 9 and 13 respectively; SEQ ID NOs: 17 and 21 respectively; SEQ ID NOs: 25 and 29 respectively; SEQ ID NOs: 33 and 37 respectively; SEQ ID NOs: 41 and 45 respectively; SEQ ID NOs: 49 and 53 respectively; SEQ ID NOs: 57 and 61 respectively; SEQ ID NOs: 73 and 77 respectively; SEQ ID NOs: 89 and 93 respectively; SEQ ID NOs: 97 and 101 respectively; SEQ ID NOs: 105 and 109 respectively; SEQ ID NOs: 113 and 117 respectively; SEQ ID NOs: 121 and 125 respectively; SEQ ID NOs: 129 and 133 respectively; SEQ ID NOs: 145 and 149 respectively; SEQ ID NOs: 161 and 165 respectively; SEQ ID NOs: 169 and 173 respectively; SEQ ID NOs: 177 and 181 respectively; SEQ ID NOs: 193 and 197 respectively; SEQ ID NOs: 201 and 205 respectively; SEQ ID NOs: 233 and 237 respectively; SEQ ID NOs: 241 and 245 respectively; SEQ ID NOs: 257 and 261 respectively; SEQ ID NOs: 273 and 277 respectively; SEQ ID NOs: 281 and 285 respectively; SEQ ID NOs: 289 and 293 respectively; SEQ ID NOs: 297 and 301 respectively; SEQ ID NOs: 305 and 309 respectively; SEQ ID NOs: 313 and 317 respectively; SEQ ID NOs: 321 and 325 respectively; SEQ ID NOs: 329 and 333 respectively; SEQ ID NOs: 337 and 341 respectively; SEQ ID NOs: 345 and 349 respectively; SEQ ID NOs: 353 and 357 respectively; SEQ ID NOs: 361 and 365 respectively; SEQ ID NOs: 369 and 373 respectively; SEQ ID NOs: 377 and 381 respectively; SEQ ID NOs: 385 and 389 respectively; SEQ ID NOs: 393 and 397 respectively; SEQ ID NOs: 401 and 405 respectively; SEQ ID NOs: 409 and 413 respectively; SEQ ID NOs: 417 and 421 respectively; SEQ ID NOs: 425 and 429 respectively; SEQ ID NOs: 433 and 437 respectively; SEQ ID NOs: 441 and 445 respectively; SEQ ID NOs: 449 and 453 respectively; SEQ ID NOs: 457 and 461 respectively; SEQ ID NOs: 465 and 469 respectively; SEQ ID NOs: 481 and 485 respectively; SEQ ID NOs: 489 and 493 respectively; SEQ ID NOs: 497 and 501 respectively; SEQ ID NOs: 521 and 525 respectively; SEQ ID NOs: 529 and 533 respectively; SEQ ID NOs: 537 and 541 respectively; SEQ ID NOs: 545 and 549 respectively; SEQ ID NOs: 553 and 557 respectively; SEQ ID NOs: 561 and 565 respectively; SEQ ID NOs: 609 and 613 respectively; SEQ ID NOs: 617 and 621 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 729 and 733 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 769 and 773 respectively; SEQ ID NOs: 785 and 789 respectively; SEQ ID NOs: 793 and 797 respectively; SEQ ID NOs: 801 and 805 respectively; SEQ ID NOs: 809 and 813 respectively; SEQ ID NOs: 817 and 821 respectively; SEQ ID NOs: 825 and 829 respectively; SEQ ID NOs: 833 and 837 respectively; SEQ ID NOs: 841 and 845 respectively; SEQ ID NOs: 849 and 853 respectively; SEQ ID NOs: 857 and 861 respectively; SEQ ID NOs: 865 and 869 respectively; SEQ ID NOs: 873 and 877 respectively; SEQ ID NOs: 881 and 885 respectively; SEQ ID NOs: 889 and 893 respectively; SEQ ID NOs: 945 and 949 respectively; SEQ ID NOs: 953 and 957 respectively; SEQ ID NOs: 961 and 965 respectively; SEQ ID NOs: 977 and 981 respectively; SEQ ID NOs: 985 and 989 respectively; SEQ ID NOs: 993 and 997 respectively; SEQ ID NOs: 1001 and 1005 respectively; SEQ ID NOs: 1009 and 1013 respectively; SEQ ID NOs: 1017 and 1021 respectively; SEQ ID NOs: 1025 and 1029 respectively; SEQ ID NOs: 1033 and 1037 respectively; SEQ ID NOs: 1041 and 1045 respectively; SEQ ID NOs: 1065 and 1069 respectively; SEQ ID NOs: 1073 and 1077 respectively; SEQ ID NOs: 1081 and 1085 respectively; SEQ ID NOs: 1089 and 1093 respectively; SEQ ID NOs: 1097 and 1101 respectively; SEQ ID NOs: 1113 and 1117 respectively; SEQ ID NOs: 1121 and 1125 respectively; SEQ ID NOs: 1129 and 1133 respectively; SEQ ID NOs: 1137 and 1141 respectively; SEQ ID NOs: 1145 and 1149 respectively; SEQ ID NOs: 1153 and 1157 respectively; SEQ ID NOs: 1161 and 1165 respectively; SEQ ID NOs: 1169 and 1173 respectively; SEQ ID NOs: 1185 and 1189 respectively; SEQ ID NOs: 1193 and 1197 respectively; SEQ ID NOs: 1201 and 1205 respectively; SEQ ID NOs: 1209 and 1213 respectively; SEQ ID NOs: 1217 and 1221 respectively; SEQ ID NOs: 1225 and 1229 respectively; SEQ ID NOs: 1233 and 1237 respectively; SEQ ID NOs: 1241 and 1245 respectively; SEQ ID NOs: 1249 and 1253 respectively; SEQ ID NOs: 1257 and 1261 respectively; SEQ ID NOs: 1265 and 1269 respectively; SEQ ID NOs: 1273 and 1277 respectively; SEQ ID NOs: 1281 and 1285 respectively; SEQ ID NOs: 1289 and 1293 respectively; SEQ ID NOs: 1297 and 1301 respectively; SEQ ID NOs: 1305 and 1309 respectively; SEQ ID NOs: 1313 and 1317 respectively; SEQ ID NOs: 1321 and 1325 respectively; SEQ ID NOs: 1329 and 1333 respectively; SEQ ID NOs: 1337 and 1341 respectively; SEQ ID NOs: 1345 and 1349 respectively; SEQ ID NOs: 1353 and 1357 respectively; SEQ ID NOs: 1361 and 1365 respectively; SEQ ID NOs: 1369 and 1373 respectively; SEQ ID NOs: 1377 and 1381 respectively; SEQ ID NOs: 1385 and 1389 respectively; SEQ ID NOs: 1393 and 1397 respectively; SEQ ID NOs: 1401 and 1405 respectively; SEQ ID NOs: 1409 and 1413 respectively; SEQ ID NOs: 1417 and 1421 respectively; SEQ ID NOs: 1433 and 1437 respectively; SEQ ID NOs: 1441 and 1445 respectively; SEQ ID NOs: 1457 and 1461 respectively; SEQ ID NOs: 1465 and 1469 respectively; SEQ ID NOs: 1473 and 1477 respectively; SEQ ID NOs: 1481 and 1485 respectively; SEQ ID NOs: 1489 and 1493 respectively; SEQ ID NOs: 1497 and 1501 respectively; SEQ ID NOs: 1505 and 1509 respectively; SEQ ID NOs: 1513 and 1517 respectively; SEQ ID NOs: 1521 and 1525 respectively; SEQ ID NOs: 1529 and 1533 respectively; SEQ ID NOs: 1545 and 1549 respectively; SEQ ID NOs: 1553 and 1557 respectively; SEQ ID NOs: 1561 and 1565 respectively; SEQ ID NOs: 1569 and 1573 respectively; SEQ ID NOs: 1577 and 1581 respectively; SEQ ID NOs: 1585 and 1589 respectively; SEQ ID NOs: 1593 and 1597 respectively; SEQ ID NOs: 1601 and 1605 respectively; SEQ ID NOs: 1609 and 1613 respectively; SEQ ID NOs: 1617 and 1621 respectively; SEQ ID NOs: 1625 and 1629 respectively; SEQ ID NOs: 1633 and 1637 respectively; SEQ ID NOs: 1649 and 1653 respectively; SEQ ID NOs: 1657 and 1661 respectively; SEQ ID NOs: 1673 and 1677 respectively; SEQ ID NOs: 1681 and 1685 respectively; SEQ ID NOs: 1689 and 1693 respectively; SEQ ID NOs: 1697 and 1701 respectively; SEQ ID NOs: 1705 and 1709 respectively; SEQ ID NOs: 1713 and 1717 respectively; SEQ ID NOs: 1721 and 1725 respectively; SEQ ID NOs: 1729 and 1733 respectively; SEQ ID NOs: 1737 and 1741 respectively; SEQ ID NOs: 1745 and 1749 respectively; SEQ ID NOs: 1753 and 1757 respectively; SEQ ID NOs: 1761 and 1765 respectively; SEQ ID NOs: 1769 and 1773 respectively; SEQ ID NOs: 1777 and 1781 respectively; SEQ ID NOs: 1785 and 1789 respectively; SEQ ID NOs: 1793 and 1797 respectively; SEQ ID NOs: 1801 and 1805 respectively; SEQ ID NOs: 1809 and 1813 respectively; SEQ ID NOs: 1817 and 1821 respectively; SEQ ID NOs: 1833 and 1837 respectively; SEQ ID NOs: 1841 and 1845 respectively; SEQ ID NOs: 1849 and 1853 respectively; SEQ ID NOs: 1857 and 1861 respectively; SEQ ID NOs: 1865 and 1869 respectively; SEQ ID NOs: 1873 and 1877 respectively; SEQ ID NOs: 1881 and 1885 respectively; SEQ ID NOs: 1889 and 1893 respectively; SEQ ID NOs: 1913 and 1917 respectively; SEQ ID NOs: 1937 and 1941 respectively; SEQ ID NOs: 1945 and 1949 respectively; SEQ ID NOs: 1953 and 1957 respectively; SEQ ID NOs: 1961 and 1965 respectively; SEQ ID NOs: 1969 and 1973 respectively; SEQ ID NOs: 1977 and 1981 respectively; SEQ ID NOs: 1985 and 1989 respectively; SEQ ID NOs: 1993 and 1997 respectively; SEQ ID NOs: 2001 and 2005 respectively; SEQ ID NOs: 2009 and 2013 respectively; SEQ ID NOs: 2017 and 2021 respectively; SEQ ID NOs: 2025 and 2029 respectively; SEQ ID NOs: 2033 and 2037 respectively; SEQ ID NOs: 2041 and 2045 respectively; SEQ ID NOs: 2049 and 2053 respectively; SEQ ID NOs: 2057 and 2061 respectively; SEQ ID NOs: 2065 and 2069 respectively; SEQ ID NOs: 2073 and 2077 respectively; SEQ ID NOs: 2081 and 2085 respectively; SEQ ID NOs: 2089 and 2093 respectively; SEQ ID NOs: 2097 and 2101 respectively; SEQ ID NOs: 2105 and 2109 respectively; SEQ ID NOs: 2113 and 2117 respectively; SEQ ID NOs: 2121 and 2125 respectively; SEQ ID NOs: 2129 and 2133 respectively; SEQ ID NOs: 2137 and 2141 respectively; SEQ ID NOs: 2145 and 2149 respectively; SEQ ID NOs: 2153 and 2157 respectively; SEQ ID NOs: 2161 and 2165 respectively; SEQ ID NOs: 2169 and 2173 respectively; SEQ ID NOs: 2177 and 2181 respectively; SEQ ID NOs: 2185 and 2189 respectively; SEQ ID NOs: 2193 and 2197 respectively; SEQ ID NOs: 2201 and 2205 respectively; SEQ ID NOs: 2209 and 2213 respectively; SEQ ID NOs: 2217 and 2221 respectively; SEQ ID NOs: 2225 and 2229 respectively; SEQ ID NOs: 2233 and 2237 respectively; SEQ ID NOs: 2241 and 2245 respectively; SEQ ID NOs: 2249 and 2253 respectively; SEQ ID NOs: 2257 and 2261 respectively; SEQ ID NOs: 2273 and 2277 respectively; SEQ ID NOs: 2281 and 2285 respectively; SEQ ID NOs: 2305 and 2309 respectively; SEQ ID NOs: 2313 and 2317 respectively; SEQ ID NOs: 2321 and 2325 respectively; SEQ ID NOs: 2329 and 2333 respectively; SEQ ID NOs: 2337 and 2341 respectively; and SEQ ID NOs: 2345 and 2349 respectively.

Additionally or alternatively, in some embodiments of the heterodimeric multispecific antibodies disclosed herein, each of VL-1 and VH-1 comprise a V_{L} amino acid sequence and a V_{H} amino acid sequence selected from the group consisting of SEQ ID NOs: 9 and 13 respectively; SEQ ID NOs: 49 and 53 respectively; SEQ ID NOs: 57 and 61 respectively; SEQ ID NOs: 65 and 69 respectively; SEQ ID NOs: 81 and 85 respectively; SEQ ID NOs: 153 and 157 respectively; SEQ ID NOs: 161 and 165 respectively; SEQ ID NOs: 193 and 197 respectively; SEQ ID NOs: 201 and 205 respectively; SEQ ID NOs: 273 and 277 respectively; SEQ ID NOs: 281 and 285 respectively; SEQ ID NOs: 289 and 293 respectively; SEQ ID NOs: 297 and 301 respectively; SEQ ID NOs: 305 and 309 respectively; SEQ ID NOs: 313 and 317 respectively; SEQ ID NOs: 361 and 365 respectively; SEQ ID NOs: 377 and 381 respectively; SEQ ID NOs: 393 and 397 respectively; SEQ ID NOs: 401 and 405 respectively; SEQ ID NOs: 409 and 413 respectively; SEQ ID NOs: 417 and 421 respectively; SEQ ID NOs: 425 and 429 respectively; SEQ ID NOs: 433 and 437 respectively; SEQ ID NOs: 441 and 445 respectively; SEQ ID NOs: 449 and 453 respectively; SEQ ID NOs: 457 and 461 respectively; SEQ ID NOs: 465 and 469 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 777 and 781 respectively; SEQ ID NOs: 825 and 829 respectively; SEQ ID NOs: 833 and 837 respectively; SEQ ID NOs: 841 and 845 respectively; SEQ ID NOs: 953 and 957 respectively; SEQ ID NOs: 961 and 965 respectively; SEQ ID NOs: 977 and 981 respectively; SEQ ID NOs: 993 and 997 respectively; SEQ ID NOs: 1001 and 1005 respectively; SEQ ID NOs: 1009 and 1013 respectively; SEQ ID NOs: 1017 and 1021 respectively; SEQ ID NOs: 1033 and 1037 respectively; SEQ ID NOs: 1049 and 1053 respectively; SEQ ID NOs: 1073 and 1077 respectively; SEQ ID NOs: 1081 and 1085 respectively; SEQ ID NOs: 1089 and 1093 respectively; SEQ ID NOs: 1105 and 1109 respectively; SEQ ID NOs: 1129 and 1133 respectively; SEQ ID NOs: 1137 and 1141 respectively; SEQ ID NOs: 1153 and 1157 respectively; SEQ ID NOs: 1161 and 1165 respectively; SEQ ID NOs: 1177 and 1181 respectively; SEQ ID NOs: 1225 and 1229 respectively; SEQ ID NOs: 1241 and 1245 respectively; SEQ ID NOs: 1257 and 1261 respectively; SEQ ID NOs: 1265 and 1269 respectively; SEQ ID NOs: 1297 and 1301 respectively; SEQ ID NOs: 1393 and 1397 respectively; SEQ ID NOs: 1409 and 1413 respectively; SEQ ID NOs: 1425 and 1429 respectively; SEQ ID NOs: 1441 and 1445 respectively; SEQ ID NOs: 1449 and 1453 respectively; SEQ ID NOs: 1457 and 1461 respectively; SEQ ID NOs: 1465 and 1469 respectively; SEQ ID NOs: 1473 and 1477 respectively; SEQ ID NOs: 1481 and 1485 respectively; SEQ ID NOs: 1497 and 1501 respectively; SEQ ID NOs: 1505 and 1509 respectively; SEQ ID NOs: 1513 and 1517 respectively; SEQ ID NOs: 1521 and 1525 respectively; SEQ ID NOs: 1529 and 1533 respectively; SEQ ID NOs: 1545 and 1549 respectively; SEQ ID NOs: 1553 and 1557 respectively; SEQ ID NOs: 1561 and 1565 respectively; SEQ ID NOs: 1569 and 1573 respectively; SEQ ID NOs: 1577 and 1581 respectively; SEQ ID NOs: 1585 and 1589 respectively; SEQ ID NOs: 1609 and 1613 respectively; SEQ ID NOs: 1617 and 1621 respectively; SEQ ID NOs: 1649 and 1653 respectively; SEQ ID NOs: 1657 and 1661 respectively; SEQ ID NOs: 1673 and 1677 respectively; SEQ ID NOs: 1689 and 1693 respectively; SEQ ID NOs: 1697 and 1701 respectively; SEQ ID NOs: 1705 and 1709 respectively; SEQ ID NOs: 1713 and 1717 respectively; SEQ ID NOs: 1721 and 1725 respectively; SEQ ID NOs: 1729 and 1733 respectively; SEQ ID NOs: 1745 and 1749 respectively; SEQ ID NOs: 1753 and 1757 respectively; SEQ ID NOs: 1761 and 1765 respectively; SEQ ID NOs: 1769 and 1773 respectively; SEQ ID NOs: 1777 and 1781 respectively; SEQ ID NOs: 1785 and 1789 respectively; SEQ ID NOs: 1793 and 1797 respectively; SEQ ID NOs: 1817 and 1821 respectively; SEQ ID NOs: 1833 and 1837 respectively; SEQ ID NOs: 1841 and 1845 respectively; SEQ ID NOs: 1849 and 1853 respectively; SEQ ID NOs: 1857 and 1861 respectively; SEQ ID NOs: 1865 and 1869 respectively; SEQ ID NOs: 1889 and 1893 respectively; SEQ ID NOs: 2257 and 2261 respectively; SEQ ID NOs: 2265 and 2269 respectively; SEQ ID NOs: 2281 and 2285 respectively; SEQ ID NOs: 2297 and 2301 respectively; SEQ ID NOs: 2305 and 2309 respectively; SEQ ID NOs: 2313 and 2317 respectively; SEQ ID NOs: 2321 and 2325 respectively; SEQ ID NOs: 2329 and 2333 respectively; SEQ ID NOs: 2337 and 2341 respectively; and SEQ ID NOs: 2345 and 2349 respectively.

Additionally or alternatively, in some embodiments of the heterodimeric multispecific antibodies disclosed herein, each of VL-3 and VH-3 comprise a V_{L} amino acid sequence and a V_{H} amino acid sequence selected from the group consisting of SEQ ID NOs: 9 and 13 respectively; SEQ ID NOs: 49 and 53 respectively; SEQ ID NOs: 57 and 61 respectively; SEQ ID NOs: 65 and 69 respectively; SEQ ID NOs: 81 and 85 respectively; SEQ ID NOs: 153 and 157 respectively; SEQ ID NOs: 161 and 165 respectively; SEQ ID NOs: 193 and 197 respectively; SEQ ID NOs: 201 and 205 respectively; SEQ ID NOs: 273 and 277 respectively; SEQ ID NOs: 281 and 285 respectively; SEQ ID NOs: 289 and 293 respectively; SEQ ID NOs: 297 and 301 respectively; SEQ ID NOs: 305 and 309 respectively; SEQ ID NOs: 313 and 317 respectively; SEQ ID NOs: 361 and 365 respectively; SEQ ID NOs: 377 and 381 respectively; SEQ ID NOs: 393 and 397 respectively; SEQ ID NOs: 401 and 405 respectively; SEQ ID NOs: 409 and 413 respectively; SEQ ID NOs: 417 and 421 respectively; SEQ ID NOs: 425 and 429 respectively; SEQ ID NOs: 433 and 437 respectively; SEQ ID NOs: 441 and 445 respectively; SEQ ID NOs: 449 and 453 respectively; SEQ ID NOs: 457 and 461 respectively; SEQ ID NOs: 465 and 469 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 777 and 781 respectively; SEQ ID NOs: 825 and 829 respectively; SEQ ID NOs: 833 and 837 respectively; SEQ ID NOs: 841 and 845 respectively; SEQ ID NOs: 953 and 957 respectively; SEQ ID NOs: 961 and 965 respectively; SEQ ID NOs: 977 and 981 respectively; SEQ ID NOs: 993 and 997 respectively; SEQ ID NOs: 1001 and 1005 respectively; SEQ ID NOs: 1009 and 1013 respectively; SEQ ID NOs: 1017 and 1021 respectively; SEQ ID NOs: 1033 and 1037 respectively; SEQ ID NOs: 1049 and 1053 respectively; SEQ ID NOs: 1073 and 1077 respectively; SEQ ID NOs: 1081 and 1085 respectively; SEQ ID NOs: 1089 and 1093 respectively; SEQ ID NOs: 1105 and 1109 respectively; SEQ ID NOs: 1129 and 1133 respectively; SEQ ID NOs: 1137 and 1141 respectively; SEQ ID NOs: 1153 and 1157 respectively; SEQ ID NOs: 1161 and 1165 respectively; SEQ ID NOs: 1177 and 1181 respectively; SEQ ID NOs: 1225 and 1229 respectively; SEQ ID NOs: 1241 and 1245 respectively; SEQ ID NOs: 1257 and 1261 respectively; SEQ ID NOs: 1265 and 1269 respectively; SEQ ID NOs: 1297 and 1301 respectively; SEQ ID NOs: 1393 and 1397 respectively; SEQ ID NOs: 1409 and 1413 respectively; SEQ ID NOs: 1425 and 1429 respectively; SEQ ID NOs: 1441 and 1445 respectively; SEQ ID NOs: 1449 and 1453 respectively; SEQ ID NOs: 1457 and 1461 respectively; SEQ ID NOs: 1465 and 1469 respectively; SEQ ID NOs: 1473 and 1477 respectively; SEQ ID NOs: 1481 and 1485 respectively; SEQ ID NOs: 1497 and 1501 respectively; SEQ ID NOs: 1505 and 1509 respectively; SEQ ID NOs: 1513 and 1517 respectively; SEQ ID NOs: 1521 and 1525 respectively; SEQ ID NOs: 1529 and 1533 respectively; SEQ ID NOs: 1545 and 1549 respectively; SEQ ID NOs: 1553 and 1557 respectively; SEQ ID NOs: 1561 and 1565 respectively; SEQ ID NOs: 1569 and 1573 respectively; SEQ ID NOs: 1577 and 1581 respectively; SEQ ID NOs: 1585 and 1589 respectively; SEQ ID NOs: 1609 and 1613 respectively; SEQ ID NOs: 1617 and 1621 respectively; SEQ ID NOs: 1649 and 1653 respectively; SEQ ID NOs: 1657 and 1661 respectively; SEQ ID NOs: 1673 and 1677 respectively; SEQ ID NOs: 1689 and 1693 respectively; SEQ ID NOs: 1697 and 1701 respectively; SEQ ID NOs: 1705 and 1709 respectively; SEQ ID NOs: 1713 and 1717 respectively; SEQ ID NOs: 1721 and 1725 respectively; SEQ ID NOs: 1729 and 1733 respectively; SEQ ID NOs: 1745 and 1749 respectively; SEQ ID NOs: 1753 and 1757 respectively; SEQ ID NOs: 1761 and 1765 respectively; SEQ ID NOs: 1769 and 1773 respectively; SEQ ID NOs: 1777 and 1781 respectively; SEQ ID NOs: 1785 and 1789 respectively; SEQ ID NOs: 1793 and 1797 respectively; SEQ ID NOs: 1817 and 1821 respectively; SEQ ID NOs: 1833 and 1837 respectively; SEQ ID NOs: 1841 and 1845 respectively; SEQ ID NOs: 1849 and 1853 respectively; SEQ ID NOs: 1857 and 1861 respectively; SEQ ID NOs: 1865 and 1869 respectively; SEQ ID NOs: 1889 and 1893 respectively; SEQ ID NOs: 2257 and 2261 respectively; SEQ ID NOs: 2265 and 2269 respectively; SEQ ID NOs: 2281 and 2285 respectively; SEQ ID NOs: 2297 and 2301 respectively; SEQ ID NOs: 2305 and 2309 respectively; SEQ ID NOs: 2313 and 2317 respectively; SEQ ID NOs: 2321 and 2325 respectively; SEQ ID NOs: 2329 and 2333 respectively; SEQ ID NOs: 2337 and 2341 respectively; and SEQ ID NOs: 2345 and 2349 respectively.

Additionally or alternatively, in some embodiments of the heterodimeric multispecific antibodies disclosed herein, each of VL-2 and VH-2 comprise a V_{L} amino acid sequence and a V_{H} amino acid sequence selected from the group consisting of SEQ ID NOs: 17 and 21 respectively; SEQ ID NOs: 25 and 29 respectively; SEQ ID NOs: 33 and 37 respectively; SEQ ID NOs: 41 and 45 respectively; SEQ ID NOs: 121 and 125 respectively; SEQ ID NOs: 137 and 141 respectively; SEQ ID NOs: 169 and 173 respectively; SEQ ID NOs: 177 and 181 respectively; SEQ ID NOs: 185 and 189 respectively; SEQ ID NOs: 193 and 197 respectively; SEQ ID NOs: 201 and 205 respectively; SEQ ID NOs: 209 and 213 respectively; SEQ ID NOs: 217 and 221 respectively; SEQ ID NOs: 225 and 229 respectively; SEQ ID NOs: 233 and 237 respectively; SEQ ID NOs: 241 and 245 respectively; SEQ ID NOs: 249 and 253 respectively; SEQ ID NOs: 257 and 261 respectively; SEQ ID NOs: 265 and 269 respectively; SEQ ID NOs: 321 and 325 respectively; SEQ ID NOs: 329 and 333 respectively; SEQ ID NOs: 337 and 341 respectively; SEQ ID NOs: 393 and 397 respectively; SEQ ID NOs: 401 and 405 respectively; SEQ ID NOs: 409 and 413 respectively; SEQ ID NOs: 473 and 477 respectively; SEQ ID NOs: 481 and 485 respectively; SEQ ID NOs: 489 and 493 respectively; SEQ ID NOs: 497 and 501 respectively; SEQ ID NOs: 505 and 509 respectively; SEQ ID NOs: 513 and 517 respectively; SEQ ID NOs: 545 and 549 respectively; SEQ ID NOs: 553 and 557 respectively; SEQ ID NOs: 561 and 565 respectively; SEQ ID NOs: 569 and 573 respectively; SEQ ID NOs: 577 and 581 respectively; SEQ ID NOs: 585 and 589 respectively; SEQ ID NOs: 593 and 597 respectively; SEQ ID NOs: 601 and 605 respectively; SEQ ID NOs: 625 and 629 respectively; SEQ ID NOs: 633 and 637 respectively; SEQ ID NOs: 641 and 645 respectively; SEQ ID NOs: 649 and 653 respectively; SEQ ID NOs: 657 and 661 respectively; SEQ ID NOs: 665 and 669 respectively; SEQ ID NOs: 673 and 677 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 729 and 733 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 769 and 773 respectively; SEQ ID NOs: 785 and 789 respectively; SEQ ID NOs: 793 and 797 respectively; SEQ ID NOs: 801 and 805 respectively; SEQ ID NOs: 809 and 813 respectively; SEQ ID NOs: 817 and 821 respectively; SEQ ID NOs: 849 and 853 respectively; SEQ ID NOs: 857 and 861 respectively; SEQ ID NOs: 865 and 869 respectively; SEQ ID NOs: 873 and 877 respectively; SEQ ID NOs: 881 and 885 respectively; SEQ ID NOs: 889 and 893 respectively; SEQ ID NOs: 897 and 901 respectively; SEQ ID NOs: 905 and 909 respectively; SEQ ID NOs: 913 and 917 respectively; SEQ ID NOs: 921 and 925 respectively; SEQ ID NOs: 929 and 933 respectively; SEQ ID NOs: 937 and 941 respectively; SEQ ID NOs: 945 and 949 respectively; SEQ ID NOs: 969 and 973 respectively; SEQ ID NOs: 977 and 981 respectively; SEQ ID NOs: 1009 and 1013 respectively; SEQ ID NOs: 1057 and 1061 respectively; SEQ ID NOs: 1537 and 1541 respectively; SEQ ID NOs: 1569 and 1573 respectively; SEQ ID NOs: 1601 and 1605 respectively; SEQ ID NOs: 1641 and 1645 respectively; SEQ ID NOs: 1665 and 1669 respectively; SEQ ID NOs: 1825 and 1829 respectively; SEQ ID NOs: 1865 and 1869 respectively; SEQ ID NOs: 1897 and 1901 respectively; SEQ ID NOs: 1905 and 1909 respectively; SEQ ID NOs: 1913 and 1917 respectively; SEQ ID NOs: 1921 and 1925 respectively; SEQ ID NOs: 1929 and 1933 respectively; SEQ ID NOs: 2265 and 2269 respectively; SEQ ID NOs: 2281 and 2285 respectively; 2289 and 2293 respectively; 2329 and 2333 respectively; and SEQ ID NOs: 2345 and 2349, respectively.

Additionally or alternatively, in some embodiments of the heterodimeric multispecific antibodies disclosed herein, each of VL-4 and VH-4 comprise a V_{L} amino acid sequence and a V_{H} amino acid sequence selected from the group consisting of SEQ ID NOs: 17 and 21 respectively; SEQ ID NOs: 25 and 29 respectively; SEQ ID NOs: 33 and 37 respectively; SEQ ID NOs: 41 and 45 respectively; SEQ ID NOs: 121 and 125 respectively; SEQ ID NOs: 137 and 141 respectively; SEQ ID NOs: 169 and 173 respectively; SEQ ID NOs: 177 and 181 respectively; SEQ ID NOs: 185 and 189 respectively; SEQ ID NOs: 193 and 197 respectively; SEQ ID NOs: 201 and 205 respectively; SEQ ID NOs: 209 and 213 respectively; SEQ ID NOs: 217 and 221 respectively; SEQ ID NOs: 225 and 229 respectively; SEQ ID NOs: 233 and 237 respectively; SEQ ID NOs: 241 and 245 respectively; SEQ ID NOs: 249 and 253 respectively; SEQ ID NOs: 257 and 261 respectively; SEQ ID NOs: 265 and 269 respectively; SEQ ID NOs: 321 and 325 respectively; SEQ ID NOs: 329 and 333 respectively; SEQ ID NOs: 337 and 341 respectively; SEQ ID NOs: 393 and 397 respectively; SEQ ID NOs: 401 and 405 respectively; SEQ ID NOs: 409 and 413 respectively; SEQ ID NOs: 473 and 477 respectively; SEQ ID NOs: 481 and 485 respectively; SEQ ID NOs: 489 and 493 respectively; SEQ ID NOs: 497 and 501 respectively; SEQ ID NOs: 505 and 509 respectively; SEQ ID NOs: 513 and 517 respectively; SEQ ID NOs: 545 and 549 respectively; SEQ ID NOs: 553 and 557 respectively; SEQ ID NOs: 561 and 565 respectively; SEQ ID NOs: 569 and 573 respectively; SEQ ID NOs: 577 and 581 respectively; SEQ ID NOs: 585 and 589 respectively; SEQ ID NOs: 593 and 597 respectively; SEQ ID NOs: 601 and 605 respectively; SEQ ID NOs: 625 and 629 respectively; SEQ ID NOs: 633 and 637 respectively; SEQ ID NOs: 641 and 645 respectively; SEQ ID NOs: 649 and 653 respectively; SEQ ID NOs: 657 and 661 respectively; SEQ ID NOs: 665 and 669 respectively; SEQ ID NOs: 673 and 677 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 729 and 733 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 769 and 773 respectively; SEQ ID NOs: 785 and 789 respectively; SEQ ID NOs: 793 and 797 respectively; SEQ ID NOs: 801 and 805 respectively; SEQ ID NOs: 809 and 813 respectively; SEQ ID NOs: 817 and 821 respectively; SEQ ID NOs: 849 and 853 respectively; SEQ ID NOs: 857 and 861 respectively; SEQ ID NOs: 865 and 869 respectively; SEQ ID NOs: 873 and 877 respectively; SEQ ID NOs: 881 and 885 respectively; SEQ ID NOs: 889 and 893 respectively; SEQ ID NOs: 897 and 901 respectively; SEQ ID NOs: 905 and 909 respectively; SEQ ID NOs: 913 and 917 respectively; SEQ ID NOs: 921 and 925 respectively; SEQ ID NOs: 929 and 933 respectively; SEQ ID NOs: 937 and 941 respectively; SEQ ID NOs: 945 and 949 respectively; SEQ ID NOs: 969 and 973 respectively; SEQ ID NOs: 977 and 981 respectively; SEQ ID NOs: 1009 and 1013 respectively; SEQ ID NOs: 1057 and 1061 respectively; SEQ ID NOs: 1537 and 1541 respectively; SEQ ID NOs: 1569 and 1573 respectively; SEQ ID NOs: 1601 and 1605 respectively; SEQ ID NOs: 1641 and 1645 respectively; SEQ ID NOs: 1665 and 1669 respectively; SEQ ID NOs: 1825 and 1829 respectively; SEQ ID NOs: 1865 and 1869 respectively; SEQ ID NOs: 1897 and 1901 respectively; SEQ ID NOs: 1905 and 1909 respectively; SEQ ID NOs: 1913 and 1917 respectively; SEQ ID NOs: 1921 and 1925 respectively; SEQ ID NOs: 1929 and 1933 respectively; SEQ ID NOs: 2265 and 2269 respectively; SEQ ID NOs: 2281 and 2285 respectively; 2289 and 2293 respectively; 2329 and 2333 respectively; and SEQ ID NOs: 2345 and 2349, respectively.

Additionally or alternatively, in some embodiments of the heterodimeric multispecific antibodies disclosed herein, the first immunoglobulin or the third immunoglobulin binds to a cell surface antigen selected from the group consisting of a2b b3 (Glycoprotein IIb/IIIa), a4, a4b7, a4b7+aEb7, a5, Activin receptor type-2B, ALK1, Alpha-synuclein, amyloid beta, APP, AXL, Blood Group A, CAIX, CCL-2, CD105 (endoglin), CD115 (CSF1R), CD116a (CSF2Ra), CD123, CD152 (CTLA4), CD184 (CXCR4), CD19, CD192 (CCR2), CD194 (CCR4), CD195 (CCR5), CD20, CD200, CD22, CD221 (IGF1R), CD248, CD25, CD257 (BAFF), CD26, CD262 (DR5), CD276 (B7H3), CD3, CD30 (TNFRSF8), CD319 (SLAMF7), CD33, CD332 (FGFR2), CD350 (FZD10), CD37, CD371 (CLEC12A), CD38, CD4, CD49b (a2), CD51 (a5), CD52, CD56, CD61 (a4b3), CD70, CD73 (NTSE), CD74, CEA, Claudin-18.2, cMET, CRLR, DLL3, DLL4, DNA/histone (H1) complex, EGFR, EpCAM, EGFR-HER3, EGFRvIII, EphA3, ERGT(GalNAc) Tn Antigen, FLT1, FOLR1, frizzled family receptor (FZD), Lewis Y, Lewis X, GCGR, GD2, GD2 α-acetyl, GD3, GM1, GM1 fucosyl, GM2, GPA33, GPNMB, GUCY2C, HER2, HER3, HGFR (cMET), IgHe, IGLF2, Kallikreins, LINGO1, LOXL2, Ly6/PLAUR domain-containing protein 3, MADCAM1, MAG, Mesothelin, MT1-MMP (MMP14), MUC1, Mucin SAC, NaPi2b, NeuGc-GM3, notch, NOTCH2/NOTCH3 receptors, oxLDL, P-selectin, PCSK9, PDGFRA, PDGFRa, phosphatidylserine, polysialic acid, PSMA, PVRL4, RGMA, CD240D Blood group D antigen, root plate-specific spondin 3, serum amyloid P component, STEAP-1, TACSTD2, TGFb, TWEAKR, TYRP1, VEGFR2, VSIR, CD171 (L1CAM), CD19, CD47, pMHC[NY-ESO1], pMHC[MART1], pMHC[MAGEA1], pMHC[Tyrosinase], pMHC[gp100], pMHC[MUC1], pMHC[tax], pMHC[WT-1], pMHC[EBNA-1], pMHC[LMP2], pMHC[hTERT], GPC3, CD80, CD23, and fibronectin extra domain-B. The first immunoglobulin and the third immunoglobulin may bind to the same epitope on a target cell or two different epitopes on a target cell. In some embodiments, the target cell is a cancer cell.

Additionally or alternatively, in some embodiments of the heterodimeric multispecific antibodies disclosed herein, the second immunoglobulin or the fourth immunoglobulin bind to an epitope on a white blood cell, a monocyte, a lymphocyte, a granulocyte, a macrophage, a T cell, a NK cell, a B cell, a NKT cell, an ILC, or neutrophil.

In any of the above embodiments of the heterodimeric multispecific antibodies disclosed herein, the second immunoglobulin or the fourth immunoglobulin bind to an antigen selected from the group consisting of Dabigatran, a4, a4b7, a4b7+aEb7, a5, AXL, BnDOTA, CD11a (LFA-1), CD3, CD4, CD8, CD16, CD19, CD22, CD23, CD25, CD28, CD30 (TNFRSF8), CD33, CD38, CD40, CD40L, CD47, CD49b (a2), CD54 (ICAM-1), CD56, CD74, CD80, CD115 (CSF1R), CD116a (CSF2Ra), CD123, CD134 (OX40), CD137 (41BB), CD152 (CTLA4), CD184 (CXCR4), CD192 (CCR2), CD194 (CCR4), CD195 (CCR5), CD223 (LAG-3), CD252 (OX40L), CD254 (RANKL), CD262 (DR5), CD27, CD200, CD221 (IGF1R), CD248, CD274 (PD-L1), CD275 (ICOS-L), CD278 (ICOS), CD279 (PD-1), CD319 (SLAMF7), CD371 (CLEC12A), MADCAM1, MT1-MMP (MMP14), NKG2A, NRP1,TIGIT, VSIR, KIRDL1/2/3, and KIR2DL2. The second immunoglobulin and the fourth immunoglobulin may bind to the same epitope or different epitopes on a white blood cell, a monocyte, a lymphocyte, a granulocyte, a macrophage, a T cell, a NK cell, a B cell, a NKT cell, an ILC, or neutrophil. In some embodiments, the second immunoglobulin binds CD3 and the fourth immunoglobulin binds an immune cell receptor selected from the group consisting of CD4, CD8, CD25, CD28, CTLA4, OX40, ICOS, PD-1, PD-L1, 41BB, CD2, CD69, and CD45. In other embodiments, the second immunoglobulin binds CD16 and the fourth immunoglobulin binds an immune cell receptor selected from the group consisting of CD56, NKG2D, and KIRDL1/2/3. In certain embodiments, the fourth immunoglobulin binds to an agent selected from the group consisting of a cytokine, a nucleic acid, a hapten, a small molecule, a radionuclide, an immunotoxin, a vitamin, a peptide, a lipid, a carbohydrate, biotin, digoxin, or any conjugated variants thereof.

Additionally or alternatively, in some embodiments of the heterodimeric multispecific antibodies disclosed herein, the first immunoglobulin and the third immunoglobulin bind to their respective epitopes with a monovalent affinity or an effective affinity between about 100 nM to about 100 pM. In certain embodiments, the first immunoglobulin and the third immunoglobulin bind to cell surface epitopes that are between 60 and 120 angstroms apart.

Additionally or alternatively, in some embodiments of the heterodimeric multispecific antibodies disclosed herein, the first immunoglobulin and the third immunoglobulin bind to their respective epitopes with a monovalent affinity or an effective affinity that is less than 100 pM. In certain embodiments, the first immunoglobulin and the third immunoglobulin bind to cell surface epitopes that are up to 180 angstroms apart.

Additionally or alternatively, in some embodiments of the heterodimeric multispecific antibodies disclosed herein, the first heterodimerization domain of the first immunoglobulin and/or the second heterodimerization domain of the third immunoglobulin is a CH2-CH3 domain and has an isotype selected from the group consisting of IgG1, IgG2, IgG3, IgG4, IgA1, IgA2, IgM, IgD, and IgE. Non-limiting examples of constant region sequences include:

Human IgD constant region, Uniprot: P01880
(SEQ ID NO: 2381)
APTKAPDVFPIISGCRHPKDNSPVVLACLITGYHPTSVTVTWYMGTQSQP
QRTFPEIQRRDSYYMTSSQLSTPLQQWRQGEYKCVVQHTASKSKKEIFRW
PESPKAQASSVPTAQPQAEGSLAKATTAPATTRNTGRGGEEKKKEKEKEE
QEERETKTPECPSHTQPLGVYLLTPAVQDLWLRDKATFTCFVVGSDLKDA
HLTWEVAGKVPTGGVEEGLLERHSNGSQSQHSRLTLPRSLWNAGTSVTCT
LNHPSLPPQRLMALREPAAQAPVKLSLNLLASSDPPEAASWLLCEVSGFS
PPNILLMWLEDQREVNTSGFAPARPPPQPGSTTFWAWSVLRVPAPPSPQP
ATYTCVVSHEDSRTLLNASRSLEVSYVTDHGPMK
Human IgG1 constant region, Uniprot: P01857
(SEQ ID NO: 2382)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Human IgG2 constant region, Uniprot: P01859
(SEQ ID NO: 2383)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDISVEWESNGQPENNYKTTPPMLDSDGSFFLYSKLTVDKSRWQQGN
VFSCSVMHEALHNHYTQKSLSLSPGK
Human IgG3 constant region, Uniprot: P01860
(SEQ ID NO: 2384)
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK
Human IgM constant region, Uniprot: P01871
(SEQ ID NO: 2385)
GSASAPTLFPLVSCENSPSDTSSVAVGCLAQDFLPDSITLSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLGQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLRESATIT
CLVTGFSPADVFVQWMQRGQPLSPEKYVTSAPMPEPQAPGRYFAHSILTV
SEEEWNTGETYTCVAHEALPNRVTERTVDKSTGKPTLYNVSLVMSDTAGT
CY
Human IgG4 constant region, Uniprot: P01861
(SEQ ID NO: 2386)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
Human IgA1 constant region, Uniprot: P01876
(SEQ ID NO: 2387)
ASPTSPKVFPLSLCSTQPDGNVVIACLVQGFFPQEPLSVTWSESGQGVTA
RNFPPSQDASGDLYTTSSQLTLPATQCLAGKSVTCHVKHYTNPSQDVTVP
CPVPSTPPTPSPSTPPTPSPSCCHPRLSLHRPALEDLLLGSEANLTCTLT
GLRDASGVTFTWTPSSGKSAVQGPPERDLCGCYSVSSVLPGCAEPWNHGK
TFTCTAAYPESKTPLTATLSKSGNTFRPEVHLLPPPSEELALNELVTLTC
LARGFSPKDVLVRWLQGSQELPREKYLTWASRQEPSQGTTTFAVTSILRV
AAEDWKKGDTFSCMVGHEALPLAFTQKTIDRLAGKPTHVNVSVVMAEVDG
TCY
Human IgA2 constant region, Uniprot: P01877
(SEQ ID NO: 2388)
ASPTSPKVFPLSLDSTPQDGNVVVACLVQGFFPQEPLSVTWSESGQNVTA
RNFPPSQDASGDLYTTSSQLTLPATQCPDGKSVTCHVKHYTNPSQDVTVP
CPVPPPPPCCHPRLSLHRPALEDLLLGSEANLTCTLTGLRDASGATFTWT
PSSGKSAVQGPPERDLCGCYSVSSVLPGCAQPWNHGETFTCTAAHPELKT
PLTANITKSGNTFRPEVHLLPPPSEELALNELVTLTCLARGFSPKDVLVR
WLQGSQELPREKYLTWASRQEPSQGTTTFAVTSILRVAAEDWKKGDTFSC
MVGHEALPLAFTQKTIDRMAGKPTHVNVSVVMAEVDGTCY
Human Ig kappa constant region, Uniprot: P01834
(SEQ ID NO: 2389)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC

In some embodiments, the immunoglobulin-related compositions of the present technology comprise a heavy chain constant region that is at least 80%, at least 85%, at least 90%, at least 95%, at least 99%, or is 100% identical to SEQ ID NOS: 2381-2388. Additionally or alternatively, in some embodiments, the immunoglobulin-related compositions of the present technology comprise a light chain constant region that is at least 80%, at least 85%, at least 90%, at least 95%, at least 99%, or is 100% identical to SEQ ID NO: 2389.

Additionally or alternatively, in some embodiments of the heterodimeric multispecific antibodies disclosed herein, the first heterodimerization domain of the first immunoglobulin and/or the second heterodimerization domain of the third immunoglobulin is an IgG1 constant region comprising one or more amino acid substitutions selected from the group consisting of N297A and K322A. Additionally or alternatively, in some embodiments of the heterodimeric multispecific antibodies disclosed herein, the first heterodimerization domain of the first immunoglobulin is a CH2-CH3 domain comprising a K409R mutation and the second heterodimerization domain of the third immunoglobulin is a CH2-CH3 domain comprising a F405L mutation.

Also disclosed herein are recombinant nucleic acid sequences encoding any of the antibodies described herein. In another aspect, the present technology provides a host cell or vector expressing any nucleic acid sequence encoding any immunoglobulin-related composition described herein.

In some embodiments, the immunoglobulin-related compositions of the present technology are chimeric, humanized, or monoclonal. The immunoglobulin-related compositions of the present technology can further be recombinantly fused to a heterologous polypeptide at the N or C terminus or chemically conjugated (including covalently and non-covalently conjugations) to polypeptides or other compositions. For example, the immunoglobulin-related compositions of the present technology can be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

In any of the above embodiments of the immunoglobulin-related compositions of the present technology, the HDTVS antibody may be optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof. For a chemical bond or physical bond, a functional group on the immunoglobulin-related composition typically associates with a functional group on the agent. Alternatively, a functional group on the agent associates with a functional group on the immunoglobulin-related composition.

The functional groups on the agent and immunoglobulin-related composition can associate directly. For example, a functional group (e.g., a sulfhydryl group) on an agent can associate with a functional group (e.g., sulfhydryl group) on an immunoglobulin-related composition to form a disulfide. Alternatively, the functional groups can associate through a cross-linking agent (i.e., linker). Some examples of cross-linking agents are described below. The cross-linker can be attached to either the agent or the immunoglobulin-related composition. The number of agents or immunoglobulin-related compositions in a conjugate is also limited by the number of functional groups present on the other. For example, the maximum number of agents associated with a conjugate depends on the number of functional groups present on the immunoglobulin-related composition. Alternatively, the maximum number of immunoglobulin-related compositions associated with an agent depends on the number of functional groups present on the agent.

In yet another embodiment, the conjugate comprises one immunoglobulin-related composition associated to one agent. In one embodiment, a conjugate comprises at least one agent chemically bonded (e.g., conjugated) to at least one immunoglobulin-related composition. The agent can be chemically bonded to an immunoglobulin-related composition by any method known to those in the art. For example, a functional group on the agent may be directly attached to a functional group on the immunoglobulin-related composition. Some examples of suitable functional groups include, for example, amino, carboxyl, sulfhydryl, maleimide, isocyanate, isothiocyanate and hydroxyl.

The agent may also be chemically bonded to the immunoglobulin-related composition by means of cross-linking agents, such as dialdehydes, carbodiimides, dimaleimides, and the like. Cross-linking agents can, for example, be obtained from Pierce Biotechnology, Inc., Rockford, Ill. The Pierce Biotechnology, Inc. web-site can provide assistance. Additional cross-linking agents include the platinum cross-linking agents described in U.S. Pat. Nos. 5,580,990; 5,985,566; and 6,133,038 of Kreatech Biotechnology, B.V., Amsterdam, The Netherlands.

Alternatively, the functional group on the agent and immunoglobulin-related composition can be the same. Homobifunctional cross-linkers are typically used to cross-link identical functional groups. Examples of homobifunctional cross-linkers include EGS (i.e., ethylene glycol bis[succinimidylsuccinate]), DSS (i.e., disuccinimidyl suberate), DMA (i.e., dimethyl adipimidate.2HCl), DTSSP (i.e., 3,3′-dithiobis[sulfosuccinimidylpropionate])), DPDPB (i.e., 1,4-di-[3′-(2′-pyridyldithio)-propionamido]butane), and BMH (i.e., bis-maleimidohexane). Such homobifunctional cross-linkers are also available from Pierce Biotechnology, Inc.

In other instances, it may be beneficial to cleave the agent from the immunoglobulin-related composition. The web-site of Pierce Biotechnology, Inc. described above can also provide assistance to one skilled in the art in choosing suitable cross-linkers which can be cleaved by, for example, enzymes in the cell. Thus the agent can be separated from the immunoglobulin-related composition. Examples of cleavable linkers include SMPT (i.e., 4-succinimidyloxycarbonyl-methyl-a-[2-pyridyldithio]toluene), Sulfo-LC-SPDP (i.e., sulfosuccinimidyl 6-(3-[2-pyridyldithio]-propionamido)hexanoate), LC-SPDP (i.e., succinimidyl 6-(3-[2-pyridyldithio]-propionamido)hexanoate), Sulfo-LC-SPDP (i.e., sulfosuccinimidyl 6-(3-[2-pyridyldithio]-propionamido)hexanoate), SPDP (i.e., N-succinimidyl 3-[2-pyridyldithio]-propionamidohexanoate), and AEDP (i.e., 3-[(2-aminoethyl)dithio]propionic acid HCl).

In another embodiment, a conjugate comprises at least one agent physically bonded with at least one immunoglobulin-related composition. Any method known to those in the art can be employed to physically bond the agents with the immunoglobulin-related compositions. For example, the immunoglobulin-related compositions and agents can be mixed together by any method known to those in the art. The order of mixing is not important. For instance, agents can be physically mixed with immunoglobulin-related compositions by any method known to those in the art. For example, the immunoglobulin-related compositions and agents can be placed in a container and agitated, by for example, shaking the container, to mix the immunoglobulin-related compositions and agents.

The immunoglobulin-related compositions can be modified by any method known to those in the art. For instance, the immunoglobulin-related composition may be modified by means of cross-linking agents or functional groups, as described above.

Heterodimerization. The present technology is dependent on heterodimerization of two IgG-scFv half-molecules through mutations in the heterodimerization domains using techniques known in the art. Any heterodimerization approach where the hinge domain is kept in place may be employed, provided that sufficient antibody stability is achieved.

Heterodimerization of CH2-CH3 domains. Formation of a heterodimeric trivalent/tetravalent multispecific antibody molecule of the present technology requires the interaction of four different polypeptide chains. Such interactions are difficult to achieve with efficiency within a single cell recombinant production system, due to the many variants of potential chain mispairings. One solution to increase the probability of mispairings, is to engineer “knobs-into-holes” type mutations into the desired polypeptide chain pairs. Such mutations favor heterodimerization over homodimerization. For example, with respect to Fc-Fc-interactions, an amino acid substitution (preferably a substitution with an amino acid comprising a bulky side group forming a ‘knob’, e.g., tryptophan) can be introduced into the CH2 or CH3 domain such that steric interference will prevent interaction with a similarly mutated domain and will obligate the mutated domain to pair with a domain into which a complementary, or accommodating mutation has been engineered, i.e., ‘the hole’ (e.g., a substitution with glycine). Such sets of mutations can be engineered into a pair of polypeptides that are included within the heterodimeric trivalent/tetravalent molecule (e.g., the second polypeptide chain and the third polypeptide chain), and further, engineered into any portion of the polypeptides chains of said pair. Methods of protein engineering to favor heterodimerization over homodimerization are well known in the art, in particular with respect to the engineering of immunoglobulin-like molecules, and are encompassed herein (see e.g., Ridgway et al., 1996, Protein Engr. 9:617-621, Atwell et al., 1997, J. Mol. Biol. 270: 26-35, and Xie et al., 2005, J. Immunol. Methods 296:95-101; each of which is hereby incorporated herein by reference in its entirety).

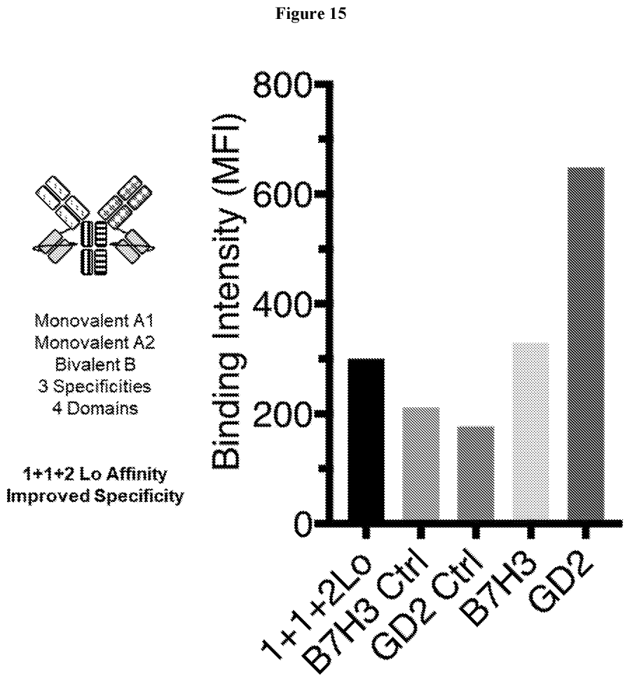

The design of variant Fc heterodimers from wildtype homodimers is illustrated by the concept of positive and negative design in the context of protein engineering by balancing stability vs. specificity, where mutations are introduced with the goal of driving heterodimer formation over homodimer formation when the polypeptides are expressed in cell culture conditions. Negative design strategies maximize unfavorable interactions for the formation of homodimers, by either introducing bulky sidechains on one chain and small sidechains on the opposite, for example the knobs-into-holes strategy developed by Genentech (Ridgway J B, Presta L G, Carter P. Protein Eng. 1996 July; 9(7):617-21; Atwell S, Ridgway J B, Wells J A, Carter P. J Mol. Biol. 270(1):26-35 (1997))), or by electrostatic engineering that leads to repulsion of homodimer formation, for example the electrostatic steering strategy developed by Amgen (Gunaskekaran K, et al. JBC 285 (25): 19637-19646 (2010)). In these two examples, negative design asymmetric point mutations are introduced into the wild-type CH3 domain to drive heterodimer formation. Other heterodimerization approaches are described in US 20120149876 (e.g., at Tables 1, 6 and 7), and US 20140294836 (e.g., at FIGS. 15A-B, 16A-B, and 17). Methods for engineering Fc heterodimers using electrostatic steering are described in detail in U.S. Pat. No. 8,592,562.

In some embodiments of the HDTVS antibodies disclosed herein, the second polypeptide chain and the third polypeptide chain comprise a first CH2-CH3 domain and a second CH2-CH3 domain respectively, wherein the first CH2-CH3 domain and the second CH2-CH3 domain comprise amino acid modifications selected from the group consisting of: T366Y and Y407T respectively; F405A and T394W respectively; Y349C/T366S/L368A/Y407V and S354C/T366W respectively; K409D/K392D and D399K respectively; T366S/L368A/Y407V and T366W respectively; K409D/K392D and D399K/E356K respectively; L351Y/Y407A and T366A/K409F respectively; L351Y/Y407A and T366V/K409F respectively; Y407A and T366A/K409F respectively; D399R/S400R/Y407A and T366A/K409F/K392E/T411E respectively; L351Y/F405A/Y407V and T394W respectively; L351Y/F405A/Y407V and T366L respectively; F405A/Y407V and T366I/K392M/T394W respectively; F405A/Y407V and T366L/K392M/T394W respectively; F405A/Y407V and T366L/T394W respectively; F405A/Y407V and T366I/T394W respectively; and K409R and F405L respectively.

In some embodiments of the HDTVS antibodies disclosed herein, the second polypeptide chain and the third polypeptide chain comprise a first CH2-CH3 domain and a second CH2-CH3 domain respectively, wherein the first CH2-CH3 domain comprises an amino acid modification at position F405 and amino acid modifications L351Y and Y407V, and the second CH2-CH3 domain comprises amino acid modification T394W. In some embodiments, the amino acid modification at position F405 is F405A, F4051, F405M, F405T, F4055, F405V or F405W.

In some embodiments of the HDTVS antibodies disclosed herein, the second polypeptide chain and the third polypeptide chain comprise a first CH2-CH3 domain and a second CH2-CH3 domain respectively, wherein the first CH2-CH3 domain comprises amino acid modifications at positions L351 and Y407, and the second CH2-CH3 domain comprises an amino acid modification at position T366 and amino acid modification K409F. In some embodiments, the amino acid modification at position L351 is L351Y, L3511, L351D, L351R or L351F. In some embodiments, the amino acid modification at position Y407 is Y407A, Y407V or Y4075. In certain embodiments, the amino acid modification at position T366 is T366A, T366I, T366L, T366M, T366Y, T366S, T366C, T366V or T366W.

In some embodiments of the HDTVS antibodies disclosed herein, the second polypeptide chain and the third polypeptide chain comprise a first CH2-CH3 domain and a second CH2-CH3 domain respectively, wherein the first CH2-CH3 domain or the second CH2-CH3 domain comprises an amino acid modification at positions K392, T411, T366, L368 or 5400. The amino acid modification at position K392 may be K392V, K392M, K392R, K392L, K392F or K392E. The amino acid modification at position T411 may be T411N, T411R, T411Q, T411K, T411D, T411E or T411W. The amino acid modification at position 5400 may be S400E, 5400D, 5400R or S400K. The amino acid modification at position T366 may be T366A, T366I, T366L, T366M, T366Y, T366S, T366C, T366V or T366W. The amino acid modification at position L368 may be L368D, L368R, L368T, L368M, L368V, L368F, L368S and L368A.

In some embodiments of the HDTVS antibodies disclosed herein, the second polypeptide chain and the third polypeptide chain comprise a first CH2-CH3 domain and a second CH2-CH3 domain respectively, wherein the first CH2-CH3 domain comprises amino acid modifications L351Y and Y407A and the second CH2-CH3 domain comprises amino acid modifications T366A and K409F, and optionally wherein the first CH2-CH3 domain or the second CH2-CH3 domain comprises one or more amino acid modifications at position T411, D399, 5400, F405, N390, or K392. The amino acid modification at position T411 may be T411N, T411R, T411Q, T411K, T411D, T411E or T411W. The amino acid modification at position D399 may be D399R, D399W, D399Y or D399K. The amino acid modification at position 5400 may be S400E, 5400D, 5400R, or S400K. The amino acid modification at position F405 may be F4051, F405M, F405T, F4055, F405V or F405W. The amino acid modification at position N390 may be N390R, N390K or N390D. The amino acid modification at position K392 may be K392V, K392M, K392R, K392L, K392F or K392E.

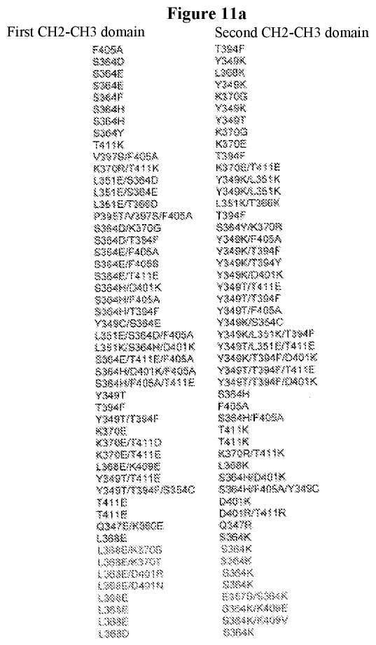

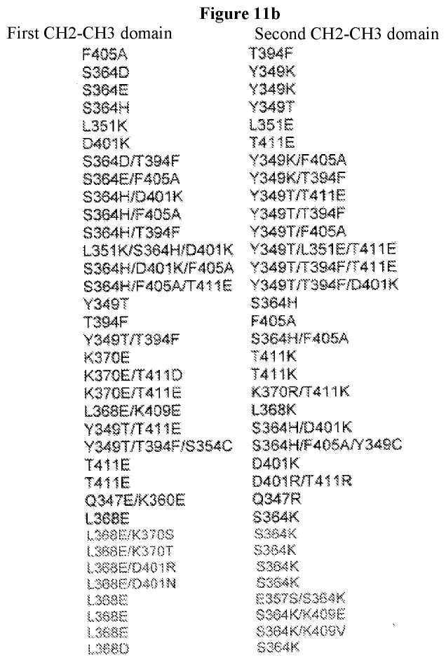

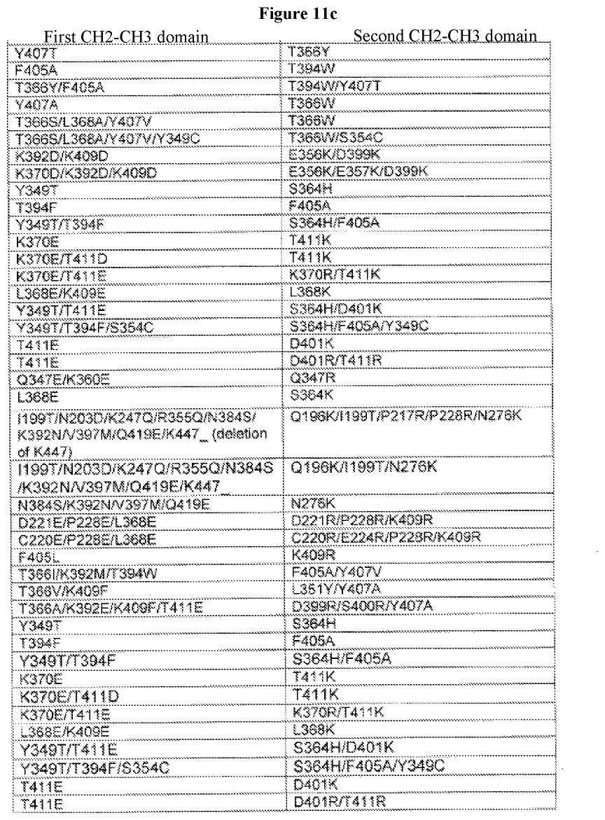

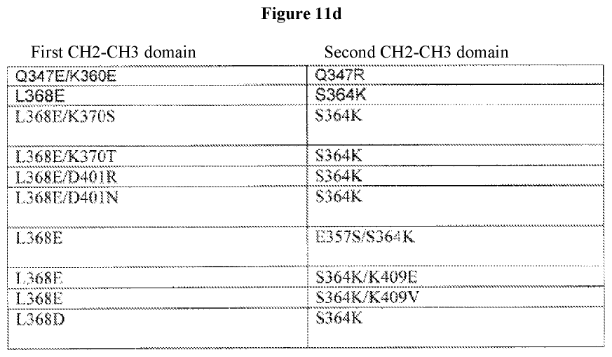

In some embodiments of the HDTVS antibodies disclosed herein, the second polypeptide chain and the third polypeptide chain comprise a first CH2-CH3 domain and a second CH2-CH3 domain respectively, wherein the first CH2-CH3 domain and the second CH2-CH3 domain comprise a set of amino acid modifications as shown in FIG. 11a. In some embodiments of the HDTVS antibodies disclosed herein, the second polypeptide chain and the third polypeptide chain comprise a first CH2-CH3 domain and a second CH2-CH3 domain respectively, wherein the first CH2-CH3 domain and the second CH2-CH3 domain comprise a set of amino acid modifications as shown in FIG. 11b. In some embodiments of the HDTVS antibodies disclosed herein, the second polypeptide chain and the third polypeptide chain comprise a first CH2-CH3 domain and a second CH2-CH3 domain respectively, wherein the first CH2-CH3 domain and the second CH2-CH3 domain comprise a set of amino acid modifications as shown in FIG. 11c. In some embodiments of the HDTVS antibodies disclosed herein, the second polypeptide chain and the third polypeptide chain comprise a first CH2-CH3 domain and a second CH2-CH3 domain respectively, wherein the first CH2-CH3 domain and the second CH2-CH3 domain comprise a set of amino acid modifications as shown in FIG. 11d. In some embodiments of the HDTVS antibodies disclosed herein, the second polypeptide chain and the third polypeptide chain comprise a first CH2-CH3 domain and a second CH2-CH3 domain respectively, wherein the first CH2-CH3 domain and the second CH2-CH3 domain comprise a set of amino acid modifications as shown in FIG. 11e.

Other Fc Modifications. In some embodiments, the heterodimeric trivalent/tetravalent multispecific antibodies of the present technology comprise a variant Fc region, wherein said variant Fc region comprises at least one amino acid modification relative to a wild-type Fc region (or the parental Fc region), such that said molecule has an altered affinity for an Fc receptor (e.g., an FcγR), provided that said variant Fc region does not have a substitution at positions that make a direct contact with Fc receptor based on crystallographic and structural analysis of Fc-Fc receptor interactions such as those disclosed by Sondermann et al., Nature, 406:267-273 (2000). Examples of positions within the Fc region that make a direct contact with an Fc receptor such as an FcγR, include amino acids 234-239 (hinge region), amino acids 265-269 (B/C loop), amino acids 297-299 (C7E loop), and amino acids 327-332 (F/G) loop.

In some embodiments, a heterodimeric trivalent/tetravalent multispecific antibody of the present technology has an altered affinity for activating and/or inhibitory receptors, and includes a variant Fc region with one or more amino acid modifications, wherein said one or more amino acid modification is a N297 substitution with alanine, or a K322 substitution with alanine.

Glycosylation Modifications. In some embodiments, heterodimeric trivalent/tetravalent multispecific antibodies of the present technology have an Fc region with variant glycosylation as compared to a parent Fc region. In some embodiments, variant glycosylation includes the absence of fucose; in some embodiments, variant glycosylation results from expression in GnT1-deficient CHO cells.

In some embodiments, the antibodies of the present technology, may have a modified glycosylation site relative to an appropriate reference antibody that binds to an antigen of interest, without altering the functionality of the antibody, e.g., binding activity to the antigen. As used herein, “glycosylation sites” include any specific amino acid sequence in an antibody to which an oligosaccharide (i.e., carbohydrates containing two or more simple sugars linked together) will specifically and covalently attach.

Oligosaccharide side chains are typically linked to the backbone of an antibody via either N- or O-linkages. N-linked glycosylation refers to the attachment of an oligosaccharide moiety to the side chain of an asparagine residue. O-linked glycosylation refers to the attachment of an oligosaccharide moiety to a hydroxyamino acid, e.g., serine, threonine. For example, an Fc-glycoform that lacks certain oligosaccharides including fucose and terminal N-acetylglucosamine may be produced in special CHO cells and exhibit enhanced ADCC effector function.

In some embodiments, the carbohydrate content of an immunoglobulin-related composition disclosed herein is modified by adding or deleting a glycosylation site. Methods for modifying the carbohydrate content of antibodies are well known in the art and are included within the present technology, see, e.g., U.S. Pat. No. 6,218,149; EP 0359096B1; U.S. Patent Publication No. US 2002/0028486; International Patent Application Publication WO 03/035835; U.S. Patent Publication No. 2003/0115614; U.S. Pat. Nos. 6,218,149; 6,472,511; all of which are incorporated herein by reference in their entirety. In some embodiments, the carbohydrate content of an antibody (or relevant portion or component thereof) is modified by deleting one or more endogenous carbohydrate moieties of the antibody. In certain embodiments, the present technology includes deleting the glycosylation site of the Fc region of an antibody, by modifying position 297 from asparagine to alanine. Such antibodies lack Fc effector function. In some embodiments, nonspecific FcR-dependent binding in normal tissues is eliminated or reduced (e.g., via N297A mutation in Fc region, which results in aglycosylation).

Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example DI N-acetylglucosaminyltransferase III (GnTIII), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al., 1999, Nat. Biotechnol. 17: 176-180; Davies et al., 2001, Biotechnol. Bioeng. 74:288-294; Shields et al., 2002, 1 Biol. Chem. 277:26733-26740; Shinkawa et al., 2003, J Biol. Chem. 278:3466-3473; U.S. Pat. No. 6,602,684; U.S. patent application Ser. No. 10/277,370; U.S. patent application Ser. No. 10/113,929; International Patent Application Publications WO 00/61739A1; WO 01/292246A1; WO 02/311140A1; WO 02/30954A1; POTILLEGENT™ technology (Biowa, Inc. Princeton, N.J.); GLYCOMAB™ glycosylation engineering technology (GLYCART biotechnology AG, Zurich, Switzerland); each of which is incorporated herein by reference in its entirety. See, e.g., International Patent Application Publication WO 00/061739; U.S. Patent Application Publication No. 2003/0115614; Okazaki et al., 2004, JMB, 336: 1239-49.

### A. Methods of Preparing Heterodimeric Trivalent/Tetravalent Multispecific Antibodies of the Present Technology

General Overview. The heterodimeric trivalent/tetravalent multispecific antibodies of the present disclosure can be produced using a variety of methods well known in the art, including de novo protein synthesis and recombinant expression of nucleic acids encoding the binding proteins. Initially, a target antigen is chosen to which an antibody of the present technology can be raised. For example, in some embodiments, an antibody may be raised against a full-length target protein, or to a portion of the target protein. Techniques for generating antibodies directed to such target polypeptides are well known to those skilled in the art. Examples of such techniques include, for example, but are not limited to, those involving display libraries, xeno or human mice, hybridomas, and the like.

Generally, an antibody is obtained from an originating species. More particularly, the nucleic acid or amino acid sequence of the variable portion of the light chain, heavy chain or both, of an originating species antibody having specificity for a target antigen is obtained. An originating species is any species which was useful to generate the antibody of the present technology or library of antibodies, e.g., rat, mouse, rabbit, chicken, monkey, human, and the like.

Phage or phagemid display technologies are useful techniques to derive the antibodies of the present technology. Techniques for generating and cloning monoclonal antibodies are well known to those skilled in the art. Expression of sequences encoding antibodies of the present technology, can be carried out in E. coli.

Due to the degeneracy of nucleic acid coding sequences, other sequences which encode substantially the same amino acid sequences as those of the naturally occurring proteins may be used in the practice of the present technology. These include, but are not limited to, nucleic acid sequences including all or portions of the nucleic acid sequences encoding the above polypeptides, which are altered by the substitution of different codons that encode a functionally equivalent amino acid residue within the sequence, thus producing a silent change. It is appreciated that the nucleotide sequence of an immunoglobulin according to the present technology tolerates sequence homology variations of up to 25% as calculated by standard methods (“Current Methods in Sequence Comparison and Analysis,” Macromolecule Sequencing and Synthesis, Selected Methods and Applications, pp. 127-149, 1998, Alan R. Liss, Inc.) so long as such a variant yields an operative antibody which recognizes a target of interest. For example, one or more amino acid residues within a polypeptide sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Also included within the scope of the present technology are proteins or fragments or derivatives thereof which are differentially modified during or after translation, e.g., by glycosylation, proteolytic cleavage, linkage to an antibody molecule or other cellular ligands, etc. Additionally, an immunoglobulin encoding nucleic acid sequence can be mutated in vitro or in vivo to create and/or destroy translation, initiation, and/or termination sequences or to create variations in coding regions and/or form new restriction endonuclease sites or destroy pre-existing ones, to facilitate further in vitro modification. Any technique for mutagenesis known in the art can be used, including but not limited to in vitro site directed mutagenesis, J. Biol. Chem. 253:6551, use of Tab linkers (Pharmacia), and the like.

Monoclonal Antibody. In one embodiment of the present technology, the heterodimeric trivalent/tetravalent multispecific antibody is a monoclonal antibody. For example, in some embodiments, the heterodimeric trivalent/tetravalent multispecific monoclonal antibody may be a human or a mouse heterodimeric trivalent/tetravalent multispecific monoclonal antibody. For preparation of monoclonal antibodies directed towards a target molecule of interest, any technique that provides for the production of antibody molecules by continuous cell line culture can be utilized. Such techniques include, but are not limited to, the hybridoma technique (See, e.g., Kohler & Milstein, 1975. Nature 256: 495-497); the trioma technique; the human B-cell hybridoma technique (See, e.g., Kozbor, et al., 1983. Immunol. Today 4: 72) and the EBV hybridoma technique to produce human monoclonal antibodies (See, e.g., Cole, et al., 1985. In: MONOCLONAL ANTIBODIES AND CANCER THERAPY, Alan R. Liss, Inc., pp. 77-96). Human monoclonal antibodies can be utilized in the practice of the present technology and can be produced by using human hybridomas (See, e.g., Cote, et al., 1983. Proc. Natl. Acad. Sci. USA 80: 2026-2030) or by transforming human B-cells with Epstein Barr Virus in vitro (See, e.g., Cole, et al., 1985. In: MONOCLONAL ANTIBODIES AND CANCER THERAPY, Alan R. Liss, Inc., pp. 77-96). For example, a population of nucleic acids that encode regions of antibodies can be isolated. PCR utilizing primers derived from sequences encoding conserved regions of antibodies is used to amplify sequences encoding portions of antibodies from the population and then DNAs encoding polypeptide chains of the heterodimeric trivalent/tetravalent multispecific antibodies or fragments thereof, such as variable domains, are reconstructed from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the target molecule of interest. Alternatively, hybridomas expressing heterodimeric trivalent/tetravalent multispecific monoclonal antibodies can be prepared by immunizing a subject and then isolating hybridomas from the subject's spleen using routine methods. See, e.g., Milstein et al., (Galfre and Milstein, Methods Enzymol (1981) 73: 3-46). Screening the hybridomas using standard methods will produce monoclonal antibodies of varying specificity (i.e., for different epitopes) and affinity. A selected monoclonal antibody with the desired properties, e.g., binding to a target antigen, can be used as expressed by the hybridoma, it can be bound to a molecule such as polyethylene glycol (PEG) to alter its properties, or a cDNA encoding it can be isolated, sequenced and manipulated in various ways. Synthetic dendromeric trees can be added to reactive amino acid side chains, e.g., lysine, to enhance the immunogenic properties of a target protein. Also, CPG-dinucleotide techniques can be used to enhance the immunogenic properties of the target protein. Other manipulations include substituting or deleting particular amino acyl residues that contribute to instability of the antibody during storage or after administration to a subject, and affinity maturation techniques to improve affinity of the antibody towards its target antigen.

Hybridoma Technique. In some embodiments, the antibody of the present technology is a heterodimeric trivalent/tetravalent multispecific monoclonal antibody produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, N.Y., 349 (1988); Hammerling et al., Monoclonal Antibodies And T-Cell Hybridomas, 563-681 (1981). Other methods for producing hybridomas and monoclonal antibodies are well known to those of skill in the art.

Phage Display Technique. As noted above, the antibodies of the present technology can be produced through the application of recombinant DNA and phage display technology. For example, heterodimeric trivalent/tetravalent multi specific antibodies, can be prepared using various phage display methods known in the art. In phage display methods, functional antibody domains are displayed on the surface of a phage particle which carries polynucleotide sequences encoding them. Phages with a desired binding property are selected from a repertoire or combinatorial antibody library (e.g., human or murine) by selecting directly with an antigen, typically an antigen bound or captured to a solid surface or bead. Phages used in these methods are typically filamentous phage including fd and M13 with Fab, Fv or disulfide stabilized Fv antibody domains that are recombinantly fused to either the phage gene III or gene VIII protein. In addition, methods can be adapted for the construction of Fab expression libraries (See, e.g., Huse, et al., Science 246: 1275-1281, 1989) to allow rapid and effective identification of monoclonal Fab fragments with the desired specificity for a target antigen, e.g., a target polypeptide or derivatives, fragments, analogs or homologs thereof. Other examples of phage display methods that can be used to make the antibodies of the present technology include those disclosed in Huston et al., Proc. Natl. Acad. Sci U.S.A., 85: 5879-5883, 1988; Chaudhary et al., Proc. Natl. Acad. Sci U.S.A., 87: 1066-1070, 1990; Brinkman et al., J. Immunol. Methods 182: 41-50, 1995; Ames et al., J. Immunol. Methods 184: 177-186, 1995; Kettleborough et al., Eur. J Immunol. 24: 952-958, 1994; Persic et al., Gene 187: 9-18, 1997; Burton et al., Advances in Immunology 57: 191-280, 1994; PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743. Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12: 864-869, 1992; and Sawai et al., AJRI 34: 26-34, 1995; and Better et al., Science 240: 1041-1043, 1988.

Generally, hybrid antibodies or hybrid antibody fragments that are cloned into a display vector can be selected against the appropriate antigen in order to identify variants that maintain good binding activity, because the antibody or antibody fragment will be present on the surface of the phage or phagemid particle. See, e.g., Barbas III et al., Phage Display, A Laboratory Manual (Cold Spring Harbor Laboratory Press, Cold Spring Harbor, N.Y., 2001). However, other vector formats could be used for this process, such as cloning the antibody fragment library into a lytic phage vector (modified T7 or Lambda Zap systems) for selection and/or screening.

Single-Chain Fvs. The heterodimeric trivalent/tetravalent multispecific antibody of the present technology comprises two single-chain Fvs. According to the present technology, techniques can be adapted for the production of single-chain antibodies specific to a target antigen (See, e.g., U.S. Pat. No. 4,946,778). Examples of techniques which can be used to produce single-chain Fvs and antibodies of the present technology include those described in U.S. Pat. Nos. 4,946,778 and 5,258,498; Huston et al., Methods in Enzymology, 203: 46-88, 1991; Shu, L. et al., Proc. Natl. Acad. Sci. USA, 90: 7995-7999, 1993; and Skerra et al., Science 240: 1038-1040, 1988.

Chimeric and Humanized Antibodies. In one embodiment, the heterodimeric trivalent/tetravalent multispecific antibody of the present technology is chimeric. In one embodiment, the heterodimeric trivalent/tetravalent multispecific antibody of the present technology is humanized. In one embodiment of the present technology, the donor and acceptor antibodies are monoclonal antibodies from different species. For example, the acceptor antibody is a human antibody (to minimize its antigenicity in a human), in which case the resulting CDR-grafted antibody is termed a “humanized” antibody.

Recombinant heterodimeric trivalent/tetravalent multispecific antibodies, such as chimeric and humanized monoclonal antibodies, comprising both human and non-human portions, can be made using standard recombinant DNA techniques, and are within the scope of the present technology. For some uses, including in vivo use of the heterodimeric trivalent/tetravalent multispecific antibody of the present technology in humans as well as use of these agents in in vitro detection assays, it is possible to use chimeric or humanized heterodimeric trivalent/tetravalent multispecific antibodies. Such chimeric and humanized monoclonal antibodies can be produced by recombinant DNA techniques known in the art. Such useful methods include, e.g., but are not limited to, methods described in International Application No. PCT/US86/02269; U.S. Pat. No. 5,225,539; European Patent No. 184187; European Patent No. 171496; European Patent No. 173494; PCT International Publication No. WO 86/01533; U.S. Pat. Nos. 4,816,567; 5,225,539; European Patent No. 125023; Better, et al., 1988. Science 240: 1041-1043; Liu, et al., 1987. Proc. Natl. Acad. Sci. USA 84: 3439-3443; Liu, et al., 1987. J Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314: 446-449; Shaw, et al., 1988. J Natl. Cancer Inst. 80: 1553-1559; Morrison (1985) Science 229: 1202-1207; Oi, et al. (1986) BioTechniques 4: 214; Jones, et al., 1986. Nature 321: 552-525; Verhoeyan, et al., 1988. Science 239: 1534; Morrison, Science 229: 1202, 1985; Oi et al., BioTechniques 4: 214, 1986; Gillies et al., J. Immunol. Methods, 125: 191-202, 1989; U.S. Pat. No. 5,807,715; and Beidler, et al., 1988. J. Immunol. 141: 4053-4060. For example, antibodies can be humanized using a variety of techniques including CDR-grafting (EP 0 239 400; WO 91/09967; U.S. Pat. Nos. 5,530,101; 5,585,089; 5,859,205; 6,248,516; EP460167), veneering or resurfacing (EP 0 592 106; EP 0 519 596; Padlan E. A., Molecular Immunology, 28: 489-498, 1991; Studnicka et al., Protein Engineering 7: 805-814, 1994; Roguska et al., PNAS 91: 969-973, 1994), and chain shuffling (U.S. Pat. No. 5,565,332). In one embodiment, a cDNA encoding a murine heterodimeric trivalent/tetravalent multispecific monoclonal antibody is digested with a restriction enzyme selected specifically to remove the sequence encoding the Fc constant region, and the equivalent portion of a cDNA encoding a human Fc constant region is substituted (See Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 125,023; Better et al. (1988) Science 240: 1041-1043; Liu et al. (1987) Proc. Natl. Acad. Sci. USA 84: 3439-3443; Liu et al. (1987) J Immunol 139: 3521-3526; Sun et al. (1987) Proc. Natl. Acad. Sci. USA 84: 214-218; Nishimura et al. (1987) Cancer Res 47: 999-1005; Wood et al. (1985) Nature 314: 446-449; and Shaw et al. (1988) J. Natl. Cancer Inst. 80: 1553-1559; U.S. Pat. Nos. 6,180,370; 6,300,064; 6,696,248; 6,706,484; 6,828,422.

In one embodiment, the present technology provides the construction of humanized heterodimeric trivalent/tetravalent multispecific antibodies that are unlikely to induce a human anti-mouse antibody (hereinafter referred to as “HAMA”) response, while still having an effective antibody effector function. As used herein, the terms “human” and “humanized”, in relation to antibodies, relate to any antibody which is expected to elicit a therapeutically tolerable weak immunogenic response in a human subject. In one embodiment, the present technology provides for a humanized heterodimeric trivalent/tetravalent multispecific antibody comprising both heavy chain and light chain polypeptides.

CDR Antibodies. In some embodiments, the heterodimeric trivalent/tetravalent multispecific antibody of the present technology is a CDR antibody. Generally the donor and acceptor antibodies used to generate the heterodimeric trivalent/tetravalent multispecific CDR antibody are monoclonal antibodies from different species; typically the acceptor antibody is a human antibody (to minimize its antigenicity in a human), in which case the resulting CDR-grafted antibody is termed a “humanized” antibody. The graft may be of a single CDR (or even a portion of a single CDR) within a single V_{H} or V_{L} of the acceptor antibody, or can be of multiple CDRs (or portions thereof) within one or both of the V_{H} and V_{L}. Frequently, all three CDRs in all variable domains of the acceptor antibody will be replaced with the corresponding donor CDRs, though one need replace only as many as necessary to permit adequate binding of the resulting CDR-grafted antibody to the target antigen. Methods for generating CDR-grafted and humanized antibodies are taught by Queen et al. U.S. Pat. Nos. 5,585,089; 5,693,761; 5,693,762; and Winter U.S. Pat. No. 5,225,539; and EP 0682040. Methods useful to prepare V_{H} and V_{L} polypeptides are taught by Winter et al., U.S. Pat. Nos. 4,816,397; 6,291,158; 6,291,159; 6,291,161; 6,545,142; EP 0368684; EP0451216; and EP0120694.

After selecting suitable framework region candidates from the same family and/or the same family member, either or both the heavy and light chain variable regions are produced by grafting the CDRs from the originating species into the hybrid framework regions. Assembly of hybrid antibodies or hybrid antibody fragments having hybrid variable chain regions with regard to either of the above aspects can be accomplished using conventional methods known to those skilled in the art. For example, DNA sequences encoding the hybrid variable domains described herein (i.e., frameworks based on the target species and CDRs from the originating species) can be produced by oligonucleotide synthesis and/or PCR. The nucleic acid encoding CDR regions can also be isolated from the originating species antibodies using suitable restriction enzymes and ligated into the target species framework by ligating with suitable ligation enzymes. Alternatively, the framework regions of the variable chains of the originating species antibody can be changed by site-directed mutagenesis.

Since the hybrids are constructed from choices among multiple candidates corresponding to each framework region, there exist many combinations of sequences which are amenable to construction in accordance with the principles described herein. Accordingly, libraries of hybrids can be assembled having members with different combinations of individual framework regions. Such libraries can be electronic database collections of sequences or physical collections of hybrids.

This process typically does not alter the acceptor antibody's FRs flanking the grafted CDRs. However, one skilled in the art can sometimes improve antigen binding affinity of the resulting heterodimeric trivalent/tetravalent multispecific CDR-grafted antibody by replacing certain residues of a given FR to make the FR more similar to the corresponding FR of the donor antibody. Suitable locations of the substitutions include amino acid residues adjacent to the CDR, or which are capable of interacting with a CDR (See, e.g., U.S. Pat. No. 5,585,089, especially columns 12-16). Or one skilled in the art can start with the donor FR and modify it to be more similar to the acceptor FR or a human consensus FR. Techniques for making these modifications are known in the art. Particularly if the resulting FR fits a human consensus FR for that position, or is at least 90% or more identical to such a consensus FR, doing so may not increase the antigenicity of the resulting modified heterodimeric trivalent/tetravalent multispecific CDR-grafted antibody significantly compared to the same antibody with a fully human FR.

Expression of Recombinant Heterodimeric Trivalent/Tetravalent Multispecific Antibodies. The desired nucleic acid sequences can be produced by recombinant methods (e.g., PCR mutagenesis of an earlier prepared variant of the desired polynucleotide) or by solid-phase DNA synthesis. Because of the degeneracy of the genetic code, a variety of nucleic acid sequences encode each immunoglobulin amino acid sequence, and the present disclosure includes all nucleic acids encoding the binding proteins described herein, which are suitable for use in accordance with the present disclosure.

Once the nucleotide sequence of the heterodimeric trivalent/tetravalent multispecific antibodies are determined, the nucleotide sequence may be manipulated using methods well known in the art, e.g., recombinant DNA techniques, site directed mutagenesis, PCR, etc. (see, for example, the techniques described in Sambrook et al., 2001, Molecular Cloning, A Laboratory Manual, 3rd Ed., Cold Spring Harbor Laboratory, Cold Spring Harbor, N.Y.; and Ausubel et al., eds., 1998, Current Protocols in Molecular Biology, John Wiley & Sons, NY, which are both incorporated by reference herein in their entireties), to generate, for example, antibodies having a different amino acid sequence, for example by generating amino acid substitutions, deletions, and/or insertions. In one embodiment, human libraries or any other libraries available in the art, can be screened by standard techniques known in the art, to clone the nucleic acids encoding the heterodimeric trivalent/tetravalent multispecific antibodies of the present disclosure.

As noted above, the antibodies of the present technology can be produced through the application of recombinant DNA technology. Recombinant polynucleotide constructs encoding a heterodimeric trivalent/tetravalent multispecific antibody of the present technology typically include an expression control sequence operably-linked to the coding sequences of heterodimeric trivalent/tetravalent multispecific antibody chains, including naturally-associated or heterologous promoter regions. As such, another aspect of the technology includes vectors containing one or more nucleic acid sequences encoding a heterodimeric trivalent/tetravalent multispecific antibody of the present technology. Methods which are well known to those skilled in the art can be used to construct expression vectors containing the coding sequences for the molecules of the present disclosure and appropriate transcriptional and translational control signals. These methods include, for example, in vitro recombinant DNA techniques, synthetic techniques, and in vivo genetic recombination. See, for example, the techniques described in Sambrook et al., 1990, Molecular Cloning, A Laboratory Manual, 2d Ed., Cold Spring Harbor Laboratory, Cold Spring Harbor, N.Y. and Ausubel et al. eds., 1998, Current Protocols in Molecular Biology, John Wiley & Sons, NY. For recombinant expression of one or more of the polypeptides of the present technology, the nucleic acid containing all or a portion of the nucleotide sequence encoding the heterodimeric trivalent/tetravalent multispecific antibody is inserted into an appropriate cloning vector, or an expression vector (i.e., a vector that contains the necessary elements for the transcription and translation of the inserted polypeptide coding sequence) by recombinant DNA techniques well known in the art and as detailed below. Methods for producing diverse populations of vectors have been described by Lerner et al., U.S. Pat. Nos. 6,291,160 and 6,680,192.

In general, expression vectors useful in recombinant DNA techniques are often in the form of plasmids. In the present disclosure, “plasmid” and “vector” can be used interchangeably as the plasmid is the most commonly used form of vector. However, the present technology is intended to include such other forms of expression vectors that are not technically plasmids, such as viral vectors (e.g., replication defective retroviruses, adenoviruses and adeno-associated viruses), which serve equivalent functions. Such viral vectors permit infection of a subject and expression of a construct in that subject. In some embodiments, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences encoding the heterodimeric trivalent/tetravalent multispecific antibody, and the collection and purification of the heterodimeric trivalent/tetravalent multispecific antibody, e.g., cross-reacting heterodimeric trivalent/tetravalent multispecific antibodies. See generally, U.S. 2002/0199213. These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences. Vectors can also encode signal peptide, e.g., pectate lyase, useful to direct the secretion of extracellular antibody fragments. See U.S. Pat. No. 5,576,195.

The recombinant expression vectors of the present technology comprise a nucleic acid encoding a protein having binding properties to a molecule of interest and in a form suitable for expression of the nucleic acid in a host cell, which means that the recombinant expression vectors include one or more regulatory sequences, selected on the basis of the host cells to be used for expression that is operably-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, e.g., in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences) or under certain environmental conditions (e.g., inducible regulatory sequences). It will be appreciated by those skilled in the art that the design of the expression vector can depend on such factors as the choice of the host cell to be transformed, the level of expression of polypeptide desired, etc. Typical regulatory sequences useful as promoters of recombinant polypeptide expression (e.g., a heterodimeric trivalent/tetravalent multispecific antibody), include, e.g., but are not limited to, promoters of 3-phosphoglycerate kinase and other glycolytic enzymes. Inducible yeast promoters include, among others, promoters from alcohol dehydrogenase, isocytochrome C, and enzymes responsible for maltose and galactose utilization. In one embodiment, a polynucleotide encoding a heterodimeric trivalent/tetravalent multispecific antibody of the present technology is operably-linked to an ara B promoter and expressible in a host cell. See U.S. Pat. No. 5,028,530. The expression vectors of the present technology can be introduced into host cells to thereby produce polypeptides or peptides, including fusion polypeptides, encoded by nucleic acids as described herein (e.g., heterodimeric trivalent/tetravalent multispecific antibody, etc.).

Another aspect of the present technology pertains to heterodimeric trivalent/tetravalent multispecific antibody-expressing host cells, which contain a nucleic acid encoding one or more heterodimeric trivalent/tetravalent multispecific antibodies. A variety of host-expression vector systems may be utilized to express the heterodimeric trivalent/tetravalent multispecific antibodies of the present disclosure. Such host-expression systems represent vehicles by which the coding sequences of the heterodimeric trivalent/tetravalent multispecific antibodies of the present disclosure may be produced and subsequently purified, but also represent cells which may, when transformed or transfected with the appropriate nucleotide coding sequences, express the molecules of the present disclosure in situ. These include, but are not limited to, microorganisms such as bacteria (e.g., E. coli and B. subtilis) transformed with recombinant bacteriophage DNA, plasmid DNA or cosmid DNA, expression vectors containing coding sequences for the heterodimeric trivalent/tetravalent multispecific antibodies of the present disclosure; yeast (e.g., Saccharomyces Pichia) transformed with recombinant yeast expression vectors containing sequences encoding the heterodimeric trivalent/tetravalent multispecific antibodies of the present disclosure; insect cell systems infected with recombinant virus expression vectors (e.g., baculovirus) containing the sequences encoding the heterodimeric trivalent/tetravalent multispecific antibodies of the present disclosure; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing sequences encoding the heterodimeric trivalent/tetravalent multispecific antibodies of the present disclosure; or mammalian cell systems (e.g., COS, CHO, BHK, 293, 293T, 3T3 cells, lymphotic cells (see U.S. Pat. No. 5,807,715), Per C.6 cells (human retinal cells developed by Crucell) harboring recombinant expression constructs containing promoters derived from the genome of mammalian cells (e.g., metallothionein promoter) or from mammalian viruses (e.g., the adenovirus late promoter; the vaccinia virus 7.5K promoter).

The recombinant expression vectors of the present technology can be designed for expression of a heterodimeric trivalent/tetravalent multispecific antibody in prokaryotic or eukaryotic cells. For example, a heterodimeric trivalent/tetravalent multispecific antibody can be expressed in bacterial cells such as Escherichia coli, insect cells (using baculovirus expression vectors), fungal cells, e.g., yeast, yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, e.g., using T7 promoter regulatory sequences and T7 polymerase. Methods useful for the preparation and screening of polypeptides having a predetermined property, e.g., heterodimeric trivalent/tetravalent multispecific antibody, via expression of stochastically generated polynucleotide sequences have been previously described. See U.S. Pat. Nos. 5,763,192; 5,723,323; 5,814,476; 5,817,483; 5,824,514; 5,976,862; 6,492,107; 6,569,641.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression of recombinant polypeptide; (ii) to increase the solubility of the recombinant polypeptide; and (iii) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding polypeptide, or polypeptide A, respectively, to the target recombinant polypeptide.

Examples of suitable inducible non-fusion E. coli expression vectors include pTrc (Amrann et al., (1988) Gene 69: 301-315) and pET 11d (Studier et al., GENE EXPRESSION TECHNOLOGY: METHODS IN ENZYMOLOGY 185, Academic Press, San Diego, Calif. (1990) 60-89). Methods for targeted assembly of distinct active peptide or protein domains to yield multifunctional polypeptides via polypeptide fusion have been described by Pack et al., U.S. Pat. Nos. 6,294,353; 6,692,935. One strategy to maximize recombinant polypeptide expression, e.g., a heterodimeric trivalent/tetravalent multispecific antibody, in E. coli is to express the polypeptide in host bacteria with an impaired capacity to proteolytically cleave the recombinant polypeptide. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in the expression host, e.g., E. coli (See, e.g., Wada, et al., 1992. Nucl. Acids Res. 20: 2111-2118). Such alteration of nucleic acid sequences of the present technology can be carried out by standard DNA synthesis techniques.

In another embodiment, the heterodimeric trivalent/tetravalent multispecific antibody expression vector is a yeast expression vector. Examples of vectors for expression in yeast Saccharomyces cerevisiae include pYepSec1 (Baldari, et al., 1987. EMBO J. 6: 229-234), pMFa (Kurjan and Herskowitz, Cell 30: 933-943, 1982), pJRY88 (Schultz et al., Gene 54: 113-123, 1987), pYES2 (Invitrogen Corporation, San Diego, Calif.), and picZ (Invitrogen Corp, San Diego, Calif.). Alternatively, a heterodimeric trivalent/tetravalent multispecific antibody can be expressed in insect cells using baculovirus expression vectors. Baculovirus vectors available for expression of polypeptides, e.g., heterodimeric trivalent/tetravalent multispecific antibody, in cultured insect cells (e.g., SF9 cells) include the pAc series (Smith, et al., Mol. Cell. Biol. 3: 2156-2165, 1983) and the pVL series (Lucklow and Summers, 1989. Virology 170: 31-39).

In yet another embodiment, a nucleic acid encoding a heterodimeric trivalent/tetravalent multispecific antibody of the present technology is expressed in mammalian cells using a mammalian expression vector. Examples of mammalian expression vectors include, e.g., but are not limited to, pCDM8 (Seed, Nature 329: 840, 1987) and pMT2PC (Kaufman, et al., EMBO J. 6: 187-195, 1987). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells that are useful for expression of the heterodimeric trivalent/tetravalent multispecific antibody of the present technology, see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid in a particular cell type (e.g., tissue-specific regulatory elements). Tissue-specific regulatory elements are known in the art. Non-limiting examples of suitable tissue-specific promoters include the albumin promoter (liver-specific; Pinkert, et al., Genes Dev. 1: 268-277, 1987), lymphoid-specific promoters (Calame and Eaton, Adv. Immunol. 43: 235-275, 1988), promoters of T cell receptors (Winoto and Baltimore, EMBO J. 8: 729-733, 1989) and immunoglobulins (Banerji, et al., 1983. Cell 33: 729-740; Queen and Baltimore, Cell 33: 741-748, 1983.), neuron-specific promoters (e.g., the neurofilament promoter; Byrne and Ruddle, Proc. Natl. Acad. Sci. USA 86: 5473-5477, 1989), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, Science 249: 374-379, 1990) and the α-fetoprotein promoter (Campes and Tilghman, Genes Dev. 3: 537-546, 1989).

Another aspect of the present methods pertains to host cells into which a recombinant expression vector of the present technology has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a heterodimeric trivalent/tetravalent multispecific antibody can be expressed in bacterial cells such as E. coli, insect cells, yeast or mammalian cells. Mammalian cells are a suitable host for expressing nucleotide segments encoding immunoglobulins or fragments thereof. See Winnacker, From Genes To Clones, (VCH Publishers, N Y, 1987). A number of suitable host cell lines capable of secreting intact heterologous proteins have been developed in the art, and include Chinese hamster ovary (CHO) cell lines, various COS cell lines, HeLa cells, L cells and myeloma cell lines. In some embodiments, the cells are non-human. For example, mammalian cells such as Chinese hamster ovary cells (CHO), in conjunction with a vector such as the major intermediate early gene promoter element from human cytomegalovirus can be an effective expression system for immunoglobulins (Foecking et al., 1998, Gene 45:101; Cockett et al., 1990, BioTechnology 8:2).

Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Queen et al., Immunol. Rev. 89: 49, 1986. Illustrative expression control sequences are promoters derived from endogenous genes, cytomegalovirus, SV40, adenovirus, bovine papillomavirus, and the like. Co et al., J Immunol. 148: 1149, 1992. Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, biolistics or viral-based transfection. Other methods used to transform mammalian cells include the use of polybrene, protoplast fusion, liposomes, electroporation, and microinjection (See generally, Sambrook et al., Molecular Cloning). Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals. The vectors containing the DNA segments of interest can be transferred into the host cell by well-known methods, depending on the type of cellular host.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the heterodimeric trivalent/tetravalent multispecific antibody or can be introduced on a separate vector. Cells stably transfected with the introduced nucleic acid can be identified by drug selection (e.g., cells that have incorporated the selectable marker gene will survive, while the other cells die).

A host cell that includes a heterodimeric trivalent/tetravalent multispecific antibody of the present technology, such as a prokaryotic or eukaryotic host cell in culture, can be used to produce (i.e., express) a recombinant heterodimeric trivalent/tetravalent multispecific antibody. In one embodiment, the method comprises culturing the host cell (into which a recombinant expression vector encoding the heterodimeric trivalent/tetravalent multispecific antibody has been introduced) in a suitable medium such that the heterodimeric trivalent/tetravalent multispecific antibody is produced. In another embodiment, the method further comprises the step of isolating the heterodimeric trivalent/tetravalent multispecific antibody from the medium or the host cell. Once expressed, collections of the heterodimeric trivalent/tetravalent multispecific antibody, e.g., the heterodimeric trivalent/tetravalent multispecific antibodies or the heterodimeric trivalent/tetravalent multispecific antibody-related polypeptides are purified from culture media and host cells. The heterodimeric trivalent/tetravalent multispecific antibody can be purified according to standard procedures of the art, including HPLC purification, column chromatography, gel electrophoresis and the like. In one embodiment, the heterodimeric trivalent/tetravalent multispecific antibody is produced in a host organism by the method of Boss et al., U.S. Pat. No. 4,816,397. Usually, heterodimeric trivalent/tetravalent multispecific antibody chains are expressed with signal sequences and are thus released to the culture media. However, if the heterodimeric trivalent/tetravalent multispecific antibody chains are not naturally secreted by host cells, the heterodimeric trivalent/tetravalent multispecific antibody chains can be released by treatment with mild detergent. Purification of recombinant polypeptides is well known in the art and includes ammonium sulfate precipitation, affinity chromatography purification technique, column chromatography, ion exchange purification technique, gel electrophoresis and the like (See generally Scopes, Protein Purification (Springer-Verlag, N.Y., 1982).

Polynucleotides encoding heterodimeric trivalent/tetravalent multispecific antibodies, e.g., the heterodimeric trivalent/tetravalent multispecific antibody coding sequences, can be incorporated in transgenes for introduction into the genome of a transgenic animal and subsequent expression in the milk of the transgenic animal. See, e.g., U.S. Pat. Nos. 5,741,957, 5,304,489, and 5,849,992. Suitable transgenes include coding sequences for light and/or heavy chains in operable linkage with a promoter and enhancer from a mammary gland specific gene, such as casein or β-lactoglobulin. For production of transgenic animals, transgenes can be microinjected into fertilized oocytes, or can be incorporated into the genome of embryonic stem cells, and the nuclei of such cells transferred into enucleated oocytes.

In bacterial systems, a number of expression vectors may be advantageously selected depending upon the use intended for the molecule being expressed. For example, when a large quantity of such a protein is to be produced, for the generation of pharmaceutical compositions of an antibody, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. For example, in certain embodiments, the polypeptides of a heterodimeric trivalent/tetravalent multispecific antibody of the present disclosure may be expressed as a single gene product (e.g., as a single polypeptide chain, i.e., as a polyprotein precursor), requiring proteolytic cleavage by native or recombinant cellular mechanisms to form the separate polypeptides of the heterodimeric trivalent/tetravalent multispecific antibodies of the present disclosure. The present disclosure thus encompasses engineering a nucleic acid sequence to encode a polyprotein precursor molecule comprising the polypeptides of the heterodimeric trivalent/tetravalent multispecific antibodies of the present disclosure, which includes coding sequences capable of directing post translational cleavage of said polyprotein precursor. Post-translational cleavage of the polyprotein precursor results in the polypeptides of the heterodimeric trivalent/tetravalent multispecific antibodies of the present disclosure.

The post translational cleavage of the precursor molecule comprising the polypeptides of a heterodimeric trivalent/tetravalent multispecific antibody of the present disclosure may occur in vivo (i.e., within the host cell by native or recombinant cell systems/mechanisms, e.g. furin cleavage at an appropriate site) or may occur in vitro (e.g., incubation of said polypeptide chain in a composition comprising proteases or peptidases of known activity and/or in a composition comprising conditions or reagents known to foster the desired proteolytic action). Purification and modification of recombinant proteins are well known in the art such that the design of the polyprotein precursor could include a number of embodiments readily appreciated by a skilled artisan. Any known proteases or peptidases known in the art can be used for the described modification of the precursor molecule, e.g., thrombin (which recognizes the amino acid sequence LVPR\hat{ }GS (SEQ ID NO: 2500)), or factor Xa (which recognizes the amino acid sequence I(E/D)GR\hat{ } (SEQ ID NO: 2501) (Nagani et al., 1985, PNAS USA 82:7252-7255, and reviewed in Jenny et al., 2003, Protein Expr. Purif. 31:1-11, each of which is incorporated by reference herein in its entirety)), enterokinase (which recognizes the amino acid sequence DDDDK\hat{ } (SEQ ID NO: 2502) (Collins-Racie et al., 1995, Biotechnol. 13:982-987 hereby incorporated by reference herein in its entirety)), furin (which recognizes the amino acid sequence RXXR\hat{ }, with a preference for RX(K/R)R\hat{ } (SEQ ID NO: 2503 and SEQ ID NO: 2504, respectively) (additional R at P6 position appears to enhance cleavage)), and AcTEV (which recognizes the amino acid sequence ENLYFQ\hat{ }G (SEQ ID NO: 2505) (Parks et al., 1994, Anal. Biochem. 216:413 hereby incorporated by reference herein in its entirety)) and the Foot and Mouth Disease Virus Protease C3.

Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 293T, 3T3, WI38, BT483, Hs578T, HTB2, BT20 and T47D, CRL7030 and Hs578Bst.

For long-term, high-yield production of recombinant proteins, stable expression is desirable. For example, cell lines which stably express an antibody of the present disclosure may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibodies of the present disclosure. Such engineered cell lines may be particularly useful in screening and evaluation of compounds that interact directly or indirectly with the heterodimeric trivalent/tetravalent multi specific antibodies of the present disclosure.

A number of selection systems may be used, including but not limited to the herpes simplex virus thymidine kinase (Wigler et al., 1977, Cell 11: 223), hypoxanthine-guanine phosphoribosyltransferase (Szybalska & Szybalski, 1992, Proc. Natl. Acad. Sci. USA 48: 202), and adenine phosphoribosyltransferase (Lowy et al., 1980, Cell 22: 817) genes can be employed in tk-, hgprt- or aprt-cells, respectively. Also, antimetabolite resistance can be used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78: 1527); gpt, which confers resistance to mycophenolic acid (Mulligan & Berg, 1981, Proc. Natl. Acad. Sci. USA 78: 2072); neo, which confers resistance to the aminoglycoside G-418 Clinical Pharmacy 12: 488-505; Wu and Wu, 1991, 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1; and hygro, which confers resistance to hygromycin (Santerre et al., 1984, Gene 30:147).

The expression levels of a heterodimeric trivalent/tetravalent multispecific antibody of the present disclosure can be increased by vector amplification (for a review, see Bebbington and Hentschel, The use of vectors based on gene amplification for the expression of cloned genes in mammalian cells in DNA cloning, Vol. 3 (Academic Press, New York, 1987). When a marker in the vector system expressing an antibody is amplifiable, increase in the level of inhibitor present in culture of host cell will increase the number of copies of the selection marker gene. Since the amplified region is associated with the nucleotide sequence of a polypeptide of the heterodimeric trivalent/tetravalent multispecific antibody molecule, production of the polypeptide will also increase (Crouse et al., 1983, Mol. Cell. Biol. 3:257).

The host cell may be co-transfected with a plurality of expression vectors of the present disclosure, wherein each expression vector encodes at least one and no more than three of the first, second, third, or fourth polypeptide chains of the heterodimeric trivalent/tetravalent multispecific antibody. Alternatively, a single vector may be used which encodes the first, second, third, and fourth polypeptide chains of the heterodimeric trivalent/tetravalent multispecific antibody. The coding sequences for the polypeptides of the heterodimeric trivalent/tetravalent multispecific antibodies of the present disclosure may comprise cDNA or genomic DNA.

Once a molecule of the present disclosure (i.e., heterodimeric trivalent/tetravalent multispecific antibodies) has been recombinantly expressed, it may be purified by any method known in the art for purification of polypeptides, polyproteins or heterodimeric trivalent/tetravalent multispecific antibodies (e.g., analogous to antibody purification schemes based on antigen selectivity) for example, by chromatography (e.g., ion exchange, affinity, particularly by affinity for the specific antigen (optionally after Protein A selection where the heterodimeric trivalent/tetravalent multispecific antibodies molecule comprises an Fc domain (or portion thereof)), and sizing column chromatography), centrifugation, differential solubility, or by any other standard technique for the purification of polypeptides, polyproteins or heterodimeric trivalent/tetravalent multispecific antibodies.

Labeled Heterodimeric trivalent/tetravalent multispecific antibodies. In one embodiment, the heterodimeric trivalent/tetravalent multispecific antibody of the present technology is coupled with a label moiety, i.e., detectable group. The particular label or detectable group conjugated to the heterodimeric trivalent/tetravalent multispecific antibody is not a critical aspect of the technology, so long as it does not significantly interfere with the specific binding of the heterodimeric trivalent/tetravalent multispecific antibody of the present technology to its target antigens. The detectable group can be any material having a detectable physical or chemical property. Such detectable labels have been well-developed in the field of immunoassays and imaging. In general, almost any label useful in such methods can be applied to the present technology. Thus, a label is any composition detectable by spectroscopic, photochemical, biochemical, immunochemical, electrical, optical or chemical means. Labels useful in the practice of the present technology include magnetic beads (e.g., Dynabeads™), fluorescent dyes (e.g., fluorescein isothiocyanate, Texas red, rhodamine, and the like), radiolabels (e.g., ³H, ¹⁴C, ³⁵S ¹²⁵I, ¹²¹I, ¹³¹I, ¹¹²In, ⁹⁹mTc), other imaging agents such as microbubbles (for ultrasound imaging), ¹⁸F, 1^{1C}, ¹⁵O, (for Positron emission tomography), ^{99m}Tc, ¹¹¹In (for Single photon emission tomography), enzymes (e.g., horse radish peroxidase, alkaline phosphatase and others commonly used in an ELISA), and calorimetric labels such as colloidal gold or colored glass or plastic (e.g., polystyrene, polypropylene, latex, and the like) beads. Patents that describe the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes. See also Handbook of Fluorescent Probes and Research Chemicals (6^{th} Ed., Molecular Probes, Inc., Eugene Oreg.).

The label can be coupled directly or indirectly to the desired component of an assay according to methods well known in the art. As indicated above, a wide variety of labels can be used, with the choice of label depending on factors such as required sensitivity, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, e.g., biotin, thyroxine, and cortisol, it can be used in conjunction with the labeled, naturally-occurring anti-ligands. Alternatively, any haptenic or antigenic compound can be used in combination with an antibody, e.g., a heterodimeric trivalent/tetravalent multispecific antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds useful as labeling moieties, include, but are not limited to, e.g., fluorescein and its derivatives, rhodamine and its derivatives, dansyl, umbelliferone, and the like. Chemiluminescent compounds useful as labeling moieties, include, but are not limited to, e.g., luciferin, and 2,3-dihydrophthalazinediones, e.g., luminol. For a review of various labeling or signal-producing systems which can be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Finally simple colorimetric labels can be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies, e.g., the heterodimeric trivalent/tetravalent multispecific antibodies. In this case, antigen-coated particles are agglutinated by samples comprising the target antibodies. In this format, none of the components need be labeled and the presence of the target antibody is detected by simple visual inspection.

Fusion Proteins. In one embodiment, the heterodimeric trivalent/tetravalent multispecific antibody of the present technology is a fusion protein. In some embodiments, the heterodimeric trivalent/tetravalent multispecific antibodies of the present technology, when fused to a second protein, can be used as an antigenic tag. Examples of domains that can be fused to polypeptides include not only heterologous signal sequences, but also other heterologous functional regions. The fusion does not necessarily need to be direct, but can occur through linker sequences. Moreover, fusion proteins of the present technology can also be engineered to improve characteristics of the heterodimeric trivalent/tetravalent multispecific antibodies. For instance, a region of additional amino acids, particularly charged amino acids, can be added to the N-terminus of the heterodimeric trivalent/tetravalent multispecific antibody to improve stability and persistence during purification from the host cell or subsequent handling and storage. Also, peptide moieties can be added to a heterodimeric trivalent/tetravalent multispecific antibody to facilitate purification. Such regions can be removed prior to final preparation of the heterodimeric trivalent/tetravalent multispecific antibody. The addition of peptide moieties to facilitate handling of polypeptides may be accomplished using familiar and routine techniques in the art. The heterodimeric trivalent/tetravalent multispecific antibody of the present technology can be fused to marker sequences, such as a peptide which facilitates purification of the fused polypeptide. In select embodiments, the marker amino acid sequence is a hexa-histidine peptide (SEQ ID NO: 2510), such as the tag provided in a pQE vector (QIAGEN, Inc., Chatsworth, Calif.), among others, many of which are commercially available. As described in Gentz et al., Proc. Natl. Acad. Sci. USA 86: 821-824, 1989, for instance, hexa-histidine (SEQ ID NO: 2510) provides for convenient purification of the fusion protein. Another peptide tag useful for purification, the “HA” tag, corresponds to an epitope derived from the influenza hemagglutinin protein. Wilson et al., Cell 37: 767, 1984.

Thus, any of these above fusion proteins can be engineered using the polynucleotides or the polypeptides of the present technology. Also, in some embodiments, the fusion proteins described herein show an increased half-life in vivo.

Fusion proteins having disulfide-linked dimeric structures (due to the IgG) can be more efficient in binding and neutralizing other molecules compared to the monomeric secreted protein or protein fragment alone. Fountoulakis et al., J. Biochem. 270: 3958-3964, 1995.

Similarly, EP-A-O 464 533 (Canadian counterpart 2045869) discloses fusion proteins comprising various portions of constant region of immunoglobulin molecules together with another human protein or a fragment thereof. In many cases, the Fc part in a fusion protein is beneficial in therapy and diagnosis, and thus can result in, e.g., improved pharmacokinetic properties. See EP-A 0232 262. Alternatively, deleting or modifying the Fc part after the fusion protein has been expressed, detected, and purified, may be desired. For example, the Fc portion can hinder therapy and diagnosis if the fusion protein is used as an antigen for immunizations. In drug discovery, e.g., human proteins, such as hIL-5, have been fused with Fc portions for the purpose of high-throughput screening assays to identify antagonists of hIL-5. Bennett et al., J. Molecular Recognition 8: 52-58, 1995; Johanson et al., J. Biol. Chem., 270: 9459-9471, 1995.

In some embodiments, the heterodimeric trivalent/tetravalent multispecific antibody of the present technology may be conjugated to a therapeutic agent or a payload. Examples of a payload include a toxin, a protein such as tumor necrosis factor, interferons including, but not limited to, α-interferon (IFN-α), β-interferon (IFN-β), nerve growth factor (NGF), platelet derived growth factor (PDGF), tissue plasminogen activator (TPA), an apoptotic agent (e.g., TNF-α, TNF-β, AIM I as disclosed in PCT Publication No. WO 97/33899), AIM II (see, PCT Publication No. WO 97/34911), Fas ligand (Takahashi et al., J. Immunol., 6:1567-1574, 1994), and VEGI (PCT Publication No. WO 99/23105), a thrombotic agent or an anti-angiogenic agent (e.g., angiostatin or endostatin), or a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”), macrophage colony stimulating factor, (“M-CSF”), or a growth factor (e.g., growth hormone (“GH”); proteases, or ribonucleases. Examples of therapeutic agents include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Other examples of therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC), and anti-mitotic agents (e.g., vincristine and vinblastine).

### B. Identifying and Characterizing the Heterodimeric Trivalent/Tetravalent Multispecific Antibodies of the Present Technology

Methods for identifying and/or screening the heterodimeric trivalent/tetravalent multispecific antibodies of the present technology. Methods useful to identify and screen antibodies that possess the desired specificity to a target antigen include any immunologically-mediated techniques known within the art. Components of an immune response can be detected in vitro by various methods that are well known to those of ordinary skill in the art. For example, (1) cytotoxic T lymphocytes can be incubated with radioactively labeled target cells and the lysis of these target cells detected by the release of radioactivity; (2) helper T lymphocytes can be incubated with antigens and antigen presenting cells and the synthesis and secretion of cytokines measured by standard methods (Windhagen A et al., Immunity, 2: 373-80, 1995); (3) antigen presenting cells can be incubated with whole protein antigen and the presentation of that antigen on MHC detected by either T lymphocyte activation assays or biophysical methods (Harding et al., Proc. Natl. Acad. Sci., 86: 4230-4, 1989); (4) mast cells can be incubated with reagents that cross-link their Fc-epsilon receptors and histamine release measured by enzyme immunoassay (Siraganian et al., TIPS, 4: 432-437, 1983); and (5) enzyme-linked immunosorbent assay (ELISA).

Similarly, products of an immune response in either a model organism (e.g., mouse) or a human subject can also be detected by various methods that are well known to those of ordinary skill in the art. For example, (1) the production of antibodies in response to vaccination can be readily detected by standard methods currently used in clinical laboratories, e.g., an ELISA; (2) the migration of immune cells to sites of inflammation can be detected by scratching the surface of skin and placing a sterile container to capture the migrating cells over scratch site (Peters et al., Blood, 72: 1310-5, 1988); (3) the proliferation of peripheral blood mononuclear cells (PBMCs) in response to mitogens or mixed lymphocyte reaction can be measured using ³H-thymidine; (4) the phagocytic capacity of granulocytes, macrophages, and other phagocytes in PBMCs can be measured by placing PBMCs in wells together with labeled particles (Peters et al., Blood, 72: 1310-5, 1988); and (5) the differentiation of immune system cells can be measured by labeling PBMCs with antibodies to CD molecules such as CD4 and CD8 and measuring the fraction of the PBMCs expressing these markers.

In one embodiment, heterodimeric trivalent/tetravalent multispecific antibodies of the present technology are selected using display of target antigen peptides on the surface of replicable genetic packages. See, e.g., U.S. Pat. Nos. 5,514,548; 5,837,500; 5,871,907; 5,885,793; 5,969,108; 6,225,447; 6,291,650; 6,492,160; EP 585 287; EP 605522; EP 616640; EP 1024191; EP 589 877; EP 774 511; EP 844 306. Methods useful for producing/selecting a filamentous bacteriophage particle containing a phagemid genome encoding for a binding molecule with a desired specificity has been described. See, e.g., EP 774 511; U.S. Pat. Nos. 5,871,907; 5,969,108; 6,225,447; 6,291,650; 6,492,160.

In some embodiments, heterodimeric trivalent/tetravalent multispecific antibodies of the present technology are selected using display of target antigen peptides on the surface of a yeast host cell. Methods useful for the isolation of scFv polypeptides by yeast surface display have been described by Kieke et al., Protein Eng. 1997 November; 10(11): 1303-10.

In some embodiments, heterodimeric trivalent/tetravalent multispecific antibodies of the present technology are selected using ribosome display. Methods useful for identifying ligands in peptide libraries using ribosome display have been described by Mattheakis et al., Proc. Natl. Acad. Sci. USA 91: 9022-26, 1994; and Hanes et al., Proc. Natl. Acad. Sci. USA 94: 4937-42, 1997.

In certain embodiments, heterodimeric trivalent/tetravalent multispecific antibodies of the present technology are selected using tRNA display of target antigen peptides. Methods useful for in vitro selection of ligands using tRNA display have been described by Merryman et al., Chem. Biol., 9: 741-46, 2002.

In one embodiment, heterodimeric trivalent/tetravalent multispecific antibodies of the present technology are selected using RNA display. Methods useful for selecting peptides and proteins using RNA display libraries have been described by Roberts et al. Proc. Natl. Acad. Sci. USA, 94: 12297-302, 1997; and Nemoto et al., FEBS Lett., 414: 405-8, 1997. Methods useful for selecting peptides and proteins using unnatural RNA display libraries have been described by Frankel et al., Curr. Opin. Struct. Biol., 13: 506-12, 2003.

In some embodiments, heterodimeric trivalent/tetravalent multispecific antibodies of the present technology are expressed in the periplasm of gram negative bacteria and mixed with labeled target antigen. See WO 02/34886. In clones expressing recombinant polypeptides with affinity for a target antigen, the concentration of the labeled target antigen bound to the heterodimeric trivalent/tetravalent multispecific antibodies is increased and allows the cells to be isolated from the rest of the library as described in Harvey et al., Proc. Natl. Acad. Sci. 22: 9193-98 2004 and U.S. Pat. Publication No. 2004/0058403.

After selection of the desired heterodimeric trivalent/tetravalent multispecific antibodies, it is contemplated that said antibodies can be produced in large volume by any technique known to those skilled in the art, e.g., prokaryotic or eukaryotic cell expression and the like. For example, the heterodimeric trivalent/tetravalent multispecific antibodies can be produced by using conventional techniques to construct an expression vector that encodes an antibody heavy chain and/or light chain in which the CDRs and, if necessary, a minimal portion of the variable region framework, that are required to retain original species antibody binding specificity (as engineered according to the techniques described herein) are derived from the originating species antibody and the remainder of the antibody is derived from a target species immunoglobulin which can be manipulated as described herein, thereby producing a vector for the expression of a hybrid antibody heavy chain.

Measurement of Antigen Binding. In some embodiments, an antigen binding assay refers to an assay format wherein a target antigen and a heterodimeric trivalent/tetravalent multispecific antibody are mixed under conditions suitable for binding between the target antigen and the heterodimeric trivalent/tetravalent multispecific antibody and assessing the amount of binding between the target antigen and the heterodimeric trivalent/tetravalent multispecific antibody. The amount of binding is compared with a suitable control, which can be the amount of binding in the absence of the target antigen, the amount of the binding in the presence of a non-specific immunoglobulin composition, or both. The amount of binding can be assessed by any suitable method. Binding assay methods include, e.g., ELISA, radioimmunoassays, scintillation proximity assays, fluorescence energy transfer assays, liquid chromatography, membrane filtration assays, and the like. Biophysical assays for the direct measurement of target antigen binding to a heterodimeric trivalent/tetravalent multispecific antibody are, e.g., nuclear magnetic resonance, fluorescence, fluorescence polarization, surface plasmon resonance (BIACORE chips) and the like. Specific binding is determined by standard assays known in the art, e.g., radioligand binding assays, ELISA, FRET, immunoprecipitation, SPR, NMR (2D-NMR), mass spectroscopy and the like. If the specific binding of a candidate heterodimeric trivalent/tetravalent multispecific antibody is at least 1 percent greater than the binding observed in the absence of the candidate heterodimeric trivalent/tetravalent multispecific antibody, the candidate heterodimeric trivalent/tetravalent multi specific antibody is useful as a heterodimeric trivalent/tetravalent multispecific antibody of the present technology.

Measurement of Target Antigen Neutralization. As used here, “target antigen neutralization” refers to reduction of the activity and/or expression of a target antigen through the binding of a heterodimeric trivalent/tetravalent multispecific antibody disclosed herein. The capacity of heterodimeric trivalent/tetravalent multispecific antibodies of the present technology to neutralize activity/expression of a target antigen may be assessed in vitro or in vivo using methods known in the art.

### Uses of the Heterodimeric Trivalent/Tetravalent Multispecific Antibodies of the Present Technology

General. The heterodimeric trivalent/tetravalent multispecific antibodies of the present technology are useful in methods known in the art relating to the localization and/or quantitation of a target antigen (e.g., for use in measuring levels of the target antigen within appropriate physiological samples, for use in diagnostic methods, for use in imaging the target antigen, and the like). Antibodies of the present technology are useful to isolate a target antigen by standard techniques, such as affinity chromatography or immunoprecipitation. A heterodimeric trivalent/tetravalent multispecific antibody of the present technology can facilitate the purification of natural immunoreactive target antigens from biological samples, e.g., mammalian sera or cells as well as recombinantly-produced immunoreactive target antigens expressed in a host system. Moreover, heterodimeric trivalent/tetravalent multispecific antibodies can be used to detect an immunoreactive target antigen (e.g., in plasma, a cellular lysate or cell supernatant) in order to evaluate the abundance and pattern of expression of the immunoreactive molecule. The heterodimeric trivalent/tetravalent multispecific antibodies of the present technology can be used diagnostically to monitor immunoreactive target antigen levels in tissue as part of a clinical testing procedure, e.g., to determine the efficacy of a given treatment regimen. As noted above, the detection can be facilitated by coupling (i.e., physically linking) the heterodimeric trivalent/tetravalent multispecific antibodies of the present technology to a detectable sub stance.

Detection of target antigen. An exemplary method for detecting the presence or absence of an immunoreactive target antigen in a biological sample involves obtaining a biological sample from a test subject and contacting the biological sample with a heterodimeric trivalent/tetravalent multispecific antibody of the present technology capable of detecting an immunoreactive target antigen such that the presence of an immunoreactive target antigen is detected in the biological sample. Detection may be accomplished by means of a detectable label attached to the antibody.

The term “labeled” with regard to the heterodimeric trivalent/tetravalent multispecific antibody is intended to encompass direct labeling of the antibody by coupling (i.e., physically linking) a detectable substance to the antibody, as well as indirect labeling of the antibody by reactivity with another compound that is directly labeled, such as a secondary antibody. Examples of indirect labeling include detection of a primary antibody using a fluorescently-labeled secondary antibody and end-labeling of a DNA probe with biotin such that it can be detected with fluorescently-labeled streptavidin.

In some embodiments, the heterodimeric trivalent/tetravalent multispecific antibodies disclosed herein are conjugated to one or more detectable labels. For such uses, heterodimeric trivalent/tetravalent multispecific antibodies may be detectably labeled by covalent or non-covalent attachment of a chromogenic, enzymatic, radioisotopic, isotopic, fluorescent, toxic, chemiluminescent, nuclear magnetic resonance contrast agent or other label.

Examples of suitable chromogenic labels include diaminobenzidine and 4-hydroxyazo-benzene-2-carboxylic acid. Examples of suitable enzyme labels include malate dehydrogenase, staphylococcal nuclease, Δ-5-steroid isomerase, yeast-alcohol dehydrogenase, α-glycerol phosphate dehydrogenase, triose phosphate isomerase, peroxidase, alkaline phosphatase, asparaginase, glucose oxidase, β-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. ¹¹¹In is an exemplary isotope where in vivo imaging is used since it avoids the problem of dehalogenation of the ¹²⁵I or ¹³¹I-labeled heterodimeric trivalent/tetravalent multispecific antibodies by the liver. In addition, this isotope has a more favorable gamma emission energy for imaging (Perkins et al, Eur. J. Nucl. Med. 70:296-301 (1985); Carasquillo et al., J. Nucl. Med. 25:281-287 (1987)). For example, ¹¹¹In coupled to monoclonal antibodies with 1-(P-isothiocyanatobenzyl)-DPTA exhibits little uptake in non-tumorous tissues, particularly the liver, and enhances specificity of tumor localization (Esteban et al., J. Nucl. Med. 28:861-870 (1987)). Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, a Green Fluorescent Protein (GFP) label, an o-phthaldehyde label, and a fluorescamine label. Examples of suitable toxin labels include diphtheria toxin, ricin, and cholera toxin.

Examples of chemiluminescent labels include a luminol label, an isoluminol label, an aromatic acridinium ester label, an imidazole label, an acridinium salt label, an oxalate ester label, a luciferin label, a luciferase label, and an aequorin label. Examples of nuclear magnetic resonance contrasting agents include heavy metal nuclei such as Gd, Mn, and iron.

The detection method of the present technology can be used to detect an immunoreactive target antigen in a biological sample in vitro as well as in vivo. In vitro techniques for detection of an immunoreactive target antigen include enzyme linked immunosorbent assays (ELISAs), Western blots, immunoprecipitations, radioimmunoassay, and immunofluorescence. Furthermore, in vivo techniques for detection of an immunoreactive target antigen include introducing into a subject a labeled heterodimeric trivalent/tetravalent multispecific antibody. For example, the heterodimeric trivalent/tetravalent multispecific antibody can be labeled with a radioactive marker whose presence and location in a subject can be detected by standard imaging techniques. In one embodiment, the biological sample contains target antigen molecules from the test subject.

Immunoassay and Imaging. A heterodimeric trivalent/tetravalent multispecific antibody of the present technology can be used to assay immunoreactive target antigen levels in a biological sample (e.g., human plasma) using antibody-based techniques. For example, protein expression in tissues can be studied with classical immunohistological methods. Jalkanen, M. et al., J. Cell. Biol. 101: 976-985, 1985; Jalkanen, M. et al., J. Cell. Biol. 105: 3087-3096, 1987. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (¹²⁵I, ¹²¹I, ¹³¹I), and carbon (¹⁴C), sulfur (³⁵S), tritium (³H), indium (¹¹²In), and technetium (⁹⁹mTc), and fluorescent labels, such as fluorescein, rhodamine, and green fluorescent protein (GFP), as well as biotin.

In addition to assaying immunoreactive target antigen levels in a biological sample, heterodimeric trivalent/tetravalent multispecific antibodies of the present technology may be used for in vivo imaging of the target antigen. Antibodies useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the heterodimeric trivalent/tetravalent multispecific antibodies by labeling of nutrients for the relevant scFv clone.

A heterodimeric trivalent/tetravalent multispecific antibody which has been labeled with an appropriate detectable imaging moiety, such as a radioisotope (e.g., ¹³¹I, ¹¹²In, ⁹⁹mTc), a radio-opaque substance, or a material detectable by nuclear magnetic resonance, is introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled heterodimeric trivalent/tetravalent multispecific antibody will then accumulate at the location of cells which contain the specific target antigen. For example, labeled heterodimeric trivalent/tetravalent multispecific antibodies of the present technology will accumulate within the subject in cells and tissues in which the target antigen has localized.

Thus, the present technology provides a diagnostic method of a medical condition, which involves: (a) assaying the expression of immunoreactive target antigen by measuring binding of a heterodimeric trivalent/tetravalent multispecific antibody of the present technology in cells or body fluid of an individual; (b) comparing the amount of immunoreactive target antigen present in the sample with a standard reference, wherein an increase or decrease in immunoreactive target antigen levels compared to the standard is indicative of a medical condition.

Affinity Purification. The heterodimeric trivalent/tetravalent multispecific antibodies of the present technology may be used to purify immunoreactive target antigen from a sample. In some embodiments, the antibodies are immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art (Weir et al., “Handbook of Experimental Immunology” 4th Ed., Blackwell Scientific Publications, Oxford, England, Chapter 10 (1986); Jacoby et al., Meth. Enzym. 34 Academic Press, N.Y. (1974)).

The simplest method to bind the antigen to the antibody-support matrix is to collect the beads in a column and pass the antigen solution down the column. The efficiency of this method depends on the contact time between the immobilized antibody and the antigen, which can be extended by using low flow rates. The immobilized antibody captures the antigen as it flows past. Alternatively, an antigen can be contacted with the antibody-support matrix by mixing the antigen solution with the support (e.g., beads) and rotating or rocking the slurry, allowing maximum contact between the antigen and the immobilized antibody. After the binding reaction has been completed, the slurry is passed into a column for collection of the beads. The beads are washed using a suitable washing buffer and then the pure or substantially pure antigen is eluted.

An antibody or target antigen of interest can be conjugated to a solid support, such as a bead. In addition, a first solid support such as a bead can also be conjugated, if desired, to a second solid support, which can be a second bead or other support, by any suitable means, including those disclosed herein for conjugation of a molecule to a support. Accordingly, any of the conjugation methods and means disclosed herein with reference to conjugation of a molecule to a solid support can also be applied for conjugation of a first support to a second support, where the first and second solid support can be the same or different.

Appropriate linkers, which can be cross-linking agents, for use for conjugating a molecule to a solid support include a variety of agents that can react with a functional group present on a surface of the support, or with the molecule, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. In one exemplary embodiment, a cross-linking agent can be selected to provide a selectively cleavable bond between a target polypeptide and the solid support. For example, a photolabile cross-linker, such as 3-amino-(2-nitrophenyl)propionic acid can be employed as a means for cleaving a target polypeptide from a solid support. (Brown et al., Mol. Divers, pp, 4-12 (1995); Rothschild et al., Nucl. Acids Res., 24:351-66 (1996); and U.S. Pat. No. 5,643,722). Other cross-linking reagents are well-known in the art. (See, e.g., Wong (1991), supra; and Hermanson (1996), supra).

An antibody or target polypeptide can be immobilized on a solid support, such as a bead, through a covalent amide bond formed between a carboxyl group functionalized bead and the amino terminus of the target polypeptide or, conversely, through a covalent amide bond formed between an amino group functionalized bead and the carboxyl terminus of the target polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the target polypeptide is cleaved and can be removed. In such a case, the target polypeptide can be deposited as a beadless patch at the bottom of a well of a solid support or on the flat surface of a solid support. After addition of a matrix solution, the target polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the target polypeptide. Acid lability can also be changed. For example, trityl, monomethoxytrityl, dimethoxytrityl or trimethoxytrityl can be changed to the appropriate p-substituted, or more acid-labile tritylamine derivatives, of the target polypeptide, i.e., trityl ether and tritylamine bonds can be made to the target polypeptide. Accordingly, a target polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

Orthogonally cleavable linkers can also be useful for binding a first solid support, e.g., a bead to a second solid support, or for binding a molecule of interest to a solid support. Using such linkers, a first solid support, e.g., a bead, can be selectively cleaved from a second solid support, without cleaving the target antigen from the support; the target antigen then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a target antigen to the support. As desired, the linkage of the target antigen to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high density binding of the target antigens to the beads, is promoted. Such a linking group can have, e.g., “tree-like” structure, thereby providing a multiplicity of functional groups per attachment site on a solid support. Examples of such linking group; include polylysine, polyglutamic acid, penta-erythrole and tris-hydroxy-aminomethane.

Noncovalent Binding Association. An antibody or target antigen can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a target antigen, e.g., a polypeptide containing an attached trityl group or with a second solid support having hydrophobic character.

A solid support can also be provided with a member of a specific binding pair and, therefore, can be conjugated to a target antigen or a second solid support containing a complementary binding moiety. For example, a bead coated with avidin or with streptavidin can be bound to a target antigen (e.g., a polypeptide) having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a target antigen or a solid support and, conversely, avidin or other biotin binding moiety would be incorporated into the support or the target antigen, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs known to those skilled in the art.

### A. Diagnostic Uses

General. The heterodimeric trivalent/tetravalent multispecific antibodies of the present technology are useful in diagnostic methods. As such, the present technology provides methods using the antibodies in the diagnosis of activity of a molecule of interest in a subject. Heterodimeric trivalent/tetravalent multispecific antibodies of the present technology may be selected such that they have any level of epitope binding specificity and binding affinity to a target antigen. In general, the higher the binding affinity of an antibody, the more stringent wash conditions can be performed in an immunoassay to remove nonspecifically bound material without removing the molecule of interest. Accordingly, heterodimeric trivalent/tetravalent multispecific antibodies of the present technology useful in diagnostic assays usually have binding affinities of about 10⁸M⁻¹, 10⁹M⁻¹, 10¹⁰ M⁻¹, 10¹¹ M⁻¹ or 10¹² M⁻¹. Further, it is desirable that heterodimeric trivalent/tetravalent multispecific antibodies used as diagnostic reagents have a sufficient kinetic on-rate to reach equilibrium under standard conditions in at least 12 h, at least five (5) h, or at least one (1) hour.

Heterodimeric trivalent/tetravalent multispecific antibodies can be used to detect an immunoreactive target antigen in a variety of standard assay formats. Such formats include immunoprecipitation, Western blotting, ELISA, radioimmunoassay, and immunometric assays. See Harlow & Lane, Antibodies, A Laboratory Manual (Cold Spring Harbor Publications, New York, 1988); U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Biological samples can be obtained from any tissue or body fluid of a subject. In certain embodiments, the subject is at an early stage of cancer. In one embodiment, the early stage of cancer is determined by the level or expression pattern of a target antigen in a sample obtained from the subject. In certain embodiments, the sample is selected from the group consisting of urine, blood, serum, plasma, saliva, amniotic fluid, cerebrospinal fluid (CSF), and biopsied body tissue.

Immunometric or sandwich assays are one format for the diagnostic methods of the present technology. See U.S. Pat. Nos. 4,376,110, 4,486,530, 5,914,241, and 5,965,375. Such assays use one antibody, e.g., a heterodimeric trivalent/tetravalent multispecific antibody or a population of heterodimeric trivalent/tetravalent multispecific antibodies immobilized to a solid phase, and another heterodimeric trivalent/tetravalent multispecific antibody or a population of heterodimeric trivalent/tetravalent multispecific antibodies in solution. Typically, the solution heterodimeric trivalent/tetravalent multispecific antibody or population of heterodimeric trivalent/tetravalent multispecific antibodies is labeled. If an antibody population is used, the population can contain antibodies binding to different epitope specificities within the target antigen. Accordingly, the same population can be used for both solid phase and solution antibody. If heterodimeric trivalent/tetravalent multispecific monoclonal antibodies are used, first and second monoclonal heterodimeric trivalent/tetravalent multispecific antibodies having different binding specificities are used for the solid and solution phase. Solid phase (also referred to as “capture”) and solution (also referred to as “detection”) antibodies can be contacted with target antigen in either order or simultaneously. If the solid phase antibody is contacted first, the assay is referred to as being a forward assay. Conversely, if the solution antibody is contacted first, the assay is referred to as being a reverse assay. If the target is contacted with both antibodies simultaneously, the assay is referred to as a simultaneous assay. After contacting the target antigen with the heterodimeric trivalent/tetravalent multispecific antibody, a sample is incubated for a period that usually varies from about 10 min to about 24 hr and is usually about 1 hr. A wash step is then performed to remove components of the sample not specifically bound to the heterodimeric trivalent/tetravalent multispecific antibody being used as a diagnostic reagent. When solid phase and solution antibodies are bound in separate steps, a wash can be performed after either or both binding steps. After washing, binding is quantified, typically by detecting a label linked to the solid phase through binding of labeled solution antibody. Usually for a given pair of antibodies or populations of antibodies and given reaction conditions, a calibration curve is prepared from samples containing known concentrations of target antigen. Concentrations of the immunoreactive target antigen in samples being tested are then read by interpolation from the calibration curve (i.e., standard curve). Analyte can be measured either from the amount of labeled solution antibody bound at equilibrium or by kinetic measurements of bound labeled solution antibody at a series of time points before equilibrium is reached. The slope of such a curve is a measure of the concentration of the target antigen in a sample.

Suitable supports for use in the above methods include, e.g., nitrocellulose membranes, nylon membranes, and derivatized nylon membranes, and also particles, such as agarose, a dextran-based gel, dipsticks, particulates, microspheres, magnetic particles, test tubes, microtiter wells, SEPHADEX™ (Amersham Pharmacia Biotech, Piscataway N.J.), and the like. Immobilization can be by absorption or by covalent attachment. Optionally, heterodimeric trivalent/tetravalent multispecific antibodies can be joined to a linker molecule, such as biotin for attachment to a surface bound linker, such as avidin.

In some embodiments, the present disclosure provides a heterodimeric trivalent/tetravalent multispecific antibody of the present technology conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells containing the antigen. Radioactive levels emitted by the antibody may be detected using positron emission tomography or single photon emission computed tomography.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). U.S. Pat. No. 6,331,175 describes MRI technique and the preparation of antibodies conjugated to a MRI enhancing agent and is incorporated in its entirety by reference. In some embodiments, the diagnostic agents are selected from the group consisting of radioisotopes, enhancing agents for use in magnetic resonance imaging, and fluorescent compounds. In order to load an antibody component with radioactive metals or paramagnetic ions, it may be necessary to react it with a reagent having a long tail to which are attached a multiplicity of chelating groups for binding the ions. Such a tail can be a polymer such as a polylysine, polysaccharide, or other derivatized or derivatizable chain having pendant groups to which can be bound chelating groups such as, e.g., ethylenediaminetetraacetic acid (EDTA), diethylenetriaminepentaacetic acid (DTPA), porphyrins, polyamines, crown ethers, bis-thiosemicarbazones, polyoximes, and like groups known to be useful for this purpose. Chelates may be coupled to the antibodies of the present technology using standard chemistries. The chelate is normally linked to the antibody by a group which enables formation of a bond to the molecule with minimal loss of immunoreactivity and minimal aggregation and/or internal cross-linking. Other methods and reagents for conjugating chelates to antibodies are disclosed in U.S. Pat. No. 4,824,659. Particularly useful metal-chelate combinations include 2-benzyl-DTPA and its monomethyl and cyclohexyl analogs, used with diagnostic isotopes for radio-imaging. The same chelates, when complexed with non-radioactive metals, such as manganese, iron and gadolinium are useful for MM, when used along with the heterodimeric trivalent/tetravalent multispecific antibodies of the present technology.

### B. Therapeutic Uses

The immunoglobulin-related compositions (e.g., heterodimeric trivalent/tetravalent multispecific antibodies) of the present technology are useful for the treatment of a disease or condition. Exemplary diseases or conditions include, but are not limited to cardiovascular disease, diabetes, autoimmune disease, dementia, Parkinson's disease, cancer or Alzheimer's disease. Such treatment can be used in patients identified as having pathological levels of a molecule of interest (e.g., those diagnosed by the methods described herein) or in patients diagnosed with a disease known to be associated with such pathological levels. In one aspect, the present disclosure provides a method for treating cancer in a subject in need thereof, comprising administering to the subject an effective amount of a heterodimeric trivalent/tetravalent multispecific antibody of the present technology. Examples of cancers that can be treated by the antibodies of the present technology include, but are not limited to: lung cancer, colorectal cancer, skin cancer, breast cancer, ovarian cancer, leukemia, pancreatic cancer, and gastric cancer.

The compositions of the present technology may be employed in conjunction with other therapeutic agents useful in the treatment of cancer. For example, the antibodies of the present technology may be separately, sequentially or simultaneously administered with at least one additional therapeutic agent-selected from the group consisting of alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof.

In another aspect, the antibodies of the present technology may be separately, sequentially or simultaneously administered with one or more therapeutic agents useful in the treatment of Alzheimer's disease. Examples of such therapeutic agents include acetylcholine esterase inhibitors such as tacrine (tetrahydroaminoacridine), donepezil hydrochloride, and rivastigmine; gamma-secretase inhibitors; anti-inflammatory agents such as cyclooxygenase II inhibitors; antioxidants such as Vitamin E and ginkolides; immunological approaches, such as, for example, immunization with A beta peptide or administration of anti-A beta peptide antibodies; statins; and direct or indirect neurotropic agents such as Cerebrolysin®, AIT-082 (Emilieu, 2000, Arch. Neurol. 57:454).

The compositions of the present technology may optionally be administered as a single bolus to a subject in need thereof. Alternatively, the dosing regimen may comprise multiple administrations performed at various times after the appearance of tumors or amyloid plaques.

Administration can be carried out by any suitable route, including orally, intranasally, parenterally (intravenously, intramuscularly, intraperitoneally, or subcutaneously), rectally, intracranially, intrathecally, or topically. Administration includes self-administration and the administration by another. It is also to be appreciated that the various modes of treatment of medical conditions as described are intended to mean “substantial”, which includes total but also less than total treatment, and wherein some biologically or medically relevant result is achieved.

In some embodiments, the antibodies of the present technology comprise pharmaceutical formulations which may be administered to subjects in need thereof in one or more doses. Dosage regimens can be adjusted to provide the desired response (e.g., a therapeutic response).

Typically, an effective amount of the antibody compositions of the present technology, sufficient for achieving a therapeutic effect, range from about 0.000001 mg per kilogram body weight per day to about 10,000 mg per kilogram body weight per day. Typically, the dosage ranges are from about 0.0001 mg per kilogram body weight per day to about 100 mg per kilogram body weight per day. For administration of heterodimeric trivalent/tetravalent multispecific antibodies, the dosage ranges from about 0.0001 to 100 mg/kg, and more usually 0.01 to 5 mg/kg every week, every two weeks or every three weeks, of the subject body weight. For example, dosages can be 1 mg/kg body weight or 10 mg/kg body weight every week, every two weeks or every three weeks or within the range of 1-10 mg/kg every week, every two weeks or every three weeks. In one embodiment, a single dosage of antibody ranges from 0.1-10,000 micrograms per kg body weight. In one embodiment, antibody concentrations in a carrier range from 0.2 to 2000 micrograms per delivered milliliter. An exemplary treatment regime entails administration once per every two weeks or once a month or once every 3 to 6 months. Heterodimeric trivalent/tetravalent multispecific antibodies may be administered on multiple occasions. Intervals between single dosages can be hourly, daily, weekly, monthly or yearly. Intervals can also be irregular as indicated by measuring blood levels of the antibody in the subject. In some methods, dosage is adjusted to achieve a serum antibody concentration in the subject of from about 75 μg/mL to about 125 μg/mL, 100 μg/mL to about 150 μg/mL, from about 125 μg/mL to about 175 μg/mL, or from about 150 μg/mL to about 200 μg/mL. Alternatively, heterodimeric trivalent/tetravalent multispecific antibodies can be administered as a sustained release formulation, in which case less frequent administration is required. Dosage and frequency vary depending on the half-life of the antibody in the subject. The dosage and frequency of administration can vary depending on whether the treatment is prophylactic or therapeutic. In prophylactic applications, a relatively low dosage is administered at relatively infrequent intervals over a long period of time. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

Toxicity. Optimally, an effective amount (e.g., dose) of heterodimeric trivalent/tetravalent multispecific antibody described herein will provide therapeutic benefit without causing substantial toxicity to the subject. Toxicity of the heterodimeric trivalent/tetravalent multispecific antibody described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the heterodimeric trivalent/tetravalent multispecific antibody described herein lies within a range of circulating concentrations that include the effective dose with little or no toxicity. The dosage can vary within this range depending upon the dosage form employed and the route of administration utilized. The exact formulation, route of administration and dosage can be chosen by the individual physician in view of the subject's condition. See, e.g., Fingl et al., In: The Pharmacological Basis of Therapeutics, Ch. 1 (1975).

### Formulations of Pharmaceutical Compositions

Formulations of Pharmaceutical Compositions. According to the methods of the present technology, the heterodimeric trivalent/tetravalent multispecific antibodies can be incorporated into pharmaceutical compositions suitable for administration. The pharmaceutical compositions generally comprise recombinant or substantially purified antibody and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the antibody compositions (See, e.g., Remington's Pharmaceutical Sciences, Mack Publishing Co., Easton, Pa. 18^{th} ed., 1990). The pharmaceutical compositions are generally formulated as sterile, substantially isotonic and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, diluents and reagents, are used interchangeably and represent that the materials are capable of administration to a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. “Pharmaceutically-acceptable salts and esters” means salts and esters that are pharmaceutically-acceptable and have the desired pharmacological properties. Such salts include salts that can be formed where acidic protons present in the composition are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically-acceptable esters include esters formed from carboxy, sulfonyloxy, and phosphonoxy groups present in the heterodimeric trivalent/tetravalent multispecific antibody, e.g., C1-6 alkyl esters. When there are two acidic groups present, a pharmaceutically-acceptable salt or ester can be a mono-acid-mono-salt or ester or a di-salt or ester; and similarly where there are more than two acidic groups present, some or all of such groups can be salified or esterified. A heterodimeric trivalent/tetravalent multispecific antibody named in this technology can be present in unsalified or unesterified form, or in salified and/or esterified form, and the naming of such heterodimeric trivalent/tetravalent multispecific antibody is intended to include both the original (unsalified and unesterified) compound and its pharmaceutically-acceptable salts and esters. Also, certain embodiments of the present technology can be present in more than one stereoisomeric form, and the naming of such heterodimeric trivalent/tetravalent multispecific antibody is intended to include all single stereoisomers and all mixtures (whether racemic or otherwise) of such stereoisomers. A person of ordinary skill in the art, would have no difficulty determining the appropriate timing, sequence and dosages of administration for particular drugs and compositions of the present technology.

Examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the heterodimeric trivalent/tetravalent multispecific antibody, use thereof in the compositions is contemplated. Supplementary active compounds can also be incorporated into the compositions.

A pharmaceutical composition of the present technology is formulated to be compatible with its intended route of administration. The heterodimeric trivalent/tetravalent multispecific antibody compositions of the present technology can be administered by parenteral, topical, intravenous, oral, subcutaneous, intraarterial, intradermal, transdermal, rectal, intracranial, intrathecal, intraperitoneal, intranasal; or intramuscular routes, or as inhalants. The heterodimeric trivalent/tetravalent multispecific antibody can optionally be administered in combination with other agents that are at least partly effective in treating a disease or medical condition described herein.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be desirable to include isotonic compounds, e.g., sugars, polyalcohols such as manitol, sorbitol, sodium chloride in the composition. Prolonged absorption of the injectable compositions can be brought about by including in the composition a compound which delays absorption, e.g., aluminum monostearate and gelatin.

Sterile injectable solutions can be prepared by incorporating a heterodimeric trivalent/tetravalent multispecific antibody of the present technology in the required amount in an appropriate solvent with one or a combination of ingredients enumerated above, as required, followed by filtered sterilization. Generally, dispersions are prepared by incorporating the heterodimeric trivalent/tetravalent multispecific antibody into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The antibodies of the present technology can be administered in the form of a depot injection or implant preparation which can be formulated in such a manner as to permit a sustained or pulsatile release of the active ingredient.

Oral compositions generally include an inert diluent or an edible carrier. They can be enclosed in gelatin capsules or compressed into tablets. For the purpose of oral therapeutic administration, the heterodimeric trivalent/tetravalent multispecific antibody can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the heterodimeric trivalent/tetravalent multispecific antibody is delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the heterodimeric trivalent/tetravalent multispecific antibody is formulated into ointments, salves, gels, or creams as generally known in the art.

The heterodimeric trivalent/tetravalent multispecific antibody can also be prepared as pharmaceutical compositions in the form of suppositories (e.g., with conventional suppository bases such as cocoa butter and other glycerides) or retention enemas for rectal delivery.

In one embodiment, the heterodimeric trivalent/tetravalent multispecific antibody is prepared with carriers that will protect the heterodimeric trivalent/tetravalent multispecific antibody against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically-acceptable carriers. These can be prepared according to methods known to those skilled in the art, e.g., as described in U.S. Pat. No. 4,522,811.

### Kits

The present technology provides kits for the detection and/or treatment of cancer, comprising at least one heterodimeric trivalent/tetravalent multispecific antibody composition described herein, or a functional variant (e.g., substitutional variant) thereof. Optionally, the above described components of the kits of the present technology are packed in suitable containers and labeled for diagnosis and/or treatment of cancer. The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kits are useful for detecting the presence of a target antigen in a biological sample, e.g., any body fluid including, but not limited to, e.g., serum, plasma, lymph, cystic fluid, urine, stool, cerebrospinal fluid, ascitic fluid or blood and including biopsy samples of body tissue. For example, the kit can comprise: one or more heterodimeric trivalent/tetravalent multispecific antibodies of the present technology capable of binding a target antigen in a biological sample; means for determining the amount of the target antigen in the sample; and means for comparing the amount of the immunoreactive target antigen in the sample with a standard. One or more of the heterodimeric trivalent/tetravalent multispecific antibodies may be labeled. The kit components, (e.g., reagents) can be packaged in a suitable container. The kit can further comprise instructions for using the kit to detect the immunoreactive target antigen.

For antibody-based kits, the kit can comprise, e.g., 1) a first antibody, e.g. a humanized, or chimeric heterodimeric trivalent/tetravalent multispecific antibody of the present technology, attached to a solid support, which binds to a target antigen; and, optionally; 2) a second, different antibody which binds to either the target antigen or to the first antibody, and is conjugated to a detectable label.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit, e.g., for detection of a target antigen in vitro or in vivo, or for treatment of cancer in a subject in need thereof. In certain embodiments, the use of the reagents can be according to the methods of the present technology.

### EXAMPLES

The present technology is further illustrated by the following Examples, which should not be construed as limiting in any way.

### Example 1: Materials and Methods

Protein production. All proteins were expressed using the expi293 expression system (Thermo Fisher Scientific, Waltham Mass.) according to manufacturer's instructions. Briefly, maxiprepped plasmids containing each antibody were diluted and incubated with expifectamine for 20 min before being added to expi293s in shaker flasks. Cells were incubated for 4 days or until cell viability dropped <70%, whichever came first. IgG-based proteins were purified over a protein A column using a GE P920 AKTA FPLC and eluted using 50 mM Citric acid. The BiTE was purified using prepacked Ni²⁺NTA columns (GE) and eluted using a 250 mM imidazole buffer. All proteins were run on SEC-HPLC to validate their size and quantify their purity.

Heterodimerization. Heterodimerization was achieved using Fab Arm Exchange (FAE). Briefly, K409R and F405L mutations were placed in the Fc regions of each reciprocal pair of IgG or IgG-[L]-scFv bispecific antibodies to be heterodimerized. Paired homodimers were then mixed at 3 different molar rations (1:1, 1.2:1 and 1:1.2) and incubated in reducing conditions for 5 hrs at 30° C. before being dialyzed overnight at room temperature in sodium citrate buffer (pH 8.2). After an initial overnight dialysis, samples were moved to 4° C. for another 24 hrs before being analyzed by SEC-HPLC and CZE chromatography to assess heterodimerization yields. In all experiments the 1:1 ratio was used, after validating its purity was optimal.

Cell lines. EL.4 cells were obtained from ATCC. M14 cells were obtained from ATCC and transfected with luciferase prior to use in all assays. IMR32 cells were obtained from ATCC and transfected with luciferase prior to use in all assays. Molm13-fluc cells were a gift from the Brentjens lab. Naïve T-cells were purified from PBMCs using the Dynabeads™ Untouched™ human T cells kit, according to manufacturer's protocol. Activated T cells were generated by using CD3/CD28 dynabeads and 30U/ml of human IL-2. T-cells were stimulated twice, at day 0 and day 7, and used in cytotoxicity, cell binding or conjugate assays day 15-18 of culture.

Cell binding FACS. For cell binding assays, 1M cells were incubated with 5 pmol of antibody for 30 min at 4° C., followed by either an anti-human Fc secondary or an anti-3F8 or anti-OKT3 idiotype antibody (5 pmol) and the corresponding anti-Fc secondary (anti-rat APC or anti-mouse PE, respectively). Samples were acquired using a FACSCalibur and analyzed by FlowJo.

Affinity Measurements. Binding kinetics were evaluated using SPR (GE, Biacore T200). Briefly, chips were coated with GD2, CD33 or huCD3de antigen and a titration series of each bispecific antibody were flowed over them. Binding affinities were calculated using a two-state reaction model.

Cytotoxicity measurements. Cytotoxicity was evaluated using a 4 hr ⁵¹Cr release assay. Briefly, 1M target cells were incubated with 100 μCi of activity and incubated with activated human T cells (10:1 E:T) and serially titrated bispecific antibody. Released ⁵¹Cr was measured using a gamma counter.

Animal Models. All experiments have been conducted in accordance with and approved by the Institutional Animal Care and Use Committee in MSKCC. Two mouse models were used: (1) a humanized immunodeficient xenograft model (huDKO) and (2) a transgenic huCD3e-expressing syngeneic model (huCD3e-tg). Briefly, huDKO (Balb/C IL2rg^{-/-}, Rag2^{-/-}) mice were implanted subcutaneously with 2M M14 melanoma cells. After 5-15 days, mice were treated with intravenous activated human T cells (20-40M/dose), intravenous bispecific antibody (25 pmol/dose) and subcutaneous IL-2 (100U/dose) for three weeks. For huCD3e-tg (C57BL/6) mice were implanted subcutaneously with EL.4 lymphoma cells. After 7 days, mice were treated intravenous bispecific antibody (25 pmol/dose) for three weeks. For BiTEs, either 7 pmol or 350 pmol were administered daily for 3 weeks. Weights and tumor volumes were measured once per week and overall mouse health was evaluated at least 3-times per week. Mice were sacrificed if tumor volumes reached 1.5-2.0 cm³ volumes. No toxicities were seen during treatment of any mice.

Conjugate formation. For conjugate assays, T cells were labeled with CFSE (2.5 μM) and M14 melanoma cells were labeled with CTV (2.5 μM). 50 M/ml cells were incubated with dye for 5 min at room temperature, followed by the addition of 30 ml of complete RPMI (supplemented with 10% fetal calf serum (heat inactivated), 2 mM glutamine and 1% P/S) and incubated at 37° C. for 20 min. Cells were pelleted and washed with complete medium twice before being added antibodies or cells. Labeled cells were mixed at a 1:5 ratio (E:T) along with serially titrated bispecific antibody, in duplicate. After 30 min, cells were fixed with a final concentration of 2% PFA (10 min, RT) and washed in 5 ml of PBS. Cells were acquired using a BD LSR Fortessa and analyzed using Flowjo.

Activation assay. Purified naïve T cells were incubated with M14 melanoma cells (10:1 E:T) and serially titrated bispecific antibody, in duplicate. After 24 hrs supernatant was collected and frozen at −80° C. Cells were then stained with antibodies against CD4, CD8, CD45, and CD69 to assess the CD69 upregulation. For the 96 hr assay, T cells were first labeled with 2.504 of CTV. After 96 hrs cells were stained with antibodies against CD4, CD8, CD45 and CD25 to assess CD25 upregulation and CTV dilution.

Cytokine Assay. Frozen supernatant from the activation assay (24 hr) was used to quantify cytokine production after 24 hrs of coculture. IL-2, IFNγ, IL-10, IL-6 and TNFα were measured with the 5-plex legend plex system according to manufacturer guidelines.

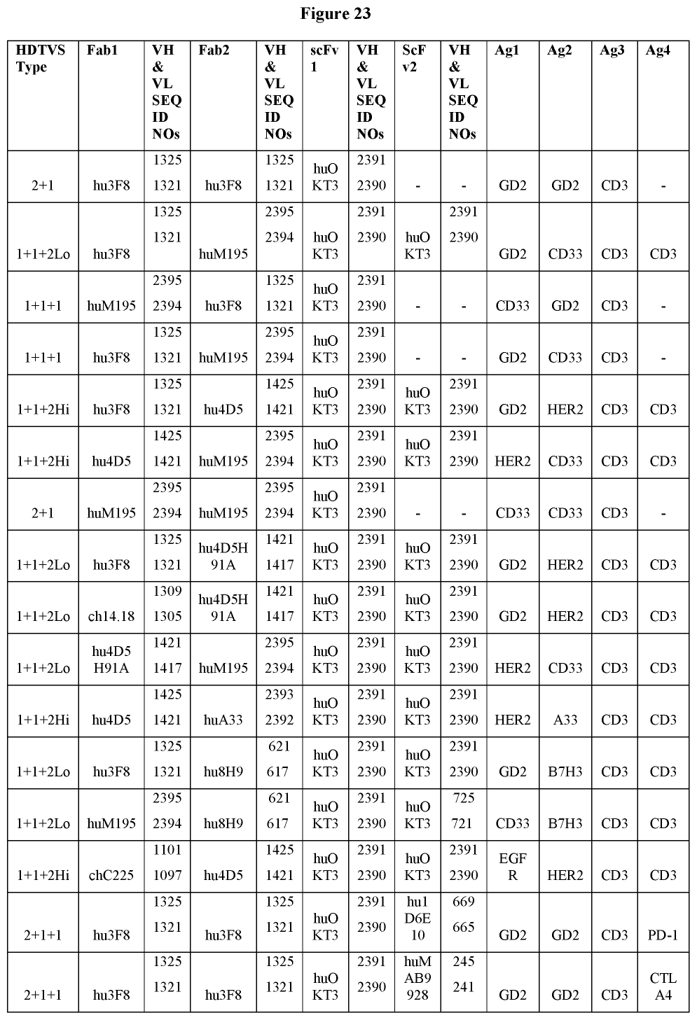

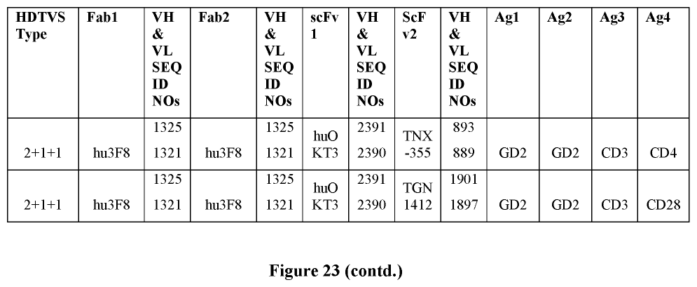

FIG. 23 provides a summary of the various HDTVS antibodies tested in the Examples disclosed herein. The table summarizes all successfully produced HDTVS formatted multi-specific antibodies across a variety of antigen models. All clones were expressed in Expi293 cells and heterodimerized using the controlled Fab Arm Exchange method. HDTVS type displays the category of each clone. Fab 1 and scFv 1 (and corresponding Ag1 and Ag3) are attached in a cis-orientation on one heavy chain (linked by the light chain of Fab) while Fab 2 and scFv 2 (and corresponding Ag2 and Ag4) are on a separate heavy chain molecule in a cis-orientation (linked by the light chain of Fab).

Sequences. The amino acid sequences utilized in the Examples are provided below:

Anti-HER2
LC(VL-CL-scFv)
(SEQ ID NO: 2353)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECTSGGGGSGGGGSGGGGSQVQLVQSGGGVVQPGRSLR
LSCKASGYTFTRYTIVIRWVRQAPGKCLEWIGYINPSRGYTNYNQKFKDR
FTISRDNSKNTAFLQMDSLRPEDTGVYFCARYYDDHYSLDYWGQGTPVTV
SSGGGGSGGGGSGGGGSGGGGSGGGGSGGGGSDIQMTQSPSSLSASVGDR
VTITCSASSSVSYMNWYQQTPGKAPKRWIYDTSKLASGVPSRFSGSGSGT
DYTFTISSLQPEDIATYYCQQWSSNPFTFGCGTKLQITR
HC(VH-CH1-CH2-CH3, N297A, K322A):
(SEQ ID NO: 2354)
EVQLVESGGGLVQPGGSLRLSCAASGFNIKDTYIHWVRQAPGKGLEWVAR
TYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYA
STYRVVSVLTVLHQDWLNGKEYKCAVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
HC(VH-CH1-CH2-CH3, N297A, K322A, F405L):
(SEQ ID NO: 2355)
EVQLVESGGGLVQPGGSLRLSCAASGFNIKDTYIHWVRQAPGKGLEWVAR
TYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYA
STYRVVSVLTVLHQDWLNGKEYKCAVSNKALPAPIEKTISKAKGQPREPQ
VYTLPPSRDELTKNQVSLTCLVKGFYPSDIAVEWESNGQPENNYKTTPPV
LDSDGSFLLYSKLTVDKSRWQQGNVFSCSVMHEALHNHYTQKSLSLSPGK
HC(VH-CH1-CH2-CH3, N297A, K322A, K409R):
(SEQ ID NO: 2356)
EVQLVESGGGLVQPGGSLRLSCAASGFNIKDTYIHWVRQAPGKGLEWVAR
TYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYA
STYRVVSVLTVLHQDWLNGKEYKCAVSNKALPAPIEKTISKAKGQPREPQ
VYTLPPSRDELTKNQVSLTCLVKGFYPSDIAVEWESNGQPENNYKTTPPV
LDSDGSFFLYSRLTVDKSRWQQGNVFScSVMHEALHNHYTQKSLSLSPGK
Anti-GD2
LC(VL-CL-scFv):
(SEQ ID NO: 2357)
EIVMTQTPATLSVSAGERVTITCKASQSVSNDVTWYQQKPGQAPRLLIYS
ASNRYSGVPARFSGSGYGTEFTFTISSVQSEDFAVYFCQQDYSSFGQGTK
LEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSS
PVTKSFNRGECTSGGGGSGGGGSGGGGSQVQLVQSGGGVVQPGRSLRLSC
KASGYTFTRYTMHWVRQAPGKCLEWIGYINPSRGYTNYNQKFKDRFTISR
DNSKNTAFLQMDSLRPEDTGVYFCARYYDDHYSLDYWGQGTPVTVSSGGG
GSGGGGSGGGGSGGGGSGGGGSGGGGSDIQMTQSPSSLSASVGDRVTITC
SASSSVSYMNWYQQTPGKAPKRWIYDTSKLASGVPSRFSGSGSGTDYTFT
ISSLQPEDIATYYCQQWSSNPFTFGCGTKLQITR
LC(VL-CL):
(SEQ ID NO: 2358)
EIVMTQTPATLSVSAGERVTITCKASQSVSNDVTWYQQKPGQAPRLLIYS
ASNRYSGVPARFSGSGYGTEFTFTISSVQSEDFAVYFCQQDYSSFGQGTK
LEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSS
PVTKSFNRGEC
HC(VH-CH1-CH2-CH3, N297A, K322A):
(SEQ ID NO: 2359)
QVQLVESGPGVVQPGRSLRISCAVSGFSVTNYGVHWVRQPPGKGLEWLGV
IWAGGITNYNSAFMSRLTISKDNSKNTVYLQMNSLRAEDTAMYYCASRGG
HYGYALDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYAS
TYRVVSVLTVLHQDWLNGKEYKCAVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFScSVMHEALHNHYTQKSLSLSPGK
HC(VH-CH1-CH2-CH3, N297A, K322A, F405L):
(SEQ ID NO: 2360)
QVQLVESGPGVVQPGRSLRISCAVSGFSVTNYGVHWVRQPPGKGLEWLGV
IWAGGITNYNSAFMSRLTISKDNSKNTVYLQMNSLRAEDTAMYYCASRGG
HYGYALDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYAS
TYRVVSVLTVLHQDWLNGKEYKCAVSNKALPAPIEKTISKAKGQPREPQV
YTLPPSRDELTKNQVSLTCLVKGFYPSDIAVEWESNGQPENNYKTTPPVL
DSDGSFLLYSKLTVDKSRWQQGNVFSCSVMHEALHNHYTQKSLSLSPGK
HC(VH-CH1-CH2-CH3, N297A, K322A, K409R):
(SEQ ID NO: 2361)
QVQLVESGPGVVQPGRSLRISCAVSGFSVTNYGVHWVRQPPGKGLEWLGV
IWAGGITNYNSAFMSRLTISKDNSKNTVYLQMNSLRAEDTAMYYCASRGG
HYGYALDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYAS
TYRVVSVLTVLHQDWLNGKEYKCAVSNKALPAPIEKTISKAKGQPREPQV
YTLPPSRDELTKNQVSLTCLVKGFYPSDIAVEWESNGQPENNYKTTPPVL
DSDGSFFLYSRLTVDKSRWQQGNVFSCSVMHEALHNHYTQKSLSLSPGK
Anti-GD2(2)
LC(VL-CL-scFv):
(SEQ ID NO: 2362)
KIVMTQTPATLSVSAGERVTITCKASQSVSNHVTWYQQKPGQAPRLLIYS
ASNRYSGVPARFSGSGYGTEFTFTISSVQSEDFAVYFCQQDYSSFGQGTK
LEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSS
PVTKSFNRGECTSGGGGSGGGGSGGGGSQVQLVQSGGGVVQPGRSLRLSC
KASGYTFTRYTMHWVRQAPGKCLEWIGYINPSRGYTNYNQKFKDRFTISR
DNSKNTAFLQMDSLRPEDTGVYFCARYYDDHYSLDYWGQGTPVTVSSGGG
GSGGGGSGGGGSGGGGSGGGGSGGGGSDIQMTQSPSSLSASVGDRVTITC
SASSSVSYMNWYQQTPGKAPKRWIYDTSKLASGVPSRFSGSGSGTDYTFT
ISSLQPEDIATYYCQQWSSNPFTFGCGTKLQITR
LC(VL-CL):
(SEQ ID NO: 2363)
KIVMTQTPATLSVSAGERVTITCKASQSVSNHVTWYQQKPGQAPRLLIYS
ASNRYSGVPARFSGSGYGTEFTFTISSVQSEDFAVYFCQQDYSSFGQGTK
LEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSS
PVTKSFNRGEC
HC(VH-CH1-CH2-CH3, N297A, K322A):
(SEQ ID NO: 2364)
QVQLVESGPGVVQPGRSLRISCAVSGFSVTNYGVHWVRQPPGKGLEWLGV
IWAGGITNYNSAFMSRLTISKDNSKNTVYLQMNSLRAEDTAMYYCASRGG
HYGYALDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYAS
TYRVVSVLTVLHQDWLNGKEYKCAVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
HC(VH-CH1-CH2-CH3, N297A, K322A, F405L):
(SEQ ID NO: 2365)
QVQLVESGPGVVQPGRSLRISCAVSGFSVTNYGVHWVRQPPGKGLEWLGV
IWAGGITNYNSAFMSRLTISKDNSKNTVYLQMNSLRAEDTAMYYCASRGG
HYGYALDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYAS
TYRVVSVLTVLHQDWLNGKEYKCAVSNKALPAPIEKTISKAKGQPREPQV
YTLPPSRDELTKNQVSLTCLVKGFYPSDIAVEWESNGQPENNYKTTPPVL
DSDGSFLLYSKLTVDKSRWQQGNVFSCSVMHEALHNHYTQKSLSLSPGK
HC(VH-CH1-CH2-CH3, N297A, K322A, K409R):
(SEQ ID NO: 2366)
QVQLVESGPGVVQPGRSLRISCAVSGFSVTNYGVHWVRQPPGKGLEWLGV
IWAGGITNYNSAFMSRLTISKDNSKNTVYLQMNSLRAEDTAMYYCASRGG
HYGYALDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYAS
TYRVVSVLTVLHQDWLNGKEYKCAVSNKALPAPIEKTISKAKGQPREPQV
YTLPPSRDELTKNQVSLTCLVKGFYPSDIAVEWESNGQPENNYKTTPPVL
DSDGSFFLYSRLTVDKSRWQQGNVFSCSVMHEALHNHYTQKSLSLSPGK
Anti-GD2(3)
LC(VL-CL-scFv):
(SEQ ID NO: 2367)
EIVMTQSPATLSVSPGERATLSCRSSQSLVHRNGNTYLHWYLQKPGQSPK
LLIHKVSNRFSGVPDRFSGSGSGTDFTLKISRVEAEDLGVYFCSQSTHVP
PLTFGAGTKLELKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGECTSGGGGSGGGGSGGGGSQVQLVQSGGGVVQ
PGRSLRLSCKASGYTFTRYTMHWVRQAPGKCLEWIGYINPSRGYTNYNQK
FKDRFTISRDNSKNTAFLQMDSLRPEDTGVYFCARYYDDHYSLDYWGQGT
PVTVSSGGGGSGGGGSGGGGSGGGGSGGGGSGGGGSDIQMTQSPSSLSAS
VGDRVTITCSASSSVSYMNWYQQTPGKAPKRWIYDTSKLASGVPSRFSGS
GSGTDYTFTISSLQPEDIATYYCQQWSSNPFTFGCGTKLQITR
LC(VL-CL):
(SEQ ID NO: 2368)
EIVMTQSPATLSVSPGERATLSCRSSQSLVHRNGNTYLHWYLQKPGQSPK
LLIHKVSNRFSGVPDRFSGSGSGTDFTLKISRVEAEDLGVYFCSQSTHVP
PLTFGAGTKLELKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC
HC(VH-CH1-CH2-CH3, N297A, K322A):
(SEQ ID NO: 2369)
EVQLLQSGPELEKPGASVMISCKASGSSFTGYNMNWVRQNIGKSLEWIGA
IDPYYGGTSYNQKFKGRATLTVDKSSSTAYMHLKSLTSEDSAVYYCVSGM
EYWGQGTSVTVSSASTKGPSVFPLAPSSKSTSGGTAALGCLVKDYFPEPV
TVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYASTYRVVS
VLTVLHQDWLNGKEYKCAVSNKALPAPIEKTISKAKGQPREPQVYTLPPS
RDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
HC(VH-CH1-CH2-CH3, N297A, K322A, F405L):
(SEQ ID NO: 2370)
EVQLLQSGPELEKPGASVMISCKASGSSFTGYNMNWVRQNIGKSLEWIGA
IDPYYGGTSYNQKFKGRATLTVDKSSSTAYMHLKSLTSEDSAVYYCVSGM
EYWGQGTSVTVSSASTKGPSVFPLAPSSKSTSGGTAALGCLVKDYFPEPV
TVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYASTYRVVS
VLTVLHQDWLNGKEYKCAVSNKALPAPIEKTISKAKGQPREPQVYTLPPS
RDELTKNQVSLTCLVKGFYPSDIAVEWESNGQPENNYKTTPPVLDSDGSF
LLYSKLTVDKSRWQQGNVFScSVMHEALHNHYTQKSLSLSPGK
HC(VH-CH1-CH2-CH3, N297A, K322A, K409R):
(SEQ ID NO: 2371)
EVQLLQSGPELEKPGASVMISCKASGSSFTGYNMNWVRQNIGKSLEWIGA
IDPYYGGTSYNQKFKGRATLTVDKSSSTAYMHLKSLTSEDSAVYYCVSGM
EYWGQGTSVTVSSASTKGPSVFPLAPSSKSTSGGTAALGCLVKDYFPEPV
TVSWNSGALTSGVHTFPAVLQSSGLYSLSSVVTVPSSSLGTQTYICNVNH
KPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYASTYRVVS
VLTVLHQDWLNGKEYKCAVSNKALPAPIEKTISKAKGQPREPQVYTLPPS
RDELTKNQVSLTCLVKGFYPSDIAVEWESNGQPENNYKTTPPVLDSDGSF
FLYSRLTVDKSRWQQGNVFSCSVMHEALHNHYTQKSLSLSPGK
Anti-CD33
LC(VL-CL-scFv):
(SEQ ID NO: 2372)
EIVLTQSPATLSVSLGERATISCRASESVDNYGISFMNWFQQKPGQPPRL
LIYAASNQGSGVPARFSGSGPGTDFTLTISSMEPEDFAMYFCQQSKEVPW
TFGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGECTSGGGGSGGGGSGGGGSQVQLVQSGGGVVQPG
RSLRLSCKASGYTFTRYTMHWVRQAPGKCLEWIGYINPSRGYTNYNQKFK
DRFTISRDNSKNTAFLQMDSLRPEDTGVYFCARYYDDHYSLDYWGQGTPV
TVSSGGGGSGGGGSGGGGSGGGGSGGGGSGGGGSDIQMTQSPSSLSASVG
DRVTITCSASSSVSYMNWYQQTPGKAPKRWIYDTSKLASGVPSRFSGSGS
GTDYTFTISSLQPEDIATYYCQQWSSNPFTFGCGTKLQITR
LC(VL-CL):
(SEQ ID NO: 2373)
EIVLTQSPATLSVSLGERATISCRASESVDNYGISFMNWFQQKPGQPPRL
LIYAASNQGSGVPARFSGSGPGTDFTLTISSMEPEDFAMYFCQQSKEVPW
TFGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
HC(VH-CH1-CH2-CH3, N297A, K322A):
(SEQ ID NO: 2374)
EVQLVQSGPEVVKPGASVKISCKASGYTFTDYNMHWVRQAHGQSLEWIGY
IYPYNGGTGYNQKFKSRATLTVDNSASTAYMEVSSLRSEDTAVYYCARGR
PAMDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYASTYR
VVSVLTVLHQDWLNGKEYKCAVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
HC(VH-CH1-CH2-CH3, N297A, K322A, F405L):
(SEQ ID NO: 2375)
EVQLVQSGPEVVKPGASVKISCKASGYTFTDYNMHWVRQAHGQSLEWIGY
IYPYNGGTGYNQKFKSRATLTVDNSASTAYMEVSSLRSEDTAVYYCARGR
PAMDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYASTYR
VVSVLTVLHQDWLNGKEYKCAVSNKALPAPIEKTISKAKGQPREPQVYTL
PPSRDELTKNQVSLTCLVKGFYPSDIAVEWESNGQPENNYKTTPPVLDSD
GSFLLYSKLTVDKSRWQQGNVFSCSVMHEALHNHYTQKSLSLSPGK
HC(VH-CH1-CH2-CH3, N297A, K322A, K409R):
(SEQ ID NO: 2376)
EVQLVQSGPEVVKPGASVKISCKASGYTFTDYNMHWVRQAHGQSLEWIGY
IYPYNGGTGYNQKFKSRATLTVDNSASTAYMEVSSLRSEDTAVYYCARGR
PAMDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYASTYR
VVSVLTVLHQDWLNGKEYKCAVSNKALPAPIEKTISKAKGQPREPQVYTL
PPSRDELTKNQVSLTCLVKGFYPSDIAVEWESNGQPENNYKTTPPVLDSD
GSFFLYSRLTVDKSRWQQGNVFSCSVMHEALHNHYTQKSLSLSPGK
Anti-CD3
LC(VL-CL):
(SEQ ID NO: 2377)
DIQMTQSPSSLSASVGDRVTITCSASSSVSYMNWYQQTPGKAPKRWIYDT
SKLASGVPSRFSGSGSGTDYTFTISSLQPEDIATYYCQQWSSNPFTFGQG
TKLQITRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
HC(VH-CH1-CH2-CH3, N297A, K322A):
(SEQ ID NO: 2378)
QVQLVQSGGGVVQPGRSLRLSCKASGYTFTRYTMHWVRQAPGKGLEWIGY
INPSRGYTNYNQKFKDRFTISRDNSKNTAFLQMDSLRPEDTGVYFCARYY
DDHYSLDYWGQGTPVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYAS
TYRVVSVLTVLHQDWLNGKEYKCAVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFScSVMHEALHNHYTQKSLSLSPGK
HC(VH-CH1-CH2-CH3, N297A, K322A, F405L):
(SEQ ID NO: 2379)
QVQLVQSGGGVVQPGRSLRLSCKASGYTFTRYTMHWVRQAPGKGLEWIGY
INPSRGYTNYNQKFKDRFTISRDNSKNTAFLQMDSLRPEDTGVYFCARYY
DDHYSLDYWGQGTPVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYAS
TYRVVSVLTVLHQDWLNGKEYKCAVSNKALPAPIEKTISKAKGQPREPQV
YTLPPSRDELTKNQVSLTCLVKGFYPSDIAVEWESNGQPENNYKTTPPVL
DSDGSFLLYSKLTVDKSRWQQGNVFSCSVMHEALHNHYTQKSLSLSPGK
HC(VH-CH1-CH2-CH3, N297A, K322A, K409R):
(SEQ ID NO: 2380)
QVQLVQSGGGVVQPGRSLRLSCKASGYTFTRYTMHWVRQAPGKGLEWIGY
INPSRGYTNYNQKFKDRFTISRDNSKNTAFLQMDSLRPEDTGVYFCARYY
DDHYSLDYWGQGTPVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYAS
TYRVVSVLTVLHQDWLNGKEYKCAVSNKALPAPIEKTISKAKGQPREPQV
YTLPPSRDELTKNQVSLTCLVKGFYPSDIAVEWESNGQPENNYKTTPPVL
DSDGSFFLYSRLTVDKSRWQQGNVFSCSVMHEALHNHYTQKSLSLSPGK
huOKT3-VL
(SEQ ID NO: 2390)
DIQMTQSPSSLSASVGDRVTITCSASSSVSYMNWYQQTPGKAPKRWIYDT
SKLASGVPSRFSGSGSGTDYTFTISSLQPEDIATYYCQQWSSNPFTFGCG
TKLQIT
huOKT3-VH
(SEQ ID NO: 2391)
QVQLVQSGGGVVQPGRSLRLSCKASGYTFTRYTMHWVRQAPGKCLEWIGY
INPSRGYTNYNQKFKDRFTISRDNSKNTAFLQMDSLRPEDTGVYFCARYY
DDHYSLDYWGQGTPVTVSS
huA33-VL
(SEQ ID NO: 2392)
DIQMTQSQSSLSTSVGDRVTITCKASQNVRTVVAWYQQKPGKSPKTLIYL
ASNRHTGVPSRFSGSGSGTEFTLTISNVQPEDFADYFCLQHWSYPLTFGS
GTKLEIK
huA33-VH
(SEQ ID NO: 2393)
EVQLVESGGGLVKPGGSLRLSCAASGFAFSTYDMSWVRQAPGKRLEWVAT
ISSGGSYTYYLDSVKGRFTISRDNAKNSLYLQMNSLRAEDTAVYYCAPTT
VVPFAYWGQGTLVTVSS
huM195-VL
(SEQ ID NO: 2394)
EIVLTQSPATLSVSLGERATISCRASESVDNYGISFMNWFQQKPGQPPRL
LIYAASNQGSGVPARFSGSGPGTDFTLTISSMEPEDFAMYFCQQSKEVPW
TFGGGTKLEIK
huM195-VH
(SEQ ID NO: 2395)
EVQLVQSGPEVVKPGASVKISCKASGYTFTDYNMHWVRQAHGQSLEWIGY
IYPYNGGTGYNQKFKSRATLTVDNSASTAYMEVSSLRSEDTAVYYCARGR
PAMDYWGQGTLVTVSS

### Example 2: Functionality of Lo1+1+2, Hi1+1+1 and 2+1+1 HDTVS Variants

FIG. 1a shows the basic design strategy of each HDTVS variant compared with the parental 2+2 IgG-[L]-scFv. FIGS. 1b-1g describe each of the three designs in more detail.

The Lo1+1+2 utilizes two different Fab domains that (a) target two distinct antigens within a tumor and (b) have moderate to low binding affinities (e.g. K_{D} 100 nM-100 pM), and two identical scFvs that target an immune cell so as to improve tumor cell specificity. As illustrated in FIG. 1b, this design targets tumors more specifically due to its unexpectedly poor activity when only one of the two Fab domains is engaged with the tumor target (such as when only one of the two Fab domain-specific antigens is expressed). Importantly, when both Fab domains bind their respective tumor targets, normal cytotoxic potency is restored. This allows for improved therapeutic index (or safety) when the target antigens are not unique to the tumor, where each target antigen (but never both) is shared to some extent by normal cells. While a standard BsAb or 2+2 design would harm normal tissues, this Lo1+1+2 design should spare normal tissues that express only one of the two targeted antigens, while maintaining the full potency against a tumor cell that expresses both antigens.

As illustrated in FIG. 1c, the Hi1+1+2 design is capable of recognizing two distinct antigens with equal potency, regardless of simultaneous binding. Since Fab domains of appropriately high affinity (e.g., K_{D}<100 pM) are sufficient to induce potent cytotoxicity even monovalently, two different Fab domains can be used to broaden the tumor cell selectivity and permits targeting of heterogeneous tumors with a single drug.

The 2+1+1 design is capable of improved immune cell interactions by virtue of its dual specificity toward the immune cell, either improving activation or providing more selective activation. As demonstrated herein, the second scFv domain is somewhat dispensable due to the biophysical properties of the IgG-[L]-scFv platform. Thus, using two different scFv domains can provide a greater diversity of interactions than a normal bivalent approach. As illustrated in FIG. 1d, the 2+1+1 design can be used to both improve signaling in a more selective population of immune cells (B1(+)B2(+)) or to enhance activation through colocalization of complementary pairs of receptors. Importantly, the 2+1+1 design can be used to interact with activating receptors and/or inhibitory receptors or antagonistic antibodies that specifically inhibit signaling of certain immune cell pathways, such as blocking PD-1 on T cells while activating through CD3.

The 2+1+1 design takes advantage of the two anti-immune cell binding domains to recruit a broader selection of immune cells (e.g., anti-CD3 for T cells+anti-CD16 for NK cells) or for combinatorial recruitment of payloads with immune cells as theranostics (e.g., anti-CD3 for T cells and anti-BnDOTA for imaging). As illustrated in FIG. 1e, the 2+1+1 design takes advantage of the minimal differences in therapeutic activity between a 2+1 design and a 2+2 design to add a new function, thus broadening the selection of delivered anti-tumor activity to multiple types of immune cells or to chemical or radiological payloads.

The 1+1+1+1 format combines the previous 4 designs to take advantage of all possible combinations. As shown in Figure if, this allows for the combinatorial properties of the 2+1+1 design to be combined with the specificity or selectivity improvements from the Hi1+1+2 and Lo1+1+2 designs.

### Example 3: —Superiority of 2+2 IgG-[L]-scFv Design over BITE and IgG-Het

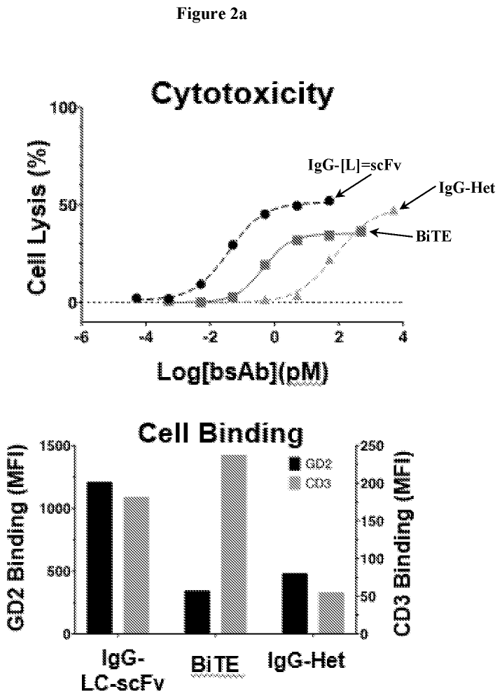

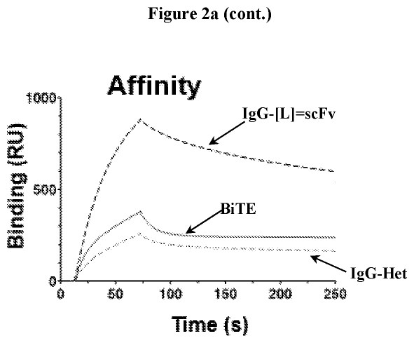

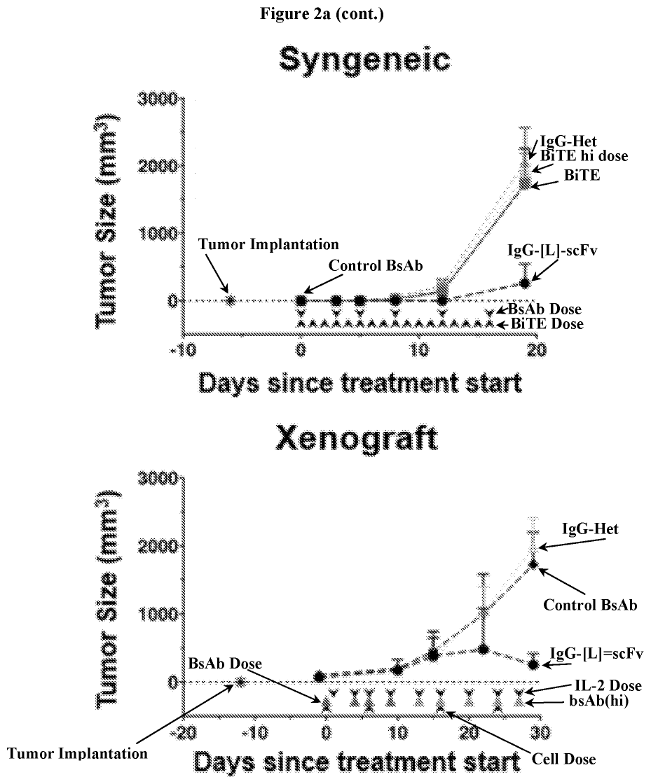

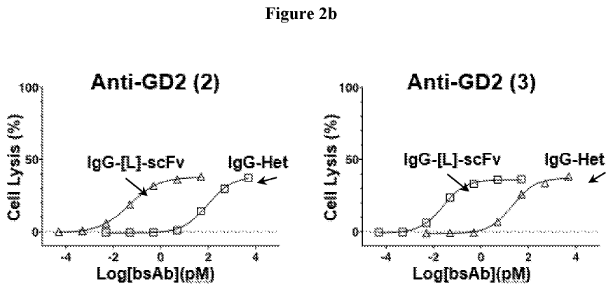

FIG. 2a-2b show the unexpected benefits of the IgG-[L]-scFv (2+2 BsAb) over other common designs such as IgG-Het and BiTE, highlighting both the benefit of having a valency >1 and the structural properties imparted by a Fab/scFv combination. As shown in FIG. 2a, the top panels compare cytotoxicity, cell binding and antigen affinity properties between the IgG-[L]-scFv, IgG-Het and BiTE formats.

The left most panel shows that the 2+2 BsAb achieved nearly 1,000-fold improved cytotoxicity over the 1+1 IgG-Het and >20-fold than the 1+1 BiTE. Measurements were made using a standard four hour ⁵¹Cr release assay using activated human T cells and GD2(+) M14-luciferase cells, with each antibody diluted over 7-logs. The center panel shows the varying levels of antigen binding (GD2 or CD3) between these three formats using GD2(+) M14-luciferase cells or CD3(+) activated human T cells. Cells were stained with each of the three formats and detected using either anti-hu3F8 or anti-huOKT3 idiotypic antibodies. As with the cytotoxicity, the cell binding to both antigens was superior for the 2+2 BsAb due to increased valency. The right panel displays the binding kinetics against the antigen GD2 for each of the three platforms. The 2+2 BsAb exhibited stronger antigen binding over either 1+1 design (BITE or IgG-Het). The bottom panels compare these three constructs in two separate animal models: a huCD3(+) transgenic syngeneic mouse model (left panel) or a humanized immunodeficient xenograft mouse model (right panel). Both models had antibodies injected twice per week and began approximately one week after tumor implantation. Only the 2+2 BsAb was capable of delaying subcutaneous GD2(+) EL.4 tumor growth in the syngeneic model. The 1+1 IgG-Het and the 1+1 BiTE were just as ineffective as the inactive negative control BsAb. Administering the BiTE format daily or at a 10× higher dose level (“hi dose” group, syngeneic mice, FIG. 2a) did not result in any anti-tumor effect. In the xenograft model, where human ATCs and IL-2 were added to support T cell survival in all groups, the 1+1 IgG-Het still failed to show any benefit compared to the control, while the 2+2 BsAb strongly inhibited subcutaneous GD2(+) M14Luc tumors. As show in FIG. 2b, these striking differences in cytotoxicity between the IgG-[L]-scFv and IgG-Het formats were reproducible using two additional anti-GD2 antibodies, suggesting that the effects were not specific to any one GD2 epitope.

These results demonstrate that the HDTVS antibodies disclosed herein are useful in methods for treating a disease or condition, such as cancer.

### Example 4: Characterization of IgG-[L]-scFv HDTVS Variants

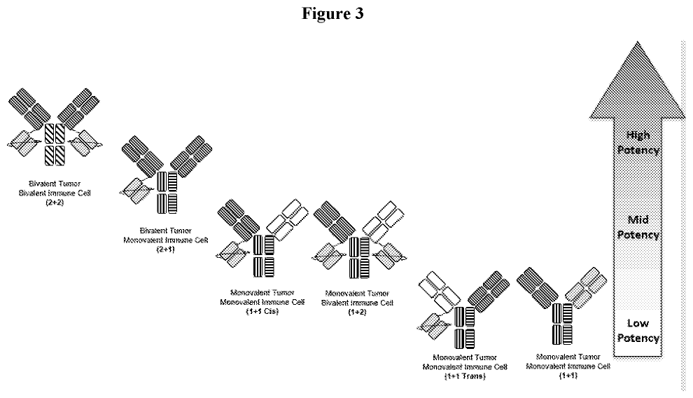

FIG. 3 describes the characterization of the IgG-[L]-scFv platform to identify the necessity and sufficiency of each binding domain as well as their relative impact on overall functional activity. Unexpectedly, the changes in valency did not entirely correlate with changes in functional output, suggesting a preference for tumor binding by the Fab domain over immune cell binding by the scFv domain, as well as a preference for cis-oriented domains over trans-oriented domains.

As illustrated in FIG. 3, the four IgG-[L]-scFv variants display potencies somewhere between the parental 2+2 IgG-[L]-scFv (top left) and the IgG-Het (bottom right). The 2+1 BsAb (second from left) used heterodimerization to remove one of the two immune cell binding scFv domains yet functioned quite similarly to the parental 2+2 BsAb. Neutralization of the second tumor cell binding Fab domain to create a 1+2 BsAb (third from right) reduced the potency further, but unexpectedly additional removal of an scFv domain did not significantly change the potency, as long as the two remaining domains were in a Cis orientation (1+1C, third from left). Neutralization of the second tumor cell binding Fab was achieved by replacing it with a Fab that binds CD33, an antigen not found on tumor cells or T cells. Neutralization/removal of both the tumor binding Fab domain and the T cell engaging scFv domain in a Trans orientation (1+1T, second from right) caused the biggest drop in potency (equivalent to the IgG-Het), even lower than the 1+1C despite equivalent valency. These results demonstrate that orientation or spatial arrangements of the antigen binding domains are important determinants of therapeutic potency.

These results demonstrate that the HDTVS antibodies disclosed herein are useful in methods for treating a disease or condition, such as cancer.

### Example 5: Modifications of the 2+2 IgG-[L]-scFv and Their Relative Binding Activities

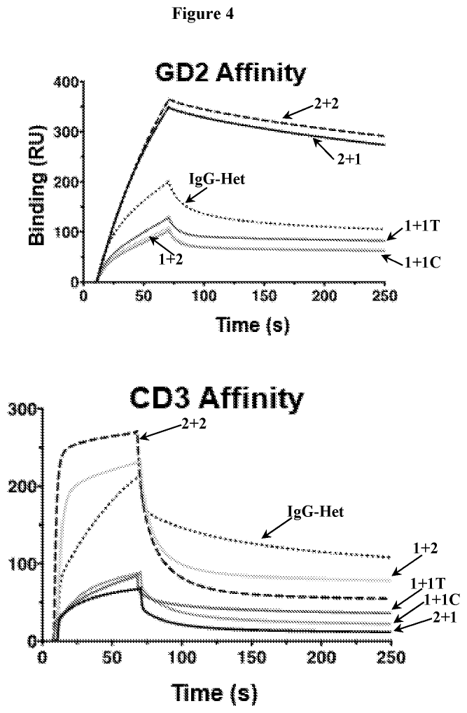

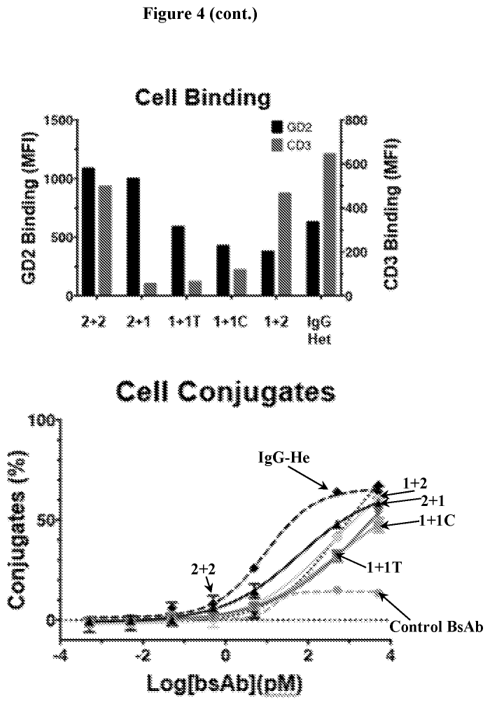

FIG. 4 describes the binding activities of each IgG-[L]-scFv variant, compared to the parental 2(GD2)+2(CD3) BsAb and the IgG-Het. Monovalency towards tumor (e.g. 1+2), was created by changing one of the 2 Fab domains to an irrelevant binder (i.e., a huCD33 targeting Fab). Monovalency (e.g. 2+1) towards T cells is created by removing one of the two scFv domains. As illustrated in FIG. 4, bivalency improves antigen binding over monovalency (upper panels). Surface Plasmon Resonance was used to measure antigen binding kinetics against both GD2 coated chips (upper left) and CD3 coated chips (upper right). Briefly, each BsAb was serially titrated and flowed against each chip. Against GD2, the 2+2 BsAb and 2(GD2)+1(CD3) BsAb showed equivalent binding activities whereas the 1+1C, 1+1T, 1+2 and 1+1 IgG-Het all displayed inferior GD2 binding. Against CD3, the pattern was similar, with bivalency being superior over monovalency, but to a lesser extent (which may be attributable in part to the spatial restrictions of bivalent scFv binding compared to Fab binding). The 2+2 and 1+2 BsAb showed the strongest binding, while the 2+1, 1+1T and 1+1C exhibited inferior binding kinetics. The Fab binding domain of the IgG-Het appeared to show some benefit over a monovalent scFv, but this may result from the more stable sequence of a Fab domain compared with an scFv domain, where CH1/CL interactions are lacking. Compared to SPR, cell binding (measured as described in FIG. 2 but using a standard anti-Fc secondary antibody instead of using anti-idiotypic antibodies) showed similar results (bottom left). GD2 binding (left Y-axis) was the strongest in constructs with bivalency (2+2, 2+1), and less for constructs with monovalency (1+1T, 1+1C, 1+2 and IgG-Het). The same pattern was observed with CD3-specific cell binding (right Y-axis), with 2+2 and 1+2 binding being more effective than 2+1, 1+1T and 1+1C.

Similar to the CD3-specific SPR readings, the IgG-Het showed stronger Fab binding than scFv binding. Conjugate formation between targets and effector cells when mixed together with titrated BsAb (bottom right), showed much smaller differences between IgG-[L]-scFv variants. The 2+2 BsAb showed the most efficient conjugate formation activity, followed by the 2+1 BsAb and then all others (except control). These results demonstrate that after the removal of the second anti-effector cell scFv, all other changes to the IgG-[L]-scFv do not markedly reduce its capacity to conjugate effector target cells together, or that the small differences in cell binding activities do not impact conjugate formation or the stability of conjugate formation.

These results demonstrate that the HDTVS antibodies disclosed herein are useful in methods for treating a disease or condition, such as cancer.

### Example 6: Modifications of the 2+2 IgG-[L]-scFv and their Relative Cytotoxicity

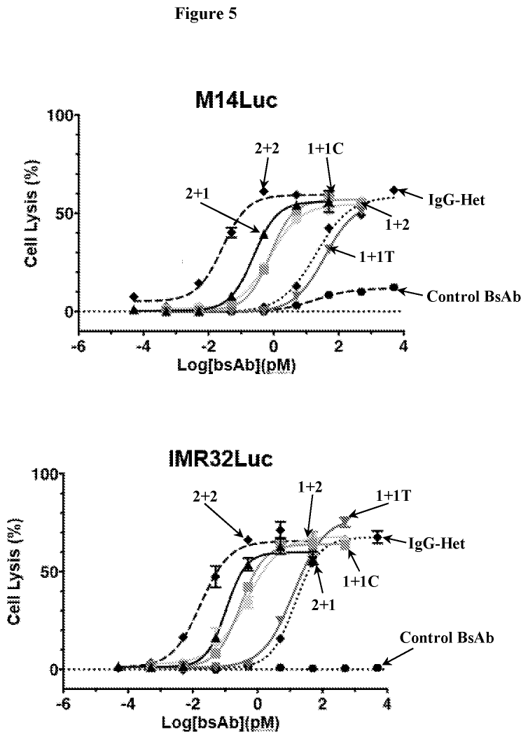

FIG. 5 describes the anti-tumor cytotoxicity of each IgG-[L]-scFv variant in vitro, across two GD2(+) cell lines. As illustrated in FIG. 5 and summarized in TABLE 2, the variants showed a wide range of cytotoxic potency (assays were performed as described in FIG. 2).

TABLE 2

| | K_{D} | | | | Cytotoxic EC50 | |
|---|---|---|---|---|---|---|
| | | Fold | | Fold | | Fold |
| | GD2 | Change | CD3 | Change | EC50 | Change |
| 2 + 2 | 2.8 nM | — | 10 nM | — | 17 fM | — |
| 2 + 1 | 2.5 nM | 0.9 | 310 nM | 30.1 | 106 fM | 6.2 |
| 1 + 1C | 30 nM | 10.9 | 110 nM | 11.0 | 292 fM | 17.2 |
| 1 + 2 | 31 nM | 11.3 | 11 nM | 1.0 | 454 fM | 26.7 |
| 1 + 1H | 31 nM | 11.4 | 70 nM | 6.8 | 14 pM | 823.5 |
| 1 + 1T | 21 nM | 7.7 | 88 nM | 8.5 | 13 pM | 764.7 |

Against both tumor cell lines, the 2+2 BsAb displayed the highest cytotoxic effect, followed by the 2+1 and then both 1+1C and 1+2. Interestingly, the 1+1T and IgG-Het (nearly 1,000-fold worse than 2+2) were nearly identical to each other, suggesting that: the cis-oriented binding domains provide superior killing activity compared to trans-oriented binding domains, and that a 2+1 interaction is superior to a 1+2 interaction. Despite the similarities of both the trans and cis oriented 1+1 variants having identical tumor cell binding, effector cell binding capacities, antigen binding kinetics, and conjugate formation activity, the cis-trans orientations of these two constructs differ substantially in the functional output (50-fold) as measured by in vitro cytotoxicity. This unexpected observation may account for why the 1+2 fails to kill as potently as the 2+1. Without wishing to be bound by theory, it is believed that the 1+2 interaction may be caught between a cis and trans interaction at all times, while the 2+1 is more often in a cis interaction. An alternative possibility is that the tumor-binding Fab domains may be more critical for driving anti-tumor potency.

Additionally, the value of each domain and its orientation was quantified. While the 2+2 was about 1,000-fold more potent than the IgG-Het (or 1+1T), it was only 6-fold more potent than the 2+1, and 20-25 fold more potent than the 1+2 or 1+1C. These data demonstrate that the second scFv imparts about 6-fold change in activity (2+2 is 6-fold better than 2+1), the bivalent Fab imparts about 25-fold change (2+2 is up to 25-fold better than 1+2 domain) and the Cis/Trans orientation imparts another 50-fold change (1+1C is 50-fold better than 1+1T).

These results demonstrate that the HDTVS antibodies disclosed herein are useful in methods for treating a disease or condition, such as cancer.

### Example 7: Modifications of the 2+2 IgG-[L]-scFv and their Relative Immune Cell Activation

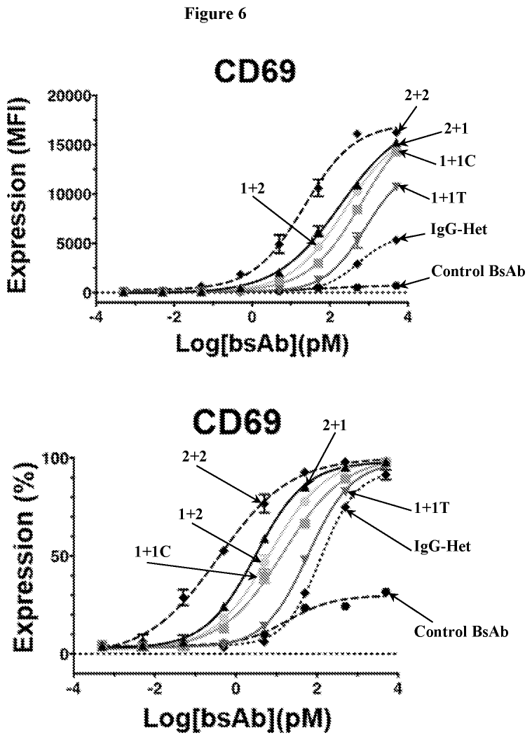

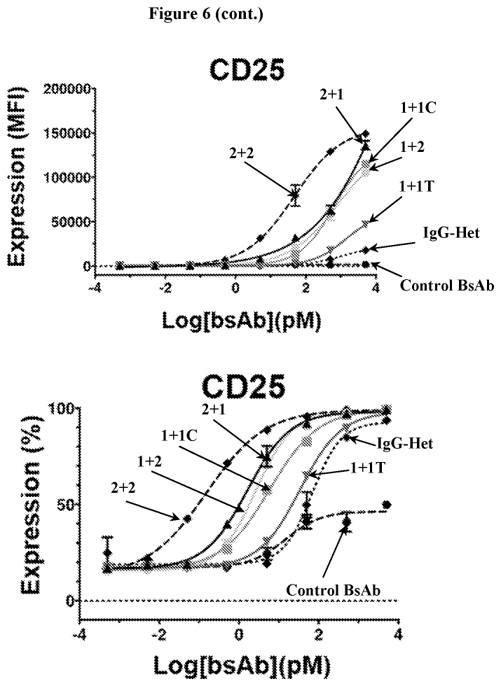

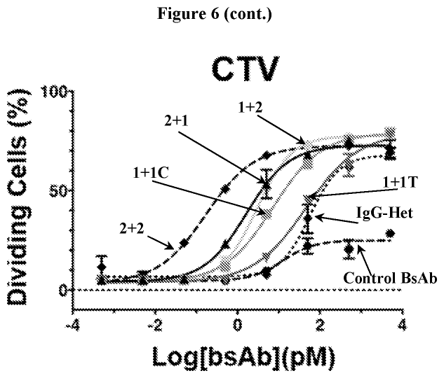

FIG. 6 describes the cell activation properties of each IgG-[L]-scFv variant in vitro. As illustrated in FIG. 6, the variations made to the IgG-[L]-scFv variants significantly influence their capacity to activate immune cells. The upper panels show upregulation of CD69 expression on T cells after 24 hours of in vitro coculture with varying concentrations of each BsAb and GD2(+) M14Luc tumor cells. As in FIG. 5, valency and cis/trans orientation appear to play an important role, suggesting that the activation potency and cytotoxicity are correlated. The 2+2 BsAb again displayed its superiority over all other variants tested, at both the level of expression level of CD69 (left) and the frequency of CD69(+) cells (right). Removal of a single domain (2+1 or 1+2) markedly lowered activation, and was made worse with the transition to 1+1C, 1+1T and finally IgG-Het. A similar pattern emerged after 96 hr of coculture (bottom panel). CD25 expression remained the highest for the 2+2, both in terms of expression level (left) and frequency of CD25(+) (center) cells. All other variants showed reduced activation of effector T cells. Proliferation was also measured using Cell Trace Violet (CTV) dilution. T cells were labeled with the cell penetrating dye CTV and incubated with target cells (M14Luc) and titrated with BsAb for 96 hrs. The frequency of cells fluorescing with less remaining CTV than an unstimulated control was considered to have divided at least once. As such, proliferation was the greatest for the 2+2 and reduced for all other IgG-[L]-scFv variants (right). No activation or proliferation was observed with any construct in the absence of tumor cells (data not shown) indicating that there is minimal activation without target antigen. These results demonstrate that a cis interaction is considerably more potent than a trans interaction (1+1C vs 1+1T) and furthermore that two cis interactions are more potent than one (2+2 vs 1+1C or 1+2 or 2+1) (two cis interactions are only possible in a dual bivalent approach, such as the 2+2 IgG-[L]-scFv).

These results demonstrate that the HDTVS antibodies disclosed herein are useful in methods for treating a disease or condition, such as cancer.

### Example 8: Modifications of the 2+2 IgG-[L]-scFv and their Relative In Vivo Tumor Clearance

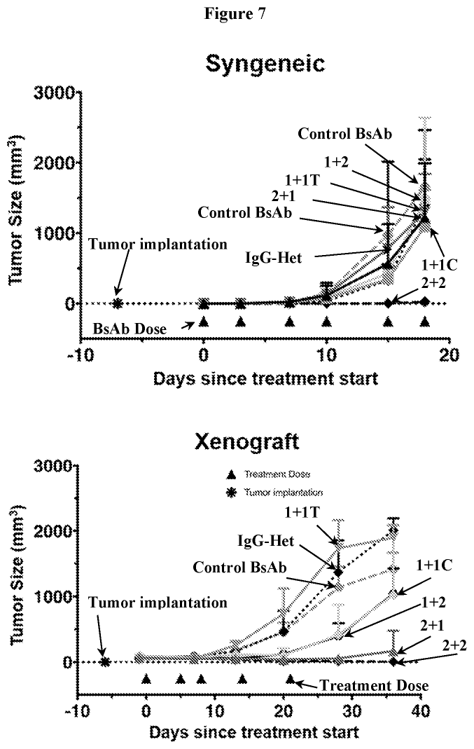

FIG. 7 describes the in vivo anti-tumor activity of each IgG-[L]-scFv variant in two different tumor models. As illustrated in FIG. 7, the in vivo anti-tumor activity of each variant largely correlated with in vitro cytotoxicity. In the xenograft model (right) the strongest anti-tumor activity was imparted by the 2+2 BsAb. Surprisingly, the 2+1 was very similar, with only a slight difference in tumor recurrence (5/5 CR for both). As with the cytotoxicity data, the next most effective were the 1+1C and 1+2, validating both in vitro findings that the cis orientation is superior to the trans and the 2+1 was superior to the 1+2. All other variants (1+1T, IgG-Het, control BsAb) failed to show any effect on tumor growth. In the more aggressive syngeneic model using EL.4 tumors (as done in FIG. 1), no IgG-[L]-scFv variant aside from the 2+2 showed an anti-tumor effect. As opposed to the xenograft model where activated T-cells are directly administered to the mouse, the syngeneic model requires activation in situ, suggesting that the in vitro cell activation differences may manifest in vivo leading to diminished capacity to shrink tumors. Taken together, these results suggest that the optimal BsAb platform is capable of strong cell activation in the presence of antigen, and that bivalency toward both cell populations, target cells and effector cells, is critical. In addition, these results confirm the importance of two cis-interactions in a bispecific antibody (2+2) over all single cis-interacting variants (2+1, 1+1C, 1+2) or non-cis interacting variants (1+1T, 1+1H).

These results demonstrate that the HDTVS antibodies disclosed herein are useful in methods for treating a disease or condition, such as cancer.

### Example 9: 2+2 IgG-[L]-scFv is Superior to Other Bivalent Antibody Designs

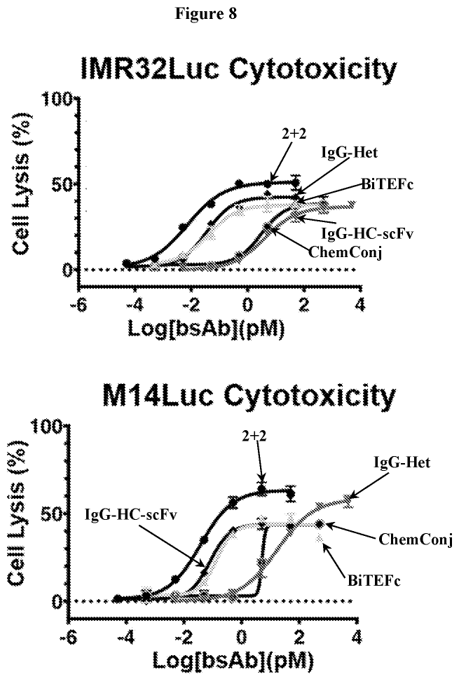

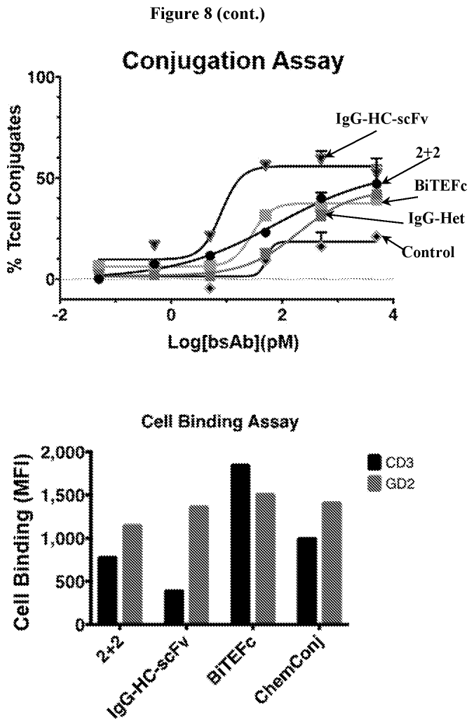

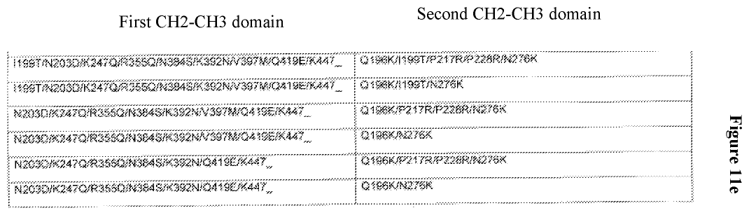

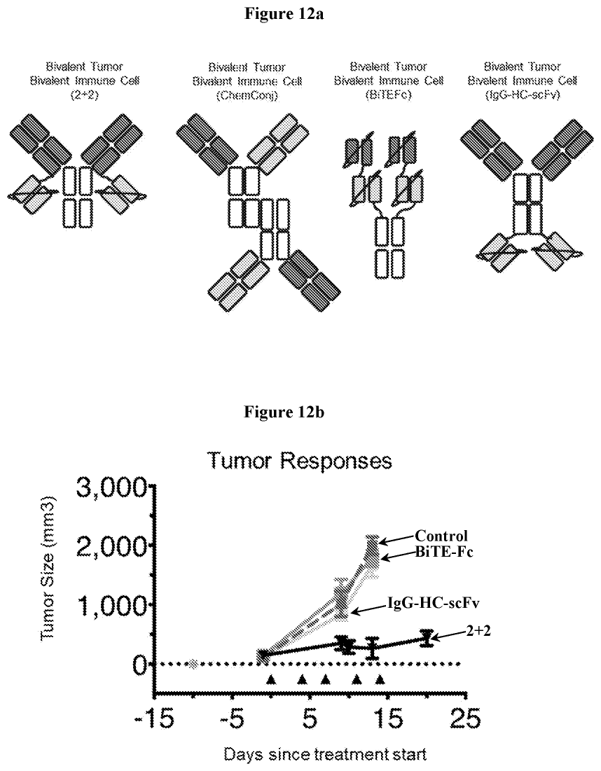

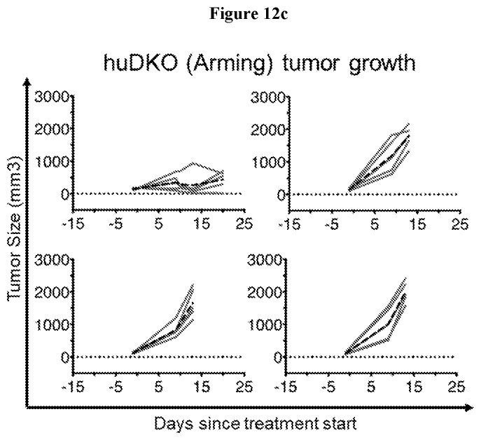

FIG. 8 shows cytotoxicity and conjugate formation activity from 3 additional 2+2 designs, thus demonstrating the overall superiority of the IgG-[L]-scFv format. The 2+2 IgG-[L]-scFv format was more demonstrably more potent than other conventional 2+2 formats. The IgG-chemical conjugate (Yankelevich et al., Pediatr Blood Cancer 59:1198-1205 (2012)) the IgG-[H]-scFv (with scFv attached at the C-terminus of the HC instead of the LC of the IgG; Coloma & Morrison, Nat Biotechnol 15:159-163 (1997)) and the BITE-Fc, all failed to kill cells as potently in vitro, compared with the IgG-[L]-scFv design. The poor cytotoxic effects were observed despite apparently improved conjugate formation activity (bottom left) and cell binding activity (bottom right). These results demonstrate that the structural features of the IgG-[L]-scFv format (unique flexibility, orientations and arrangements of the four antigen binding domains) may be correlated with effects on T-cell recruitment, activation and cytotoxicity. FIGS. 12a-12c show the in vivo anti-tumor activity from two additional 2+2 designs, thus confirming the overall superiority of the IgG-[L]-scFv format (2+2). Using an in vivo T-cell arming model, only the IgG-[L]-scFv format (2+2) of the present technology was able to inhibit tumor growth. Strikingly, despite the dual bivalency of the dimeric BiTE-Fc and the IgG-[H]-scFv, both failed to display any anti-tumor activity compared to the control BsAb. These results confirm the in vitro findings, that the superiority of the IgG-[L]-scFv design is not strictly due to decreased distance between binding domains, but instead suggests that the potency of the IgG-[L]-scFv is not simply a function of minimization of intermembrane distance. Rather, the exceptional in vitro and in vivo potency of the IgG-[L]-scFv may be attributed at least in part to the properties of cis-configured Fab and scFv domains, spaced apart with a single Ig domain (CL), such as stiffness or flexibility.

These results demonstrate that the HDTVS antibodies disclosed herein are useful in methods for treating a disease or condition, such as cancer.

### Example 10: 2+2 IgG-[L]-scFv and Subset of Variants Against Alternative Antigens

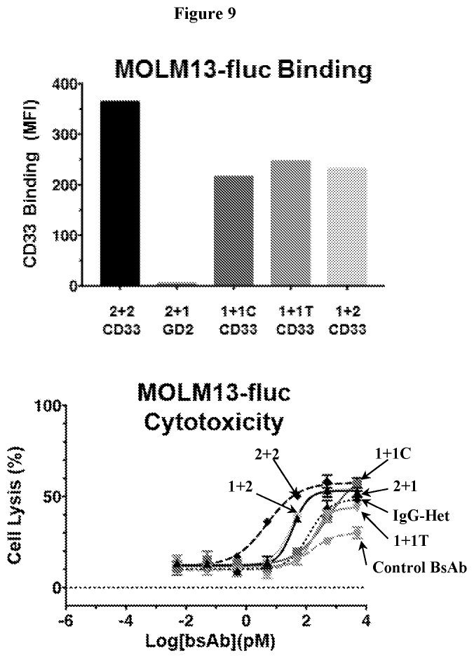

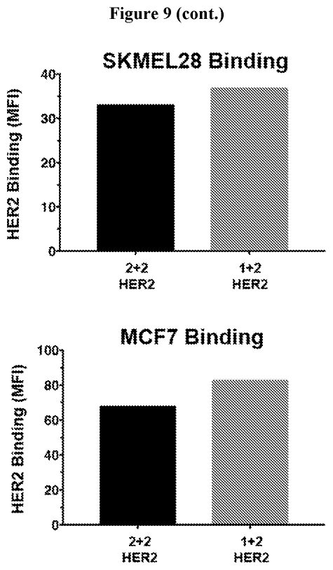

FIG. 9 describes some of the differences in activity observed with different tumor antigens. As illustrated in FIG. 9, the IgG-[L]-scFv platform does depend in part on the tumor antigen. When targeted to CD33 (top panels) a similar pattern of cell binding and cytotoxicity was found. CD33(+) MOLM13-fluc cells were assayed as described in FIG. 4 (left). As with GD2, reduction in valency (1+1T, 1+1C, or 1+2) significantly decreased binding activity. In terms of cytotoxicity, the Cis/Trans orientation appeared to play less of a role (both 1+1T and 1+C are most inferior, and equivalent to IgG-Het), and therefore the difference between the 2+1 and 1+2 was diminished. The lack of cis/trans difference may also explain the overall worse EC₅₀ against CD33(+) MOLM-13fluc as compared to GD2(+) M14Luc or IMR32Luc. When the tumor antigen was changed to HER2 (lower panels), and the antigen binding domains possessed significantly higher binding affinity, a different pattern was observed. 2+2 and 1+2 variants appeared identical, with similar tumor binding levels despite the monovalency. This suggests that with sufficiently high affinities, the second tumor binding domain is dispensable, as predicated in the Hi1+1+2 HDTVS design.

These results demonstrate that the HDTVS antibodies disclosed herein are useful in methods for treating a disease or condition, such as cancer.

### Example 11: Hi1+1+2 and Lo1+1+2 Proof of Concept Studies

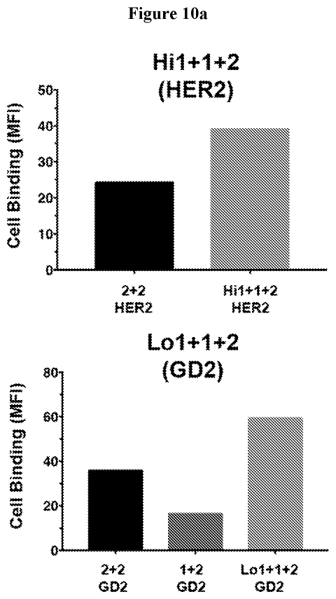

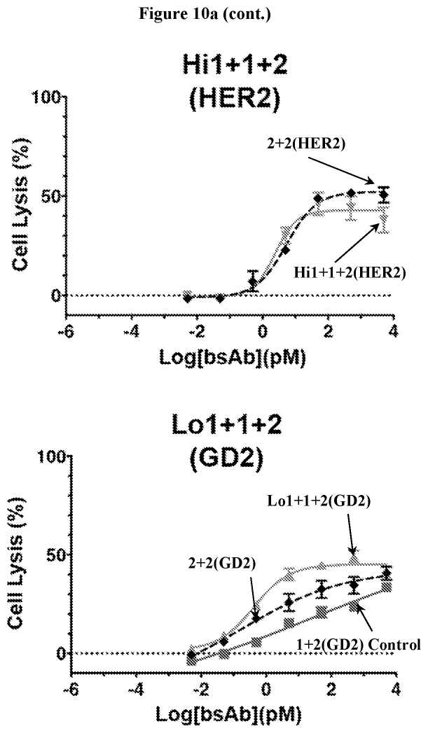

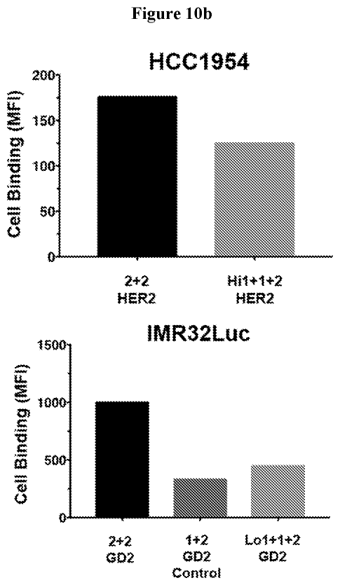

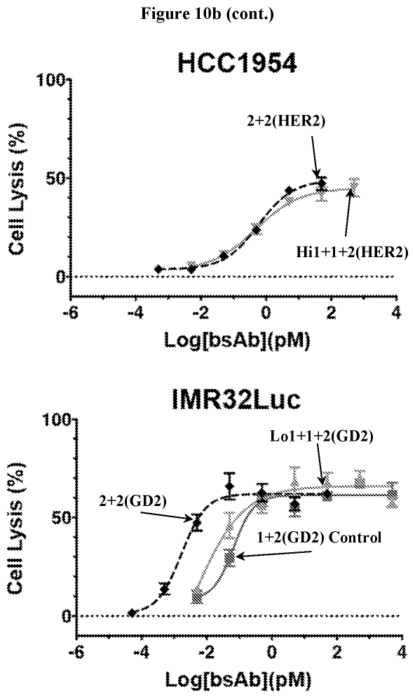

As depicted in FIG. 10a (left side), the 2(HER2)+2(CD3) functions similarly to the 1(HER2)+2(CD3), where only one Fab domain binds the tumor and the second Fab recognizes an irrelevant antigen, due to the very high affinity interaction between HER2 and the anti-HER2 Fab used (Herceptin). In both FACS binding (top) and an in vitro cytotoxicity assay (bottom) with U2OS cells, the 2(HER2)+2(CD3) and the 1(HER2)+2(CD3) were indistinguishable, highlighting the possibility of using the second Fab arm to target a separate antigen. Conversely, the Lo1(GD2)+1(GD2)+2(CD3) (right side), shows the utility of two separate tumor antigen specificities when binding affinities are sufficiently low. Here the 2(GD2)+2(CD3), the 1(GD2)+2(CD3) and Lo1(GD2)+1(GD2)+2(CD3) showed major differences that are explained by the differences in valency between constructs. In both FACS binding (top) and in vitro cytotoxicity (bottom) with U2OS cells, the 2(GD2)+2(CD3) displayed superior activity over a 1(GD2)+2(CD3) format having an irrelevant second specificity (thus limiting binding to monovalency). However, adding a second relevant Fab binding specificity (e.g. HER2) in Lo1(GD2)+1(HER2)+2(CD3) was able to rescue this defect and even improve binding and killing. These results highlight the utility of targeting two separate antigens on the same cell when the Fab affinity for each individual antigen is sufficiently low (e.g., 100 pM to 100 nM K_{D}). Additionally, the approximately 100-fold difference in EC₅₀ between the Lo1(GD2)+1(HER2)+2(CD3) and 1(GD2)+2(CD3) validates the improved therapeutic index between monovalent and bivalent binding of a Lo1(GD2)+1(HER2)+2(CD3) construct. Had the second specificity (i.e. HER2) of the Lo1+1+2(GD2) been irrelevant (no binding to tumor or T cells), it would have functioned as the 1(GD2)+2(CD3) with 100-fold less activity. This is in contrast to the 2+2 which would not be able to distinguish a dual-antigen positive tumor from a GD2(+) normal tissue (such as peripheral nerves).

As shown in FIG. 10b, when these two sets of constructs were presented to tumor cells expressing high levels of only one antigen (HER2 and GD2, left and right sides respectively), the same patterns were observed. With the 2(HER2)+2(CD3) and 1(HER2)+2(CD3), similar FACS binding and cytotoxicity were observed against the HCC1954 cell line which shows high expression of HER2(+). However, stronger binding and cytotoxicity was observed with the 2(GD2)+2(CD3) compared to the 1(GD2)+2(CD3) and a Lo1(GD2)+1(HER2)+2(CD3) having an irrelevant second specificity (second Fab domain did not recognize the tumor cell line IMR32Luc).

Taken together, with a sufficiently high effective affinity interaction a 1+2 IgG-[L]-scFv functions identically to a 2+2, suggesting the Hi1+1+2 can be used to target two separate antigens instead of just one. However, with a sufficiently low effective affinity interaction, a Lo1+1+2 can provide an improved therapeutic index to distinguish between single antigen positive normal tissue and double antigen positive tumor cells.

These results demonstrate that the HDTVS antibodies disclosed herein are useful in methods for treating a disease or condition, such as cancer.

### Example 12: Binding Affinity and Cytotoxic Selectivity of the Low Affinity 1+1+2 Format Antibodies of the Present Technology

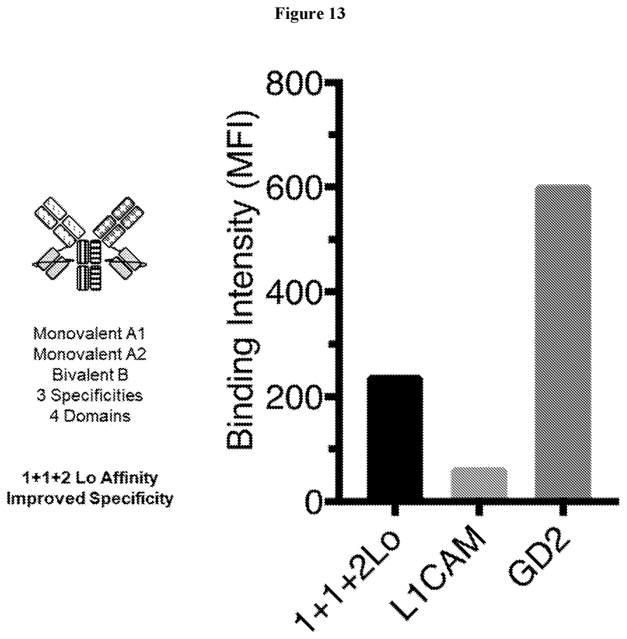

The binding affinity of L1CAM/GD2 1+1+2 Lo, a heterodimeric 1+1+2Lo format antibody, which can bind ganglioside GD2 and adhesion protein L1CAM simultaneously, was compared with homodimeric formats against GD2 and L1CAM. Neuroblastoma cells (IMR32) were incubated with each antibody for 30 minutes at 4° C., washed and incubated with a fluorescent anti-human secondary antibody. After the final wash, the cells were analyzed using flow cytometry. As shown in FIG. 13, the binding of the low affinity 1+1+2 HDTVS antibody was stronger than that of the anti-L1CAM homodimeric antibody, but weaker than the anti-GD2 homodimeric antibody, thus showing improved targeting specificity for tumors expressing both GD2 and L1CAM.

The combined binding effect of GD2/B7H3 1+1+2 Lo, a heterodimeric 1+1+2Lo format antibody, which can bind both GD2 and B7H3 simultaneously was also compared with the homodimeric format antibodies against GD2 and B7H3, and monovalent control antibodies against GD2 or B7H3. Osteosarcoma cells (U2OS) were incubated with each antibody for 30 minutes at 4° C., washed and incubated with a fluorescent anti-human secondary antibody. After the final wash, the cells were analyzed using flow cytometry. As shown in FIG. 15, the binding of the low affinity 1+1+2 heterodimer antibody was similar to the anti-B7H3 homodimeric antibody, but weaker than the anti-GD2 homodimeric antibody. Importantly, the GD2/B7H3 1+1+2 Lo HDTVS antibody also shows improved binding over monovalent control antibodies, thus demonstrating cooperative binding of the heterodimeric GD2/B7H3 1+1+2 Lo antibody.

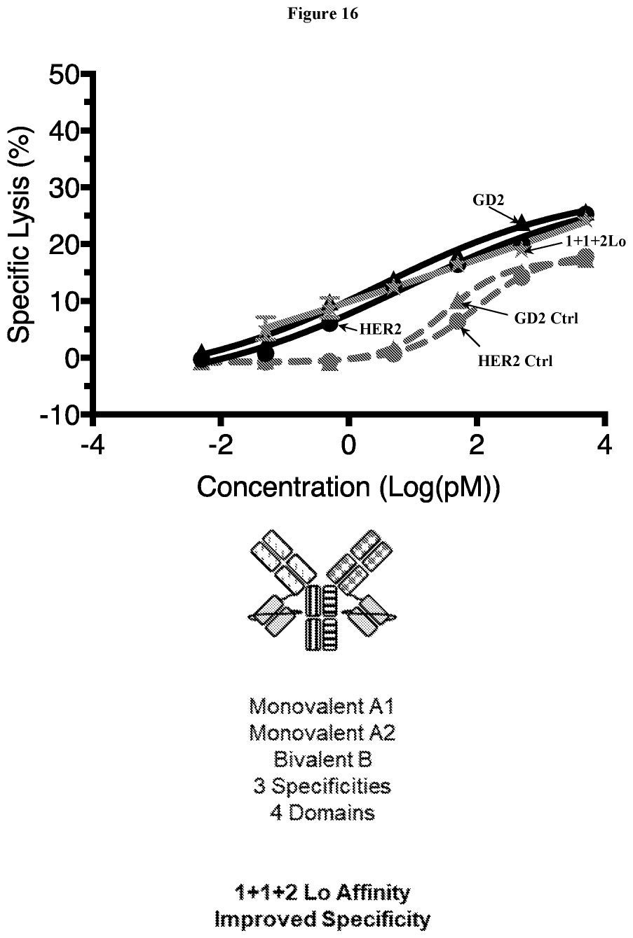

To assess the cytotoxic selectivity of the low affinity 1+1+2Lo format antibodies of the present technology, HER2/GD2 1+1+2 Lo, a heterodimeric 1+1+2Lo format antibody, which can bind both GD2 and HER2 simultaneously, was studied. In this format, a low affinity HER2 sequence was used. Homodimeric formats against GD2 and HER2, and monovalent control antibodies against GD2 or HER2 were included for reference. Osteosarcoma cells (U2OS) were first incubated with ⁵¹Cr for one hour. After the incubation, the ⁵¹Cr labeled target cells were mixed with serial dilutions of the antibodies and activated human T-cells for four hours at 37° C. After four hours, supernatant was harvested and analyzed on a gamma counter to quantify the released ⁵¹Cr. As shown in FIG. 16, the low affinity 1+1+2 heterodimer antibody killed U2OS cells as effectively as the anti-GD2 and anti-HER2 homodimeric antibodies and showed clear superiority over the monovalent control formats. Therefore, the 1+1+2Lo design exhibited 10-100-fold lower cytotoxic potency in cells expressing each individual antigen compared to target cells expressing both antigens simultaneously. A homodimeric design for either GD2 or HER2 would not be expected to exhibit such selectivity.

These results demonstrate the selective cytotoxicity could be attained with the 1+1+2Lo design by targeting cells expressing each individual antigen with 10-100-fold lower cytotoxic potency than targets expressing both antigens simultaneously.

Accordingly, the 1+1+2Lo format antibodies of the present technology are useful in methods for treating a disease or condition, such as cancer.

### Example 13: Binding Affinity and Cytotoxic Dual Specificity of the 1+1+2Hi Format Antibodies of the Present Technology

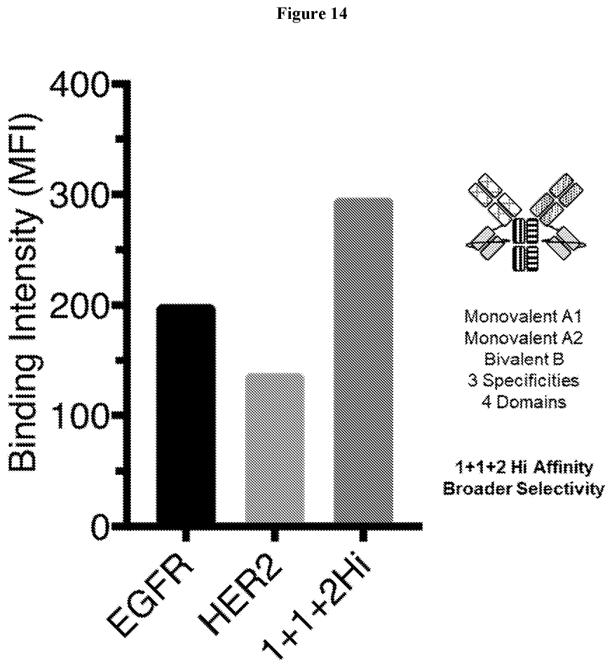

To assess the binding affinity of the heterodimeric 1+1+2Hi format antibodies of the present technology, the combined binding effect of HER2/EGFR 1+1+2Hi, a heterodimeric 1+1+2Hi format antibody, which can bind both HER2 and EGFR, either simultaneously or separately, was analyzed. Homodimeric formats against HER2 and EGFR were included for reference. Desmoplastic Small Cell Round Tumor cells (JN-DSRCT1) were incubated with each antibody for 30 minutes at 4° C., washed and incubated with a fluorescent anti-human secondary antibody. As shown in FIG. 14, the binding of the high affinity 1+1+2 heterodimer antibody was stronger than that of either anti-HER2 or anti-EGFR homodimeric antibodies, while maintaining specificity for both antigens, thus demonstrating cooperative binding.

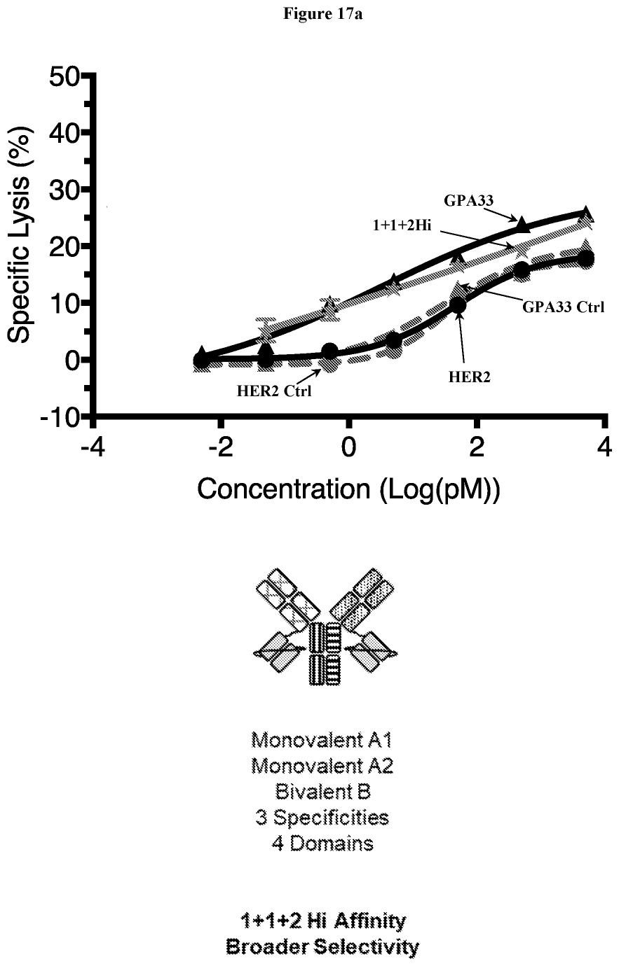

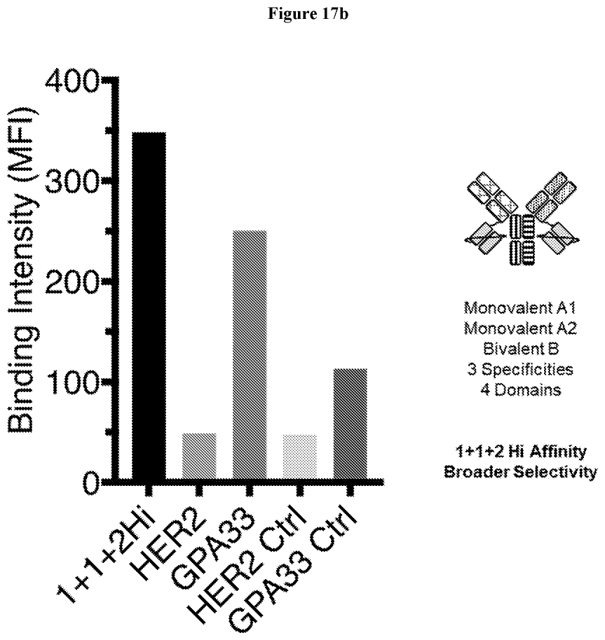

HER2/GPA33 1+1+2 Hi, a heterodimeric 1+1+2Hi format antibody, which can bind both GPA33 and HER2 either simultaneously or separately, was compared with the homodimeric format antibodies against GPA33 and HER2, and monovalent control antibodies against GPA33 or HER2. To compare the combined binding effect, colon cancer cells (Colo205) were incubated with each antibody for 30 minutes at 4° C., washed and incubated with a fluorescent anti-human secondary antibody. After the final wash, the cells were analyzed using flow cytometry. HER2/GPA33 1+1+2 Hi antibody bound both HER2 and GPA33 on Colo205 cells, either simultaneously or separately (FIG. 17b). As shown in FIG. 17b, the binding affinity of the 1+1+2Hi heterodimer antibody was stronger than either anti-HER2 or anti-GPA33 homodimeric and monovalent control antibodies, while maintaining specificity for both antigens, thus demonstrating cooperative binding.

To evaluate the cytotoxic specificity of the HER2/GPA33 1+1+2Hi format antibody, colon cancer cells (Colo205) were first incubated with ⁵¹Cr for one hour. After the incubation, the ⁵¹Cr labeled target cells were mixed with serial dilutions of the indicated antibody and activated human T-cells for four hours at 37° C. After four hours, the supernatant was harvested and read on a gamma counter to quantify the released ⁵¹Cr. Cytotoxicity was measured as the % of released ⁵¹Cr from maximum release. As shown in FIG. 17a, the high affinity 1+1+2 heterodimer antibody killed Colo205 cells as effectively as the anti-GPA33 homodimeric antibody, but with greater potency than the anti-HER2 homodimeric antibody and monovalent control antibodies. These results demonstrate functional cooperativity between the HER2 and GPA33 antigen binding domains, and illustrate that the dual specificity of a 1+1+2Hi format does not significantly compromise its cytotoxicity against either antigen individually.

Accordingly, the 1+1+2Hi format antibodies of the present technology are useful in methods for treating a disease or condition, such as cancer.

### Example 14: Combined Binding Effects and Cytokine Release Induced by the 2+1+1 Format Antibodies of the Present Technology

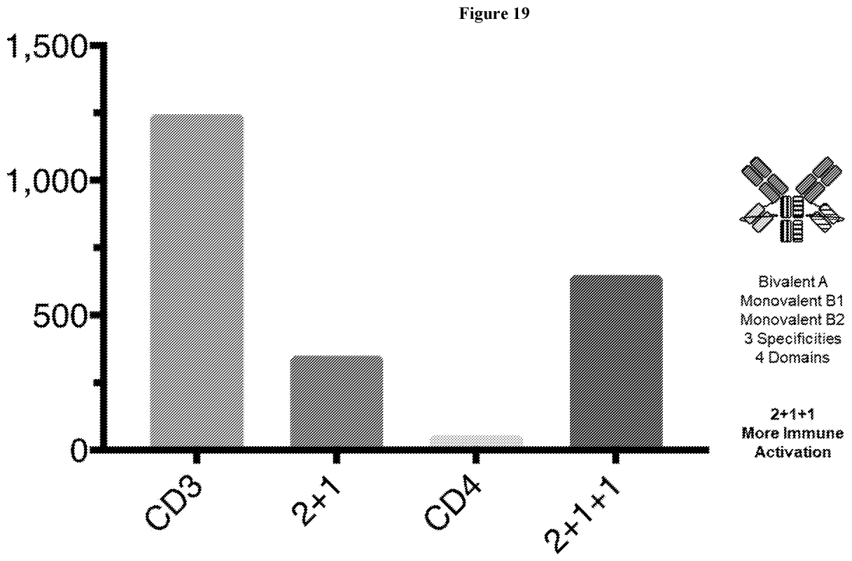

To evaluate the combined binding effects of the heterodimeric 2+1+1 format antibodies of the present technology, several heterodimeric 2+1+1 format antibodies were compared with their corresponding homodimeric format antibodies and monovalent control antibodies. For example, CD3/CD4 2+1+1, a heterodimeric 2+1+1 format antibody that can bind both CD3 and CD4 simultaneously was compared with its corresponding bivalent format antibodies against CD3 and CD4, and a monomeric CD3 binder (2+1). For this binding assay, active human T cells were incubated with each antibody for 30 minutes at 4° C., washed and incubated with a fluorescent anti-human secondary antibody. After the final wash, the cells were analyzed using flow cytometry. As shown in FIG. 19, the binding of CD3/CD4 2+1+1 antibodies showed enhanced binding compared to the bivalent CD4 antibody and monomeric CD3 binder (2+1), thus demonstrating cooperative binding.

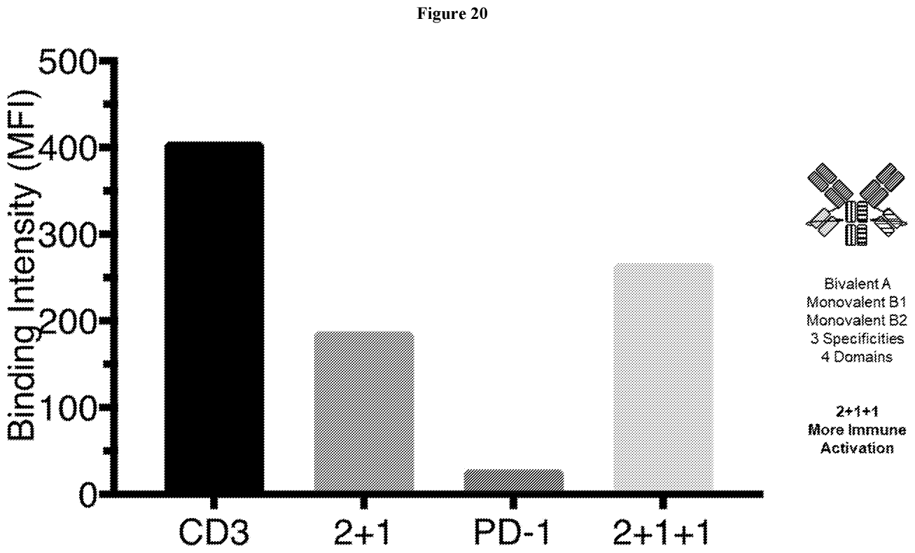

Similarly, binding of CD3/PD-1 2+1+1, a heterodimeric 2+1+1 format antibody that can bind both CD3 and PD-1 simultaneously, was compared with homodimeric anti-PD-1 and anti-CD3 antibodies, and with an anti-CD3 monomeric (2+1) binder. For this binding assay active human T cells were incubated with each antibody for 30 minutes at 4° C., washed and incubated with a fluorescent anti-human secondary antibody. After the final wash, the cells were analyzed using flow cytometry. As shown in FIG. 20, the 2+1+1 heterodimer antibody bound cells better than either anti-PD-1 homodimeric antibody or anti-CD3 monomeric (2+1) binder, thus demonstrating cooperative binding. Collectively, these data demonstrate that a heterodimeric 2+1+1 format antibody of the present technology binds its target better than the corresponding weaker-binding homodimeric antibody and its corresponding monomeric (2+1) binder, thus demonstrating cooperative binding.

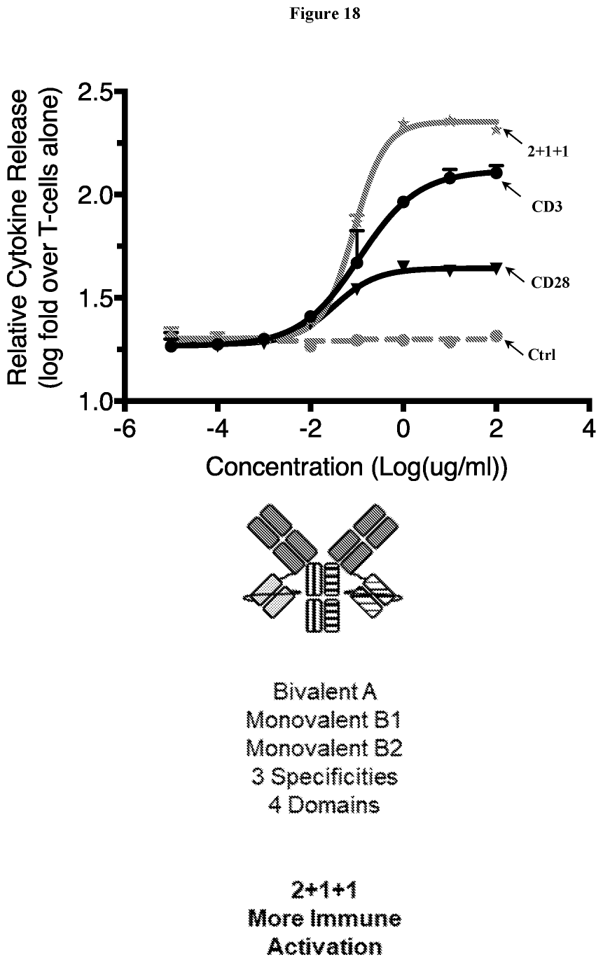

Next, cytokine release induced by CD3/CD28 2+1+1, a heterodimeric 2+1+1 format antibody, was analyzed. The homodimeric format antibodies against CD3 and CD28 were included for reference. Naïve human T-cells and melanoma tumor cells (M14) were co-cultured along with the indicated BsAb for 20 hours. Culture supernatants were harvested following the incubation and analyzed for secreted cytokine IL-2 by FACS. Data were normalized to T-cell cytokine release after 20 hours without target cells or antibody. As shown in FIG. 18, the CD3/CD28 2+1+1 antibody showed more potent cytokine release activity compared to either CD3 or CD28 engagement alone, illustrating cooperative activity from dual CD3/CD28 engagement. These results demonstrate the utility of a heterodimeric 2+1+1 design that can bind both CD3 and CD28 on T-cells.

Accordingly, the 2+1+1 format antibodies of the present technology are useful in methods for treating a disease or condition, such as cancer.

### Example 15: Comparison of the IgG-L-scFv Format of the Present Technology with BiTE-Fc and IgG-H-scFv Formats

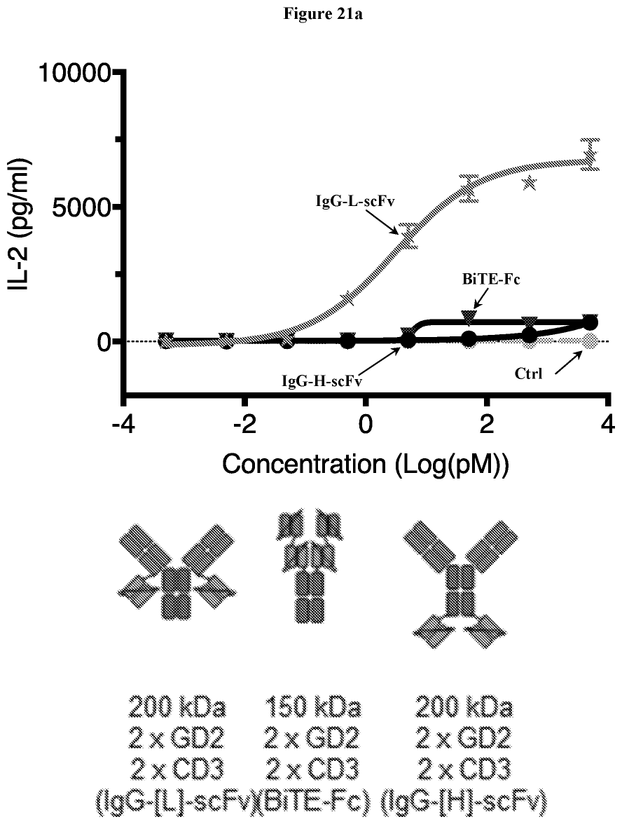

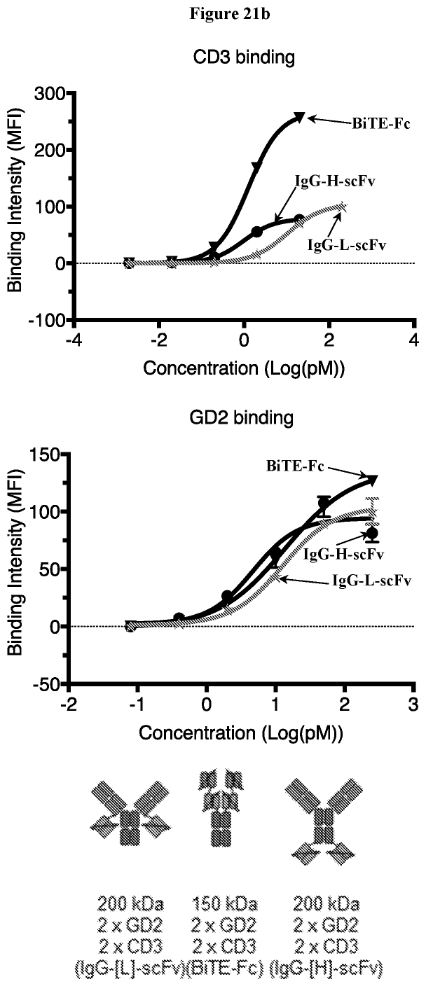

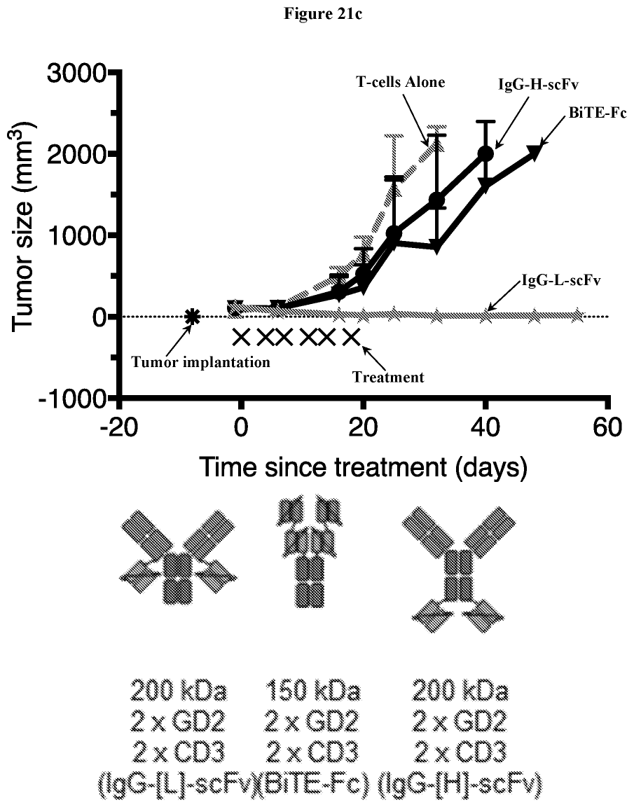

The IgG-L-scFv design was next compared with two other common dual bivalent design strategies: the BiTE-Fc and the IgG-H-scFv formats. First, to compare cytokine release induced by IgG-L-scFv design compared to BiTE-Fc and the IgG-H-scFv, naïve T-cells and melanoma tumor cells (M14) were co-cultured along with each BsAb for 20 hours. Culture supernatants were harvested and analyzed for secreted cytokine IL-2. Data were normalized to T-cell cytokine release after 20 hours without target cells or antibody. As shown in FIG. 21a, the IgG-L-scFv design (2+2) exhibited unusually potent T-cell functional activity compared to other dual bivalent T-cell bispecific antibody formats.

To compare binding intensity, T-cells and melanoma tumor cells (M14) were separately incubated with each antibody for 30 minutes at 4° C., washed and incubated with a fluorescent anti-human secondary antibody. After the final wash, the cells were analyzed using flow cytometry. As shown in FIG. 21b (upper panel), IgG-L-scFv design showed unusually weak T-cell binding activity compared to other dual bivalent T-cell bispecific antibody formats. In contrast to their GD2 binding activity (FIG. 21b (middle panel)), each BsAb demonstrated quite different T-cell binding activities. These data demonstrated how the IgG-L-scFv design is uniquely different than other dual-bivalent designs, with each scFv showing incomplete bivalent binding. Although the inclusion of two scFv domains in the IgG-L-scFv did result in an improvement over monovalent designs, it still did not compare to the binding activity of the 2+2 IgG-H-scFv or 2+2 BiTE-Fc designs, illustrating the sterically hindered binding of this format.

The effect of the observed binding and cytokine release profiles on the in vivo antitumor activity was explored next. Immunodeficient mice (Balb/c IL-2Rgc−/−, Rag2−/−) were implanted with neuroblastoma cells (IMR32) subcutaneously and treated with intravenous activated T-cells and antibody (2-times per week). Tumors sizes were measured by caliper. As shown in FIG. 21c, the IgG-L-scFv design antibodies inhibited tumor growth. In comparison, the IgG-H-scFv and BiTE-Fc design antibodies showed a borderline in vivo effect. Therefore, in contrast to the IgG-H-scFv (2+2HC) and the BiTE-Fc (2+2B) designs, the IgG-L-scFv format (2+2) demonstrated significant cytokine IL-2 responses in vitro (FIG. 21a), which correlated with stronger in vivo activity (FIG. 21c).

Collectively, these data demonstrate the in vivo superiority of the IgG-L-scFv format antibodies in that only the IgG-L-scFv format antibodies were capable of inhibiting tumor growth in animals in contrast to other dual bivalent designs.

These results demonstrate that the HDTVS antibodies disclosed herein are useful in methods for treating a disease or condition, such as cancer.

### Example 16: Importance of Cis-Oriented Binding Domains with Respect to In Vitro Properties of an Anti-IgG-[L]-scFv Antibody

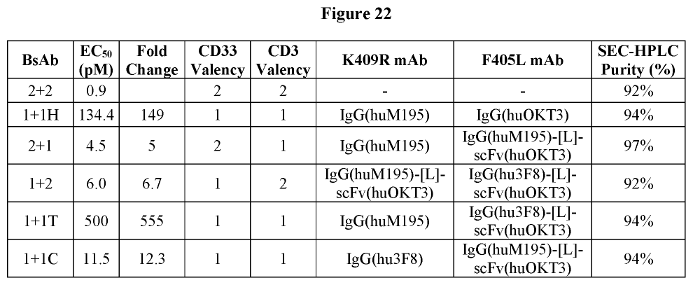

To further understand the in vitro properties of antibodies of various designs, a anti-CD33 IgG-[L]-scFv panel was created, and the in vitro cytotoxicity EC₅₀, fold-difference in EC₅₀, antigen valency, heterodimer design and protein purity were examined. FIG. 22 summarizes the data. Fold change was based on the EC₅₀ of 2+2. Purity was calculated as the fraction of protein at correct elution time out of the total protein by area under the curve of the SEC-HPLC chromatogram. For the cytotoxicity assays, CD33-transfected cells (Nalm6) were first incubated with ⁵¹Cr for one hour. Afterwards, ⁵¹Cr labeled target cells were mixed with serial titrations of the indicated antibody and activated human T-cells for four hours at 37° C. The supernatant was harvested and analyzed on a gamma counter to quantify the released ⁵¹Cr. Cytotoxicity was measured as the % of released ⁵¹Cr from maximum release. These results shown in FIG. 22 confirm the relative importance of cis-oriented binding domains in an additional antigen system (CD33) which is much more membrane distal than GD2 (see FIG. 5).

These results demonstrate that the HDTVS antibodies disclosed herein are useful in methods for treating a disease or condition, such as cancer.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein:
a. the first polypeptide chain comprises in the N-terminal to C-terminal direction:
i. a light chain variable domain of a first immunoglobulin (VL-1) that is capable of specifically binding to a first epitope;
ii. a light chain constant domain of the first immunoglobulin (CL-1);
iii. a flexible peptide linker comprising the amino acid sequence (GGGGS)₃ (SEQ ID NO: 2506); and
iv. a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ (SEQ ID NO: 2507) to form a single-chain variable fragment;
b. the second polypeptide comprises in the N-terminal to C-terminal direction:
i. a heavy chain variable domain of the first immunoglobulin (VH-1) that is capable of specifically binding to the first epitope;
ii. a first CH1 domain of the first immunoglobulin (CH1-1); and
iii. a first heterodimerization domain of the first immunoglobulin, wherein the first heterodimerization domain is incapable of forming a stable homodimer with another first heterodimerization domain;
c. the third polypeptide comprises in the N-terminal to C-terminal direction:
i. a heavy chain variable domain of a third immunoglobulin (VH-3) that is capable of specifically binding to a third epitope;
ii. a second CH1 domain of the third immunoglobulin (CH1-3); and
iii. a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin;
d. the fourth polypeptide comprises in the N-terminal to C-terminal direction:
i. a light chain variable domain of the third immunoglobulin (VL-3) that is capable of specifically binding to the third epitope;
ii. a light chain constant domain of the third immunoglobulin (CL-3);
iii. a flexible peptide linker comprising the amino acid sequence (GGGGS)₃ (SEQ ID NO: 2506); and
iv. a light chain variable domain of a fourth immunoglobulin (VL-4) that is linked to a complementary heavy chain variable domain of the fourth immunoglobulin (VH-4), or a heavy chain variable domain of a fourth immunoglobulin (VH-4) that is linked to a complementary light chain variable domain of the fourth immunoglobulin (VL-4), wherein VL-4 and VH-4 are capable of specifically binding to the second epitope, and are linked together via a flexible peptide linker comprising the amino acid sequence (GGGGS)₆ (SEQ ID NO: 2507) to form a single-chain variable fragment, and
wherein each of VL-1 and VL-3 independently comprises the CDR1 sequence, the CDR2 sequence and the CDR3 sequence of a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345; and/or
wherein each of VH-1 and VH-3 independently comprises the CDR1 sequence, the CDR2 sequence and the CDR3 sequence of a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349.

2. A heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein:
a. the first polypeptide chain comprises in the N-terminal to C-terminal direction:
i. a light chain variable domain of a first immunoglobulin (VL-1) that is capable of specifically binding to a first epitope;
ii. a light chain constant domain of the first immunoglobulin (CL-1);
iii. a flexible peptide linker comprising the amino acid sequence (GGGGS)₃ (SEQ ID NO: 2506); and
iv. a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein the VL-2 and VH-2 are capable of specifically binding to a second epitope, and are linked together via a flexible peptide linker comprising the amino acid sequence (GGGGS)₆ (SEQ ID NO: 2507) to form a single-chain variable fragment;
b. the second polypeptide comprises in the N-terminal to C-terminal direction:
i. a heavy chain variable domain of the first immunoglobulin (VH-1) that is capable of specifically binding to the first epitope;
ii. a first CH1 domain of the first immunoglobulin (CH1-1); and
iii. a first heterodimerization domain of the first immunoglobulin, wherein the first heterodimerization domain is incapable of forming a stable homodimer with another first heterodimerization domain;
c. the third polypeptide comprises in the N-terminal to C-terminal direction:
i. a heavy chain variable domain of a third immunoglobulin (VH-3) that is capable of specifically binding to the first epitope;
ii. a second CH1 domain of the third immunoglobulin (CH1-3); and
iii. a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin;
d. the fourth polypeptide comprises in the N-terminal to C-terminal direction:
i. a light chain variable domain of the third immunoglobulin (VL-3) that is capable of specifically binding to the first epitope;
ii. a light chain constant domain of the third immunoglobulin (CL-3);
iii. a flexible peptide linker comprising the amino acid sequence (GGGGS)₃ (SEQ ID NO: 2506); and
iv. a light chain variable domain of a fourth immunoglobulin (VL-4) that is linked to a complementary heavy chain variable domain of the fourth immunoglobulin (VH-4), or a heavy chain variable domain of a fourth immunoglobulin (VH-4) that is linked to a complementary light chain variable domain of the fourth immunoglobulin (VL-4), wherein the VL-4 and VH-4 are capable of specifically binding to a third epitope, and are linked together via a flexible peptide linker comprising the amino acid sequence (GGGGS)₆ (SEQ ID NO: 2507) to form a single-chain variable fragment, and
wherein each of VL-2 and VL-4 independently comprises the CDR1 sequence, the CDR2 sequence and the CDR3 sequence of a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345; and/or
wherein each of V-2 and VH-4 independently comprises the CDR1 sequence, the CDR2 sequence and the CDR3 sequence of a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349.

3. A heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein:
a. the first polypeptide chain comprises in the N-terminal to C-terminal direction:
i. a light chain variable domain of a first immunoglobulin (VL-1) that is capable of specifically binding to a first epitope;
ii. a light chain constant domain of the first immunoglobulin (CL-1);
iii. a flexible peptide linker comprising the amino acid sequence (GGGGS)₃ (SEQ ID NO: 2506); and
iv. a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ (SEQ ID NO: 2507) to form a single-chain variable fragment;
b. the second polypeptide comprises in the N-terminal to C-terminal direction:
i. a heavy chain variable domain of the first immunoglobulin (VH-1) that is capable of specifically binding to the first epitope;
ii. a first CH1 domain of the first immunoglobulin (CH1-1); and
iii. a first heterodimerization domain of the first immunoglobulin, wherein the first heterodimerization domain is incapable of forming a stable homodimer with another first heterodimerization domain;
c. the third polypeptide comprises in the N-terminal to C-terminal direction:
i. a heavy chain variable domain of a third immunoglobulin (VH-3) that is capable of specifically binding to a third epitope;
ii. a second CH1 domain of the third immunoglobulin (CH1-3); and
iii. a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin;
d. the fourth polypeptide comprises in the N-terminal to C-terminal direction:
i. a light chain variable domain of the third immunoglobulin (VL-3) that is capable of specifically binding to the third epitope;
ii. a light chain constant domain of the third immunoglobulin (CL-3);
iii. a flexible peptide linker comprising the amino acid sequence (GGGGS)₃ (SEQ ID NO: 2506); and
iv. a light chain variable domain of a fourth immunoglobulin (VL-4) that is linked to a complementary heavy chain variable domain of the fourth immunoglobulin (VH-4), or a heavy chain variable domain of a fourth immunoglobulin (VH-4) that is linked to a complementary light chain variable domain of the fourth immunoglobulin (VL-4), wherein VL-4 and VH-4 are capable of specifically binding to the fourth epitope, and are linked together via a flexible peptide linker comprising the amino acid sequence (GGGGS)₆ (SEQ ID NO: 2507) to form a single-chain variable fragment; and
wherein each of VL-1 and VL-3 independently comprises the CDR1 sequence, the CDR2 sequence and the CDR3 sequence of a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345; and/or
wherein each of VH-1 and VH-3 independently comprises the CDR1 sequence, the CDR2 sequence and the CDR3 sequence of a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349; and/or
wherein each of VL-2 and VL-4 independently comprises the CDR1 sequence, the CDR2 sequence and the CDR3 sequence of a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345; and/or
wherein each of V-2 and VH-4 independently comprises the CDR1 sequence, the CDR2 sequence and the CDR3 sequence of a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349.

4. A heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein:
a. the first polypeptide chain comprises in the N-terminal to C-terminal direction:
i. a light chain variable domain of a first immunoglobulin (VL-1) that is capable of specifically binding to a first epitope;
ii. a light chain constant domain of the first immunoglobulin (CL-1);
iii. a flexible peptide linker comprising the amino acid sequence (GGGGS)₃ (SEQ ID NO: 2506); and
iv. a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ (SEQ ID NO: 2507) to form a single-chain variable fragment;
b. the second polypeptide comprises in the N-terminal to C-terminal direction:
i. a heavy chain variable domain of the first immunoglobulin (VH-1) that is capable of specifically binding to the first epitope;
ii. a first CH1 domain of the first immunoglobulin (CH1-1); and
iii. a first heterodimerization domain of the first immunoglobulin, wherein the first heterodimerization domain is incapable of forming a stable homodimer with another first heterodimerization domain;
c. the third polypeptide comprises in the N-terminal to C-terminal direction:
i. a heavy chain variable domain of a third immunoglobulin (VH-3) that is capable of specifically binding to the first epitope;
ii. a second CH1 domain of the third immunoglobulin (CH1-3); and
iii. a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin;
d. the fourth polypeptide comprises in the N-terminal to C-terminal direction:
i. a light chain variable domain of the third immunoglobulin (VL-3) that is capable of specifically binding to the first epitope; and
ii. a light chain constant domain of the third immunoglobulin (CL-3); and wherein VL-2 comprises the CDR1 sequence, the CDR2 sequence and the CDR3 sequence of a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345; and/or
wherein V-2 comprises the CDR1 sequence, the CDR2 sequence and the CDR3 sequence of a VH amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349, optionally wherein both VH-1 and VH-3 comprise the CDR1 sequence, the CDR2 sequence and the CDR3 sequence of a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349; and/or
wherein both VL-1 and VL-3 comprise the CDR1 sequence, the CDR2 sequence and the CDR3 sequence of a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345.

5. (canceled)

6. A heterodimeric multispecific antibody comprising a first polypeptide chain, a second polypeptide chain, a third polypeptide chain and a fourth polypeptide chain, wherein the first and second polypeptide chains are covalently bonded to one another, the second and third polypeptide chains are covalently bonded to one another, and the third and fourth polypeptide chain, and wherein:
a. the first polypeptide chain comprises in the N-terminal to C-terminal direction:
i. a light chain variable domain of a first immunoglobulin (VL-1) that is capable of specifically binding to a first epitope;
ii. a light chain constant domain of the first immunoglobulin (CL-1);
iii. a flexible peptide linker comprising the amino acid sequence (GGGGS)₃ (SEQ ID NO: 2506); and
iv. a light chain variable domain of a second immunoglobulin (VL-2) that is linked to a complementary heavy chain variable domain of the second immunoglobulin (VH-2), or a heavy chain variable domain of a second immunoglobulin (VH-2) that is linked to a complementary light chain variable domain of the second immunoglobulin (VL-2), wherein VL-2 and VH-2 are capable of specifically binding to a second epitope, and are linked together via a flexible peptide linker comprising the amino acid sequence (GGGGS)₆ (SEQ ID NO: 2507) to form a single-chain variable fragment;
b. the second polypeptide comprises in the N-terminal to C-terminal direction:
i. a heavy chain variable domain of the first immunoglobulin (VH-1) that is capable of specifically binding to the first epitope;
ii. a first CH1 domain of the first immunoglobulin (CH1-1); and
iii. a first heterodimerization domain of the first immunoglobulin, wherein the first heterodimerization domain is incapable of forming a stable homodimer with another first heterodimerization domain;
c. the third polypeptide comprises in the N-terminal to C-terminal direction:
i. a heavy chain variable domain of a third immunoglobulin (VH-3) that is capable of specifically binding to a third epitope;
ii. a second CH1 domain of the third immunoglobulin (CH1-3); and
iii. a second heterodimerization domain of the third immunoglobulin, wherein the second heterodimerization domain comprises an amino acid sequence or a nucleic acid sequence that is distinct from the first heterodimerization domain of the first immunoglobulin, wherein the second heterodimerization domain is incapable of forming a stable homodimer with another second heterodimerization domain, and wherein the second heterodimerization domain of the third immunoglobulin is configured to form a heterodimer with the first heterodimerization domain of the first immunoglobulin;
d. the fourth polypeptide comprises in the N-terminal to C-terminal direction:
i. a light chain variable domain of the third immunoglobulin (VL-3) that is capable of specifically binding to the third epitope; and
ii. a light chain constant domain of the third immunoglobulin (CL-3); and
wherein each of VL-1 and VL-3 independently comprises the CDR1 sequence, the CDR2 sequence and the CDR3 sequence of a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345; and/or
wherein each of VH-1 and VH-3 independently comprises the CDR1 sequence, the CDR2 sequence and the CDR3 sequence of a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349; and/or
wherein VL-2 comprises the CDR1 sequence, the CDR2 sequence and the CDR3 sequence of a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345; and/or
wherein V-2 comprises the CDR1 sequence, the CDR2 sequence and the CDR3 sequence of a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349.

7. The heterodimeric multispecific antibody of claim 1, wherein VH-1 or VH-3 comprise a V_{H} amino acid sequence selected from any one of SEQ ID NOs: 5, 13, 21, 29, 37, 45, 53, 61, 69, 77, 85, 93, 101, 109, 117, 125, 133, 149, 157, 165, 173, 181, 197, 205, 237, 245, 261, 277, 285, 293, 301, 309, 317, 325, 333, 341, 349, 357, 365, 373, 381, 389, 397, 405, 413, 421, 429, 437, 445, 453, 461, 469, 485, 493, 501, 525, 533, 541, 549, 557, 565, 613, 621, 685, 693, 701, 709, 717, 725, 733, 741, 749, 757, 765, 773, 781, 789, 797, 805, 813, 821, 829, 837, 845, 853, 861, 869, 877, 885, 893, 949, 957, 965, 981, 989, 997, 1005, 1013, 1021, 1029, 1037, 1045, 1053, 1069, 1077, 1085, 1093, 1101, 1109, 1117, 1125, 1133, 1141, 1149, 1157, 1165, 1173, 1181, 1189, 1197, 1205, 1213, 1221, 1229, 1237, 1245, 1253, 1261, 1269, 1277, 1285, 1293, 1301, 1309, 1317, 1325, 1333, 1341, 1349, 1357, 1365, 1373, 1381, 1389, 1397, 1405, 1413, 1421, 1429, 1437, 1445, 1453, 1461, 1469, 1477, 1485, 1493, 1501, 1509, 1517, 1525, 1533, 1549, 1557, 1565, 1573, 1581, 1589, 1597, 1605, 1613, 1621, 1629, 1637, 1653, 1661, 1677, 1685, 1693, 1701, 1709, 1717, 1725, 1733, 1741, 1749, 1757, 1765, 1773, 1781, 1789, 1797, 1805, 1813, 1821, 1837, 1845, 1853, 1861, 1869, 1877, 1885, 1893, 1917, 1941, 1949, 1957, 1965, 1973, 1981, 1989, 1997, 2005, 2013, 2021, 2029, 2037, 2045, 2053, 2061, 2069, 2077, 2085, 2093, 2101, 2109, 2117, 2125, 2133, 2141, 2149, 2157, 2165, 2173, 2181, 2189, 2197, 2205, 2213, 2221, 2229, 2237, 2245, 2253, 2261, 2269, 2277, 2285, 2301, 2309, 2317, 2325, 2333, 2341, and 2349; and/or
wherein the VL-1 or VL-3 comprise a V_{L} amino acid sequence selected from any one of SEQ ID NOs: 1, 9, 17, 25, 33, 41, 49, 57, 65, 73, 81, 89, 97, 105, 113, 121, 129, 145, 153, 161, 169, 177, 193, 201, 233, 241, 257, 273, 281, 289, 297, 305, 313, 321, 329, 337, 345, 353, 361, 369, 377, 385, 393, 401, 409, 417, 425, 433, 441, 449, 457, 465, 481, 489, 497, 521, 529, 537, 545, 553, 561, 609, 617, 681, 689, 697, 705, 713, 721, 729, 737, 745, 753, 761, 769, 777, 785, 793, 801, 809, 817, 825, 833, 841, 849, 857, 865, 873, 881, 889, 945, 953, 961, 977, 985, 993, 1001, 1009, 1017, 1025, 1033, 1041, 1049, 1065, 1073, 1081, 1089, 1097, 1105, 1113, 1121, 1129, 1137, 1145, 1153, 1161, 1169, 1177, 1185, 1193, 1201, 1209, 1217, 1225, 1233, 1241, 1249, 1257, 1265, 1273, 1281, 1289, 1297, 1305, 1313, 1321, 1329, 1337, 1345, 1353, 1361, 1369, 1377, 1385, 1393, 1401, 1409, 1417, 1425, 1433, 1441, 1449, 1457, 1465, 1473, 1481, 1489, 1497, 1505, 1513, 1521, 1529, 1545, 1553, 1561, 1569, 1577, 1585, 1593, 1601, 1609, 1617, 1625, 1633, 1649, 1657, 1673, 1681, 1689, 1697, 1705, 1713, 1721, 1729, 1737, 1745, 1753, 1761, 1769, 1777, 1785, 1793, 1801, 1809, 1817, 1833, 1841, 1849, 1857, 1865, 1873, 1881, 1889, 1913, 1937, 1945, 1953, 1961, 1969, 1977, 1985, 1993, 2001, 2009, 2017, 2025, 2033, 2041, 2049, 2057, 2065, 2073, 2081, 2089, 2097, 2105, 2113, 2121, 2129, 2137, 2145, 2153, 2161, 2169, 2177, 2185, 2193, 2201, 2209, 2217, 2225, 2233, 2241, 2249, 2257, 2265, 2273, 2281, 2297, 2305, 2313, 2321, 2329, 2337 and 2345, and/or
wherein VH-2 or VH-4 comprise a V_{H} amino acid sequence selected from any one of SEQ ID NOs: 21, 29, 37, 45, 125, 141, 173, 181, 189, 197, 205, 213, 221, 229, 237, 245, 253, 261, 269, 325, 333, 341, 397, 405, 413, 477, 485, 493, 501, 509, 517, 549, 557, 565, 573, 581, 589, 597, 605, 629, 637, 645, 653, 661, 669, 677, 685, 693, 701, 709, 717, 725, 733, 741, 749, 757, 765, 773, 789, 797, 805, 813, 821, 853, 861, 869, 877, 885, 893, 901, 909, 917, 925, 933, 941, 949, 973, 981, 1013, 1061, 1541, 1573, 1605, 1645, 1669, 1829, 1869, 1901, 1909, 1917, 1925, 1933, 2269, 2285, 2293, 2333, and 2349; and/or
wherein VL-2 or VL-4 comprise a V_{L} amino acid sequence selected from any one of SEQ ID NOs: 17, 25, 33, 41, 121, 137, 169, 177, 185, 193, 201, 209, 217, 225, 233, 241, 249, 257, 265, 321, 329, 337, 393, 401, 409, 473, 481, 489, 497, 505, 513, 545, 553, 561, 569, 577, 585, 593, 601, 625, 633, 641, 649, 657, 665, 673, 681, 689, 697, 705, 713, 721, 729, 737, 745, 753, 761, 769, 785, 793, 801, 809, 817, 849, 857, 865, 873, 881, 889, 897, 905, 913, 921, 929, 937, 945, 969, 977, 1009, 1057, 1537, 1569, 1601, 1641, 1665, 1825, 1865, 1897, 1905, 1913, 1921, 1929, 2265, 2281 2289, 2329, and 2345.

8. (canceled)

9. The heterodimeric multispecific antibody of claim 1, wherein each of VL-1 and VH-1 comprise a V_{L} amino acid sequence and a VH amino acid sequence selected from the group consisting of SEQ ID NOs: 1 and 5 respectively; SEQ ID NOs: 9 and 13 respectively; SEQ ID NOs: 17 and 21 respectively; SEQ ID NOs: 25 and 29 respectively; SEQ ID NOs: 33 and 37 respectively; SEQ ID NOs: 41 and 45 respectively; SEQ ID NOs: 49 and 53 respectively; SEQ ID NOs: 57 and 61 respectively; SEQ ID NOs: 73 and 77 respectively; SEQ ID NOs: 89 and 93 respectively; SEQ ID NOs: 97 and 101 respectively; SEQ ID NOs: 105 and 109 respectively; SEQ ID NOs: 113 and 117 respectively; SEQ ID NOs: 121 and 125 respectively; SEQ ID NOs: 129 and 133 respectively; SEQ ID NOs: 145 and 149 respectively; SEQ ID NOs: 161 and 165 respectively; SEQ ID NOs: 169 and 173 respectively; SEQ ID NOs: 177 and 181 respectively; SEQ ID NOs: 193 and 197 respectively; SEQ ID NOs: 201 and 205 respectively; SEQ ID NOs: 233 and 237 respectively; SEQ ID NOs: 241 and 245 respectively; SEQ ID NOs: 257 and 261 respectively; SEQ ID NOs: 273 and 277 respectively; SEQ ID NOs: 281 and 285 respectively; SEQ ID NOs: 289 and 293 respectively; SEQ ID NOs: 297 and 301 respectively; SEQ ID NOs: 305 and 309 respectively; SEQ ID NOs: 313 and 317 respectively; SEQ ID NOs: 321 and 325 respectively; SEQ ID NOs: 329 and 333 respectively; SEQ ID NOs: 337 and 341 respectively; SEQ ID NOs: 345 and 349 respectively; SEQ ID NOs: 353 and 357 respectively; SEQ ID NOs: 361 and 365 respectively; SEQ ID NOs: 369 and 373 respectively; SEQ ID NOs: 377 and 381 respectively; SEQ ID NOs: 385 and 389 respectively; SEQ ID NOs: 393 and 397 respectively; SEQ ID NOs: 401 and 405 respectively; SEQ ID NOs: 409 and 413 respectively; SEQ ID NOs: 417 and 421 respectively; SEQ ID NOs: 425 and 429 respectively; SEQ ID NOs: 433 and 437 respectively; SEQ ID NOs: 441 and 445 respectively; SEQ ID NOs: 449 and 453 respectively; SEQ ID NOs: 457 and 461 respectively; SEQ ID NOs: 465 and 469 respectively; SEQ ID NOs: 481 and 485 respectively; SEQ ID NOs: 489 and 493 respectively; SEQ ID NOs: 497 and 501 respectively; SEQ ID NOs: 521 and 525 respectively; SEQ ID NOs: 529 and 533 respectively; SEQ ID NOs: 537 and 541 respectively; SEQ ID NOs: 545 and 549 respectively; SEQ ID NOs: 553 and 557 respectively; SEQ ID NOs: 561 and 565 respectively; SEQ ID NOs: 609 and 613 respectively; SEQ ID NOs: 617 and 621 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 729 and 733 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 769 and 773 respectively; SEQ ID NOs: 785 and 789 respectively; SEQ ID NOs: 793 and 797 respectively; SEQ ID NOs: 801 and 805 respectively; SEQ ID NOs: 809 and 813 respectively; SEQ ID NOs: 817 and 821 respectively; SEQ ID NOs: 825 and 829 respectively; SEQ ID NOs: 833 and 837 respectively; SEQ ID NOs: 841 and 845 respectively; SEQ ID NOs: 849 and 853 respectively; SEQ ID NOs: 857 and 861 respectively; SEQ ID NOs: 865 and 869 respectively; SEQ ID NOs: 873 and 877 respectively; SEQ ID NOs: 881 and 885 respectively; SEQ ID NOs: 889 and 893 respectively; SEQ ID NOs: 945 and 949 respectively; SEQ ID NOs: 953 and 957 respectively; SEQ ID NOs: 961 and 965 respectively; SEQ ID NOs: 977 and 981 respectively; SEQ ID NOs: 985 and 989 respectively; SEQ ID NOs: 993 and 997 respectively; SEQ ID NOs: 1001 and 1005 respectively; SEQ ID NOs: 1009 and 1013 respectively; SEQ ID NOs: 1017 and 1021 respectively; SEQ ID NOs: 1025 and 1029 respectively; SEQ ID NOs: 1033 and 1037 respectively; SEQ ID NOs: 1041 and 1045 respectively; SEQ ID NOs: 1065 and 1069 respectively; SEQ ID NOs: 1073 and 1077 respectively; SEQ ID NOs: 1081 and 1085 respectively; SEQ ID NOs: 1089 and 1093 respectively; SEQ ID NOs: 1097 and 1101 respectively; SEQ ID NOs: 1113 and 1117 respectively; SEQ ID NOs: 1121 and 1125 respectively; SEQ ID NOs: 1129 and 1133 respectively; SEQ ID NOs: 1137 and 1141 respectively; SEQ ID NOs: 1145 and 1149 respectively; SEQ ID NOs: 1153 and 1157 respectively; SEQ ID NOs: 1161 and 1165 respectively; SEQ ID NOs: 1169 and 1173 respectively; SEQ ID NOs: 1185 and 1189 respectively; SEQ ID NOs: 1193 and 1197 respectively; SEQ ID NOs: 1201 and 1205 respectively; SEQ ID NOs: 1209 and 1213 respectively; SEQ ID NOs: 1217 and 1221 respectively; SEQ ID NOs: 1225 and 1229 respectively; SEQ ID NOs: 1233 and 1237 respectively; SEQ ID NOs: 1241 and 1245 respectively; SEQ ID NOs: 1249 and 1253 respectively; SEQ ID NOs: 1257 and 1261 respectively; SEQ ID NOs: 1265 and 1269 respectively; SEQ ID NOs: 1273 and 1277 respectively; SEQ ID NOs: 1281 and 1285 respectively; SEQ ID NOs: 1289 and 1293 respectively; SEQ ID NOs: 1297 and 1301 respectively; SEQ ID NOs: 1305 and 1309 respectively; SEQ ID NOs: 1313 and 1317 respectively; SEQ ID NOs: 1321 and 1325 respectively; SEQ ID NOs: 1329 and 1333 respectively; SEQ ID NOs: 1337 and 1341 respectively; SEQ ID NOs: 1345 and 1349 respectively; SEQ ID NOs: 1353 and 1357 respectively; SEQ ID NOs: 1361 and 1365 respectively; SEQ ID NOs: 1369 and 1373 respectively; SEQ ID NOs: 1377 and 1381 respectively; SEQ ID NOs: 1385 and 1389 respectively; SEQ ID NOs: 1393 and 1397 respectively; SEQ ID NOs: 1401 and 1405 respectively; SEQ ID NOs: 1409 and 1413 respectively; SEQ ID NOs: 1417 and 1421 respectively; SEQ ID NOs: 1433 and 1437 respectively; SEQ ID NOs: 1441 and 1445 respectively; SEQ ID NOs: 1457 and 1461 respectively; SEQ ID NOs: 1465 and 1469 respectively; SEQ ID NOs: 1473 and 1477 respectively; SEQ ID NOs: 1481 and 1485 respectively; SEQ ID NOs: 1489 and 1493 respectively; SEQ ID NOs: 1497 and 1501 respectively; SEQ ID NOs: 1505 and 1509 respectively; SEQ ID NOs: 1513 and 1517 respectively; SEQ ID NOs: 1521 and 1525 respectively; SEQ ID NOs: 1529 and 1533 respectively; SEQ ID NOs: 1545 and 1549 respectively; SEQ ID NOs: 1553 and 1557 respectively; SEQ ID NOs: 1561 and 1565 respectively; SEQ ID NOs: 1569 and 1573 respectively; SEQ ID NOs: 1577 and 1581 respectively; SEQ ID NOs: 1585 and 1589 respectively; SEQ ID NOs: 1593 and 1597 respectively; SEQ ID NOs: 1601 and 1605 respectively; SEQ ID NOs: 1609 and 1613 respectively; SEQ ID NOs: 1617 and 1621 respectively; SEQ ID NOs: 1625 and 1629 respectively; SEQ ID NOs: 1633 and 1637 respectively; SEQ ID NOs: 1649 and 1653 respectively; SEQ ID NOs: 1657 and 1661 respectively; SEQ ID NOs: 1673 and 1677 respectively; SEQ ID NOs: 1681 and 1685 respectively; SEQ ID NOs: 1689 and 1693 respectively; SEQ ID NOs: 1697 and 1701 respectively; SEQ ID NOs: 1705 and 1709 respectively; SEQ ID NOs: 1713 and 1717 respectively; SEQ ID NOs: 1721 and 1725 respectively; SEQ ID NOs: 1729 and 1733 respectively; SEQ ID NOs: 1737 and 1741 respectively; SEQ ID NOs: 1745 and 1749 respectively; SEQ ID NOs: 1753 and 1757 respectively; SEQ ID NOs: 1761 and 1765 respectively; SEQ ID NOs: 1769 and 1773 respectively; SEQ ID NOs: 1777 and 1781 respectively; SEQ ID NOs: 1785 and 1789 respectively; SEQ ID NOs: 1793 and 1797 respectively; SEQ ID NOs: 1801 and 1805 respectively; SEQ ID NOs: 1809 and 1813 respectively; SEQ ID NOs: 1817 and 1821 respectively; SEQ ID NOs: 1833 and 1837 respectively; SEQ ID NOs: 1841 and 1845 respectively; SEQ ID NOs: 1849 and 1853 respectively; SEQ ID NOs: 1857 and 1861 respectively; SEQ ID NOs: 1865 and 1869 respectively; SEQ ID NOs: 1873 and 1877 respectively; SEQ ID NOs: 1881 and 1885 respectively; SEQ ID NOs: 1889 and 1893 respectively; SEQ ID NOs: 1913 and 1917 respectively; SEQ ID NOs: 1937 and 1941 respectively; SEQ ID NOs: 1945 and 1949 respectively; SEQ ID NOs: 1953 and 1957 respectively; SEQ ID NOs: 1961 and 1965 respectively; SEQ ID NOs: 1969 and 1973 respectively; SEQ ID NOs: 1977 and 1981 respectively; SEQ ID NOs: 1985 and 1989 respectively; SEQ ID NOs: 1993 and 1997 respectively; SEQ ID NOs: 2001 and 2005 respectively; SEQ ID NOs: 2009 and 2013 respectively; SEQ ID NOs: 2017 and 2021 respectively; SEQ ID NOs: 2025 and 2029 respectively; SEQ ID NOs: 2033 and 2037 respectively; SEQ ID NOs: 2041 and 2045 respectively; SEQ ID NOs: 2049 and 2053 respectively; SEQ ID NOs: 2057 and 2061 respectively; SEQ ID NOs: 2065 and 2069 respectively; SEQ ID NOs: 2073 and 2077 respectively; SEQ ID NOs: 2081 and 2085 respectively; SEQ ID NOs: 2089 and 2093 respectively; SEQ ID NOs: 2097 and 2101 respectively; SEQ ID NOs: 2105 and 2109 respectively; SEQ ID NOs: 2113 and 2117 respectively; SEQ ID NOs: 2121 and 2125 respectively; SEQ ID NOs: 2129 and 2133 respectively; SEQ ID NOs: 2137 and 2141 respectively; SEQ ID NOs: 2145 and 2149 respectively; SEQ ID NOs: 2153 and 2157 respectively; SEQ ID NOs: 2161 and 2165 respectively; SEQ ID NOs: 2169 and 2173 respectively; SEQ ID NOs: 2177 and 2181 respectively; SEQ ID NOs: 2185 and 2189 respectively; SEQ ID NOs: 2193 and 2197 respectively; SEQ ID NOs: 2201 and 2205 respectively; SEQ ID NOs: 2209 and 2213 respectively; SEQ ID NOs: 2217 and 2221 respectively; SEQ ID NOs: 2225 and 2229 respectively; SEQ ID NOs: 2233 and 2237 respectively; SEQ ID NOs: 2241 and 2245 respectively; SEQ ID NOs: 2249 and 2253 respectively; SEQ ID NOs: 2257 and 2261 respectively; SEQ ID NOs: 2273 and 2277 respectively; SEQ ID NOs: 2281 and 2285 respectively; SEQ ID NOs: 2305 and 2309 respectively; SEQ ID NOs: 2313 and 2317 respectively; SEQ ID NOs: 2321 and 2325 respectively; SEQ ID NOs: 2329 and 2333 respectively; SEQ ID NOs: 2337 and 2341 respectively; and SEQ ID NOs: 2345 and 2349 respectively, or
wherein each of VL-3 and VH-3 comprise a V_{L} amino acid sequence and a V_{H} amino acid sequence selected from the group consisting of SEQ ID NOs: 1 and 5 respectively; SEQ ID NOs: 9 and 13 respectively; SEQ ID NOs: 17 and 21 respectively; SEQ ID NOs: 25 and 29 respectively; SEQ ID NOs: 33 and 37 respectively; SEQ ID NOs: 41 and 45 respectively; SEQ ID NOs: 49 and 53 respectively; SEQ ID NOs: 57 and 61 respectively; SEQ ID NOs: 73 and 77 respectively; SEQ ID NOs: 89 and 93 respectively; SEQ ID NOs: 97 and 101 respectively; SEQ ID NOs: 105 and 109 respectively; SEQ ID NOs: 113 and 117 respectively; SEQ ID NOs: 121 and 125 respectively; SEQ ID NOs: 129 and 133 respectively; SEQ ID NOs: 145 and 149 respectively; SEQ ID NOs: 161 and 165 respectively; SEQ ID NOs: 169 and 173 respectively; SEQ ID NOs: 177 and 181 respectively; SEQ ID NOs: 193 and 197 respectively; SEQ ID NOs: 201 and 205 respectively; SEQ ID NOs: 233 and 237 respectively; SEQ ID NOs: 241 and 245 respectively; SEQ ID NOs: 257 and 261 respectively; SEQ ID NOs: 273 and 277 respectively; SEQ ID NOs: 281 and 285 respectively; SEQ ID NOs: 289 and 293 respectively; SEQ ID NOs: 297 and 301 respectively; SEQ ID NOs: 305 and 309 respectively; SEQ ID NOs: 313 and 317 respectively; SEQ ID NOs: 321 and 325 respectively; SEQ ID NOs: 329 and 333 respectively; SEQ ID NOs: 337 and 341 respectively; SEQ ID NOs: 345 and 349 respectively; SEQ ID NOs: 353 and 357 respectively; SEQ ID NOs: 361 and 365 respectively; SEQ ID NOs: 369 and 373 respectively; SEQ ID NOs: 377 and 381 respectively; SEQ ID NOs: 385 and 389 respectively; SEQ ID NOs: 393 and 397 respectively; SEQ ID NOs: 401 and 405 respectively; SEQ ID NOs: 409 and 413 respectively; SEQ ID NOs: 417 and 421 respectively; SEQ ID NOs: 425 and 429 respectively; SEQ ID NOs: 433 and 437 respectively; SEQ ID NOs: 441 and 445 respectively; SEQ ID NOs: 449 and 453 respectively; SEQ ID NOs: 457 and 461 respectively; SEQ ID NOs: 465 and 469 respectively; SEQ ID NOs: 481 and 485 respectively; SEQ ID NOs: 489 and 493 respectively; SEQ ID NOs: 497 and 501 respectively; SEQ ID NOs: 521 and 525 respectively; SEQ ID NOs: 529 and 533 respectively; SEQ ID NOs: 537 and 541 respectively; SEQ ID NOs: 545 and 549 respectively; SEQ ID NOs: 553 and 557 respectively; SEQ ID NOs: 561 and 565 respectively; SEQ ID NOs: 609 and 613 respectively; SEQ ID NOs: 617 and 621 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 729 and 733 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 769 and 773 respectively; SEQ ID NOs: 785 and 789 respectively; SEQ ID NOs: 793 and 797 respectively; SEQ ID NOs: 801 and 805 respectively; SEQ ID NOs: 809 and 813 respectively; SEQ ID NOs: 817 and 821 respectively; SEQ ID NOs: 825 and 829 respectively; SEQ ID NOs: 833 and 837 respectively; SEQ ID NOs: 841 and 845 respectively; SEQ ID NOs: 849 and 853 respectively; SEQ ID NOs: 857 and 861 respectively; SEQ ID NOs: 865 and 869 respectively; SEQ ID NOs: 873 and 877 respectively; SEQ ID NOs: 881 and 885 respectively; SEQ ID NOs: 889 and 893 respectively; SEQ ID NOs: 945 and 949 respectively; SEQ ID NOs: 953 and 957 respectively; SEQ ID NOs: 961 and 965 respectively; SEQ ID NOs: 977 and 981 respectively; SEQ ID NOs: 985 and 989 respectively; SEQ ID NOs: 993 and 997 respectively; SEQ ID NOs: 1001 and 1005 respectively; SEQ ID NOs: 1009 and 1013 respectively; SEQ ID NOs: 1017 and 1021 respectively; SEQ ID NOs: 1025 and 1029 respectively; SEQ ID NOs: 1033 and 1037 respectively; SEQ ID NOs: 1041 and 1045 respectively; SEQ ID NOs: 1065 and 1069 respectively; SEQ ID NOs: 1073 and 1077 respectively; SEQ ID NOs: 1081 and 1085 respectively; SEQ ID NOs: 1089 and 1093 respectively; SEQ ID NOs: 1097 and 1101 respectively; SEQ ID NOs: 1113 and 1117 respectively; SEQ ID NOs: 1121 and 1125 respectively; SEQ ID NOs: 1129 and 1133 respectively; SEQ ID NOs: 1137 and 1141 respectively; SEQ ID NOs: 1145 and 1149 respectively; SEQ ID NOs: 1153 and 1157 respectively; SEQ ID NOs: 1161 and 1165 respectively; SEQ ID NOs: 1169 and 1173 respectively; SEQ ID NOs: 1185 and 1189 respectively; SEQ ID NOs: 1193 and 1197 respectively; SEQ ID NOs: 1201 and 1205 respectively; SEQ ID NOs: 1209 and 1213 respectively; SEQ ID NOs: 1217 and 1221 respectively; SEQ ID NOs: 1225 and 1229 respectively; SEQ ID NOs: 1233 and 1237 respectively; SEQ ID NOs: 1241 and 1245 respectively; SEQ ID NOs: 1249 and 1253 respectively; SEQ ID NOs: 1257 and 1261 respectively; SEQ ID NOs: 1265 and 1269 respectively; SEQ ID NOs: 1273 and 1277 respectively; SEQ ID NOs: 1281 and 1285 respectively; SEQ ID NOs: 1289 and 1293 respectively; SEQ ID NOs: 1297 and 1301 respectively; SEQ ID NOs: 1305 and 1309 respectively; SEQ ID NOs: 1313 and 1317 respectively; SEQ ID NOs: 1321 and 1325 respectively; SEQ ID NOs: 1329 and 1333 respectively; SEQ ID NOs: 1337 and 1341 respectively; SEQ ID NOs: 1345 and 1349 respectively; SEQ ID NOs: 1353 and 1357 respectively; SEQ ID NOs: 1361 and 1365 respectively; SEQ ID NOs: 1369 and 1373 respectively; SEQ ID NOs: 1377 and 1381 respectively; SEQ ID NOs: 1385 and 1389 respectively; SEQ ID NOs: 1393 and 1397 respectively; SEQ ID NOs: 1401 and 1405 respectively; SEQ ID NOs: 1409 and 1413 respectively; SEQ ID NOs: 1417 and 1421 respectively; SEQ ID NOs: 1433 and 1437 respectively; SEQ ID NOs: 1441 and 1445 respectively; SEQ ID NOs: 1457 and 1461 respectively; SEQ ID NOs: 1465 and 1469 respectively; SEQ ID NOs: 1473 and 1477 respectively; SEQ ID NOs: 1481 and 1485 respectively; SEQ ID NOs: 1489 and 1493 respectively; SEQ ID NOs: 1497 and 1501 respectively; SEQ ID NOs: 1505 and 1509 respectively; SEQ ID NOs: 1513 and 1517 respectively; SEQ ID NOs: 1521 and 1525 respectively; SEQ ID NOs: 1529 and 1533 respectively; SEQ ID NOs: 1545 and 1549 respectively; SEQ ID NOs: 1553 and 1557 respectively; SEQ ID NOs: 1561 and 1565 respectively; SEQ ID NOs: 1569 and 1573 respectively; SEQ ID NOs: 1577 and 1581 respectively; SEQ ID NOs: 1585 and 1589 respectively; SEQ ID NOs: 1593 and 1597 respectively; SEQ ID NOs: 1601 and 1605 respectively; SEQ ID NOs: 1609 and 1613 respectively; SEQ ID NOs: 1617 and 1621 respectively; SEQ ID NOs: 1625 and 1629 respectively; SEQ ID NOs: 1633 and 1637 respectively; SEQ ID NOs: 1649 and 1653 respectively; SEQ ID NOs: 1657 and 1661 respectively; SEQ ID NOs: 1673 and 1677 respectively; SEQ ID NOs: 1681 and 1685 respectively; SEQ ID NOs: 1689 and 1693 respectively; SEQ ID NOs: 1697 and 1701 respectively; SEQ ID NOs: 1705 and 1709 respectively; SEQ ID NOs: 1713 and 1717 respectively; SEQ ID NOs: 1721 and 1725 respectively; SEQ ID NOs: 1729 and 1733 respectively; SEQ ID NOs: 1737 and 1741 respectively; SEQ ID NOs: 1745 and 1749 respectively; SEQ ID NOs: 1753 and 1757 respectively; SEQ ID NOs: 1761 and 1765 respectively; SEQ ID NOs: 1769 and 1773 respectively; SEQ ID NOs: 1777 and 1781 respectively; SEQ ID NOs: 1785 and 1789 respectively; SEQ ID NOs: 1793 and 1797 respectively; SEQ ID NOs: 1801 and 1805 respectively; SEQ ID NOs: 1809 and 1813 respectively; SEQ ID NOs: 1817 and 1821 respectively; SEQ ID NOs: 1833 and 1837 respectively; SEQ ID NOs: 1841 and 1845 respectively; SEQ ID NOs: 1849 and 1853 respectively; SEQ ID NOs: 1857 and 1861 respectively; SEQ ID NOs: 1865 and 1869 respectively; SEQ ID NOs: 1873 and 1877 respectively; SEQ ID NOs: 1881 and 1885 respectively; SEQ ID NOs: 1889 and 1893 respectively; SEQ ID NOs: 1913 and 1917 respectively; SEQ ID NOs: 1937 and 1941 respectively; SEQ ID NOs: 1945 and 1949 respectively; SEQ ID NOs: 1953 and 1957 respectively; SEQ ID NOs: 1961 and 1965 respectively; SEQ ID NOs: 1969 and 1973 respectively; SEQ ID NOs: 1977 and 1981 respectively; SEQ ID NOs: 1985 and 1989 respectively; SEQ ID NOs: 1993 and 1997 respectively; SEQ ID NOs: 2001 and 2005 respectively; SEQ ID NOs: 2009 and 2013 respectively; SEQ ID NOs: 2017 and 2021 respectively; SEQ ID NOs: 2025 and 2029 respectively; SEQ ID NOs: 2033 and 2037 respectively; SEQ ID NOs: 2041 and 2045 respectively; SEQ ID NOs: 2049 and 2053 respectively; SEQ ID NOs: 2057 and 2061 respectively; SEQ ID NOs: 2065 and 2069 respectively; SEQ ID NOs: 2073 and 2077 respectively; SEQ ID NOs: 2081 and 2085 respectively; SEQ ID NOs: 2089 and 2093 respectively; SEQ ID NOs: 2097 and 2101 respectively; SEQ ID NOs: 2105 and 2109 respectively; SEQ ID NOs: 2113 and 2117 respectively; SEQ ID NOs: 2121 and 2125 respectively; SEQ ID NOs: 2129 and 2133 respectively; SEQ ID NOs: 2137 and 2141 respectively; SEQ ID NOs: 2145 and 2149 respectively; SEQ ID NOs: 2153 and 2157 respectively; SEQ ID NOs: 2161 and 2165 respectively; SEQ ID NOs: 2169 and 2173 respectively; SEQ ID NOs: 2177 and 2181 respectively; SEQ ID NOs: 2185 and 2189 respectively; SEQ ID NOs: 2193 and 2197 respectively; SEQ ID NOs: 2201 and 2205 respectively; SEQ ID NOs: 2209 and 2213 respectively; SEQ ID NOs: 2217 and 2221 respectively; SEQ ID NOs: 2225 and 2229 respectively; SEQ ID NOs: 2233 and 2237 respectively; SEQ ID NOs: 2241 and 2245 respectively; SEQ ID NOs: 2249 and 2253 respectively; SEQ ID NOs: 2257 and 2261 respectively; SEQ ID NOs: 2273 and 2277 respectively; SEQ ID NOs: 2281 and 2285 respectively; SEQ ID NOs: 2305 and 2309 respectively; SEQ ID NOs: 2313 and 2317 respectively; SEQ ID NOs: 2321 and 2325 respectively; SEQ ID NOs: 2329 and 2333 respectively; SEQ ID NOs: 2337 and 2341 respectively; and SEQ ID NOs: 2345 and 2349 respectively.

10. (canceled)

11. The heterodimeric multispecific antibody of claim 1, wherein each of VL-1 and VH-1 comprise a V_{L} amino acid sequence and a V_{H} amino acid sequence selected from the group consisting of SEQ ID NOs: 9 and 13 respectively; SEQ ID NOs: 49 and 53 respectively; SEQ ID NOs: 57 and 61 respectively; SEQ ID NOs: 65 and 69 respectively; SEQ ID NOs: 81 and 85 respectively; SEQ ID NOs: 153 and 157 respectively; SEQ ID NOs: 161 and 165 respectively; SEQ ID NOs: 193 and 197 respectively; SEQ ID NOs: 201 and 205 respectively; SEQ ID NOs: 273 and 277 respectively; SEQ ID NOs: 281 and 285 respectively; SEQ ID NOs: 289 and 293 respectively; SEQ ID NOs: 297 and 301 respectively; SEQ ID NOs: 305 and 309 respectively; SEQ ID NOs: 313 and 317 respectively; SEQ ID NOs: 361 and 365 respectively; SEQ ID NOs: 377 and 381 respectively; SEQ ID NOs: 393 and 397 respectively; SEQ ID NOs: 401 and 405 respectively; SEQ ID NOs: 409 and 413 respectively; SEQ ID NOs: 417 and 421 respectively; SEQ ID NOs: 425 and 429 respectively; SEQ ID NOs: 433 and 437 respectively; SEQ ID NOs: 441 and 445 respectively; SEQ ID NOs: 449 and 453 respectively; SEQ ID NOs: 457 and 461 respectively; SEQ ID NOs: 465 and 469 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 777 and 781 respectively; SEQ ID NOs: 825 and 829 respectively; SEQ ID NOs: 833 and 837 respectively; SEQ ID NOs: 841 and 845 respectively; SEQ ID NOs: 953 and 957 respectively; SEQ ID NOs: 961 and 965 respectively; SEQ ID NOs: 977 and 981 respectively; SEQ ID NOs: 993 and 997 respectively; SEQ ID NOs: 1001 and 1005 respectively; SEQ ID NOs: 1009 and 1013 respectively; SEQ ID NOs: 1017 and 1021 respectively; SEQ ID NOs: 1033 and 1037 respectively; SEQ ID NOs: 1049 and 1053 respectively; SEQ ID NOs: 1073 and 1077 respectively; SEQ ID NOs: 1081 and 1085 respectively; SEQ ID NOs: 1089 and 1093 respectively; SEQ ID NOs: 1105 and 1109 respectively; SEQ ID NOs: 1129 and 1133 respectively; SEQ ID NOs: 1137 and 1141 respectively; SEQ ID NOs: 1153 and 1157 respectively; SEQ ID NOs: 1161 and 1165 respectively; SEQ ID NOs: 1177 and 1181 respectively; SEQ ID NOs: 1225 and 1229 respectively; SEQ ID NOs: 1241 and 1245 respectively; SEQ ID NOs: 1257 and 1261 respectively; SEQ ID NOs: 1265 and 1269 respectively; SEQ ID NOs: 1297 and 1301 respectively; SEQ ID NOs: 1393 and 1397 respectively; SEQ ID NOs: 1409 and 1413 respectively; SEQ ID NOs: 1425 and 1429 respectively; SEQ ID NOs: 1441 and 1445 respectively; SEQ ID NOs: 1449 and 1453 respectively; SEQ ID NOs: 1457 and 1461 respectively; SEQ ID NOs: 1465 and 1469 respectively; SEQ ID NOs: 1473 and 1477 respectively; SEQ ID NOs: 1481 and 1485 respectively; SEQ ID NOs: 1497 and 1501 respectively; SEQ ID NOs: 1505 and 1509 respectively; SEQ ID NOs: 1513 and 1517 respectively; SEQ ID NOs: 1521 and 1525 respectively; SEQ ID NOs: 1529 and 1533 respectively; SEQ ID NOs: 1545 and 1549 respectively; SEQ ID NOs: 1553 and 1557 respectively; SEQ ID NOs: 1561 and 1565 respectively; SEQ ID NOs: 1569 and 1573 respectively; SEQ ID NOs: 1577 and 1581 respectively; SEQ ID NOs: 1585 and 1589 respectively; SEQ ID NOs: 1609 and 1613 respectively; SEQ ID NOs: 1617 and 1621 respectively; SEQ ID NOs: 1649 and 1653 respectively; SEQ ID NOs: 1657 and 1661 respectively; SEQ ID NOs: 1673 and 1677 respectively; SEQ ID NOs: 1689 and 1693 respectively; SEQ ID NOs: 1697 and 1701 respectively; SEQ ID NOs: 1705 and 1709 respectively; SEQ ID NOs: 1713 and 1717 respectively; SEQ ID NOs: 1721 and 1725 respectively; SEQ ID NOs: 1729 and 1733 respectively; SEQ ID NOs: 1745 and 1749 respectively; SEQ ID NOs: 1753 and 1757 respectively; SEQ ID NOs: 1761 and 1765 respectively; SEQ ID NOs: 1769 and 1773 respectively; SEQ ID NOs: 1777 and 1781 respectively; SEQ ID NOs: 1785 and 1789 respectively; SEQ ID NOs: 1793 and 1797 respectively; SEQ ID NOs: 1817 and 1821 respectively; SEQ ID NOs: 1833 and 1837 respectively; SEQ ID NOs: 1841 and 1845 respectively; SEQ ID NOs: 1849 and 1853 respectively; SEQ ID NOs: 1857 and 1861 respectively; SEQ ID NOs: 1865 and 1869 respectively; SEQ ID NOs: 1889 and 1893 respectively; SEQ ID NOs: 2257 and 2261 respectively; SEQ ID NOs: 2265 and 2269 respectively; SEQ ID NOs: 2281 and 2285 respectively; SEQ ID NOs: 2297 and 2301 respectively; SEQ ID NOs: 2305 and 2309 respectively; SEQ ID NOs: 2313 and 2317 respectively; SEQ ID NOs: 2321 and 2325 respectively; SEQ ID NOs: 2329 and 2333 respectively; SEQ ID NOs: 2337 and 2341 respectively; and SEQ ID NOs: 2345 and 2349 respectively.

12. The heterodimeric multispecific antibody of claim 1, wherein each of VL-3 and VH-3 comprise a V_{L} amino acid sequence and a V_{H} amino acid sequence selected from the group consisting of SEQ ID NOs: 9 and 13 respectively; SEQ ID NOs: 49 and 53 respectively; SEQ ID NOs: 57 and 61 respectively; SEQ ID NOs: 65 and 69 respectively; SEQ ID NOs: 81 and 85 respectively; SEQ ID NOs: 153 and 157 respectively; SEQ ID NOs: 161 and 165 respectively; SEQ ID NOs: 193 and 197 respectively; SEQ ID NOs: 201 and 205 respectively; SEQ ID NOs: 273 and 277 respectively; SEQ ID NOs: 281 and 285 respectively; SEQ ID NOs: 289 and 293 respectively; SEQ ID NOs: 297 and 301 respectively; SEQ ID NOs: 305 and 309 respectively; SEQ ID NOs: 313 and 317 respectively; SEQ ID NOs: 361 and 365 respectively; SEQ ID NOs: 377 and 381 respectively; SEQ ID NOs: 393 and 397 respectively; SEQ ID NOs: 401 and 405 respectively; SEQ ID NOs: 409 and 413 respectively; SEQ ID NOs: 417 and 421 respectively; SEQ ID NOs: 425 and 429 respectively; SEQ ID NOs: 433 and 437 respectively; SEQ ID NOs: 441 and 445 respectively; SEQ ID NOs: 449 and 453 respectively; SEQ ID NOs: 457 and 461 respectively; SEQ ID NOs: 465 and 469 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 777 and 781 respectively; SEQ ID NOs: 825 and 829 respectively; SEQ ID NOs: 833 and 837 respectively; SEQ ID NOs: 841 and 845 respectively; SEQ ID NOs: 953 and 957 respectively; SEQ ID NOs: 961 and 965 respectively; SEQ ID NOs: 977 and 981 respectively; SEQ ID NOs: 993 and 997 respectively; SEQ ID NOs: 1001 and 1005 respectively; SEQ ID NOs: 1009 and 1013 respectively; SEQ ID NOs: 1017 and 1021 respectively; SEQ ID NOs: 1033 and 1037 respectively; SEQ ID NOs: 1049 and 1053 respectively; SEQ ID NOs: 1073 and 1077 respectively; SEQ ID NOs: 1081 and 1085 respectively; SEQ ID NOs: 1089 and 1093 respectively; SEQ ID NOs: 1105 and 1109 respectively; SEQ ID NOs: 1129 and 1133 respectively; SEQ ID NOs: 1137 and 1141 respectively; SEQ ID NOs: 1153 and 1157 respectively; SEQ ID NOs: 1161 and 1165 respectively; SEQ ID NOs: 1177 and 1181 respectively; SEQ ID NOs: 1225 and 1229 respectively; SEQ ID NOs: 1241 and 1245 respectively; SEQ ID NOs: 1257 and 1261 respectively; SEQ ID NOs: 1265 and 1269 respectively; SEQ ID NOs: 1297 and 1301 respectively; SEQ ID NOs: 1393 and 1397 respectively; SEQ ID NOs: 1409 and 1413 respectively; SEQ ID NOs: 1425 and 1429 respectively; SEQ ID NOs: 1441 and 1445 respectively; SEQ ID NOs: 1449 and 1453 respectively; SEQ ID NOs: 1457 and 1461 respectively; SEQ ID NOs: 1465 and 1469 respectively; SEQ ID NOs: 1473 and 1477 respectively; SEQ ID NOs: 1481 and 1485 respectively; SEQ ID NOs: 1497 and 1501 respectively; SEQ ID NOs: 1505 and 1509 respectively; SEQ ID NOs: 1513 and 1517 respectively; SEQ ID NOs: 1521 and 1525 respectively; SEQ ID NOs: 1529 and 1533 respectively; SEQ ID NOs: 1545 and 1549 respectively; SEQ ID NOs: 1553 and 1557 respectively; SEQ ID NOs: 1561 and 1565 respectively; SEQ ID NOs: 1569 and 1573 respectively; SEQ ID NOs: 1577 and 1581 respectively; SEQ ID NOs: 1585 and 1589 respectively; SEQ ID NOs: 1609 and 1613 respectively; SEQ ID NOs: 1617 and 1621 respectively; SEQ ID NOs: 1649 and 1653 respectively; SEQ ID NOs: 1657 and 1661 respectively; SEQ ID NOs: 1673 and 1677 respectively; SEQ ID NOs: 1689 and 1693 respectively; SEQ ID NOs: 1697 and 1701 respectively; SEQ ID NOs: 1705 and 1709 respectively; SEQ ID NOs: 1713 and 1717 respectively; SEQ ID NOs: 1721 and 1725 respectively; SEQ ID NOs: 1729 and 1733 respectively; SEQ ID NOs: 1745 and 1749 respectively; SEQ ID NOs: 1753 and 1757 respectively; SEQ ID NOs: 1761 and 1765 respectively; SEQ ID NOs: 1769 and 1773 respectively; SEQ ID NOs: 1777 and 1781 respectively; SEQ ID NOs: 1785 and 1789 respectively; SEQ ID NOs: 1793 and 1797 respectively; SEQ ID NOs: 1817 and 1821 respectively; SEQ ID NOs: 1833 and 1837 respectively; SEQ ID NOs: 1841 and 1845 respectively; SEQ ID NOs: 1849 and 1853 respectively; SEQ ID NOs: 1857 and 1861 respectively; SEQ ID NOs: 1865 and 1869 respectively; SEQ ID NOs: 1889 and 1893 respectively; SEQ ID NOs: 2257 and 2261 respectively; SEQ ID NOs: 2265 and 2269 respectively; SEQ ID NOs: 2281 and 2285 respectively; SEQ ID NOs: 2297 and 2301 respectively; SEQ ID NOs: 2305 and 2309 respectively; SEQ ID NOs: 2313 and 2317 respectively; SEQ ID NOs: 2321 and 2325 respectively; SEQ ID NOs: 2329 and 2333 respectively; SEQ ID NOs: 2337 and 2341 respectively; and SEQ ID NOs: 2345 and 2349 respectively.

13. (canceled)

14. The heterodimeric multispecific antibody of claim 1, wherein each of VL-4 and VH-4 comprise a V_{L} amino acid sequence and a V_{H} amino acid sequence selected from the group consisting of SEQ ID NOs: 17 and 21 respectively; SEQ ID NOs: 25 and 29 respectively; SEQ ID NOs: 33 and 37 respectively; SEQ ID NOs: 41 and 45 respectively; SEQ ID NOs: 121 and 125 respectively; SEQ ID NOs: 137 and 141 respectively; SEQ ID NOs: 169 and 173 respectively; SEQ ID NOs: 177 and 181 respectively; SEQ ID NOs: 185 and 189 respectively; SEQ ID NOs: 193 and 197 respectively; SEQ ID NOs: 201 and 205 respectively; SEQ ID NOs: 209 and 213 respectively; SEQ ID NOs: 217 and 221 respectively; SEQ ID NOs: 225 and 229 respectively; SEQ ID NOs: 233 and 237 respectively; SEQ ID NOs: 241 and 245 respectively; SEQ ID NOs: 249 and 253 respectively; SEQ ID NOs: 257 and 261 respectively; SEQ ID NOs: 265 and 269 respectively; SEQ ID NOs: 321 and 325 respectively; SEQ ID NOs: 329 and 333 respectively; SEQ ID NOs: 337 and 341 respectively; SEQ ID NOs: 393 and 397 respectively; SEQ ID NOs: 401 and 405 respectively; SEQ ID NOs: 409 and 413 respectively; SEQ ID NOs: 473 and 477 respectively; SEQ ID NOs: 481 and 485 respectively; SEQ ID NOs: 489 and 493 respectively; SEQ ID NOs: 497 and 501 respectively; SEQ ID NOs: 505 and 509 respectively; SEQ ID NOs: 513 and 517 respectively; SEQ ID NOs: 545 and 549 respectively; SEQ ID NOs: 553 and 557 respectively; SEQ ID NOs: 561 and 565 respectively; SEQ ID NOs: 569 and 573 respectively; SEQ ID NOs: 577 and 581 respectively; SEQ ID NOs: 585 and 589 respectively; SEQ ID NOs: 593 and 597 respectively; SEQ ID NOs: 601 and 605 respectively; SEQ ID NOs: 625 and 629 respectively; SEQ ID NOs: 633 and 637 respectively; SEQ ID NOs: 641 and 645 respectively; SEQ ID NOs: 649 and 653 respectively; SEQ ID NOs: 657 and 661 respectively; SEQ ID NOs: 665 and 669 respectively; SEQ ID NOs: 673 and 677 respectively; SEQ ID NOs: 681 and 685 respectively; SEQ ID NOs: 689 and 693 respectively; SEQ ID NOs: 697 and 701 respectively; SEQ ID NOs: 705 and 709 respectively; SEQ ID NOs: 713 and 717 respectively; SEQ ID NOs: 721 and 725 respectively; SEQ ID NOs: 729 and 733 respectively; SEQ ID NOs: 737 and 741 respectively; SEQ ID NOs: 745 and 749 respectively; SEQ ID NOs: 753 and 757 respectively; SEQ ID NOs: 761 and 765 respectively; SEQ ID NOs: 769 and 773 respectively; SEQ ID NOs: 785 and 789 respectively; SEQ ID NOs: 793 and 797 respectively; SEQ ID NOs: 801 and 805 respectively; SEQ ID NOs: 809 and 813 respectively; SEQ ID NOs: 817 and 821 respectively; SEQ ID NOs: 849 and 853 respectively; SEQ ID NOs: 857 and 861 respectively; SEQ ID NOs: 865 and 869 respectively; SEQ ID NOs: 873 and 877 respectively; SEQ ID NOs: 881 and 885 respectively; SEQ ID NOs: 889 and 893 respectively; SEQ ID NOs: 897 and 901 respectively; SEQ ID NOs: 905 and 909 respectively; SEQ ID NOs: 913 and 917 respectively; SEQ ID NOs: 921 and 925 respectively; SEQ ID NOs: 929 and 933 respectively; SEQ ID NOs: 937 and 941 respectively; SEQ ID NOs: 945 and 949 respectively; SEQ ID NOs: 969 and 973 respectively; SEQ ID NOs: 977 and 981 respectively; SEQ ID NOs: 1009 and 1013 respectively; SEQ ID NOs: 1057 and 1061 respectively; SEQ ID NOs: 1537 and 1541 respectively; SEQ ID NOs: 1569 and 1573 respectively; SEQ ID NOs: 1601 and 1605 respectively; SEQ ID NOs: 1641 and 1645 respectively; SEQ ID NOs: 1665 and 1669 respectively; SEQ ID NOs: 1825 and 1829 respectively; SEQ ID NOs: 1865 and 1869 respectively; SEQ ID NOs: 1897 and 1901 respectively; SEQ ID NOs: 1905 and 1909 respectively; SEQ ID NOs: 1913 and 1917 respectively; SEQ ID NOs: 1921 and 1925 respectively; SEQ ID NOs: 1929 and 1933 respectively; SEQ ID NOs: 2265 and 2269 respectively; SEQ ID NOs: 2281 and 2285 respectively; 2289 and 2293 respectively; 2329 and 2333 respectively; and SEQ ID NOs: 2345 and 2349, respectively.

15. The heterodimeric multispecific antibody of claim 1, wherein the first immunoglobulin or the third immunoglobulin
binds to a cell surface antigen selected from the group consisting of a2b b3 (Glycoprotein IIb/IIIa), a4, a4b7, a4b7+aEb7, a5, Activin receptor type-2B, ALK1, Alpha-synuclein, amyloid beta, APP, AXL, Blood Group A, CAIX, CCL-2, CD105 (endoglin), CD115 (CSF1R), CD116a (CSF2Ra), CD123, CD152 (CTLA4), CD184 (CXCR4), CD19, CD192 (CCR2), CD194 (CCR4), CD195 (CCR5), CD20, CD200, CD22, CD221 (IGF1R), CD248, CD25, CD257 (BAFF), CD26, CD262 (DR5), CD276 (B7H3), CD3, CD30 (TNFRSF8), CD319 (SLAMF7), CD33, CD332 (FGFR2), CD350 (FZD10), CD37, CD371 (CLEC12A), CD38, CD4, CD49b (a2), CD51 (a5), CD52, CD56, CD61 (a4b3), CD70, CD73 (NTSE), CD74, CEA, Claudin-18.2, cMET, CRLR, DLL3, DLL4, DNA/histone (H1) complex, EGFR, EpCAM, EGFR-HER3, EGFRvIII, EphA3, ERGT(GalNAc) Tn Antigen, FLT1, FOLR1, frizzled family receptor (FZD), Lewis Y, Lewis X, GCGR, GD2, GD2 α-acetyl, GD3, GM1, GM1 fucosyl, GM2, GPA33, GPNMB, GUCY2C, HER2, HER3, HGFR (cMET), IgHe, IGLF2, Kallikreins, LINGO1, LOXL2, Ly6/PLAUR domain-containing protein 3, MADCAM1, MAG, Mesothelin, MT1-MMP (MMP14), MUC1, Mucin SAC, NaPi2b, NeuGc-GM3, notch, NOTCH2/NOTCH3 receptors, oxLDL, P-selectin, PCSK9, PDGFRA, PDGFRa, phosphatidylserine, polysialic acid, PSMA, PVRL4, RGMA, CD240D Blood group D antigen, root plate-specific spondin 3, serum amyloid P component, STEAP-1, TACSTD2, TGFb, TWEAKR, TYRP1, VEGFR2, VSIR, CD171 (L1CAM), CD19, CD47, pMHC[NY-ESO1], pMHC[MART1], pMHC[MAGEA1], pMHC[Tyrosinase], pMHC[gp100], pMHC[MUC1], pMHC[tax], pMHC[WT-1], pMHC[EBNA-1], pMHC[LMP2], pMHC[hTERT], GPC3, CD80, CD23, and fibronectin extra domain-B, or
bind to two different epitopes on a target cell, optionally wherein the target cell is a cancer cell.

16. (canceled)

17. (canceled)

18. The heterodimeric multispecific antibody of claim 1, wherein the second immunoglobulin or the fourth immunoglobulin
bind to an epitope on a white blood cell, a monocyte, a lymphocyte, a granulocyte, a macrophage, a T cell, a NK cell, a B cell, a NKT cell, an ILC, or neutrophil, or
bind to an antigen selected from the group consisting of Dabigatran, a4, a4b7, a4b7 +aEb7, a5, AXL, BnDOTA, CD11a (LFA-1), CD3, CD4, CD8, CD16, CD19, CD22, CD23, CD25, CD28, CD30 (TNFRSF8), CD33, CD38, CD40, CD40L, CD47, CD49b (a2), CD54 (ICAM-1), CD56, CD74, CD80, CD115 (CSF1R), CD116a (CSF2Ra), CD123, CD134 (OX40), CD137 (41BB), CD152 (CTLA4), CD184 (CXCR4), CD192 (CCR2), CD194 (CCR4), CD195 (CCR5), CD223 (LAG-3), CD252 (OX40L), CD254 (RANKL), CD262 (DR5), CD27, CD200, CD221 (IGF1R), CD248, CD274 (PD-L1), CD275 (ICOS-L), CD278 (ICOS), CD279 (PD-1), CD319 (SLAMF7), CD371 (CLEC12A), MADCAM1, MT1-MMP (MMP14), NKG2A, NRP1,TIGIT, VSIR, KIRDL1/2/3, and KIR2DL2, or
bind to two different epitopes on a white blood cell, a monocyte, a lymphocyte, a granulocyte, a macrophage, a T cell, a NK cell, a B cell, a NKT cell, an ILC, or neutrophil.

19. (canceled)

20. (canceled)

21. The heterodimeric multispecific antibody of claim 1, wherein
the second immunoglobulin binds CD3 and the fourth immunoglobulin binds an immune cell receptor selected from the group consisting of CD4, CD8, CD25, CD28, CTLA4, OX40, ICOS, PD-1, PD-L1, 41BB, CD2, CD69, and CD45, or
the second immunoglobulin binds CD16 and the fourth immunoglobulin binds an immune cell receptor selected from the group consisting of CD56, NKG2D, and KIRDL1/2/3, or
wherein the fourth immunoglobulin binds to an agent selected from the group consisting of a cytokine, a nucleic acid, a hapten, a small molecule, a radionuclide, an immunotoxin, a vitamin, a peptide, a lipid, a carbohydrate, biotin, digoxin, or any conjugated variants thereof, or
wherein the antibody is a monoclonal antibody, a chimeric antibody, or a humanized antibody.

22. (canceled)

23. (canceled)

24. The heterodimeric multispecific antibody of claim 1, wherein the first immunoglobulin and the third immunoglobulin
bind to their respective epitopes with a monovalent affinity or an effective affinity between about 100 nM to about 100 pM, or
bind to cell surface epitopes that are between 60 and 120 angstroms apart, or
bind to their respective epitopes with a monovalent affinity or an effective affinity that is less than 100 pM, or
bind to cell surface epitopes that are up to 180 angstroms apart.

25. (canceled)

26. (canceled)

27. (canceled)

28. The heterodimeric multispecific antibody of claim 1, wherein the first heterodimerization domain and/or the second heterodimerization domain is a CH2-CH3 domain and has an isotype selected from the group consisting of IgG1, IgG2, IgG3, IgG4, IgA1, IgA2, IgM, IgD, and IgE, optionally wherein
the first heterodimerization domain and/or the second heterodimerization domain is an IgG1 constant region comprising one or more amino acid substitutions selected from the group consisting of N297A and K322A, or
the first heterodimerization domain is a CH2-CH3 domain comprising a K409R mutation and the second heterodimerization domain is a CH2-CH3 domain comprising a F405L mutation.

29. (canceled)

30. (canceled)

31. (canceled)

32. A recombinant nucleic acid sequence encoding the heterodimeric multispecific antibody of claim 1.

33. A host cell or vector comprising the recombinant nucleic acid sequence of claim 32.

34. A composition comprising the heterodimeric multispecific antibody of claim 1 and a pharmaceutically-acceptable carrier, wherein the antibody is optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof.

35. A method for treating cancer in a subject in need thereof, comprising administering to the subject an effective amount of the heterodimeric multispecific antibody of claim 1, optionally wherein
the cancer is selected from the group consisting of lung cancer, colorectal cancer, skin cancer, breast cancer, ovarian cancer, leukemia, pancreatic cancer, and gastric cancer, or
the heterodimeric multispecific antibody is administered to the subject separately, sequentially or simultaneously with an additional therapeutic agent.

36. (canceled)

37. (canceled)

38. A kit comprising the heterodimeric multispecific antibody of claim 1, and instructions for use.
